(19) United States
(12) Patent Application Publication
(10) Pub. No.: US 2026/0168000 A1
(43) Pub. Date: Jun. 18, 2026

(54) GENETICALLY MODIFIED BENZYLISOQUINOLINE ALKALOID-PRODUCING HOST CELLS WITH MODIFIED EFFLUX TRANSPORTER GENE EXPRESSION

(71) Applicant: RIVER STONE BIOTECH APS, Copenhagen (DK)

(72) Inventors: Esben Halkjaer HANSEN, Copenhagen (DK); Laura Tatjer RECORDA, Copenhagen (DK); Pernille Lund LYNEBORG, Copenhagen (DK); Giedre BACINSKAJA, Copenhagen (DK); Jens HOUGHTON-LARSEN, Copenhagen (DK)

(21) Appl. No.: 19/127,269

(22) PCT Filed: Nov. 7, 2023
(86) PCT No.: PCT/EP2023/081034

§ 371 Date: May 5, 2025

(30) Foreign Application Priority Data
Nov. 8, 2022 (EP) 22206111.1

(51) Int. Cl. C12P 17/18; C07K 14/395; C12N 1/18; C12N 9/02; C12N 9/42; C12N 15/52; C12P 19/60; C12R 1/865

(52) U.S. Cl. CPC C12P 17/188; C07K 14/395; C12N 1/18; C12N 9/0042; C12N 9/0071; C12N 9/2445; C12N 15/52; C12P 19/60; C12R 2001/865; C12Y 106/02004; C12Y 114/14001; C12Y 302/01021

(57) ABSTRACT

The invention relates to genetically modified hosts cell comprising a recombinant pathway having enhanced production of one or more benzylisoquinoline alkaloids or glycosylated benzylisoquinoline alkaloid, wherein the host cell has been further modified so as to have an increased ability to export end products of the recombinant pathway (such as nororipavine or gly-nororipavine).

### FIELD OF THE INVENTION

The present disclosure relates to methods of producing benzylisoquinoline alkaloids (one or more BIA, BIA-glycoside, oripavine, glucosylated oripavine, gly-oripavine, thebaine, northebaine, nororipavine, gly-nororipavine, glucosylated nororipavine, nor-opioids or glycosylated noropioids), by the use of host cells genetically modified to (i) express one or more genes in an operative metabolic pathway producing the benzylisoquinoline alkaloids and (ii) modified expression of BIA-relevant efflux transporters.

### BACKGROUND OF THE INVENTION

Effective production of pharmaceutical opioids by biotransformation, such as wholly or partly by fermentation of genetically engineered strains and/or by bioconversion, requires complex engineering and optimization of metabolic pathways producing the opioids or their precursors and optionally further chemical modifications. Some pharmaceutical opioids with desirable pharmacological properties, such as buprenorphine, naltrexone, naloxone and nalbuphine require demethylation of benzylisoquinoline alkaloids (BIAs) such as thebaine and/or oripavine and an N-alkylation of the demethylated benzylisoquinoline alkaloid, such as for the production of buprenorphine from nororipavine. Such production of BIAs using genetically modified host cell cultures expressing one or more genes in an operative metabolic pathway producing the benzylisoquinoline alkaloids has previously been disclosed by this research group in WO2021/069714, the contents of which are incorporated herein in their entirety.

However, for even greater improvements in the efficiently of producing pharmaceutical opioids there is a need for improving and optimising both pathways in genetically modified microbial strains producing BIA products (such as noropioids and glycosylated-noropioids) as well as enhanced efflux of the desirable BIA products out of the modified host cells, so as to enhance titers from cultures of recombinant microbial host cells, aid purification of the BIA products therefrom, and/or permit production of the BIA products from batch, fed-batch, semi-continuous, or continuous fermentation of the recombinant microbial host cells. Improved efflux of BIAs and/or glycosylated BIAs (such as oripavine, gly-oripavine, nororipavine and gly-nororipavine) have also surprisingly been observed to increase culture biomass and therefore increase production efficiency further from recombinant microbial host cells.

Earlier opiod strain development work was focused on or suggested the deletion of multidrug or ABC efflux transporters to prevent efflux of important intermediates in the pathway, or induction of these efflux transporters' expression during the stationary phase once production of the opioid is finished (US 2018/334695), as well as identifying these transporters from BIA-producing plants such as poppies. WO 2019/243624 suggests the downregulation or deletion of transporters that excrete intermediates in the BIA pathways, notably molecules that are prior to formation of a BIA molecule. Similarly Narcross et al. 2016 suggested that knocking out transporters could be a means to controlling the ratio of BIA end products. In the literature that does suggest overexpression of transporters for production of BIAs, as opposed to downregulation/knock out, the types of transporters cited are generally of the purine permease family of transporters (PUP). These are used in processes with demethylases for uptake of substrates such as thebaine, oripavine, or northebaine to allow for more efficient conversion to the demethylated endproducts (WO 2021/069714 A1, WO2020/078837, WO 2018/229306 for example). Because permeases can be involved in efflux and uptake, efflux can not be ruled out; however, PUPs were selected in this body of work that were specific to the uptake of substrate molecules and not the demethylated endproducts.

### SUMMARY OF THE INVENTION

Beyond the background art of several pathway enzymes capable of contributing to more efficient production of high specificity and purity, BIAs and/or BIA derivatives, in recombinant host cells, the work presented herein discloses benefits of modifying within such host cells the efflux (outward transportation) of BIAs and/or BIA derivatives (one or more BIA, BIA-glycoside, oripavine, glucosylated oripavine, gly-oripavine, thebaine, northebaine, nororipavine, gly-nororipavine, glucosylated nororipavine, nor-opioids or glycosylated noropioids by selecting a specific family and subfamily of efflux transporters with specificity for the products of interest compared to intermediates and substrates. Modification of the recombinant host cell's transport system may be done by upregulation of endogenous host cell transporters shown herein to efflux desirable BIAs and/or BIA derivatives. Alternatively, or additionally, modification of the recombinant host cell's transport system may be done by functional addition (and optionally high expression) of heterologous transporters shown herein to efflux desirable BIAs and/or BIA derivatives. In some aspects, the BIA-transporters are able to efflux from the host cell glycosylated derivatives of the desirable BIAs (such as but not limited to glycosylated nororipavine). Glycosylation of nororipavine not only produces a hitherto unknown opioid glycoside, which possesses interesting properties and improves production of nor compounds by the cell, but in vivo expression of efflux transporters also offers a range of hitherto unknown advantages in processes of producing glycosylated nororipavine in genetically modified cell factories, such as yeast, including but not limited to: excretion and separation of nororipavine glycosides from the cells which prevents (i) intracellular nororipavine degradation; (ii) acidification of the yeast cytosol and the stress associated with repeated cycles of excretion and proton driven uptake of nororipavine; (iii) inhibition of oripavine uptake by unwanted competitive uptake of extracellular nororipavine; (iv) product inhibition of the oripavine demethylase enzyme by presence of high concentrations of nororipavine; and (v) increase in propagation and biomass production of genetically modified cell factories glycosylating nororipavine.

A membrane transport protein (or simply transporter) is a membrane protein involved in the movement of ions, small molecules, or macromolecules, such as peptides, across a biological membrane. As discussed in the article of Brohée et al. (“YTPdb: A wiki database of yeast membrane transporters”; Biochimica et Biophysica Acta 1798 (2010) 1908-1912)—among the 5690 protein-encoding genes of the yeast Saccharomyces Cerevisiae—almost 300 code for established or predicted membrane transport (transporter) proteins. The Saccharomyces Genome Database (SGD) (https://www.yeastgenome.org) provides a description of all protein-encoding genes of the yeast Saccharomyces Cerevisiae—for instance is said in relation to the membrane transporter with standard name “QDR2” that is a transporter that “has broad substrate specificity and can transport many mono- and divalent cations” (https://www.yeastgenome.org/locus/S000001383).

As disclosed herein, the present inventors tested overexpression of a number of endogenous and heterologous membrane transporter related genes which could potentially have an influence on the yield of in vivo production of nororipavine and glycosylated noripavine as well as thebaine, oripavine and glycosylated oripavine, which are precursors for synthesis routes to known active pharmaceutical ingredients (“APIs”) such as opioid compounds.

Furthermore, as disclosed herein, the present inventors also tested overexpression of endogenous yeast transporters (e.g. YOR1 and PDR5) and expression of a number of heterologous (non-native) membrane transporter genes which could potentially have an influence on the yield of in vivo bioconversion of oripavine or thebaine to relevant downstream opioid biosynthesis compounds. As discussed in the working examples herein, the inventors were also able to identify a number of heterologous membrane transporter genes that were observed to have a positive effect on the yield of in vivo bioconversion and/or production of BIAs and BIA derivatives, such as oripavine conversion to nororipavine and glucosylated nororipavine. Without being limited to theory, it is contemplated that the improved positive yield effect demonstrated herein related to the expression of certain heterologous membrane transporter genes could be related to a purported ability of those heterologous transporters to efflux BIAs and/or BIA derivatives, which not only permits easier purification and downstream processing, but may also facilitate more efficient enzyme conversions by removing the products formed and thereby create better “sink” conditions driving the chemical reactions leading to the desired BIAs and/or BIA derivatives.

Accordingly, the present invention provides in a first aspect a genetically modified (recombinant) microbial host cell capable of producing one or more benzylisoquinoline alkaloids (BIAs) (such as BIA-glycoside, oripavine or oripavine glycoside or glucosylated oripavine, thebaine, northebaine, nororipavine or gly-nororipavine or glucosylated nororipavine) wherein the host cell comprises a recombinant polynucleotide comprising a promoter operably linked to an ABC transporter capable of effluxing one or more BIAs or glycosylated BIAs. In some aspects, the recombinant microbial host cell of the current invention comprises an ABC transporter which is an ABCC/multi-drug resistance associated protein (MRP) ABC transporters or an ABCG/pleiotropic drug resistance (PDR) ABC transporters. In some aspects, BIA-relevant (such as BIA-glycoside, oripavine or gly-oripavine or glucosylated oripavine, thebaine, northebaine, nororipavine or gly-nororipavine or glucosylated nororipavine) efflux transporters are ABC transporters. In some aspects, the BIA-relevant (BIA-glycoside, oripavine, thebaine, northebaine, nororipavine or gly-nororipavine or glucosylated nororipavine) efflux transporters are members of the ABCG/pleiotropic drug resistance (PDR) subfamily of ABC transporters, or members of the ABCC/multi-drug resistance associated protein (MRP) subfamily of ABC transporters.

In some aspects, the current invention provides genetically modified (recombinant) microbial host cell capable of producing one or more benzylisoquinoline alkaloids and comprising one or more ABC transporters (such as ABCC or ABCG transporters) capable of effluxing the BIA, BIA-glycoside, oripavine, thebaine, northebaine, nororipavine, gly-nororipavine, glucosylated nororipavine nor-opioids or glycosylated noropioids, may comprise the following enzymes involved in the production of the BIA:
(1) one or more heterologous CYP demethylases capable of converting thebaine into northebaine, thebaine into oripavine, northebaine into nororipavine and/or oripavine into nororipavine, and one or more demethylase cytochrome P450 reductase (demethylase-CPR), and/or
(2) heterologous sequences encoding:
   (a) a tyrosine hydroxylase (TH) converting L-tyrosine into L-dopa, and
   (b) optionally, a TH-CPR capable of reducing the TH of (a); and
   (c) a L-dopa decarboxylase (DODC) converting L-dopa into dopamine, or a tyrosine decarboxylase (TYDC) converting L-dopa into dopamine; and
   (d) a monoamine oxidase converting dopamine into 3,4-DHPAA, or a N-methyl-coclaurine hydroxylase (NMCH) converting (S)-Coclaurine into (S)-3′-hydroxycoclaurine and/or (S)—N-Methylcoclaurine into (S)-3′-Hydroxy-N-Methylcoclaurine; and
   (e) a norcoclaurine synthase (NCS) converting Dopamine and 4-HPAA into (S)-norcoclaurine and/or 3,4-DHPAA and dopamine to norlaudanosoline; and
   (f) a 6-O-methyltransferase (6-OMT) converting (S)-norcoclaurine into (S)-Coclaurine and/or norlaudanosoline into (S)-3′-Hydroxy-coclaurine; and
   (g) a coclaurine-N-methyltransferase (CNMT) converting (S)-Coclaurine into (S)—N-Methylcoclaurine and/or (S)-3′-hydroxycoclaurine into (S)-3′-hydroxy-N-methyl-coclaurine; and
   (h) a 3′-hydroxy-N-methyl-(S)-coclaurine 4′-O-methyltransferase (4′-OMT) converting (S)-3′-Hydroxy-N-Methylcoclaurine into (S)-reticuline; and
   (i) a 1,2-dehydroreticuline synthase-1,2-dehydroreticuline reductase (DRS-DRR) converting (S)-reticuline into (R)-reticuline comprised of one or more proteins; and
   (j) a salutaridine synthase (SAS) converting (R)-reticuline into Salutaridine; and
   (k) a salutaridine reductase (SAR) converting Salutaridine to Salutaridinol; and
   (l) a salutaridinol 7-O-acetyltransferase (SAT) converting Salutaridinol into 7-O-acetylsalutaridinol; and
   (m) a thebaine synthase (THS) converting 7-O-acetylsalutaridinol or 7-O-acetylsalutaridinol acetate into thebaine;
(3) and optionally, one or more glycosyl transferases capable of transferring a glycosyl moiety to the BIA (such as oripavine or nororipavine).

In some aspects, the host cell produces BIAs from a precursor molecule such as thebaine or oripavine, and requires one or more heterologous demethylase enzymes and optionally a demethylase-CPR and/or one or more glycosyl transferases capable of transferring a glycosyl moiety to oripavine or nororipavine. In some aspects, the host produces BIAs de novo via a pathway from tyrosine to thebaine (and thence to downstream BIAs) and comprises the enzyme activities: TYRH, DODC, NCS, 6OMT, CNMT, NMCH, 4OMT, DRS-DRR, SAS, SAR, SAT, THS and if the BIA is downstream of thebaine, optionally one or more demethylases converting thebaine into oripavine, thebaine into northebaine, oripavine into nororipavine and/or northebaine into nororipavine, and optionally a demethylase-CPR capable of reducing the demethylase, optionally, one or more glycosyl transferases capable of transferring a glycosyl moiety to oripavine or nororipavine. In other aspects, the pathway from tyrosine to thebaine (and thence to downstream BIAs) comprises the enzyme activities: TYRH, DODC, MAO, NCS, 6OMT, CNMT, 4 OMT, DRS-DRR, sal synthase, sal reductase, sat1, thebaine synthase.

In another aspect, the present invention provides:
1) a pathway having enhanced production of one or more benzylisoquinoline alkaloids (BIAs) or benzylisoquinoline alkaloids derivatives, wherein the cell comprises one or more features selected from:
   a) expression of one or more heterologous genes encoding a demethylase capable of converting thebaine into northebaine, thebaine into oripavine, northebaine into nororipavine and/or oripavine into nororipavine;
   b) expression of one or more heterologous genes encoding a tyrosine hydroxylase (TH) converting L-tyrosine into L-dopa, such as one or more TH having at least 70% identity, such as at least 75% identity, such as at least 80%, such as at least 85%, such as at least 90%, such as at least 95%, such as at least 99%, such as 100% identity to the TH comprised in SEQ ID No. 7, 9, 11, 13, 15, 17, 19, 21, 23, 25, 27, 29, 31, 33, 35, 37, 39, 41, 43, 45, 47, 49, 51, 53, 55, 57, 59, 61, 63 or 65;
   c) reduction or elimination of activity of one or more dehydrogenases native to the host cell, such as one or more dehydrogenases having at least 70% identity, such as at least 75% identity, such as at least 80%, such as at least 85%, such as at least 90%, such as at least 95%, such as at least 99%, such as 100% identity to the dehydrogenase comprised in SEQ ID NO: 663, 665, 667, 669, 671, 673, 675, 677, 679, 681, 683, 685, 687, 689, 691, 693, 695, 697, 699, 701, 703 or 705;
   d) reduction or elimination of activity of one or more reductases native to the host cell, such as one or more reductases having at least 70% identity, such as at least 75% identity, such as at least 80%, such as at least 85%, such as at least 90%, such as at least 95%, such as at least 99%, such as 100% identity to the reductase comprised in SEQ ID NO: 707, 709, 711, 713, 715, 717, 719, 721, 723, 725, 727, 729 or 731;
   e) expression of one or more heterologous genes encoding a norcoclaurine synthase (NCS) converting Dopamine and 4-HPAA into (S)-norcoclaurine, such as one or more NCS having at least 70% identity, such as at least 75% identity, such as at least 80%, such as at least 85%, such as at least 90%, such as at least 95%, such as at least 99%, such as 100% identity to the NCS comprised in SEQ ID NO: 73 OR 76;
   f) expression of one or more heterologous genes encoding:
   i) a fused 1,2-dehydroreticuline synthase-1,2-dehydroreticuline reductase (DRS-DRR) converting (S)-Reticuline into (R)-reticuline, wherein
      ia) the DRS-DDR has at least 70% identity, such as at least 75% identity, such as at least 80%, such as at least 85%, such as at least 90%, such as at least 95%, such as at least 99%, such as 100% identity to the DRS-DRR comprised in SEQ ID NO: 92, 94, 96; or
      ib) the DRS moiety has at least 70% identity, such as at least 75% identity, such as at least 80%, such as at least 85%, such as at least 90%, such as at least 95%, such as at least 99%, such as 100% identity to the DRS comprised in SEQ ID NO: 98, 100, 102, 104 or 106; and the DRR moiety has at least 70% identity, such as at least 75% identity, such as at least 80%, such as at least 85%, such as at least 90%, such as at least 95%, such as at least 99%, such as 100% identity to the DRR comprised in SEQ ID NO: 108 or 110; or
   ii) a DRS having at least at least 70% identity, such as 75% identity, such as at least 80%, such as at least 85%, such as at least 90%, such as at least 95%, such as at least 99%, such as 100% identity to the DRS comprised in SEQ ID NO: 98, 100, 102, 104 or 106; and a DRR having at least 70% identity to the DRR comprised in SEQ ID NO: 108 or 110;
   iii) a fused 1,2-dehydroreticuline synthase-1,2-dehydroreticuline reductase (DRS-DRR) converting (S)-Reticuline into (R)-reticuline, wherein the fused DRS-DRR has at least 70% identity, such as at least 75% identity, such as at least 80%, such as at least 85%, such as at least 90%, such as at least 95%, such as at least 99%, such as 100% identity to the DRS-DRR comprised in SEQ ID NO: 92, 94, 96; and/or
   iv) a 1,2-dehydroreticuline synthase (DRS) and a 1,2-dehydroreticuline reductases (DDR), wherein the DRS has at least 75% identity, such as at least 80%, such as at least 85%, such as at least 90%, such as at least 95%, such as at least 99%, such as 100% identity to the DRS comprised in SEQ ID NO: 98, 100, 102, 104 or 106; and the DDR has at least 70%, such as at least 75% identity, such as at least 80%, such as at least 85%, such as at least 90%, such as at least 95%, such as at least 99%, such as 100% identity to the DRR comprised in SEQ ID NO: 108 or 110;
   g) expression of one or more heterologous genes encoding a thebaine synthase (THS) converting 7-O-acetylsalutaridinol into thebaine, such as one or more THS having at least 70%, such as at least 75% identity, such as at least 80%, such as at least 85%, such as at least 90%, such as at least 95%, such as at least 99%, such as 100% identity to the THS comprised in SEQ ID NO: 126, 127, 128, 129, 131, 133, 134, 136 or 138; and
   h) expression of one or more heterologous genes encoding a transporter protein capable of increasing uptake in the host cell of a reticuline derivative, such as one or more transporter proteins having at least 70%, such as at least 75% identity, such as at least 80%, such as at least 85%, such as at least 90%, such as at least 95%, such as at least 99%, such as 100% identity to the transporter protein comprised in SEQ ID NO: 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 733, 735, 774, 776, 778, 780, 782, 784, 786, 788, 790, 792, 794, 795, 797, 799, 801, 803, 805, 807, 809, 811, 813, 815, 817, 819, 821, 823 or 825,
2) modified expression of one or more BIA-relevant (such as nororipavine and/or gly-nororipavine) efflux transporters, wherein the cell comprises one or more features selected from:
   a) overexpression of one or more of the recombinant host cell's endogenous ABC transporters capable of effluxing desirable BIAs and/or BIA derivatives (one or more BIA, BIA-glycoside, oripavine, glucosylated oripavine, gly-oripavine, thebaine, northebaine, nororipavine, gly-nororipavine, glucosylated nororipavine, nor-opioids or glycosylated noropioids), including but not limited to one or more ABC transporter comprising Walker A sequences G (S/A/L/V/M/P) IG (T/S) GK and GRTGAGK and the linker sequences LSGGQ and NFSLGE, and having at least 45%, such as at least 50%, such as at least 55%, having at least 60%, such as at least 75%, such as at least 80%, such as at least 85%, such as at least 90%, such as at least 95%, such as at least 99%, or 100% identity to SEQ ID No. 872, and/or
   b) the functional addition into the recombinant host cell of one or more exogenous ABC transporter capable of effluxing desirable BIAs and/or BIA derivatives (one or more BIA, BIA-glycoside, oripavine, glucosylated oripavine, gly-oripavine, thebaine, northebaine, nororipavine, gly-nororipavine, glucosylated nororipavine, nor-opioids or glycosylated noropioids), such as one or more exogenous ABC transporter:
      i. that is an ABCC/multi-drug resistance associated protein (MRP) ABC transporters, such as an ABC transporter comprising Walker A sequences G(X)(I/V)G(S/T)GK where X is a residue selected from P, L, S, A, V or M and GRTGAGK, two linker sequences comprising LSGGQ and NFSLGE, and Walker B sequences (I/V/T)(I/Y/V)L(M/F/L)D and I(I/L)(I/V)(L/M)D, such as an ABC transporter having at least 45%, such as at least 50%, such as at least 55%, having at least 60%, such as at least 75%, such as at least 80%, such as at least 85%, such as at least 90%, such as at least 95%, such as at least 99%, or 100% identity to SEQ ID No. 910, 912, 914, 918, 920, 922, 924, 926, 928, 930, 932, 934, 936, 938, 940, 942, 956, 960, 962, 964, 966, 970, 1032, 1034, or 1040, or encoded by a nucleic acid sequence having at least 45%, such as at least 60%, such as at least 70%, such as at least 80%, such as at least 85%, such as at least 90%, such as at least 95%, such as at least 99%, such as 100% identity to SEQ ID No. 871, 909, 911, 913, 917, 919, 921, 923, 925, 927, 929, 931, 933, 935, 937, 939, 941, 955, 959, 961, 963, 965, 969, 1031, 1033, or 1039, or genomic DNA thereof, or,
      ii. that is an ABCG/pleiotropic drug resistance (PDR) ABC transporters, such as an ABC transporter comprising Walker A sequences GRPGSGC(S/T) and G(A/S)SGAGKT, S sequences VSGGERKRVSIA and LNVEQRKRLTIG, and Walker B sequences (F/L)QCWD and LL(V/L)F(L/F)D, such as an ABC transporter having at least 45%, such as at least 50%, such as at least 55%, having at least 60%, such as at least 75%, such as at least 80%, such as at least 85%, such as at least 90%, such as at least 95%, such as at least 99%, or 100% identity to SEQ ID No. 916, 976, 980, 986, 988, 990, 994, 996, 1010, 1012, 1018, 1020, 1022, 1026, 1028, 1030 or 1038, or encoded by a nucleic acid sequence having at least 45%, such as at least 60%, such as at least 70%, such as at least 80%, such as at least 85%, such as at least 90%, such as at least 95%, such as at least 99%, such as 100% identity to SEQ ID No. 915, 975, 979, 985, 987, 989, 993, 995, 1009, 1011, 1017, 1019, 1021, 1025, 1027, 1029 or 1037, or genomic DNA thereof.

In a further aspect the invention provides a recombinant host cell comprising a recombinant polynucleotide sequence encoding a heterologous efflux transporter protein of the invention operably linked to one or more control sequences.

In a further aspect the invention provides a cell culture, comprising the recombinant host cell of the invention and a growth medium.

In a further aspect the invention provides a method for producing a BIA and/or BIA derivative (one or more BIA, BIA-glycoside, oripavine, glucosylated oripavine, gly-oripavine, thebaine, northebaine, nororipavine, gly-nororipavine, glucosylated nororipavine, nor-opioids or glycosylated noropioids), comprising:
a) culturing the cell culture of the invention at conditions allowing the recombinant host cell to produce the BIA and/or BIA derivative; and
b) optionally recovering and/or isolating the BIA and/or BIA derivative.

In many aspects, the ABC transporters of the current invention have been selected on the basis of increased specificity for the BIAs and/or BIA derivatives produced by the recombinant host cell (one or more BIA, BIA-glycoside, oripavine, glucosylated oripavine, gly-oripavine, thebaine, northebaine, nororipavine, gly-nororipavine, glucosylated nororipavine, nor-opioids or glycosylated noropioids) compared to one or more intermediate molecules in or substrates fed into the BIA-producing biosynthetic pathway engineered into the recombinant host cell.

In a further aspect the invention provides a fermentation composition comprising the cell culture of the invention and the BIA and/or BIA derivative (one or more BIA, BIA-glycoside, oripavine, glucosylated oripavine, gly-oripavine, thebaine, northebaine, nororipavine, gly-nororipavine, glucosylated nororipavine, nor-opioids or glycosylated noropioids) comprised therein.

In a further aspect the invention provides a composition comprising the fermentation composition of the invention and one or more carriers, agents, additives and/or excipients.

In a further aspect the invention provides a pharmaceutical composition comprising the fermentation composition of the invention and one or more pharmaceutical grade excipient, additives and/or adjuvants.

In a further aspect the invention provides a method for preparing the pharmaceutical composition of the invention comprising mixing the fermentation composition of the invention with one or more pharmaceutical grade excipient, additives and/or adjuvants.

In a further aspect the invention provides a method for preventing, treating and/or relieving a disease comprising administering a therapeutically effective amount of the pharmaceutical composition of the invention to a mammal.

### DESCRIPTION OF DRAWINGS AND FIGURES

FIG. 1 shows the pathway for making the benzylisoquinoline alkaloid precursor tyrosine via the Shikimate pathway and additional steps for producing(s)-norcoclaurine.

FIG. 2 depicts a range of benzylisoquinoline alkaloid compounds having pharmaceutical properties which are derivatives of (S)-norcoclaurine.

FIG. 3 shows a schematic representation of the biosynthetic pathway from glucose to thebaine in genetically modified S. cerevisiae strains. Enzymes from NCS to SAT/THS as well as Tyrosine hydroxylase (TH) and DOPA decarboxylase (DODC) are enzymes expressed from heterologous genes.

FIG. 4 shows Glucosylated nororipavine measured outside and inside the cells normalized to a negative control cell (strain with empty plasmid RPB15).

FIG. 5 shows improvement in fermentation titers of total nororipavine (both glycosylated and unglycosylated) when strains expressing heterologous efflux transporters are used.

FIG. 6. shows nororipavine export by various YOR1 homologs.

FIGS. 7a and 7b show microtiter-based screening of nororipavine and nororipavine-glu producing strains expressing PDR5 homologs.

FIG. 8a. shows 96-deepwell plate screening of oripavine producing strains demonstrating the impact of transporter expression on total bioconversion. Triplicate values of oripavine production by expression of different exporters were normalized to the average oripavine production number obtained by sOD1133 transformed with the empty plasmid, RPB15. The average and Standard deviations of the normalized triplicates are shown.

FIG. 8b. shows transporters exhibiting a reduction in thebaine to oripavine conversion.

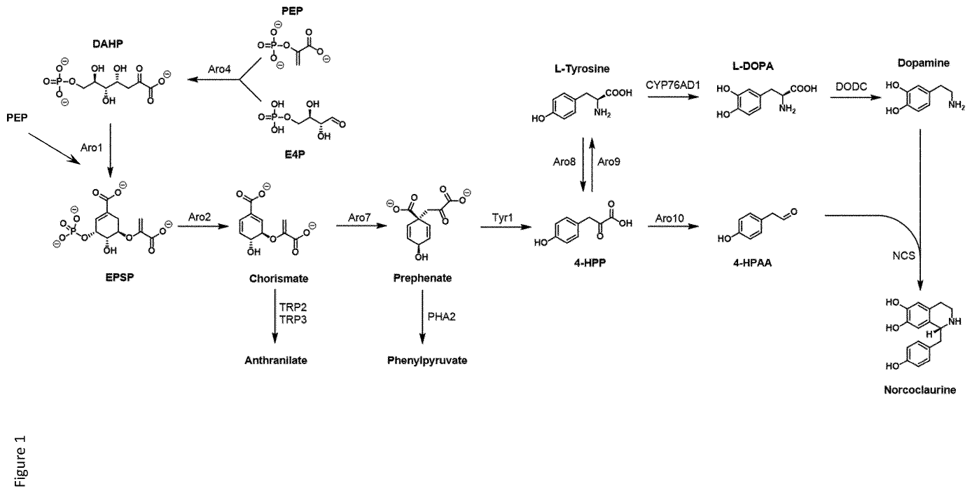

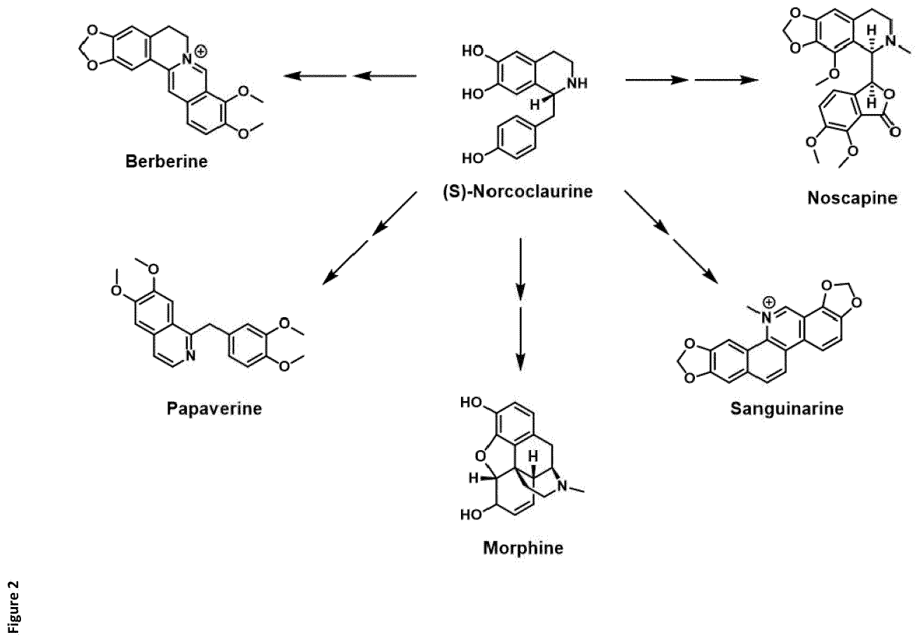

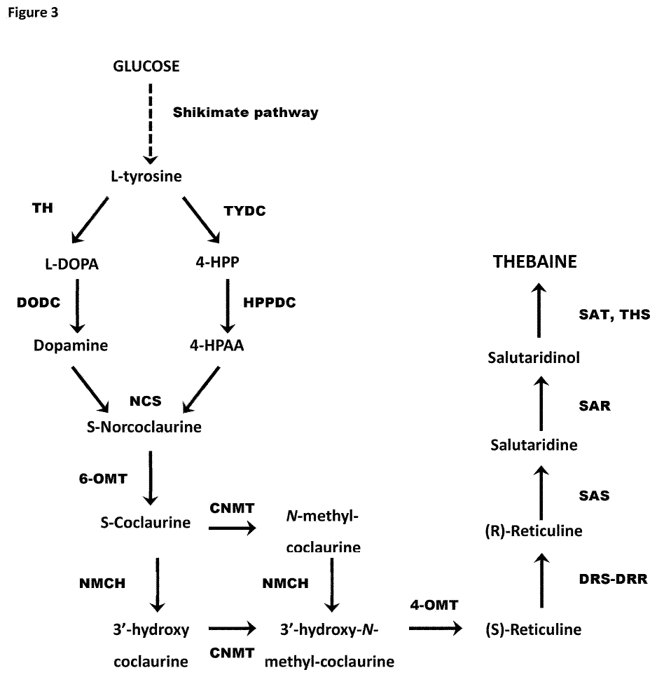

### INCORPORATION BY REFERENCE

All publications, patents, and patent applications referred to herein (including, but not limited to PCT/EP2022/062130, WO2021/069714, WO2018/229306, WO2018/075670, WO2019/243624, WO2018/029282, WO2019/157383, Pyne et al, BioRxiv preprint 2019, WO2018/229305, WO2014/143744, WO2019/165551, US2015267233, WO2015/081437, WO2016/183023, WO2015/173590, WO2018/000089, WO2019/028390, WO2019/165551, WO2018/005553, WO2014/143744, WO2019/165551, WO2020/078837, US2019100781, WO2019/165551, Brohée et al. 2010, Dias et al. 2010, WO2018211331, WO 2021/144362, R J Carroll et al. 2009, Galanie S, et al. 2015, Fossati E et al. 2015, Tomas Hudlicky. 2015, Amitava Dasgupta, 2020) are incorporated by reference to the same extent as if each individual publication, patent, or patent application was specifically and individually indicated to be incorporated by reference. In the event of a conflict between a term herein and a term in an incorporated reference, the term herein prevails and controls.

### DETAILED DESCRIPTION OF THE INVENTION

### Definitions

Any EC numbers used herein refers to Enzyme Nomenclature 1992 from NC-IUBMB, Academic Press, San Diego, California, including 30 supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The nomenclature is regularly supplemented and updated; see e.g. http://enzyme.expasy.org/.

Drug transport proteins can be categorized into two major classes that include solute carriers (SLC) and ATP-Binding Cassette (ABC) transporters. From the human genome around 380 unique SLC sequences have been obtained which can be further divided into 48 sub families. The xenobiotics transport activities for around 19 of these gene families were described. These transporters include organic anion transporting polypeptide (OATP), oligopeptide transporter, organic anion/cation/zwitter ion transporter and organic cation transporter (OCT).

Within the 49 different ABC transporter genes so far identified, seven sub families have so far been categorised. In particular, transporters belonging to the ABCB, ABCC and ABCG sub families have specificities for various drugs. SLC and ABC transporters are involved in the transport of a wide range of substrates and have a wide distribution in the body. Based on the direction of translocation across the cell membrane, the transporter may be categorized as an influx transporter (for uptake into the cell) or an efflux transporter (for excretion out of the cell). ABC transporters are efflux transporters that utilize energy derived from ATP hydrolysis to mediate the active export of molecules from the intracellular to the extracellular mileu, often against a concentration gradient. In contrast, the cellular uptake (influx) of substrates is facilitated by the majority of the SLC family members. However, depending on the concentration gradients of substrate and coupled ion across the membrane, some of the SLC transporters exhibit efflux properties.

It should be noted that especially in recombinant host cells, the intracellular and extracellular concentrations of various pathway precursors, intermediates and final products may well not be similar to those of an unmodified host cell. Additionally, the concentration gradient of each of these precursors, intermediates and final products between the intracellular and extracellular environments will depend on the efficiency of the recombinant pathway producing the BIA or BIA-derivative, and so the beneficial effects disclosed for the first time herein of modifying selected transporter capabilities of host cells may not have been apparent in earlier investigations.

The term “PEP” as used herein refers to phosphoenol pyruvate.

The term “E4P” as used herein refers to erythrose-4-phosphate

The term “Aro4” as used herein refers to DAHP synthase catalyzing the reaction of PEP and E4P into DAHP.

The term “DAHP” as used herein refers to 3-deoxy-D-arabino-2-heptulosonic acid 7-phosphate.

The term “Aro1” as used herein refers to EPSP synthase catalyzing conversion of DAHP into EPSP.

The term “EPSP” as used herein refers to 5-enolpyruvylshikimate-3-phosphate. The term “Aro2” as used herein refers to chorismate synthase catalyzing conversion of EPSP into chorismate.

The term “Tyr1” as used herein refers to prephenate dehydrogenase catalyzing conversion of prephenate into 4-HPP

The term “4-HPP” as used herein refers to 4-hydroxyphenylpyruvate

The term “Aro8” and “Aro9” as used herein refers to aromatic aminotransferase reversibly catalyzing conversion of 4-HPP into L-tyrosine

The term “ARO10” or HPPDC as used herein refers to hydroxyphenylpyruvate decarboxylase catalyzing 4-HPP into 4-HPAA.

The term “4-HPAA” as used herein refers to 4-Hydroxyphenylacetaldehyde.

The term “TH” as used herein refers to a cytochrome P450 enzyme having tyrosine hydroxylase activity and converting L-tyrosine into L-DOPA.

The term “demethylase” as used herein refers to any suitable P450 enzyme, capable of demethylating thebaine into northebaine, thebaine into oripavine, northebaine into nororipavine and/or oripavine into nororipavine. Such a demthylase may have N-demethylation and/or O-demethyation activity. The use of demethylases herein avoids the requirement of expensive chemical demethylations and harsh conditions required for chemical-based conversion processes undesirable in the production of active pharmaceutical ingredients and their intermediates, such as BIAs. For example, the production of nororipavine requires two demethylations if generated from thebaine, and one demethylation if generated from oripavine. The substrates thebaine and/or oripavine may be provided by direct feeding to the recombinant microbial host cells (such as recombinant yeast cells) or can be generated in vivo using a recombinant pathway using glucose, tyrosine, or any intermediate between as the starting substrate. In preferred embodiments, the one or more demethylases have specificity towards producing nor-compounds and produces less by-products.

The terms “glycosylated” and “glucosylated” herein refer to the addition of a glycosyl (carbohydrate) group from a glycosyl donor. A non-limiting example of a glucose donor is UDP-glucose. A non-limiting example of a glycosyl donor protein is SEQ ID No. 899, encoded by SEQ ID No. 900. No Other carbohydrates are also suitable, for example N-acetyl glucosamine, wherein the sugar donor is Uridine diphosphate N-acetylglucosamine. Glycosyl transferases are able to glycosylate opioids and BIAs to produce opioid glycosides and BIA glycosides respectively. In one embodiment, a UDP-glucose glycosyltransferase (referred to herein as a “UGT”) is utilized that improves the recombinant cell's efficiency for converting oripavine to nororipavine and nororipavine glucoside. Suitable UGTs may display aglycone O-UGT activity and/or aglycone O-glucosyltransferase activity. Surprisingly, it has been found that glycosylation with a UGT significantly improves biomass and productivity of nororipavine producing strains (data presented by the current research group in PCT/EP2022/062130, the entire contents of which are hereby incorporated into the disclosure of the current invention). Embodiments of the current invention, may comprise one or more glycosyl transferases (UGT) selected from an amino acid sequence having at least 60%, such as at least 70%, such at least 75%, such as at least 80%, such as at least 90%, such as at least 95%, such as at least 99%, such as 100% identity to a UGT comprised in any one of SEQ ID NO: 878, 880, 882, 884, 886, 888, 890, 892, 894, 896, or 898; or encoded by a nucleic acid sequence having at least 60%, such as at least 70%, such at least 75%, such as at least 80%, such as at least 90%, such as at least 95%, such as at least 99%, such as 100% identity to SEQ ID No. 879, 881, 877, 883, 885, 887, 889, 891, 893, 895 or 897, or genomic DNA thereof.

The term “DRS” as used herein refers to 1,2-dehydroreticuline synthase, a cytochrome P450 enzyme which catalyze conversion of (S)-Reticuline into 1,2-dehydroreticuline.

The term “DRR” as used herein refers to 1,2-dehydroreticuline reductase which catalyzes conversion of 1,2-dehydroreticuline to (R)-Reticuline.

The term “DRS-DRR” as used herein refers to 1,2-dehydroreticuline synthase-1,2-dehydroreticuline reductase fused complex catalyzing conversion of (S)-Reticuline into (R)-reticuline. This complex may also be referred to as STORR or REPI. DRS-DRR or DRS together with DRR are also categorised as epimerases or isomerases.

The term “CPR” as used herein refers to a cytochrome P450 reductase catalyzing the electron transfer (from NADPH) to a cytochrome P450 enzyme of the pathway, typically in the endoplasmic reticulum of a eukaryotic cell. For distinction and as disclosed herein CPR's are divided into demethylase-CPR used for CPR's capable of reducing demethylases; DRS-CPR used for CPR's capable of reducing DRS and TH-CPR used for CPR's capable of reducing TH. Demethylase-CPR, DRS-CPR and TH-CPR may be identical or different, depending on the P450 to be reduced.

The term “Cytochrome P450 enzyme” or “P450 enzymes” or “P450” as used herein interchangeably refers to a family of monooxygenases enzymes containing heme as a cofactor. P450s are also known as “CYPs”. For distinction and as disclosed herein P450 enzymes are divided into demethylase P450s; DRS P450s, and TH P450s.

The term “DODC” and TYDC” as used herein refers to L-dopa decarboxylase and tyrosine decarboxylase respectively capable of catalyzing conversion of L-DOPA into dopamine and tyrosine into 4-HPP.

The term “MAO” as used herein refers to monoamine oxidase capable of catalyzing the conversion of dopamine to 3,4 DHPAA

The term “DHPAA” as used herein refers to 3,4-dihydroxyphenylacetaldehyde.

The term “NCS” as used herein refers to Norcoclaurine synthase capable of catalyzing conversion of dopamine and 4-HPAA into Norcoclaurine.

The term “6-OMT” as used herein refers to 6-O-methyltransferase capable of catalyzing conversion of (S)-norcoclaurine to (S)-Coclaurine

The term “CNMT” as used herein refers to Coclaurine-N-methyltransferase capable of catalyzing conversion of (S)-Coclaurine to (S)—N-Methylcoclaurine and/or (S)-3′-hydroxycoclaurine to (S)-3′-hydroxy-N-methyl-coclaurine.

The term “NMCH” as used herein refers to N-methylcoclaurine 3′-monooxygenase capable of catalyzing conversion of (S)-Coclaurine to (S)-3′-hydroxycoclaurine and/or (S)—N-Methylcoclaurine to (S)-3′-Hydroxy-N-Methylcoclaurine

The term “4′-OMT” as used herein refers to 3′-hydroxy-N-methyl-(S)-coclaurine 4′-O-methyltransferase capable of catalyzing conversion of (S)-3′-Hydroxy-N-Methylcoclaurine to (S)-reticuline.

The term “SAS” as used herein refers to salutaridine synthase capable of catalyzing conversion of (R)-reticuline to Salutaridine.

The term “SAR” as used herein refers to salutaridine reductase capable of catalyzing conversion of Salutaridine to Salutaridinol.

The term “SAT” as used herein refers to salutaridinol 7-O-acetyltransferase capable of catalyzing conversion of Salutaridinol to 7-O-acetylsalutaridinol.

The term “THS” as used herein refers to thebaine synthase capable of catalyzing conversion of 7-O-acetylsalutaridinol into thebaine.

The term “BIA” or “benzylisoquinoline alkaloid” as used herein refers to a compound of the general formula A:

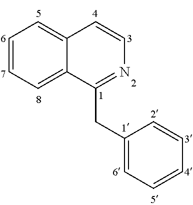

which is the structural backbone of many alkaloids with a wide variety of structures, or to alkaloid products deriving from formula A of the general formula B also known as morphinans:

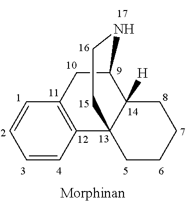

BIAs of relevance to some aspects of the current invention include one or more benzylisoquinoline alkaloid (such as any BIA and/or BIA derivative, BIA-glycoside, oripavine, glucosylated oripavine, gly-oripavine, thebaine, northebaine, nororipavine, gly-nororipavine, glucosylated nororipavine, nor-opioids or glycosylated noropioids).

The term “pathway” or “metabolic pathway” as used herein is intended to mean an enzyme acting in a live cell to convert a chemical substrate into a chemical product. A pathway may include one enzyme or multiple enzymes acting in sequence. A pathway including only one enzyme may also herein be referred to as “bioconversion” in particular relevant for embodiments where the cell of the invention is fed with a precursor or substrate to be converted by the enzyme into a desired benzylisoquinoline alkaloid. Enzymes are characterized by having catalytic activity, which can change the chemical structure of the substrate(s). An enzyme may have more than one substrate and produce more than one product. The enzyme may also depend on cofactors, which can be inorganic chemical compounds or organic compounds (co-factor and/or co-enzymes). The NADPH-dependent cytochrome P450 reductase (CPR) is an electron donor to cytochromes P450 (CYPs). CPR shuttles electrons from NADPH through the Flavin Adenine Dinucleotide (FAD) and Flavin Mononucleotide (FMN) coenzymes into the iron of the prosthetic heme-group of the CYP. The term “operative biosynthetic metabolic pathway” refers to a metabolic pathway that occurs in a live recombinant host, as described herein.

The term “in vivo”, as used herein refers to within a living cell or organism, including, for example animal, a plant or a microorganism.

The term “in vitro”, as used herein refers to outside a living cell or organism, including, without limitation, for example, in a microwell plate, a tube, a flask, a beaker, a tank, a reactor and the like.

The term “in planta”, as used herein refers to within a plant or plant cell.

The term “substrate” or “precursor”, as used herein refers to any compound that can be converted into a different compound. For example, thebaine can be a substrate for P450 and can be converted by demethylation into northebaine. For clarity, substrates and/or precursors include both compounds generated in situ by a enzymatic reaction in a cell or exogenously provided compounds, such as exogenously provided organic molecules which the host cell can metabolize into a desired compound.

The term “endogenous” or “native” as used herein refers to a gene or a polypeptide in a host cell which originates from the same host cell. A cell comprising only endogenous or native genes linked to their native promoters and no recombinant vectors present with extraneous copies will have a “normal” or “typical” phenotype and is referred to by those skilled in the art as “wild type”.

The term “heterologous”, “recombinant”, “genetically modified”, “exogenous” or “non-native” as used herein refers to a polynucleotide, gene or a polypeptide artificially engineered into a host cell that does not normally poses that polynucleotide, gene or polypeptide. As used herein, the term “recombinant polynucleotide sequence” refers to a polynucleotide not found in the wild type cell, and may comprise, for example an endogenous gene linked to a promoter to which it is not operably linked in the wild type cell (i.e. a different native promoter or a heterologous promoter), or a heterologous gene. For example, addition of a heterologous gene permits a recombinant host cell to express the protein encoded by that gene that was not previously part of its wild type genotype; i.e. the heterologous gene originates from a different cell, such as a different strain, variety or species. As used herein, a heterologous polynucleotide may comprise a heterologous gene and/or a different promoter (such as a heterologous promoter, an inducible promoter, a constitutive promoter, a native promoter of higher or weaker strength), etc. Thus, depending on the genetic modification introduced within a host cell, a heterologous polynucleotide may result in the host cell being able to express a heterologous protein, or may result in functional disruption of a native gene, or may result in a native gene being expressed differently to in the wild type cell, such that it may have up-regulated expression (overexpression), down-regulated expression (underexpressed) or inducibly expressed when compared to the wild type host cell.

The term “functional disruption” as used herein refers to manipulation of a gene or any of the machinery participating in the expression the gene, so that said gene no longer expresses a functional version (i.e. not capable of performing the same functions or catalysis) of the polypeptide normally encoded by the unmodified gene in the host cell. Examples of functional disruption include partial or full deletion, frameshift mutation, insertion, removal of part or all of the promoter, or antisense technology. The term “deletion” as used herein refers to manipulation of a gene so that it is no longer expressed in a host cell.

The term “down-regulation”, “down-regulated expression”, and “underexpression” are used interchangeably herein and are understood by one skilled in the art to refer to manipulation of a gene or any of the machinery participating in the expression the gene, so that expression of the gene is reduced as compared to expression without the manipulation.

The term “up-regulation”, “up-regulated expression”, and “overexpression” are used interchangeably herein and are understood by one skilled in the art to refer to manipulation of a gene or any of the machinery participating in the expression the gene, so that expression of the gene is increased as compared to expression without the manipulation.

The term “recombinant host cell” is understood by those skilled in the art to be a cell that has been genetically modified through the deliberate modification of DNA in the cell's genome. As known in the art, recombinant polynucleotide (e.g. DNA) molecules are polynucleotide (e.g. DNA) molecules formed by laboratory methods of genetic recombination (such as molecular cloning) to bring together genetic material from multiple sources, creating sequences that would not otherwise be found in biological organisms. The terms “strain” and “cell” are used interchangeably herein.

As used herein, the terms “influx” or “uptake” refer to movement or pumping of a substrate (such as a BIA) into a cell, and the term “efflux” or “excretion” refers to movement or pumping of a substrate (such as a BIA) out of a cell. In aspects of the current invention, the substrates to be effluxed out of the recombinant microbial host cell is one or more substrate selected from a BIA, BIA-glycoside, oripavine, thebaine, northebaine, nororipavine or glycosylated nororipavine or glucosylated nororipavine.

The terms “ABC transport protein” and “ABC transporter” as used interchangeably herein, refers to a class of ATP-dependent pumps, as recognised by those skilled in the art that comprise an ATP-binding cassette (ABC). The presence of the ATP-binding cassette allows identification of ABC transporters by sequence homology searches to the consensus sequence of the conserved ATP-binding cassette, commonly referred to by those skilled in this art as Walker A sequence (also called the P-loop motif because of its role in phosphate binding) and a downstream more variable Walker B sequence. The Walker A sequence has the consensus sequence G-x(4)-GK-[T/S], where G, K, T and S denote glycine, lysine, threonine and serine residues respectively, and x denotes any amino acid. The Walker B sequence is far more variable, but always comprises a negatively charged residue following a stretch of bulky, hydrophobic amino acids. A somewhat conserved “Linker” sequence, also known as an “S” or “C” sequence, is present in between the Walker A and Walker B motifs, which reside in cytosolic region of the cell.

The term “BIA efflux transporter” as used herein refers to a superfamily of ABC transport proteins capable of moving a BIA across a cellular membrane to efflux it out of the cell. i.e. such that the concentration of said intermediate is increased outside of the host cell relative to inside the host cell. In some aspects, the one or more benzylisoquinoline alkaloid and/or BIA derivative is selected from one or more BIA, BIA-glycoside, oripavine, glucosylated oripavine, gly-oripavine, thebaine, northebaine, nororipavine, gly-nororipavine, glucosylated nororipavine, nor-opioids or glycosylated noropioids. In aspects of the current invention, ABC transport protein capable of effluxing one or more BIA, BIA-glycoside, oripavine, thebaine, northebaine, nororipavine or glycosylated nororipavine or glucosylated nororipavine from the recombinant microbial host cell, comprises a Walker A sequence G(A/S/R)(S/T)GAGK(S/T), a linker sequence (L/V)SGG(E/Q), and a Walker B sequence comprising four hydrophobic residues, an optional additional fifth hydrophobic residue and a D such that (I/L)(I/L)(I/V/L)(F/L/M)XD where X represents the optional additional hydrophobic residue or no additional residue. In some aspects, the one or more benzylisoquinoline alkaloid (such as BIA and/or BIA derivative, BIA-glycoside, oripavine, glucosylated oripavine, gly-oripavine, thebaine, northebaine, nororipavine, gly-nororipavine, glucosylated nororipavine, nor-opioids or glycosylated noropioids) efflux transporters of the current invention are ABC transporters. In many aspects, the ABC transporters of the current invention have been selected on the basis of increased specificity for the BIAs and/or BIA derivatives produced by the recombinant host cell (one or more BIA, BIA-glycoside, oripavine, glucosylated oripavine, gly-oripavine, thebaine, northebaine, nororipavine, gly-nororipavine, glucosylated nororipavine, nor-opioids or glycosylated noropioids) compared to one or more intermediate molecules in or substrates fed to the BIA-producing biosynthetic pathway engineered into the recombinant host cell. Examples of BIA efflux transporters as described herein include but are not limited to ABC transporter polypeptides comprising a sequence having at least 45%, such as at least 60%, such as at least 70%, such as at least 80%, such as at least 85%, such as at least 90%, such as at least 95%, such as at least 99%, such as 100% identity to any of SEQ ID No, 872, 910, 912, 914, 916, 918, 920, 922, 924, 926, 928, 930, 932, 934, 936, 938, 940, 942, 956, 960, 962, 964, 966, 970, 976, 980, 986, 988, 990, 994, 996, 1010, 1012, 1018, 1020, 1022, 1026, 1028, 1030, 1032, 1034, 1038, or 1040, or is encoded by a nucleic acid sequence having at least 45%, such as at least 60%, such as at least 70%, such as at least 80%, such as at least 85%, such as at least 90%, such as at least 95%, such as at least 99%, such as 100% identity to any of SEQ ID No. 871, 909, 911, 913, 915, 947, 919, 921, 923, 925, 927, 929, 931, 933, 935, 937, 939 or 941, 955, 959, 961, 963, 965, 969, 975, 979, 985, 987, 989, 993, 995, 1009, 1011, 1017, 1019, 1021, 1025, 1027, 1029, 1031, 1033, 1037, 1039 or genomic DNA thereof.

In some aspects, BIA (one or more BIA, BIA-glycoside, oripavine, glucosylated oripavine, gly-oripavine, thebaine, northebaine, nororipavine, gly-nororipavine, glucosylated nororipavine, nor-opioids or glycosylated noropioids) efflux transporters are members of the ABCC/multi-drug resistance associated protein (MRP) subfamily of ABC transporters. In some aspects, the current inventions provides recombinant microbial host cells capable of producing one or more benzylisoquinoline alkaloid (BIA, BIA-glycoside, oripavine, thebaine, northebaine, nororipavine or glycosylated nororipavine or glucosylated nororipavine), comprising BIA efflux transporters of the ABCC sub-family of ABC transporters, comprising Walker A sequences G(X)(I/V)G(S/T)GK where X is a residue selected from P, L, S, A, V or M and GRTGAGK, two linker sequences comprising LSGGQ and NFSLGE, and Walker B sequences (I/V/T)(I/Y/V)L(M/F/L)D and I(I/L)(I/V)(L/M)D. In some aspects, the current inventions provides recombinant microbial host cells capable of producing one or more benzylisoquinoline alkaloid (BIA, BIA-glycoside, oripavine, thebaine, northebaine, nororipavine or glycosylated nororipavine or glucosylated nororipavine), comprising BIA efflux transporters of the ABCC sub-family of ABC transporters, wherein the ABCC/multi-drug resistance associated protein (MRP) ABC transporter is a polypeptide comprising a sequence having at least 45%, such as at least 60%, such as at least 70%, such as at least 80%, such as at least 85%, such as at least 90%, such as at least 95%, such as at least 99%, such as 100% identity to SEQ ID No. 872, 910, 912, 914, 918, 920, 922, 924, 926, 928, 930, 932, 934, 936, 938, 940, 942, 956, 960, 962, 964, 966, 970, 1032, 1034, or 1040, or encoded by a nucleic acid sequence having at least 45%, such as at least 60%, such as at least 70%, such as at least 80%, such as at least 85%, such as at least 90%, such as at least 95%, such as at least 99%, such as 100% identity to SEQ ID No. 871, 909, 911, 913, 917, 919, 921, 923, 925, 927, 929, 931, 933, 935, 937, 939, 941, 955, 959, 961, 963, 965, 969, 1031, 1033 or 1039, or genomic DNA thereof.

In some aspects, BIA (such as BIA-glycoside, oripavine, thebaine, northebaine, nororipavine or gly-nororipavine or glucosylated nororipavine) efflux transporters are members of the members of the ABCG/pleiotropic drug resistance (PDR) subfamily of ABC transporters. In some aspects, the current inventions provides recombinant microbial host cells capable of producing BIAs of relevance to some aspects of the current invention include one or more benzylisoquinoline alkaloid (such as any BIA and/or BIA derivative, BIA-glycoside, oripavine, glucosylated oripavine, gly-oripavine, thebaine, northebaine, nororipavine, gly-nororipavine, glucosylated nororipavine, nor-opioids or glycosylated noropioids), comprising BIA efflux transporters of the ABCG/pleiotropic drug resistance (PDR) subfamily of ABC transporters comprising Walker A sequences GRPGSGC(S/T) and G(A/S)SGAGKT, S sequences VSGGERKRVSIA and LNVEQRKRLTIG, and Walker B sequences (F/L)QCWD and LL(V/L)F(L/F)D. In some aspects, the current inventions provides recombinant microbial host cells capable of producing one or more benzylisoquinoline alkaloid (BIA, BIA-glycoside, oripavine, glucosylated oripavine, gly-oripavine, thebaine, northebaine, nororipavine, gly-nororipavine, glucosylated nororipavine, nor-opioids or glycosylated noropioids), comprising BIA efflux transporters of the ABCG/pleiotropic drug resistance (PDR) subfamily of ABC transporters, wherein the ABCG (PDR) ABC transporter is a polypeptide comprising a sequence having at least 45%, such as at least 60%, such as at least 70%, such as at least 80%, such as at least 85%, such as at least 90%, such as at least 95%, such as at least 99%, such as 100% identity to a polypeptide comprising a sequence having at least 45%, such as at least 60%, such as at least 75%, such as at least 80%, such as at least 85%, such as at least 90%, such as at least 95%, such as at least 99%, such as 100% identity to SEQ ID No. 916, 976, 980, 986, 988, 990, 994, 996, 1010, 1012, 1018, 1020, 1022, 1026, 1028, 1030 or 1038, or encoded by a nucleic acid sequence having at least 70%, such as at least 80%, such as at least 85%, such as at least 90%, such as at least 95%, such as at least 99%, such as 100% identity to SEQ ID No. 915, 975, 979, 985, 987, 989, 993, 995, 1009, 1011, 1017, 1019, 1021, 1025, 1027, 1029 or 1037 or genomic DNA thereof.

The terms “substantially” or “approximately” or “about”, as used herein refers to a reasonable deviation around a value or parameter such that the value or parameter is not significantly changed. These terms of deviation from a value should be construed as including a deviation of the value where the deviation would not negate the meaning of the value deviated from. For example, in relation to a reference numerical value the terms of degree can include a range of values plus or minus 10% from that value. For example, deviation from a value can include a specified value plus or minus a certain percentage from that value, such as plus or minus 9%, 8%, 7%, 6%, 5%, 4%, 3%, 2%, or 1% from the specified value.

The term “and/or” as used herein is intended to represent an inclusive “or”. The wording X and/or Y is meant to mean both X or Y and X and Y. Further the wording X, Y and/or Z is intended to mean X, Y and Z alone or any combination of X, Y, and Z.

The term “isolated” as used herein about a compound, refers to any compound, which by means of human intervention, has been put in a form or environment that differs from the form or environment in which it is found in nature. Isolated compounds include but is no limited to compounds of the invention for which the ratio of the compounds relative to other constituents with which they are associated in nature is increased or decreased. In an important embodiment the amount of compound is increased relative to other constituents with which the compound is associated in nature. In an embodiment the compound of the invention may be isolated into a pure or substantially pure (“purified”) form. In this context a substantially pure compound means that the compound is separated from other extraneous or unwanted material present from the onset of producing the compound or generated in the manufacturing process. Such a substantially pure compound preparation contains less than 10%, such as less than 8%, such as less than 6%, such as less than 5%, such as less than 4%, such as less than 3%, such as less than 2%, such as less than 1%, such as less than 0.5% by weight of other extraneous or unwanted material usually associated with the compound when expressed natively or recombinantly. In an embodiment the isolated compound is at least 90% pure, such as at least 91% pure, such as at least 92% pure, such as at least 93% pure, such as at least 94% pure, such as at least 95% pure, such as at least 96% pure, such as at least 97% pure, such as at least 98% pure, such as at least 99% pure, such as at least 99.5% pure, such as 100% pure by weight.

The term “non-naturally occurring” as used herein about a substance, refers to any substance that is not normally found in nature or natural biological systems. In this context the term “found in nature or in natural biological systems” does not include the finding of a substance in nature resulting from releasing the substance to nature by deliberate or accidental human intervention. Non-naturally occurring substances may include substances completely or partially synthetized by human intervention and/or substances prepared by human modification of a natural substance.

The term “Sequence Identity” as used herein considers the degree of sequence similarity or relatedness between two amino acid sequences or between two nucleotide sequences. The term “% identity” is used herein as a quantifiable measure of the Sequence Identity or relatedness between two amino acid sequences or between two nucleotide sequences. More precisely, the term “% identity” as used herein about amino acid or nucleotide sequences refers to the degree of identity in percent between two amino acid sequences obtained when using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

$$\frac{identical amino acid residues}{Length of alignment - total number of gaps in alignment} \times 100$$

The term “% identity” as used herein about nucleotide sequences refers to the degree of identity in percent between two nucleotide sequences obtained when using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

$$\frac{identical deoxyribonucleotides}{Length of alignment - total number of gaps in alignment} \times 100$$

The protein sequences of the present invention can further be used as a “query sequence” to perform a search against sequence databases, for example to identify other family members or related sequences. Such searches can be performed using the BLAST programs. Software for performing BLAST analyses is publicly available through the National Center for Biotechnology Information (http://www.ncbi.nlm.nih.gov). BLASTP is used for amino acid sequences and BLASTN for nucleotide sequences. The BLAST program uses as defaults:
Cost to open gap: default=5 for nucleotides/11 for proteins
Cost to extend gap: default=2 for nucleotides/1 for proteins
Penalty for nucleotide mismatch: default=−3
Reward for nucleotide match: default=1
Expect value: default=10
Wordsize: default=11 for nucleotides/28 for megablast/3 for proteins.

Furthermore, the degree of local identity between the amino acid sequence query or nucleic acid sequence query and the retrieved homologous sequences is determined by the BLAST program. However only those sequence segments are compared that give a match above a certain threshold. Accordingly, the program calculates the identity only for these matching segments. Therefore, the identity calculated in this way is referred to as local identity. Alternatively, % identity for any candidate nucleic acid or amino acid sequence relative to a reference sequence can be determined as follows. A reference sequence (e.g., a nucleic acid sequence or an amino acid sequence described herein) is aligned to one or more candidate sequences using the computer program Clustal Omega (version 1.2.1, default parameters), which allows alignments of nucleic acid or polypeptide sequences to be carried out across their entire length (global alignment). Chenna et al., 2003, Nucleic Acids Res. 31 (13): 3497-500.

Clustal Omega calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: % age; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: % age; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gin, Glu, Arg, and Lys; residue-specific gap penalties: on. The Clustal Omega output is a sequence alignment that reflects the relationship between sequences. Clustal Omega can be run, for example, at the Baylor College of Medicine Search Launcher site on the World Wide Web (searchlauncher.bcm.tmc.edu/multi-align/multi-align.html) and at the European Bioinformatics Institute site at http://www.ebi.ac.uk/Tools/msa/clustalo/. To determine a % identity of a candidate nucleic acid or amino acid sequence to a reference sequence, the sequences are aligned using Clustal Omega, the number of identical matches in the alignment is divided by the length of the reference sequence, and the result is multiplied by 100. It is noted that the % identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

The term “mature polypeptide” or “mature enzyme” as used herein refers to a polypeptide in its final active form following translation and any post-translational modifications, such as N-terminal processing, C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide.

The term “cDNA” refers to a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

The term “coding sequence” refers to a nucleotide sequence, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

The term “control sequence” as used herein refers to a nucleotide sequence necessary for expression of a polynucleotide encoding a polypeptide. A control sequence may be native (i.e., from the same gene) or heterologous or foreign (i.e., from a different gene) to the polynucleotide encoding the polypeptide. Control sequences include, but are not limited to leader sequences, polyadenylation sequence, pro-peptide coding sequence, promoter sequences, signal peptide coding sequence, translation terminator (stop) sequences and transcription terminator (stop) sequences. To be operational control sequences usually must include promoter sequences, transcriptional and translational stop signals. Control sequences may be provided with linkers for the purpose of introducing specific restriction sites facilitating ligation of the control sequences with a coding region of a polynucleotide encoding a polypeptide.

The term “expression” includes any step involved in the production of a polypeptide including, but not limited to, transcription, post-transcriptional modification, translation, post-translational modification, and secretion.

The term “expression vector” refers to a DNA molecule, either single- or double stranded, either linear or circular, which comprises a polynucleotide encoding a polypeptide and is operably linked to control sequences that provide for its expression. Expression vectors include expression cassettes for the integration of genes into a host cell as well as plasmids and/or chromosomes comprising such genes.

The term “host cell” refers to any cell type that is susceptible to transformation, transfection, transduction, or the like with a nucleic acid construct or expression vector comprising a polynucleotide of the present invention. Host cell encompasses any progeny of a parent cell that is not identical to the parent cell due to mutations that occur during replication.

The term “polynucleotide construct” refers to a polynucleotide, either single- or double stranded, which is isolated from a naturally occurring gene or is modified to contain segments of nucleic acids in a manner that would not otherwise exist in nature or which is synthetic, and which comprises a polynucleotide encoding a polypeptide and one or more control sequences.

The term “operably linked” refers to a configuration in which a control sequence is placed at an appropriate position relative to the coding polynucleotide such that the control sequence directs expression of the coding polynucleotide.

The terms “nucleotide sequence and “polynucleotide” are used herein interchangeably.

The term “comprise” and “include” as used throughout the specification and the accompanying items as well as variations such as “comprises”, “comprising”, “includes” and “including” are to be interpreted inclusively. These words are intended to convey the possible inclusion of other elements or integers not specifically recited, where the context allows.

The articles “a” and “an” are used herein refers to one or to more than one (i.e. to one or at least one) of the grammatical object of the article. By way of example, “an element” may mean one element or more than one element.

Terms like “preferably”, “commonly”, “particularly”, and “typically” are not utilized herein to limit the scope of the itemed invention or to imply that certain features are critical, essential, or even important to the structure or function of the itemed invention. Rather, these terms are merely intended to highlight alternative or additional features that can or cannot be utilized in a particular embodiment of the present invention.

The term “cell culture” as used herein refers to a culture medium comprising a plurality of host cells of the invention. A cell culture may comprise a single strain of host cells or may comprise two or more distinct host cell strains. The culture medium may be any medium that may comprise a recombinant host, e.g., a liquid medium (i.e., a culture broth) or a semi-solid medium, and may comprise additional components, e.g., a carbon source such as dextrose, sucrose, glycerol, or acetate; a nitrogen source such as ammonium sulfate, urea, or amino acids; a phosphate source; vitamins; trace elements; salts; amino acids; nucleobases; yeast extract; aminoglycoside antibiotics such as G418 and hygromycin B.

All methods described herein can be performed in any suitable order of steps unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

All percentages, ratios and proportions herein are by weight, unless otherwise specified. A weight percent (weight %, also as wt. %) of a component, unless specifically stated to the contrary, is based on the total weight of the composition in which the component is included (e.g., on the total amount of the reaction mixture).

Terms used herein may be preceded and/or followed by a single dash, “ ”, or a double dash, “=”, to indicate the bond order of the bond between the named substituent and its parent moiety; a single dash indicates a single bond and a double dash indicates a double bond or a pair of single bonds in the case of a spiro-substituent. In the absence of a single or double dash it is understood that a single bond is formed between the substituent and its parent moiety; further, substituents are intended to be read “left to right” with reference to the chemical structure referred to unless a dash indicates otherwise. For example, arylalkyl, arylalkyl-, and alkylaryl indicate the same functionality.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety can refer to a monovalent radical (e.g. CH3-CH2-), in some circumstances a bivalent linking moiety can be “alkyl,” in which case those skilled in the art will understand the alkyl to be a divalent radical (e.g., —CH2-CH2-), which is equivalent to the term “alkylene.” (Similarly, in circumstances in which a divalent moiety is required and is stated as being “aryl,” those skilled in the art will understand that the term “aryl” refers to the corresponding divalent moiety, arylene). All atoms are understood to have their normal number of valences for bond formation (i.e., 4 for carbon, 3 for N, 2 for O, and 2, 4, or 6 for S, depending on the oxidation state of the S). Nitrogens in the presently disclosed compounds can be hypervalent, e.g., an N-oxide or tetrasubstituted ammonium salt. On occasion a moiety may be defined, for example, as —B-(A)a, wherein a is 0 or 1. In such instances, when a is 0 the moiety is —B and when a is 1 the moiety is —B-A.

As used herein, the term “alkyl” or “alkane” includes a saturated hydrocarbon having a designed number of carbon atoms, such as 1 to 40 carbons (i.e., inclusive of 1 and 40), 1 to 35 carbons, 1 to 25 carbons, 1 to 20 carbons, or 1, 2, 3, 4, 5, 6, 7, 8, 9, 10, 11, 12, 13, 14, 15, 16, 17, or 18. Alkyl groups or alkanes may be straight or branched and depending on context, may be a monovalent radical or a divalent radical (i.e., an alkylene group). For example, the moiety “—(C1 C6 alkyl)O—” signifies connection of an oxygen through an alkylene bridge having from 1 to 6 carbons and C1-C3 alkyl represents methyl, ethyl, and propyl moieties. Examples of “alkyl” include, for example, methyl, ethyl, propyl, isopropyl, butyl, iso, sec and tert butyl, pentyl, and hexyl. Examples of “alkane” include, for example, methane, ethane, propane, isopropane, butane, isobutane, sec-butane, tert-butane, pentane, hexane, heptane, and octane.

The term “alkoxy” represents an alkyl group of indicated number of carbon atoms attached to the parent molecular moiety through an oxygen bridge. Examples of “alkoxy” include, for example, methoxy, ethoxy, propoxy, and isopropoxy.

The term “alkenyl” as used herein, unsaturated hydrocarbon containing from 2 to 10 carbons (i.e., inclusive of 2 and 10), 2 to 8 carbons, 2 to 6 carbons, or 2, 3, 4, 5 or 6, unless otherwise specified, and containing at least one carbon-carbon double bond. Alkenyl group may be straight or branched and depending on context, may be a monovalent radical or a divalent radical (i.e., an alkenylene group). For example, the moiety “—(C2 C6 alkenyl)O—” signifies connection of an oxygen through an alkenylene bridge having from 2 to 6 carbons. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

The term “alkynyl” as used herein, unsaturated hydrocarbon containing from 2 to 10 carbons (i.e., inclusive of 2 and 10), 2 to 8 carbons, 2 to 6 carbons, or 2, 3, 4, 5 or 6 unless otherwise specified, and containing at least one carbon-carbon triple bond. Alkynyl group may be straight or branched and depending on context, may be a monovalent radical or a divalent radical (i.e., an alkynylene group). For example, the moiety “—(C2 C6 alkynyl)O—” signifies connection of an oxygen through an alkynylene bridge having from 2 to 6 carbons. Representative examples of alkynyl include, but are not limited to, acetylenyl, 1-propynyl, 2-propynyl, 3-butynyl, 2-pentynyl, and 1-butynyl.

The term “aryl” represents an aromatic ring system having a single ring (e.g., phenyl) which is optionally fused to other aromatic hydrocarbon rings or non-aromatic hydrocarbon or heterocyclic rings. “Aryl” includes ring systems having multiple condensed rings and in which at least one is carbocyclic and aromatic, (e.g., 1,2,3,4 tetrahydronaphthyl, naphthyl). Examples of aryl groups include phenyl, 1 naphthyl, 2 naphthyl, indanyl, indenyl, dihydronaphthyl, fluorenyl, tetralinyl, and 6,7,8,9-tetrahydro-5H-benzo[a]cycloheptenyl. “Aryl” also includes ring systems having a first carbocyclic, aromatic ring fused to a nonaromatic heterocycle, for example, 1H-2,3 dihydrobenzofuranyl and tetrahydroisoquinolinyl. The aryl groups herein are unsubstituted or, when specified as “optionally substituted”, can unless stated otherwise be substituted in one or more substitutable positions with various groups as indicated.

The term “heteroaryl” refers to an aromatic ring system containing at least one aromatic heteroatom selected from nitrogen, oxygen and sulfur in an aromatic ring. Most commonly, the heteroaryl groups will have 1, 2, 3, or 4 heteroatoms. The heteroaryl may be fused to one or more non-aromatic rings, for example, cycloalkyl or heterocycloalkyl rings, wherein the cycloalkyl and heterocycloalkyl rings are described herein. In one embodiment of the present compounds the heteroaryl group is bonded to the remainder of the structure through an atom in a heteroaryl group aromatic ring. In another embodiment, the heteroaryl group is bonded to the remainder of the structure through a non-aromatic ring atom. Examples of heteroaryl groups include, for example, pyridyl, pyrimidinyl, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, benzo[1,4]oxazinyl, triazolyl, tetrazolyl, isothiazolyl, naphthyridinyl, isochromanyl, chromanyl, isoindolinyl, isobenzothienyl, benzoxazolyl, pyridopyridinyl, purinyl, benzodioxolyl, triazinyl, pteridinyl, benzothiazolyl, imidazopyridinyl, imidazothiazolyl, benzisoxazinyl, benzoxazinyl, benzopyranyl, benzothiopyranyl, chromonyl, chromanonyl, pyridinyl N-oxide, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S oxide, benzothiopyranyl S,S dioxide. Preferred heteroaryl groups include pyridyl, pyrimidyl, quinolinyl, indolyl, pyrrolyl, furanyl, thienyl and imidazolyl, pyrazolyl, indazolyl, thiazolyl and benzothiazolyl. In certain embodiments, each heteroaryl is selected from pyridyl, pyrimidinyl, pyridazinyl, pyrazinyl, imidazolyl, isoxazolyl, pyrazolyl, oxazolyl, thiazolyl, furanyl, thienyl, pyrrolyl, oxadiazolyl, thiadiazolyl, triazolyl, tetrazolyl, isothiazolyl, pyridinyl N-oxide, pyrrolyl N-oxide, pyrimidinyl N-oxide, pyridazinyl N-oxide, pyrazinyl N-oxide, imidazolyl N-oxide, isoxazolyl N-oxide, oxazolyl N-oxide, thiazolyl N-oxide, pyrrolyl N-oxide, oxadiazolyl N-oxide, thiadiazolyl N-oxide, triazolyl N-oxide, and tetrazolyl N-oxide. Preferred heteroaryl groups include pyridyl, pyrimidyl, quinolinyl, indolyl, pyrrolyl, furanyl, thienyl, imidazolyl, pyrazolyl, indazolyl, thiazolyl and benzothiazolyl. The heteroaryl groups herein are unsubstituted or, when specified as “optionally substituted”, can unless stated otherwise be substituted in one or more substitutable positions with various groups, as indicated.

The term “heterocycloalkyl” refers to a non-aromatic ring or ring system containing at least one heteroatom that is preferably selected from nitrogen, oxygen and sulfur, wherein said heteroatom is in a non-aromatic ring. The heterocycloalkyl may have 1, 2, 3 or 4 heteroatoms. The heterocycloalkyl may be saturated (i.e., a heterocycloalkyl) or partially unsaturated (i.e., a heterocycloalkenyl). Heterocycloalkyl includes monocyclic groups of three to eight annular atoms as well as bicyclic and polycyclic ring systems, including bridged and fused systems, wherein each ring includes three to eight annular atoms. The heterocycloalkyl ring is optionally fused to other heterocycloalkyl rings and/or non-aromatic hydrocarbon rings. In certain embodiments, the heterocycloalkyl groups have from 3 to 7 members in a single ring. In other embodiments, heterocycloalkyl groups have 5 or 6 members in a single ring. In some embodiments, the heterocycloalkyl groups have 3, 4, 5, 6 or 7 members in a single ring. Examples of heterocycloalkyl groups include, for example, azabicyclo[2.2.2]octyl (in each case also “quinuclidinyl” or a quinuclidine derivative), azabicyclo[3.2.1]octyl, 2,5-diazabicyclo[2.2.1]heptyl, morpholinyl, thiomorpholinyl, thiomorpholinyl S oxide, thiomorpholinyl S,S dioxide, 2 oxazolidonyl, piperazinyl, homopiperazinyl, piperazinonyl, pyrrolidinyl, azepanyl, azetidinyl, pyrrolinyl, tetrahydropyranyl, piperidinyl, tetrahydrofuranyl, tetrahydrothienyl, 3,4-dihydroisoquinolin-2(1H)-yl, isoindolindionyl, homopiperidinyl, homomorpholinyl, homothiomorpholinyl, homothiomorpholinyl S,S dioxide, oxazolidinonyl, dihydropyrazolyl, dihydropyrrolyl, dihydropyrazinyl, dihydropyridinyl, dihydropyrimidinyl, dihydrofuryl, dihydropyranyl, imidazolidonyl, tetrahydrothienyl S oxide, tetrahydrothienyl S,S dioxide and homothiomorpholinyl S oxide. Especially desirable heterocycloalkyl groups include morpholinyl, 3,4-dihydroisoquinolin-2(1H)-yl, tetrahydropyranyl, piperidinyl, aza bicyclo[2.2.2]octyl, γ butyrolactonyl (i.e., an oxo substituted tetrahydrofuranyl), γ butryolactamyl (i.e., an oxo substituted pyrrolidine), pyrrolidinyl, piperazinyl, azepanyl, azetidinyl, thiomorpholinyl, thiomorpholinyl S,S dioxide, 2 oxazolidonyl, imidazolidonyl, isoindolindionyl, piperazinonyl. The heterocycloalkyl groups herein are unsubstituted or, when specified as “optionally substituted”, can unless stated otherwise be substituted in one or more substitutable positions with various groups, as indicated.

The term “cycloalkyl” or “cycloalkane” refers to a non-aromatic carbocyclic ring or ring system, which may be saturated (i.e., a cycloalkyl, a cycloalkane) or partially unsaturated (i.e., a cycloalkenyl). The cycloalkyl ring can be optionally fused to or otherwise attached (e.g., bridged systems) to other cycloalkyl rings. Certain examples of cycloalkyl groups or cycloalkanes present in the disclosed compounds have from 3 to 7 members in a single ring, such as having 5 or 6 members in a single ring. In some embodiments, the cycloalkyl groups have 3, 4, 5, 6 or 7 members in a single ring. Examples of cycloalkyl groups include, for example, cyclohexyl, cyclopentyl, cyclobutyl, cyclopropyl, tetrahydronaphthyl and bicyclo[2.2.1]heptane. Examples of cycloalkanes include, for example, cyclohexane, methylcyclohexane, cyclohexanone, cyclohexanol, cyclopentane, cycloheptane, and cycloctane. The cycloalkyl groups herein are unsubstituted or, when specified as “optionally substituted”, may be substituted in one or more substitutable positions with various groups, as indicated.

The term “ring system” encompasses monocycles, as well as fused and/or bridged polycycles.

The terms “halogen” or “halo” indicate fluorine, chlorine, bromine, and iodine. In certain embodiments of each and every embodiment described herein, the term “halogen” or “halo” refers to fluorine or chlorine. In certain embodiments of each and every embodiment described herein, the term “halogen” or “halo” refers to fluorine.

The term “halide” indicates fluoride, chloride, bromide, and iodide. In certain embodiments of each and every embodiment described herein, the term “halide” refers to bromide or chloride.

The term “substituted,” when used to modify a specified group or radical, means that one or more hydrogen atoms of the specified group or radical are each, independently of one another, replaced with the same or different substituent groups as defined below, unless specified otherwise.

Optionally, the BIAs produced herein may be converted chemically or enzymatically into BIA derivatives. For example, nororipavine and glycosylated noripavine are suitable raw materials for chemical synthesis of many useful compounds such as NaI-BIAs. Chemical production of BIA-intermediates and BIAs is taught in, for example, WO 2018/211331 and WO 2021/144362. Various NaI-opioids synthesized from nororipavine have previously been taught in A. Sipos, S. Berenyi and S. Antus. Helvetica Chimica Acta Vol. 92 (2009) pp 1359-1365.

### Genetically Modified Host Cells

Recombinant microorganisms optimized to produce benzylisoquinoline alkaloids (BIA, BIA-glycoside, oripavine, thebaine, northebaine, nororipavine or glycosylated nororipavine or glucosylated nororipavine) are in great need and even more so host cells optimized to demethylate benzylisoquinoline alkaloids such as thebaine and/or oripavine into the corresponding northebaine and/or nororipavine, which are in high demand for chemical conversion into other pharmaceutically relevant benzylisoquinoline alkaloids (BIAs).

The invention provides in a first aspect such a genetically modified (recombinant) microbial host cell capable of producing one or more BIA (benzylisoquinoline alkaloids including but not limited to any BIA, BIA-glycoside, oripavine, glucosylated oripavine, gly-oripavine, thebaine, northebaine, nororipavine, gly-nororipavine, glucosylated nororipavine, nor-opioids or glycosylated noropioids) comprising a pathway having enhanced production of one or more benzylisoquinoline alkaloids wherein the cell comprises:
(1) one or more heterologous CYP demethylases capable of converting thebaine into northebaine, thebaine into oripavine, northebaine into nororipavine and/or oripavine into nororipavine, and one or more demethylase cytochrome P450 reductase (demethylase-CPR), and/or
(2) heterologous sequences encoding:
   (a) a tyrosine hydroxylase (TH) converting L-tyrosine into L-dopa, and
   (b) optionally, a TH-CPR capable of reducing the TH of i), and
   (c) a L-dopa decarboxylase (DODC) converting L-dopa into dopamine, or a tyrosine decarboxylase (TYDC) converting L-dopa into dopamine, and
   (d) a hydroxyphenylpyruvate decarboxylase (HPPDC) converting 4-HPP into 4-HPAA, and
   (e) a monoamine oxidase converting dopamine into 3,4-DHPAA, or a N-methyl-coclaurine hydroxylase (NMCH) converting (S)-Coclaurine into (S)-3′-hydroxycoclaurine and/or (S)—N-Methylcoclaurine into (S)-3′-Hydroxy-N-Methylcoclaurine; and
   (f) a norcoclaurine synthase (NCS) converting Dopamine and 4-HPAA into (S)-norcoclaurine and/or 3,4-DHPAA and dopamine to NLDS, and
   (g) a 6-O-methyltransferase (6-OMT) converting (S)-norcoclaurine into (S)-Coclaurine and/or norlaudanosoline into (S)-3′-Hydroxy-coclaurine, and
   (h) a coclaurine-N-methyltransferase (CNMT) converting (S)-Coclaurine into (S)—N-Methylcoclaurine and/or (S)-3′-hydroxycoclaurine into (S)-3′-hydroxy-N-methyl-coclaurine, and
   (i) a N-methyl-coclaurine hydroxylase (NMCH) converting (S)-Coclaurine into (S)-3′-hydroxycoclaurine and/or (S)—N-Methylcoclaurine into (S)-3′-Hydroxy-N-Methylcoclaurine, and
   (j) a 3′-hydroxy-N-methyl-(S)-coclaurine 4′-O-methyltransferase (4′-OMT) converting (S)-3′-Hydroxy-N-Methylcoclaurine into (S)-reticuline, and
   (k) a 1,2-dehydroreticuline synthase-1,2-dehydroreticuline reductase (DRS-DRR) converting (S)-reticuline into (R)-reticuline comprised of one or more proteins, and
   (l) a salutaridine synthase (SAS) converting (R)-reticuline into Salutaridine, and
   (m) a salutaridine reductase (SAR) converting Salutaridine to Salutaridinol, and
   (n) a salutaridinol 7-O-acetyltransferase (SAT) converting Salutaridinol into 7-O-acetylsalutaridinol, and
   (o) a thebaine synthase (THS) converting 7-O-acetylsalutaridinol or 7-O-acetylsalutaridinol acetate into thebaine;
   (p) a demethylase converting thebaine into oripavine, thebaine into northebaine, oripavine into nororipavine and/or northebaine into nororipavine; and/or
   (q) a demethylase-CPR capable of reducing the demethylase of xvi).
(3) and optionally, one or more glycosyl transferases capable of transferring a glycosyl moiety to oripavine or nororipavine.

In some aspects, the pathway from tyrosine to thebaine (and thence to downstream BIAs) in the recombinant microbial host cell comprising a BIA-efflux transporter comprises the enzyme activities: TYRH, DODC, NCS, 6OMT, CNMT, NMCH, 4OMT, DRS-DRR, SAS, SAR, SAT, THS and if the BIA is downstream of thebaine, optionally a demethylase converting thebaine into oripavine, thebaine into northebaine, oripavine into nororipavine and/or northebaine into nororipavine, and optionally a demethylase-CPR capable of reducing the demethylase, optionally, one or more glycosyl transferases capable of transferring a glycosyl moiety to oripavine or nororipavine. In other aspects, the pathway from tyrosine to thebaine (and thence to downstream BIAs) in the recombinant microbial host cell comprising a BIA-efflux transporter comprises the enzyme activities: TYRH, DODC, MAO, NCS, 6OMT, CNMT, 4 OMT, DRS-DRR, sal synthase, sal reductase, sat1, thebaine synthase.

In some aspects, the pathway from tyrosine to thebaine (and thence to downstream BIAs) in the recombinant microbial host cell comprising a BIA-efflux transporter comprises one or more selected from:
a) expression of one or more heterologous genes encoding a demethylase capable of converting thebaine into northebaine, thebaine into oripavine, northebaine into nororipavine and/or oripavine into nororipavine;
b) expression of one or more heterologous genes encoding a tyrosine hydroxylases (TH) converting L-tyrosine into L-dopa selected from TH's having at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the TH comprised in 7, 9, 11, 13, 15, 17, 19, 21, 23, 25, 27, 29, 31, 33, 35, 37, 39, 41, 43, 45, 47, 49, 51, 53, 55, 57, 59, 61, 63 or 65;
c) reduction or elimination of activity of one or more dehydrogenases native to the host cell selected from the dehydrogenases comprised in SEQ ID NO: 663, 665, 667, 669, 671, 673, 675, 677, 679, 681, 683, 685, 687, 689, 691, 693, 695, 697, 699, 701, 703 or 705;
d) reduction or elimination of activity of one or more reductases native to the host cell selected from the reductases comprised in SEQ ID NO: 707, 709, 711, 713, 715, 717, 719, 721, 723, 725, 727, 729 or 731;
e) expression of one or more heterologous genes encoding a norcoclaurine synthases (NCS) converting Dopamine and 4-HPAA into (S)-norcoclaurine selected from NCS's having at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the NCS comprised in SEQ ID NO: 73 OR 76;
f) expression of one or more heterologous genes encoding
   i) a fused 1,2-dehydroreticuline synthase-1,2-dehydroreticuline reductases (DRS-DRR) converting (S)-Reticuline into (R)-reticuline, wherein
      ia) the DRS-DDRs has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DRS-DRR comprised in SEQ ID NO: 92, 94, 96; or
      ib) the DRS moiety has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DRS comprised in SEQ ID NO: 98, 100, 102, 104 or 106; and the DRR moiety has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DRR comprised in SEQ ID NO: 108 or 110; or
   ii) a DRS having at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DRS comprised in SEQ ID NO: 98, 100, 102, 104 or 106; and a DRR having at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DRR comprised in SEQ ID NO: 108 or 110;
g) expression of one or more heterologous genes encoding a thebaine synthase (THS) converting 7-O-acetylsalutaridinol into thebaine selected from THS's having at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the THS comprised in SEQ ID NO: 126, 127, 128, 129, 131, 133, 134, 136, 138;
h) expression of one or more heterologous genes encoding a transporter protein capable of increasing uptake in the host cell of a reticuline derivative selected from transporter proteins having at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the transporter protein comprised in SEQ ID NO: 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 733, 735, 774, 776, 778, 780, 782, 784, 786, 788, 790, 792, 794, 795, 797, 799, 801, 803, 805, 807, 809, 811, 813, 815, 817, 819, 821, 823 or 825, and
i) a recombinant polynucleotide comprising a promoter operably linked to an ABC transporter, wherein the ABC transporter is a member of the ABCG/pleiotropic drug resistance (PDR) subfamily of ABC transporters or the ABCC/multi-drug resistance associated protein (MRP) subfamily of ABC transporters, and wherein the ABC transporter is capable of effluxing from the host cell one or more opioids selected from a BIA, BIA-glycoside, oripavine, thebaine, northebaine, nororipavine or gly-nororipavine or glucosylated nororipavine.

### Heterologous Demethylase

In one aspect, the genetically modified host cells of the invention expresses, alone or in combination with other heterologous genes of the invention, one or more heterologous genes encoding one or more demethylases capable of converting thebaine into northebaine, thebaine into oripavine, northebaine into nororipavine and/or oripavine into nororipavine. The demethylase of the invention can be any suitable demethylase capable of converting thebaine into northebaine, thebaine into oripavine, northebaine into nororipavine and/or oripavine into nororipavine, which is heterologous to the host cell and which cooperates well with the other enzymes of the benzylisoquinoline alkaloid pathway and/or the auxiliary cellular mechanisms.

In a particular embodiment the demethylase have specificity towards producing the nor-compounds and produces less by-products. It has been identified that in particular insect demethylase, when expressed in a genetically modified host cell possess a hitherto unprecedented high product specificity producing a high product: by-product ratio, where the product: by-product is either (northebaine):(thebaine N-oxide), (northebaine):(northebaine oxaziridine), (nororipavine):(oripavine N-oxide) and/or (nororipavine):(nororipavine oxaziridine). Aside from more effectively converting more thebaine and/or oripavine into the desired corresponding nor-compounds, for in vivo conversion the insect demethylase of the invention also produces less N-oxide or oxaziridine by-products and this property provide advantage over the art, since such by-products may impact negatively of the cell function as well as they may interfere with efficiency of any subsequent chemical conversion steps and lower the efficiency of production. Accordingly, in one embodiment the demethylase of the invention have a product: by-product molar ratio of at least 2.0, such as at least 2.25, such as at least 2.5, such as at least 2.75, such as at least 3.0, such as at least 3.25, such as at least 3.5, such as at least 3.75, such as at least 4.0, such as at least 4.5, such as at least 5.0, such as at least 10.0, such as at least 25, such as at least 50, such as at least 75, such as at least 100 and wherein when the product is northebaine then the by-product is thebaine N-oxide and/or northebaine oxaziridine and when the product is nororipavine then the by-product is oripavine N-oxide and/or nororipavine oxaziridine.

For example, one insect demethylase of the invention remarkably displays N-demethylation activity and/or O-activity, whereby it is capable of converting thebaine of the formula I into northebaine of the formula II:

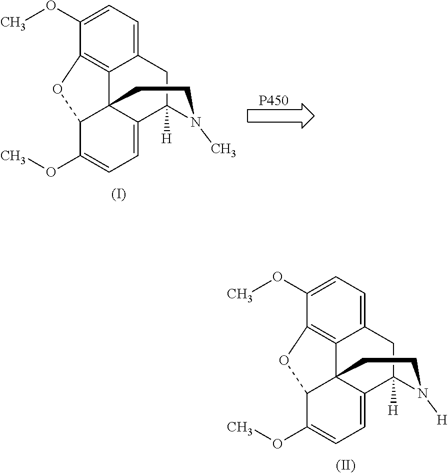

converting thebaine of the formula I into oripavine of the formula (III)

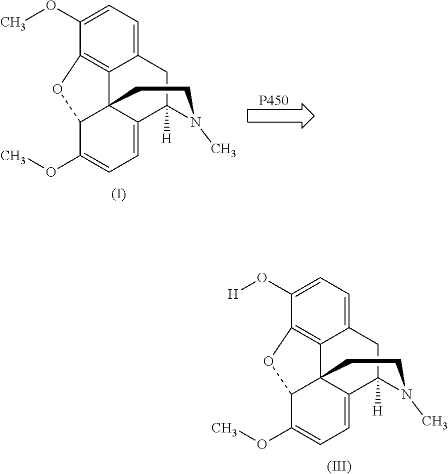

and/or converting oripavine of the formula (III) into nororipavine of formula IV:

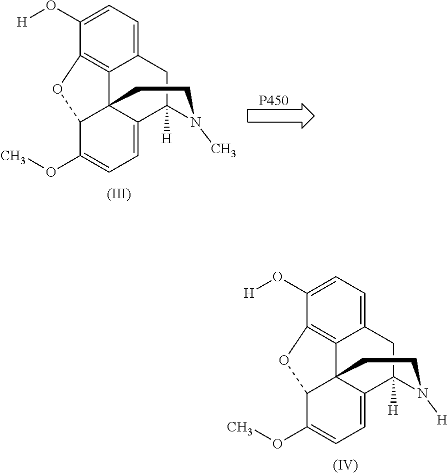

Further, the present inventors have found that demethylases derived from insects and in particular demethylases of family CYP6, are remarkably effective in converting thebaine and/or oripavine into the corresponding nor-compounds producing less by-products. Therefore, in one embodiment the demethylase of the invention is derived from an insect and in another embodiment the demethylase of the invention is of family CYP6. Relevant insects include those which feeds on plants with high contents of thebaine and/or oripavine such as poppy and include moths of the order Lepidoptera, such as moths of the genus Helicoverpa, Spodoptera, Cnaphalocrocis, Bombyx and Heliothis. Demethylases from the species Helicoverpa armigera, Spodoptera exigua, Cnaphalocrocis medinalis, Bombyx mandarina and Heliothis virescens, are particularly useful. Without being bound to the theory the present inventors contemplate that insects feeding from plants containing a high level of thebaine and/or oripavine, as a protection mechanism, during evolution have developed enzymes converting these potentially harmful substrates.

Examples of insect demethylases which works remarkably well in converting thebaine and/or oripavine with low formation of by-products in a heterologous host cell includes the demethylases selected from of SEQ ID NO: 140, 142, 144, 146, 148, 150, 152, 154, 156, 158, 160, 162, 164, 166, 168, 170, 172, 174, 176, 178, 180, 182, 184, 186, 188, 190, 192, 194, 196, 827, 829, 831, 833, 835, 837, 839, 841, 843, 845, 847, 849, 851, 853, 855, 857, 859, 861, 863, 865, 867 and 869.

Accordingly, in a further embodiment the demethylase of the invention comprises a polypeptide selected from the group consisting of:
a) a demethylase which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the demethylase comprised in any one of SEQ ID NO: 140, 142, 144, 146, 148, 150, 152, 154, 156, 158, 160, 162, 164, 166, 168, 170, 172, 174, 176, 178, 180, 182, 184, 186, 188, 190, 192, 194, 196, 827, 829, 831, 833, 835, 837, 839, 841, 843, 845, 847, 849, 851, 853, 855, 857, 859, 861, 863, 865, 867, 876 and 869;
b) a demethylase encoded by a polynucleotide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to a polynucleotide comprised in any one of SEQ ID NO: 141, 143, 145, 147, 149, 151, 153, 155, 157, 159, 161, 163, 165, 167, 169, 171, 173, 175, 177, 179, 181, 183, 185, 187, 189, 191, 193, 195, 197, 828, 830, 832, 834, 836, 838, 840, 842, 844, 846, 848, 850, 852, 854, 856, 858, 860, 862, 864, 866, 868, 875 and 870 or genomic DNA thereof; and
c) a functional variant of the demethylase of (a) or (b) capable of converting thebaine into northebaine, thebaine into oripavine, northebaine into nororipavine and/or oripavine into nororipavine.

In particular the insect demethylase is:
a) a demethylase comprised in any one of SEQ ID NO: 140, 142, 144, 146, 148, 150, 152, 154, 156, 158, 160, 162, 164, 166, 168, 170, 172, 174, 176, 178, 180, 182, 184, 186, 188, 190, 192, 194, 196, 827, 829, 831, 833, 835, 837, 839, 841, 843, 845, 847, 849, 851, 853, 855, 857, 859, 861, 863, 865, 867, 876 and 869; or
b) a demethylase encoded by a polynucleotide comprised in any one of SEQ ID NO: or genomic DNA thereof encoding the P450 comprised in any one of SEQ ID NO: 141, 143, 145, 147, 149, 151, 153, 155, 157, 159, 161, 163, 165, 167, 169, 171, 173, 175, 177, 179, 181, 183, 185, 187, 189, 191, 193, 195, 197, 828, 830, 832, 834, 836, 838, 840, 842, 844, 846, 848, 850, 852, 854, 856, 858, 860, 862, 864, 866, 868, 875 and 870.

Alternatively, the demethylase of the invention can be derived from a fungus, in particular fungi of a genus selected from Rhizopus, Lichtheimia, Syncephalastrum, Cunninghamella, Mucor, Parasitella, Absidia, Choanephora, Bifiguratus and Choanephora. In a more specific embodiment the P450 may be derived from a fungal species selected from Rhizopus microspores, Rhizopus azygosporus, Rhizopus stolonifera, Rhizopus oryzae, Rhizopus delemar, Lichtheimia corymbifera, Lichtheimia ramosa, Syncephalastrum racemosum, Cunninghamella echinulate, Mucor circinelloides, Mucor ambiguous, Parasitella parasitica, Absidia repens, Absidia glauca, Choanephora cucurbitarum, Bifiguratus adelaidae and Choanephora cucurbitarum.

Examples of fungal demethylases which works well in converting thebaine and/or oripavine with low formation of by-products in a heterologous host cell includes the demethylase selected from SEQ ID NO: 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288 or 290. Accordingly, in a further embodiment the demethylase of the invention comprises:
a) a polypeptide having at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the demethylase comprised in any one of SEQ ID NO: 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288 or 290; or
b) a polypeptide encoded by a polynucleotide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the polynucleotide comprised in any one of SEQ ID NO: 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291 or genomic DNA thereof; or
c) a functional variant of the demethylase of (a) or (b) capable of converting thebaine into northebaine, thebaine into oripavine, northebaine into nororipavine and/or oripavine into nororipavine.

In particular the fungal demethylase is:
a) the demethylase comprised in any one of SEQ ID NO: 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288 and 290; or
b) the demethylase encoded by a polynucleotide comprised in any one of SEQ ID NO: 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289 and 291 or genomic DNA thereof.

A particular demethylase of the invention is one which does not comprise one or more of the amino acids selected from:
a) Valine at a position corresponding to V75 of SEQ ID NO: 290;
b) Isoleucine at a position corresponding to I79 of SEQ ID NO: 290;
c) Isoleucine at a position corresponding to V83 of SEQ ID NO: 290;
d) Asparagine at a position corresponding to N84 of SEQ ID NO: 290;
e) Arginine at a position corresponding to R86 of SEQ ID NO: 290;
f) Aspartic acid at a position corresponding to D87 of SEQ ID NO: 290;
g) Glutamic acid at a position corresponding to E126 of SEQ ID NO: 290;
h) Threonine at a position corresponding to T145 of SEQ ID NO: 290;
i) Asparagine at a position corresponding to N172 of SEQ ID NO: 290;
j) Threonine at a position corresponding to T193 of SEQ ID NO: 290;
k) Glycine at a position corresponding to G218 of SEQ ID NO: 290;
l) Isoleucine at a position corresponding to 1236 of SEQ ID NO: 290;
m) Alanine at a position corresponding to A258 of SEQ ID NO: 290;
n) Methionine at a position corresponding to M259 of SEQ ID NO: 290;
o) Aspartic acid at a position corresponding to D298 of SEQ ID NO: 290;
p) Leucine at a position corresponding to L430 of SEQ ID NO: 290;
q) Histidine at a position corresponding to H448 of SEQ ID NO: 290;
r) Asparagine at a position corresponding to N503 of SEQ ID NO: 290;
s) Proline at a position corresponding to P506 of SEQ ID NO: 290;
t) Phenylalanine at a position corresponding to F507 of SEQ ID NO: 290;
u) Asparagine at a position corresponding to N508 of SEQ ID NO: 290; and
v) Valine at a position corresponding to V509 of SEQ ID NO: 290;

Further to this embodiment the demethylase may not comprise histidine at a position corresponding to H448 of SEQ ID NO: 290, asparagine at a position corresponding to H508 of SEQ ID NO: 290 and/or valine at a position corresponding to H509 of SEQ ID NO: 290. Still further to this embodiment the demethylase may comprise comprise tyrosine at the position corresponding to position 448 of SEQ ID NO: 290, threonine at the position corresponding to position corresponding to H508 of SEQ ID NO: 290 and/or glycine at the position corresponding to position corresponding to H509 of SEQ ID NO: 290. Within this embodiment the demethylase may specifically be the P450 of SEQ ID NO: 250 or SEQ ID NO: 252.

The demethylase of SEQ ID NO: 218, 220, 222, 224, 226, 228, 236, 240, 250, 252, 254 and 268 have in addition to N-demethylase activity also O-demethylase activity (ODM) and are capable of demethylating thebaine of the formula I into oripavine of the formula III as described supra.

In a separate embodiment the cell of the invention further comprises a demethylase-CPR capable of reducing and/or regenerating the demethylase enzyme. The demethylase-CPR may also be heterologous to the cell.

Some demethylases may work better together with a demethylase-CPR from a related source so in a particular embodiment where the demethylase is an insect demethylase, the demethylase-CPR may also advantageously be an insect demethylase-CPR, such as a demethylase-CPR derived from an insect of the order Lepidoptera, such as the insect demethylase-CPR derived from an insect of the genus helicoverpa, Heliothis or Spodoptera such as demethylase-CPR derived from an insect of the species Helicoverpa armigera, Heliothis virescens or Spodoptera exigua.

In particular, the insect demethylase-CPR may comprise a polypeptide selected from the group consisting of:
a) a demethylase-CPR which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the demethylase-CPR comprised in SEQ ID NO: 292, 294, 296, 298, 300 or 302;
b) a demethylase-CPR encoded by a polynucleotide which is at least 20% identical to the polynucleotide comprised in SEQ ID NO: 293, 295, 297, 299, 301, 303 or 305 or genomic DNA thereof; and
c) a functional variant of the demethylase-CPR of (a) or (b) capable of reducing/regenerating the demethylase of the invention.

In another embodiment where the demethylase is a fungal demethylase the demethylase-CPR may advantageously be a fungal demethylase-CPR. In particular, the fungal demethylase-CPR may comprise a polypeptide selected from the group consisting of:
a) a demethylase-CPR which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the demethylase-CPR comprised in SEQ ID NO: 305;
b) a demethylase-CPR encoded by a polynucleotide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the polynucleotide comprised in SEQ ID NO: 306 or genomic DNA thereof; and
c) a functional variant of the demethylase-CPR of (a) or (b) capable of reducing/regenerating the demethylase.

Further suitable demethylases are disclosed in WO2018/229306 and WO2018/075670, which are hereby incorporated by reference in their entirety.

In one embodiment the heterologous demethylase is an artificial mutant. In one type of mutations the naturally occurring leader/signal sequence has been mutated to improve the performance eg. By wholly or partially replacing the the leader/signal sequence with a leader/signal sequence from another enzyme. Examples of such mutations are SEQ ID NOS: 845, 847, 851, 853, 857, 859, 863, 865, 867 and 869.

In another embodiment the demethylase is a polypeptide having at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the demethylase comprised in SEQ ID No. 152.

In another embodiment the demethylase is polypeptide having at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the demethylase comprised in SEQ ID No. 140.

Further the invention provides mutant insect demethylases comprising one or more mutations in the signal sequence of the naturally occurring insect demethylase. In these insect demethylases the signal sequence may have been wholly or partially replaced by a signal sequence from another enzyme. Suitably such a mutant demethylase is a polypeptide having having at least 70%, such as at least 80%, such as at least 90%, such as at least 95%, such as at least 96%, such as at least 97%, such as at least 98%, such as at least 99%, such as 100% identity to the demethylase comprised in SEQ ID NO: 845, 847, 851, 853, 857, 859, 863, 865, 867 or 869. Also mutant insect demethylases are provided having least 70%, such as at least 80%, such as at least 90%, such as at least 95%, such as at least 96%, such as at least 97%, such as at least 98%, such as at least 99%, such as 100% identity to the demethylase comprised in SEQ ID NO: 152. Still further, mutant insect demethylases are provided having at least 70%, such as at least 80%, such as at least 90%, such as at least 95%, such as at least 96%, such as at least 97%, such as at least 98%, such as at least 99%, such as 100% identity to the demethylase comprised in SEQ ID NO: 140.

Analysis comparing the best performing insect demethylases was shown share structural sequence features in the form of amino acid positions conserved within the active and high performing insect demethylases.

### Heterologous TH—Tyrosine Hydroxylase

In another aspect the host cell of the invention expresses alone or in combination with other heterologous genes of the invention one or more heterologous genes encoding a tyrosine hydroxylases. The TH of the invention may suitably be any natural or mutant TH capable of catalyzing L-tyrosine into L-DOPA. Particularly, the TH is of the CYP76 family. In a special embodiment the TH has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the TH comprised in SEQ ID NO: 7, 9, 11, 13, 15, 17, 19, 21, 23, 25, 27, 29, 31, 33, 35, 37, 39, 41, 43, 45, 47, 49, 51, 53, 55, 57, 59, 61, 63 or 65. In a separate embodiment the TH has at least 90%, such as at least 95%, such as at least 96%, such as at least 97%, such as at least 98%, such as at least 99%, such as 100% identity to the TH comprised in SEQ ID NO: 7, 9, 11, 13, 15, 17, 19, 21, 23 or 25. Further suitable THs are disclosed in PCT/EP2020/050610 (unpublished) and WO2016/049364, which are hereby incorporated by reference in its entirety.

### Reducing or Eliminating Enzymes Lowering Performance of the Benzylisoquinoline Alkaloid Pathway

In another aspect the host cell of the invention is genetically modified to reduce or eliminate (knockout) activity of one or more native enzymes, which negatively impacts on the production of benzylisoquinoline alkaloid. Such manipulation may be achieved in several ways, all applicable to the host cell of the invention. Reduction or elimination of enzyme activity may be accomplished by disrupting, deleting and/or attenuating expression of the gene encoding the enzyme and/or the translation of the RNA into the protein, eg. by deleting or mutating the gene. Alternatively, and/or in addition, the the enzyme may also be mutated to a less active or non-active variant. In reducing or eliminating activity activity of enzymes native to the host care should be taken to balance the positive impact on production of benzylisoquinoline alkaloid and the potential negative impact on cellular viability and growth for maintain an acceptable level of vital cellular functions.

Reduction or elimination of activity of enzymes native to the host cell, particularly includes reduction or elimination enzymes shunting precursors or products away from the benzylisoquinoline alkaloid pathway, so that they become unavailable for producing benzylisoquinoline alkaloids. One such group of such enzymes is dehydrogenases native to the host cell and in particular dehydrogenases comprised in SEQ ID NO: 663, 665, 667, 669, 671, 673, 675, 677, 679, 681, 683, 685, 687, 689, 691, 693, 695, 697, 699, 701, 703 or 705. Another group of such enzymes are reductases native to the host cell and in particular reductases comprised in SEQ ID NO: 707, 709, 711, 713, 715, 717, 719, 721, 723, 725, 727, 729 or 731. Preferred targets of reduction or elimination are one or more enzymes comprised in SEQ ID NO: 665 (ADH6), 669 (YPR1), 671 (AAD3), 675 (ADH3), 679 (ALD6), 705 (HFD1), 709 (ALD4), 713 (GRE2), 717 (YDR541C), 721 (ARI1), 729 (PHA2) or 731 (TRP3). Reduction or elimination of one or more the enzymes comprised in 705 (HFD1), 713 (GRE2) or 721 (ARI1), is particularly useful.

Further useful knockouts are disclosed in WO2019/243624, WO2018/029282, WO2019/157383 and Pyne et al, BioRxiv preprint 2019; all hereby incorporated by reference in their entirety.

### Heterologous Norcoclaurine Synthase (NCS)

In a further aspect the host cell of the invention expresses alone or in combination with other heterologous genes of the invention one or more heterologous gene encoding a norcoclaurine synthase (NCS). The NCS of the invention may suitably be any natural or mutant NCS capable of converting Dopamine and 4-HPAA into (S)-norcoclaurine. In a special embodiment the NCS has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the NCS comprised in SEQ ID NO: 73 OR 76. In a separate embodiment the NCS has at least 90%, such as at least 95%, such as at least 96%, such as at least 97%, such as at least 98%, such as at least 99%, such as 100% identity to the NCS comprised in SEQ ID NO: 73 OR 76. Further suitable NCSs are disclosed in WO2018/229305, WO2014/143744, WO2019/165551 and US2015267233, which is hereby incorporated by reference in its entirety.

### Heterologous STORR

In a further aspect the host cell of the invention expresses alone or in combination with other heterologous genes of the invention one or more heterologous genes encoding enzymes capable of epimerizing/isomerizing one benzylisoquinoline alkaloid to a benzylisoquinoline alkaloid isomer, such as for example(S)-Reticuline into (R)-reticuline. In a special embodiment the epimerase is:
i) a fused 1,2-dehydroreticuline synthase-1,2-dehydroreticuline reductases (DRS-DRR) converting (S)-Reticuline into (R)-reticuline, wherein:
   a) the DRS-DDRs has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DRS-DRR comprised in SEQ ID NO: 92, 94, 96; or
   b) the DRS moiety has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DRS comprised in SEQ ID NO: 98, 100, 102, 104 or 106; and the DRR moiety has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DRR comprised in SEQ ID NO: 108 or 110; or
ii) a DRS having at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DRS comprised in SEQ ID NO: 98, 100, 102, 104 or 106; and a DRR having at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DRR comprised in SEQ ID NO: 108 or 110.

In a particular embodiment the DRR moiety of the epimerase, whether fused to the DRS or separate an Imine reductase, preferably a StlRED such as the reductases comprised in SEQ ID NO. 108 or 110.

Further suitable epimerases/isomerases are disclosed in WO2015/081437, WO2016/183023, WO2015/173590, WO2018/000089, WO2019/028390 and WO2019/165551 which are hereby incorporated by reference in their entirety.

### Heterologous THS

In another aspect the host cell of the invention expresses alone or in combination with other heterologous genes of the invention one or more heterologous genes encoding a thebaine synthase (THS). The THS of the invention may suitably be any natural or mutant THS capable of converting 7-O-acetylsalutaridinol into thebaine. In a special embodiment the THS has is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the THS comprised in SEQ ID NO: 126, 127, 128, 129, 131, 133, 134, 136 or 138. In particular SEQ ID NO: 134 and 136 are very efficient thebaine synthases.

Further suitable THSs are disclosed in WO2018/005553, WO2014/143744 and WO2019/165551, which are hereby incorporated by reference in their entirety.

### Heterologous Uptake Transporters

In another aspect the host cell of the invention expresses alone or in combination with other heterologous genes of the invention one or more heterologous genes encoding an uptake transporter protein. The heterologous uptake transporter protein may suitably be any natural or mutant transporter protein capable of net uptake (i.e. influx) of a BIA or BIA intermediate, such as a reticuline derivative, such as thebaine or oripavine. Preferably, the uptake transporter is selected based on increased specificity of a substrate fed into or intermediate in the BIA-producing biosynthetic pathway of the recombinant microbial host cell compared to the one or more BIA, BIA-glycoside, oripavine, glucosylated oripavine, gly-oripavine, thebaine, northebaine, nororipavine, gly-nororipavine, glucosylated nororipavine, nor-opioids or glycosylated noropioids produced by the recombinant microbial host cell. In some aspects, the heterologous uptake transporter may be a purine permease (PUP transporter).

In a special embodiment the uptake transporter protein has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the transporter protein comprised in SEQ ID NO: 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 733, 735, 778, 780, 782, 784, 786, 788, 790, 792, 794, 795, 797, 799, 801, 803, 805, 807, 809, 811, 813, 815, 817, 819, 821, 823 or 825.

Selecting the optimal uptake transporter for a given bioconversion or recombinant pathway setup may depend on the substrate used for bioconversion. So, in a particular embodiment when incorporating a demethylase, especially an insect demethylase, converting oripavine into nororipavine, oripavine uptake transporters are preferred. In particular transporters T180_McoPUP3_46 (SEQ ID NO: 595), T193_AanPUP3_55 (SEQ ID NO: 613), T149_AcoPUP3_59 (SEQ ID NO: 537) and/or T165_AcoPUP3_13 (SEQ ID NO: 567) have shown particularly effective. In another embodiment when in incorporating a demethylase, especially an insect demethylase, converting thebaine into northebaine, thebaine uptake transporters are preferred. In particular transporters T193_AanPUP3_55 (SEQ ID NO: 613), T198_AcoT97_GA (SEQ ID NO: 623), T149_AcoPUP3_59 (SEQ ID NO: 537) and/or T122_PsoPUP3_17 (SEQ ID NO: 487) have shown particularly effective. Further suitable transporter proteins are disclosed in WO2020/078837, which is hereby incorporated by reference in its entirety.

In a further separate embodiment, the transporter may be an Equilibrative Nucleoside Transporter (ENT) as described in Boswell-Casteel and Hays, 2017. Equilibrative Nucleoside Transporters including those belonging to the SLC29A/ENT transporter (TC 2.A.57) family (https://www.uniprot.org) have been shown herein to be capable of demethylase-mediated bioconversion of methylated benzylisoquinoline alkaloids to the corresponding nor-benzylisoquinoline alkaloids—in particular oripavine to nororipavine—in a highly efficient manner. Such improvements in yield are particularly remarkable and represent a significant step forward towards a sustainable, secure, and scalable biosynthetic means of producing these compounds.

The Equilibrative Nucleoside Transporter may particularly be an insect Equilibrative Nucleoside Transporter, including the transporters having at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the transporter protein comprised in SEQ ID NOS: 795, 797, 799, 801, 803, 805, 807, 809, 811, 813, 815, 817, 819, 821, 823 or 825, especially SEQ ID NOS: 795, 797, 799, 801.

The useful insect transporters disclosed herein have not hitherto been demonstrated to benefit production of benzylisoquinoline alkaloids when incorporated heterologously in genetically modified microorganisms comprising pathways producing benzylisoquinoline alkaloids. Accordingly, in a separate aspect the invention provides a genetically modified host cell comprising a pathway having enhanced production of one or more benzylisoquinoline alkaloids wherein the cell expresses one or more heterologous genes encoding an insect derived transporter protein increasing the cellular uptake or secretion of a benzylisoquinoline alkaloid precursor, said precursor preferably being a benzylisoquinoline alkaloid itself. Particular insect transporters include transporter proteins from the insect genera of Helicoverpa, Heliothis or Pectinophora, in particular from species of Pectinophora gossypiella, Helicoverpa armigera or Heliothis virescens. In particular the transporter proteins have at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the transporter protein comprised in SEQ ID NO: 631, 633, 637, 649, 651, 653, 655, 657 or 659. Moreover, the genetically modified cell of the invention may comprise one or more copies of genes encoding one or more insect transporter proteins such as genes/polynucleotides which is at least 70% identical to the transporter encoding polynucleotide comprised in SEQ ID NO: 632, 634, 638, 652, 654, 656, 658 or 660 or genomic DNA thereof.

### Further Enzymes of the Benzylisoquinoline Alkaloid Pathway

In another aspect the host cell of the invention expresses in combination with other heterologous genes of the invention one or more further heterologous or native enzymes of the benzylisoquinoline alkaloid pathway. In a particular embodiment the host cell of the invention expresses one or more genes encoding polypeptides selected from:
a) a 3-deoxy-D-arabino-2-heptulosonic acid 7-phosphate synthase (DAHP synthase) converting PEP and E4P into DAHP;
b) a 5-enolpyruvylshikimate-3-phosphate (EPSP) synthase (aro1) converting 3-phosphoshikimate and PEP into EPSP;
c) an aro1 polypeptide converting DHAP and PEP into EPSP;
d) a chorismate synthase converting EPSP into Chorismate;
e) a chorismate mutase converting Chorismate into prephenate;
f) a prephenate dehydrogenase (Tyr1) converting prephenate into 4-HPP;
g) an aromatic aminotransferase converting 4-HPP into L-Tyrosine;
h) a TH-CPR capable of reducing TH;
i) a L-dopa decarboxylase (DODC) converting L-dopa into dopamine;
j) a Tyrosine decarboxylase (TYDC) converting L-dopa into dopamine;
k) a hydroxyphenylpyruvate decarboxylase (HPPDC) converting 4-HPP into 4-HPAA;
l) a monoamine oxidase converting dopamine into 3,4-DHPAA;
m) a 6-O-methyltransferase (6-OMT) converting (S)-norcoclaurine into (S)-Coclaurine and/or norlaudanosoline into (S)-3′-Hydroxy-coclaurine;
n) a Coclaurine-N-methyltransferase (CNMT) converting (S)-Coclaurine into (S)—N-Methylcoclaurine and/or (S)-3′-hydroxycoclaurine into (S)-3′-hydroxy-N-methyl-coclaurine;
o) a N-methylcoclaurine hydroxylase (NMCH) converting (S)-Coclaurine into (S)-3′-hydroxycoclaurine and/or (S)—N-Methylcoclaurine into (S)-3′-Hydroxy-N-Methylcoclaurine;
p) a 3′-hydroxy-N-methyl-(S)-coclaurine 4′-O-methyltransferase (4′-OMT) converting (S)-3′-Hydroxy-N-Methylcoclaurine into (S)-Reticuline;
q) a DRS-CPR capable of reducing DRS-DRR;
r) a salutaridine synthase (SAS) converting (R)-reticuline into Salutaridine;
s) a salutaridine reductase (SAR) converting Salutaridine to Salutaridinol; and
t) a salutaridinol 7-O-acetyltransferase (SAT) converting Salutaridinol into 7-O-acetylsalutaridinol.

In a special embodiment the corresponding:
a) DAHP synthase has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DAHP synthase comprised in SEQ ID NO: 1
b) chorismate mutase has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the chorismate synthase comprised in SEQ ID NO: 3;
c) TH-CPR has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the TH-CPR comprised in SEQ ID NO: 67;
d) DODC has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DODC comprised in SEQ ID NO: 69 or 71;
e) 6-OMT has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the 6-OMT comprised in SEQ ID NO: 79 or 81;
f) CNMT has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the CNMT comprised in SEQ ID NO: 82 or 84;
g) NMCH has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the NMCH comprised in EQ ID NO: 85 OR 87;
h) 4′-OMT has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the 4′-OMT comprised in SEQ ID NO: 89 or 91;
i) DRS-CPR has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DRS-CPR comprised in SEQ ID NO: 112 or 114;
j) SAS has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the SAS comprised in SEQ ID NO: 116 or 118;
k) SAR has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the SAR comprised in SEQ ID NO: 120 or 122;
l) SAT has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the SAT comprised in SEQ ID NO: 123 or 125; and

Further suitable enzymes of the benzylisoquinoline alkaloid pathway are disclosed in US2019100781 and WO2019/165551, which is hereby incorporated by reference in their entirety.

### Additional Cell Modifications Improving Production of Benzylisoquinoline Alkaloids

During the efforts of improving cellular production of recombinant cellular production of benzylisoquinoline alkaloids, several additional useful modifications to cells improving the cellular performance was discovered. In a first aspect it was found that cytosolic heme levels in a production host cell is a significant limiting factor in production of demethylated nor-benzylisoquinoline alkaloids such as nororipavine and/or northebaine and that modifications to the cell increasing the cytosolic heme levels strongly benefits production of such demethylated nor-benzylisoquinoline alkaloids. Accordingly, in one embodiment the host cell is further modified to increase availability of heme in the cell, in particular by modifying expression of one or more heme expression co-factors in the cell.

In one embodiment the heme availability can be increased by overexpressing and/or co-expressing one or more rate-limiting enzymes from the heme pathway, including but not limited to HEM2, HEM3 and/or HEM12. Overexpression of such genes can be accomplished for example by increasing the number of copies of integrated genes and/or by using stronger promoters of other factors increase translation or transcription of the gene. Preferably both an increase in copy number and use of an appropriate combination of stronger and weaker promoters are used to increase availability of heme. Useful promoters for these gene include pPYK1, pSED1, pKEX2, pTEF1, pTDH3 and pPGK1, where pTEF1, pTDH3 and pPGK1 are the stronger ones. In another embodiment heme availability is increased by disrupting, deleting and/or attenuating any heme-down regulating genes, such as HMX1. In another embodiment heme availability is increased by adding a heme production booster agent such as hemin (Protchenko et al., 2003 and Krainer et al., 2015, respectively).

In a further aspect it was found that overexpressing and/or co-expressing P450 helper genes in a production host cell significantly benefits production of demethylated nor-benzylisoquinoline alkaloids. Such P450 helper genes includes, but is not limited to:
a) DAP1, which encodes a heme-binding protein involved in the regulation the function of cytochrome P450 (Hughes et al., 2007);
b) HAC1, a transcription factor that modulates the unfolded protein response (Kawahara T, et al., 1997);
c) KAR2, HSP82, CNE1, SSA1, CPR6, FES1, HSP104 and STI1 involved in protein processing as well as heat shock response (Yu et al., 2017).

In a still further aspect, it was found that increasing cytosolic levels of NADPH by overexpressing and/or co-expressing genes in the pentose metabolic pathway significantly benefits production of demethylated nor-benzylisoquinoline alkaloids. Such genes include but is not limited to ZWF1 and GND1 genes from the pentose phosphate pathway (Stincone et al., 2015).

In a further aspect it was found that detoxifying the genetically modified cell from formaldehyde, a toxic by-product released during cytochrome P450 N-demethylation reaction (Wehner E P et al., 1993 and Kalász H et al., 1998), significantly benefits production of demethylated nor-benzylisoquinoline alkaloids. Lowering formation of cytosolic formaldehyde in the cell can be achieved modifying genes encoding factors regulating formaldehyde levels and/or toxicity. Such genes/factors include but is not limited to SFA1, which when overexpressed and/or co-expressed reduce formaldehyde levels and/or toxicity and thereby increase production of demethylated nor-benzylisoquinoline alkaloids.

### Functional Homologs

Functional homologs (also referred herein to as functional variants) of the enzymes/polypeptides described herein are also suitable for use in producing benzylisoquinoline alkaloid in the genetically modified host cell. A functional homolog is a polypeptide that has sequence similarity to a reference polypeptide, and that carries out one or more of the biochemical or physiological function(s) of the reference polypeptide. A functional homolog and the reference polypeptide can be a natural occurring polypeptide, and the sequence similarity can be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, can themselves be functional homologs. Functional homologs can also be created via site-directed mutagenesis of the coding sequence for a polypeptide, or by combining domains from the coding sequences for different naturally-occurring polypeptides (“domain swapping”). Techniques for modifying genes encoding functional polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide-polypeptide interactions in a desired manner. Such modified polypeptides are considered functional homologs. The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of benzylisoquinoline alkaloid biosynthesis polypeptides. Sequence analysis can involve BLAST, Reciprocal BLAST, or PSI-BLAST analysis of non-redundant databases using a UGT amino acid sequence as the reference sequence. Amino acid sequence is, in some instances, deduced from the nucleotide sequence. Those polypeptides in the database that have greater than 40% sequence identity are candidates for further evaluation for suitability as a benzylisoquinoline alkaloid biosynthesis polypeptide. Amino acid sequence similarity allows for conservative amino acid substitutions, such as substitution of one hydrophobic residue for another or substitution of one polar residue for another. If desired, manual inspection of such candidates can be carried out to narrow the number of candidates to be further evaluated. Manual inspection can be performed by selecting those candidates that appear to have domains present in benzylisoquinoline alkaloid biosynthesis polypeptides, e.g., conserved functional domains. In some embodiments, nucleic acids and polypeptides are identified from transcriptome data based on expression levels rather than by using BLAST analysis. Methods for conservative substitution are known to the skilled person, see for example https://www.ncbi.nlm.nih.gov/pmc/articles/PMC1449787/or https://link.springer.com/article/10.1007/BF02300754

Conserved regions can be identified by locating a region within the primary amino acid sequence of a benzylisoquinoline alkaloid biosynthesis polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on for example the World Wide Web at sanger.ac.uk/Software/Pfam/and pfam.janelia.org/. The information included at the Pfam database is described in Sonnhammer et al. (1998); Sonnhammer et al. (1997); and Bateman et al. (1999). Conserved regions also can be determined by aligning sequences of the same or related polypeptides from closely related species. Closely related species preferably are from the same family. In some embodiments, alignment of sequences from two different species is adequate to identify such homologs.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity.

For example, polypeptides suitable for producing one or more benzylisoquinoline alkaloids (any BIA, BIA-glycoside, oripavine, glucosylated oripavine, gly-oripavine, thebaine, northebaine, nororipavine, gly-nororipavine, glucosylated nororipavine, nor-opioids or glycosylated noropioids) in a genetically modified host cell include functional homologs of TH's, NCS's, 6-OMT's, CNMT's, NMCH's, 4′-OMT's, DRS-DRR's, SAS's, SAR's, SAT's, THS's, CPR's and demethylating P450's.

Methods to modify the substrate specificity of benzylisoquinoline alkaloids pathway enzymes are known to those skilled in the art, and include without limitation site-directed/rational mutagenesis approaches, random directed evolution approaches and combinations in which random mutagenesis/saturation techniques are performed near the active site of the enzyme. For example see Osmani et al. (2009).

A candidate sequence typically has a length that is from 80% to 200% of the length of the reference sequence, e.g., 82, 85, 87, 89, 90, 93, 95, 97, 99, 100, 105, 110, 115, 120, 130, 140, 150, 160, 170, 180, 190, or 200% of the length of the reference sequence. A functional homolog polypeptide typically has a length that is from 95% to 105% of the length of the reference sequence, e.g., 90, 93, 95, 97, 99, 100, 105, 110, 115, or 120% of the length of the reference sequence, or any range between.

It will be appreciated that functional benzylisoquinoline alkaloids pathway enzymes/polypeptides can include additional amino acids that are not involved in the enzymatic activities carried out by the enzymes. In some embodiments, such enzymes are fusion proteins. The terms “chimera,” “fusion polypeptide,” “fusion protein,” “fusion enzyme,” “fusion construct,” “chimeric protein,” “chimeric polypeptide,” “chimeric construct,” and “chimeric enzyme” can be used interchangeably herein to refer to proteins engineered through the joining of two or more genes that code for different proteins. In some embodiments, a nucleic acid sequence encoding a benzylisoquinoline alkaloids pathway enzyme/polypeptide can include a tag sequence that encodes a “tag” designed to facilitate subsequent manipulation (e.g., to facilitate purification or detection), secretion, or localization of the encoded enzyme. Tag sequences can be inserted in the nucleic acid sequence encoding the polypeptide such that the encoded tag is located at either the carboxyl or amino terminus of the polypeptide. Non-limiting examples of encoded tags include green fluorescent protein (GFP), human influenza hemagglutinin (HA), glutathione S transferase (GST), polyhistidine-tag (HIS tag), and Flag™ tag (Kodak, New Haven, CT). Other examples of tags include a chloroplast transit peptide, a mitochondrial transit peptide, an amyloplast peptide, signal peptide, or a secretion tag.

In some embodiments, a fusion protein is a protein altered by domain swapping. As used herein, the term “domain swapping” is used to describe the process of replacing a domain of a first protein with a domain of a second protein. In some embodiments, the domain of the first protein and the domain of the second protein are functionally identical or functionally similar. In some embodiments, the structure and/or sequence of the domain of the second protein differs from the structure and/or sequence of the domain of the first protein. In some embodiments, a benzylisoquinoline alkaloids pathway enzyme/polypeptide is altered by domain swapping.

### Nucleotides Expressed by the Host Cell

In some aspects, the recombinant microbial host cell comprises a recombinant polynucleotide comprising a promoter operably linked to an ABC transporter, wherein the ABC transporter is a member of the ABCG/pleiotropic drug resistance (PDR) subfamily of ABC transporters or the ABCC/multi-drug resistance associated protein (MRP) subfamily of ABC transporters, and wherein the ABC transporter is capable of effluxing from the host cell one or more opioids or benzylisoquinoline alkaloids selected from a BIA, BIA-glycoside, oripavine, glucosylated oripavine, gly-oripavine, thebaine, northebaine, nororipavine, gly-nororipavine, glucosylated nororipavine, nor-opioids or glycosylated noropioids.

In some aspects the host cell of the invention capable of producing one or more one or more BIA (benzylisoquinoline alkaloids including but not limited to any BIA, BIA-glycoside, oripavine, glucosylated oripavine, gly-oripavine, thebaine, northebaine, nororipavine, gly-nororipavine, glucosylated nororipavine, nor-opioids or glycosylated noropioids) one or more additional polynucleotides or genes selected from:
a) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the DAHP synthase encoding polynucleotide comprised in SEQ ID NO: 2 or genomic DNA thereof;
b) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the chorismate mutase encoding polynucleotide comprised in SEQ ID NO: 4 or genomic DNA thereof;
c) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the TH encoding polynucleotide comprised in SEQ ID NO: 8, 10, 12, 14, 16, 18, 20, 22, 24, 26, 28, 30, 32, 34, 36, 38, 40, 42, 44, 46, 48, 50, 52, 54, 56, 58, 60, 62, 64 or 66 or genomic DNA thereof;
d) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the TH-CPR encoding polynucleotide comprised in SEQ ID NO: 68 or genomic DNA thereof;
e) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the DODC encoding polynucleotide comprised in SEQ ID NO: 70 or 72 or genomic DNA thereof;
f) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the NCS encoding polynucleotide comprised in SEQ ID NO: 74 or 77 or genomic DNA thereof;
g) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the 6-OMT encoding polynucleotide comprised in SEQ ID NO: 80 or genomic DNA thereof;
h) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the CNMT encoding polynucleotide comprised in SEQ ID NO: 83 or genomic DNA thereof;
i) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the NMCH encoding polynucleotide comprised in SEQ ID NO: 86 or 88 or genomic DNA thereof;
j) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the 4′-OMT encoding polynucleotide comprised in SEQ ID NO: 90 or genomic DNA thereof;
k) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the DRS-DRR encoding polynucleotide comprised in SEQ ID NO: 93, 95 or 97 or genomic DNA thereof;
l) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the DRS encoding polynucleotide comprised in SEQ ID NO: 99, 101, 103, 105 or 107 or genomic DNA thereof;
m) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the DRR encoding polynucleotide comprised in SEQ ID NO: 109 or 111 or genomic DNA thereof;
n) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the DRS-CPR encoding polynucleotide comprised in SEQ ID NO: 113 or 115 or genomic DNA thereof;
o) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the SAS encoding polynucleotide comprised in SEQ ID NO: 117 or 119 or genomic DNA thereof;
p) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the SAR encoding polynucleotide comprised in SEQ ID NO: 121 or genomic DNA thereof;
q) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the SAT encoding polynucleotide comprised in SEQ ID NO: 124 or genomic DNA thereof;
r) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the THS encoding polynucleotide comprised in SEQ ID NO: 130, 132, 135, 137 or 139 or genomic DNA thereof;
s) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the ODM encoding polynucleotide comprised in SEQ ID NO: 219, 221, 223, 225, 227, 229, 237, 241, 251, 253, 255 and 267 or genomic DNA thereof;
t) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the demethylase encoding polynucleotide comprised in any one of SEQ ID NO: 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 828, 830, 832, 834, 836, 838, 840, 842, 844, 846, 848, 850, 852, 854, 856, 858, 860, 862, 864, 866, 868 and 870 or genomic DNA thereof;
u) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the demethylase-CPR encoding polynucleotide comprised in any one of SEQ ID NO: 293, 295, 297, 299, 301, 303, 304 or 306 or genomic DNA thereof; and
v) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the transporter encoding polynucleotide comprised in SEQ ID NO: 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 734, 736, 777, 779, 781, 783, 785, 787, 789, 781, 783, 785, 787, 789, 791, 793, 796, 798, 800, 801, 802, 804, 806, 808, 810, 812, 814, 816, 818, 820, 822, 824, 826 or genomic DNA thereof.

Any nucleotides disclosed herein may be codon optimized for expression in a particular selected host using methods available to the skilled person or commercially available from technology providers-see for example Gene Reports Volume 9, December 2017, Pages 46-53: Strategies of codon optimization for high-level heterologous protein expression in microbial expression systems, incorporated herein by reference. Examples of codon optimized genes are those of SEQ ID NOS: 771, 773, 775, 777, 779, 781, 783, 785, 787, 789, 791 and 793.

### Host Cells.

The cell of the invention may be any host cell suitable for hosting and expressing the BIA efflux transporters of the invention and capable of one or more benzylisoquinoline alkaloids (including but not limited to any BIA, BIA-glycoside, oripavine, glucosylated oripavine, gly-oripavine, thebaine, northebaine, nororipavine, gly-nororipavine, glucosylated nororipavine, nor-opioids or glycosylated noropioids).

In particular the cell of the invention may be a eukaryote cell selected from the group consisting of mammalian, insect, plant, or fungal cells In another embodiment the cell is a fungal cell selected from the phylas consisting of Ascomycota, Basidiomycota, Neocallimastigomycota, Glomeromycota, Blastocladiomycota, Chytridiomycota, Zygomycota, Oomycota and Microsporidia. A particularly useful fungal cell is a yeast cell selected from the group consisting of ascosporogenous yeast (Endomycetales), basidiosporogenous yeast, and Fungi Imperfecti yeast (Blastomycetes). Such yeast cells may further be selected from the genera consisting of Saccharomyces, Kluveromyces, Candida, Pichia, Debaromyces, Hansenula, Yarrowia, Zygosaccharomyces, and Schizosaccharomyces. More specifically the yeast cell may be selected from the species consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica.

An alternative fungal host cell of the invention is a filamentous fungal cell. Such filamentous fungal cell may be selected from the phylas consisting of Ascomycota, Eumycota and Oomycota, more specifically selected from the genera consisting of Acremonium, Aspergillus, Aureobasidium, Bjerkandera, Ceriporiopsis, Chrysosporium, Coprinus, Corio/us, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma. In important embodiments the filamentous fungal cell may be selected from the species consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporiuminops, Chrysosporiumkeratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride.

In one embodiment the cell is a plant cell for example of the genus Physcomitrella or Papaver, in particular Papaver somniferum. Other plant cells can be of the family Solanaceae, such genuses of Nicotiana, such as Nicotiana benthamiana. In addition to plant cells the invention also provides an isolated plant, e.g., a transgenic plant, plant part comprising the benzylisoquinoline alkaloid pathway polypeptides of the invention and producing the benzylisoquinoline alkaloids of the invention in useful quantities. The compound may be recovered from the plant or plant part. The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn). Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana. Examples of plant parts are stem, callus, leaves, root, fruits, seeds, and tubers as well as the individual tissues comprising these parts, e.g., epidermis, mesophyll, parenchyme, vascular tissues, meristems. Specific plant cell compartments, such as chloroplasts, apoplasts, mitochondria, vacuoles, peroxisomes and cytoplasm are also considered to be a plant part. Furthermore, any plant cell, whatever the tissue origin, is considered to be a plant part. Likewise, plant parts such as specific tissues and cells isolated to facilitate the utilization of the invention are also considered plant parts, e.g., embryos, endosperms, aleurone and seed coats. Also included within the scope of the present invention is any the progeny of such plants, plant parts, and plant cells. The transgenic plant or plant cells comprising the operative pathway of the invention and produce the compound of the invention may be constructed in accordance with methods known in the art. In short, the plant or plant cell is constructed by incorporating one or more expression vectors of the invention into the plant host genome or chloroplast genome and propagating the resulting modified plant or plant cell into a transgenic plant or plant cell. The expression vector conveniently comprises the polynucleotide construct of the invention. The choice of regulatory sequences, such as promoter and terminator sequences and optionally signal or transit sequences, is determined, for example, on the basis of when, where, and how the pathway polypeptides is desired to be expressed. For instance, the expression of a gene encoding a pathway enzyme polypeptide may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86:506. For constitutive expression, the 358-CaMV, the maize ubiquitin 1, or the rice actin 1 promoter may be used (Franck et al., 1980, Cell 21:285-294; Christensen et al., 1992, Plant Mol. Biol. 18:675-689; Zhang et al., 1991, Plant Cell 3:1155-1165). Organ-specific promoters may be, for example, a promoter from storage sink tissues such as seeds, potato tubers, and fruits (Edwards and Coruzzi, 1990, Ann. Rev. Genet. 24:275-303), or from metabolic sink tissues such as meristems (Ito et al., 1994, Plant Mol. Biol. 24:863-878), a seed specific promoter such as the glutelin, prolamin, globulin, or albumin promoter from rice (Wu et al., 1998, Plant Cell Physiol. 39:885-889), a Vicia faba promoter from the legumin B4 and the unknown seed protein gene from Vicia faba (Conrad et al., 1998, J. Plant Physiol. 152:708-711), a promoter from a seed oil body protein (Chen et al., 1998, Plant Cell Physiol. 39:935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiol. 102:991-1000), the chlorella virus adenine methyltransferase gene promoter (Mitra and Higgins, 1994, Plant Mol. Biol. 26:85-93), the aldP gene promoter from rice (Kagaya et al., 1995, Mol. Gen. Genet. 248:668-674), or a wound inducible promoter such as the potato pin2 promoter (Xu et al., 1993, Plant Mol. Biol. 22:573-588). Likewise, the promoter may be induced by abiotic treatments such as temperature, drought, or alterations in salinity or induced by exogenously applied substances that activate the promoter, e.g., ethanol, oestrogens, plant hormones such as ethylene, abscisic acid, and gibberellic acid, and heavy metals. A promoter enhancer element may also be used to achieve higher expression in the plant. For instance, the promoter enhancer element may be an intron that is placed between the promoter and the polynucleotide encoding a polypeptide or domain. For instance, Xu et al., 1993, supra, disclose the use of the first intron of the rice actin 1 gene to enhance expression. The selectable marker gene and any other parts of the expression construct may be chosen from those available in the art. The polynucleotide construct or expression vector is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244:1293; Potrykus, 1990, Bio/Technology 8:535; Shimamoto et al., 1989, Nature 338:274). Agrobacterium tumefaciens-mediated gene transfer is a method for generating transgenic dicots (for a review, see Hooykas and Schilperoort, 1992, Plant Mol. Biol. 19:15-38) and for transforming monocots, although other transformation methods may be used for these plants. A method for generating transgenic monocots is particle bombardment (microscopic gold or tungsten particles coated with the transforming DNA) of embryonic calli or developing embryos (Christou, 1992, Plant J. 2:275-281; Shimamoto, 1994, Curr. Opin. Biotechnol. 5:158-162; Vasil et al., 1992, Bio/Technology 10:667-674). An alternative method for transformation of monocots is based on protoplast transformation as described by Omirulleh et al., 1993, Plant Mol. Biol. 21:415-428. Additional transformation methods include those described in U.S. U.S. Pat. Nos. 6,395,966 and 7,151,204 (both incorporated herein by reference in their entirety). Following transformation, the transformants having incorporated the expression vector or polynucleotide construct of the invention are selected and regenerated into whole plants according to methods well known in the art. Often the transformation procedure is designed for the selective elimination of selection genes either during regeneration or in the following generations by using, for example, co-transformation with two separate T-DNA constructs or site specific excision of the selection gene by a specific recombinase. In addition to direct transformation of a particular plant genotype with a polynucleotide construct of the invention, transgenic plants may be made by crossing a plant comprising the construct to a second plant lacking the construct. For example, a polynucleotide construct encoding a glycosyl transferase of the invention can be introduced into a particular plant variety by crossing, without the need for ever directly transforming a plant of that given variety. Therefore, the invention encompasses not only a plant directly regenerated from cells which have been transformed in accordance with the invention, but also the progeny of such plants. As used herein, progeny may refer to the offspring of any generation of a parent plant prepared in accordance with the present invention. Such progeny may include a polynucleotide construct of the invention. Crossing results in the introduction of a transgene into a plant line by cross pollinating a starting line with a donor plant line. Non-limiting examples of such steps are described in U.S. Pat. No. 7,151,204. Plants may be generated through a process of backcross conversion. For example, plants include plants referred to as a backcross converted genotype, line, inbred, or hybrid. Genetic markers may be used to assist in the introgression of one or more transgenes of the invention from one genetic background into another. Marker assisted selection offers advantages relative to conventional breeding in that it can be used to avoid errors caused by phenotypic variations. Further, genetic markers may provide data regarding the relative degree of elite germplasm in the individual progeny of a particular cross. For example, when a plant with a desired trait which otherwise has a non-agronomically desirable genetic background is crossed to an elite parent, genetic markers may be used to select progeny which not only possess the trait of interest, but also have a relatively large proportion of the desired germplasm. In this way, the number of generations required to introgress one or more traits into a particular genetic background is minimized.

The host microbial cells of the invention comprising a BIA efflux transporter and capable of producing one or more benzylisoquinoline alkaloid (including but not limited to any BIA, BIA-glycoside, oripavine, glucosylated oripavine, gly-oripavine, thebaine, northebaine, nororipavine, gly-nororipavine, glucosylated nororipavine, nor-opioids or glycosylated noropioids), may be even further modified by one or more of
a) attenuating, disrupting and/or deleting one or more native or endogenous genes of the cell;
b) inserting two or more copies of polynucleotides encoding the P450s, the demethylase-CPR's and/or one or more of the polypeptides comprised in the operative metabolic pathway;
c) increasing the amount of a substrate for at least one polypeptide of the operative metabolic pathway; and/or
d) increasing tolerance towards one or more substrates, intermediates, or product molecules from the operative metabolic pathway.

### Polynucleotide Constructs and Expression Vectors

In a separate aspect the invention also provides a polynucleotide construct comprising a polynucleotide sequence encoding a heterologous enzymes or transporter protein of the invention operably linked to one or more control sequences, which direct expression of the heterologous enzyme or transporter protein in the host cell harbouring the polynucleotide construct. Conditions for the expression should be compatible with the control sequences. In particular, the control sequence is heterologous to the polynucleotide encoding the heterologous enzyme or transporter protein and in one embodiment the polynucleotide sequence encoding the heterologous enzyme or transporter protein and the control sequence are both heterologous to the host cell comprising the construct. In one embodiment the polynucleotide construct is an expression vector, comprising the polynucleotide sequence encoding the heterologous enzyme or transporter protein of the invention operably linked to the one or more control sequences.

Polynucleotides may be manipulated in a variety of ways allow expression of the heterologous enzyme or transporter protein. Manipulation of the polynucleotide prior to its insertion into an expression vector may be desirable or necessary depending on the expression vector. The techniques for modifying polynucleotides utilizing recombinant DNA methods are well known in the art.

The control sequence may be a promoter, which is a polynucleotide that is recognized by a host cell for expression of a polynucleotide. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell. The promoter may also be an inducible promoter.

Examples of suitable promoters for directing transcription of the nucleic acid construct of the invention in fungal host cell are promoters obtained from the genes for Aspergillus nidulans acetamidase, Aspergillus niger neutral α-amylase, Aspergillus niger acid stable α-amylase, Aspergillus niger or Aspergillus awamori glucoamylase (glaA), Aspergillus gpdA promoter, Aspergillus oryzae TAKA amylase, Aspergillus oryzae alkaline protease, Aspergillus oryzae triose phosphate isomerase, A. niger or A. awamori endoxylanase (xInA) or β-xylosidase (xInD), Fusarium oxysporum trypsin-like protease (WO 96/00787), Fusarium venenatum amyloglucosidase (WO2000/56900), Fusarium venenatum Dania (WO 00/56900), Fusarium venenatum Quinn (WO 00/56900), Rhizomucor miehei lipase, Rhizomucor miehei aspartic proteinase, Trichoderma reesei β-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei β-xylosidase, as well as the NA2-tpi promoter and mutant, truncated, and hybrid promoters thereof. NA2-tpi promoter is a modified promoter from an Aspergillus neutral α-amylase gene in which the untranslated leader has been replaced by an untranslated leader from an Aspergillus triose phosphate isomerase gene. Examples of such promoters include modified promoters from an Aspergillus niger neutral α-amylase gene in which the untranslated leader has been replaced by an untranslated leader from an Aspergillus nidulans or Aspergillus oryzae triose phosphate isomerase gene. Other examples of promoters are the promoters described in WO2006/092396, WO2005/100573 and WO2008/098933, incorporated herein by reference.

Examples of suitable promoters for directing transcription of the nucleic acid construct of the invention in a yeast host include the glyceraldehyde-3-phosphate dehydrogenase promoter, PgpdA or promoters obtained from the genes for Saccharomyces cerevisiae enolase (EN0-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8:423-488. Selecting a suitable promoter for expression in yeast is well know and is well understood by persons skilled in the art.

The control sequence may also be a transcription terminator, which is recognized by a host cell to terminate transcription. The terminator is operably linked to the 3′-terminus of the polynucleotide encoding the polypeptide. Any terminator that is functional in the host cell may be used.

Useful terminators for fungal host cells can be obtained from the genes encoding Aspergillus nidulans anthranilate synthase, Aspergillus niger glucoamylase, Aspergillus niger α-glucosidase, Aspergillus oryzae TAKA amylase, and Fusarium oxysporum trypsin-like protease; while useful terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

The control sequence may also be a leader, a non-translated region of an mRNA that is important for translation by the host cell. The leader is operably linked to the 5′-terminus of the polynucleotide encoding the polypeptide. Any leader that is functional in the host cell may be used.

Useful leaders for fungal host cells can be obtained from the genes for Aspergillus oryzae TAKA amylase and Aspergillus nidulans triose phosphate isomerase, while useful leaders for yeast host cells can be obtained from the genes for Saccharomyces cerevisiae enolase (EN0-1), Saccharomyces cerevisiae 3-phosphoglycerate kinase, Saccharomyces cerevisiae α-factor, and Saccharomyces cerevisiae alcohol dehydrogenase/glyceraldehyde-3-phosphate dehydrogenase (ADH2/GAP).

The control sequence may also be a polyadenylation sequence; a sequence operably linked to the 3′-terminus of the polynucleotide and, when transcribed, is recognized by the host cell as a signal to add polyadenosine residues to transcribed mRNA. Any polyadenylation sequence that is functional in the host cell may be used. Useful polyadenylation sequences for fungal host cells can be obtained from the genes for Aspergillus nidulans anthranilate synthase, Aspergillus niger glucoamylase, Aspergillus niger α-glucosidase Aspergillus oryzae TAKA amylase, and Fusarium oxysporum trypsin-like protease; while useful polyadenylation sequences for yeast host cells are described by Guo and Sherman, 1995, Mol. Cellular Biol. 15:5983-5990.

It may also be desirable to add regulatory sequences that regulate expression of the polypeptide relative to the growth of the host cell. Examples of regulatory systems are those that cause expression of the gene to be turned on or off in response to a chemical or physical stimulus, including the presence of a regulatory compound. In fungi, the Aspergillus niger glucoamylase promoter, Aspergillus oryzae TAKA α-amylase promoter, and Aspergillus oryzae glucoamylase promoter may be used; while in yeast, the ADH2 system or GAL 1 system may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals.

Various nucleotide sequences in addition to the polynucleotide construct of the invention may be joined together to produce a recombinant expression vector, which may include one or more convenient restriction sites to allow for insertion or substitution of the polynucleotide sequence encoding the P450 of the invention at such sites. The recombinant expression vector may be any vector (e.g., a plasmid or virus or chromosomal) that can be conveniently subjected to recombinant DNA procedures and can bring about expression of the P450 encoding polynucleotide. The choice of the vector will typically depend on the compatibility of the vector with the host cell into which the vector is to be introduced. The vector may be an autonomously replicating vector, i.e., a vector that exists as an extrachromosomal entity, the replication of which is independent of chromosomal replication, e.g., a plasmid (linear or closed circular plasmid), an extrachromosomal element, a minichromosome, or an artificial chromosome. The vector may contain any means for assuring self-replication. Alternatively, the vector may, when introduced into the host cell, integrate into the genome and replicate together with the chromosome(s) into which it has been integrated. Furthermore, a single vector or plasmid or two or more vectors or plasmids that together contain the total DNA to be introduced into the genome of the host cell, or a transposon, may be used.

The vector may contain one or more selectable markers that permit easy selection of transformed, transfected, transduced, or the like cells. A selectable marker is a gene from which the product provides for biocide or viral resistance, resistance to heavy metals, prototrophy to auxotrophs, and the like. Useful selectable markers for fungal host cells include amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Useful selectable markers for yeast host cells include, but are not limited to, ADE2, HIS3, LEU2, LYS2, MET3, TRP1, and URA3.

The vector may further contain element(s) that permits integration of the vector into genome of the host cell or permits autonomous replication of the vector in the cell independent of the genome. For integration into the host cell genome, the vector may rely on the polynucleotide encoding the P450 or any other element of the vector for integration into the genome by homologous or non-homologous recombination. Alternatively, the vector may contain additional polynucleotides for directing integration by homologous recombination into the genome of the host cell at precise location(s) in the chromosome(s). To increase the likelihood of integration at a precise location, the integrational elements should contain a sufficient number of nucleic acids, such as 100 to 10,000 base pairs, such as 400 to 10,000 base pairs, and such as 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” refers to a polynucleotide that enables a plasmid or vector to replicate in vivo. Useful origins of replication for fungal cells include AMA 1 and ANS1 (Gems et al., 1991, Gene 98:61-67; Cullen et al., 1987, Nucleic Acids Res. 15:9163-9175; WO 00/24883). Isolation of the AMA 1 sequence and construction of plasmids or vectors comprising the gene can be accomplished using the methods disclosed in WO2000/24883. Useful origins of replication for yeast host cells are the 2-micron origin of replication, ARS1, ARS4, the combination of ARS1 and CEN3, and the combination of ARS4 and CEN6.

As mentioned, supra, more than one copy of a polynucleotide encoding the P450 of the invention may be inserted into a host cell to increase production of the P450. An increase in the copy number can be obtained by integrating one or more additional copies of the enzyme coding sequence into the host cell genome or by including an amplifiable selectable marker gene with the polynucleotide, so that cells containing amplified copies of the selectable marker gene- and thereby additional copies of the polynucleotide—can be selected by cultivating the cells in the presence of the appropriate selectable agent. The procedures used to ligate the elements described above to construct the recombinant expression vectors of the present disclosure are well known to one skilled in the art (see, e.g., Sambrook et al., 1989, supra).

In alignment with the above the vehicles of this disclose also include those comprising a microbial host cell comprising the polynucleotide construct as described, supra.

### Cultures

The invention also provides a cell culture, comprising any recombinant microbial host cell of the invention comprising a BIA-efflux transporter and capable of producing one or more BIA (benzylisoquinoline alkaloids including but not limited to any BIA, BIA-glycoside, oripavine, glucosylated oripavine, gly-oripavine, thebaine, northebaine, nororipavine, gly-nororipavine, glucosylated nororipavine, nor-opioids or glycosylated noropioids), and a growth medium. Suitable growth mediums for host cells such as mammalian, insect, plant, fungal and/or yeast cells are known in the art. Various recognized media are known by those skilled in the art for host cell cultivation. Complex media can be used which contain multi-component ingredients such as yeast extract, peptone, tryptic digests, molasses, and casamino acids. For example, a commonly used complex medium for yeasts is YPD (Sigma Aldrich). Alternatively, media can be defined, or minimal, meaning the exact composition is known. Commonly used defined media for yeasts includes Synthetic Minimal medium, Synthetic Complete medium (Sigma Aldrich), Yeast Nitrogen Base, Yeast Synthetic drop-out medium, DELFT synthetic medium, and Verduyne medium. A defined medium or complex medium may be modified from the standard recipe or supplemented with additional components using routine optimization techniques known to those skilled in the art, depending on the specific strain requirements, length and size of fermentation, and the specific fermentation vessel employed. For example, one or more metals (including trace metals), chelators/complexing agents, nitrogen sources, cofactors and vitamins can be used to supplement fermentation media to improve growth and/or productivity. Yeast extract may be added to defined media in concentrations of, for example 0.1-25 g/L, or 0.5-10 g/L. Sources of divalent cations, such as calcium chloride, may be added at 0.05-5 g/L, or from about 0.1-0.5 g/L. Other divalent cations such as manganese can be added to a final concentration of 2-100 mg/L, or 10-50 mg/L. In some embodiments, ferrous sulfate can be added to final concentrations of 0.5-100 mg/L, or 5-50 mg/L. In some embodiments, 0.01-0.04 mM copper (II) is used. In some embodiments, ZnSO4 heptahdyrate can be used from a concentration of 25-150 mg/L. In some embodiments 7-12 mMol Mg is used. In some embodiments, different sources of monovalent salts are used, such as potassium potassium sulfate and/or potassium phosphate (monobasic or dibasic). In some embodiments, chelators/complexing agents such as EDTA and citrate may be used to bind trace metals, such as 0-200 mg/L EDTA or citrate may be used. In some embodiments, the trace vitamins may be optimized for a particular strain and fermentation, for example biotin, inositol, pantothenate, or pyridoxine. As an example, inositol can be modified to use 0.15-5 mM final concentrations in the medium. One skilled in the art knows that different carbon sources in addition to glucose (dextrose) can be used, depending on the host organism and their catabolic enzymes and transport systems. Some organisms can utilize lactose, glycerol, fructose, sucrose, maltose, pyruvate, succinate, fumarate, malate, or carbohydrates such as cellobiose and starch. Some sources of sugars can be complex, such as molasses. One skilled in the art knows that different nitrogen sources may be used for growth and production. For example, ammonium, amino acids, peptides and proteins, or urea.

### Methods of Producing Compounds of the Invention.

The invention also provides a method for producing one or more opiate or benzylisoquinoline alkaloid (such as any BIA, BIA-glycoside, oripavine, glucosylated oripavine, gly-oripavine, thebaine, northebaine, nororipavine, gly-nororipavine, glucosylated nororipavine, nor-opioids or glycosylated noropioids) and/or a derivative thereof comprising:
a) culturing the cell culture of the invention at conditions allowing the cell to produce the benzylisoquinoline alkaloid; and
b) optionally recovering and/or isolating the benzylisoquinoline alkaloid.

In some aspects, the one or more opiate or benzylisoquinoline alkaloid (such as any BIA, BIA-glycoside, oripavine, glucosylated oripavine, gly-oripavine, thebaine, northebaine, nororipavine, gly-nororipavine, glucosylated nororipavine, nor-opioids or glycosylated noropioids) is recovered and/or isolated from microbial host cells of the current invention. In other aspects, the one or more opiate or benzylisoquinoline alkaloid (such as any BIA, BIA-glycoside, oripavine, glucosylated oripavine, gly-oripavine, thebaine, northebaine, nororipavine, gly-nororipavine, glucosylated nororipavine, nor-opioids or glycosylated noropioids) is recovered and/or isolated from the cell culture medium after culturing the microbial host cells of the current invention. In further aspects, the one or more opiate or benzylisoquinoline alkaloid (such as any BIA, BIA-glycoside, oripavine, glucosylated oripavine, gly-oripavine, thebaine, northebaine, nororipavine, gly-nororipavine, glucosylated nororipavine, nor-opioids or glycosylated noropioids) is recovered and/or isolated from from microbial host cells of the current invention and from the cell culture medium after culturing the microbial host cells of the current invention. The cell culture can be cultivated in a nutrient medium and at conditions suitable for production of the nororipavine glucoside and/or nororipavine of the invention and/or propagating cell count using methods known in the art. For example, the culture may be cultivated by shake flask cultivation, or small-scale or large-scale fermentation (including continuous, batch, fed-batch, feed and draw, or solid-state fermentations) in laboratory or industrial fermentors in a suitable medium and under conditions allowing the host cells to grow and/or propagate, optionally to be recovered and/or isolated.

The cultivation can take place in a suitable nutrient medium comprising carbon and nitrogen sources and inorganic salts, using procedures known in the art. Suitable media are available from commercial suppliers or may be prepared according to published recipes (e.g. from catalogues of the American Type Culture Collection). The selection of the appropriate medium may be based on the choice of host cell and/or based on the regulatory requirements for the host cell. Such media are available in the art. The medium may, if desired, contain additional components favoring the transformed expression hosts over other potentially contaminating microorganisms. Accordingly, in an embodiment a suitable nutrient medium comprises a carbon source (e.g. glucose, maltose, molasses, starch, cellulose, xylan, pectin, lignocellolytic biomass hydrolysate, etc.), a nitrogen source (e.g. ammonium sulphate, ammonium nitrate, ammonium chloride, etc.), an organic nitrogen source (e.g. yeast extract, malt extract, peptone, etc.) and inorganic nutrient sources (e.g. phosphate, magnesium, potassium, zinc, iron, etc.).

The cultivation of the host cell may be performed over a period of from about 0.5 to about 50 days. The cultivation process may be a batch process, continuous or fed-batch process, suitably performed at a temperature in the range of 10-50° C. or 10-40° C., for example, from about 15° C. to about 35° C. and/or at a pH, for example, from about 2 to about 10. Preferred fermentation conditions for yeast and filamentous fungi are a temperature in the range of from about 25° C. to about 55° C. and at a pH of from about 3 to about 9. The appropriate conditions are usually selected based on the choice of host cell. Accordingly, in an embodiment the method of the invention further comprises one or more elements selected from:
a) culturing the cell culture in a nutrient medium;
b) culturing the cell culture under aerobic or anaerobic conditions
c) culturing the cell culture under agitation;
d) culturing the cell culture at a temperature of between 20 to 40° C.;
e) culturing the cell culture at a pH of between 3-9; and
f) culturing the cell culture for between 10 hours to 50 days.

In a special embodiment wherein the host cell of the invention expresses a demethylase converting oripavine to nororipavine in the cell, a demethylase-CPR and a transporter, it has been found that for optimal production of nororipavine at a pH from 3.5 to 6.5, such as from 4.0 to 6.0, such as about 4.5 to 5.5 should be maintained for the culturation/fermentation.

The cell culture of the invention may be recovered and/or isolated using methods known in the art. In some aspects, the the compound(s) may be recovered from the nutrient medium by conventional procedures including, but not limited to, centrifugation, filtration, spray-drying, or lyophilization. In a particular embodiment the method includes a recovery and/or isolation step comprising separating a liquid phase of the cell or cell culture from a precipitate, sediment or solid phase of the cell or cell culture to obtain a supernatant and a solids phase. The supernatant and/or solids phase may comprise the one or more opiate or benzylisoquinoline alkaloid (such as any BIA, BIA-glycoside, oripavine, glucosylated oripavine, gly-oripavine, thebaine, northebaine, nororipavine, gly-nororipavine, glucosylated nororipavine, nor-opioids or glycosylated noropioids). In some aspects, the benzylisoquinoline alkaloid is present in the supernatant and is also present partly associated with the host cells and cell debris and should be washed or extracted from the cells with an appropriate solvent or water prior to supplementing the supernatant and subjecting the liquid phase to one or more steps selected from:
a) contacting the supernatant with one or more adsorbent resins in order to obtain at least a portion of the produced benzylisoquinoline alkaloid, then optionally recovering the benzylisoquinoline alkaloid from the resin in a concentrated solution prior to precipitation or crystallisation of the benzylisoquinoline alkaloid;
b) contacting the supernatant with one or more ion exchange or reversed-phase chromatography columns in order to obtain at least a portion of the benzylisoquinoline alkaloid, then optionally recovering the benzylisoquinoline alkaloid from the resin in a concentrated solution prior to precipitation or crystallisation of the benzylisoquinoline alkaloid;
c) extracting the benzylisoquinoline alkaloid from the supernatant, such as by liquid-liquid extraction into an immisible solvent, then optionally evaporating the solvent to concentrate and precipitate the benzylisoquinoline alkaloid or performing further liquid-liquid extraction to recover and concentrate benzylisoquinoline alkaloid prior to crystallisation or precipitation or in order to directly perform a further chemical reaction on benzylisoquinoline alkaloid; and
d) evaporating the solvent of the supernatant to concentrate or precipitate the benzylisoquinoline alkaloid;
   thereby recovering and/or isolating the benzylisoquinoline alkaloid benzylisoquinoline alkaloid benzylisoquinoline alkaloid. In some embodiments the BIA-glycoside is deglycosylated prior to separation of cell solids from a liquid supernatant. In some embodiments, the deglycosylation is done as part of the purification steps such as those listed in steps (a)-(d) above.

The method of the invention may comprise one or more in vitro steps in the process of producing the one or more opiate or benzylisoquinoline alkaloid (such as any BIA, BIA-glycoside, oripavine, glucosylated oripavine, gly-oripavine, thebaine, northebaine, nororipavine, gly-nororipavine, glucosylated nororipavine, nor-opioids or glycosylated noropioids). It may also comprise one or more in vivo steps performed in another cell, such as a plant cell, for example a cell of Papaver somniferum. For example, thebaine and/or oripavine or precursors thereof may be produced in a plant, such as poppy (Papaver somniferum) and isolated therefrom and then fed to a cell culture of the invention for conversions ion into northebaine and/or nororipavine. Accordingly, in one embodiment the method of the invention further comprises feeding the cell culture with exogenous thebaine, oripavine and/or a precursor thereof, and even further where the exogenous thebaine, oripavine and/or precursor thereof is a plant extract.

Desired end products may be for example buprenorphine, naltrexone, naloxone, nalmefene or nalbuphine. Methods of producing glycosylated BIAs and glycosylated opioids (such as oripavine glycosides and/or nororipavine glycosides), genetically modified host cells producing such glycosides, cultures of those host cells, in addition to methods for cultivating such cultures into fermentation compositions and isolating produced oripavine glycosides and/or nororipavine glycosides therefrom in the formation of compositions comprising oripavine glycosides and/or nororipavine glycosides are all taught in PCT/EP2022/062130, the content of which is incorporated herein.

Currently known methods for producing semisynthetic opioids and BIAs (including oxycodone, hydrocodone, hydromorphone, oxymorphone, naloxone, naltrexone, nalmefene, methylnaltrexone, noroxymorphone, buprenorphine) include production via chemical synthesis from thebaine, oripavine, morphine and codeine, mostly commonly from thebaine or oripavine, all four compounds produced by extraction from the opium poppy (Papaver somniferum). The lack of a commercial supply of nororipavine is in part due to the inability of the opium poppy to produce commercially viable concentrations of nororipavine, which is believed to be due to the lack of a naturally occurring N-demethylase enzyme in the opium poppy. High yielding industrially applicable methods of synthesis of nororipavine have not previously been disclosed and production of commercially relevant quantities of nororipavine have not hitherto been available. Thebaine, oripavine, northebaine and/or in particular nororipavine are attractive for use as a starting material due to their chemical structure and functionality allowing efficient installation of the hydroxy group at C-14 position and/or for performing the Diels-Alder reaction on the methoxydiene moiety to produce the backbone of buprenorphine. Nororipavine produced by fermentation/bioconversion has the additional advantage over thebaine and oripavine that the difficult chemical N-demethylation is already completed further enhancing the utility as a starting material for buprenorphine or “NaIs” synthesis. (Machara et. al. Georg Thieme Verlag Stuttgart. New York-Synthesis 2016, 48, 1803-1813).

Separation methods for opiates and other alkaloids are well-known in the art. See, for example PCT/EP2020/078496, for methods of isolation of deglucosylated glycosylated nor-opiates.

In some aspects of the current invention, the recovered and/or isolated BIA, BIA-glycoside, oripavine or glycosylated oripavine or glucosylated oripavine, thebaine, northebaine, nororipavine, glycosylated nororipavine or glucosylated nororipavine produced by recombinant microbial host cells according to the current invention, is converted chemically and/or biochemically into bis-benzyl nororipavine, nalbuphine, morphine, hydromorphone, codeine, hydrocodone, oxycodone, oxymorphone noroxymorphone, noroxymorphinone, buprenorphine, naloxone, naltrexone, or nalmefene. Such methods of conversion are already familiar to those skilled in the art and applicable regardless of the source of BIA or BIA derivative used, non-limiting examples of which are taught in Carroll et al. 2009, Dasgupta et al. 2020, Fossati et al. 2015, Galanie et al. 2015, Hudlicky et al. 2015, WO2018211331 and WO 2021/144362, which are incorporated herein by reference.

### Fermentation Composition

The invention further provides a fermentation composition comprising the cell culture of the invention and the benzylisoquinoline alkaloid comprised therein.

In one embodiment at least 10%, 25%, 50%, such as at least 75%, such as at least 95%, such as at least 99% of the cells of the fermentation composition of the invention are lysed. Further in the fermentation composition of the invention at least 10%, 25%, 50%, such as at least 75%, such as at least 95%, such as at least 99% of solid cellular material may have been removed and separated from a liquid phase. Moreover, in addition to benzylisoquinoline alkaloid the fermentation composition of the invention may comprise one or more compounds selected from trace metals, vitamins, salts, yeast nitrogen base, carbon source, YNB, and/or amino acids of the fermentation. In particular the fermentation compositing of the invention comprise a concentration of benzylisoquinoline alkaloid is at least 1 mg/kg composition, such as at least 5 mg/kg, such as at least 10 mg/kg, such as at least 20 mg/kg, such as at least 50 mg/kg, such as at least 100 mg/kg, such as at least 500 mg/kg, such as at least 1000 mg/kg, such as at least 5000 mg/kg, such as at least 10000 mg/kg, such as at least 50000 mg/kg.

### Compositions and Use

In a further aspect the invention provides a composition comprising the fermentation composition of the invention (comprising one or more opiate or benzylisoquinoline alkaloid, such as any BIA, BIA-glycoside, oripavine, glucosylated oripavine, gly-oripavine, thebaine, northebaine, nororipavine, gly-nororipavine, glucosylated nororipavine, nor-opioids or glycosylated noropioids) and one or more carriers, agents, additives and/or excipients. Carriers, agents, additives and/or excipients includes formulation additives, stabilising agent, fillers and the like. The composition may be formulated into a dry solid form by using methods known in the art, such as spray drying, spray cooling, lyophilization, flash freezing, granulation, microgranulation, encapsulation or microencapsulation. The composition may also be formulated into liquid stabilized form using methods known in the art, such as formulation into a stabilized liquid comprising one or more stabilizers such as sugars and/or polyols (e.g. sugar alcohols) and/or organic acids (e.g. lactic acid).

Still further the invention provides a pharmaceutical composition comprising the fermentation composition of the invention preceding item and one or more pharmaceutical grade excipient, additives and/or adjuvants. The pharmaceutical composition can be in form of a powder, tablet or capsule, or it can be liquid in the form of a pharmaceutical solution, suspension, lotion or ointment. The pharmaceutical composition can also be incorporated into suitable delivery systems such as for buccal administration or as a patch for transdermal administration.

The invention further provides a method for preparing the pharmaceutical composition of the invention comprising mixing the fermentation composition of the invention with one or more pharmaceutical grade excipient, additives and/or adjuvants.

The pharmaceutical composition is suitably used as a medicament in a method for treating and/or relieving a disease and/or medical condition, in particular in a mammal. Accordingly, the invention further provides a method for preventing, treating and/or relieving a disease and/or medical condition comprising administering a therapeutically effective amount of the pharmaceutical composition of the invention to a mammal in need of treatment and/or relief. Diseases and/or medical conditions treatable or relievable by the pharmaceutical composition includes but is not limited to pain, opiate poisoning conditions, opioid use disorder, alcohol use disorder and/or other conditions. Appropriate and effective dosages of benzylisoquinoline alkaloids are known in the art. The pharmaceutical preparation can be administered parenterally, such as topically, epicutaneously, sublingually, buccally, nasally, intradermally, intralesionally, (intra) ocularly, intravenously, intramuscular, intrapulmonary and/or intravaginally. The pharmaceutical composition can also be administered enterally to the gastrointestinal tract.

### Sequences

The present application contains a Sequence Listing prepared in Patent In ver 3.5 submitted electronically in ST26 format which is hereby incorporated by reference in its entirety. The “SEQ ID NO:” in Table 1 are the numbers used herein. When the terms “Artificial” is used in Table with reference to a nucleic acid sequence, this refers to a nucleic acid sequence that has been codon optimized and/or contains mutations. The following sequences are included:

TABLE 1

| | | | | | |
|---|---|---|---|---|---|
| SEQ ID | Amino acid | DAHP | Aro4fbr | From | Artificial |
| NO: 1 | sequence of | | | | |
| SEQ ID | DNA coding | DAHP | Aro4fbr | From | Artificial |
| NO: 2 | sequence of | | | | |
| SEQ ID | Amino acid | chorismate | ARO7fbr | From | Artificial |
| NO: 3 | sequence of | mutase | | | |
| SEQ ID | DNA coding | chorismate | ARO7fbr | From | Artificial |
| NO: 4 | sequence of | mutase | | | |
| SEQ ID | Amino acid | Tyr1 | Tyr1 | From | S. cerevisiae |
| NO: 5 | sequence of | | | | |
| SEQ ID | DNA coding | Tyr1 | Tyr1 | From | S. cerevisiae |
| NO: 6 | sequence of | | | | |
| SEQ ID | Amino acid | TH | SoCYP76ADr9 | From | Spinacia oleracea |
| NO: 7 | sequence of | | | | |
| SEQ ID | DNA coding | TH | SoCYP76ADr9 | From | Spinacia oleracea |
| NO: 8 | sequence of | | | | |
| SEQ ID | Amino acid | TH | OfCYP76ADr12 | From | Opuntia ficus-indica |
| NO: 9 | sequence of | | | | |
| SEQ ID | DNA coding | TH | OfCYP76ADr12 | From | Opuntia ficus-indica |
| NO: 10 | sequence of | | | | |
| SEQ ID | Amino acid | TH | FICYP76ADr11 | From | Froelichia latifolia |
| NO: 11 | sequence of | | | | |
| SEQ ID | DNA coding | TH | FICYP76ADr11 | From | Froelichia latifolia |
| NO: 12 | sequence of | | | | |
| SEQ ID | Amino acid | TH | BvCYP76ADr10 | From | Beta vulgaris |
| NO: 13 | sequence of | | | | |
| SEQ ID | DNA coding | TH | BvCYP76ADr10 | From | Beta vulgaris |
| NO: 14 | sequence of | | | | |
| SEQ ID | Amino acid | TH | AnCYP76ADr17 | From | Abronia nealleyi |
| NO: 15 | sequence of | | | | |
| SEQ ID | DNA coding | TH | AnCYP76ADr17 | From | Abronia nealleyi |
| NO: 16 | sequence of | | | | |
| SEQ ID | Amino acid | TH | BvCYP76ADr8 | From | Beta vulgaris |
| NO: 17 | sequence of | | | | |
| SEQ ID | DNA coding | TH | BvCYP76ADr8 | From | Beta vulgaris |
| NO: 18 | sequence of | | | | |
| SEQ ID | Amino acid | TH | BvCYP76Ar7 | From | Beta vulgaris |
| NO: 19 | sequence of | | | | |
| SEQ ID | DNA coding | TH | BvCYP76Ar7 | From | Beta vulgaris |
| NO: 20 | sequence of | | | | |
| SEQ ID | Amino acid | TH | BvCYP76ADr6 | From | Beta vulgaris |
| NO: 21 | sequence of | | | | |
| SEQ ID | DNA coding | TH | BvCYP76ADr6 | From | Beta vulgaris |
| NO: 22 | sequence of | | | | |
| SEQ ID | Amino acid | TH | CbCYP76ADr28 | From | Cleretum |
| NO: 23 | sequence of | | | | bellidiforme |
| SEQ ID | DNA coding | TH | CbCYP76ADr28 | From | Cleretum |
| NO: 24 | sequence of | | | | bellidiforme |
| SEQ ID | Amino acid | TH | EvCYP76ADr20 | From | Ercilla volubilis |
| NO: 25 | sequence of | | | | |
| SEQ ID | DNA coding | TH | EvCYP76ADr20 | From | Ercilla volubilis |
| NO: 26 | sequence of | | | | |
| SEQ ID | Amino acid | TH | PdCYP76ADr21 | From | Phytolacca dioica |
| NO: 27 | sequence of | | | | |
| SEQ ID | DNA coding | TH | PdCYP76ADr21 | From | Phytolacca dioica |
| NO: 28 | sequence of | | | | |
| SEQ ID | Amino acid | TH | AoCYP76ADr16 | From | Acleisanthes |
| NO: 29 | sequence of | | | | obtuse |
| SEQ ID | DNA coding | TH | AoCYP76ADr16 | From | Acleisanthes |
| NO: 30 | sequence of | | | | obtuse |
| SEQ ID | Amino acid | TH | MmCYP76ADr18 | From | Mirabilis multiflora |
| NO: 31 | sequence of | | | | |
| SEQ ID | DNA coding | TH | MmCYP76ADr18 | From | Mirabilis multiflora |
| NO: 32 | sequence of | | | | |
| SEQ ID | Amino acid | TH | AoCYP76ADr24 | From | Acleisanthes |
| NO: 33 | sequence of | | | | obtuse |
| SEQ ID | DNA coding | TH | AoCYP76ADr24 | From | Acleisanthes |
| NO: 34 | sequence of | | | | obtuse |
| SEQ ID | Amino acid | TH | AnCYP76ADr27 | From | Abronia nealleyi |
| NO: 35 | sequence of | | | | |
| SEQ ID | DNA coding | TH | AnCYP76ADr27 | From | Abronia nealleyi |
| NO: 36 | sequence of | | | | |
| SEQ ID | Amino acid | TH | PaCYP76ADr19 | From | Phytolacca |
| NO: 37 | sequence of | | | | americana |
| SEQ ID | DNA coding | TH | PaCYP76ADr19 | From | Phytolacca |
| NO: 38 | sequence of | | | | americana |
| SEQ ID | Amino acid | TH | CqCYP76ADr5 | From | Chenopodium |
| NO: 39 | sequence of | | | | quinoa |
| SEQ ID | DNA coding | TH | CqCYP76ADr5 | From | Chenopodium |
| NO: 40 | sequence of | | | | quinoa |
| SEQ ID | Amino acid | TH | MmCYP76ADr22 | From | Mirabilis multiflora |
| NO: 41 | sequence of | | | | |
| SEQ ID | DNA coding | TH | MmCYP76ADr22 | From | Mirabilis multiflora |
| NO: 42 | sequence of | | | | |
| SEQ ID | Amino acid | TH | CqCYP76ADr4 | From | Chenopodium |
| NO: 43 | sequence of | | | | quinoa |
| SEQ ID | DNA coding | TH | CqCYP76ADr4 | From | Chenopodium |
| NO: 44 | sequence of | | | | quinoa |
| SEQ ID | Amino acid | TH | PaCYP76ADr14 | From | Phytolacca |
| NO: 45 | sequence of | | | | americana |
| SEQ ID | DNA coding | TH | PaCYP76ADr14 | From | Phytolacca |
| NO: 46 | sequence of | | | | americana |
| SEQ ID | Amino acid | TH | AnCYP76ADr23 | From | Abronia nealleyi |
| NO: 47 | sequence of | | | | |
| SEQ ID | DNA coding | TH | AnCYP76ADr23 | From | Abronia nealleyi |
| NO: 48 | sequence of | | | | |
| SEQ ID | Amino acid | TH | SoCYP76ADr2 | From | Spinacia oleracea |
| NO: 49 | sequence of | | | | |
| SEQ ID | DNA coding | TH | SoCYP76ADr2 | From | Spinacia oleracea |
| NO: 50 | sequence of | | | | |
| SEQ ID | Amino acid | TH | SoCYP76ADr3 | From | Spinacia oleracea |
| NO: 51 | sequence of | | | | |
| SEQ ID | DNA coding | TH | SoCYP76ADr3 | From | Spinacia oleracea |
| NO: 52 | sequence of | | | | |
| SEQ ID | Amino acid | TH | SoCYP76ADr1 | From | Spinacia oleracea |
| NO: 53 | sequence of | | | | |
| SEQ ID | DNA coding | TH | SoCYP76ADr1 | From | Spinacia oleracea |
| NO: 54 | sequence of | | | | |
| SEQ ID | Amino acid | TH | CqCYP76ADr13 | From | Chenopodium |
| NO: 55 | sequence of | | | | quinoa |
| SEQ ID | DNA coding | TH | CqCYP76ADr13 | From | Chenopodium |
| NO: 56 | sequence of | | | | quinoa |
| SEQ ID | Amino acid | TH | SoCYP76ADr15 | From | Spinacia oleracea |
| NO: 57 | sequence of | | | | |
| SEQ ID | DNA coding | TH | SoCYP76ADr15 | From | Spinacia oleracea |
| NO: 58 | sequence of | | | | |
| SEQ ID | Amino acid | TH | MjCYP76ADr26 | From | Mirabilis jalapa |
| NO: 59 | sequence of | | | | |
| SEQ ID | DNA coding | TH | MjCYP76ADr26 | From | Mirabilis jalapa |
| NO: 60 | sequence of | | | | |
| SEQ ID | Amino acid | TH | MmCYP76ADr25 | From | Mirabilis multiflora |
| NO: 61 | sequence of | | | | |
| SEQ ID | DNA coding | TH | MmCYP76ADr25 | From | Mirabilis multiflora |
| NO: 62 | sequence of | | | | |
| SEQ ID | Amino acid | TH | BvCYP76AD1VM | From | Beta vulgaris |
| NO: 63 | sequence of | | | | |
| SEQ ID | DNA coding | TH | BvCYP76AD1VM | From | Beta vulgaris |
| NO: 64 | sequence of | | | | |
| SEQ ID | Amino acid | TH | CYP76AD1_2mut | From | Artificial |
| NO: 65 | sequence of | | | | |
| SEQ ID | DNA coding | TH | CYP76AD1_2mut | From | Artificial |
| NO: 66 | sequence of | | | | |
| SEQ ID | Amino acid | CPR′′′ | BvCPR1 | From | Beta vulgaris |
| NO: 67 | sequence of | | | | |
| SEQ ID | DNA coding | CPR′′′ | BvCPR1 | From | Beta vulgaris |
| NO: 68 | sequence of | | | | |
| SEQ ID | Amino acid | DoDC | PpDoDC | From | Pseudomonas |
| NO: 69 | sequence of | | | | putida |
| SEQ ID | DNA coding | DoDC | PpDoDC | From | Pseudomonas |
| NO: 70 | sequence of | | | | putida |
| SEQ ID | Amino acid | DoDC | PpDoDC | From | Pseudomonas |
| NO: 71 | sequence of | | | | putida |
| SEQ ID | DNA coding | DoDC | PpDoDC | From | Pseudomonas |
| NO: 72 | sequence of | | | | putida |
| SEQ ID | Amino acid | NCS | d19CjNCS | From | Coptis japonica |
| NO: 73 | sequence of | | | | |
| SEQ ID | DNA coding | NCS | d19CjNCS | From | Coptis japonica |
| NO: 74 | sequence of | | | | |
| SEQ ID | DNA coding | NCS | d19CjNCS | From | Coptis japonica |
| NO: 75 | sequence of | | | | |
| SEQ ID | Amino acid | NCS | HDEL_CjNCS_V152 | From | Artificial |
| NO: 76 | sequence of | | | | |
| SEQ ID | DNA coding | NCS | HDEL_CjNCS_V152 | From | Artificial |
| NO: 77 | sequence of | | | | |
| SEQ ID | DNA coding | Integration | pRIV40 | From | Artificial |
| NO: 78 | sequence of | plasmid | | | |
| SEQ ID | Amino acid | 6-OMT | Ps6OMT_Q6WUC1 | From | Papaver |
| NO: 79 | sequence of | | | | somniferum |
| SEQ ID | DNA coding | 6-OMT | Ps6OMT_Q6WUC1 | From | Papaver |
| NO: 80 | sequence of | | | | somniferum |
| SEQ ID | Amino acid | 6-OMT | — | From | Papaver |
| NO: 81 | sequence of | | | | somniferum |
| SEQ ID | Amino acid | CNMT | CjCNMT | From | Coptis japonica |
| NO: 82 | sequence of | | | | |
| SEQ ID | DNA coding | CNMT | CjCNMT | From | Coptis japonica |
| NO: 83 | sequence of | | | | |
| SEQ ID | Amino acid | CNMT | — | From | Papaver |
| NO: 84 | sequence of | | | | somniferum |
| SEQ ID | Amino acid | NMCH | EcNMCH | From | Eschscholzia |
| NO: 85 | sequence of | | | | californica |
| SEQ ID | DNA coding | NMCH | EcNMCH | From | Eschscholzia |
| NO: 86 | sequence of | | | | californica |
| SEQ ID | Amino acid | NMCH | — | From | Eschscholzia |
| NO: 87 | sequence of | | | | californica |
| SEQ ID | DNA coding | NMCH | — | From | Eschscholzia |
| NO: 88 | sequence of | | | | californica |
| SEQ ID | Amino acid | 4′-OMT | Cj4OMT | From | Coptis japonica |
| NO: 89 | sequence of | | | | |
| SEQ ID | DNA coding | 4′-OMT | Cj4OMT | From | Coptis japonica |
| NO: 90 | sequence of | | | | |
| SEQ ID | Amino acid | 4′-OMT | — | From | Papaver |
| NO: 91 | sequence of | | | | somniferum |
| SEQ ID | Amino acid | STORR | DRS-DRR | From | Papaver |
| NO: 92 | sequence of | | | | bracteatum |
| SEQ ID | DNA coding | STORR | DRS-DRR | From | Papaver |
| NO: 93 | sequence of | | | | bracteatum |
| SEQ ID | Amino acid | STORR | StlRED | From | Streptomyces |
| NO: 94 | sequence of | | | | tsukubaensis |
| SEQ ID | DNA coding | STORR | StlRED | From | Streptomyces |
| NO: 95 | sequence of | | | | tsukubaensis |
| SEQ ID | Amino acid | STORR | PsSTORR | From | Papaver |
| NO: 96 | sequence of | | | | somniferum |
| SEQ ID | DNA coding | STORR | PsSTORR | From | Papaver |
| NO: 97 | sequence of | | | | somniferum |
| SEQ ID | Amino acid | STORR | PsCYP82Y2 | From | Papaver |
| NO: 98 | sequence of | P450 | | | somniferum |
| SEQ ID | DNA coding | STORR | PsCYP82Y2 | From | Papaver |
| NO: 99 | sequence of | P450 | | | somniferum |
| SEQ ID | Amino acid | STORR | PrCYP82Y2-like | From | Papaver rhoeas |
| NO: 100 | sequence of | P450 | | | |
| SEQ ID | DNA coding | STORR | PrCYP82Y2-like | From | Papaver rhoeas |
| NO: 101 | sequence of | P450 | | | |
| SEQ ID | Amino acid | STORR | proID60 | From | Artificial |
| NO: 102 | sequence of | P450 | | | |
| SEQ ID | DNA coding | STORR | proID60 | From | Artificial |
| NO: 103 | sequence of | P450 | | | |
| SEQ ID | Amino acid | STORR | proID66 | From | Artificial |
| NO: 104 | sequence of | P450 | | | |
| SEQ ID | DNA coding | STORR | proID66 | From | Artificial |
| NO: 105 | sequence of | P450 | | | |
| SEQ ID | Amino acid | STORR | proID79 | From | Artificial |
| NO: 106 | sequence of | P450 | | | |
| SEQ ID | DNA coding | STORR | proID79 | From | Artificial |
| NO: 107 | sequence of | P450 | | | |
| SEQ ID | Amino acid | STORR | PsAKR | From | Papaver |
| NO: 108 | sequence of | Reductase | | | somniferum |
| SEQ ID | DNA coding | STORR | PsAKR | From | Papaver |
| NO: 109 | sequence of | Reductase | | | somniferum |
| SEQ ID | Amino acid | STORR | PrAKR | From | Papaver rhoeas |
| NO: 110 | sequence of | Reductase | | | |
| SEQ ID | DNA coding | STORR | PrAKR | From | Papaver rhoeas |
| NO: 111 | sequence of | Reductase | | | |
| SEQ ID | Amino acid | CPR″ | PsCPR | From | Papaver |
| NO: 112 | sequence of | | | | somniferum |
| SEQ ID | DNA coding | CPR″ | PsCPR | From | Papaver |
| NO: 113 | sequence of | | | | somniferum |
| SEQ ID | Amino acid | CPR″ | AtATR1 | From | Arabidopsis |
| NO: 114 | sequence of | | | | thaliana |
| SEQ ID | DNA coding | CPR″ | AtATR1 | From | Arabidopsis |
| NO: 115 | sequence of | | | | thaliana |
| SEQ ID | Amino acid | SAS | PbSAS | From | Papaver |
| NO: 116 | sequence of | | | | bracteatum |
| SEQ ID | DNA coding | SAS | PbSAS | From | Papaver |
| NO: 117 | sequence of | | | | bracteatum |
| SEQ ID | Amino acid | SAS | — | From | Papaver |
| NO: 118 | sequence of | | | | bracteatum |
| SEQ ID | DNA coding | SAS | — | From | Papaver |
| NO: 119 | sequence of | | | | bracteatum |
| SEQ ID | Amino acid | SAR | pbSalR | From | Papaver |
| NO: 120 | sequence of | | | | bracteatum |
| SEQ ID | DNA coding | SAR | pbSalR | From | Papaver |
| NO: 121 | sequence of | | | | bracteatum |
| SEQ ID | Amino acid | SAR | — | From | Papaver |
| NO: 122 | sequence of | | | | bracteatum |
| SEQ ID | Amino acid | SAT | PsSAT | From | Papaver |
| NO: 123 | sequence of | | | | somniferum |
| SEQ ID | DNA coding | SAT | PsSAT | From | Papaver |
| NO: 124 | sequence of | | | | somniferum |
| SEQ ID | Amino acid | SAT | — | From | Papaver |
| NO: 125 | sequence of | | | | somniferum |
| SEQ ID | Amino acid | THS | HA BetV1M | From | Papaver |
| NO: 126 | sequence of | | | | somniferum |
| SEQ ID | Amino acid | THS | BETV1L HA | From | Papaver |
| NO: 127 | sequence of | | | | somniferum |
| SEQ ID | Amino acid | THS | — | From | Papaver |
| NO: 128 | sequence of | | | | somniferum |
| SEQ ID | Amino acid | THS | PsTHS1 | From | Papaver |
| NO: 129 | sequence of | | | | somniferum |
| SEQ ID | DNA coding | THS | PsTHS1 | From | Papaver |
| NO: 130 | sequence of | | | | somniferum |
| SEQ ID | Amino acid | THS | PsTHS2 | From | Papaver |
| NO: 131 | sequence of | | | | somniferum |
| SEQ ID | DNA coding | THS | PsTHS2 | From | Papaver |
| NO: 132 | sequence of | | | | somniferum |
| SEQ ID | Amino acid | THS | — | From | Papaver |
| NO: 133 | sequence of | | | | somniferum |
| SEQ ID | Amino acid | THS | PROths2_138 | From | Artificial |
| NO: 134 | sequence of | | | | |
| SEQ ID | DNA coding | THS | PROths2_138 | From | Artificial |
| NO: 135 | sequence of | | | | |
| SEQ ID | Amino acid | THS | PROths2_143 | From | Artificial |
| NO: 136 | sequence of | | | | |
| SEQ ID | DNA coding | THS | PROths2_143 | From | Artificial |
| NO: 137 | sequence of | | | | |
| SEQ ID | Amino acid | THS | PROths2_116 | From | Artificial |
| NO: 138 | sequence of | | | | |
| SEQ ID | DNA coding | THS | PROths2_116 | From | Artificial |
| NO: 139 | sequence of | | | | |
| SEQ ID | Amino acid | P450 | HaCYP6AE15v2 | From | Helicoverpa |
| NO: 140 | sequence of | | protein | | armigera |
| SEQ ID | DNA coding | P450 | HaCYP6AE15v2 | From | Helicoverpa |
| NO: 141 | sequence of | | *DNA | | armigera |
| SEQ ID | Amino acid | P450 | HaCYP6AE19 protein | From | Helicoverpa |
| NO: 142 | sequence of | | | | armigera |
| SEQ ID | DNA coding | P450 | HaCYP6AE19 *DNA | From | Helicoverpa |
| NO: 143 | sequence of | | | | armigera |
| SEQ ID | Amino acid | P450 | HaCYP6AE11 protein | From | Helicoverpa |
| NO: 144 | sequence of | | | | armigera |
| SEQ ID | DNA coding | P450 | HaCYP6AE11 *DNA | From | Helicoverpa |
| NO: 145 | sequence of | | | | armigera |
| SEQ ID | Amino acid | P450 | HaCYP6AE17 protein | From | Helicoverpa |
| NO: 146 | sequence of | | | | armigera |
| SEQ ID | DNA coding | P450 | HaCYP6AE17 *DNA | From | Helicoverpa |
| NO: 147 | sequence of | | | | armigera |
| SEQ ID | Amino acid | P450 | HaCYP6AE24 protein | From | Helicoverpa |
| NO: 148 | sequence of | | | | armigera |
| SEQ ID | DNA coding | P450 | HaCYP6AE24 *DNA | From | Helicoverpa |
| NO: 149 | sequence of | | | | armigera |
| SEQ ID | Amino acid | P450 | HaCYP6AE20v2 | From | Helicoverpa |
| NO: 150 | sequence of | | protein | | armigera |
| SEQ ID | DNA coding | P450 | HaCYP6AE20v2 | From | Helicoverpa |
| NO: 151 | sequence of | | *DNA | | armigera |
| SEQ ID | Amino acid | P450 | Hv_CYP_A0A2A4JAM9 | From | Heliothis virescens |
| NO: 152 | sequence of | | protein | | |
| SEQ ID | DNA coding | P450 | Hv_CYP_A0A2A4JAM9 | From | Heliothis virescens |
| NO: 153 | sequence of | | *DNA | | |
| SEQ ID | Amino acid | P450 | Hv_CYP_A0A2A4JAK3 | From | Heliothis virescens |
| NO: 154 | sequence of | | protein | | |
| SEQ ID | DNA coding | P450 | Hv_CYP_A0A2A4JAK3 | From | Heliothis virescens |
| NO: 155 | sequence of | | *DNA | | |
| SEQ ID | Amino acid | P450 | Se_CYP6AE68 | From | Spodoptera exigua |
| NO: 156 | sequence of | | protein | | |
| SEQ ID | DNA coding | P450 | Se_CYP6AE68 | From | Spodoptera exigua |
| NO: 157 | sequence of | | *DNA | | |
| SEQ ID | Amino acid | P450 | Hv_CYP_A0A2A4J7V4 | From | Heliothis virescens |
| NO: 158 | sequence of | | protein | | |
| SEQ ID | DNA coding | P450 | Hv_CYP_A0A2A4J7V4 | From | Heliothis virescens |
| NO: 159 | sequence of | | *DNA | | |
| SEQ ID | Amino acid | P450 | CmCYP6_A0A0C5CGV6 | From | Cnaphalocrocis |
| NO: 160 | sequence of | | protein | | medinalis |
| SEQ ID | DNA coding | P450 | CmCYP6_A0A0C5CGV6 | From | Cnaphalocrocis |
| NO: 161 | sequence of | | *DNA | | medinalis |
| SEQ ID | Amino acid | P450 | BmCYP6AE9_A9QW15 | From | Bombyx mandarina |
| NO: 162 | sequence of | | protein | | |
| SEQ ID | DNA coding | P450 | BmCYP6AE9_A9QW15 | From | Bombyx mandarina |
| NO: 163 | sequence of | | *DNA | | |
| SEQ ID | Amino acid | P450 | Bm_CYP6AE9 | From | Bombyx mori |
| NO: 164 | sequence of | | protein | | |
| SEQ ID | DNA coding | P450 | Bm_CYP6AE9 *DNA | From | Bombyx mori |
| NO: 165 | sequence of | | | | |
| SEQ ID | Amino acid | P450 | Se_CYP6AE10 | From | Spodoptera exigua |
| NO: 166 | sequence of | | protein | | |
| SEQ ID | DNA coding | P450 | Se_CYP6AE10 *DNA | From | Spodoptera exigua |
| NO: 167 | sequence of | | | | |
| SEQ ID | Amino acid | P450 | Sf_CYP_A0A2H1WID4 | From | Spodoptera |
| NO: 168 | sequence of | | protein | | frugiperda |
| SEQ ID | DNA coding | P450 | Sf_CYP_A0A2H1WID4 | From | Spodoptera |
| NO: 169 | sequence of | | *DNA | | frugiperda |
| SEQ ID | Amino acid | P450 | Sf_CYP_A0A2H1V0E7 | From | Spodoptera |
| NO: 170 | sequence of | | protein | | frugiperda |
| SEQ ID | DNA coding | P450 | Sf_CYP_A0A2H1V0E7 | From | Spodoptera |
| NO: 171 | sequence of | | *DNA | | frugiperda |
| SEQ ID | Amino acid | P450 | Ha_CYP6AE12 | From | Helicoverpa |
| NO: 172 | sequence of | | protein | | armigera |
| SEQ ID | DNA coding | P450 | Ha_CYP6AE12 *DNA | From | Helicoverpa |
| NO: 173 | sequence of | | | | armigera |
| SEQ ID | Amino acid | P450 | Sf_CYP6AE44 | From | Spodoptera |
| NO: 174 | sequence of | | protein | | frugiperda |
| SEQ ID | DNA coding | P450 | Sf_CYP6AE44 *DNA | From | Spodoptera |
| NO: 175 | sequence of | | | | frugiperda |
| SEQ ID | Amino acid | P450 | HaCYP6AE_A0A068F0X7 | From | Helicoverpa |
| NO: 176 | sequence of | | protein | | armigera |
| SEQ ID | DNA coding | P450 | HaCYP6AE_A0A068F0X7 | From | Helicoverpa |
| NO: 177 | sequence of | | *DNA | | armigera |
| SEQ ID | Amino acid | P450 | DpCYP_Q7YZS2 | From | Depressaria |
| NO: 178 | sequence of | | protein | | pastinacella |
| SEQ ID | DNA coding | P450 | DpCYP_Q7YZS2 | From | Depressaria |
| NO: 179 | sequence of | | *DNA | | pastinacella |
| SEQ ID | Amino acid | P450 | Sf_CYP_A0A2H1V0E7 | From | Spodoptera |
| NO: 180 | sequence of | | protein | | frugiperda |
| SEQ ID | DNA coding | P450 | Sf_CYP_A0A2H1V0E7 | From | Spodoptera |
| NO: 181 | sequence of | | *DNA | | frugiperda |
| SEQ ID | Amino acid | P450 | BmCYP6AE2_L0N7C5 | From | Bombyx mori |
| NO: 182 | sequence of | | protein | | |
| SEQ ID | DNA coding | P450 | BmCYP6AE2_L0N7C5 | From | Bombyx mori |
| NO: 183 | sequence of | | *DNA | | |
| SEQ ID | Amino acid | P450 | BmCYP_C1KJL7 | From | Bombyx mandarina |
| NO: 184 | sequence of | | protein | | |
| SEQ ID | DNA coding | P450 | BmCYP_C1KJL7 | From | Bombyx mandarina |
| NO: 185 | sequence of | | *DNA | | |
| SEQ ID | Amino acid | P450 | ZfCYP6AE27_D2JLK6 | From | Zygaena |
| NO: 186 | sequence of | | protein | | filipendulae |
| SEQ ID | DNA coding | P450 | ZfCYP6AE27_D2JLK6 | From | Zygaena |
| NO: 187 | sequence of | | *DNA | | filipendulae |
| SEQ ID | Amino acid | P450 | BmCyp6AE21_B6VFR9 | From | Bombyx mori |
| NO: 188 | sequence of | | protein | | |
| SEQ ID | DNA coding | P450 | BmCyp6AE21_B6VFR9 | From | Bombyx mori |
| NO: 189 | sequence of | | *DNA | | |
| SEQ ID | Amino acid | P450 | BmCYP6AE7_A4GUB8 | From | Bombyx mori |
| NO: 190 | sequence of | | protein | | |
| SEQ ID | DNA coding | P450 | BmCYP6AE7_A4GUB8 | From | Bombyx mori |
| NO: 191 | sequence of | | *DNA | | |
| SEQ ID | Amino acid | P450 | CmCYP6_A0A0C5C1l6 | From | Cnaphalocrocis |
| NO: 192 | sequence of | | protein | | medinalis |
| SEQ ID | DNA coding | P450 | CmCYP6_A0A0C5C1l6 | From | Cnaphalocrocis |
| NO: 193 | sequence of | | *DNA | | medinalis |
| SEQ ID | Amino acid | P450 | SeCYP6_A0A248QEH8 | From | Spodoptera exigua |
| NO: 194 | sequence of | | protein | | |
| SEQ ID | DNA coding | P450 | SeCYP6_A0A248QEH8 | From | Spodoptera exigua |
| NO: 195 | sequence of | | *DNA | | |
| SEQ ID | Amino acid | P450 | BmCYP6AE9_A5HKM1 | From | Bombyx mori |
| NO: 196 | sequence of | | protein | | |
| SEQ ID | DNA coding | P450 | BmCYP6AE9_A5HKM1 | From | Bombyx mori |
| NO: 197 | sequence of | | *DNA | | |
| SEQ ID | Amino acid | P450 | CYPDN_39 protein | From | Rhizopus |
| NO: 198 | sequence of | | | | microsporus |
| SEQ ID | DNA coding | P450 | CYPDN_39 gene | From | Rhizopus |
| NO: 199 | sequence of | | | | microsporus |
| SEQ ID | Amino acid | P450 | CYPDN_41 protein | From | Rhizopus |
| NO: 200 | sequence of | | | | microsporus |
| SEQ ID | DNA coding | P450 | CYPDN_41 gene | From | Rhizopus |
| NO: 201 | sequence of | | | | microsporus |
| SEQ ID | Amino acid | P450 | CYPDN_43 protein | From | Lichtheimia |
| NO: 202 | sequence of | | | | corymbifera |
| SEQ ID | DNA coding | P450 | CYPDN_43 gene | From | Lichtheimia |
| NO: 203 | sequence of | | | | corymbifera |
| SEQ ID | Amino acid | P450 | CYPDN_44 protein | From | Lichtheimia ramosa |
| NO: 204 | sequence of | | | | |
| SEQ ID | DNA coding | P450 | CYPDN_44 gene | From | Lichtheimia ramosa |
| NO: 205 | sequence of | | | | |
| SEQ ID | Amino acid | P450 | CYPDN_45 protein | From | Rhizopus |
| NO: 206 | sequence of | | | | microsporus |
| SEQ ID | DNA coding | P450 | CYPDN_45 gene | From | Rhizopus |
| NO: 207 | sequence of | | | | microsporus |
| SEQ ID | Amino acid | P450 | CYPDN_50 protein | From | Lichtheimia ramosa |
| NO: 208 | sequence of | | | | |
| SEQ ID | DNA coding | P450 | CYPDN_50 gene | From | Lichtheimia ramosa |
| NO: 209 | sequence of | | | | |
| SEQ ID | Amino acid | P450 | CYPDN_51 protein | From | Lichtheimia ramosa |
| NO: 210 | sequence of | | | | |
| SEQ ID | DNA coding | P450 | CYPDN_51 gene | From | Lichtheimia ramosa |
| NO: 211 | sequence of | | | | |
| SEQ ID | Amino acid | P450 | CYPDN_57 protein | From | Syncephalastrum |
| NO: 212 | sequence of | | | | racemosum |
| SEQ ID | DNA coding | P450 | CYPDN_57 gene | From | Syncephalastrum |
| NO: 213 | sequence of | | | | racemosum |
| SEQ ID | Amino acid | P450 | CYPDN_59 protein | From | Cunninghamella |
| NO: 214 | sequence of | | | | echinulata |
| SEQ ID | DNA coding | P450 | CYPDN_59 gene | From | Cunninghamella |
| NO: 215 | sequence of | | | | echinulata |
| SEQ ID | Amino acid | P450 | CYPDN_61 protein | From | Rhizopus |
| NO: 216 | sequence of | | | | azygosporus |
| SEQ ID | DNA coding | P450 | CYPDN_61 gene | From | Rhizopus |
| NO: 217 | sequence of | | | | azygosporus |
| SEQ ID | Amino acid | P450 | CYPDN_62 protein | From | Rhizopus |
| NO: 218 | sequence of | | | | azygosporus |
| SEQ ID | DNA coding | P450 | CYPDN_62 gene | From | Rhizopus |
| NO: 219 | sequence of | | | | azygosporus |
| SEQ ID | Amino acid | P450 | CYPDN_63 protein | From | Rhizopus |
| NO: 220 | sequence of | | | | microsporus |
| SEQ ID | DNA coding | P450 | CYPDN_63 gene | From | Rhizopus |
| NO: 221 | sequence of | | | | microsporus |
| SEQ ID | Amino acid | P450 | CYPDN_64 protein | From | Mucor circinelloides |
| NO: 222 | sequence of | | | | f. circinelloides |
| SEQ ID | DNA coding | P450 | CYPDN_64 gene | From | Mucor circinelloides |
| NO: 223 | sequence of | | | | f. circinelloides |
| SEQ ID | Amino acid | P450 | CYPDN_65 protein | From | Mucor ambiguus |
| NO: 224 | sequence of | | | | |
| SEQ ID | DNA coding | P450 | CYPDN_65 gene | From | Mucor ambiguus |
| NO: 225 | sequence of | | | | |
| SEQ ID | Amino acid | P450 | CYPDN_67 protein | From | Syncephalastrum |
| NO: 226 | sequence of | | | | racemosum |
| SEQ ID | DNA coding | P450 | CYPDN_67 gene | From | Syncephalastrum |
| NO: 227 | sequence of | | | | racemosum |
| SEQ ID | Amino acid | P450 | CYPDN_68 protein | From | Parasitella |
| NO: 228 | sequence of | | | | parasitica |
| SEQ ID | DNA coding | P450 | CYPDN_68 gene | From | Parasitella |
| NO: 229 | sequence of | | | | parasitica |
| SEQ ID | Amino acid | P450 | CYPDN_69 protein | From | Syncephalastrum |
| NO: 230 | sequence of | | | | racemosum |
| SEQ ID | DNA coding | P450 | CYPDN_69 gene | From | Syncephalastrum |
| NO: 231 | sequence of | | | | racemosum |
| SEQ ID | Amino acid | P450 | CYPDN_70 protein | From | Lichtheimia ramosa |
| NO: 232 | sequence of | | | | |
| SEQ ID | DNA coding | P450 | CYPDN_70 gene | From | Lichtheimia ramosa |
| NO: 233 | sequence of | | | | |
| SEQ ID | Amino acid | P450 | CYPDN_74 protein | From | Lichtheimia |
| NO: 234 | sequence of | | | | corymbifera |
| SEQ ID | DNA coding | P450 | CYPDN_74 gene | From | Lichtheimia |
| NO: 235 | sequence of | | | | corymbifera |
| SEQ ID | Amino acid | P450 | CYPDN_75 protein | From | Absidia repens |
| NO: 236 | sequence of | | | | |
| SEQ ID | DNA coding | P450 | CYPDN_75 gene | From | Absidia repens |
| NO: 237 | sequence of | | | | |
| SEQ ID | Amino acid | P450 | CYPDN_77 protein | From | Lichtheimia |
| NO: 238 | sequence of | | | | corymbifera |
| SEQ ID | DNA coding | P450 | CYPDN_77 gene | From | Lichtheimia |
| NO: 239 | sequence of | | | | corymbifera |
| SEQ ID | Amino acid | P450 | CYPDN_80 protein | From | Absidia glauca |
| NO: 240 | sequence of | | | | |
| SEQ ID | DNA coding | P450 | CYPDN_80 gene | From | Absidia glauca |
| NO: 241 | sequence of | | | | |
| SEQ ID | Amino acid | P450 | CYPDN_82 protein | From | Choanephora |
| NO: 242 | sequence of | | | | cucurbitarum |
| SEQ ID | DNA coding | P450 | CYPDN_82 gene | From | Choanephora |
| NO: 243 | sequence of | | | | cucurbitarum |
| SEQ ID | Amino acid | P450 | CYPDN_84 protein | From | Absidia glauca |
| NO: 244 | sequence of | | | | |
| SEQ ID | DNA coding | P450 | CYPDN_84 gene | From | Absidia glauca |
| NO: 245 | sequence of | | | | |
| SEQ ID | Amino acid | P450 | CYPDN_85 protein | From | Absidia repens |
| NO: 246 | sequence of | | | | |
| SEQ ID | DNA coding | P450 | CYPDN_85 gene | From | Absidia repens |
| NO: 247 | sequence of | | | | |
| SEQ ID | Amino acid | P450 | CYPDN_86 protein | From | Absidia repens |
| NO: 248 | sequence of | | | | |
| SEQ ID | DNA coding | P450 | CYPDN_86 gene | From | Absidia repens |
| NO: 249 | sequence of | | | | |
| SEQ ID | Amino acid | P450 | CYPDN_91 protein | From | Rhizopus |
| NO: 250 | sequence of | | | | microsporus |
| SEQ ID | DNA coding | P450 | CYPDN_91 gene | From | Rhizopus |
| NO: 251 | sequence of | | | | microsporus |
| SEQ ID | Amino acid | P450 | CYPDN_92 protein | From | Rhizopus |
| NO: 252 | sequence of | | | | azygosporus |
| SEQ ID | DNA coding | P450 | CYPDN_92 gene | From | Rhizopus |
| NO: 253 | sequence of | | | | azygosporus |
| SEQ ID | Amino acid | P450 | CYPDN_93 protein | From | Rhizopus |
| NO: 254 | sequence of | | | | azygosporus |
| SEQ ID | DNA coding | P450 | CYPDN_93 gene | From | Rhizopus |
| NO: 255 | sequence of | | | | azygosporus |
| SEQ ID | Amino acid | P450 | CYPDN_95 protein | From | Bifiguratus |
| NO: 256 | sequence of | | | | adelaidae |
| SEQ ID | DNA coding | P450 | CYPDN_95 gene | From | Bifiguratus |
| NO: 257 | sequence of | | | | adelaidae |
| SEQ ID | Amino acid | P450 | CYPDN_98 protein | From | Rhizopus stolonifer |
| NO: 258 | sequence of | | | | |
| SEQ ID | DNA coding | P450 | CYPDN_98 gene | From | Rhizopus stolonifer |
| NO: 259 | sequence of | | | | |
| SEQ ID | Amino acid | P450 | CYPDN_100 protein | From | Rhizopus oryzae |
| NO: 260 | sequence of | | | | |
| SEQ ID | DNA coding | P450 | CYPDN_100 gene | From | Rhizopus oryzae |
| NO: 261 | sequence of | | | | |
| SEQ ID | Amino acid | P450 | CYPDN_101 protein | From | Rhizopus |
| NO: 262 | sequence of | | | | microsporus |
| SEQ ID | DNA coding | P450 | CYPDN_101 gene | From | Rhizopus |
| NO: 263 | sequence of | | | | microsporus |
| SEQ ID | Amino acid | P450 | CYPDN_103 protein | From | Rhizopus delemar |
| NO: 264 | sequence of | | | | RA 99-880 |
| SEQ ID | DNA coding | P450 | CYPDN_103 gene | From | Rhizopus delemar |
| NO: 265 | sequence of | | | | RA 99-880 |
| SEQ ID | Amino acid | P450 | CYPDN_104 protein | From | Rhizopus stolonifer |
| NO: 266 | sequence of | | | | |
| SEQ ID | DNA coding | P450 | CYPDN_104 gene | From | Rhizopus stolonifer |
| NO: 267 | sequence of | | | | |
| SEQ ID | Amino acid | P450 | CYPDN_105 protein | From | Rhizopus |
| NO: 268 | sequence of | | | | azygosporus |
| SEQ ID | DNA coding | P450 | CYPDN_105 gene | From | Rhizopus |
| NO: 269 | sequence of | | | | azygosporus |
| SEQ ID | Amino acid | P450 | CYPDN_108 protein | From | Mucor circinelloides |
| NO: 270 | sequence of | | | | f. circinelloides |
| SEQ ID | DNA coding | P450 | CYPDN_108 gene | From | Mucor circinelloides |
| NO: 271 | sequence of | | | | f. circinelloides |
| SEQ ID | Amino acid | P450 | CYPDN_109 protein | From | Mucor circinelloides |
| NO: 272 | sequence of | | | | f. circinelloides |
| SEQ ID | DNA coding | P450 | CYPDN_109 gene | From | Mucor circinelloides |
| NO: 273 | sequence of | | | | f. circinelloides |
| SEQ ID | Amino acid | P450 | CYPDN_110 protein | From | Mucor circinelloides |
| NO: 274 | sequence of | | | | f. lusitanicus |
| SEQ ID | DNA coding | P450 | CYPDN_110 gene | From | Mucor circinelloides |
| NO: 275 | sequence of | | | | f. lusitanicus |
| SEQ ID | Amino acid | P450 | CYPDN_112 protein | From | Choanephora |
| NO: 276 | sequence of | | | | cucurbitarum |
| SEQ ID | DNA coding | P450 | CYPDN_112 gene | From | Choanephora |
| NO: 277 | sequence of | | | | cucurbitarum |
| SEQ ID | Amino acid | P450 | CYPDN_115 protein | From | Lichtheimia |
| NO: 278 | sequence of | | | | corymbifera |
| SEQ ID | DNA coding | P450 | CYPDN_115 gene | From | Lichtheimia |
| NO: 279 | sequence of | | | | corymbifera |
| SEQ ID | Amino acid | P450 | CYPDN_117 protein | From | Lichtheimia |
| NO: 280 | sequence of | | | | corymbifera |
| SEQ ID | DNA coding | P450 | CYPDN_117 gene | From | Lichtheimia |
| NO: 281 | sequence of | | | | corymbifera |
| SEQ ID | Amino acid | P450 | CYPDN_118 protein | From | Lichtheimia ramosa |
| NO: 282 | sequence of | | | | |
| SEQ ID | DNA coding | P450 | CYPDN_118 gene | From | Lichtheimia ramosa |
| NO: 283 | sequence of | | | | |
| SEQ ID | Amino acid | P450 | CYPDN_119 protein | From | Lichtheimia |
| NO: 284 | sequence of | | | | corymbifera |
| SEQ ID | DNA coding | P450 | CYPDN_119 gene | From | Lichtheimia |
| NO: 285 | sequence of | | | | corymbifera |
| SEQ ID | Amino acid | P450 | CYPDN_120 protein | From | Lichtheimia ramosa |
| NO: 286 | sequence of | | | | |
| SEQ ID | DNA coding | P450 | CYPDN_120 gene | From | Lichtheimia ramosa |
| NO: 287 | sequence of | | | | |
| SEQ ID | Amino acid | P450 | CYPDN_123 protein | From | Lichtheimia ramosa |
| NO: 288 | sequence of | | | | |
| SEQ ID | DNA coding | P450 | CYPDN_123 gene | From | Lichtheimia ramosa |
| NO: 289 | sequence of | | | | |
| SEQ ID | Amino acid | P450 | CYPDN_8 protein | From | Rhizopus |
| NO: 290 | sequence of | | | | microsporus |
| SEQ ID | DNA coding | P450 | CYPDN_8 *DNA | From | Rhizopus |
| NO: 291 | sequence of | | | | microsporus |
| SEQ ID | Amino acid | CPR′ | HaCPR_E0A3A7 | From | Helicoverpa |
| NO: 292 | sequence of | | protein | | armigera |
| SEQ ID | DNA coding | CPR′ | HaCPR_E0A3A7 | From | Helicoverpa |
| NO: 293 | sequence of | | *DNA | | armigera |
| SEQ ID | Amino acid | CPR′ | Se_CPR_F1DI27 | From | Spodoptera exigua |
| NO: 294 | sequence of | | | | |
| SEQ ID | DNA coding | CPR′ | Se_CPR_F1DI27 | From | Spodoptera exigua |
| NO: 295 | sequence of | | | | |
| SEQ ID | Amino acid | CPR′ | Bm_CPR_Q9NKV3 | From | Bombyx mori |
| NO: 296 | sequence of | | | | |
| SEQ ID | DNA coding | CPR′ | Bm_CPR_Q9NKV3 | From | Bombyx mori |
| NO: 297 | sequence of | | | | |
| SEQ ID | Amino acid | CPR′ | BmCPR_A0FGR6 | From | Bombyx mandarina |
| NO: 298 | sequence of | | | | |
| SEQ ID | DNA coding | CPR′ | BmCPR_A0FGR6 | From | Bombyx mandarina |
| NO: 299 | sequence of | | | | |
| SEQ ID | Amino acid | CPR′ | ZfCPR_A0A346M705 | From | Zygaena |
| NO: 300 | sequence of | | | | filipendulae |
| SEQ ID | DNA coding | CPR′ | ZfCPR_A0A346M705 | From | Zygaena |
| NO: 301 | sequence of | | | | filipendulae |
| SEQ ID | Amino acid | CPR′ | CmCPR_A0A1S5ZY34 | From | Cnaphalocrocis |
| NO: 302 | sequence of | | | | medinalis |
| SEQ ID | DNA coding | CPR′ | CmCPR_A0A1S5ZY34 | From | Cnaphalocrocis |
| NO: 303 | sequence of | | | | medinalis |
| SEQ ID | DNA coding | CPR′ | HaCPR_E7E2N6 | From | Helicoverpa |
| NO: 304 | sequence of | | | | armigera |
| SEQ ID | Amino acid | CPR′ | CeCPR protein | From | Cunninghamella |
| NO: 305 | sequence of | | | | elegans |
| SEQ ID | DNA coding | CPR′ | CeCPR gene | From | Cunninghamella |
| NO: 306 | sequence of | | | | elegans |
| SEQ ID | Amino acid | Uptake | T1_CjaMDR1_GA | From | Camellia japonica |
| NO: 307 | sequence of | Transporter | | | |
| SEQ ID | DNA coding | Uptake | T1_CjaMDR1_GA | From | Camellia japonica |
| NO: 308 | sequence of | Transporter | | | |
| SEQ ID | Amino acid | Uptake | T3_NcaNPF_GA | From | Noccaea |
| NO: 309 | sequence of | Transporter | | | caerulescens |
| SEQ ID | DNA coding | Uptake | T3_NcaNPF_GA | From | Noccaea |
| NO: 310 | sequence of | Transporter | | | caerulescens |
| SEQ ID | Amino acid | Uptake | T4_EsaGTR_GA | From | Eutrema |
| NO: 311 | sequence of | Transporter | | | salsugineum |
| SEQ ID | DNA coding | Uptake | T4_EsaGTR_GA | From | Eutrema |
| NO: 312 | sequence of | Transporter | | | salsugineum |
| SEQ ID | Amino acid | Uptake | T5_AlyPOT_GA | From | Arabidopsis lyrata |
| NO: 313 | sequence of | Transporter | | | subsp. lyrata |
| SEQ ID | DNA coding | Transporter | T5_AlyPOT_GA | From | Arabidopsis lyrata |
| NO: 314 | sequence of | | | | subsp. lyrata |
| SEQ ID | Amino acid | Uptake | T6_CruGTR_GA | From | Capsella rubella |
| NO: 315 | sequence of | Transporter | | | |
| SEQ ID | DNA coding | Uptake | T6_CruGTR_GA | From | Capsella rubella |
| NO: 316 | sequence of | Transporter | | | |
| SEQ ID | Amino acid | Uptake | T7_PtrPOT_GA | From | Populus |
| NO: 317 | sequence of | Transporter | | | trichocarpa |
| SEQ ID | DNA coding | Uptake | T7_PtrPOT_GA | From | Populus |
| NO: 318 | sequence of | Transporter | | | trichocarpa |
| SEQ ID | Amino acid | Uptake | T8_BnaMFS_GA | From | Brassica napus |
| NO: 319 | sequence of | Transporter | | | |
| SEQ ID | DNA coding | Uptake | T8_BnaMFS_GA | From | Brassica napus |
| NO: 320 | sequence of | Transporter | | | |
| SEQ ID | Amino acid | Uptake | T10_BolGTR_GA | From | Brassica oleracea |
| NO: 321 | sequence of | Transporter | | | var. oleracea |
| SEQ ID | DNA coding | Uptake | T10_BolGTR_GA | From | Brassica oleracea |
| NO: 322 | sequence of | Transporter | | | var. oleracea |
| SEQ ID | Amino acid | Uptake | T11_AthGTR1_GA | From | Arabidopsis |
| NO: 323 | sequence of | Transporter | | | thaliana |
| SEQ ID | DNA coding | Uptake | T11_AthGTR1_GA | From | Arabidopsis |
| NO: 324 | sequence of | Transporter | | | thaliana |
| SEQ ID | Amino acid | Uptake | T12_PsoNPF1_GA | From | Papaver |
| NO: 325 | sequence of | Transporter | | | somniferum |
| SEQ ID | DNA coding | Uptake | T12_PsoNPF1_GA | From | Papaver |
| NO: 326 | sequence of | Transporter | | | somniferum |
| SEQ ID | Amino acid | Uptake | T14_PsoNPF3_GA | From | Papaver |
| NO: 327 | sequence of | Transporter | | | somniferum |
| SEQ ID | DNA coding | Uptake | T14_PsoNPF3_GA | From | Papaver |
| NO: 328 | sequence of | Transporter | | | somniferum |
| SEQ ID | Amino acid | Uptake | T15_PsoNPF4_GA | From | Papaver |
| NO: 329 | sequence of | Transporter | | | somniferum |
| SEQ ID | DNA coding | Uptake | T15_PsoNPF4_GA | From | Papaver |
| NO: 330 | sequence of | Transporter | | | somniferum |
| SEQ ID | Amino acid | Uptake | T17_PsoNPF6_GA | From | Papaver |
| NO: 331 | sequence of | Transporter | | | somniferum |
| SEQ ID | DNA coding | Uptake | T17_PsoNPF6_GA | From | Papaver |
| NO: 332 | sequence of | Transporter | | | somniferum |
| SEQ ID | Amino acid | Uptake | T18_PsoNPF7_GA | From | Papaver |
| NO: 333 | sequence of | Transporter | | | somniferum |
| SEQ ID | DNA coding | Uptake | T18_PsoNPF7_GA | From | Papaver |
| NO: 334 | sequence of | Transporter | | | somniferum |
| SEQ ID | Amino acid | Uptake | T19_RmiPTR2_GA | From | Rhizopus |
| NO: 335 | sequence of | Transporter | | | microsporus |
| SEQ ID | DNA coding | Uptake | T19_RmiPTR2_GA | From | Rhizopus |
| NO: 336 | sequence of | Transporter | | | microsporus |
| SEQ ID | Amino acid | Uptake | T20_RmiPTR2_v2_GA | From | Rhizopus |
| NO: 337 | sequence of | Transporter | | | microsporus |
| SEQ ID | DNA coding | Uptake | T20_RmiPTR2_v2_GA | From | Rhizopus |
| NO: 338 | sequence of | Transporter | | | microsporus |
| SEQ ID | Amino acid | Uptake | T21_RalPTR2_GA | From | Rozella allomycis |
| NO: 339 | sequence of | Transporter | | | |
| SEQ ID | DNA coding | Uptake | T21_RalPTR2_GA | From | Rozella allomycis |
| NO: 340 | sequence of | Transporter | | | |
| SEQ ID | Amino acid | Uptake | T22_CanPOT_GA | From | Catenaria |
| NO: 341 | sequence of | Transporter | | | anguillulae |
| SEQ ID | DNA coding | Uptake | T22_CanPOT_GA | From | Catenaria |
| NO: 342 | sequence of | Transporter | | | anguillulae |
| SEQ ID | Amino acid | Uptake | T23_ArePOT_GA | From | Absidia repens |
| NO: 343 | sequence of | Transporter | | | |
| SEQ ID | DNA coding | Uptake | T23_ArePOT_GA | From | Absidia repens |
| NO: 344 | sequence of | Transporter | | | |
| SEQ ID | Amino acid | Uptake | T24_SlyPTR2_GA | From | Stemphylium |
| NO: 345 | sequence of | Transporter | | | lycopersici |
| SEQ ID | DNA coding | Uptake | T24_SlyPTR2_GA | From | Stemphylium |
| NO: 346 | sequence of | Transporter | | | lycopersici |
| SEQ ID | Amino acid | Uptake | T25_AorPOT_GA | From | Aspergillus oryzae |
| NO: 347 | sequence of | Transporter | | | |
| SEQ ID | DNA coding | Uptake | T25_AorPOT_GA | From | Aspergillus oryzae |
| NO: 348 | sequence of | Transporter | | | |
| SEQ ID | Amino acid | Uptake | T26_NfuPOT_GA | From | Neosartorya |
| NO: 349 | sequence of | Transporter | | | fumigata |
| SEQ ID | DNA coding | Uptake | T26_NfuPOT_GA | From | Neosartorya |
| NO: 350 | sequence of | Transporter | | | fumigata |
| SEQ ID | Amino acid | Uptake | T27_FoxPOT_GA | From | Fusarium |
| NO: 351 | sequence of | Transporter | | | oxysporum |
| SEQ ID | DNA coding | Uptake | T27_FoxPOT_GA | From | Fusarium |
| NO: 352 | sequence of | Transporter | | | oxysporum |
| SEQ ID | Amino acid | Uptake | T28_MciPOT_GA | From | Mucor circinelloides |
| NO: 353 | sequence of | Transporter | | | f. circinelloides |
| SEQ ID | DNA coding | Uptake | T28_MciPOT_GA | From | Mucor circinelloides |
| NO: 354 | sequence of | Transporter | | | f. circinelloides |
| SEQ ID | Amino acid | Uptake | T29_AcaPOT_GA | From | Aspergillus |
| NO: 355 | sequence of | Transporter | | | calidoustus |
| SEQ ID | DNA coding | Uptake | T29_AcaPOT_GA | From | Aspergillus |
| NO: 356 | sequence of | Transporter | | | calidoustus |
| SEQ ID | Amino acid | Uptake | T30_MlyPOT_GA | From | Microbotryum |
| NO: 357 | sequence of | Transporter | | | lychnidis-dioicae |
| SEQ ID | DNA coding | Uptake | T30_MlyPOT_GA | From | Microbotryum |
| NO: 358 | sequence of | Transporter | | | lychnidis-dioicae |
| SEQ ID | Amino acid | Uptake | T31_TgaPOT_GA | From | Trichoderma gamsii |
| NO: 359 | sequence of | Transporter | | | |
| SEQ ID | DNA coding | Uptake | T31_TgaPOT_GA | From | Trichoderma gamsii |
| NO: 360 | sequence of | Transporter | | | |
| SEQ ID | Amino acid | Uptake | T32_AarPOT_GA | From | Aspergillus |
| NO: 361 | sequence of | Transporter | | | arachidicola |
| SEQ ID | DNA coding | Uptake | T32_AarPOT_GA | From | Aspergillus |
| NO: 362 | sequence of | Transporter | | | arachidicola |
| SEQ ID | Amino acid | Uptake | T33_CcuPTR2_GA | From | Choanephora |
| NO: 363 | sequence of | Transporter | | | cucurbitarum |
| SEQ ID | DNA coding | Uptake | T33_CcuPTR2_GA | From | Choanephora |
| NO: 364 | sequence of | Transporter | | | cucurbitarum |
| SEQ ID | Amino acid | Uptake | T34_HvePOT_GA | From | Hesseltinella |
| NO: 365 | sequence of | Transporter | | | vesiculosa |
| SEQ ID | DNA coding | Uptake | T34_HvePOT_GA | From | Hesseltinella |
| NO: 366 | sequence of | Transporter | | | vesiculosa |
| SEQ ID | Amino acid | Uptake | T35_EcuPOT_GA | From | Encephalitozoon |
| NO: 367 | sequence of | Transporter | | | cuniculi |
| SEQ ID | DNA coding | Uptake | T35_EcuPOT_GA | From | Encephalitozoon |
| NO: 368 | sequence of | Transporter | | | cuniculi |
| SEQ ID | Amino acid | Uptake | T36_RnePOT_GA | From | Rosellinia necatrix |
| NO: 369 | sequence of | Transporter | | | |
| SEQ ID | DNA coding | Uptake | T36_RnePOT_GA | From | Rosellinia necatrix |
| NO: 370 | sequence of | Transporter | | | |
| SEQ ID | Amino acid | Uptake | T37_OcoPOT_GA | From | Ordospora colligata |
| NO: 371 | sequence of | Transporter | | | |
| SEQ ID | DNA coding | Uptake | T37_OcoPOT_GA | From | Ordospora colligata |
| NO: 372 | sequence of | Transporter | | | |
| SEQ ID | Amino acid | Uptake | T38_ScuPTR2_GA | From | Smittium culicis |
| NO: 373 | sequence of | Transporter | | | |
| SEQ ID | DNA coding | Uptake | T38_ScuPTR2_GA | From | Smittium culicis |
| NO: 374 | sequence of | Transporter | | | |
| SEQ ID | Amino acid | Uptake | T39_CgrPOT_GA | From | Colletotrichum |
| NO: 375 | sequence of | Transporter | | | graminicola |
| SEQ ID | DNA coding | Uptake | T39_CgrPOT_GA | From | Colletotrichum |
| NO: 376 | sequence of | Transporter | | | graminicola |
| SEQ ID | Amino acid | Uptake | T40_EdePOT_GA | From | Exophiala |
| NO: 377 | sequence of | Transporter | | | dermatitidis |
| SEQ ID | DNA coding | Uptake | T40_EdePOT_GA | From | Exophiala |
| NO: 378 | sequence of | Transporter | | | dermatitidis |
| SEQ ID | Amino acid | Uptake | T41_CalPTR2_GA | From | Candida albicans |
| NO: 379 | sequence of | Transporter | | | |
| SEQ ID | DNA coding | Uptake | T41_CalPTR2_GA | From | Candida albicans |
| NO: 380 | sequence of | Transporter | | | |
| SEQ ID | Amino acid | Uptake | T44_CcaMFS_GA | From | Cajanus cajan |
| NO: 381 | sequence of | Transporter | | | |
| SEQ ID | DNA coding | Uptake | T44_CcaMFS_GA | From | Cajanus cajan |
| NO: 382 | sequence of | Transporter | | | |
| SEQ ID | Amino acid | Uptake | T45_PanPOT_GA | From | Parasponia |
| NO: 383 | sequence of | Transporter | | | andersonii |
| SEQ ID | DNA coding | Uptake | T45_PanPOT_GA | From | Parasponia |
| NO: 384 | sequence of | Transporter | | | andersonii |
| SEQ ID | Amino acid | Uptake | T46_RchPOT_GA | From | Rosa chinensis |
| NO: 385 | sequence of | Transporter | | | |
| SEQ ID | DNA coding | Uptake | T46_RchPOT_GA | From | Rosa chinensis |
| NO: 386 | sequence of | Transporter | | | |
| SEQ ID | Amino acid | Uptake | T47_PbeNPF_GA | From | Pyrus betulifolia |
| NO: 387 | sequence of | Transporter | | | |
| SEQ ID | DNA coding | Uptake | T47_PbeNPF_GA | From | Pyrus betulifolia |
| NO: 388 | sequence of | Transporter | | | |
| SEQ ID | Amino acid | Uptake | T48_CcaPOT_GA | From | Corchorus |
| NO: 389 | sequence of | Transporter | | | capsularis |
| SEQ ID | DNA coding | Uptake | T48_CcaPOT_GA | From | Corchorus |
| NO: 390 | sequence of | Transporter | | | capsularis |
| SEQ ID | Amino acid | Uptake | T49_HanPOT_GA | From | Helianthus annuus |
| NO: 391 | sequence of | Transporter | | | |
| SEQ ID | DNA coding | Uptake | T49_HanPOT_GA | From | Helianthus annuus |
| NO: 392 | sequence of | Transporter | | | |
| SEQ ID | Amino acid | Uptake | T50_HimPOT_GA | From | Handroanthus |
| NO: 393 | sequence of | Transporter | | | impetiginosus |
| SEQ ID | DNA coding | Uptake | T50_HimPOT_GA | From | Handroanthus |
| NO: 394 | sequence of | Transporter | | | impetiginosus |
| SEQ ID | Amino acid | Uptake | T51_TorPOT_GA | From | Trema orientalis |
| NO: 395 | sequence of | Transporter | | | |
| SEQ ID | DNA coding | Uptake | T51_TorPOT_GA | From | Trema orientalis |
| NO: 396 | sequence of | Transporter | | | |
| SEQ ID | Amino acid | Uptake | T52_BmePTR2_GA | From | Basidiobolus |
| NO: 397 | sequence of | Transporter | | | meristosporus |
| SEQ ID | DNA coding | Uptake | T52_BmePTR2_GA | From | Basidiobolus |
| NO: 398 | sequence of | Transporter | | | meristosporus |
| SEQ ID | Amino acid | Uptake | T53_EhePOT_GA | From | Encephalitozoon |
| NO: 399 | sequence of | Transporter | | | hellem |
| SEQ ID | DNA coding | Uptake | T53_EhePOT_GA | From | Encephalitozoon |
| NO: 400 | sequence of | Transporter | | | hellem |
| SEQ ID | Amino acid | Uptake | T54_MelPOT_GA | From | Mortierella elongata |
| NO: 401 | sequence of | Transporter | | | |
| SEQ ID | DNA coding | Uptake | T54_MelPOT_GA | From | Mortierella elongata |
| NO: 402 | sequence of | Transporter | | | |
| SEQ ID | Amino acid | Uptake | T55_NsyNPF_GA | From | Nicotiana sylvestris |
| NO: 403 | sequence of | Transporter | | | |
| SEQ ID | DNA coding | Uptake | T55_NsyNPF_GA | From | Nicotiana sylvestris |
| NO: 404 | sequence of | Transporter | | | |
| SEQ ID | Amino acid | Uptake | T56_CanNPF_GA | From | Capsicum annuum |
| NO: 405 | sequence of | Transporter | | | |
| SEQ ID | DNA coding | Uptake | T56_CanNPF_GA | From | Capsicum annuum |
| NO: 406 | sequence of | Transporter | | | |
| SEQ ID | Amino acid | Uptake | T57_AcoNPF_GA | From | Aquilegia coerulea |
| NO: 407 | sequence of | Transporter | | | |
| SEQ ID | DNA coding | Uptake | T57_AcoNPF_GA | From | Aquilegia coerulea |
| NO: 408 | sequence of | Transporter | | | |
| SEQ ID | Amino acid | Uptake | T59_AmeNPF1_GA | From | Argemone mexican |
| NO: 409 | sequence of | Transporter | | | |
| SEQ ID | DNA coding | Uptake | T59_AmeNPF1_GA | From | Argemone mexican |
| NO: 410 | sequence of | Transporter | | | |
| SEQ ID | Amino acid | Uptake | T60_AmeNPF2_GA | From | Argemone mexican |
| NO: 411 | sequence of | Transporter | | | |
| SEQ ID | DNA coding | Uptake | T60_AmeNPF2_GA | From | Argemone mexican |
| NO: 412 | sequence of | Transporter | | | |
| SEQ ID | Amino acid | Uptake | T61_TwiNPF_GA | From | Tripterygium |
| NO: 413 | sequence of | Transporter | | | wilfordii |
| SEQ ID | DNA coding | Uptake | T61_TwiNPF_GA | From | Tripterygium |
| NO: 414 | sequence of | Transporter | | | wilfordii |
| SEQ ID | Amino acid | Uptake | T62_SmaNPF_GA | From | Swietenia |
| NO: 415 | sequence of | Transporter | | | mahagoni |
| SEQ ID | DNA coding | Uptake | T62_SmaNPF_GA | From | Swietenia |
| NO: 416 | sequence of | Transporter | | | mahagoni |
| SEQ ID | Amino acid | Uptake | T63_CfoNPF_GA | From | Coleus forskohlii |
| NO: 417 | sequence of | Transporter | | | |
| SEQ ID | DNA coding | Uptake | T63_CfoNPF_GA | From | Coleus forskohlii |
| NO: 418 | sequence of | Transporter | | | |
| SEQ ID | Amino acid | Uptake | T64_XsiNPF_GA | From | Xanthorhiza |
| NO: 419 | sequence of | Transporter | | | simplicissima |
| SEQ ID | DNA coding | Uptake | T64_XsiNPF_GA | From | Xanthorhiza |
| NO: 420 | sequence of | Transporter | | | simplicissima |
| SEQ ID | Amino acid | Uptake | T66_TeINPF_GA | From | Tabernaemontana |
| NO: 421 | sequence of | Transporter | | | elegans |
| SEQ ID | DNA coding | Uptake | T66_TeINPF_GA | From | Tabernaemontana |
| NO: 422 | sequence of | Transporter | | | elegans |
| SEQ ID | Amino acid | Uptake | T67_SdiNPF_GA | From | Stylophorum |
| NO: 423 | sequence of | Transporter | | | diphyllum |
| SEQ ID | DNA coding | Uptake | T67_SdiNPF_GA | From | Stylophorum |
| NO: 424 | sequence of | Transporter | | | diphyllum |
| SEQ ID | Amino acid | Uptake | T68_RseNPF_GA | From | Rauwolfia |
| NO: 425 | sequence of | Transporter | | | serpentina |
| SEQ ID | DNA coding | Uptake | T68_RseNPF_GA | From | Rauwolfia |
| NO: 426 | sequence of | Transporter | | | serpentina |
| SEQ ID | Amino acid | Uptake | T69_PhoNPF_GA | From | pelargonium × |
| NO: 427 | sequence of | Transporter | | | hortorum |
| SEQ ID | DNA coding | Uptake | T69_PhoNPF_GA | From | pelargonium × |
| NO: 428 | sequence of | Transporter | | | hortorum |
| SEQ ID | Amino acid | Uptake | T70_CmaNPF_GA | From | Chelidonium majus |
| NO: 429 | sequence of | Transporter | | | |
| SEQ ID | DNA coding | Uptake | T70_CmaNPF_GA | From | Chelidonium majus |
| NO: 430 | sequence of | Transporter | | | |
| SEQ ID | Amino acid | Uptake | T71_CchNPF_GA | From | Corydalis |
| NO: 431 | sequence of | Transporter | | | chelanthifolia |
| SEQ ID | DNA coding | Uptake | T71_CchNPF_GA | From | Corydalis |
| NO: 432 | sequence of | Transporter | | | chelanthifolia |
| SEQ ID | Amino acid | Uptake | T72_TcoNPF_GA | From | Tinospora_cordifolia |
| NO: 433 | sequence of | Transporter | | | |
| SEQ ID | DNA coding | Uptake | T72_TcoNPF_GA | From | Tinospora_cordifolia |
| NO: 434 | sequence of | Transporter | | | |
| SEQ ID | Amino acid | Uptake | T73_PbrNPF1_GA | From | Papaver |
| NO: 435 | sequence of | Transporter | | | bracteatum |
| SEQ ID | DNA coding | Uptake | T73_PbrNPF1_GA | From | Papaver |
| NO: 436 | sequence of | Transporter | | | bracteatum |
| SEQ ID | Amino acid | Uptake | T74_PbrNPF2_GA | From | Papaver |
| NO: 437 | sequence of | Transporter | | | bracteatum |
| SEQ ID | DNA coding | Uptake | T74_PbrNPF2_GA | From | Papaver |
| NO: 438 | sequence of | Transporter | | | bracteatum |
| SEQ ID | Amino acid | Uptake | T75_PbrNPF3_GA | From | Papaver |
| NO: 439 | sequence of | Transporter | | | bracteatum |
| SEQ ID | DNA coding | Uptake | T75_PbrNPF3_GA | From | Papaver |
| NO: 440 | sequence of | Transporter | | | bracteatum |
| SEQ ID | Amino acid | Uptake | T76_AhuNPF_GA | From | Amsonia hubrichtii |
| NO: 441 | sequence of | Transporter | | | |
| SEQ ID | DNA coding | Uptake | T76_AhuNPF_GA | From | Amsonia hubrichtii |
| NO: 442 | sequence of | Transporter | | | |
| SEQ ID | Amino acid | Uptake | T77_PocNPF_GA | From | Platanus |
| NO: 443 | sequence of | Transporter | | | occidentalis |
| SEQ ID | DNA coding | Uptake | T77_PocNPF_GA | From | Platanus |
| NO: 444 | sequence of | Transporter | | | occidentalis |
| SEQ ID | Amino acid | Uptake | T78_VofNPF_GA | From | Valeriana officinalis |
| NO: 445 | sequence of | Transporter | | | |
| SEQ ID | DNA coding | Uptake | T78_VofNPF_GA | From | Valeriana officinalis |
| NO: 446 | sequence of | Transporter | | | |
| SEQ ID | Amino acid | Uptake | T79_EcaNPF_GA | From | Eschscholzia |
| NO: 447 | sequence of | Transporter | | | californica |
| SEQ ID | DNA coding | Uptake | T79_EcaNPF_GA | From | Eschscholzia |
| NO: 448 | sequence of | Transporter | | | californica |
| SEQ ID | Amino acid | Uptake | T80_CroNPF_GA | From | Catharanthus |
| NO: 449 | sequence of | Transporter | | | roseus |
| SEQ ID | DNA coding | Uptake | T80_CroNPF_GA | From | Catharanthus |
| NO: 450 | sequence of | Transporter | | | roseus |
| SEQ ID | Amino acid | Uptake | T81_HcaNPF_GA | From | Hypericum |
| NO: 451 | sequence of | Transporter | | | perforatum |
| SEQ ID | DNA coding | Uptake | T81_HcaNPF_GA | From | Hypericum |
| NO: 452 | sequence of | Transporter | | | perforatum |
| SEQ ID | Amino acid | Uptake | T82_NsaNPF_GA | From | Nigella sativa |
| NO: 453 | sequence of | Transporter | | | |
| SEQ ID | DNA coding | Uptake | T82_NsaNPF_GA | From | Nigella sativa |
| NO: 454 | sequence of | Transporter | | | |
| SEQ ID | Amino acid | Uptake | T83_ScaNPF_GA | From | Sanguinaria |
| NO: 455 | sequence of | Transporter | | | canadensis |
| SEQ ID | DNA coding | Uptake | T83_ScaNPF_GA | From | Sanguinaria |
| NO: 456 | sequence of | Transporter | | | canadensis |
| SEQ ID | Amino acid | Uptake | T84_TflNPF_GA | From | Thalictrum flavum |
| NO: 457 | sequence of | Transporter | | | |
| SEQ ID | DNA coding | Uptake | T84_TflNPF_GA | From | Thalictrum flavum |
| NO: 458 | sequence of | Transporter | | | |
| SEQ ID | Amino acid | Uptake | T85_GflNPF_GA | From | Glaucium Flavum |
| NO: 459 | sequence of | Transporter | | | |
| SEQ ID | DNA coding | Uptake | T85_GflNPF_GA | From | Glaucium Flavum |
| NO: 460 | sequence of | Transporter | | | |
| SEQ ID | Amino acid | Uptake | T97_ScaT14_GA | From | Sanguinaria |
| NO: 461 | sequence of | Transporter | | | canadensis |
| SEQ ID | DNA coding | Uptake | T97_ScaT14_GA | From | Sanguinaria |
| NO: 462 | sequence of | Transporter | | | canadensis |
| SEQ ID | Amino acid | Uptake | T101_McoPUP3_1 | From | Macleaya cordata |
| NO: 463 | sequence of | Transporter | | | |
| SEQ ID | DNA coding | Uptake | T101_McoPUP3_1 | From | Macleaya cordata |
| NO: 464 | sequence of | Transporter | | | |
| SEQ ID | Amino acid | Uptake | T102_PsoPUP3_1 | From | Papaver |
| NO: 465 | sequence of | Transporter | | | somniferum |
| SEQ ID | DNA coding | Uptake | T102_PsoPUP3_1 | From | Papaver |
| NO: 466 | sequence of | Transporter | | | somniferum |
| SEQ ID | Amino acid | Uptake | T103_PsoPUP3_2 | From | Papaver |
| NO: 467 | sequence of | Transporter | | | somniferum |
| SEQ ID | DNA coding | Uptake | T103_PsoPUP3_2 | From | Papaver |
| NO: 468 | sequence of | Transporter | | | somniferum |
| SEQ ID | Amino acid | Uptake | T104_PsoPUP3_3 | From | Papaver |
| NO: 469 | sequence of | Transporter | | | somniferum |
| SEQ ID | DNA coding | Uptake | T104_PsoPUP3_3 | From | Papaver |
| NO: 470 | sequence of | Transporter | | | somniferum |
| SEQ ID | Amino acid | Uptake | T105_PsoPUP-L | From | Papaver |
| NO: 471 | sequence of | Transporter | | | somniferum |
| SEQ ID | DNA coding | Uptake | T105_PsoPUP-L | From | Papaver |
| NO: 472 | sequence of | Transporter | | | somniferum |
| SEQ ID | Amino acid | Uptake | T109_GflPUP3_83 | From | Glaucium Flavum |
| NO: 473 | sequence of | Transporter | | | |
| SEQ ID | DNA coding | Uptake | T109_GflPUP3_83 | From | Glaucium Flavum |
| NO: 474 | sequence of | Transporter | | | |
| SEQ ID | Amino acid | Uptake | T113_PsoPUP3_32 | From | Papaver |
| NO: 475 | sequence of | Transporter | | | somniferum |
| SEQ ID | DNA coding | Uptake | T113_PsoPUP3_32 | From | Papaver |
| NO: 476 | sequence of | Transporter | | | somniferum |
| SEQ ID | Amino acid | Uptake | T114_TorPUP3_40 | From | Trema orientale |
| NO: 477 | sequence of | Transporter | | | |
| SEQ ID | DNA coding | Transporter | T114_TorPUP3_40 | From | Trema orientale |
| NO: 478 | sequence of | | | | |
| SEQ ID | Amino acid | Uptake | T115_CsaPUP3_48 | From | Cucumis sativus |
| NO: 479 | sequence of | Transporter | | | |
| SEQ ID | DNA coding | Uptake | T115_CsaPUP3_48 | From | Cucumis sativus |
| NO: 480 | sequence of | Transporter | | | |
| SEQ ID | Amino acid | Uptake | T116_HanPUP3_56 | From | Helianthus annuus |
| NO: 481 | sequence of | Transporter | | | |
| SEQ ID | DNA coding | Uptake | T116_HanPUP3_56 | From | Helianthus annuus |
| NO: 482 | sequence of | Transporter | | | |
| SEQ ID | Amino acid | Uptake | T117_MacPUP3_64 | From | Musa acuminata |
| NO: 483 | sequence of | Transporter | | | |
| SEQ ID | DNA coding | Uptake | T117_MacPUP3_64 | From | Musa acuminata |
| NO: 484 | sequence of | Transporter | | | |
| SEQ ID | Amino acid | Uptake | T121_NnuPUP3_9 | From | Nelumbo nucifera |
| NO: 485 | sequence of | Transporter | | | |
| SEQ ID | DNA coding | Uptake | T121_NnuPUP3_9 | From | Nelumbo nucifera |
| NO: 486 | sequence of | Transporter | | | |
| SEQ ID | Amino acid | Uptake | T122_PsoPUP3_17 | From | Papaver |
| NO: 487 | sequence of | Transporter | | | somniferum |
| SEQ ID | DNA coding | Uptake | T122_PsoPUP3_17 | From | Papaver |
| NO: 488 | sequence of | Transporter | | | somniferum |
| SEQ ID | Amino acid | Uptake | T123_PsoPUP3_25 | From | Papaver |
| NO: 489 | sequence of | Transporter | | | somniferum |
| SEQ ID | DNA coding | Uptake | T123_PsoPUP3_25 | From | Papaver |
| NO: 490 | sequence of | Transporter | | | somniferum |
| SEQ ID | Amino acid | Uptake | T124_PsoPUP3_33 | From | Papaver |
| NO: 491 | sequence of | Transporter | | | somniferum |
| SEQ ID | DNA coding | Uptake | T124_PsoPUP3_33 | From | Papaver |
| NO: 492 | sequence of | Transporter | | | somniferum |
| SEQ ID | Amino acid | Uptake | T125_JcuPUP3_41 | From | Jatropha curcas |
| NO: 493 | sequence of | Transporter | | | |
| SEQ ID | DNA coding | Uptake | T125_JcuPUP3_41 | From | Jatropha curcas |
| NO: 494 | sequence of | Transporter | | | |
| SEQ ID | Amino acid | Uptake | T126_CpePUP3_49 | From | Cucurbita pepo |
| NO: 495 | sequence of | Transporter | | | subsp. pepo |
| SEQ ID | DNA coding | Uptake | T126_CpePUP3_49 | From | Cucurbita pepo |
| NO: 496 | sequence of | Transporter | | | subsp. pepo |
| SEQ ID | Amino acid | Uptake | T127_LsaPUP3_57 | From | Lactuca sativa |
| NO: 497 | sequence of | Transporter | | | |
| SEQ ID | DNA coding | Uptake | T127_LsaPUP3_57 | From | Lactuca sativa |
| NO: 498 | sequence of | Transporter | | | |
| SEQ ID | Amino acid | Uptake | T128_PsoPUP3_65 | From | Papaver |
| NO: 499 | sequence of | Transporter | | | somniferum |
| SEQ ID | DNA coding | Uptake | T128_PsoPUP3_65 | From | Papaver |
| NO: 500 | sequence of | Transporter | | | somniferum |
| SEQ ID | Amino acid | Uptake | T129_PsoPUP3_73 | From | Papaver |
| NO: 501 | sequence of | Transporter | | | somniferum |
| SEQ ID | DNA coding | Uptake | T129_PsoPUP3_73 | From | Papaver |
| NO: 502 | sequence of | Transporter | | | somniferum |
| SEQ ID | Amino acid | Uptake | T130_NdoPUP3_89 | From | Nandina domestica |
| NO: 503 | sequence of | Transporter | | | |
| SEQ ID | DNA coding | Uptake | T130_NdoPUP3_89 | From | Nandina domestica |
| NO: 504 | sequence of | Transporter | | | |
| SEQ ID | Amino acid | Uptake | T131_PbrPUP3_81 | From | Papaver |
| NO: 505 | sequence of | Transporter | | | bracteatum |
| SEQ ID | DNA coding | Uptake | T131_PbrPUP3_81 | From | Papaver |
| NO: 506 | sequence of | Transporter | | | bracteatum |
| SEQ ID | Amino acid | Uptake | T132_CmiPUP3_10 | From | Cinnamomum |
| NO: 507 | sequence of | Transporter | | | micranthum |
| | | | | | f. kanehirae |
| SEQ ID | DNA coding | Uptake | T132_CmiPUP3_10 | From | Cinnamomum |
| NO: 508 | sequence of | Transporter | | | micranthum |
| | | | | | f. kanehirae |
| SEQ ID | Amino acid | Uptake | T133_PsoPUP3_18 | From | Papaver |
| NO: 509 | sequence of | Transporter | | | somniferum |
| SEQ ID | DNA coding | Uptake | T133_PsoPUP3_18 | From | Papaver |
| NO: 510 | sequence of | Transporter | | | somniferum |
| SEQ ID | Amino acid | Uptake | T135_PsoPUP_34 | From | Papaver |
| NO: 511 | sequence of | Transporter | | | somniferum |
| SEQ ID | DNA coding | Uptake | T135_PsoPUP_34 | From | Papaver |
| NO: 512 | sequence of | Transporter | | | somniferum |
| SEQ ID | Amino acid | Uptake | T136_RchPUP3_42 | From | Rosa chinensis |
| NO: 513 | sequence of | Transporter | | | |
| SEQ ID | DNA coding | Uptake | T136_RchPUP3_42 | From | Rosa chinensis |
| NO: 514 | sequence of | Transporter | | | |
| SEQ ID | Amino acid | Uptake | T137_EguPUP3_50 | From | Erythranthe guttata |
| NO: 515 | sequence of | Transporter | | | |
| SEQ ID | DNA coding | Uptake | T137_EguPUP3_50 | From | Erythranthe guttata |
| NO: 516 | sequence of | Transporter | | | |
| SEQ ID | Amino acid | Uptake | T138_AduPUP3_58 | From | Arachis duranensis |
| NO: 517 | sequence of | Transporter | | | |
| SEQ ID | DNA coding | Uptake | T138_AduPUP3_58 | From | Arachis duranensis |
| NO: 518 | sequence of | Transporter | | | |
| SEQ ID | Amino acid | Uptake | T139_PsoPUP3_66 | From | Papaver |
| NO: 519 | sequence of | Transporter | | | somniferum |
| SEQ ID | DNA coding | Uptake | T139_PsoPUP3_66 | From | Papaver |
| NO: 520 | sequence of | Transporter | | | somniferum |
| SEQ ID | Amino acid | Uptake | T140_PalPUP3_74 | From | Papaver alpinum |
| NO: 521 | sequence of | Transporter | | | |
| SEQ ID | DNA coding | Uptake | T140_PalPUP3_74 | From | Papaver alpinum |
| NO: 522 | sequence of | Transporter | | | |
| SEQ ID | Amino acid | Uptake | T141_EcaPUP3_88 | From | Eschscholzia |
| NO: 523 | sequence of | Transporter | | | californica |
| SEQ ID | DNA coding | Uptake | T141_EcaPUP3_88 | From | Eschscholzia |
| NO: 524 | sequence of | Transporter | | | californica |
| SEQ ID | Amino acid | Uptake | T142_McoPUP3_4 | From | Macleaya cordata |
| NO: 525 | sequence of | Transporter | | | |
| SEQ ID | DNA coding | Uptake | T142_McoPUP3_4 | From | Macleaya cordata |
| NO: 526 | sequence of | Transporter | | | |
| SEQ ID | Amino acid | Uptake | T143_CmiPUP3_11 | From | Cinnamomum |
| NO: 527 | sequence of | Transporter | | | micranthum |
| | | | | | f. kanehirae |
| SEQ ID | DNA coding | Uptake | T143_CmiPUP3_11 | From | Cinnamomum |
| NO: 528 | sequence of | Transporter | | | micranthum |
| | | | | | f. kanehirae |
| SEQ ID | Amino acid | Uptake | T144_PsoPUP3_19 | From | Papaver |
| NO: 529 | sequence of | Transporter | | | somniferum |
| SEQ ID | DNA coding | Uptake | T144_PsoPUP3_19 | From | Papaver |
| NO: 530 | sequence of | Transporter | | | somniferum |
| SEQ ID | Amino acid | Uptake | T146_PsoPUP_35 | From | Papaver |
| NO: 531 | sequence of | Transporter | | | somniferum |
| SEQ ID | DNA coding | Uptake | T146_PsoPUP_35 | From | Papaver |
| NO: 532 | sequence of | Transporter | | | somniferum |
| SEQ ID | Amino acid | Uptake | T147_MesPUP3_43 | From | Manihot esculenta |
| NO: 533 | sequence of | Transporter | | | |
| SEQ ID | DNA coding | Uptake | T147_MesPUP3_43 | From | Manihot esculenta |
| NO: 534 | sequence of | Transporter | | | |
| SEQ ID | Amino acid | Uptake | T148_HimPUP3_51 | From | Handroanthus |
| NO: 535 | sequence of | Transporter | | | impetiginosus |
| SEQ ID | DNA coding | Uptake | T148_HimPUP3_51 | From | Handroanthus |
| NO: 536 | sequence of | Transporter | | | impetiginosus |
| SEQ ID | Amino acid | Uptake | T149_AcoPUP3_59 | From | Aquilegia coerulea |
| NO: 537 | sequence of | Transporter | | | |
| SEQ ID | DNA coding | Uptake | T149_AcoPUP3_59 | From | Aquilegia coerulea |
| NO: 538 | sequence of | Transporter | | | |
| SEQ ID | Amino acid | Uptake | T150_PsoPUP3_67 | From | Papaver |
| NO: 539 | sequence of | Transporter | | | somniferum |
| SEQ ID | DNA coding | Uptake | T150_PsoPUP3_67 | From | Papaver |
| NO: 540 | sequence of | Transporter | | | somniferum |
| SEQ ID | Amino acid | Uptake | T151_PatPUP3_75 | From | Papaver atlanticum |
| NO: 541 | sequence of | Transporter | | | |
| SEQ ID | DNA coding | Uptake | T151_PatPUP3_75 | From | Papaver atlanticum |
| NO: 542 | sequence of | Transporter | | | |
| SEQ ID | Amino acid | Uptake | T152_GflPUP3_87 | From | Glaucium Flavum |
| NO: 543 | sequence of | Transporter | | | |
| SEQ ID | DNA coding | Uptake | T152_GflPUP3_87 | From | Glaucium Flavum |
| NO: 544 | sequence of | Transporter | | | |
| SEQ ID | Amino acid | Uptake | T153_PsoPUP3_5 | From | Papaver |
| NO: 545 | sequence of | Transporter | | | somniferum |
| SEQ ID | DNA coding | Uptake | T153_PsoPUP3_5 | From | Papaver |
| NO: 546 | sequence of | Transporter | | | somniferum |
| SEQ ID | Amino acid | Uptake | T154_CmiPUP3_12 | From | Cinnamomum |
| NO: 547 | sequence of | Transporter | | | micranthum |
| | | | | | f. kanehirae |
| SEQ ID | DNA coding | Uptake | T154_CmiPUP3_12 | From | Cinnamomum |
| NO: 548 | sequence of | Transporter | | | micranthum |
| | | | | | f. kanehirae |
| SEQ ID | Amino acid | Uptake | T156_PsoPUP3_28 | From | Papaver |
| NO: 549 | sequence of | Transporter | | | somniferum |
| SEQ ID | DNA coding | Uptake | T156_PsoPUP3_28 | From | Papaver |
| NO: 550 | sequence of | Transporter | | | somniferum |
| SEQ ID | Amino acid | Uptake | T157_RchPUP_36 | From | Rosa chinensis |
| NO: 551 | sequence of | Transporter | | | |
| SEQ ID | DNA coding | Uptake | T157_RchPUP_36 | From | Rosa chinensis |
| NO: 552 | sequence of | Transporter | | | |
| SEQ ID | Amino acid | Uptake | T158_DziPUP3_44 | From | Durio zibethinus |
| NO: 553 | sequence of | Transporter | | | |
| SEQ ID | DNA coding | Uptake | T158_DziPUP3_44 | From | Durio zibethinus |
| NO: 554 | sequence of | Transporter | | | |
| SEQ ID | Amino acid | Uptake | T159_OeuPUP3_52 | From | Olea europaea var. |
| NO: 555 | sequence of | Transporter | | | sylvestris |
| SEQ ID | DNA coding | Uptake | T159_OeuPUP3_52 | From | Olea europaea var. |
| NO: 556 | sequence of | Transporter | | | sylvestris |
| SEQ ID | Amino acid | Uptake | T160_CeuPUP3_60 | From | Coffea eugenioides |
| NO: 557 | sequence of | Transporter | | | |
| SEQ ID | DNA coding | Uptake | T160_CeuPUP3_60 | From | Coffea eugenioides |
| NO: 558 | sequence of | Transporter | | | |
| SEQ ID | Amino acid | Uptake | T161_PsoPUP3_68 | From | Papaver |
| NO: 559 | sequence of | Transporter | | | somniferum |
| SEQ ID | DNA coding | Uptake | T161_PsoPUP3_68 | From | Papaver |
| NO: 560 | sequence of | Transporter | | | somniferum |
| SEQ ID | Amino acid | Uptake | T162_PmiPUP3_76 | From | Papaver |
| NO: 561 | sequence of | Transporter | | | miyabeanum |
| SEQ ID | DNA coding | Uptake | T162_PmiPUP3_76 | From | Papaver |
| NO: 562 | sequence of | Transporter | | | miyabeanum |
| SEQ ID | Amino acid | Uptake | T163_PbrPUP3_86 | From | Papaver |
| NO: 563 | sequence of | Transporter | | | bracteatum |
| SEQ ID | DNA coding | Uptake | T163_PbrPUP3_86 | From | Papaver |
| NO: 564 | sequence of | Transporter | | | bracteatum |
| SEQ ID | Amino acid | Uptake | T164_PsoPUP3_78 | From | Papaver |
| NO: 565 | sequence of | Transporter | | | somniferum |
| SEQ ID | DNA coding | Uptake | T164_PsoPUP3_78 | From | Papaver |
| NO: 566 | sequence of | Transporter | | | somniferum |
| SEQ ID | Amino acid | Uptake | T165_AcoPUP3_13 | From | Aquilegia coerulea |
| NO: 567 | sequence of | Transporter | | | |
| SEQ ID | DNA coding | Uptake | T165_AcoPUP3_13 | From | Aquilegia coerulea |
| NO: 568 | sequence of | Transporter | | | |
| SEQ ID | Amino acid | Uptake | T166_PsoPUP3_21 | From | Papaver |
| NO: 569 | sequence of | Transporter | | | somniferum |
| SEQ ID | DNA coding | Uptake | T166_PsoPUP3_21 | From | Papaver |
| NO: 570 | sequence of | Transporter | | | somniferum |
| SEQ ID | Amino acid | Uptake | T168_FvePUP3_37 | From | Fragaria vesca |
| NO: 571 | sequence of | Transporter | | | subsp. vesca |
| SEQ ID | DNA coding | Uptake | T168_FvePUP3_37 | From | Fragaria vesca |
| NO: 572 | sequence of | Transporter | | | subsp. vesca |
| SEQ ID | Amino acid | Uptake | T169_ZjuPUP3_45 | From | Ziziphus jujuba |
| NO: 573 | sequence of | Transporter | | | |
| SEQ ID | DNA coding | Uptake | T169_ZjuPUP3_45 | From | Ziziphus jujuba |
| NO: 574 | sequence of | Transporter | | | |
| SEQ ID | Amino acid | Uptake | T170_LsaPUP3_53 | From | Lactuca sativa |
| NO: 575 | sequence of | Transporter | | | |
| SEQ ID | DNA coding | Uptake | T170_LsaPUP3_53 | From | Lactuca sativa |
| NO: 576 | sequence of | Transporter | | | |
| SEQ ID | Amino acid | Uptake | T171_McoPUP3_61 | From | Macleaya cordata |
| NO: 577 | sequence of | Transporter | | | |
| SEQ ID | DNA coding | Uptake | T171_McoPUP3_61 | From | Macleaya cordata |
| NO: 578 | sequence of | Transporter | | | |
| SEQ ID | Amino acid | Uptake | T172_AcoPUP3_69 | From | Aquilegia coerulea |
| NO: 579 | sequence of | Transporter | | | |
| SEQ ID | DNA coding | Uptake | T172_AcoPUP3_69 | From | Aquilegia coerulea |
| NO: 580 | sequence of | Transporter | | | |
| SEQ ID | Amino acid | Uptake | T173_PnuPUP3_77 | From | Papaver nudicale |
| NO: 581 | sequence of | Transporter | | | |
| SEQ ID | DNA coding | Uptake | T173_PnuPUP3_77 | From | Papaver nudicale |
| NO: 582 | sequence of | Transporter | | | |
| SEQ ID | Amino acid | Uptake | T174_PbrPUP3_85 | From | Papaver |
| NO: 583 | sequence of | Transporter | | | bracteatum |
| SEQ ID | DNA coding | Uptake | T174_PbrPUP3_85 | From | Papaver |
| NO: 584 | sequence of | Transporter | | | bracteatum |
| SEQ ID | Amino acid | Uptake | T175_PsoPUP3_6 | From | Papaver |
| NO: 585 | sequence of | Transporter | | | somniferum |
| SEQ ID | DNA coding | Uptake | T175_PsoPUP3_6 | From | Papaver |
| NO: 586 | sequence of | Transporter | | | somniferum |
| SEQ ID | Amino acid | Uptake | T176_AcoPUP3_14 | From | Aquilegia coerulea |
| NO: 587 | sequence of | Transporter | | | |
| SEQ ID | DNA coding | Uptake | T176_AcoPUP3_14 | From | Aquilegia coerulea |
| NO: 588 | sequence of | Transporter | | | |
| SEQ ID | Amino acid | Uptake | T177_PsoPUP3_22 | From | Papaver |
| NO: 589 | sequence of | Transporter | | | somniferum |
| SEQ ID | DNA coding | Uptake | T177_PsoPUP3_22 | From | Papaver |
| NO: 590 | sequence of | Transporter | | | somniferum |
| SEQ ID | Amino acid | Uptake | T178_PsoPUP3_30 | From | Papaver |
| NO: 591 | sequence of | Transporter | | | somniferum |
| SEQ ID | DNA coding | Uptake | T178_PsoPUP3_30 | From | Papaver |
| NO: 592 | sequence of | Transporter | | | somniferum |
| SEQ ID | Amino acid | Uptake | T179_PyePUP3_38 | From | Prunus yedoensis |
| NO: 593 | sequence of | Transporter | | | var. nudiflora |
| SEQ ID | DNA coding | Uptake | T179_PyePUP3_38 | From | Prunus yedoensis |
| NO: 594 | sequence of | Transporter | | | var. nudiflora |
| SEQ ID | Amino acid | Uptake | T180_McoPUP3_46 | From | Macleaya cordata |
| NO: 595 | sequence of | Transporter | | | |
| SEQ ID | DNA coding | Uptake | T180_McoPUP3_46 | From | Macleaya cordata |
| NO: 596 | sequence of | Transporter | | | |
| SEQ ID | Amino acid | Uptake | T181_HanPUP3_54 | From | Helianthus annuus |
| NO: 597 | sequence of | Transporter | | | |
| SEQ ID | DNA coding | Uptake | T181_HanPUP3_54 | From | Helianthus annuus |
| NO: 598 | sequence of | Transporter | | | |
| SEQ ID | Amino acid | Uptake | T182_CpaPUP3_62 | From | Carica papaya |
| NO: 599 | sequence of | Transporter | | | |
| SEQ ID | DNA coding | Uptake | T182_CpaPUP3_62 | From | Carica papaya |
| NO: 600 | sequence of | Transporter | | | |
| SEQ ID | Amino acid | Uptake | T184_PraPUP3_79 | From | Papaver radicatum |
| NO: 601 | sequence of | Transporter | | | |
| SEQ ID | DNA coding | Uptake | T184_PraPUP3_79 | From | Papaver radicatum |
| NO: 602 | sequence of | Transporter | | | |
| SEQ ID | Amino acid | Uptake | T186_ScaPUP3_84 | From | Sanguinaria |
| NO: 603 | sequence of | Transporter | | | canadensis |
| SEQ ID | DNA coding | Uptake | T186_ScaPUP3_84 | From | Sanguinaria |
| NO: 604 | sequence of | Transporter | | | canadensis |
| SEQ ID | Amino acid | Uptake | T188_AcoPUP3_15 | From | Aquilegia coerulea |
| NO: 605 | sequence of | Transporter | | | |
| SEQ ID | DNA coding | Uptake | T188_AcoPUP3_15 | From | Aquilegia coerulea |
| NO: 606 | sequence of | Transporter | | | |
| SEQ ID | Amino acid | Uptake | T189_PsoPUP3_23 | From | Papaver |
| NO: 607 | sequence of | Transporter | | | somniferum |
| SEQ ID | DNA coding | Uptake | T189_PsoPUP3_23 | From | Papaver |
| NO: 608 | sequence of | Transporter | | | somniferum |
| SEQ ID | Amino acid | Uptake | T191_MdoPUP3_39 | From | Malus domestica |
| NO: 609 | sequence of | Transporter | | | |
| SEQ ID | DNA coding | Uptake | T191_MdoPUP3_39 | From | Malus domestica |
| NO: 610 | sequence of | Transporter | | | |
| SEQ ID | Amino acid | Uptake | T192_CmiPUP3_47 | From | Cinnamomum |
| NO: 611 | sequence of | Transporter | | | micranthum |
| | | | | | f. kanehirae |
| SEQ ID | DNA coding | Uptake | T192_CmiPUP3_47 | From | Cinnamomum |
| NO: 612 | sequence of | Transporter | | | micranthum |
| | | | | | f. kanehirae |
| SEQ ID | Amino acid | Uptake | T193_AanPUP3_55 | From | Artemisia annua |
| NO: 613 | sequence of | Transporter | | | |
| SEQ ID | DNA coding | Uptake | T193_AanPUP3_55 | From | Artemisia annua |
| NO: 614 | sequence of | Transporter | | | |
| SEQ ID | Amino acid | Uptake | T194_CchPUP3_63 | From | Capsicum chinense |
| NO: 615 | sequence of | Transporter | | | |
| SEQ ID | DNA coding | Uptake | T194_CchPUP3_63 | From | Capsicum chinense |
| NO: 616 | sequence of | Transporter | | | |
| SEQ ID | Amino acid | Uptake | T195_JcuPUP3_71 | From | Jatropha curcas |
| NO: 617 | sequence of | Transporter | | | |
| SEQ ID | DNA coding | Uptake | T195_JcuPUP3_71 | From | Jatropha curcas |
| NO: 618 | sequence of | Transporter | | | |
| SEQ ID | Amino acid | Uptake | T196_PtrPUP3_80 | From | Papaver trinifolium |
| NO: 619 | sequence of | Transporter | | | |
| SEQ ID | DNA coding | Uptake | T196_PtrPUP3_80 | From | Papaver trinifolium |
| NO: 620 | sequence of | Transporter | | | |
| SEQ ID | Amino acid | Uptake | T197_AcoT97_GA | From | Aquilegia coerulea |
| NO: 621 | sequence of | Transporter | | | |
| SEQ ID | DNA coding | Uptake | T197_AcoT97_GA | From | Aquilegia coerulea |
| NO: 622 | sequence of | Transporter | | | |
| SEQ ID | Amino acid | Uptake | T198_AcoT97_GA | From | Aquilegia coerulea |
| NO: 623 | sequence of | Transporter | | | |
| SEQ ID | DNA coding | Uptake | T198_AcoT97_GA | From | Aquilegia coerulea |
| NO: 624 | sequence of | Transporter | | | |
| SEQ ID | Amino acid | Uptake | T199_NnuT97_GA | From | Nelumbo nucifera |
| NO: 625 | sequence of | Transporter | | | |
| SEQ ID | DNA coding | Uptake | T199_NnuT97_GA | From | Nelumbo nucifera |
| NO: 626 | sequence of | Transporter | | | |
| SEQ ID | Amino acid | Uptake | T200_T97_GA | From | Prunus yedoensis |
| NO: 627 | sequence of | Transporter | | | var. nudiflora |
| SEQ ID | DNA coding | Uptake | T200_T97_GA | From | Prunus yedoensis |
| NO: 628 | sequence of | Transporter | | | var. nudiflora |
| SEQ ID | Amino acid | Uptake | T201_HarPUP3_GA | From | Helicoverpa |
| NO: 629 | sequence of | Transporter | | | armigera |
| SEQ ID | DNA coding | Uptake | T201_HarPUP3_GA | From | Helicoverpa |
| NO: 630 | sequence of | Transporter | | | armigera |
| SEQ ID | Amino acid | Uptake | T202_PgoPUP3_GA | From | Pectinophora |
| NO: 631 | sequence of | Transporter | | | gossypiella |
| SEQ ID | DNA coding | Uptake | T202_PgoPUP3_GA | From | Pectinophora |
| NO: 632 | sequence of | Transporter | | | gossypiella |
| SEQ ID | Amino acid | Uptake | T203_HarPUP3_GA | From | Helicoverpa |
| NO: 633 | sequence of | Transporter | | | armigera |
| SEQ ID | DNA coding | Uptake | T203_HarPUP3_GA | From | Helicoverpa |
| NO: 634 | sequence of | Transporter | | | armigera |
| SEQ ID | Amino acid | Uptake | T204_RcoPUP3_GA | From | Ricinus communis |
| NO: 635 | sequence of | Transporter | | | |
| SEQ ID | DNA coding | Uptake | T204_RcoPUP3_GA | From | Ricinus communis |
| NO: 636 | sequence of | Transporter | | | |
| SEQ ID | Amino acid | Uptake | T205_HviPUP3_GA | From | Heliothis virescens |
| NO: 637 | sequence of | Transporter | | | |
| SEQ ID | DNA coding | Uptake | T205_HviPUP3_GA | From | Heliothis virescens |
| NO: 638 | sequence of | Transporter | | | |
| SEQ ID | Amino acid | Uptake | T206_VviPUP3_3_GA | From | Vitis vinifera |
| NO: 639 | sequence of | Transporter | | | |
| SEQ ID | DNA coding | Uptake | T206_VviPUP3_3_GA | From | Vitis vinifera |
| NO: 640 | sequence of | Transporter | | | |
| SEQ ID | Amino acid | Uptake | T207_MprPUP3_GA | From | Mucuna pruriens |
| NO: 641 | sequence of | Transporter | | | |
| SEQ ID | DNA coding | Uptake | T207_MprPUP3_GA | From | Mucuna pruriens |
| NO: 642 | sequence of | Transporter | | | |
| SEQ ID | Amino acid | Uptake | T208_McoPUP3_GA | From | Macleaya cordata |
| NO: 643 | sequence of | Transporter | | | |
| SEQ ID | DNA coding | Uptake | T208_McoPUP3_GA | From | Macleaya cordata |
| NO: 644 | sequence of | Transporter | | | |
| SEQ ID | Amino acid | Uptake | T209_RcoPUP3_GA | From | Ricinus communis |
| NO: 645 | sequence of | Transporter | | | |
| SEQ ID | DNA coding | Uptake | T209_RcoPUP3_GA | From | Ricinus communis |
| NO: 646 | sequence of | Transporter | | | |
| SEQ ID | Amino acid | Uptake | T210_NnuPUP3_GA | From | Nelumbo nucifera |
| NO: 647 | sequence of | Transporter | | | |
| SEQ ID | DNA coding | Uptake | T210_NnuPUP3_GA | From | Nelumbo nucifera |
| NO: 648 | sequence of | Transporter | | | |
| SEQ ID | Amino acid | Uptake | T211_HarPUP3_GA | From | Helicoverpa |
| NO: 649 | sequence of | Transporter | | | armigera |
| SEQ ID | DNA coding | Uptake | T211_HarPUP3_GA | From | Helicoverpa |
| NO: 650 | sequence of | Transporter | | | armigera |
| SEQ ID | Amino acid | Uptake | T212_HarPUP3_GA | From | Helicoverpa |
| NO: 651 | sequence of | Transporter | | | armigera |
| SEQ ID | DNA coding | Uptake | T212_HarPUP3_GA | From | Helicoverpa |
| NO: 652 | sequence of | Transporter | | | armigera |
| SEQ ID | Amino acid | Uptake | T213_HarPUP3_GA | From | Helicoverpa |
| NO: 653 | sequence of | Transporter | | | armigera |
| SEQ ID | DNA coding | Uptake | T213_HarPUP3_GA | From | Helicoverpa |
| NO: 654 | sequence of | Transporter | | | armigera |
| SEQ ID | Amino acid | Uptake | T214_HarPUP3_GA | From | Helicoverpa |
| NO: 655 | sequence of | Transporter | | | armigera |
| SEQ ID | DNA coding | Uptake | T214 HarPUP3_GA | From | Helicoverpa |
| NO: 656 | sequence of | Transporter | | | armigera |
| SEQ ID | Amino acid | Uptake | T215_HarPUP3_GA | From | Helicoverpa |
| NO: 657 | sequence of | Transporter | | | armigera |
| SEQ ID | DNA coding | Uptake | T215_HarPUP3_GA | From | Helicoverpa |
| NO: 658 | sequence of | Transporter | | | armigera |
| SEQ ID | Amino acid | Uptake | T216_HarPUP3_GA | From | Helicoverpa |
| NO: 659 | sequence of | Transporter | | | armigera |
| SEQ ID | DNA coding | Uptake | T216_HarPUP3_GA | From | Helicoverpa |
| NO: 660 | sequence of | Transporter | | | armigera |
| SEQ ID | Amino acid | Uptake | T217_AcoPUP3_GA | From | Aquilegia coerulea |
| NO: 661 | sequence of | Transporter | | | |
| SEQ ID | DNA coding | Uptake | T217_AcoPUP3_GA | From | Aquilegia coerulea |
| NO: 662 | sequence of | Transporter | | | |
| SEQ ID | Amino acid | Uptake | T65_ljaNPF_GA | From | Lonicera japonica |
| NO: 733 | sequence of | Transporter | | | |
| SEQ ID | DNA coding | Uptake | T65_ljaNPF_GA | From | Lonicera japonica |
| NO: 734 | sequence of | Transporter | | | |
| SEQ ID | Amino acid | Uptake | T94_EcrPOT_GA | From | Emmonsia |
| NO: 735 | sequence of | Transporter | | | crescens |
| SEQ ID | DNA coding | Uptake | T94_EcrPOT_GA | From | Emmonsia |
| NO: 736 | sequence of | Transporter | | | crescens |
| SEQ ID | Amino acid | ADH5 | ADH5 | From | Saccharomyces |
| NO: 663 | sequence of | Dehydrogenase | Dehydrogenase | | cerevisiae |
| SEQ ID | DNA coding | ADH5 | ADH5 | From | Saccharomyces |
| NO: 664 | sequence of | Dehydrogenase | Dehydrogenase | | cerevisiae |
| SEQ ID | Amino acid | ADH6 | ADH6 | From | Saccharomyces |
| NO: 665 | sequence of | Dehydrogenase | Dehydrogenase | | cerevisiae |
| SEQ ID | DNA coding | ADH6 | ADH6 | From | Saccharomyces |
| NO: 666 | sequence of | Dehydrogenase | Dehydrogenase | | cerevisiae |
| SEQ ID | Amino acid | ADH7 | ADH7 | From | Saccharomyces |
| NO: 667 | sequence of | Dehydrogenase | Dehydrogenase | | cerevisiae |
| SEQ ID | DNA coding | ADH7 | ADH7 | From | Saccharomyces |
| NO: 668 | sequence of | Dehydrogenase | Dehydrogenase | | cerevisiae |
| SEQ ID | Amino acid | YPR127W | YPR127W | From | Saccharomyces |
| NO: 669 | sequence of | Dehydrogenase | Dehydrogenase | | cerevisiae |
| SEQ ID | DNA coding | YPR127W | YPR127W | From | Saccharomyces |
| NO: 670 | sequence of | Dehydrogenase | Dehydrogenase | | cerevisiae |
| SEQ ID | Amino acid | AAD3 | AAD3 | From | Saccharomyces |
| NO: 671 | sequence of | Dehydrogenase | Dehydrogenase | | cerevisiae |
| SEQ ID | DNA coding | AAD3 | AAD3 | From | Saccharomyces |
| NO: 672 | sequence of | Dehydrogenase | Dehydrogenase | | cerevisiae |
| SEQ ID | Amino acid | AAD4 | AAD4 | From | Saccharomyces |
| NO: 673 | sequence of | Dehydrogenase | Dehydrogenase | | cerevisiae |
| SEQ ID | DNA coding | AAD4 | AAD4 | From | Saccharomyces |
| NO: 674 | sequence of | Dehydrogenase | Dehydrogenase | | cerevisiae |
| SEQ ID | Amino acid | ADH3 | ADH3 | From | Saccharomyces |
| NO: 675 | sequence of | Dehydrogenase | Dehydrogenase | | cerevisiae |
| SEQ ID | DNA coding | ADH3 | ADH3 | From | Saccharomyces |
| NO: 676 | sequence of | Dehydrogenase | Dehydrogenase | | cerevisiae |
| SEQ ID | Amino acid | ADH4 | ADH4 | From | Saccharomyces |
| NO: 677 | sequence of | Dehydrogenase | Dehydrogenase | | cerevisiae |
| SEQ ID | DNA coding | ADH4 | ADH4 | From | Saccharomyces |
| NO: 678 | sequence of | Dehydrogenase | Dehydrogenase | | cerevisiae |
| SEQ ID | Amino acid | ALD6 | ALD6 | From | Saccharomyces |
| NO: 679 | sequence of | Dehydrogenase | Dehydrogenase | | cerevisiae |
| SEQ ID | DNA coding | ALD6 | ALD6 | From | Saccharomyces |
| NO: 680 | sequence of | Dehydrogenase | Dehydrogenase | | cerevisiae |
| SEQ ID | Amino acid | BDH1 | BDH1 | From | Saccharomyces |
| NO: 681 | sequence of | Dehydrogenase | Dehydrogenase | | cerevisiae |
| SEQ ID | DNA coding | BDH1 | BDH1 | From | Saccharomyces |
| NO: 682 | sequence of | Dehydrogenase | Dehydrogenase | | cerevisiae |
| SEQ ID | Amino acid | BDH2 | BDH2 | From | Saccharomyces |
| NO: 683 | sequence of | Dehydrogenase | Dehydrogenase | | cerevisiae |
| SEQ ID | DNA coding | BDH2 | BDH2 | From | Saccharomyces |
| NO: 684 | sequence of | Dehydrogenase | Dehydrogenase | | cerevisiae |
| SEQ ID | Amino acid | FOX2 | FOX2 | From | Saccharomyces |
| NO: 685 | sequence of | Dehydrogenase | Dehydrogenase | | cerevisiae |
| SEQ ID | DNA coding | FOX2 | FOX2 | From | Saccharomyces |
| NO: 686 | sequence of | Dehydrogenase | Dehydrogenase | | cerevisiae |
| SEQ ID | Amino acid | GCY1 | GCY1 | From | Saccharomyces |
| NO: 687 | sequence of | Dehydrogenase | Dehydrogenase | | cerevisiae |
| SEQ ID | DNA coding | GCY1 | GCY1 | From | Saccharomyces |
| NO: 688 | sequence of | Dehydrogenase | Dehydrogenase | | cerevisiae |
| SEQ ID | Amino acid | GPD1 | GPD1 | From | Saccharomyces |
| NO: 689 | sequence of | Dehydrogenase | Dehydrogenase | | cerevisiae |
| SEQ ID | DNA coding | GPD1 | GPD1 | From | Saccharomyces |
| NO: 690 | sequence of | Dehydrogenase | Dehydrogenase | | cerevisiae |
| SEQ ID | Amino acid | HIS4 | HIS4 Dehydrogenase | From | Saccharomyces |
| NO: 691 | sequence of | Dehydrogenase | | | cerevisiae |
| SEQ ID | DNA coding | HIS4 | HIS4 Dehydrogenase | From | Saccharomyces |
| NO: 692 | sequence of | Dehydrogenase | | | cerevisiae |
| SEQ ID | Amino acid | IPD1 | IPD1 Dehydrogenase | From | Saccharomyces |
| NO: 693 | sequence of | Dehydrogenase | | | cerevisiae |
| SEQ ID | DNA coding | IPD1 | IPD1 Dehydrogenase | From | Saccharomyces |
| NO: 694 | sequence of | Dehydrogenase | | | cerevisiae |
| SEQ ID | Amino acid | LYS12 | LYS12 | From | Saccharomyces |
| NO: 695 | sequence of | Dehydrogenase | Dehydrogenase | | cerevisiae |
| SEQ ID | DNA coding | LYS12 | LYS12 | From | Saccharomyces |
| NO: 696 | sequence of | Dehydrogenase | Dehydrogenase | | cerevisiae |
| SEQ ID | Amino acid | SER33 | SER33 | From | Saccharomyces |
| NO: 697 | sequence of | Dehydrogenase | Dehydrogenase | | cerevisiae |
| SEQ ID | DNA coding | SER33 | SER33 | From | Saccharomyces |
| NO: 698 | sequence of | Dehydrogenase | Dehydrogenase | | cerevisiae |
| SEQ ID | Amino acid | ZWF1 | ZWF1 | From | Saccharomyces |
| NO: 699 | sequence of | Dehydrogenase | Dehydrogenase | | cerevisiae |
| SEQ ID | DNA coding | ZWF1 | ZWF1 | From | Saccharomyces |
| NO: 700 | sequence of | Dehydrogenase | Dehydrogenase | | cerevisiae |
| SEQ ID | Amino acid | YPL088W | YPL088W | From | Saccharomyces |
| NO: 701 | sequence of | Dehydrogenase | Dehydrogenase | | cerevisiae |
| SEQ ID | DNA coding | YPL088W | YPL088W | From | Saccharomyces |
| NO: 702 | sequence of | Dehydrogenase | Dehydrogenase | | cerevisiae |
| SEQ ID | Amino acid | ARA1 | ARA1 | From | Saccharomyces |
| NO: 703 | sequence of | Dehydrogenase | Dehydrogenase | | cerevisiae |
| SEQ ID | DNA coding | ARA1 | ARA1 | From | Saccharomyces |
| NO: 704 | sequence of | Dehydrogenase | Dehydrogenase | | cerevisiae |
| SEQ ID | Amino acid | HFD1 | HFD1 | From | Saccharomyces |
| NO: 705 | sequence of | Dehydrogenase | Dehydrogenase | | cerevisiae |
| SEQ ID | DNA coding | HFD1 | HFD1 | From | Saccharomyces |
| NO: 706 | sequence of | Dehydrogenase | Dehydrogenase | | cerevisiae |
| SEQ ID | Amino acid | YPR1 | YPR1 Reductase | From | Saccharomyces |
| NO: 707 | sequence of | Reductase | | | cerevisiae |
| SEQ ID | DNA coding | YPR1 | YPR1 Reductase | From | Saccharomyces |
| NO: 708 | sequence of | Reductase | | | cerevisiae |
| SEQ ID | Amino acid | ALD4 | ALD4 Reductase | From | Saccharomyces |
| NO: 709 | sequence of | Reductase | | | cerevisiae |
| SEQ ID | DNA coding | ALD4 | ALD4 Reductase | From | Saccharomyces |
| NO: 710 | sequence of | Reductase | | | cerevisiae |
| SEQ ID | Amino acid | GOR1 | GOR1 Reductase | From | Saccharomyces |
| NO: 711 | sequence of | Reductase | | | cerevisiae |
| SEQ ID | DNA coding | GOR1 | GOR1 Reductase | From | Saccharomyces |
| NO: 712 | sequence of | Reductase | | | cerevisiae |
| SEQ ID | Amino acid | GRE2 | GRE2 Reductase | From | Saccharomyces |
| NO: 713 | sequence of | Reductase | | | cerevisiae |
| SEQ ID | DNA coding | GRE2 | GRE2 Reductase | From | Saccharomyces |
| NO: 714 | sequence of | Reductase | | | cerevisiae |
| SEQ ID | Amino acid | GRE3 | GRE3 Reductase | From | Saccharomyces |
| NO: 715 | sequence of | Reductase | | | cerevisiae |
| SEQ ID | DNA coding | GRE3 | GRE3 Reductase | From | Saccharomyces |
| NO: 716 | sequence of | Reductase | | | cerevisiae |
| SEQ ID | Amino acid | YDR541C | YDR541C Reductase | From | Saccharomyces |
| NO: 717 | sequence of | Reductase | | | cerevisiae |
| SEQ ID | DNA coding | YDR541C | YDR541C Reductase | From | Saccharomyces |
| NO: 718 | sequence of | Reductase | | | cerevisiae |
| SEQ ID | Amino acid | YLR460C | YLR460C Reductase | From | Saccharomyces |
| NO: 719 | sequence of | Reductase | | | cerevisiae |
| SEQ ID | DNA coding | YLR460C | YLR460C Reductase | From | Saccharomyces |
| NO: 720 | sequence of | Reductase | | | cerevisiae |
| SEQ ID | Amino acid | ARI1 | ARI1 Reductase | From | Saccharomyces |
| NO: 721 | sequence of | Reductase | | | cerevisiae |
| SEQ ID | DNA coding | ARI1 | ARI1 Reductase | From | Saccharomyces |
| NO: 722 | sequence of | Reductase | | | cerevisiae |
| SEQ ID | Amino acid | YGL039W | YGL039W Reductase | From | Saccharomyces |
| NO: 723 | sequence of | Reductase | | | cerevisiae |
| SEQ ID | DNA coding | YGL039W | YGL039W Reductase | From | Saccharomyces |
| NO: 724 | sequence of | Reductase | | | cerevisiae |
| SEQ ID | Amino acid | YCR102C | YCR102C Reductase | From | Saccharomyces |
| NO: 725 | sequence of | Reductase | | | cerevisiae |
| SEQ ID | DNA coding | YCR102C | YCR102C Reductase | From | Saccharomyces |
| NO: 726 | sequence of | Reductase | | | cerevisiae |
| SEQ ID | Amino acid | HMG1 | HMG1 Reductase | From | Saccharomyces |
| NO: 727 | sequence of | Reductase | | | cerevisiae |
| SEQ ID | DNA coding | HMG1 | HMG1 Reductase | From | Saccharomyces |
| NO: 728 | sequence of | Reductase | | | cerevisiae |
| SEQ ID | Amino acid | PHA2 | PHA2 Dehydratase | From | Saccharomyces |
| NO: 729 | sequence of | Dehydratase | | | cerevisiae |
| SEQ ID | DNA coding | PHA2 | PHA2 Dehydratase | From | Saccharomyces |
| NO: 730 | sequence of | Dehydratase | | | cerevisiae |
| SEQ ID | Amino acid | TRP3 | TRP3 Synthase | From | Saccharomyces |
| NO: 731 | sequence of | Synthase | | | cerevisiae |
| SEQ ID | DNA coding | TRP3 | TRP3 Synthase | From | Saccharomyces |
| NO: 732 | sequence of | Synthase | | | cerevisiae |
| SEQ ID | DNA coding | HEME | HEM2 | From | Saccharomyces |
| NO: 737 | sequence of | cofactor | | | cerevisiae |
| SEQ ID | Amino acid | HEME | HEM2 | From | Saccharomyces |
| NO: 738 | sequence of | cofactor | | | cerevisiae |
| SEQ ID | DNA coding | HEME | HEM3 | From | Saccharomyces |
| NO: 739 | sequence of | cofactor | | | cerevisiae |
| SEQ ID | Amino acid | HEME | HEM3 | From | Saccharomyces |
| NO: 740 | sequence of | cofactor | | | cerevisiae |
| SEQ ID | DNA coding | HEME | HEM12 | From | Saccharomyces |
| NO: 741 | sequence of | cofactor | | | cerevisiae |
| SEQ ID | Amino acid | HEME | HEM12 | From | Saccharomyces |
| NO: 742 | sequence of | cofactor | | | cerevisiae |
| SEQ ID | DNA coding | HEME | HMX1 | From | Saccharomyces |
| NO: 743 | sequence of | cofactor | | | cerevisiae |
| SEQ ID | Amino acid | HEME | HMX1 | From | Saccharomyces |
| NO: 744 | sequence of | cofactor | | | cerevisiae |
| SEQ ID | DNA coding | P450 | KAR2 | From | Saccharomyces |
| NO: 745 | sequence of | chaperone | | | cerevisiae |
| SEQ ID | Amino acid | P450 | KAR2 | From | Saccharomyces |
| NO: 746 | sequence of | chaperone | | | cerevisiae |
| SEQ ID | DNA coding | P450 | HSP82 | From | Saccharomyces |
| NO: 747 | sequence of | chaperone | | | cerevisiae |
| SEQ ID | Amino acid | P450 | HSP82 | From | Saccharomyces |
| NO: 748 | sequence of | chaperone | | | cerevisiae |
| SEQ ID | DNA coding | P450 | CNE1 | From | Saccharomyces |
| NO: 749 | sequence of | chaperone | | | cerevisiae |
| SEQ ID | Amino acid | P450 | CNE1 | From | Saccharomyces |
| NO: 750 | sequence of | chaperone | | | cerevisiae |
| SEQ ID | DNA coding | P450 | SSA1 | From | Saccharomyces |
| NO: 751 | sequence of | chaperone | | | cerevisiae |
| SEQ ID | Amino acid | P450 | SSA1 | From | Saccharomyces |
| NO: 752 | sequence of | chaperone | | | cerevisiae |
| SEQ ID | DNA coding | P450 | CPR6 | From | Saccharomyces |
| NO: 753 | sequence of | chaperone | | | cerevisiae |
| SEQ ID | Amino acid | P450 | CPR6 | From | Saccharomyces |
| NO: 754 | sequence of | chaperone | | | cerevisiae |
| SEQ ID | DNA coding | P450 | FES1 | From | Saccharomyces |
| NO: 755 | sequence of | chaperone | | | cerevisiae |
| SEQ ID | Amino acid | P450 | FES1 | From | Saccharomyces |
| NO: 756 | sequence of | chaperone | | | cerevisiae |
| SEQ ID | DNA coding | P450 | HSP104 | From | Saccharomyces |
| NO: 757 | sequence of | chaperone | | | cerevisiae |
| SEQ ID | Amino acid | P450 | HSP104 | From | Saccharomyces |
| NO: 758 | sequence of | chaperone | | | cerevisiae |
| SEQ ID | DNA coding | P450 | STI1 | From | Saccharomyces |
| NO: 759 | sequence of | chaperone | | | cerevisiae |
| SEQ ID | Amino acid | P450 | STI1 | From | Saccharomyces |
| NO: 760 | sequence of | chaperone | | | cerevisiae |
| SEQ ID | DNA coding | P450 | DAP1 | From | Saccharomyces |
| NO: 761 | sequence of | regulator | | | cerevisiae |
| SEQ ID | Amino acid | P450 | DAP1 | From | Saccharomyces |
| NO: 762 | sequence of | regulator | | | cerevisiae |
| SEQ ID | DNA coding | P450 | HAC1 | From | Saccharomyces |
| NO: 763 | sequence of | regulator | | | cerevisiae |
| SEQ ID | Amino acid | P450 | HAC1 | From | Saccharomyces |
| NO: 764 | sequence of | regulator | | | cerevisiae |
| SEQ ID | DNA coding | NADPH | ZWF1 | From | Saccharomyces |
| NO: 765 | sequence of | cofactor | | | cerevisiae |
| SEQ ID | Amino acid | NADPH | ZWF1 | From | Saccharomyces |
| NO: 766 | sequence of | cofactor | | | cerevisiae |
| SEQ ID | DNA coding | NADPH | GND1 | From | Saccharomyces |
| NO: 767 | sequence of | cofactor | | | cerevisiae |
| SEQ ID | Amino acid | NADPH | GND1 | From | Saccharomyces |
| NO: 768 | sequence of | cofactor | | | cerevisiae |
| SEQ ID | DNA coding | formadehyde | SFA1 | From | Saccharomyces |
| NO: 769 | sequence of | toxicity | | | cerevisiae |
| | | regulator | | | |
| SEQ ID | Amino acid | formadehyde | SFA1 | From | Saccharomyces |
| NO: 770 | sequence of | toxicity | | | cerevisiae |
| | | regulator | | | |
| SEQ ID | DNA coding | P450 | — | From | Artificial |
| NO: 771 | sequence of | (demethylase) | | | |
| SEQ ID | Amino acid | P450 | — | From | Artificial |
| NO: 772 | sequence of | (demethylase) | | | |
| SEQ ID | DNA coding | Uptake | T149_AcPUP3_59_co2 | From | Artificial |
| NO: 773 | sequence of | transporter | | | |
| | | codon | | | |
| | | optimised | | | |
| SEQ ID | Amino acid | Uptake | T149_AcPUP3_59_co2 | From | Aquilegia coerulea |
| NO: 774 | sequence of | transporter | | | |
| SEQ ID | DNA coding | Uptake | T149_AcPUP3_59_co3 | From | Artificial |
| NO: 775 | sequence of | transporter | | | |
| | | codon | | | |
| | | optimised | | | |
| SEQ ID | Amino acid | Uptake | T149_AcPUP3_59_co3 | From | Aquilegia coerulea |
| NO: 776 | sequence of | transporter | | | |
| SEQ ID | DNA coding | Uptake | T149_AcPUP3_59_co4 | From | Artificial |
| NO: 777 | sequence of | transporter | | | |
| | | codon | | | |
| | | optimised | | | |
| SEQ ID | Amino acid | Uptake | T149_AcPUP3_59_co4 | From | Aquilegia coerulea |
| NO: 778 | sequence of | transporter | | | |
| | | transporter | | | |
| SEQ ID | DNA coding | Uptake | T180_McPUP3_46_co2 | From | Artificial |
| NO: 779 | sequence of | transporter | | | |
| | | transporter | | | |
| | | codon | | | |
| | | optimised | | | |
| SEQ ID | Amino acid | Uptake | T180_McPUP3_46_co2 | From | Momordica |
| NO: 780 | sequence of | transporter | | | charantia |
| SEQ ID | DNA coding | Uptake | T180_McPUP3_46_co3 | From | Artificial |
| NO: 781 | sequence of | transporter | | | |
| | | codon | | | |
| | | optimised | | | |
| SEQ ID | Amino acid | Uptake | T180_McPUP3_46_co3 | From | Momordica |
| NO: 782 | sequence of | transporter | | | charantia |
| SEQ ID | DNA coding | Uptake | T180_McPUP3_46_co4 | From | Artificial |
| NO: 783 | sequence of | transporter | | | |
| | | codon | | | |
| | | optimised | | | |
| SEQ ID | Amino acid | Uptake | T180_McPUP3_46_co4 | From | Momordica |
| NO: 784 | sequence of | transporter | | | charantia |
| SEQ ID | DNA coding | Uptake | T180_McPUP3_46_co6 | From | Artificial |
| NO: 785 | sequence of | transporter | | | |
| | | codon | | | |
| | | optimised | | | |
| SEQ ID | Amino acid | Uptake | T180_McPUP3_46_co6 | From | Momordica |
| NO: 786 | sequence of | transporter | | | charantia |
| SEQ ID | DNA coding | Uptake | T193_AanPUP3_55_co2 | From | Artificial |
| NO: 787 | sequence of | transporter | | | |
| | | codon | | | |
| | | optimised | | | |
| SEQ ID | Amino acid | Uptake | T193_AanPUP3_55_co2 | From | Artemisia annua |
| NO: 788 | sequence of | transporter | | | |
| SEQ ID | DNA coding | Uptake | T193_AanPUP3_55_co3 | From | Artificial |
| NO: 789 | sequence of | transporter | | | |
| | | codon | | | |
| | | optimised | | | |
| SEQ ID | Amino acid | Uptake | T193_AanPUP3_55_co3 | From | Artemisia annua |
| NO: 790 | sequence of | transporter | | | |
| SEQ ID | DNA coding | Uptake | T193_AanPUP3_55_co5 | From | Artificial |
| NO: 791 | sequence of | transporter | | | |
| | | codon | | | |
| | | optimised | | | |
| SEQ ID | Amino acid | Uptake | T193_AanPUP3_55_co5 | From | Artemisia annua |
| NO: 792 | sequence of | transporter | | | |
| SEQ ID | DNA coding | Uptake | T193_AanPUP3_55_co6 | From | Artificial |
| NO: 793 | sequence of | transporter | | | |
| | | codon | | | |
| | | optimised | | | |
| SEQ ID | Amino acid | Uptake | T193_AanPUP3_55_co6 | From | Artemisia annua |
| NO: 794 | sequence of | transporter | | | |
| SEQ ID | Amino acid | Uptake | T218_HviENT3_GA | From | Heliothis virescens |
| NO: 795 | sequence of | transporter | | | |
| SEQ ID | DNA coding | Uptake | T218_HviENT3_GA | From | Heliothis virescens |
| NO: 796 | sequence of | transporter | | | |
| SEQ ID | Amino acid | Uptake | T220_CsuENT3_GA | From | Chilo suppressalis |
| NO: 797 | sequence of | transporter | | | |
| SEQ ID | DNA coding | Uptake | T220_CsuENT3_GA | From | Chilo suppressalis |
| NO: 798 | sequence of | transporter | | | |
| SEQ ID | Amino acid | Uptake | T221_BmoENT3_GA | From | Bombyx mori |
| NO: 799 | sequence of | transporter | | | |
| SEQ ID | DNA coding | Uptake | T221_BmoENT3_GA | From | Bombyx mori |
| NO: 800 | sequence of | transporter | | | |
| SEQ ID | Amino acid | Uptake | T227_AcuENT3_GA | From | Anopheles |
| NO: 801 | sequence of | transporter | | | culicifacies |
| SEQ ID | DNA coding | Uptake | T227_AcuENT3_GA | From | Anopheles |
| NO: 802 | sequence of | transporter | | | culicifacies |
| SEQ ID | Amino acid | Uptake | T234_CsuENT3_GA | From | Chilo suppressalis |
| NO: 803 | sequence of | transporter | | | |
| SEQ ID | DNA coding | Uptake | T234_CsuENT3_GA | From | Chilo suppressalis |
| NO: 804 | sequence of | transporter | | | |
| SEQ ID | Amino acid | Uptake | T237_PxuENT3_GA | From | Papilio xuthus |
| NO: 805 | sequence of | transporter | | | |
| SEQ ID | DNA coding | Uptake | T237_PxuENT3_GA | From | Papilio xuthus |
| NO: 806 | sequence of | transporter | | | |
| SEQ ID | Amino acid | Uptake | T238_HviENT3_GA | From | Heliothis virescens |
| NO: 807 | sequence of | transporter | | | |
| SEQ ID | DNA coding | Uptake | T238_HviENT3_GA | From | Heliothis virescens |
| NO: 808 | sequence of | transporter | | | |
| SEQ ID | Amino acid | Uptake | T239_CmePUP3_GA | From | Cucumis melo var. |
| NO: 809 | sequence of | transporter | | | makuwa |
| SEQ ID | DNA coding | Uptake | T239_CmePUP3_GA | From | Cucumis melo var. |
| NO: 810 | sequence of | transporter | | | makuwa |
| SEQ ID | Amino acid | Uptake | T240_PpePUP3_GA | From | Prunus persica |
| NO: 811 | sequence of | transporter | | | |
| SEQ ID | DNA coding | Uptake | T240_PpePUP3_GA | From | Prunus persica |
| NO: 812 | sequence of | transporter | | | |
| SEQ ID | Amino acid | Uptake | T242_AchPUP3_GA | From | Actinidia chinensis |
| NO: 813 | sequence of | transporter | | | var. chinensis |
| SEQ ID | DNA coding | Uptake | T242_AchPUP3_GA | From | Actinidia chinensis |
| NO: 814 | sequence of | transporter | | | var. chinensis |
| SEQ ID | Amino acid | Uptake | T243_EguPUP3_GA | From | Erythranthe guttata |
| NO: 815 | sequence of | transporter | | | |
| SEQ ID | DNA coding | Uptake | T243_EguPUP3_GA | From | Erythranthe guttata |
| NO: 816 | sequence of | transporter | | | |
| SEQ ID | Amino acid | Uptake | T244_CcaPUP3_GA | From | Corchorus |
| NO: 817 | sequence of | transporter | | | capsularis |
| SEQ ID | DNA coding | Uptake | T244_CcaPUP3_GA | From | Corchorus |
| NO: 818 | sequence of | transporter | | | capsularis |
| SEQ ID | Amino acid | Uptake | T245_CcaPUP3_GA | From | Handroanthus |
| NO: 819 | sequence of | transporter | | | impetiginosus |
| SEQ ID | DNA coding | Uptake | T245_CcaPUP3_GA | From | Handroanthus |
| NO: 820 | sequence of | transporter | | | impetiginosus |
| SEQ ID | Amino acid | Uptake | T248_McoPUP3_GA | From | Macleaya cordata |
| NO: 821 | sequence of | transporter | | | |
| SEQ ID | DNA coding | Uptake | T248_McoPUP3_GA | From | Macleaya cordata |
| NO: 822 | sequence of | transporter | | | |
| SEQ ID | Amino acid | Uptake | T253_AanPUP3_GA | From | Artemisia annua |
| NO: 823 | sequence of | transporter | | | |
| SEQ ID | DNA coding | Uptake | T253_AanPUP3_GA | From | Artemisia annua |
| NO: 824 | sequence of | transporter | | | |
| SEQ ID | Amino acid | Uptake | T254_CcaPUP3_GA | From | Cynara |
| NO: 825 | sequence of | transporter | | | cardunculus var. |
| | | | | | scolymus |
| SEQ ID | DNA coding | Uptake | T254_CcaPUP3_GA | From | Cynara |
| NO: 826 | sequence of | transporter | | | cardunculus var. |
| | | | | | scolymus |
| SEQ ID | Amino acid | P450 | A0A286QUG7 | From | Spodoptera exigua |
| NO: 827 | sequence of | (demethylase) | | | |
| SEQ ID | DNA coding | P450 | A0A286QUG7 | From | Spodoptera exigua |
| NO: 828 | sequence of | (demethylase) | | | |
| SEQ ID | Amino acid | P450 | D5L0M5 | From | Manduca sexta |
| NO: 829 | sequence of | (demethylase) | | | |
| SEQ ID | DNA coding | P450 | D5L0M5 | From | Manduca sexta |
| NO: 830 | sequence of | (demethylase) | | | |
| SEQ ID | Amino acid | P450 | XP026740610 | From | Trichoplusia ni |
| NO: 831 | sequence of | (demethylase) | | | |
| SEQ ID | DNA coding | P450 | XP026740610 | From | Trichoplusia ni |
| NO: 832 | sequence of | (demethylase) | | | |
| SEQ ID | Amino acid | P450 | W5W4U7 | From | Lymantria dispar |
| NO: 833 | sequence of | (demethylase) | | | |
| SEQ ID | DNA coding | P450 | W5W4U7 | From | Lymantria dispar |
| NO: 834 | sequence of | (demethylase) | | | |
| SEQ ID | Amino acid | P450 | ACF17813 | From | Ostrinia furnacalis |
| NO: 835 | sequence of | (demethylase) | | | |
| SEQ ID | DNA coding | P450 | ACF17813 | From | Ostrinia furnacalis |
| NO: 836 | sequence of | (demethylase) | | | |
| SEQ ID | Amino acid | P450 | A0A4C1YMA7 | From | Eumeta variegata |
| NO: 837 | sequence of | (demethylase) | | | |
| SEQ ID | DNA coding | P450 | A0A4C1YMA7 | From | Eumeta variegata |
| NO: 838 | sequence of | (demethylase) | | | |
| SEQ ID | Amino acid | P450 | MsCPR_XP_030039194 | From | Manduca sexta |
| NO: 839 | sequence of | (demethylase) | | | |
| SEQ ID | DNA coding | P450 | MsCPR_XP_030039194 | From | Manduca sexta |
| NO: 840 | sequence of | (demethylase) | | | |
| SEQ ID | Amino acid | P450 | HvCPR_A0A2A4IYH3 | From | Heliothis virescens |
| NO: 841 | sequence of | (demethylase) | | | |
| SEQ ID | DNA coding | P450 | HvCPR_A0A2A4IYH3 | From | Heliothis virescens |
| NO: 842 | sequence of | (demethylase) | | | |
| SEQ ID | Amino acid | P450 | HaCYP6AE15v2_t | From | Helicoverpa |
| NO: 843 | sequence of | (demethylase) | | | armigera |
| SEQ ID | DNA coding | P450 | HaCYP6AE15v2_t | From | Helicoverpa |
| NO: 844 | sequence of | (demethylase) | | | armigera |
| SEQ ID | Amino acid | P450 | NMCH- | From | Helicoverpa |
| NO: 845 | sequence of | (demethylase) | HaCYP6AE15v2_t | | armigera |
| | | | (Amino acid 1-25 -> | | |
| | | | NMCH N-terminal | | |
| | | | signal peptide) | | |
| SEQ ID | DNA coding | P450 | NMCH- | From | Helicoverpa |
| NO: 846 | sequence of | (demethylase) | HaCYP6AE15v2_t | | armigera |
| SEQ ID | Amino acid | P450 | EcCFS-SP- | From | Helicoverpa |
| NO: 847 | sequence of | (demethylase) | HaCYP6AE15v2_t | | armigera |
| | | | (Amino acid 1-22 -> | | |
| | | | EcCFS N-terminal | | |
| | | | signal peptide) | | |
| SEQ ID | DNA coding | P450 | EcCFS-SP- | From | Helicoverpa |
| NO: 848 | sequence of | (demethylase) | HaCYP6AE15v2_t | | armigera |
| SEQ ID | Amino acid | P450 | HaCYP6AE15v2_A316G_t | From | Helicoverpa |
| NO: 849 | sequence of | (demethylase) | | | armigera |
| SEQ ID | DNA coding | P450 | HaCYP6AE15v2_A316G_t | From | Helicoverpa |
| NO: 850 | sequence of | (demethylase) | | | armigera |
| SEQ ID | Amino acid | P450 | NMCH- | From | Helicoverpa |
| NO: 851 | sequence of | (demethylase) | HaCYP6AE15v2_A316G_t | | armigera |
| | | | (Amino acid 1- | | |
| | | | 25 ->NMCH N- | | |
| | | | terminal signal | | |
| | | | peptide) | | |
| SEQ ID | DNA coding | P450 | NMCH- | From | Helicoverpa |
| NO: 852 | sequence of | (demethylase) | HaCYP6AE15v2_A316G_t | | armigera |
| SEQ ID | Amino acid | P450 | EcCFS-SP- | From | Helicoverpa |
| NO: 853 | sequence of | (demethylase) | HaCYP6AE15v2_A316G_t | | armigera |
| | | | (Amino acid 1- | | |
| | | | 22 -> EcCFS N- | | |
| | | | terminal signal | | |
| | | | peptide) | | |
| SEQ ID | DNA coding | P450 | EcCFS-SP- | From | Helicoverpa |
| NO: 854 | sequence of | (demethylase) | HaCYP6AE15v2_A316G_t | | armigera |
| SEQ ID | Amino acid | P450 | HaCYP6AE15v2_D392E_t | From | Helicoverpa |
| NO: 855 | sequence of | (demethylase) | | | armigera |
| SEQ ID | DNA coding | P450 | HaCYP6AE15v2_D392E_t | From | Helicoverpa |
| NO: 856 | sequence of | (demethylase) | | | armigera |
| SEQ ID | Amino acid | P450 | NMCH- | From | Helicoverpa |
| NO: 857 | sequence of | (demethylase) | HaCYP6AE15v2_D392E_t | | armigera |
| | | | (Amino acid 1- | | |
| | | | 25 -> NMCH N- | | |
| | | | terminal signal | | |
| | | | peptide) | | |
| SEQ ID | DNA coding | P450 | NMCH- | From | Helicoverpa |
| NO: 858 | sequence of | (demethylase) | HaCYP6AE15v2_D392E_t | | armigera |
| SEQ ID | Amino acid | P450 | EcCFS-SP- | From | Helicoverpa |
| NO: 859 | sequence of | (demethylase) | HaCYP6AE15v2_D392E_t | | armigera |
| | | | (Amino acid 1- | | |
| | | | 22 -> EcCFS N- | | |
| | | | terminal signal | | |
| | | | peptide) | | |
| SEQ ID | DNA coding | P450 | EcCFS-SP- | From | Helicoverpa |
| NO: 860 | sequence of | (demethylase) | HaCYP6AE15v2_D392E_t | | armigera |
| SEQ ID | Amino acid | P450 | Hv_CYP_A0A2A4JAM9_t | From | Heliothis virescens |
| NO:861 | sequence of | (demethylase) | | | |
| SEQ ID | DNA coding | P450 | Hv_CYP_A0A2A4JAM9_t | From | Heliothis virescens |
| NO: 862 | sequence of | (demethylase) | | | |
| SEQ ID | Amino acid | P450 | NMCH- | From | Heliothis virescens |
| NO: 863 | sequence of | (demethylase) | Hv_CYP_A0A2A4JAM9_t | | |
| | | | (Amino acid 1- | | |
| | | | 25 -> NMCH N- | | |
| | | | terminal signal | | |
| | | | peptide) | | |
| SEQ ID | DNA coding | P450 | NMCH- | From | Heliothis virescens |
| NO: 864 | sequence of | (demethylase) | Hv_CYP_A0A2A4JAM9_t | | |
| SEQ ID | Amino acid | P450 | EcCFS-SP- | From | Heliothis virescens |
| NO: 865 | sequence of | (demethylase) | Hv_CYP_A0A2A4JAM9_t | | |
| | | | (Amino acid 1- | | |
| | | | 22 -> EcCFS N- | | |
| | | | terminal signal | | |
| | | | peptide) | | |
| SEQ ID | DNA coding | P450 | EcCFS-SP- | From | Heliothis virescens |
| NO: 866 | sequence of | (demethylase) | Hv_CYP_A0A2A4JAM9_t | | |
| SEQ ID | Amino acid | P450 | EcCFS (CYP719A5) | From | Eschscholzia |
| NO: 867 | sequence of | (demethylase) | (Amino acid 1-22 -> | | californica |
| | | | N-terminal signal | | |
| | | | peptide) | | |
| SEQ ID | DNA coding | P450 | EcCFS (CYP719A5) | From | Eschscholzia |
| NO: 868 | sequence of | (demethylase) | | | californica |
| SEQ ID | Amino acid | P450 | EcNMCH (CYP80B2) | From | Eschscholzia |
| NO: 869 | sequence of | (demethylase) | (Amino acid 1-25 -> | | californica |
| | | | N-terminal signal | | |
| | | | peptide) | | |
| SEQ ID | DNA coding | P450 | EcNMCH (CYP80B2) | From | Eschscholzia |
| NO: 870 | sequence of | (demethylase) | | | californica |
| SEQ ID | DNA coding | Efflux | YOR1 | From | Saccharomyces |
| NO: 871 | sequence of | transporter | | | cerevisiae |
| SEQ ID | Amino acid | Efflux | YOR1 | From | Saccharomyces |
| NO: 872 | sequence of | transporter | | | cerevisiae |
| SEQ ID | DNA coding | O- | Ps_CODM | From | artificial |
| NO: 873 | sequence of | demethylase | | | |
| SEQ ID | Amino acid | O- | Ps_CODM | From | Papaver |
| NO: 874 | sequence of | demethylase | | | somniferum |
| SEQ ID | DNA coding | N- | — | From | artificial |
| NO: 875 | sequence of | demethylase | | | |
| SEQ ID | Amino acid | N- | — | From | artificial |
| NO: 876 | sequence of | demethylase | | | |
| SEQ ID | DNA coding | UGT | KAF3968553 | From | artificial |
| NO: 877 | sequence of | | | | |
| SEQ ID | Amino acid | UGT | KAF3968553 | From | Castanea |
| NO: 878 | sequence of | | | | mollissima |
| SEQ ID | DNA coding | UGT | Qs72S_1 | From | artificial |
| NO: 879 | sequence of | | | | |
| SEQ ID | Amino acid | UGT | Qs72S_1 | From | Quercus suber |
| NO: 880 | sequence of | | | | |
| SEQ ID | DNA coding | UGT | XP_023875154 | From | artificial |
| NO: 881 | sequence of | | | | |
| SEQ ID | Amino acid | UGT | XP_023875154 | From | Quercus suber |
| NO: 882 | sequence of | | | | |
| SEQ ID | DNA coding | UGT | XP_023914549 | From | artificial |
| NO: 883 | sequence of | | | | |
| SEQ ID | Amino acid | UGT | XP_023914549 | From | Quercus suber |
| NO: 884 | sequence of | | | | |
| SEQ ID | DNA coding | UGT | KAF3968554 | From | artificial |
| NO: 885 | sequence of | | | | |
| SEQ ID | Amino acid | UGT | KAF3968554 | From | Castanea |
| NO: 886 | sequence of | | | | mollissima |
| SEQ ID | DNA coding | UGT | XP_023905565 | From | artificial |
| NO: 887 | sequence of | | | | |
| SEQ ID | Amino acid | UGT | XP_023905565 | From | Quercus suber |
| NO: 888 | sequence of | | | | |
| SEQ ID | DNA coding | UGT | XP_030967178 | From | artificial |
| NO: 889 | sequence of | | | | |
| SEQ ID | Amino acid | UGT | XP_030967178 | From | Quercus lobata |
| NO: 890 | sequence of | | | | |
| SEQ ID | DNA coding | UGT | XP_023876282 | From | artificial |
| NO: 891 | sequence of | | | | |
| SEQ ID | Amino acid | UGT | XP_023876282 | From | Quercus suber |
| NO: 892 | sequence of | | | | |
| SEQ ID | DNA coding | UGT | XP_023876189 | From | artificial |
| NO: 893 | sequence of | | | | |
| SEQ ID | Amino acid | UGT | XP_023876189 | From | Quercus suber |
| NO: 894 | sequence of | | | | |
| SEQ ID | DNA coding | UGT | XP_023923919 | From | artificial |
| NO: 895 | sequence of | | | | |
| SEQ ID | Amino acid | UGT | XP_023923919 | From | Quercus suber |
| NO: 896 | sequence of | | | | |
| SEQ ID | DNA coding | UGT | KAB1219588 | From | artificial |
| NO: 897 | sequence of | | | | |
| SEQ ID | Amino acid | UGT | KAB1219588 | From | Morella rubra |
| NO: 898 | sequence of | | | | |
| SEQ ID | DNA coding | glycosyl | ScUGP1 | From | Saccharomyces |
| NO: 899 | sequence of | donor | | | cerevisiae |
| SEQ ID | Amino acid | glycosyl | ScUGP1 | From | Saccharomyces |
| NO: 900 | sequence of | donor | | | cerevisiae |
| SEQ ID | DNA coding | transcription | PDR1 | From | Saccharomyces |
| NO: 901 | sequence of | factor | | | cerevisiae |
| SEQ ID | Amino acid | transcription | PDR1 | From | Saccharomyces |
| NO: 902 | sequence of | factor | | | cerevisiae |
| SEQ ID | DNA coding | transcription | PDR3 | From | Saccharomyces |
| NO: 903 | sequence of | factor | | | cerevisiae |
| SEQ ID | Amino acid | transcription | PDR3 | From | Saccharomyces |
| NO: 904 | sequence of | factor | | | cerevisiae |
| SEQ ID | DNA coding | transcription | YRR1 | From | Saccharomyces |
| NO: 905 | sequence of | factor | | | cerevisiae |
| SEQ ID | Amino acid | transcription | YRR1 | From | Saccharomyces |
| NO: 906 | sequence of | factor | | | cerevisiae |
| SEQ ID | DNA coding | transcription | PDR8 | From | Saccharomyces |
| NO: 907 | sequence of | factor | | | cerevisiae |
| SEQ ID | Amino acid | transcription | PDR8 | From | Saccharomyces |
| NO: 908 | sequence of | factor | | | cerevisiae |
| SEQ ID | DNA coding | efflux | ET60 | From | artificial |
| NO: 909 | sequence of | transporter | | | |
| SEQ ID | Amino acid | efflux | ET60 | From | Debaryomyces |
| NO: 910 | sequence of | transporter | | | fabryi |
| SEQ ID | DNA coding | efflux | ET71 | From | artificial |
| NO: 911 | sequence of | transporter | | | |
| SEQ ID | Amino acid | efflux | ET71 | From | Wickerhamomyces |
| NO: 912 | sequence of | transporter | | | ciferrii |
| SEQ ID | DNA coding | efflux | ET58 | From | artificial |
| NO: 913 | sequence of | transporter | | | |
| SEQ ID | Amino acid | efflux | ET58 | From | Cyberlindnera |
| NO: 914 | sequence of | transporter | | | fabianii |
| SEQ ID | DNA coding | efflux | PDR5 | From | Saccharomyces |
| NO: 915 | sequence of | transporter | | | cerevisiae |
| SEQ ID | Amino acid | efflux | PDR5 | From | Saccharomyces |
| NO: 916 | sequence of | transporter | | | cerevisiae |
| SEQ ID | DNA coding | efflux | ET161 | From | artificial |
| NO: 917 | sequence of | transporter | | | |
| SEQ ID | Amino acid | efflux | ET161 | From | Mus musculus |
| NO: 918 | sequence of | transporter | | | |
| SEQ ID | DNA coding | efflux | ET63 | From | artificial |
| NO: 919 | sequence of | transporter | | | |
| SEQ ID | Amino acid | efflux | ET63 | From | Ogataea philodendri |
| NO: 920 | sequence of | transporter | | | |
| SEQ ID | DNA coding | efflux | ET170 | From | artificial |
| NO: 921 | sequence of | transporter | | | |
| SEQ ID | Amino acid | efflux | ET170 | From | Capra hircus |
| NO: 922 | sequence of | transporter | | | |
| SEQ ID | DNA coding | efflux | ET97 | From | artificial |
| NO: 923 | sequence of | transporter | | | |
| SEQ ID | Amino acid | efflux | ET97 | From | Cyanobium sp. |
| NO: 924 | sequence of | transporter | | | NIES-981 |
| SEQ ID | DNA coding | efflux | ET82 | From | artificial |
| NO: 925 | sequence of | transporter | | | |
| SEQ ID | Amino acid | efflux | ET82 | From | Cyberlindnera |
| NO: 926 | sequence of | transporter | | | jadinii NRRL |
| | | | | | Y-1542 |
| SEQ ID | DNA coding | efflux | ET81 | From | artificial |
| NO: 927 | sequence of | transporter | | | |
| SEQ ID | Amino acid | efflux | ET81 | From | Wickerhamomyces |
| NO: 928 | sequence of | transporter | | | mucosus |
| SEQ ID | DNA coding | efflux | ET83 | From | artificial |
| NO: 929 | sequence of | transporter | | | |
| SEQ ID | Amino acid | efflux | ET83 | From | Cyberlindnera |
| NO: 930 | sequence of | transporter | | | americana |
| SEQ ID | DNA coding | efflux | ET72 | From | artificial |
| NO: 931 | sequence of | transporter | | | |
| SEQ ID | Amino acid | efflux | ET72 | From | Ogataea |
| NO: 932 | sequence of | transporter | | | philodendri |
| SEQ ID | DNA coding | efflux | ET47 | From | artificial |
| NO: 933 | sequence of | transporter | | | |
| SEQ ID | Amino acid | efflux | ET47 | From | artificial |
| NO: 934 | sequence of | transporter | | | |
| SEQ ID | DNA coding | efflux | ET120 | From | artificial |
| NO: 935 | sequence of | transporter | | | |
| SEQ ID | Amino acid | efflux | ET120 | From | Lachancea |
| NO: 936 | sequence of | transporter | | | mirantina |
| SEQ ID | DNA coding | efflux | ET212 | From | artificial |
| NO: 937 | sequence of | transporter | | | |
| SEQ ID | Amino acid | efflux | ET212 | From | [Candida] auris |
| NO: 938 | sequence of | transporter | | | |
| SEQ ID | DNA coding | efflux | ET208 | From | artificial |
| NO: 939 | sequence of | transporter | | | |
| SEQ ID | Amino acid | efflux | ET208 | From | Kuraishia capsulata |
| NO: 940 | sequence of | transporter | | | CBS 1993 |
| SEQ ID | DNA coding | efflux | ET193 | From | artificial |
| NO: 941 | sequence of | transporter | | | |
| SEQ ID | Amino acid | efflux | ET193 | From | Scheffersomyces |
| NO: 942 | sequence of | transporter | | | stipitis CBS 6054 |
| SEQ ID | DNA coding | efflux | ET171 | From | artificial |
| NO: 943 | sequence of | transporter | | | |
| SEQ ID | Amino acid | efflux | ET171 | From | Capra hircus |
| NO: 944 | sequence of | transporter | | | |
| SEQ ID | DNA coding | efflux | ET168 | From | artificial |
| NO: 945 | sequence of | transporter | | | |
| SEQ ID | Amino acid | efflux | ET168 | From | Ovis aries |
| NO: 946 | sequence of | transporter | | | |
| SEQ ID | DNA coding | efflux | ET165 | From | artificial |
| NO: 947 | sequence of | transporter | | | |
| SEQ ID | Amino acid | efflux | ET165 | From | Mus musculus |
| NO: 948 | sequence of | transporter | | | |
| SEQ ID | DNA coding | efflux | ET160 | From | artificial |
| NO: 949 | sequence of | transporter | | | |
| SEQ ID | Amino acid | efflux | ET160 | From | Homo sapiens |
| NO: 950 | sequence of | transporter | | | |
| SEQ ID | DNA coding | Jefflux | ET158 | From | artificial |
| NO: 951 | sequence of | transporter | | | |
| SEQ ID | Amino acid | efflux | ET158 | From | Homo sapiens |
| NO: 952 | sequence of | transporter | | | |
| SEQ ID | DNA coding | efflux | ET159 | From | artificial |
| NO: 953 | sequence of | transporter | | | |
| SEQ ID | Amino acid | efflux | ET159 | From | Homo sapiens |
| NO: 954 | sequence of | transporter | | | |
| SEQ ID | DNA coding | efflux | ET319 | From | artificial |
| NO: 955 | sequence of | transporter | | | |
| SEQ ID | Amino acid | efflux | ET319 | From | Candida |
| NO: 956 | sequence of | transporter | | | oxycetoniae |
| SEQ ID | DNA coding | efflux | ET320 | From | artificial |
| NO: 957 | sequence of | transporter | | | |
| SEQ ID | Amino acid | efflux | ET320 | From | Hyphopichia |
| NO: 958 | sequence of | transporter | | | burtonii |
| SEQ ID | DNA coding | efflux | ET328 | From | artificial |
| NO: 959 | sequence of | transporter | | | |
| SEQ ID | Amino acid | efflux | ET328 | From | Candida |
| NO: 960 | sequence of | transporter | | | duobushaemulonis |
| SEQ ID | DNA coding | efflux | ET329 | From | artificial |
| NO: 961 | sequence | transporter | | | |
| SEQ ID | Amino acid | efflux | ET329 | From | Candida haemuloni |
| NO: 962 | sequence of | transporter | | | |
| SEQ ID | DNA coding | efflux | ET331 | From | artificial |
| NO: 963 | sequence | transporter | | | |
| SEQ ID | Amino acid | efflux | ET331 | From | Clavispora |
| NO: 964 | sequence of | transporter | | | lusitaniae |
| SEQ ID | DNA coding | efflux | ET332 | From | artificial |
| NO: 965 | sequence of | transporter | | | |
| SEQ ID | Amino acid | efflux | ET332 | From | Candida auris |
| NO: 966 | sequence of | transporter | | | |
| SEQ ID | DNA coding | efflux | ET325 | From | artificial |
| NO: 967 | sequence of | transporter | | | |
| SEQ ID | Amino acid | efflux | ET325 | From | Candida margitis |
| NO: 968 | sequence of | transporter | | | |
| SEQ ID | DNA coding | efflux | ET322 | From | artificial |
| NO: 969 | sequence of | transporter | | | |
| SEQ ID | Amino acid | efflux | ET322 | From | Candida theae |
| NO: 970 | sequence of | transporter | | | |
| SEQ ID | DNA coding | efflux | ET294 | From | artificial |
| NO: 971 | sequence of | transporter | | | |
| SEQ ID | Amino acid | efflux | ET294 | From | Lachancea |
| NO: 972 | sequence of | transporter | | | quebecensis |
| SEQ ID | DNA coding | efflux | ET283 | From | artificial |
| NO: 973 | sequence of | transporter | | | |
| SEQ ID | Amino acid | efflux | ET283 | From | Zygosaccharomyces |
| NO: 974 | sequence of | transporter | | | rouxii |
| SEQ ID | DNA coding | efflux | ET306 | From | artificial |
| NO: 975 | sequence o | transporter | | | |
| SEQ ID | Amino acid | efflux | ET306 | From | Cyberlindnera |
| NO: 976 | sequence of | transporter | | | fabianii |
| SEQ ID | DNA coding | efflux | ET305 | From | artificial |
| NO: 977 | sequence of | transporter | | | |
| SEQ ID | Amino acid | efflux | ET305 | | Wickerhamomyces |
| NO: 978 | sequence of | transporter | | | anomalus |
| SEQ ID | DNA coding | efflux | ET291 | From | artificial |
| NO: 979 | sequence of | transporter | | | |
| SEQ ID | Amino acid | efflux | ET291 | From | Lachancea |
| NO: 980 | sequence of | transporter | | | nothofagi |
| SEQ ID | DNA coding | efflux | ET281 | From | artificial |
| NO: 981 | sequence of | transporter | | | |
| SEQ ID | Amino acid | efflux | ET281 | From | Zygosaccharomyces |
| NO: 982 | sequence of | transporter | | | rouxii |
| SEQ ID | DNA coding | efflux | ET264 | From | artificial |
| NO: 983 | sequence of | transporter | | | |
| SEQ ID | Amino acid | efflux | ET264 | From | Naumovozyma |
| NO: 984 | sequence of | transporter | | | dairenensis |
| SEQ ID | DNA coding | efflux | ET304 | From | artificial |
| NO: 985 | sequence of | transporter | | | |
| SEQ ID | Amino acid | efflux | ET304 | From | Wickerhamomyces |
| NO: 986 | sequence of | transporter | | | ciferrii |
| SEQ ID | DNA coding | efflux | ET290 | From | artificial |
| NO: 987 | sequence of | transporter | | | |
| SEQ ID | Amino acid | efflux | ET290 | From | Lachancea |
| NO: 988 | sequence of | transporter | | | dasiensis |
| SEQ ID | DNA coding | efflux | ET289 | From | artificial |
| NO: 989 | sequence of | transporter | | | |
| SEQ ID | Amino acid | efflux | ET289 | From | Lachancea |
| NO: 990 | sequence of | transporter | | | meyersii |
| SEQ ID | DNA coding | efflux | ET316 | From | artificial |
| NO: 991 | sequence of | transporter | | | |
| SEQ ID | Amino acid | efflux | ET316 | From | Hanseniaspora |
| NO: 992 | sequence of | transporter | | | uvarum |
| SEQ ID | DNA coding | efflux | ET287 | From | artificial |
| NO: 993 | sequence of | transporter | | | |
| SEQ ID | Amino acid | efflux | ET287 | From | Lachancea |
| NO: 994 | sequence of | transporter | | | lanzarotensis |
| SEQ ID | DNA coding | efflux | ET273 | From | artificial |
| NO: 995 | sequence of | transporter | | | |
| SEQ ID | Amino acid | efflux | ET273 | From | Torulaspora |
| NO: 996 | sequence of | transporter | | | globosa |
| SEQ ID | DNA coding | efflux | ET312 | From | artificial |
| NO: 997 | sequence of | transporter | | | |
| SEQ ID | Amino acid | efflux | ET312 | From | Debaryomyces |
| NO: 998 | sequence of | transporter | | | hansenii |
| SEQ ID | DNA coding | efflux | ET300 | From | artificial |
| NO: 999 | sequence of | transporter | | | |
| SEQ ID | Amino acid: | efflux | ET300 | From | Kluyveromyces |
| NO: 1000 | sequence of | transporter | | | marxianus |
| SEQ ID | DNA coding | efflux | ET286 | From | artificial |
| NO: 1001 | sequence of | transporter | | | |
| SEQ ID | Amino acid | efflux | ET286 | From | Zygosaccharomyces |
| NO: 1002 | sequence of | transporter | | | bailii |
| SEQ ID | DNA coding | efflux | ET272 | From | artificial |
| NO: 1003 | sequence of | transporter | | | |
| SEQ ID | Amino acid | efflux | ET272 | From | Kazachstania |
| NO: 1004 | sequence of | transporter | | | africana |
| SEQ ID | DNA coding | efflux | ET311 | From | artificial |
| NO: 1005 | sequence of | transporter | | | |
| SEQ ID | Amino acid | efflux | ET311 | From | Kuraishia capsulata |
| NO: 1006 | sequence of | transporter | | | |
| SEQ ID | DNA coding | efflux | ET307 | From | artificial |
| NO: 1007 | sequence of | transporter | | | |
| SEQ ID | Amino acid | efflux | ET307 | From | Cyberlindnera |
| NO: 1008 | sequence of | transporter | | | americana |
| SEQ ID | DNA coding | efflux | ET295 | From | artificial |
| NO: 1009 | sequence of | transporter | | | |
| SEQ ID | Amino acid | efflux | ET295 | From | Tetrapisispora |
| NO: 1010 | sequence of | transporter | | | blattae |
| SEQ ID | DNA coding | efflux | ET282 | From | artificial |
| NO: 1011 | sequence of | transporter | | | |
| SEQ ID | Amino acid | efflux | ET282 | From | Zygosaccharomyces |
| NO: 1012 | sequence of | transporter | | | bailii |
| SEQ ID | DNA coding | efflux | ET270 | From | artificial |
| NO: 1013 | sequence of | transporter | | | |
| SEQ ID | Amino acid | efflux | ET270 | From | Hanseniaspora |
| NO: 1014 | sequence of | transporter | | | guilliermondii |
| SEQ ID | DNA coding | efflux | ET268 | From | artificial |
| NO: 1015 | sequence of | transporter | | | |
| SEQ ID | Amino acid | efflux | ET268 | From | Kazachstania |
| NO: 1016 | sequence of | transporter | | | saulgeensis |
| SEQ ID | DNA coding | efflux | ET252 | From | artificial |
| NO: 1017 | sequence of | transporter | | | |
| SEQ ID | Amino acid | efflux | ET252 | From | Lichtheimia ramosa |
| NO: 1018 | sequence of | transporter | | | |
| SEQ ID | DNA coding | efflux | ET303 | From | artificial |
| NO: 1019 | sequence of | transporter | | | |
| SEQ ID | Amino acid | efflux | ET303 | From | Wickerhamomyces |
| NO: 1020 | sequence of | transporter | | | mucosus |
| SEQ ID | DNA coding | efflux | ET274 | From | artificial |
| NO: 1021 | sequence of | transporter | | | |
| SEQ ID | Amino acid | efflux | ET274 | From | Tetrapisispora |
| NO: 1022 | sequence of | transporter | | | phaffii CBS 4417 |
| SEQ ID | DNA coding | efflux | ET301 | From | artificial |
| NO: 1023 | sequence of | transporter | | | |
| SEQ ID | Amino acid | efflux | ET301 | From | Kazachstania |
| NO: 1024 | sequence of | transporter | | | exigua |
| SEQ ID | DNA coding | efflux | ET265 | From | artificial |
| NO: 1025 | sequence of | transporter | | | |
| SEQ ID | Amino acid | efflux | ET265 | From | Kazachstania |
| NO: 1026 | sequence of | transporter | | | africana CBS 2517 |
| SEQ ID | DNA coding | efflux | ET299 | From | artificial |
| NO: 1027 | sequence of | transporter | | | |
| SEQ ID | Amino acid | efflux | ET299 | From | Kluyveromyces |
| NO: 1028 | sequence of | transporter | | | dobzhanskii |
| SEQ ID | DNA coding | efflux | ET293 | From | artificial |
| NO: 1029 | sequence of | transporter | | | |
| SEQ ID | Amino acid | efflux | ET293 | From | Lachancea |
| NO: 1030 | sequence of | transporter | | | mirantina |
| SEQ ID | DNA coding | efflux | ET196 | From | artificial |
| NO: 1031 | sequence of | transporter | | | |
| SEQ ID | Amino acid | efflux | ET196 | From | Clavispora |
| NO: 1032 | sequence of | transporter | | | lusitaniae |
| SEQ ID | DNA coding | efflux | ET202 | From | artificial |
| NO: 1033 | sequence of | transporter | | | |
| SEQ ID | Amino acid | efflux | ET202 | From | Pachysolen |
| NO: 1034 | sequence of | transporter | | | tannophilus |
| SEQ ID | DNA coding | efflux | ET306 | From | artificial |
| NO: 1035 | sequence of | transporter | | | |
| SEQ ID | Amino acid | efflux | ET306 | From | Cyberlindnera |
| NO: 1036 | sequence of | transporter | | | fabianii |
| SEQ ID | DNA coding | efflux | ET316 | From | artificial |
| NO: 1037 | sequence of | transporter | | | |
| SEQ ID | Amino acid | efflux | ET316 | From | Hanseniaspora |
| NO: 1038 | sequence of | transporter | | | uvarum |
| SEQ ID | DNA coding | efflux | ET321 | From | artificial |
| NO: 1039 | sequence of | transporter | | | |
| SEQ ID | Amino acid | efflux | ET321 | From | Candida theae |
| NO: 1040 | sequence of | transporter | | | |
| SEQ ID | Amino acid | Walker A | consensus | From | artificial |
| NO: 1041 | sequence of | motif variant | | | |
| | | 1 | | | |
| SEQ ID | Amino acid | Walker A | consensus | From | artificial |
| NO: 1042 | sequence of | motif variant | | | |
| | | 2 | | | |
| SEQ ID | Amino acid | Walker A | consensus | From | artificial |
| NO: 1043 | sequence of | motif variant | | | |
| | | 3 | | | |
| SEQ ID | Amino acid | Walker A | consensus | From | artificial |
| NO: 1044 | sequence of | motif variant | | | |
| | | 4 | | | |
| SEQ ID | Amino acid | Walker A | consensus | From | artificial |
| NO: 1045 | sequence of | motif variant | | | |
| | | 5 | | | |
| SEQ ID | Amino acid | Walker A | consensus | From | artificial |
| NO: 1046 | sequence of | motif variant | | | |
| | | 6 | | | |
| SEQ ID | Amino acid | Walker A | consensus | From | artificial |
| NO: 1047 | sequence of | motif variant | | | |
| | | 7 | | | |
| SEQ ID | Amino acid | Walker A | consensus | From | artificial |
| NO: 1048 | sequence of | motif variant | | | |
| | | 8 | | | |
| SEQ ID | Amino acid | Walker A | consensus | From: | artificial |
| NO: 1049 | sequence of | motif variant | | | |
| | | 9 | | | |
| SEQ ID | Amino acid | Walker A | consensus | From | artificial |
| NO: 1050 | sequence of | motif variant | | | |
| | | 10 | | | |
| SEQ ID | Amino acid | Walker A | consensus | From | artificial |
| NO: 1051 | sequence of | motif variant | | | |
| | | 11 | | | |
| SEQ ID | Amino acid | Walker A | consensus | From | artificial |
| NO: 1052 | sequence of | motif variant | | | |
| | | 12 | | | |
| SEQ ID | Amino acid | Walker A | consensus | From | artificial |
| NO: 1053 | sequence of | motif variant | | | |
| | | 13 | | | |
| SEQ ID | Amino acid | Walker A | consensus | From | artificial |
| NO: 1054 | sequence of | motif variant | | | |
| | | 14 | | | |
| SEQ ID | Amino acid | Walker A | consensus | From | artificial |
| NO: 1055 | sequence of | motif variant | | | |
| | | 15 | | | |
| SEQ ID | Amino acid | Walker A | consensus | From | artificial |
| NO: 1056 | sequence of | motif | | | |
| | | consensus | | | |
| SEQ ID | Amino acid | consensus | consensus | From | artificial |
| NO: 1057 | sequence of | Walker | | | |
| | | motifs | | | |
| SEQ ID | Amino acid | ABC efflux | consensus | From | artificial |
| NO: 1058 | sequence of | transporter | | | |
| | | consensus | | | |
| SEQ ID | Amino acid | ABC efflux | consensus | From | artificial |
| NO: 1059 | sequence of | transporter | | | |
| | | consensus | | | |
| SEQ ID | Amino acid | ABCC efflux | consensus | From | artificial |
| NO: 1060 | sequence of | transporter | | | |
| | | consensus | | | |
| SEQ ID | Amino acid | ABCG efflux | consensus | From | artificial |
| NO: 1061 | sequence of | transporter | | | |
| | | consensus | | | |
| | | Walker A | | | |
| SEQ ID | Amino acid | ABCG efflux | consensus | From | artificial |
| NO: 1062 | sequence of | transporter | | | |
| | | consensus | | | |
| | | Walker A | | | |
| SEQ ID | Amino acid | ABCG efflux | consensus | From | artificial |
| NO: 1063 | sequence of | transporter | | | |
| | | consensus | | | |
| | | linker | | | |
| SEQ ID | Amino acid | ABCG efflux | consensus | From | artificial |
| NO: 1064 | sequence of | transporter | | | |
| | | consensus | | | |
| | | linker | | | |
| SEQ ID | Amino acid | ABCG efflux | consensus | From | artificial |
| NO: 1065 | sequence of | transporter | | | |
| | | consensus | | | |
| | | Walker B | | | |
| SEQ ID | Amino acid | ABCG efflux | consensus | From | artificial |
| NO: 1066 | sequence of | transporter | | | |
| | | consensus | | | |
| | | Walker B | | | |
| SEQ ID | Amino acid | ABCG efflux | consensus | From | artificial |
| NO: 1067 | sequence of | transporter | | | |
| | | consensus | | | |

The above disclosed efflux transporters have the following sequences as shown in Table 2:

| | |
|---|---|
| SEQ ID | ATGGTCGAAAACAAGGCCAACAAGTTGGACTCTTCATCTTTGGAAGATAACGGTTTGGAAAG |
| NO: 909 | ACAACAGAGGTTGTTGTCATTTTTGTGGCCAAAAACTGTTCCACCATTGCCAAATGAAGATG |
| | AGAGATTGCTATACGGTGAAAAGAGAGCTGGTATTTTCTCTAAGGCTTTCTTCTGGTGGATG |
| | ATCCCAGTTATGAATCCAGGTTATATGAGAACCTTGCAGCCAGAAGATTTGTTCACTTTGACC |
| | GATGATATCTCCGTCGAACAAATGTCTGCTAGGTTTAACAAGCTGTTCAAGAAGAAGGTTGA |
| | TAAGGCTAAGAGAAAGCACATCATCCAAAAGTTCAAGAACAGAAACGAAAAGGTCGAGATC |
| | TCCGATATTGATCAGTACAAGGATGACTTGGAAGATTTCACTCCACCACAATTTTTGCCATGG |
| | TTCGTTATTATCGAAACCTTCAAGTGGGAATACTTCGCTGCTGTTATTTTCTTGGCTTTGATGT |
| | ACGGTACGAGTTCTTGTATTGCTCTGGTTACCAAAGAGTTGATCAAGTACGTTGAGTACAAA |
| | GCCGTTGGTGTTGAATTAGGTATTGGTAAAGGTTTGGGTTACGCTTTTGGTACTGTTGGTAT |
| | GGTTGTTTTCACTGGTTTTATGGGTAACCACTACTTCTACAGAGCTATGTTGATTGGTGCTAA |
| | GACTAAGGCCGTTCTGATTAAGTCTATCTTGGACAAGTCCTTCATCCTGTCTCCAAAGTCTAA |
| | GTTGAATTTCCCACATGCCAAGATCACCTCTATGATGTCTACTGATACTGCCAGAATTGACTT |
| | AGGTTTGGGATTGCAACCTCTGCTGTTGATTATTCCAATTCCAATCATCGTTTCGATCGCCATC |
| | TTGATCGTTAACATTGGTGTTTCTGCTTTGACTGGTATTGCCGTTATCATCTTGGTTTTGGTTT |
| | TGATTATGGGTGTCGGCTACTTCTTGTTCAAGTTTAGAAAGAAGGCTAACATCTCCACCGACC |
| | AAAGAATTTCTTCTATCAGAGAAGTCCTGTACAACCTGAAGATCATCAAGTTTTACTCTTGGG |
| | AATCCGCCTACTTGAAGAAGATTTCTGGTATTAGGAACGAAGAGACTAAGTGGATCTTGAGA |
| | ATGCAAGTCTTGAGGAACTTGATTATCTCCATTGCCATCTCCGTTAACTTGATCTGTTCTATGG |
| | TTTCCTTCTTGGTCTTGTACGCCATTGATTCTGATAGACATGATCCAGCTTCTATCTTCTCTTCT |
| | TTGACCTTGTTCGGTATCTTGTCCGAACAAGTTATTATGTTGCCATTGGCTTTGGCTACTACTA |
| | CTGATGCTCATGTTGGTTTACAAAGAGTCGGTCAATTTTTGGCCTCTGAAGAATCTGATCAAA |
| | CCTACAGAAAGATTGAAGCTTCTGGTAAGACTTTGGGTCGTATGCAAGAAAACAACATTGCC |
| | GTTGAAGTTAACAACGCCACTTTCATTTGGGAAACCTTCGATGTTTCTGATGAGGACTCTAAA |
| | ATCTCCGACGAAAACTCTGATGAGTCCAAGAATTCCTCTACTACCAACTCTACTTCCGAAAGA |
| | AACTTGGATGAAGAGGATAAGGATAACGAAACTCCATTCAAGGGTTTGATCGATGTCAACTT |
| | GACTGTTAACAAGGGTGAATTCGTTGTTATCACCGGTGTTATTGGTTCGGGTAAATCATCTTT |
| | GTTGTCCGCTATTTCTGGTTTGATGACTAGAACTTCCGGTGAAGTTAATGTTTGCGGTTCTTT |
| | GATTTCTTGTGGTGAACCATGGATTCAGAACGAGACTTTCAAAGAGAACATTTTGTTCGGTTC |
| | CGATTTCGACCCAGACTTCTACAAAGAAGTTGTTCACGCTTGTTCCTTGGAATCCGATATGGA |
| | AATTTTGCCAGCTGGTGATAAGACCGAAATTGGTGAAAGAGGTATTACTTTGTCTGGTGGTC |
| | AAAAGGCTAGATTGAATTTGGCTAGAGCTGTTTACACCAACAAGGACATTATCTTGTTGGAT |
| | GACGTTTTGTCAGCTGTTGATGCTAGAGTCGGTAAACATATTATGAACAACTGCATCTTGGG |
| | CACCTTGTCCTCTAAAACTAGAATTTTGGCTACCCACCAGTTGTCTTTGATTGGTTCTGCTGAT |
| | AAGGTTATCTTCATGAACGGTGATGGTTCTTTGGAAATCGGTAAGTTCGATGAATTGATCCA |
| | GAACTCTTCTGGTTTCAAGGACTTGATGTCTTTGAATGCTCAAGAGGTTGTCAGAGATGTTAC |
| | CAACAATGTTGAAAACGATTCCAAGTTTGCTGGTGTTGAGGACGAAAAACAGTACATCGAA |
| | GAACAGTTGATGAGAAGAACTACCACCACCTCTTATATTGAGGATGAGAAATCTGGTAGAAA |
| | CGGCGTTAATTTGGATAGAATTGATGACGGCAAGCTATTCTTGGCTGAAGAAAGAGCTGTTA |
| | ACCGTATCGAATTCAAGGTCTACAAGAATTACGTCAAGTACGGTTCTGGTATCTTCTCCTCAT |
| | TCTGCATCATTTTCTTGTTCTTGTTGTTCACTGTCTTGGCTACTTACTTCGAGTTGTTTACTAAT |
| | ACCTGGTTGTCCTTCTGGACCTCTAAAAAGTTTCCAGGTAGATTGGACAACTTCTACATTGGC |
| | TTGTACGTTACTTTTACCTTCTTGGCCTTTATCTTCCTGACCTTGGAATTTTTCGTTTTGGCTTA |
| | CGTTACTACCATTGCCTCTAGAACTTTGAATTTGATGGCCGTCAAGAAGATCTTGTTTGTCCC |
| | AATGTCTTTCATGGATACAACTCCAATGGGTAGAATCTTCAACAGATTCACTAAGGATACCGA |
| | TGCTTTGGACAACGAAATCGTTGAACAATTGACCGTCCTGTTCTACTTCATTGCTAACATTAC |
| | CGGTGTTCTGATTTTGTGCATTTGTTACTTACCCTGGTTCGCTATTGCTGTTCCTCCTTTGTTGT |
| | TTTTGTTCGTTGCTATTGCTAACTACTACCAGGCTTCTGCTAGAGAAATCAAGAGATTGGAAG |
| | CAGTTCAGAGATCCTTCGTTTACGACAATTTCAACGAAACCTTGTCTGGTATGGGTACTATCG |
| | TTGCTTACAAATCTAAGCACAGGTTCCTAAACAAAAACTCCTTCCTGATCGACAAAATGAACG |
| | AAGCTTACTACTTGACTATTGCCAATCAGAGATGGCTGACCATTTCTTTGGATATGGTTGGTG |
| | CTGTTTTCGTCTTATTGGTTGCTATGTTGTGCGTTAACAGAGTGTTCCACATTAACTCATCATC |
| | CGTTGGTTTGCTAATGTCCTACATCTTGCAAATCGTTGGCCAATTGTCTTTCTTGCTGAAAACT |
| | TTGACCCAAGTCGAAAACGAAATGAACTCCGTTGAAAGAATTTGCCATTACGCTTTCGATTTG |
| | CCTGAAGAAGCTCCATACGTTATCACTGAAAATTCTCCACCACCATCTTGGCCAGAAAAAGGT |
| | CAAATTTCTTTTAACCATGCCTCCATGGCTTACAGACCAGAATTACCATTGGTCTTGAAGGAT |
| | TTGGATGTCAACATTAAGCCCATGGAAAAGATAGGTGTTTGTGGTAGAACTGGTGCTGGTA |
| | AGTCATCTATTATGATGGCCTTGTACAGATTGGTCGAATTGAATTCTGGTTCCGTCGAAATTG |
| | ATGGTATCGATATTTCTACCTTGGGCCTGAACAATTTGAGATCCAGATTGTCCATTATTCCAC |
| | AGGATCCAATTTTGTTCTCCGGTACTATTAGAACTAACTTGGATCCATTCGATGAGTACACTG |
| | ATACAGAATTGTGGGATGCTTTGAAGAGATCCGGTTTGATTGATGAGAGCAAGATCTCTTCT |
| | GTTCAATCCCAAGATCCAAAGTCCGAAGATTTGAACAAGTTCCATTTGTTCAAGCAGGTCCAA |
| | GAAAACGGTACAAACTTTTCATTGGGTGAGAGACAATTGATTGCTTTCGCTAGAGCTTTGGT |
| | CAAGAGAACTAAGATCTTGATTTTGGACGAAGCTACCTCTTCCGTTGATTACGAAACTGATAA |
| | CAAGATTCAAAAGACCATCCTGAAAGAGTTCGGTACTTGCACCATTTTGTGTATTGCCCATAG |
| | ACTGAAAACCATCATCAACTACGATAGGATTTTGGTCTTGGAAAAGGGTGAAGTCAAAGAAT |
| | TCGACACTCCATGGAATCTGTTCAACACTAAGGACTCCATTTTCGAACAGATGTGCAGAAAAT |
| | CCAAGATTACTTCCGATGATTTCACCATCAAGACCATCTGA |
| SEQ ID | MVENKANKLDSSSLEDNGLERQQRLLSFLWPKTVPPLPNEDERLLYGEKRAGIFSKAFFWWMIP |
| NO: 910 | VMNPGYMRTLQPEDLFTLTDDISVEQMSARFNKLFKKKVDKAKRKHIIQKFKNRNEKVEISDIDQ |
| | YKDDLEDFTPPQFLPWFVIIETFKWEYFAAVIFLALMYGTSSCIALVTKELIKYVEYKAVGVELGIGK |
| | GLGYAFGTVGMVVFTGFMGNHYFYRAMLIGAKTKAVLIKSILDKSFILSPKSKLNFPHAKITSMM |
| | STDTARIDLGLGLQPLLLIIPIPIIVSIAILIVNIGVSALTGIAVIILVLVLIMGVGYFLFKFRKKANIS |
| | TDQRISSIREVLYNLKIIKFYSWESAYLKKISGIRNEETKWILRMQVLRNLIISIAISVNLICSMVSFLV |
| | LYAIDSDRHDPASIFSSLTLFGILSEQVIMLPLALATTTDAHVGLQRVGQFLASEESDQTYRKIEASGKTL |
| | GRMQENNIAVEVNNATFIWETFDVSDEDSKISDENSDESKNSSTTNSTSERNLDEEDKDNETPFK |
| | GLIDVNLTVNKGEFVVITGVIGSGKSSLLSAISGLMTRTSGEVNVCGSLISCGEPWIQNETFKENILF |
| | GSDFDPDFYKEVVHACSLESDMEILPAGDKTEIGERGITLSGGQKARLNLARAVYTNKDIILLDDVL |
| | SAVDARVGKHIMNNCILGTLSSKTRILATHQLSLIGSADKVIFMNGDGSLEIGKFDELIQNSSGFKD |
| | LMSLNAQEVVRDVTNNVENDSKFAGVEDEKQYIEEQLMRRTTTTSYIEDEKSGRNGVNLDRIDD |
| | GKLFLAEERAVNRIEFKVYKNYVKYGSGIFSSFCIIFLFLLFTVLATYFELFTNTWLSFWTSKKFPGRL |
| | DNFYIGLYVTFTFLAFIFLTLEFFVLAYVTTIASRTLNLMAVKKILFVPMSFMDTTPMGRIFNRFTKD |
| | TDALDNEIVEQLTVLFYFIANITGVLILCICYLPWFAIAVPPLLFLFVAIANYYQASAREIKRLEAVQRS |
| | FVYDNFNETLSGMGTIVAYKSKHRFLNKNSFLIDKMNEAYYLTIANQRWLTISLDMVGAVFVLLV |
| | AMLCVNRVFHINSSSVGLLMSYILQIVGQLSFLLKTLTQVENEMNSVERICHYAFDLPEEAPYVITE |
| | NSPPPSWPEKGQISFNHASMAYRPELPLVLKDLDVNIKPMEKIGVCGRTGAGKSSIMMALYRLV |
| | ELNSGSVEIDGIDISTLGLNNLRSRLSIIPQDPILFSGTIRTNLDPFDEYTDTELWDALKRSGLIDESKI |
| | SSVQSQDPKSEDLNKFHLFKQVQENGTNFSLGERQLIAFARALVKRTKILILDEATSSVDYETDNKI |
| | QKTILKEFGTCTILCIAHRLKTIINYDRILVLEKGEVKEFDTPWNLFNTKDSIFEQMCRKSKITSDDFTI |
| | KTI |
| SEQ ID | ATGTCCTCCAACACCTCCATGAAGGATGAAGATGATTACCAAGACTTGGAAAAGTCCAACGC |
| NO: 911 | TCATATTCAACAACCTAGACCAGTCAAGAGATTATTGACTCCTTTGTTGACTAAGTACGTCCC |
| | ACCAATTCCACAAGAATCTGAAAGAACTAGATTCCCATTCTACCACACCAACATTTTCTCTAA |
| | GGCTTTGTTTGCTTGGTTGTTGCCTTTGTTGTTTAAGGGTTACAAGAGAACCTTGCAACAAGA |
| | GGATTTGTGGAAGTTGGATGAACATACCTCTATCGATCACGTCTACATCAAGTTTGAAAAGC |
| | ACTTGAACGAAGAGTGGTTGAAGTTCGATGCTAAACATGATCCAATCAAGAACCCAGATGCT |
| | TTTCCTAGATTCGCTATTTTCATGGCTTTGATGAAGACCTTCAAGTACGAGTATACTGTTGCTA |
| | TCGTTACCAAGATTATCTCCAATGCTTTGTCCGCTTTTACCCCATTGGTTTCCAAAAAGTTGAT |
| | CTCCTTCATCTCCGAAAAAGCTTTGGTTCCAGATACGCCTATTAACAAAGGTATTGGTTACGC |
| | TTTCGGTATCACCTTCATGTTGATGTTCTCTGCCATCTTTATGAACCAGTCCTTGTTGCATTCTA |
| | AGTTCGTTGGTGGTCATTCCAGAACTATTTTGACCAAGGCTTTGATCCAAAAGTCCTTGATTG |
| | CTAACGCTGAAACCAGATTTCATTACCCATCCGGTAGAATCATCTCATTCATGTCTGCTGACTT |
| | GCAAAGAATCGACGAATCTTTGTTTGAATTGCCAACTGGTTTCACTACCTTGGAGCCAATTAT |
| | CATTGCTATTGTCTTGCTGATCGTTAACATTGGTGTTTCTGCTTTGGCTGGTATTGCTATCTTT |
| | TTCTTGACCTTGATCTTGATGGGTGTTCCAGCTGGTTCTTTGTTCAAAATTAGAGAAGCTGCT |
| | AACGTGTTCACCGATCAACGTGTTGGTAAAATGAGAGAAGTCATCCAGTCTATGAAGATGAT |
| | CAAGTTCTACTCTTGGGAAGATGCCTACGAAAACTTGATTACCGGTATCAGGTCTAAAGAGT |
| | CCTCTTTGGTTTTGAAGTTCCAGTTGACCATCAACGTCATGATTACTATTGCTATTAACGCCTC |
| | CTCCATTACTTCTATGGGTGCATTTTTGGTCTTGTACGCTGTTAAGTCTCATGGTAATCCAGCT |
| | AACGTTTTCTCCTCTTTGTCTTTGTTCGGTATCTTGTCCCAACAAGTTATCGAATTGCCTATGG |
| | TTTTTAGCTCTGCTGCTGAAGGTTTGTTGTCCTTGGATAGAATTACGAAGTACTTGAGATCTC |
| | CAGTTGAAACCTTTGACGTCGAAAACTTCTACGACTCCGAATTGATTAAGAACGACGAAATT |
| | GCCGTCCAAATCGAAAATGGTGAATTTGAATGGGAGCTGTTCACTGAGATCAAAGAAGATG |
| | ACGAAGAAACGAAAAAGCAGAAGAAGAAGGACGAAAAGCAGCGTAAGAAAGAGCTGAAA |
| | AAGTCTCAAGGTGGTAATGGTTGGTTCAACAAAAAGACTAAGACCACCGAAGATTCCTCCAA |
| | TGACGAAATCAACAAAGAATCCTCTACCGACGAAGATAACACTAATCAACAGAAGCCATTCA |
| | AGCTGTCCAACATCAACTTAAAGATTTCCAAGGGTGAATTCATCGTTGTTACTGGTCCAATTG |
| | GTTCCGGTAAATCTTCTTTATTGTCCGCTATTTCTGCCTTCATGACTAAGACTGATGGTAAGAT |
| | TGCTATCAACGGCTCTAATTTGTTGTGTGGTGCTCCATGGGTTCAAAACACTACTATTCGTGA |
| | AAACGTCTTGTTCGGTTCCAAGTTCGATCATGTTAAGTACAAGAAGGTTTTGGAGGTCTGCTC |
| | TTTGGAACATGATTTGAAGTCTTTGTTGGCTGGTGATATGACCGAAATTGGTGAAAGAGGTG |
| | TTACTTTGTCTGGTGGTCAAAAGGCTAGAGTTAATTTGGCTAGAGCTGTTTACGCTGACAAA |
| | GAAGTTTACTTGTTCGACGATATTTTGTCCGCCGTTGATGCTAATGTTGGTAAGAACATTACC |
| | GAGAACTGTTTGTTGGGTCTGTTGTCCTCTAAGACCATTATTATCGCTACCCATCAGCTGTCCT |
| | TAATTTCTAAAGCTGATAGGGTCGTTTTCTTGAACGGTGATGGTTTGATAGATGTCGGTACTG |
| | AATCTGAATTGAGGTCCAAGAACAAGGATTTTGTCAAGTTGATGGAGTACAACAAAGAGTT |
| | GGAACAAAAGAACACCGACGACGAAGAACAAATCAACGATAAGATTACTAAGGTCACCTCC |
| | ATTGCTGATAAGCCATCTGGTCCTATTGATGGTACTTTATTCGGTGAAGAAGAAAGGGCCTT |
| | TGATTCCATTCCATTGTCCTTGTACAAACAATACGTTAAGGCTGGTCAAGGTATGTTTGGTTT |
| | TACTGCTTTCCCATTGACCATTATCTGCATCATCTTGTCCGTTTTCGTCAACTTGTTCACTAACG |
| | TTTGGTTGTCTTTTTGGGTTGCCCAAAAGTTCAAGAACTTGTCTAATGGTCAGTACATCGGCT |
| | TGTACGTTATGTTTACAGTTCTGTCTGTTCTGTTCGTTGTCGTTGAATTGGCTATTATGGGTTA |
| | CGTTTTTACCGAAGCTTCTAAGACGTTGAACTTGAAGGCTATGCAAAAGGTTTTACACTCCCC |
| | AATGTCTTTCATTGATACAACTCCAGTCGGTAGGATCATCAACAGATTTTCTAAGGACACCAA |
| | CAGCTTGGATAACGAAATAGGTATGCAGCTGAAGTTGTTCTTGCATTTCTCTTCCACCATCAT |
| | CGGCATTATCATTTTGGCCATTATCTACTTGCCATGGTTCGCTATTGCTGTTCCATTTTTGGCT |
| | ATTTTCTTTTTGTGCGCCACCAACTTCTATCAAGCTTCTTCTAGAGAAGTGAAGAGATTGGAA |
| | GCCATTAACAGATCCTTCGTCTACAACAATTTCAACGAAGTCATGAACGGTATGAACACCATT |
| | AAGGCTTATGGTGCTGGTGAAAGGTTCATCAGAAAGAATGATATTTTCGGCGACCAATTGAA |
| | CGAGGTTTACTTCGTTGTTGTTTCTAACCAGAGATGGATTGCCGTTAACTTGGATATTATGGC |
| | TACTGCCGTTGTTTTCATTGTCGCTATGTTGTCTGTTACCGGTCAATTTTCAATCAACGCTTCA |
| | TCTGTTGGTCTGCTAACCTACTACATGATTGAATTGTCCCAGATGTTGAGCTTCTTGATGCAA |
| | ACTTACTCCGAAGTCGAAAACGAAATGAACTCTGTTGAAAGAGTTTGCCATTACGCCAACGA |
| | TTTGGAACAAGAATCAGCTTACAGAACTTTGGACTATCAGCCAAGACCAACTTGGCCTGAAG |
| | AAGGTGGTATTAAGTTTGACAACTTGTCCTTGAGGTACAGAGATGGTTTACCATTGGTCTTG |
| | AAGAACCTGTCCATAGATATCAAAGGTGGTGAAAAGATTGGTATCTGCGGTAGAACAGGTG |
| | CTGGTAAATCTAGTTTGATGATAGCCTTGTACAGAATTGCTGAATTCGCTGAAGGTGGCATTT |
| | TTATCGATGGTACTGATATTTCTAAGCTGGGCTTGCACGATTTGAGATCCAAGTTGTCTATTA |
| | TCCCACAAGATCCAGTGTTGTTCCAAGGTACTATTAAGTCCAATTTGGACCCATTCAACGAAT |
| | CCACAGAATCAGAATTGTGGGATGCTTTGAGAAGATCTGGTTTGATTACTCCCGAAGAAATG |
| | ATCAAAATGAAGGACGAGAATGAGAACGAGTACTCCAAGTTTCACTTGAATTCCGTTGTAGA |
| | AGATGAGGGCTCTAACTTTTCATTGGGTGAAAGACAATTATTGGCTTTGGCAAGAGCATTGG |
| | TCAGAAGGTCTAAGATCTTGATTATGGATGAAGCCACTTCCTCCGTTGATTACAAAACTGATT |
| | CTTTGGTCCAAGAGACTATCGCTAGAGAATTTTCTGATTGCACCATTTTGTGCGTTGCCCATA |
| | GATTGAAAACCATCATTAAGTACGACAGGATCTTGGTGTTGGAAAAAGGTGAGTTGGAAGA |
| | ATTTGACAAGCCATTGGATTTGTTCAAGAAGCAGGGTATCTTCAGAGATATGTGCAAGATTT |
| | CTAACATCGGTGTCGAGGATTTCTAA |
| SEQ ID | MSSNTSMKDEDDYQDLEKSNAHIQQPRPVKRLLTPLLTKYVPPIPQESERTRFPFYHTNIFSKALF |
| NO: 912 | AWLLPLLFKGYKRTLQQEDLWKLDEHTSIDHVYIKFEKHLNEEWLKFDAKHDPIKNPDAFPRFAIF |
| | MALMKTFKYEYTVAIVTKIISNALSAFTPLVSKKLISFISEKALVPDTPINKGIGYAFGITFMLMFSAIF |
| | MNQSLLHSKFVGGHSRTILTKALIQKSLIANAETRFHYPSGRIISFMSADLQRIDESLFELPTGFTTL |
| | EPIIIAIVLLIVNIGVSALAGIAIFFLTLILMGVPAGSLFKIREAANVFTDQRVGKMREVIQSMKMIKF |
| | YSWEDAYENLITGIRSKESSLVLKFQLTINVMITIAINASSITSMGAFLVLYAVKSHGNPANVFSSLS |
| | LFGILSQQVIELPMVFSSAAEGLLSLDRITKYLRSPVETFDVENFYDSELIKNDEIAVQIENGEFEWE |
| | LFTEIKEDDEETKKQKKKDEKQRKKELKKSQGGNGWFNKKTKTTEDSSNDEINKESSTDEDNTNQ |
| | QKPFKLSNINLKISKGEFIVVTGPIGSGKSSLLSAISAFMTKTDGKIAINGSNLLCGAPWVQNTTIRE |
| | NVLFGSKFDHVKYKKVLEVCSLEHDLKSLLAGDMTEIGERGVTLSGGQKARVNLARAVYADKEVY |
| | LFDDILSAVDANVGKNITENCLLGLLSSKTIIIATHQLSLISKADRVVFLNGDGLIDVGTESELRSKNK |
| | DFVKLMEYNKELEQKNTDDEEQINDKITKVTSIADKPSGPIDGTLFGEEERAFDSIPLSLYKQYVKA |
| | GQGMFGFTAFPLTIICIILSVFVNLFTNVWLSFWVAQKFKNLSNGQYIGLYVMFTVLSVLFVVVEL |
| | AIMGYVFTEASKTLNLKAMQKVLHSPMSFIDTTPVGRIINRFSKDTNSLDNEIGMQLKLFLHFSSTI |
| | IGIIILAIIYLPWFAIAVPFLAIFFLCATNFYQASSREVKRLEAINRSFVYNNFNEVMNGMNTIKAYG |
| | AGERFIRKNDIFGDQLNEVYFVVVSNQRWIAVNLDIMATAVVFIVAMLSVTGQFSINASSVGLLT |
| | YYMIELSQMLSFLMQTYSEVENEMNSVERVCHYANDLEQESAYRTLDYQPRPTWPEEGGIKFD |
| | NLSLRYRDGLPLVLKNLSIDIKGGEKIGICGRTGAGKSSLMIALYRIAEFAEGGIFIDGTDISKLGLHD |
| | LRSKLSIIPQDPVLFQGTIKSNLDPFNESTESELWDALRRSGLITPEEMIKMKDENENEYSKFHLNS |
| | VVEDEGSNFSLGERQLLALARALVRRSKILIMDEATSSVDYKTDSLVQETIAREFSDCTILCVAHRLK |
| | TIIKYDRILVLEKGELEEFDKPLDLFKKQGIFRDMCKISNIGVEDF |
| SEQ ID | ATGGCCAAGGATGGTATCGTTACTTCTACTGAAGCTCCATTGAAAGATGCTGAATCTGGTCA |
| NO: 913 | ATTGGTTTTGGAGAGAAGATTATTGACCCCTCTGTTGTCTAAAAAGGTTCCACCAATTCCAAC |
| | CGACGAAGAAAGAAAGTTTTACCCATTCAAGAAGGCTAACCCAATCTCCAAAGTTTTCTTTTG |
| | GTGGTTGAACCCAATCATGAACGTTGGTTACAAGAGAACTTTGACCCCACAAGATTTGTTCA |
| | AGTTGACTCCAGATATGACCATCGATCATACCTACGAAAAGTTCGATAGATACTTGACCAAG |
| | ATCGTCGAAAAAGATAGAGCTGCTGCTTTGAAAAAGGATCCATCTTTAACTCCAGAGGACTT |
| | GGAAAGAAGAGAATACCCAAAGTTCGCCATTATTAAGGCTTTGTTCTTGACCTTCAAGTGGG |
| | AATACTCTACCGCTATTATGTTCAAGGTTTTCGCTGATGTTTGTGGTGTCTGTAATCCCTTGTT |
| | GTCCAAAGAATTGATCAAGTTCGTTTCCCGTAAGACCTTGAATGCTGATATTGCTGTTAATGA |
| | TGGTGTTGGTTACGCTTTCGGTTGTACTTTGTTGTTGGCTTTCTCTGGCATCTTCATTAACCAG |
| | TTCTTGCATTTGTCTATTACTACCGGTGCTCATTGCAAGGGTATTTTGACTACTGCTTTGCTGA |
| | AAAAGTCCTTCAGAGCTGATGCTGAAACTAGACATAAGTTTACCTCTGGTAGAATCACCTCTT |
| | TGATGTCTACTGATTTGGCCAGAATTGATTTGGCTATCGGTTTACAACCATTCGGTTGGACAT |
| | TTCCAATTCCAGTTATTATTGCCATTGCCTTGTTGATCGTTAATATCGGTGTTGCTTCTTTGGC |
| | TGGTATTGCCGTTTTCATTATCTCCATTTTGGTTATTGGTGGTTCTGCTAAGGCTCTGTTGAAA |
| | ATGAGAAGAGGTGCTAACAAGTTCACCGACAAGAGAATTTCTTTGATGAGGGAAATCCTGC |
| | AGTCCATGAAGATGATTAAGTACTACTCTTGGGAAGATGCCTACGAATCTTCTGTTGTTGAAC |
| | AGAGAAATTCCGAAGTTGGTGTCATCTTGAAGATGCAGTCTATCAGAAACTTCTTGCTGGCC |
| | TTCTCTATTTCTTTGCCATCTTTCACTTCCATGATCGCTTTCTTGGTCTTGTACGGTATTTCCTCT |
| | AATAGAAACCCAGCCAACATCTTCCCTTCCATTTCTTTGTTTGGTTCCTTGGCTCAACAAACCA |
| | TGATGTTGCCAATGGCTTTGGCTACTGGTACTGATGCTATGATTGGTTTGAATAGAGTCAGG |
| | GAATTCTTGCAATCTGGTGTTGATTTGGAAGATCCTGAAGCACCACAAGGTAATGATCAAGA |
| | TTCTCAAGATGCCAACGTTGAAAAGTTGCCAGAAGATGTAGCTTTGTCTGTTAAGAACGCTA |
| | CCTTCATTTGGGAAACCTTTGATGATGAAGAGGATGAAGGTGCTGATAAGCCAAAAGCTGAT |
| | ACTGCTACTGAAAAGAAGGATTCCGATATTGCTACTCCAGCTACTTCTACCAAAGATACCCAT |
| | TCTGATTCCGAATTGAAGAACACTGCTTCTTCTACCGAAGAAGAAGGTCACGAATCTTACACT |
| | AAGTCTGTTTTCGAAGGTTTCCACAACATCAACTTGGATGTTAAGAAGGGTGAATTCGTTATT |
| | GTCACTGGTGCTATTGGTTCCGGTAAATCCTCTTTGTTGATTGCTTTAGCTGGTTTCATGAAG |
| | CAAACTGGTGGTACTTTAACTGCTGCTGAAGATGTTTTGTTGTGTGGTGCTCCATGGGTTCAA |
| | AACACTACTGTTAGAGAAAACATCACCTTCGGTTTGCCATACGAAGAGGAAAGATACGAAA |
| | GGGTTATTGATGCTTGTGCCTTGAGAGATGATTTGAAGTTGTTTGCCGGTGGTGATTTGACT |
| | GAAATTGGTGAAAGAGGTATCACTTTGTCTGGTGGTCAAAAGGCTAGAATCAATTTGGCTAG |
| | AGCTGTTTACGCTGATAAGTCCATCGTTTTGTTCGATGATGTTCTGTCTGCTGTTGATGCTAG |
| | AGTTGGTAAGCACATTATTGATGACTGTTTCGGTGAGTACATGAAGGGTAAGACTAGAGTTT |
| | TAGCTACCCACCAATTGTCCTTGGTTGATAAGGCTGATAGAGTCGTCTTTTTGAATGGTGATG |
| | GTACATTGCATATCGGTACTGTTGAAGAGTTGTTGACTTCCAATGAAGGCTTCATCAAGCTG |
| | ATGGAATTCTCCAAGAAATCCTCCGAAGATGACGAAGAAGAGGACGAGGATATTGATGAAG |
| | AAGAACAAGAAATTATCGCCCTGCAAAAGTCTCAATCCTTGGCTGTTATTCAGTCCAAAAAG |
| | AACAACAATGATGCTGCTGCTGGTGTTTTGGTTAACGAGGAAGAAAGAGCCAAAAACAAGA |
| | TCTCCTCTAAGGTTTACACCGAGTATTTGAGAGAAGGTGGTGGTATTTTGGGTAAATTTGCT |
| | GCTCCAATTGCCATCCTGTTGCTGATTTTGGATGTTTTCACTACCATTTTCATCAACGTCTGGT |
| | TGTCTTTCTGGATTACTTACAAGTGGAAGAACAGATCCGATGGTTTCTACATCGGTTTCTACG |
| | TTATGTTCGTTGTCCTGAATATCTGCTTCATTGCCTCTTGTTTCGTCTTGTTGGGTTACATTTCT |
| | ACTACCTCTGCTAGGGAATTGAACTTGAAAGCTATGAGAAGAATCTTGCATGCTCCTATGGC |
| | TTATTTGGATGTAACTCCAATGGGCAGAATTTTGAACAGATTCACTAAGGATACCGACGTCTT |
| | GGATAATGAATTGGGTGAACAATTGAGGTTGTTCTTGCACCCAACTGCTTTTGTTATCGGTGT |
| | CATTATTCTGTGCATCATCTACTTGCCATGGTTCGCTTTAGTTATTCCACCATTATTGGTCGTTT |
| | TCTCTTGCGTTACCTCTTACTACCAATCCTCATCTAGAGAAGTCAAGAGATTGGAAGCTGTTC |
| | AAAGGTCTTTCGTCTACAACAATTTCAACGAAGTGTTGAACGGTATGTCTACCTTGAAGGCTT |
| | ATAGAGCTACCTCTAGGTTCCTGAAGAAAAACAACGTTTCCGTTGACAGAATGAACGAAGCT |
| | TACTTCGTTGTTATCGCCAATCAGAGATGGATCTCCATTCATATGGATATGGTTGCTGTTTGC |
| | TTGTTGTTTGTTGTAGCTATGTTGGCCGTTACCAGACAATTTTCTATTTCTGCTGCTTCTGCAG |
| | GTTTGGTTGTTACTTACGTTATGCAAATAGGTGGCCTGATGTCCTTGATTATGAGAGCTTATA |
| | CAACCGTCGAAAACGAGATGAATTCCGTTGAAAGATTGTGCCAATACGCCAACGATTTGGTT |
| | CAAGAAAAACCATACAGGATCAACGAGACAAAACCATCTCCATCTTGGCCTGAATCAGGTTC |
| | TATTGAATTCGAAGGTGTCTCCTTGAGATATAGAGATGGCTTGCCATTGGTCTTGAGAAATTT |
| | GACTTTGGCTGTTGCTGGTGGTGAGAAGATTGGTATTTGTGGTAGAACTGGTGCTGGTAAGT |
| | CATCTATTATGACTGCCTTGTACAGGTTGTCTGAATTGGCTGAAGGTAGGATTTTGATTGATG |
| | GTTTGGACATCTCTAAGATGGGTTTGTTCGAATTGAGGTCCAAGTTGTCCATTATTCCACAAG |
| | ATCCAGTTTTGTTCCAGGGCACTATTAGAAGAAACTTGGATCCATTTGGTGAATCCGATGATC |
| | AACATTTGTGGGATTCTTTGCGTAGAGCTGGTTTGATCGATTCTTCTGTTTTGGCTACAATCA |
| | AGGCCCAAGGTAAAGAAGATAAGAACTTCCATAAGTTCCACTTGGATCAAGCTGTAGAAGA |
| | TGATGGTTCTAACTTCAGTTTGGGTGAAAGACAATTATTGGCATTGGCAAGAGCTTTGGTCA |
| | GAAACTCCAGAATATTGATCTTGGATGAAGCCACTTCCTCCGTTGATTACGAAACAGATGCTA |
| | AGATTCAGAGCACCATCAAGTCTGAATTCTCTGAATGTACGATCTTGTGCATTGCCCATAGAC |
| | TGAAAACCATTTTGGATTACGACAAGATCTTAGTCTTGGAAGCCGGTGAAATCGAAGAATTT |
| | GGTACTCCAATGACCTTGTACGAAAACGACGGTATTTTCAGACAAATGTGCGATAGATCCGA |
| | TATCACTAGGGAAGATTTTGTTCACGACCTGTGA |
| SEQ ID | MAKDGIVTSTEAPLKDAESGQLVLERRLLTPLLSKKVPPIPTDEERKFYPFKKANPISKVFFWWLNP |
| NO: 914 | IMNVGYKRTLTPQDLFKLTPDMTIDHTYEKFDRYLTKIVEKDRAAALKKDPSLTPEDLERREYPKFA |
| | IIKALFLTFKWEYSTAIMFKVFADVCGVCNPLLSKELIKFVSRKTLNADIAVNDGVGYAFGCTLLLAF |
| | SGIFINQFLHLSITTGAHCKGILTTALLKKSFRADAETRHKFTSGRITSLMSTDLARIDLAIGLQPFG |
| | WTFPIPVIIAIALLIVNIGVASLAGIAVFIISILVIGGSAKALLKMRRGANKFTDKRISLMREILQSMK |
| | MIKYYSWEDAYESSVVEQRNSEVGVILKMQSIRNFLLAFSISLPSFTSMIAFLVLYGISSNRNPANIF |
| | PSISLFGSLAQQTMMLPMALATGTDAMIGLNRVREFLQSGVDLEDPEAPQGNDQDSQDANVE |
| | KLPEDVALSVKNATFIWETFDDEEDEGADKPKADTATEKKDSDIATPATSTKDTHSDSELKNTASS |
| | TEEEGHESYTKSVFEGFHNINLDVKKGEFVIVTGAIGSGKSSLLIALAGFMKQTGGTLTAAEDVLLC |
| | GAPWVQNTTVRENITFGLPYEEERYERVIDACALRDDLKLFAGGDLTEIGERGITLSGGQKARINL |
| | ARAVYADKSIVLFDDVLSAVDARVGKHIIDDCFGEYMKGKTRVLATHQLSLVDKADRVVFLNGD |
| | GTLHIGTVEELLTSNEGFIKLMEFSKKSSEDDEEEDEDIDEEEQEIIALQKSQSLAVIQSKKNNNDAA |
| | AGVLVNEEERAKNKISSKVYTEYLREGGGILGKFAAPIAILLLILDVFTTIFINVWLSFWITYKWKNR |
| | SDGFYIGFYVMFVVLNICFIASCFVLLGYISTTSARELNLKAMRRILHAPMAYLDVTPMGRILNRFT |
| | KDTDVLDNELGEQLRLFLHPTAFVIGVIILCIIYLPWFALVIPPLLVVFSCVTSYYQSSSREVKRLEAV |
| | QRSFVYNNFNEVLNGMSTLKAYRATSRFLKKNNVSVDRMNEAYFVVIANQRWISIHMDMVAV |
| | CLLFVVAMLAVTRQFSISAASAGLVVTYVMQIGGLMSLIMRAYTTVENEMNSVERLCQYANDLV |
| | QEKPYRINETKPSPSWPESGSIEFEGVSLRYRDGLPLVLRNLTLAVAGGEKIGICGRTGAGKSSIMT |
| | ALYRLSELAEGRILIDGLDISKMGLFELRSKLSIIPQDPVLFQGTIRRNLDPFGESDDQHLWDSLRRA |
| | GLIDSSVLATIKAQGKEDKNFHKFHLDQAVEDDGSNFSLGERQLLALARALVRNSRILILDEATSSV |
| | DYETDAKIQSTIKSEFSECTILCIAHRLKTILDYDKILVLEAGEIEEFGTPMTLYENDGIFRQMCDRSD |
| | ITREDFVHDL |
| SEQ ID | ATGCCCGAGGCCAAGCTTAACAATAACGTCAACGACGTTACTAGCTACTCCTCCGCGTCTTCT |
| NO: 915 | TCTACTGAAAACGCTGCTGATCTACACAATTATAATGGGTTCGATGAGCATACAGAAGCTCG |
| | AATCCAAAAACTGGCAAGGACTCTGACCGCACAGAGTATGCAAAACTCCACTCAATCGGCAC |
| | CCAACAAAAGTGATGCTCAGTCTATATTTTCTAGCGGTGTGGAAGGTGTAAACCCGATATTC |
| | TCTGATCCTGAAGCTCCAGGCTATGACCCAAAATTGGACCCCAACTCCGAAAATTTTTCTAGT |
| | GCCGCCTGGGTTAAGAATATGGCTCACCTAAGTGCGGCAGACCCTGACTTTTATAAGCCTTA |
| | TTCCTTAGGTTGCGCTTGGAAGAACTTAAGTGCTTCTGGTGCTTCCGCAGATGTCGCCTATCA |
| | GTCAACTGTGGTTAATATTCCATACAAAATCCTAAAAAGTGGGCTGAGAAAGTTTCAACGTT |
| | CTAAAGAAACCAATACTTTCCAAATCTTGAAACCAATGGATGGTTGCCTAAACCCAGGTGAA |
| | TTGCTAGTCGTTTTAGGTAGACCAGGCTCTGGCTGTACTACTTTATTAAAATCCATCTCTTCAA |
| | ATACTCATGGTTTTGATCTTGGTGCAGATACTAAAATTTCTTACAGCGGCTACTCAGGTGATG |
| | ATATTAAGAAACATTTTCGTGGTGAAGTTGTTTACAACGCAGAAGCTGATGTACATCTGCCTC |
| | ATTTAACAGTCTTCGAAACTTTGGTTACAGTAGCGAGGTTGAAAACCCCACAGAACCGTATC |
| | AAGGGTGTCGATAGGGAAAGTTATGCGAATCATTTGGCGGAAGTAGCAATGGCAACGTACG |
| | GTTTATCGCATACAAGGAATACAAAAGTTGGTAACGACATCGTCAGAGGTGTTTCCGGTGGT |
| | GAAAGGAAGCGTGTCTCCATTGCTGAAGTCTCCATCTGTGGATCCAAATTTCAATGCTGGGA |
| | TAATGCTACAAGGGGTTTGGATTCCGCTACCGCTTTGGAATTTATTCGTGCCTTAAAGACTCA |
| | AGCTGATATTTCCAATACATCTGCCACAGTGGCCATCTATCAATGTTCTCAAGATGCGTACGA |
| | CTTGTTCAATAAAGTCTGTGTTTTGGATGATGGTTATCAGATCTACTATGGCCCCGCCGATAA |
| | GGCCAAGAAGTACTTTGAAGATATGGGGTATGTTTGTCCAAGCAGACAAACCACCGCAGATT |
| | TTTTGACCTCAGTTACAAGTCCCTCTGAGAGAACCCTGAACAAAGATATGCTAAAAAAAGGT |
| | ATTCATATACCACAGACCCCGAAGGAAATGAACGATTACTGGGTAAAATCTCCAAATTACAA |
| | AGAGCTAATGAAAGAAGTCGACCAACGATTATTGAATGACGATGAAGCAAGCCGTGAAGCT |
| | ATTAAGGAAGCCCACATTGCTAAGCAGTCCAAGAGAGCAAGACCTTCCTCTCCTTATACTGTC |
| | AGCTACATGATGCAAGTTAAATACCTATTAATCAGAAATATGTGGAGACTGCGAAATAATAT |
| | CGGGTTTACATTATTTATGATTTTGGGTAACTGTAGTATGGCTTTAATCTTGGGTTCAATGTTT |
| | TTCAAGATCATGAAAAAGGGTGATACTTCTACATTCTATTTCCGTGGTTCTGCTATGTTTTTTG |
| | CAATTCTATTCAATGCATTTTCTTCTCTGTTAGAAATCTTTTCGTTATATGAGGCCAGACCAAT |
| | CACTGAAAAACATAGAACATATTCGTTATACCATCCAAGTGCTGACGCTTTTGCATCAGTTCT |
| | ATCAGAAATACCCTCAAAGTTAATCATCGCTGTTTGCTTCAATATAATCTTCTATTTCTTAGTA |
| | GACTTTAGAAGAAATGGTGGTGTATTCTTTTTCTACTTATTAATAAACATTGTCGCGGTTTTCT |
| | CCATGTCTCACTTGTTTAGATGTGTTGGTTCCTTAACAAAGACATTGTCAGAAGCTATGGTTC |
| | CCGCTTCTATGTTATTGTTGGCTCTATCCATGTATACCGGTTTTGCTATTCCTAAGAAGAAGAT |
| | CCTACGTTGGTCTAAATGGATTTGGTATATCAATCCGTTGGCTTACTTATTCGAATCTTTGTTA |
| | ATTAACGAGTTTCATGGTATAAAATTCCCCTGCGCTGAATATGTTCCTCGTGGTCCTGCGTAT |
| | GCAAACATTTCTAGTACAGAATCTGTTTGTACCGTGGTTGGAGCTGTTCCAGGCCAAGACTA |
| | TGTTCTGGGTGATGATTTCATTAGAGGAACTTATCAATACTACCACAAAGACAAATGGCGTG |
| | GTTTCGGTATTGGTATGGCTTATGTCGTCTTCTTTTTCTTTGTCTATCTATTCTTATGTGAATAC |
| | AACGAGGGTGCTAAACAAAAAGGTGAAATATTAGTTTTCCCACGCAGTATAGTTAAAAGAAT |
| | GAAGAAAAGAGGTGTACTAACTGAAAAGAATGCAAATGACCCCGAAAACGTTGGGGAACG |
| | TAGTGACTTATCCAGCGATAGGAAAATGCTACAAGAAAGCTCTGAAGAGGAATCCGATACTT |
| | ACGGAGAAATTGGTTTATCCAAGTCAGAGGCTATATTTCACTGGAGAAACCTTTGTTACGAA |
| | GTTCAGATTAAGGCCGAAACAAGACGTATTTTGAACAATGTTGATGGTTGGGTTAAACCAGG |
| | TACTTTAACAGCTTTAATGGGTGCTTCAGGTGCTGGTAAAACCACACTTCTGGATTGTTTGGC |
| | CGAAAGGGTTACCATGGGTGTTATAACTGGTGATATCTTGGTCAATGGTATTCCCCGTGATA |
| | AATCTTTCCCAAGATCCATTGGTTATTGTCAGCAACAAGATTTGCATTTGAAAACTGCCACTG |
| | TGAGGGAGTCATTGAGATTTTCTGCTTACCTACGTCAACCAGCTGAAGTTTCCATTGAAGAA |
| | AAGAACAGATATGTTGAAGAAGTTATTAAAATTCTTGAAATGGAAAAATATGCTGATGCTGT |
| | TGTTGGTGTTGCTGGTGAAGGTTTAAACGTTGAACAAAGAAAAAGATTAACCATTGGTGTTG |
| | AATTAACTGCCAAACCAAAACTGTTGGTCTTTTTAGATGAACCTACTTCTGGTTTGGATTCTCA |
| | AACTGCTTGGTCTATTTGTCAGCTAATGAAAAAGTTGGCAAATCATGGTCAAGCAATTCTATG |
| | TACTATTCACCAACCCTCTGCTATTTTGATGCAAGAATTCGATCGTTTACTATTTATGCAACGT |
| | GGTGGTAAGACTGTCTACTTTGGCGACTTGGGCGAAGGTTGTAAAACTATGATCGATTATTT |
| | TGAAAGCCATGGTGCTCATAAATGCCCTGCTGACGCCAACCCAGCTGAATGGATGCTAGAAG |
| | TTGTTGGTGCAGCTCCAGGCTCTCATGCAAATCAAGATTATTACGAAGTTTGGAGGAATTCT |
| | GAAGAGTACAGGGCCGTTCAATCTGAATTAGATTGGATGGAAAGAGAATTACCAAAGAAAG |
| | GTTCGATAACTGCAGCTGAGGACAAACACGAATTTTCACAATCAATTATTTATCAAACAAAAT |
| | TGGTCAGTATTCGTCTATTCCAGCAATATTGGAGATCTCCAGATTATTTATGGTCGAAGTTTA |
| | TTTTAACTATTTTCAATCAATTGTTCATCGGTTTCACTTTCTTCAAAGCAGGAACCTCGCTACA |
| | GGGTTTACAAAATCAAATGTTGGCTGTGTTCATGTTTACGGTTATTTTCAATCCTATTCTACAA |
| | CAATACCTACCATCTTTTGTCCAGCAAAGAGATTTGTATGAGGCCAGGGAACGCCCCTCAAG |
| | GACTTTTTCTTGGATTTCATTTATCTTCGCTCAAATATTCGTGGAAGTTCCATGGAATATATTG |
| | GCAGGTACTATTGCTTATTTTATCTACTATTATCCAATTGGATTTTACTCCAACGCGTCTGCAG |
| | CTGGCCAGTTGCATGAAAGGGGTGCTTTATTTTGGTTGTTCTCTTGTGCTTTCTACGTTTATGT |
| | TGGTTCTATGGGTCTGCTTGTCATTTCATTCAACCAAGTTGCAGAAAGTGCAGCTAACTTAGC |
| | CTCTTTGTTGTTTACAATGTCTTTGTCTTTTTGTGGTGTTATGACTACCCCAAGTGCCATGCCT |
| | AGATTTTGGATATTCATGTACAGGGTTTCACCTTTGACTTATTTCATTCAGGCTCTGTTGGCTG |
| | TTGGTGTTGCTAACGTAGACGTCAAATGCGCTGATTACGAATTGCTAGAATTCACACCACCAT |
| | CCGGTATGACATGTGGGCAGTACATGGAACCATATTTACAACTAGCAAAGACTGGTTACTTA |
| | ACTGATGAAAATGCCACTGACACCTGTAGTTTCTGTCAAATATCTACAACCAATGATTACTTA |
| | GCTAATGTCAATTCTTTCTACAGTGAGAGATGGAGAAATTATGGTATCTTCATCTGTTATATT |
| | GCATTCAATTATATCGCTGGTGTCTTTTTCTACTGGTTAGCAAGAGTGCCTAAAAAGAACGGT |
| | AAACTCTCCAAGAAATAA |
| SEQ ID | MPEAKLNNNVNDVTSYSSASSSTENAADLHNYNGFDEHTEARIQKLARTLTAQSMQNSTQSAP |
| NO: 916 | NKSDAQSIFSSGVEGVNPIFSDPEAPGYDPKLDPNSENFSSAAWVKNMAHLSAADPDFYKPYSL |
| | GCAWKNLSASGASADVAYQSTVVNIPYKILKSGLRKFQRSKETNTFQILKPMDGCLNPGELLVVL |
| | GRPGSGCTTLLKSISSNTHGFDLGADTKISYSGYSGDDIKKHFRGEVVYNAEADVHLPHLTVFETL |
| | VTVARLKTPQNRIKGVDRESYANHLAEVAMATYGLSHTRNTKVGNDIVRGVSGGERKRVSIAEV |
| | SICGSKFQCWDNATRGLDSATALEFIRALKTQADISNTSATVAIYQCSQDAYDLFNKVCVLDDGY |
| | QIYYGPADKAKKYFEDMGYVCPSRQTTADFLTSVTSPSERTLNKDMLKKGIHIPQTPKEMNDYW |
| | VKSPNYKELMKEVDQRLLNDDEASREAIKEAHIAKQSKRARPSSPYTVSYMMQVKYLLIRNMWR |
| | LRNNIGFTLFMILGNCSMALILGSMFFKIMKKGDTSTFYFRGSAMFFAILFNAFSSLLEIFSLYEARP |
| | ITEKHRTYSLYHPSADAFASVLSEIPSKLIIAVCFNIIFYFLVDFRRNGGVFFFYLLINIVAVFSMSHLFR |
| | CVGSLTKTLSEAMVPASMLLLALSMYTGFAIPKKKILRWSKWIWYINPLAYLFESLLINEFHGIKFP |
| | CAEYVPRGPAYANISSTESVCTVVGAVPGQDYVLGDDFIRGTYQYYHKDKWRGFGIGMAYVVFF |
| | FFVYLFLCEYNEGAKQKGEILVFPRSIVKRMKKRGVLTEKNANDPENVGERSDLSSDRKMLQESS |
| | EEESDTYGEIGLSKSEAIFHWRNLCYEVQIKAETRRILNNVDGWVKPGTLTALMGASGAGKTTLL |
| | DCLAERVTMGVITGDILVNGIPRDKSFPRSIGYCQQQDLHLKTATVRESLRFSAYLRQPAEVSIEEK |
| | NRYVEEVIKILEMEKYADAVVGVAGEGLNVEQRKRLTIGVELTAKPKLLVFLDEPTSGLDSQTAWS |
| | ICQLMKKLANHGQAILCTIHQPSAILMQEFDRLLFMQRGGKTVYFGDLGEGCKTMIDYFESHGA |
| | HKCPADANPAEWMLEVVGAAPGSHANQDYYEVWRNSEEYRAVQSELDWMERELPKKGSITA |
| | AEDKHEFSQSIIYQTKLVSIRLFQQYWRSPDYLWSKFILTIFNQLFIGFTFFKAGTSLQGLQNQMLA |
| | VFMFTVIFNPILQQYLPSFVQQRDLYEARERPSRTFSWISFIFAQIFVEVPWNILAGTIAYFIYYYPIG |
| | FYSNASAAGQLHERGALFWLFSCAFYVYVGSMGLLVISFNQVAESAANLASLLFTMSLSFCGVM |
| | TTPSAMPRFWIFMYRVSPLTYFIQALLAVGVANVDVKCADYELLEFTPPSGMTCGQYMEPYLQL |
| | AKTGYLTDENATDTCSFCQISTTNDYLANVNSFYSERWRNYGIFICYIAFNYIAGVFFYWLARVPK |
| | KNGKLSKK |
| SEQ ID | ATGGCCTTGAGATCTTTCTGTTCTGCTGATGGTTCTGATCCATTGTGGGATTGGAATGTTACT |
| NO: 917 | TGGCATACTTCTAATCCAGATTTCACCAAGTGTTTCCAAAACACCGTTTTGACTTGGGTTCCAT |
| | GTTTTTACTTGTGGTCTTGCTTTCCCTTGTACTTCTTCTACTTGTCCAGACATGATAGAGGTTA |
| | CATCCAAATGACCCATTTGAACAAGACTAAGACTGCTTTGGGTTTCTTCTTGTGGATTATTTG |
| | TTGGGCCGATCTGTTTTACTCTTTCTGGGAAAGATCACAGGGTGTTTTGAGAGCACCAGTTTT |
| | GTTGGTTTCTCCAACTTTGTTGGGTATCACTATGTTGTTGGCTACCTTCTTGATTCAGTTGGAG |
| | AGAAGAAAAGGTGTCCAATCCTCTGGTATTATGTTGACTTTTTGGTTGGTTGCTTTGTTGTGC |
| | GCTTTGGCTATTTTGAGATCCAAGATTATTTCCGCCTTGAAAAAGGATGCCCACGTTGATGTT |
| | TTTAGAGACTCTACCTTCTACCTGTACTTCACCTTGGTTTTGGTTCAATTGGTTCTGTCTTGTTT |
| | CTCTGATTGCTCTCCTTTGTTCTCTGAAACCGTTCATGATAGAAATCCCTGTCCAGAATCTTCT |
| | GCTTCATTCTTGTCTAGAATTACCTTCTGGTGGATCACTGGTATGATGGTTCATGGTTATAGA |
| | CAACCCTTGGAATCTTCAGATTTGTGGTCCTTGAACAAAGAAGATACCTCCGAAGAAGTTGTT |
| | CCAGTTTTGGTTAACAACTGGAAGAAAGAATGCGACAAGTCCAGAAAACAACCAGTTAGAA |
| | TAGTTTACGCTCCACCAAAAGATCCATCTAAGCCAAAAGGTTCTTCCCAATTGGATGTTAACG |
| | AAGAAGTCGAAGCTTTGATCGTTAAGTCTCCACATAAGGATAGAGAGCCCTCTTTGTTTAAG |
| | GTGTTGTACAAAACTTTCGGCCCCTACTTTTTGATGTCCTTCTTGTATAAGGCCTTGCACGATT |
| | TGATGATGTTTGCTGGTCCAAAGATCTTGGAGCTGATTATTAACTTCGTCAACGATAGAGAA |
| | GCTCCAGATTGGCAAGGTTATTTCTATACTGCCTTGTTGTTTGTTTCCGCTTGCTTGCAAACTT |
| | TGGCATTGCATCAATACTTCCACATCTGTTTCGTTTCCGGTATGAGAATCAAAACTGCTGTTG |
| | TTGGTGCTGTTTACAGAAAGGCTTTGTTGATTACTAATGCCGCCAGAAAATCTTCTACTGTCG |
| | GTGAAATAGTCAACTTGATGTCTGTTGATGCCCAAAGATTCATGGATTTGGCCACTTACATTA |
| | ACATGATTTGGTCTGCTCCATTGCAAGTTATTCTGGCCTTGTATTTCTTGTGGTTGTCTTTGGG |
| | TCCATCAGTTTTGGCTGGTGTTGCTGTTATGATTTTGATGGTTCCATTGAACGCTGTTATGGC |
| | TATGAAAACTAAGACCTACCAAGTTGCCCATATGAAGTCTAAGGATAACAGGATCAAACTGA |
| | TGAACGAGATCTTGAACGGTATCAAGGTTTTGAAGTTGTACGCTTGGGAATTAGCCTTCCAA |
| | GATAAGGTCATGTCTATCAGGCAAGAAGAATTGAAGGTCTTGAAGAAGTCTGCTTATTTGGC |
| | TGCAGTTGGTACTTTTACTTGGGTGTGTACTCCATTCTTGGTTGCCTTGTCTACTTTTGCTGTT |
| | TTCGTTACTGTTGACGAGAGAAACATTTTGGATGCTAAGAAGGCTTTCGTTTCTTTGGCCTTG |
| | TTCAACATCTTGAGATTCCCATTGAACATCTTGCCAATGGTCATCTCTTCTATCGTTCAAGCTT |
| | CTGTCTCTTTGAAGAGATTGAGGATCTTCTTGTCCCACGAAGAATTGGAACCAGATTCTATCG |
| | AAAGAAGGTCCATTAAGTCTGGTGAAGGTAACTCTATTACCGTTAAGAACGCTACTTTCACAT |
| | GGGCTAGAGGTGAACCACCAACTTTGAATGGTATTACTTTCTCCATTCCAGAAGGTGCTTTA |
| | GTTGCAGTTGTTGGTCAAGTTGGTTGTGGTAAATCATCTTTGTTGTCTGCTTTATTGGCCGAA |
| | ATGGATAAGGTTGAAGGTCACGTTACTTTGAAAGGTTCTGTTGCTTATGTTCCACAACAAGC |
| | CTGGATTCAAAACGATTCTTTGAGGGAAAACATCTTGTTCGGTCACCCATTACAAGAGAATTA |
| | CTACAAAGCAGTTATGGAAGCTTGCGCTTTGTTGCCAGATTTGGAAATTTTGCCATCTGGTGA |
| | TAGAACCGAAATTGGTGAAAAGGGTGTTAATTTGTCTGGTGGTCAAAAGCAGAGAGTTTCTT |
| | TAGCTAGAGCTGTCTACTCTAACTCCGATATCTATTTGTTTGACGACCCATTGTCTGCAGTTGA |
| | TGCACATGTTGGTAAGCACATCTTTGAAAAAGTTGTAGGTCCAATGGGCCTGTTGAAGAACA |
| | AAACTAGAATTTTGGTTACCCACGGCATCTCTTATTTGCCACAAGTTGATGTTATCATCGTCAT |
| | GTCCGGTGGTAAGATTTCTGAAATGGGTTCCTACCAAGAGTTGTTGGATAGAGATGGTGCTT |
| | TTGCTGAATTCTTGAGAACTTACGCTAACGCTGAACAAGATTTGGCTTCTGAAGATGATTCTG |
| | TTTCCGGTTCTGGTAAAGAATCCAAGCCAGTTGAAAATGGTATGTTGGTTACTGATACAGTC |
| | GGTAAGCACTTGCAAAGACATTTGTCTAACTCCTCTTCTCATTCCGGTGATACTTCTCAACAAC |
| | ATTCCTCTATTGCCGAATTGCAAAAAGCTGGTGCTAAAGAAGAGACTTGGAAATTGATGGAA |
| | GCTGATAAGGCTCAAACAGGTCAAGTTCAATTGTCTGTTTACTGGAACTACATGAAGGCCAT |
| | TGGTTTGTTCATCACGTTCTTGTCCATTTTCTTGTTCTTGTGCAACCATGTTTCTGCTTTGGCTT |
| | CAAATTACTGGTTGTCATTGTGGACTGATGATCCACCTGTTGTTAATGGTACTCAAGCCAATA |
| | GAAACTTCAGGTTGTCAGTTTATGGTGCCTTGGGTATTTTACAAGGTGCTGCTATTTTCGGTT |
| | ACTCCATGGCTGTTTCTATCGGTGGTATTTTTGCTTCCAGAAGATTGCACTTGGACTTGTTGTA |
| | TAACGTGTTGAGATCTCCAATGAGCTTCTTTGAAAGAACTCCATCTGGTAACTTGGTCAACAG |
| | GTTCTCCAAAGAATTAGATACCGTTGACTCCATGATTCCACAAGTCATCAAAATGTTCATGGG |
| | CAGCTTGTTTTCAGTTATTGGTGCCGTTATTATCATCTTGTTGGCAACTCCAATTGCCGCTGTT |
| | ATTATTCCACCATTGGGTCTAGTTTACTTTTTCGTCCAGAGATTCTACGTTGCCTCATCTAGGC |
| | AATTGAAAAGATTGGAATCCGTGTCTAGATCCCCAGTTTACTCTCATTTTAACGAAACCTTGT |
| | TGGGTGTCTCCGTTATTAGAGCTTTTGAGGAACAAGAGAGATTCATCCACCAATCCGATTTG |
| | AAGGTTGACGAAAATCAAAAGGCTTACTACCCATCTATTGTCGCTAACAGATGGTTGGCTGT |
| | TAGATTAGAATGTGTTGGTAACTGCATCGTTTTGTTCGCTGCTTTGTTTGCTGTCATCTCTAGA |
| | CATTCTTTGTCTGCTGGTTTAGTTGGTTTGTCCGTTTCTTACTCCTTGCAAATTACCGCTTACTT |
| | GAACTGGTTGGTCAGAATGTCATCAGAAATGGAAACTAACATCGTTGCCGTCGAAAGGTTG |
| | AAAGAATACTCCGAAACTGAAAAAGAAGCCCCTTGGCAAATTCAAGAAACTGCTCCACCATC |
| | TACTTGGCCACATTCTGGTAGAGTTGAATTCAGAGATTACTGCTTGAGATACAGGGAAGATC |
| | TGGATTTGGTTCTAAAGCACATTAACGTTACCATCGAAGGTGGTGAAAAAGTCGGTATAGTT |
| | GGTAGAACAGGTGCTGGTAAATCTTCATTGACATTGGGTTTGTTTAGGATCAACGAATCTGC |
| | TGAAGGTGAAATCATTATCGACGGTGTTAACATTGCTAAGATCGGCTTGCATAATCTGAGAT |
| | TCAAGATCACTATCATCCCACAAGATCCCGTTTTATTCTCTGGTTCTTTGAGAATGAACTTGGA |
| | CCCATTCTCTCAATACTCTGATGAAGAGGTTTGGATGGCTTTGGAATTGGCTCATTTGAAGG |
| | GTTTTGTTTCAGCTTTGCCAGATAAGTTGAACCATGAATGTGCAGAAGGCGGAGAAAATTTG |
| | TCAGTAGGTCAAAGACAATTGGTTTGCTTGGCTAGAGCTTTGTTAAGAAAGACCAAAATCCT |
| | GGTCTTGGATGAAGCTACTGCTGCTGTTGACTTAGAAACCGATAACTTGATCCAATCCACCAT |
| | CAGAACTCAATTTGAGGATTGCACTGTTTTGACCATTGCTCATAGATTGAACACCATCATGGA |
| | TTACACCAGAGTTATCGTTTTGGACAAGGGTGAAGTTAGAGAATGTGGTGCTCCATCTGAAC |
| | TATTGCAACAGAGAGGTATTTTCTACTCTATGGCTAAAGATGCCGGTTTGGTTTGA |
| SEQ ID | MALRSFCSADGSDPLWDWNVTWHTSNPDFTKCFQNTVLTWVPCFYLWSCFPLYFFYLSRHDR |
| NO: 918 | GYIQMTHLNKTKTALGFFLWIICWADLFYSFWERSQGVLRAPVLLVSPTLLGITMLLATFLIQLERR |
| | KGVQSSGIMLTFWLVALLCALAILRSKIISALKKDAHVDVFRDSTFYLYFTLVLVQLVLSCFSDCSPL |
| | FSETVHDRNPCPESSASFLSRITFWWITGMMVHGYRQPLESSDLWSLNKEDTSEEVVPVLVNN |
| | WKKECDKSRKQPVRIVYAPPKDPSKPKGSSQLDVNEEVEALIVKSPHKDREPSLFKVLYKTFGPYF |
| | LMSFLYKALHDLMMFAGPKILELIINFVNDREAPDWQGYFYTALLFVSACLQTLALHQYFHICFVS |
| | GMRIKTAVVGAVYRKALLITNAARKSSTVGEIVNLMSVDAQRFMDLATYINMIWSAPLQVILALY |
| | FLWLSLGPSVLAGVAVMILMVPLNAVMAMKTKTYQVAHMKSKDNRIKLMNEILNGIKVLKLYA |
| | WELAFQDKVMSIRQEELKVLKKSAYLAAVGTFTWVCTPFLVALSTFAVFVTVDERNILDAKKAFV |
| | SLALFNILRFPLNILPMVISSIVQASVSLKRLRIFLSHEELEPDSIERRSIKSGEGNSITVKNATFTWAR |
| | GEPPTLNGITFSIPEGALVAVVGQVGCGKSSLLSALLAEMDKVEGHVTLKGSVAYVPQQAWIQN |
| | DSLRENILFGHPLQENYYKAVMEACALLPDLEILPSGDRTEIGEKGVNLSGGQKQRVSLARAVYSN |
| | SDIYLFDDPLSAVDAHVGKHIFEKVVGPMGLLKNKTRILVTHGISYLPQVDVIIVMSGGKISEMGS |
| | YQELLDRDGAFAEFLRTYANAEQDLASEDDSVSGSGKESKPVENGMLVTDTVGKHLQRHLSNSS |
| | SHSGDTSQQHSSIAELQKAGAKEETWKLMEADKAQTGQVQLSVYWNYMKAIGLFITFLSIFLFLC |
| | NHVSALASNYWLSLWTDDPPVVNGTQANRNFRLSVYGALGILQGAAIFGYSMAVSIGGIFASRR |
| | LHLDLLYNVLRSPMSFFERTPSGNLVNRFSKELDTVDSMIPQVIKMFMGSLESVIGAVIIILLATPIA |
| | AVIIPPLGLVYFFVQRFYVASSRQLKRLESVSRSPVYSHFNETLLGVSVIRAFEEQERFIHQSDLKVD |
| | ENQKAYYPSIVANRWLAVRLECVGNCIVLFAALFAVISRHSLSAGLVGLSVSYSLQITAYLNWLVR |
| | MSSEMETNIVAVERLKEYSETEKEAPWQIQETAPPSTWPHSGRVEFRDYCLRYREDLDLVLKHIN |
| | VTIEGGEKVGIVGRTGAGKSSLTLGLFRINESAEGEIIIDGVNIAKIGLHNLRFKITIIPQDPVLFSGSL |
| | RMNLDPFSQYSDEEVWMALELAHLKGFVSALPDKLNHECAEGGENLSVGQRQLVCLARALLRK |
| | TKILVLDEATAAVDLETDNLIQSTIRTQFEDCTVLTIAHRLNTIMDYTRVIVLDKGEVRECGAPSELL |
| | QQRGIFYSMAKDAGLV |
| SEQ ID | ATGAACGTTGAAGCCGCCAACGAAAAAGAATTGCTATTGCAAAAGAGGTCCCTGACTTTCTT |
| NO: 919 | GTACGGTAAAAATGTTCCACCATTGCCATTGGAAGAAGAGAGAAAAGTTTTTCCACATAGGC |
| | ACACCAACATTATCTCTAGAGCTTTGTTCTACTACTTGAACCCAATGTTGAGAGTCGGTTACA |
| | AAAGAACATTGCAACCACAAGATATGTACGTCTTGGATGAACGTGATTCTGTTGATGCTATG |
| | TACGACAAGTTCAGAAACTACTTGGATGTCGAATTGGATAAGGCTAGAGCCAAACATATCCA |
| | AAAGAAAAGAGAAGCTAGAGCCGAATTGGGTAACACTTCTACTGTTGATGAAGATACCGAC |
| | TTGGAAGATTTCGAATTGCCTTACATCGTTATCGTCAAGGGTTTGTTTCATTTGTTTGGTTGG |
| | CAGTATATGTGGGGCTCTTTGTTGAAAGTTTTCACCGATTTGTTTTACACCCTGATGCCATTG |
| | GTTCAAAAGAGATTGGTTAACTTCGTTGAAGAATCCGCTTACGGTTTCCATCCAACTTTAGGT |
| | AAAGGTGTTGGTTACTCTATTGGTGTCGGTTTGATGGTTTATTTCGCTGGTTTGTGTGTTAAC |
| | CACTTCGTCTACAACTCTATTACTGTTGGTGCTAAGTGTAAGGCTGTTTTGACTAAGTTGTTGT |
| | TGGAGAAGTCCTTCAGATTGGATGCTAGAGGTAAACATAAGTTCCCAGTTGGTAAGATCAAC |
| | TCCATTATGGGTACTGATTTGACCAGAGTTGATTTGGCTATTGGTTTCTTCCCAATCATCTTCG |
| | AATTCCCATTCTCCATTATCCTGTGCATCATCCTGTTGTTGGTTAACATTGGTGTTTCTGCTTTG |
| | GCCGGTATTTCCTTGTTCGTTGTTATTTTGTTGTTCACCTCCTACGTTGTCAGGGTCTTGTACA |
| | AAATGAGAGTTAGAGCTAACGTTTACACCGACCAAAGAGTTAATTTGGTCAAAGAGCTGCTG |
| | AAGAACTTCAAGATGGTTAAGATGTACGGTTGGGAGAACTCTTACTTCAAGCAATTTGTTGA |
| | CACCAGGCAGAAAGAAATGACCATCGTTTTGAGAATGCAGCACATCAGAAACTTTTTGGACG |
| | CTTTGTCTTTCTGGTTGCCAATTATTACCTCCATGGTTTCTTTCTTGGTGTTGTACCATTTGAGG |
| | AACAACAGAACAGTTGGTGACATCTTCTCTTCATTGACCTTGTTCCAAGAATTGACTGGTCAA |
| | TTTGCTATGGTTACCCCATCTTTGTCTATGGCTACTGATATGGTTGTCGGTTTTAAGAGAGTT |
| | GCCCAATTGATTTCTTGTCCAGATGCTCCAGCTTTGGAAGAATTTCATGATTTGTTGGACGAC |
| | GAAAAGTTGGCTTTGAAATTGGCTCATGCTTCTTTCAAGTGGCATACCTTCGAAGAATCTGCT |
| | ACTACTGAAGTTGTTATCGTCCCAGAATCTAAGACCTCTTCATCTAAAGATGCTTCCAGAACT |
| | GAATTTCCAGGCTTGCATGATTTGACGTTGTCTATTTCTAGAGGTGAATTCATCGTTGTTACC |
| | GGTGCAATTGGTTCTGGTAAATCTTCTTTGTTGTCCGCCATTTCTGGTTTCATGCCAAAAACTG |
| | GTGGTTCTGTTGCTAAGAACGGTAGTTTGTTGTTGTGTGGTTATCCATGGGTCCAAAATGCTA |
| | CTGTTAGAGAGAATATCTTGTTCGGTCAACCATTCGACCAAACTAAGTATGACGAAATCGTTA |
| | GAGTTTGCTCCTTGGATACTGATTTCGAGTTGTTTTCTGCTGGTGATATGACCGAAGTTGGTG |
| | AAAGAGGTATTACTTTGTCTGGTGGTCAAAAGGCTAGAATCAATTTGGCTAGAGCTGTTTAC |
| | TCCGATAGAGACATCATTTTGTTGGATGATGTTTTGTCTGCTGTTGACGCTAAGGTTGGTAAA |
| | CACATTATGGAACAATGCATCTTGGGTTACTTGAAGGGTAAGACTAGAATTTTGGCTACCCA |
| | CCAATTGTCCTTGATTAACGCTGCTGATAAGGTCATTTTCTTGAATGGTAACGGTTCCATCGA |
| | TTACGGTACATTGCATGAAGTTAGATCCAGAAACTCCGCCTTCATTAGATTGATGGAATTCTC |
| | TCATGACCCCGAAGAGGAACAAAGACCAGATGAAGCTGACGAAAAGAAAGAGGATGAATT |
| | GAAGGCTGAAAAAGAAGAGGACGGTAAATTGATGAGAGATGAAGAAAGAGCCGTCAACTC |
| | TATCTCTAAGAATGTTTACTCTACCTACATCTTGTCCGGTTCAGGTAAATTGGGTTATTTGTTC |
| | CCAATCCTGATTTTGTTCGCTTGTGCTGTTTCTACCTTCTCTGATTTGTTCACTAACAACTGGTT |
| | GTCCTTCTGGCAAGATAAGAAGTTTAGAAAGCCACCAGGTTTCTACCAAGGCATCTATATTTT |
| | GTTAGGCTTCTCCACCTTGCTGTTGTTGACTTTTTACTTCGCTTTGATCGTCCAGTTCTGTAAC |
| | AAAGCTGCTAATCAATTCAACACCTCCGCTTTCCAAAATTTGTTGCATGCTCCAATGTCCTTCA |
| | TTGATACAACTCCAATGGGTAGAGTCTTGAACAGATTCACTAAGGATTCCGATGTTTTGGAC |
| | AACGAGATCCAATCTCAATTCAGGATGTTCATTCAAGAGTTTTCCGTTGTTGTCGGCACCTTG |
| | ATTTTGTGCATTATCTATTTGCCATGGTTCGCTATTGCCATTCCAGTTATGATAGTTGCCTATT |
| | ACTTGATCGCCAACTTCTACTTGGCTTCCTCTAGAGAAATCAAAAGATTGGAAGCCGTCAAG |
| | AGATCCGAAGTTTATTCTCATTTTAACGAGGCCTTGTCTGGTTTGGATACAATCAAAGCTCAT |
| | TCCAGCTCTGAAAGGTTCATGGAAACTAACTCCAGATTGATCGACCAAATGAACGAATCCTA |
| | CTTCACTTTCGTTGCTGTTCAAAGATGGTTGGCTTGCAACTTGGATATCATGGTTTCCTTCATG |
| | TGCTTGTTCATCTGCCTGTTGTGTTCCTTCAGAATCTTCCATATTAGAGGTGCTTATGCTGGTT |
| | TGCTATTGACCTGTGTTTTTAACATCGTCGGCATGTTGTCTTACATGTTAAGAGCCATGACCG |
| | AAATCGAGAATCAAATGAACTCTGTCGAAAGGTTGAAGTTTTACGCTGTCGACTTAGAACAA |
| | GAAGCCCCATTTGATATCCCTGAAAGAAATCCATCTCCATTGTGGCCTCAAAGAGGTGCTATT |
| | TGTTTTTCTAACGTCACCATGTCCTATAGAGAAGGTTTGCCACCAGCTGTTAGAAACTTGTCT |
| | TTGGATGTTGCCGGTGGTGAAAAGATAGGTGTTTGTGGTAGAACTGGTGCAGGTAAATCAT |
| | CCGTTATGTACAGCTTGTTTAGATTGGCTGAATTCGATGGTAGAATCACCATTGATGATGTG |
| | GACATTTCTAAAATCGGCTTGCACAAGTTGAGAACCTCATTGTCTATTATCCCACAAGATCCA |
| | GTTTTGTTCTCCGGTAATATCAGGTCTAATTTGGACCCTTTCCAAGAACACTCTGATGATAATT |
| | TGTGGGATGCTTTGTCTAAAGCCGGTTTGGTTGAAACTGATGCTTTGGATTTGGTTAAGCAC |
| | CAGACTAAGTCTGATAGGAACTTGCATAAGTTTCACTTGGCCAGATTGGTTGAAGATGATGG |
| | TTCTAATTTCTCCTTGGGTGAACGTCAATTATTGGCTTTGGCAAGAGCTTTGGTTAGAGGTTC |
| | TAAGATCTTGGTTTTGGATGAAGCTACTTCCTCCGTTGATTACGAAACAGATGCTAAAGTCCA |
| | AAAGACCATCACTAATGAATTCGCTGATTGCACCGTTTTGTGTATTGCCCATAGATTGAAAAC |
| | CATCGTCAAGTACGATAGGATTATGGTCTTGGACAAAGGTGAAATCGTCGAATTAGGTAAGC |
| | CAATCGAACTGTATCAACACGACGGTATTTTTAGGTCTATGTGCGAAAAGTCCGGTATTACTA |
| | GAGCTGACTTCGATATCTGA |
| SEQ ID | MNVEAANEKELLLQKRSLTFLYGKNVPPLPLEEERKVFPHRHTNIISRALFYYLNPMLRVGYKRTL |
| NO: 920 | QPQDMYVLDERDSVDAMYDKFRNYLDVELDKARAKHIQKKREARAELGNTSTVDEDTDLEDFE |
| | LPYIVIVKGLFHLFGWQYMWGSLLKVFTDLFYTLMPLVQKRLVNFVEESAYGFHPTLGKGVGYSI |
| | GVGLMVYFAGLCVNHFVYNSITVGAKCKAVLTKLLLEKSFRLDARGKHKFPVGKINSIMGTDLTR |
| | VDLAIGFFPIIFEFPFSIILCIILLLVNIGVSALAGISLFVVILLFTSYVVRVLYKMRVRANVYTDQRVNL |
| | VKELLKNFKMVKMYGWENSYFKQFVDTRQKEMTIVLRMQHIRNFLDALSFWLPIITSMVSFLVL |
| | YHLRNNRTVGDIFSSLTLFQELTGQFAMVTPSLSMATDMVVGFKRVAQLISCPDAPALEEFHDLL |
| | DDEKLALKLAHASFKWHTFEESATTEVVIVPESKTSSSKDASRTEFPGLHDLTLSISRGEFIVVTGAI |
| | GSGKSSLLSAISGFMPKTGGSVAKNGSLLLCGYPWVQNATVRENILFGQPFDQTKYDEIVRVCSL |
| | DTDFELFSAGDMTEVGERGITLSGGQKARINLARAVYSDRDIILLDDVLSAVDAKVGKHIMEQCIL |
| | GYLKGKTRILATHQLSLINAADKVIFLNGNGSIDYGTLHEVRSRNSAFIRLMEFSHDPEEEQRPDEA |
| | DEKKEDELKAEKEEDGKLMRDEERAVNSISKNVYSTYILSGSGKLGYLFPILILFACAVSTFSDLFTN |
| | NWLSFWQDKKFRKPPGFYQGIYILLGFSTLLLLTFYFALIVQFCNKAANQFNTSAFQNLLHAPMSF |
| | IDTTPMGRVLNRFTKDSDVLDNEIQSQFRMFIQEFSVVVGTLILCIIYLPWFAIAIPVMIVAYYLIAN |
| | FYLASSREIKRLEAVKRSEVYSHFNEALSGLDTIKAHSSSERFMETNSRLIDQMNESYFTFVAVQR |
| | WLACNLDIMVSFMCLFICLLCSFRIFHIRGAYAGLLLTCVFNIVGMLSYMLRAMTEIENQMNSVE |
| | RLKFYAVDLEQEAPFDIPERNPSPLWPQRGAICFSNVTMSYREGLPPAVRNLSLDVAGGEKIGVC |
| | GRTGAGKSSVMYSLFRLAEFDGRITIDDVDISKIGLHKLRTSLSIIPQDPVLFSGNIRSNLDPFQEHS |
| | DDNLWDALSKAGLVETDALDLVKHQTKSDRNLHKFHLARLVEDDGSNFSLGERQLLALARALVR |
| | GSKILVLDEATSSVDYETDAKVQKTITNEFADCTVLCIAHRLKTIVKYDRIMVLDKGEIVELGKPIELY |
| | QHDGIFRSMCEKSGITRADFDI |
| SEQ ID | ATGGACACCTCCTCCAAAGAAAACATCAGGTTGTTTTCCAAGAACTCCATGCAACCAGTTGGT |
| NO: 921 | AGATTGTCTTTCAAAACTGAGTACCCATCCTCTGAAGAAAAGCAACCATGTTGTTGTCAGCTG |
| | AAAGTTTTCTTGGGTGCTTTGTCTTTTGTTTACTTCGCTAAAGCTTTGGCCGAAGGTTACTTGA |
| | AGTCTACTATTACCCAAATCGAACGTAGATTCGACATCCCATCTTCATTGGTTGGTATTATCG |
| | ATGGTTCCTTCGAAATCGGTAACTTGTTGGTTATTGCCTTCGTTTCTTACTTCGGTGCTAAATT |
| | GCATAGGCCAAAGATTATTGGTGCTGGTTGTTTGATTATGGGTGTCGGTACTTTGTTGATTGC |
| | TACTCCACATTTCTTCATGGAACAGTACAAGTACGAAAGATACTCTCCAGCTTCTAACTCCAC |
| | CTTGTCTATTTCTCCATGTTTGTTGGACAGCAACAACTCTTTGCCAATTCCAGCTGTTGAAAAG |
| | TCCCAATCCAAGATTAACAACGAATGCCAAGTTGATGCCTCTTCTTCTATGTGGGTTTATGTC |
| | TTTTTGGGCAACTTGTTGAGAGGTTTTGGTGAAACTCCAATTCAACCATTGGGTATTGCTTAC |
| | TTGGATGATTTCGCTTCTCAAGATAATGCCGCTTTCTACATTGGTTGTGTTCAAACCGTTGCTA |
| | TTATCGGTCCAATTTTCGGTTTCTTGTTGGGTTCTTTGTGTGCTAAGTTGTACGTTGATACTGG |
| | TTTCGTTAACTTGGATCACATTACTATCACTCCAAAGGATCCACAATGGGTTGGTGCTTGGTG |
| | GTTGGGTTATTTGATTGCTGGTTCTATTACCTTGTTGGCTGCTGTTCCATTTTGGTGTTTGCCT |
| | AGAACTTTGCCAAGACCAAGATCTAGAGAAGATTCCTCTTCCTCCTCGGAAAAATCCAAGTTC |
| | ATTATGGATGACTTGACCGACTATCAAACTCCACCAGGTGAAAAAGCTAAGATTATGGAAAT |
| | GGCCAGAGACTTCTTGCCATCTTTGAAATCTTTGTTTGGTAACCCCGTCTACTTCTTGTACTTG |
| | TGTGCTTCTACTGTGCAGTTCAATTCTTTGTTCGGTATGGTTACTTACAAGCCCAAGTACATCG |
| | AACAACAATATGGTCAATCTGCTTCCAAGGCCAATTTCGTTATTGGTTTGATTAACATTCCAG |
| | CCGTTGCCTTGGGTATTTTCTCTGGTGGTATCATTATGAAGAAGTTCAGAGTCTCTATTAGAG |
| | GTGCTGCAAAGTTGTATTTGGGTTCATCTGTTTTGGGCTATCTGCTGTTTTTGTCCTTGTTTGC |
| | TTTGGGTTGTGAAAACTCTGATGTTGCTGGTTTGACCATTTCTTACCAAGGTACAAAACCAGT |
| | CTCTTACCACGAAAGAGCTTTGTTTTCTGATTGCAACTCTAGATGCAAGTGCTCTGAAACAAA |
| | ATGGGAACCTATTTGTGGTGAGAACGGTATTACTTATGCTTCTGCTTGTTTGGCTGGTTGTCA |
| | AACTTCTACAAGATCTGGTAAGACCACCATTTTCAACAACTGTACATGTGTTGGTTTGGCTGC |
| | TTCTAAGTCTGGTAATTCTTCTGGTATGGTCGGTAGATGTCAAAAGGATAATGGTTGTGCTA |
| | AGATGTTCCTGTACTTCTTGATTATTGCCGTCATTACCTCCTACACTTTGTCTGTTGGTGGTAT |
| | TCCAGGTTACATCTTGTTGTTGAGATGCATCAAACCACAGTTGAAGTCTTTCGCTTTGGGTAT |
| | CTATACTTTGGCCGTTAGAGTTTTGGCAGGTATTCCATCTCCAATCTATTTCGGTGTTTTGATC |
| | GATACCTCTTGTTTGAAGTGGGGTTTTAAGAGATGTGGTACTAAGGGTTCTTGTAGGTTGTA |
| | CGATTCTAATGCCTTCAGACATATCTACCTGGGTTTGACTATGATGTTGGGTATCATGTCCAT |
| | CTTTTTGTCTACCGCTGTTTTGTTCACCCTGAAGAAGAACTACGTTTCTAAGCACAGATCCTTC |
| | AGAACTAAGAGGGAAAGAACTATGGTTTCCACCAGGATCAGAAAAGAAAACTGTACTACCA |
| | ACGATCACTTGTTGCAACCTAATTATTGGCCAGGTAAAGAAACCCAGTTGTAA |
| SEQ ID | MDTSSKENIRLFSKNSMQPVGRLSFKTEYPSSEEKQPCCCQLKVFLGALSFVYFAKALAEGYLKSTI |
| NO: 922 | TQIERRFDIPSSLVGIIDGSFEIGNLLVIAFVSYFGAKLHRPKIIGAGCLIMGVGTLLIATPHFFMEQY |
| | KYERYSPASNSTLSISPCLLDSNNSLPIPAVEKSQSKINNECQVDASSSMWVYVFLGNLLRGFGET |
| | PIQPLGIAYLDDFASQDNAAFYIGCVQTVAIIGPIFGFLLGSLCAKLYVDTGFVNLDHITITPKDPQ |
| | WVGAWWLGYLIAGSITLLAAVPFWCLPRTLPRPRSREDSSSSSEKSKFIMDDLTDYQTPPGEKAK |
| | IMEMARDFLPSLKSLFGNPVYFLYLCASTVQFNSLFGMVTYKPKYIEQQYGQSASKANFVIGLINIP |
| | AVALGIFSGGIIMKKFRVSIRGAAKLYLGSSVLGYLLFLSLFALGCENSDVAGLTISYQGTKPVSYHE |
| | RALFSDCNSRCKCSETKWEPICGENGITYASACLAGCQTSTRSGKTTIFNNCTCVGLAASKSGNSS |
| | GMVGRCQKDNGCAKMFLYFLIIAVITSYTLSVGGIPGYILLLRCIKPQLKSFALGIYTLAVRVLAGIPS |
| | PIYFGVLIDTSCLKWGFKRCGTKGSCRLYDSNAFRHIYLGLTMMLGIMSIFLSTAVLFTLKKNYVSK |
| | HRSFRTKRERTMVSTRIRKENCTTNDHLLQPNYWPGKETQL |
| SEQ ID | ATGGCTGCCTTGGATAGAGAAAGATTGAGAAGATTATTGCCCTACTTGGGTAGAGATAGAA |
| NO: 923 | GAAGGTTGTTGGTTACCTTGGTTTTGTTGATTCCAGTTGCTGGTGCTGCTGCTATTCAACCAT |
| | TATTGGTTGGTCAAGTTATCGCCGTTTTGAGAGGTGAACCTACTTTGACATTTTTGACTGGTA |
| | TGCCAGTTCCACAAGCCTTGAGATTATTAGTTTTGGTTTTATTGGGTGCCGTCTTGTTGAGAT |
| | TGGCTTTACAAGGTTTACAGTCCTACTCTGTTCAAGCTGTTGGTCAAAGATTGACCGCTAGAA |
| | TTAGAGATGATTTGTTCGCTCATGCTATGGCCTTGTCTTTGAGATTTCATGATAGAACTCCAG |
| | TCGGTAAGTTGTTGACTAGATTGACTTCTGATGTTGATGCTTTGGCTGAAGTTTTTGGTTCTG |
| | GTGCTGTTGGTGTTTTAGCTGATTTGGTTACTTTGTTGGTCATTGCCTTGACCATGTTGTCTAT |
| | TGAATGGCGTTTGGGTTTGCTGTTGTTGTGTTCTCAAGTTCCAGTTGTTTTGGGTATGTTGTG |
| | GTTGCAAAGAAGATACAGAAGGGCTAACTACAGAGTCAGAGAAGAATTGTCTCAATTGAAC |
| | GCTGACTTGCAAGAAAACTTGCAAGGTTTGGAAGTTGTCCAGATGTTCAGAAGAGAAAGAG |
| | TTAATTCTGCTAGATTCGCTAGAACTACCGATGCTTATAGACAAGCTGTTACCGGTACTATCT |
| | TTTACGATTCTGCTATTTCCGCCTTCATTGAATGGGTTGCTTTGGTTGCTGTTGCATTGGTTTT |
| | GGCTTTAGGTGGTTCTATGGTTACAGCTGGTGCTTTAGGTTTGGGTACTTTGACTACTTTCAT |
| | CTTGTACTCCCAAAGGTTGTTCGATCCATTAAGACAATTGGCTGAAAGATTCACCCAAATCCA |
| | AGGTGGTTTGACTGCTGTTGAAAGAATCGGTGAATTATTGGAACAGCCAATCGAAATCCAAG |
| | AATTGCCAGCTTCTCAAAGATCTGCTGCAGCTAGAAGATCTGGTGGACAAAGATCATCTGCT |
| | GGTGAAGTTGTTTTCGAACATGTTTCTTTCGCCTACAGACAAGATGATCCAATTTTGACCGAT |
| | CTGTCCTTTAGAATTGCTCCAGGTGAACATGTTGCATTAGTTGGTCCAACAGGTTCTGGTAAG |
| | ACTACTGTTATTAGATTGCTGTGCAGGTTGTACGAACCTCAACAAGGTAGAATTTTGTTGGAT |
| | GGTATCGACATTAGGGAATTGCCTATTCCAACTTTGAGACAAAGATTGGGTGTTGTATTGCA |
| | AGACACCTTTTTGTTCTCTGGTAACGTTGCCGATAATTTGAGATTAGATGCTCCAATTGCCAA |
| | CGATGAATTGGCCCAATTGTGTAGAGAATTGGGTTTAGATCCTCTGCTAAGAAGATTGCCAG |
| | AAGGTTTGGCTACTGAATTGAGAGAACGTGGTGGTAATTTGTCATCTGGTGAAAGACAATTA |
| | TTGGCCGTTGCTAGAGTTGCTATTAGAGATCCATCTGTTTTGGTTATGGATGAAGCTACTGCA |
| | TTTTTGGATCCATCTACTGAAGCTACCTTGCAACAAGATTTGGATAGATTGCTACAACAAAGA |
| | ACCGCTATCGTTATTGCTCATAGATTGGCAACTGTTGAAGCTGCTGATAGAATATTGGTATTG |
| | CAGAGAGGTAGATTGATCGAACAGGGTACTCATAGACAATTGAGAGCTGCTGGTGGTCTAT |
| | ATGCTAGATTAGCTGAATTACAGGATAAGGGTTTAGCTGCCTTGTGA |
| SEQ ID | MAALDRERLRRLLPYLGRDRRRLLVTLVLLIPVAGAAAIQPLLVGQVIAVLRGEPTLTFLTGMPVP |
| NO: 924 | QALRLLVLVLLGAVLLRLALQGLQSYSVQAVGQRLTARIRDDLFAHAMALSLRFHDRTPVGKLLTR |
| | LTSDVDALAEVFGSGAVGVLADLVTLLVIALTMLSIEWRLGLLLLCSQVPVVLGMLWLQRRYRRA |
| | NYRVREELSQLNADLQENLQGLEVVQMFRRERVNSARFARTTDAYRQAVTGTIFYDSAISAFIE |
| | WVALVAVALVLALGGSMVTAGALGLGTLTTFILYSQRLFDPLRQLAERFTQIQGGLTAVERIGELL |
| | EQPIEIQELPASQRSAAARRSGGQRSSAGEVVFEHVSFAYRQDDPILTDLSFRIAPGEHVALVGPT |
| | GSGKTTVIRLLCRLYEPQQGRILLDGIDIRELPIPTLRQRLGVVLQDTFLFSGNVADNLRLDAPIAND |
| | ELAQLCRELGLDPLLRRLPEGLATELRERGGNLSSGERQLLAVARVAIRDPSVLVMDEATAFLDPS |
| | TEATLQQDLDRLLQQRTAIVIAHRLATVEAADRILVLQRGRLIEQGTHRQLRAAGGLYARLAELQ |
| | DKGLAAL |
| SEQ ID | ATGAACTCCGTTGAATCCGCCGATAAGAACTATGTTATAGTTTCCGATGGTTCCGAGAAGTT |
| NO: 925 | GTCTCCAGTTCCACCATTGCAAAGAAGATTATTGACTCCATTCCTGTCCAAAGAAGTTCCACC |
| | TATTCCAGTTGATGAAGAAAGAGTTAAGTACCCCTACTTGATGACTAACCCATTCTCTACTGT |
| | TTTCTTCACTTGGTTGAACCCTTTGTTGAAGGTTGGTTACAAGAGAACTTTGTCCCCAAACGA |
| | TTTGTTCAAGATTGCTGATAAGGATGCCTTGGATTACACTTACTCTACCTTCGAAAGACACTT |
| | GGACGAAATCATCCAAAAGGATAGAGATGCCTTGAAGTTGAGAGATCCATCTATTACCGAA |
| | GAAGAATTGGAAGCTAGAGAGTATCCAAAGAACGCTATTTTGAAGGCCTTGCTGAAAACTTT |
| | CAAGTGGGAATATTCTTTCGCCGTGTTCTTCAAGTTGTTGTCTGATACTGCTCAAGTTTTGACT |
| | CCCTTGTTGTCTAGAGCTTTGATTACTTACGTTCAAGACAAGACTGTTGATCCAGGTTTGCCA |
| | GTTAACAATGGTGTTGGTTATGCTATTGGTGTCACTTTCATGTTGGCTGTTACTACTTTGTGTA |
| | CCAACCACTTCTTGTACTTGTCTTTGACCGTTGGTTTACATGCCAAGTCTATTTTGACTACCGC |
| | TGTCTTGAAGAAGTCTTTCAAAGCTTCATCTGTTACCAGACATACTTTCACCAATGGTAGAGT |
| | TACCTCCTTGATTTCTGCTGATTTGGCTAGAGTTGATTTGGCCTTTGGTTTTCAACCATACGTT |
| | ATTACTGCACCCGTTCCAATTATCGTTACCATTGCTTTGCTGATCATCAACATCGGTGTTTCTT |
| | CATTGGCTGGTATTGCTATGTTTTTGGTTGCCTTGTTTGTTATTGGTGCTTGTTCTGGTGCTTT |
| | GATGGCTTTGAGAAAGAAGGTTAACAAGTTCACCGACTCCAGAATCTCTTACATGAGAGAAA |
| | TCTTGCAGAACATGAAGATCATCAAGCTGTACTCTTGGGAAGATGCTTACGAAAAGACTGTT |
| | ACTGACGAAAGAAACAACGAAATCGGTGTCATGTTGAAGATGGTGTCTGTTAGAAACTTTTT |
| | GATGGCCTTCGCTATCTCTTTGCCATCTGTTGTTTCTATGGTTGCTTTCTTGGTCTTGTACGGT |
| | GTTTCCCAAGATCAAAATCCAGCCAATATTTTCACCTCCTTGTACCTGTTTAGTTTGTTGGCTG |
| | GTCAAATCATGATGGTTCCAATGGCTTTGTCTACTGCTACTGATGCTAAAATCGGTTTGGAAA |
| | GATTGAGGTTGTACTTGCAATCTGGTGAAGTTCAATCTAACGATGATGACAAAGGTGAAGAT |
| | GGCACTAATTCTTTGCCAGAAGATGTTGCTATTCAAGTTACCAACGCTTCCTTCATTTGGGAA |
| | AAGTTCGAAGATGAAGATAACGAAGCTGAAGAAACTCCAGAAACCACCTCTTCTATTGAAAG |
| | ATTGGACGAATTGTCTAAGCACGACTTTGAAGGTTTCTCCAACATCAACTTCTCCATCAAGAA |
| | GTCCGAAGTTGTTTTGATTACAGGTCCAATCGGTTCCGGTAAATCATCTTTGTTGTTGGCTTT |
| | AGCAGGTCAAATGACTAAGACTTCCGGTTCTTTGAAAACTGCTGGTTCTTTGTTATCTGCTGG |
| | TCAACCATGGATTCAAAACGCTACTGTCAAAGAAAACATCTTGTTCGGTGAACCATTCAACCA |
| | GGATAGATACCAACAAGTTGTTAGAGTTTGCGCTTTGGAAGATGACTTGCAGTCTTTTACTG |
| | CTGGTGATGCTACTGAAGTAGGTGAAAGAGGTATTACTTTGTCCGGTGGTCAAAAGGCTAG |
| | AATCAATTTGGCAAGAGCTGTTTACGCTAACAAGGACATTATCTTGTTGGATGATGTTTTGTC |
| | CGCTGTCGATTCTAGAGTTGGTAAGATTATCGTTGACGAGTGCTTCAAGAAGTTCTTGCGTA |
| | AAAAGACTATCGTTTTGGCTACCCACCAATTGTCTTTGGTTCATTTTGCTGATAGAGCCATCTT |
| | CTTGAACGGTGATGGTTCTATTAACATCGGTACTGTCGAAGAATTGTCCAAAACTTCTTCTGG |
| | CTTCCTGAAGCTAATGGAATACTCTTCTAGACCAGATTCCGATACCTCTAATGATGGTCAAAA |
| | CGAAAAGTTGGAGGGTATCTTGTCAGGTAAGTCTGTTCATCATAACCCACAAGAATCCACCT |
| | TGATTGAAGATGAGGAAAGAGCAGTTAACGCCATTGAATGGTCTGTTTACAAAGCCTACTTG |
| | CATGAAGGTCAAGGTAAATTTGGTGTTTTCGCCATTCCATTGATTGCCATGTTTATGACCTTT |
| | GACGTTTTCTTGACCATCTTCGTTAATGTCTGGTTGACCTACTGGATTAACGATTCATTTCCAG |
| | GTAGATCTGACGGTTTCTACATTGGTTTTTACGTTATGTTCGTCGTCTTGTCTATCGTTGCTAT |
| | CTCCTCTGAATTCATTATGATGGGCTACTTTTTCAGCACTGCATCTAGAAGGTTATACCTGAA |
| | GGTTTTGAAAAGAGTCTTGCATACCCCAATGCACTACTTGGATGTTACTCCAATGGGTTGTGT |
| | TTTGAACAGATTCACTAAGGATACCGACTCATTGGATAACGAAATTGGCCAAGAATTGACCA |
| | TGTTGGTTTATCCAGGTGCAATTTTGACCGGTACTGTTATTTTGTGCGTTGTTTACTTGCCATG |
| | GTTCGCTATTGCTTTACCACCATTATTGGTTGTTACCACTGCTACTACCAATTTCTACGTTGCTT |
| | CTTCCAGAGAAGTCAAGAGAATTGAAGCCTTGCAAAGATCCCATGTGTTCAACAACTTACAC |
| | GAAATTTTGAACGGCTTGCAAACCTTGAAAGCTTACAATGCTTTGGAAAGGTTCATGGACAA |
| | AAACAAGCACTTGATCGACAGAATGAACGAAGCCTACATTTTGGTTATCGCCAATCAGAGAT |
| | GGATCAGCATTCATTTGGAACTGGTTTCTTGCTTGTTGGTTTTGGTTGTTGCTCTGTTGTCTGT |
| | GTTCAGAGTGTTCAATATTAACGCTGCTTCTGTCGGCTTGATTATCAACTACGTTATGCAAGT |
| | TGCTACCTTCATGTCCTTGGTTATGAGATCTTACACCTCTGTCGAAAACGAGATGAATTCTGT |
| | TGAAAGGTTGTGCTTCTACGCCCATGATTTGGAACAAGAACCACCATATAGAATCAACGAAA |
| | CTAGACCAAGACCATCTTGGCCAGAACATGGTGCAATAGAATTTCAGAACGTGTCCATGAGA |
| | TACAGATATGGTTCTCCATTGGTCTTGAGGAACTTGTCCATTTCTGTAAAAGGTGGTGAAAA |
| | GATCGGTATTTGTGGTAGAACTGGTGCTGGTAAATCCTCCATTATGAACGTCTTGTTCAGATT |
| | GTCAGAACCAGCTGAAGGTACTGTTTTGATCGATGGTGTTGATACTTCTAAGTTGGGCTTGT |
| | ACGATTTGAGGTCCAAGTTGTCTATTATTCCACAAGATCCAGTGTTGTTCAGGGGTACTATTA |
| | GAAAGAATTTGGACCCATTCGAACAGTCCGAAGATTCTGCTTTGTGGGAAGCTTTAAGAAAG |
| | GCTGGTTTGATTGATACCGACGTTTTGAGAAAAGCCATTTCTCAACATCCAGATGATCCAAAC |
| | AGACACAAGTTCCATTTGGATCAATTGGTTGAGGATGACGGTTCTAATTTCTCTAATGGTGA |
| | AAGACAGTTGATCGCTTTAGCTAGAGCATTGGTTAGAGACTCCAAGATCTTAGTTTTAGATG |
| | AAGCCACCTCCTCCGTTGATTACGAAACAGATGCAAAAATCCAGAACACTATCGCCGAAGAA |
| | TTCTCTGAATGTACCATTTTGTGCATTGCCCACAGATTGAACACCATTTTGAACTACGATAGG |
| | ATCCTGGTCTTAGAACAAGGTAAGGTTGAACAATTTGATACCCCATGGGCTTTGTTTAACCA |
| | GGATGGTATTTTCAGACAGATGTGCGAAAGATCTGGTATCAAGTCATCAGAAAGATTGTCTC |
| | AGAACTGGTCCTTGAGATTGGATGGTGCTTAA |
| SEQ ID | MNSVESADKNYVIVSDGSEKLSPVPPLQRRLLTPFLSKEVPPIPVDEERVKYPYLMTNPFSTVFFT |
| NO: 926 | WLNPLLKVGYKRTLSPNDLFKIADKDALDYTYSTFERHLDEIIQKDRDALKLRDPSITEEELEAREYP |
| | KNAILKALLKTFKWEYSFAVFFKLLSDTAQVLTPLLSRALITYVQDKTVDPGLPVNNGVGYAIGVTF |
| | MLAVTTLCTNHFLYLSLTVGLHAKSILTTAVLKKSFKASSVTRHTFTNGRVTSLISADLARVDLAFGF |
| | QPYVITAPVPIIVTIALLIINIGVSSLAGIAMFLVALFVIGACSGALMALRKKVNKFTDSRISYMREIL |
| | QNMKIIKLYSWEDAYEKTVTDERNNEIGVMLKMVSVRNFLMAFAISLPSVVSMVAFLVLYGVSQ |
| | DQNPANIFTSLYLFSLLAGQIMMVPMALSTATDAKIGLERLRLYLQSGEVQSNDDDKGEDGTNSL |
| | PEDVAIQVTNASFIWEKFEDEDNEAEETPETTSSIERLDELSKHDFEGFSNINFSIKKSEVVLITGPIG |
| | SGKSSLLLALAGQMTKTSGSLKTAGSLLSAGQPWIQNATVKENILFGEPFNQDRYQQVVRVCAL |
| | EDDLQSFTAGDATEVGERGITLSGGQKARINLARAVYANKDIILLDDVLSAVDSRVGKIIVDECFKK |
| | FLRKKTIVLATHQLSLVHFADRAIFLNGDGSINIGTVEELSKTSSGFLKLMEYSSRPDSDTSNDGQN |
| | EKLEGILSGKSVHHNPQESTLIEDEERAVNAIEWSVYKAYLHEGQGKFGVFAIPLIAMFMTFDVFL |
| | TIFVNVWLTYWINDSFPGRSDGFYIGFYVMFVVLSIVAISSEFIMMGYFFSTASRRLYLKVLKRVLH |
| | TPMHYLDVTPMGCVLNRFTKDTDSLDNEIGQELTMLVYPGAILTGTVILCVVYLPWFAIALPPLLV |
| | VTTATTNFYVASSREVKRIEALQRSHVFNNLHEILNGLQTLKAYNALERFMDKNKHLIDRMNEAYI |
| | LVIANQRWISIHLELVSCLLVLVVALLSVFRVFNINAASVGLIINYVMQVATFMSLVMRSYTSVENE |
| | MNSVERLCFYAHDLEQEPPYRINETRPRPSWPEHGAIEFQNVSMRYRYGSPLVLRNLSISVKGGE |
| | KIGICGRTGAGKSSIMNVLFRLSEPAEGTVLIDGVDTSKLGLYDLRSKLSIIPQDPVLFRGTIRKNLD |
| | PFEQSEDSALWEALRKAGLIDTDVLRKAISQHPDDPNRHKFHLDQLVEDDGSNFSNGERQLIALA |
| | RALVRDSKILVLDEATSSVDYETDAKIQNTIAEEFSECTILCIAHRLNTILNYDRILVLEQGKVEQFDT |
| | PWALFNQDGIFRQMCERSGIKSSERLSQNWSLRLDGA |
| SEQ ID | ATGACCTCCAAAGAAACCTCTATGGGCTTGCAAGTTGATGATGATTCTCCATTGCCAGAATTG |
| NO: 927 | GAGAGAAGATTGATGACTCCATTCTTGTCTAAAACGGTTCCACCAATTCCAAAAGAAAACGA |
| | AAGATTGCCATACCAATTGAACCATGCCAACTTCATCTCCAAGATATTTTTCTGGTGGCTGAA |
| | CCCAATTATGAACGTTGGTTACAAGAGAACTTTGACCCCAAATGACTTGTACTACGTTGAAAA |
| | CGATTTGACCGCTAAGCAAAACTACGATGACTTCATGATCAACCTGAACAAGGTTTTGTCTAA |
| | GGCTAAATTGAAGGCCAAGTTGAAGAACCCAGAATTGACCGATGATGAATTGGACGAATTG |
| | CCTTATCCTAAGTTCTCCTTGGTTATTGCTTTGCTGATGACCTTCAAGTACAAGTACTCTAAGG |
| | CTGTGTTCTTCAAGTTGTTGTCCGATATCTCTCAAGTCTTGAATCCTTTGTTGACCAAGGCTTT |
| | GATTAACTTCGTTGAGAAAAAGACCTACTTGCCAGATACTCCAGTTGGTAAAGGTATTGGTT |
| | ATGCCATTGGTGTTTCTGCTATGTTGGTTGCTAATGGCATTTTCCTGAACCATTTCTTGCACAA |
| | CTCTATGAATGTTGGTGCCCAATCCAAGTCTATTTTGACTACTGCTTTGTTGAAGAAGTCCTTC |
| | AAAGCTACTCCAAAGACTAAGCACACTTACACCTCTGGTAAGGTTACCTCTATTTTGTCCACA |
| | GACTTGTCCAGAATCGATTTGGCTATTGGTTTTCAACCATTCGCCATTACTTTTCCAGTTCCAG |
| | TTATAGTTTCTGTCGCCTTGTTGATCGTTAACATAGGTGTTTCAGCTTTGGTTGGTATTGCCAT |
| | TTTCTTGGTTGCTGTTGGTGGTATTGGTACTTCTGCTAAATGGTTGTTGAACATGAGAAGAG |
| | GTGCTAATCAGTACACTGATAAGCGTGTTGGTTTGATGAGAGAAATCCTGAACTCCATGAAG |
| | ATCATCAAGTTCTACTCTTGGGAAGATGCCTACGAAAAGAACGTTGTTGATCAGAGGAACAA |
| | AGAGATCTCCATCATCTTGAAGATGCAGACGATTAGGAATTTCTTGTTCGCTTTCGCTATTTC |
| | CTTGCCAACCGTTATTTCTATGGTTTCCTTTTTGGTCTTGTACGCCTTGTCTAACAAACAAAAT |
| | GCCGGTGATATCTTCTCCTCCTTGTCTTTGTTTGGTACTTTGTCCCAACAGGTTTTGATGTTGC |
| | CAATGGCTTTAGCTACTGGTGCTGATGCTTTTATTGGTATCACTAGAGTCAGAGAATACTTGC |
| | AGACTAAGGATTTGGAAGTCGAAGATGGTATTCCAGACAACTACAACAACGAAAACTTGGA |
| | CGAAAATCTGGCCTTGAAAGTTACTGATGCTTCTTACAAGTGGGAAAAGTTCGACGATATCG |
| | AAGATGAAGAAAAGCAACAACAAGAAGCTGCCGAAAACGAAGATGAGGATGATGATAACG |
| | AAACGAAGTTGGAGTCTAACAAGAACGAGTCCTCTATCTCTTCTGAGTTTTCTTCAAAGGACG |
| | AAAAGACTTCTACCAAGGCTGTTTTTGAAGGTTTCAACAACATCAACCTGGAAATCAAGAAG |
| | AATGAGTTCGTCATTATTACCGGTCCAATCGGTACTGGTAAATCCTCTTTGTTAACTGCCTTGT |
| | CCGGTTTTATGAAGCAAACCTCTGGTGAAATTGCCATCAATGGTTCTTTGTTGTTGTGTGGTC |
| | AACCATGGGTTCAAAACGCTACTTTCAAAGAGAACATCATCTTCGGTTCTCCATACGAAGAA |
| | GAAAGATACAACAAGGTTATTGAAGTCTGCGCCTTGAAGGATGATTTGAAAATTTTGCCAGG |
| | TGGTCACAACTCCGAAATTGGTGAAAGAGGTATTAACTTGTCTGGTGGTCAAAAGGCTAGAA |
| | TCAACTTAGCTAGAGCTGTTTACTCTGGTAAAGACACCATTTTGTTCGATGATGTTTTGTCCG |
| | CTGTTGATGCTAGAGTCGGTAAACATATTATCGATGAATGCTTCACCAAGTTCTTGGCTGATA |
| | AGACTAGAATTTTGGCTACCCACCAATTGTCCTTGATTGATAAGGCTGATAGAGTCATCTTCT |
| | TGAACGGTGATGGTACTATTAACATTGGTACTGTCCCAGAGTTGCTGTCCTCTAACAAAAATT |
| | TCGAAAAGTTGATGGACTTCGCCACCTCTTATAATGATCCAGACTTAGAGGATGAGGTCTTG |
| | ATCAAAGAAGATAACCAGTTGCAAAGGCAGCTGTCTAAGAAGTTCACTGAATCCAAAGAATT |
| | CGAGGACCCAGAAAATGAGAACGGTGTTTTGATAGCTGATGAAGAGAGAGCTAGAAACGCT |
| | ATTTCTTGGTCTGTTTACAAGCAGTTCTTGAAAGAAGGTCAAGGTATGTTTGGTGTTTTCGCT |
| | GTTCCATTGGCATTCTTGTTGATGGTTGGTGATGTTTTCTGCTCCATTTTCGTTAACGTTTGGT |
| | TGTCCTTCTGGATCTCTAAAAAGTTTCCAGGTAGATCCGATGGTTTCTACATTGGTATCTACG |
| | TGATGTTCACCTTCACCTCTGTTTTGATGATTTCCTGCGAATTCATGGTCATGGGTTACATTAC |
| | TACTACCGCCTCTAAGAAGCTAAACTTGAGAGCTATGGTTAGAGTCTTGCATACTCCAATGTC |
| | TTTCTTGGATACAACCCCAATTGGTAGGGTCTTGAATAGATTCTCTAAGGATACCGATGTCTT |
| | GGACAATGAGATAGGTGGTCAATTGAGAATGTTTACTCATCCAGCCGGTTTTGTTATCGGTG |
| | TCATTATTTTGAACATCGTCTACTTGCCTTGGTTTGCTTTGTCTATTCCACCATTGGCCATTTGC |
| | TTCGTGTGTATTACTAATTACTACCAGTCCTCCAGCAGAGAGATTAAGAGATTGGAAGCTGTT |
| | CAAAGGTCCTTCGTCTACAACAATTTTAACGAAGTGTTGTCCGGTATGAACACCATTAAGGCT |
| | TACAAATCCAACAACAGGTTCATCGTTAAGAACGACTACTTGATCGACAGAATGAACGAAGC |
| | TTACTACATCACCATTGCCAATCAAAGATGGATCTCCTTGCATTTGGATTTGGTTGCTTGTGCT |
| | TTGGCTTTGGTCATTACAATTTTGTCTGCCACTAGACAGTTCGATATTAACGCTGCTTCTGTCG |
| | GTTTGATTACTACCTACACTTTACAATTGGCCGGCTTGATGTCTTTGATTTTGAGAGCTTACAC |
| | CCAGATCGAGAACGAAATGAATTCTGTTGAAAGATTGTGCCACTACGCTAATGATTTGGCTC |
| | AAGAAAACCAGTACAGAAACGAAGAAACAAAGCCACAAGGTCAATGGCCAACTCAAGGTGC |
| | TATTGAGTTTAATCAAGTCTCCTTGAGATACAGAGATGGTTTGCCTTTGGTTCTGAAGAACTT |
| | GTCCATCAATATTGGTGGTGGTGAAAAAGTTGGTATTTGTGGTAGAACAGGTGCCGGTAAA |
| | TCTTCTATTATGGTTGCCTTGTACCGTCTGAACGAATTGTCTCAAGGTACTATCAAAATCGAT |
| | GGTGTCGATGTTTCTAAGTTGGGTTTGTTTGACTTGAGGTCCAAGTTGTCCATTATTCCACAA |
| | GATCCAGCTTTGTTCCAAGGCTCTATTAGAAAGAATTTGGACCCATTCGATGAACACGAGGA |
| | TTCAATTTTGTGGGATGCTTTGAGAAGATCCGGTTTGATAGAACCAGAAGAATTGTCCTCTG |
| | CCATTAACAACTCTGATAAGTCTACCTTCCATAAGTTCCACTTGGATCAATTGGTTGAGGATG |
| | AAGGTGCTAACTTTTCATTGGGTGAAAGACAATTGATTGCTTTGGCAAGAGCTTTAGTTAGA |
| | GACTGCAAGATTTTGATCTTGGATGAAGCTACCTCCTCCGTTGATTACGAAACTGATTCTAAG |
| | ATCCAAAACACCATCAAGAACGAATTCAAGGATTGCACCATCTTGTGCATTGCCCATAGATTG |
| | AAAACTATCTTGAACTACGACAAGATCCTGGTCTTGGAAAAAGGTGAAATCGAACAATTTGA |
| | CGAGCCCATTAAGTTGTTCCACGATGAATTAGGTATCTTCAAGCAGATGTGCGAAAAGTCCG |
| | ATATTACCATTGCTGACTTCGAGTGA |
| SEQ ID | MTSKETSMGLQVDDDSPLPELERRLMTPFLSKTVPPIPKENERLPYQLNHANFISKIFFWWLNPI |
| NO: 928 | MNVGYKRTLTPNDLYYVENDLTAKQNYDDFMINLNKVLSKAKLKAKLKNPELTDDELDELPYPKF |
| | SLVIALLMTFKYKYSKAVFFKLLSDISQVLNPLLTKALINFVEKKTYLPDTPVGKGIGYAIGVSAMLV |
| | ANGIFLNHFLHNSMNVGAQSKSILTTALLKKSFKATPKTKHTYTSGKVTSILSTDLSRIDLAIGFQPF |
| | AITFPVPVIVSVALLIVNIGVSALVGIAIFLVAVGGIGTSAKWLLNMRRGANQYTDKRVGLMREIL |
| | NSMKIIKFYSWEDAYEKNVVDQRNKEISIILKMQTIRNFLFAFAISLPTVISMVSFLVLYALSNKQN |
| | AGDIFSSLSLFGTLSQQVLMLPMALATGADAFIGITRVREYLQTKDLEVEDGIPDNYNNENLDENL |
| | ALKVTDASYKWEKFDDIEDEEKQQQEAAENEDEDDDNETKLESNKNESSISSEFSSKDEKTSTKA |
| | VFEGFNNINLEIKKNEFVIITGPIGTGKSSLLTALSGFMKQTSGEIAINGSLLLCGQPWVQNATFKE |
| | NIIFGSPYEEERYNKVIEVCALKDDLKILPGGHNSEIGERGINLSGGQKARINLARAVYSGKDTILFD |
| | DVLSAVDARVGKHIIDECFTKFLADKTRILATHQLSLIDKADRVIFLNGDGTINIGTVPELLSSNKNF |
| | EKLMDFATSYNDPDLEDEVLIKEDNQLQRQLSKKFTESKEFEDPENENGVLIADEERARNAISWS |
| | VYKQFLKEGQGMFGVFAVPLAFLLMVGDVFCSIFVNVWLSFWISKKFPGRSDGFYIGIYVMFTFT |
| | SVLMISCEFMVMGYITTTASKKLNLRAMVRVLHTPMSFLDTTPIGRVLNRFSKDTDVLDNEIGGQ |
| | LRMFTHPAGFVIGVIILNIVYLPWFALSIPPLAICFVCITNYYQSSSREIKRLEAVQRSFVYNNFNEVL |
| | SGMNTIKAYKSNNRFIVKNDYLIDRMNEAYYITIANQRWISLHLDLVACALALVITILSATRQFDIN |
| | AASVGLITTYTLQLAGLMSLILRAYTQIENEMNSVERLCHYANDLAQENQYRNEETKPQGQWPT |
| | QGAIEFNQVSLRYRDGLPLVLKNLSINIGGGEKVGICGRTGAGKSSIMVALYRLNELSQGTIKIDGV |
| | DVSKLGLFDLRSKLSIIPQDPALFQGSIRKNLDPFDEHEDSILWDALRRSGLIEPEELSSAINNSDKST |
| | FHKFHLDQLVEDEGANFSLGERQLIALARALVRDCKILILDEATSSVDYETDSKIQNTIKNEFKDCTI |
| | LCIAHRLKTILNYDKILVLEKGEIEQFDEPIKLFHDELGIFKQMCEKSDITIADFE |
| SEQ ID | ATGGCCGAAGATTTGGGTTTGTCCTCTCCAAAAACTGATCCAGAAGATTCCTCTAAGTTGGTC |
| NO: 929 | TTGGAGAGAAGAATTATGACCCCATTCTTGTCTAAAAAGGTTCCACCAGTTCCAACTGAAGCT |
| | GAAAGAAAGTATTTCCCCAAGAACAAGAACCCCTTGTCCTTGATTTTCTACTGGTGGTTGAAT |
| | CCAATCATGAAGGTTGGTTACAAGAGAACTTTGACTCCAAACGACTTGTACAAATTGACCCC |
| | AGAAATGAAGATCGATCACACCTATGATAAGTTCGAGAAGATCCTGATGAAGATTGTCGAA |
| | AAGGATAGAGCTAAAGCCTTGGCTGAAGATCCATCTTTGACTGAAGAAGATTTGATCAGAA |
| | GGCCCTATCCAAAGTATGCTTTGCCAAAAGCTTTGTTCTTGACCTTCAAGTGGAAGTACATTT |
| | TGGCCTTGTTCTTCAAGGTTTTGGCTGATGTTTGTGGTGTCTGTAATCCTTTGTTGTCCAAGA |
| | AGTTGATTGCCTTCGTTGAGAGAAAAACCTCTGATCCATCATTGGCTGTTAACGATGGTATTG |
| | GTTATGCTTTGGGTTGTACCTTCTTGGTTTTGTTCTCTGGCATCATGATTAACCAGTCCTTGTT |
| | GCATTCTTTGACTACTGGTGCTCATTGCAAGGGTATTTTAACTACTGCTTTGCTGCGTAAGTCT |
| | TTCAAAGCTGATGCTGAAACTAGACACAAGTACACCTCTGGTAGAATTACCTCTATTATGTCT |
| | ACCGATTTGGCCAGAATTGATTTGGCTATTGGTTTTCAACCATTCGGCTTGACTTTTCCAATTC |
| | CAGTTATTATTGCCATAGCCTTGTTGATCGTTAATATCGGTGTTTCTTCTTTGGCTGGTATTGC |
| | CGTTTTCATCATTTCCATAGTTGCTATTGGTGGTTCCGCTAGATTCCTAATGAAGATGAGAAG |
| | AGGTGCTAACAAGTATACCGACAAGAGAATCTCTTTGATGAGGGAAATCTTGCAGTCCATGA |
| | AGATGATCAAGTACTACTCTTGGGAAGATGCCTACGAAAAGTCCGTTATTCAGCAAAGAACA |
| | AAAGAGGTCGGTATCATCTTGAAGATGCAGTCTATTAGAAACGGTTTGTTGGCTTTCTCTATT |
| | GCCTTGCCAGCTTTCACTTCTATGATTGCATTTTTGGTCTTGTACGGCGTGTCCTCTAACAAAA |
| | ATCCAGCTAACATTTTCCCCAGCGTTTCTTTGTTTGGTACTTTGGCTCAACAAACCATGATGTT |
| | GCCAATGGCTTTGGCTACAGGTGCTGATGCTATGATTGGTTTGGGTAGAGTTAGAGAATTCT |
| | TGGAATCTGGTGTTGATTTGAAGGACCCTGAAGAATTTGATGGTCATGCTCCAGAAACAGAC |
| | GAAGAGGTAAAAGAATTGCCATCCGATGTTGCTATCGAAGCTAAAGATGCTACTTTCATCTG |
| | GGAAAAGTTCGCTGAAGTTGACTCTGAAGAAACTTCTGATGCTTCCAAGTCTGACAAAGAGA |
| | CTTCTTCTACTTTGTCCGAAGGTGAGTTGAAAAAGACCTTGTCATCTGAAGAGGAAGAAGGT |
| | AACGAACAGTACACTAACTCTGTTTTCGAAGGTTTCCACAACATCAACTTGGAGATTAAGAA |
| | GAACGAGTTCATCATTGTCACTGGTGCAATTGGTTCTGGTAAGTCATCTTTGTTGACTGCTTT |
| | AGCTGGTTTCATGAAGAGAACTACTGGTTCTTTGTCTGTTGGTGGTAGTGTTTTGTTGTGTGG |
| | TACTCCATGGGTTCAAAATGCTACTGTTAGAGAAAACATTACCTTCGGTTTGGAATACGACG |
| | AAGAAAGATACGAAAGGGTTTTAGATGCTTGCGCTTTGAGAGATGACTTGAAGTTGTTTACA |
| | GGTGGTGATTTGACCGAAATTGGTGAAAGAGGTATTACATTGTCTGGTGGTCAAAAGGCTA |
| | GAATCAATTTGGCTAGAGCTGTTTATGCCGACAAAGAAATCGTTTTGTTCGATGATGTTTTGT |
| | CCGCTGTTGATGCTAGAGTTGGTAAACATATAGTTGATGATTGCCTGTGCGATTTCATGGGT |
| | CATAAGACTAGAATTTTGGCTACCCACCAATTGTCTTTGATTGATAAGGCTGATAGGGTCATC |
| | TTCTTGAATGGTGATGGTACTATTCACATCGGTACAGTTAATGAGTTGTTGCAGTCTAATGAA |
| | GGCTTCGTCAAACTGATGGAATTCTCCAAGAAATCCGAAGAGGACGAAAAGAATGAAGAAG |
| | AAGAAGAGGAAGCTGACGTCTTGAAATTGCAAAAGTCTCAATCTATTGCCGCCTCTCAAAAC |
| | AATGATGCTGTTGCTGGTATTTTGGTTGGTGAGGAAGAAAAAGCTAAGAACGGTATTTCCTT |
| | CTCCGTTTACACCAACTACTTGAAAGAAGGTGGCGGTATTTTTGGTAAATTTGCTGGTCCATT |
| | GACCTTGTTGTTTTTGACTTTCGATGTGTTCACCTCCATTTTCATCAACGTTTGGTTGACTTTCT |
| | GGATAACCGATAAGTGGTCTTATAGATCCCAAGGTTTCTACATCGGTATCTACGTTATGCTGG |
| | TTTTGATGAACATCGTCGTTATTGCTTGCTGCTTGATTTTGATGGGTTACATTTCTACTACCTC |
| | CGCCAAGTCTTTGAATTTGAAAGCTATGAAGAGGATCCTGCACTCTCCAATGTCTTTTATGGA |
| | TGTTACTCCAATGGGCAGAATCTTGAACAGATTCACTAAGGATACTGACGTGTTGGATAACG |
| | AAATCGGTGAACAAGTTAGAATGTTCTTGCATCCAGCAGGTTTCGTTGTTGGTGTTGTTATTT |
| | TGTGCATCATCTACTTGCCATGGTTCGCTTTGGTTATTCCACCTGTTGTTATCGTTTCAACTTT |
| | GACCGCTAGTTTCTACCAAGCTTCTTCTAGAGAAATCAAGAGATTGGAAGCCGTTCAAAGGT |
| | CTTTCGTCTACAACAATTTCAACGAGATCTTGAACGGTATGACTACCTTGAAAGCTTACAGAT |
| | CTACCTCTAGATTCTTGGCCAAAAACGAAGTTTTCGTCGACTCTATGAATGAGGCTTACTTCA |
| | TCGTTATCGCTAACCAGAGATGGATTTCCATTCACATGGATTTGGTTGCTGTTTGCTTGTTGG |
| | CAGTTGTTTCTTTATTGACTGTTACCAGGCAGTTCAACATTTCTGCTGCTTCTACCGGTTTGGT |
| | TGTTACTAATGTTTTACAGATCGGTGGCCTGATGTCATTGATTATGAGAGCTTATACCACCGT |
| | CGAAAACGAAATGAATTCTGTCGAAAGGTTGTACCAATACGCCAACACTTTGGTTCAAGAAA |
| | AGCCATACAGAGTGAACGAAACTGTTCCAGCTCCATCATGGCCAGAAACTGGTTCTATTAAG |
| | TTCGAAAATGTCTCCCTGAGATACAGAGAAGGTTTGCCTATGGTTCTGAAGAACTTGTCTAT |
| | GTCTGTTAAGGGCTCTGAAAAGATTGGTATTTGTGGTAGAACTGGTGCCGGTAAATCTTCTA |
| | TTATGACTGCCTTGTACAGGTTGTCTGAATTAGCTGAAGGTGCCATCTTGATTGATGATGTTG |
| | ACATCTCTAAACTGGGCATGTTCGAATTGAGATCCAAGTTGTCTATTATCCCACAAGATCCAG |
| | TTTTGTTCCAAGGCTCCATTAGAAAGAATTTGGATCCATTCGGTGAATCCGATGATGAACACT |
| | TATGGGATGCTTTGAGAAGATCTGGTTTGACCGATGCTTCTATCTTGACTACTATTAAGGCCC |
| | AAACTAAGGATGATCCAAACTTCCATAAGTTCCACTTGGATCAAATCGTCGAAGATGAAGGT |
| | TCTAACTTCTCATTGGGTGAAAGACAACTATTGGCTTTAGCAAGAGCCTTGGTCAGAAATTCC |
| | AGAATTTTGATTTTGGACGAAGCCACTTCCTCCGTTGATTACGAAACTGATGCTAAGATCCAA |
| | AACACCATTACCAACGAGTTTTCCAACTGTACCATTTTGTGTATCGCCCACAGACTGAAAACC |
| | ATCTTGAACTATGATAGGATCCTGGTGTTGGAACAAGGTGAAATTGAAGAATTCGATACCCC |
| | AATCAGGTTGTACGAAAATGATGGTATCTTCAAGCAGATGTGCGAAAGATCTGATATCACCA |
| | GAGAAGATTTTCACATCCAGAAGTAA |
| SEQ ID | MAEDLGLSSPKTDPEDSSKLVLERRIMTPFLSKKVPPVPTEAERKYFPKNKNPLSLIFYWWLNPIM |
| NO: 930 | KVGYKRTLTPNDLYKLTPEMKIDHTYDKFEKILMKIVEKDRAKALAEDPSLTEEDLIRRPYPKYALPK |
| | ALFLTFKWKYILALFFKVLADVCGVCNPLLSKKLIAFVERKTSDPSLAVNDGIGYALGCTFLVLFSGI |
| | MINQSLLHSLTTGAHCKGILTTALLRKSFKADAETRHKYTSGRITSIMSTDLARIDLAIGFQPFGLTF |
| | PIPVIIAIALLIVNIGVSSLAGIAVFIISIVAIGGSARFLMKMRRGANKYTDKRISLMREILQSMKMIK |
| | YYSWEDAYEKSVIQQRTKEVGIILKMQSIRNGLLAFSIALPAFTSMIAFLVLYGVSSNKNPANIFPS |
| | VSLFGTLAQQTMMLPMALATGADAMIGLGRVREFLESGVDLKDPEEFDGHAPETDEEVKELPS |
| | DVAIEAKDATFIWEKFAEVDSEETSDASKSDKETSSTLSEGELKKTLSSEEEEGNEQYTNSVFEGFH |
| | NINLEIKKNEFIIVTGAIGSGKSSLLTALAGFMKRTTGSLSVGGSVLLCGTPWVQNATVRENITFGL |
| | EYDEERYERVLDACALRDDLKLFTGGDLTEIGERGITLSGGQKARINLARAVYADKEIVLFDDVLSA |
| | VDARVGKHIVDDCLCDFMGHKTRILATHQLSLIDKADRVIFLNGDGTIHIGTVNELLQSNEGFVKL |
| | MEFSKKSEEDEKNEEEEEEADVLKLQKSQSIAASQNNDAVAGILVGEEEKAKNGISFSVYTNYLKE |
| | GGGIFGKFAGPLTLLFLTFDVFTSIFINVWLTFWITDKWSYRSQGFYIGIYVMLVLMNIVVIACCLIL |
| | MGYISTTSAKSLNLKAMKRILHSPMSFMDVTPMGRILNRFTKDTDVLDNEIGEQVRMFLHPAGF |
| | VVGVVILCIIYLPWFALVIPPVVIVSTLTASFYQASSREIKRLEAVQRSFVYNNFNEILNGMTTLKAY |
| | RSTSRFLAKNEVFVDSMNEAYFIVIANQRWISIHMDLVAVCLLAVVSLLTVTRQFNISAASTGLVV |
| | TNVLQIGGLMSLIMRAYTTVENEMNSVERLYQYANTLVQEKPYRVNETVPAPSWPETGSIKFEN |
| | VSLRYREGLPMVLKNLSMSVKGSEKIGICGRTGAGKSSIMTALYRLSELAEGAILIDDVDISKLGMF |
| | ELRSKLSIIPQDPVLFQGSIRKNLDPFGESDDEHLWDALRRSGLTDASILTTIKAQTKDDPNFHKFH |
| | LDQIVEDEGSNFSLGERQLLALARALVRNSRILILDEATSSVDYETDAKIQNTITNEFSNCTILCIAHR |
| | LKTILNYDRILVLEQGELEEFDTPIRLYENDGIFKQMCERSDITREDFHIQK |
| SEQ ID | ATGTCCGTTGAAGAAACCGCTGGTACTAATTTGGCTCTACAAAAGAGATCTTTGACCTTCTTG |
| NO: 931 | TTCGGTAAAAAGGTTCCACCATTGCCATTGGAAGAAGAGAGAAAAGTTTTTCCACATTACCA |
| | CACCAACATCATCTACAGAGCTTTCTTTTGGTACTTGACCCCATTGATGAGAGTTGGTTACAA |
| | AAGAACATTGCAACCAGAGGACATGTACTTGTTGGATGAAGAACAAACCATCGACTACATGT |
| | ACAAGAAGTTCATTGCCTCCGTTGATTCCGATTTGGAAAAGCAAAAGGCTAAGCACATTCTG |
| | AAAAAGTGCAAAGAAAGAGGTGAAACCCCAGAATCTTCTTCAGTTGATCCAGAAACTGACTT |
| | GGAAGATTTCGAATTGCACTACGTTTACTTGGTCAAGGGTTTGATTAGAGTTTTCGGTTGGC |
| | AATATGGTTGGGCTACTTTCATTAAGGCTTTCGCTGATTTGTCATCTGCTTTGTTGCCTTTGGT |
| | TTTGAAGCGTTTGATCAACTTCGTTGAAAGAAAGGCTTATGGTTTGGAACCACATGTTGGTA |
| | AAGGTATTGGTTACGCTTTTGGTGTTTCCTTGATGGTTTACTTTTCTGGTTTTGCCTTCAACCA |
| | CTTCTTCTACAACTCTACTACTGTTGGTGCTAAGGTTAAGGCTGTTTTGACAAAAGCTTTGTTG |
| | GAGAAGTCTTTCACCTTGGATGCTAGAGGTAAACATAAGTTCCCAATCGGTAAGATCAACTC |
| | CATTATGGGTACTGATTTGACCAGAGTTGATTTGGCTTTGGGTTTCTTTCCATTCTTGTTGGGT |
| | TTTCCCATTCCATTGATCGTTATCATCGTTATGTTGTTGGTTAACATCGGTGTTTCTGCTTTGG |
| | CTGGTATTGGTGTTTTTGTGATGTCTATTTTCTTGACCGGTTTCATCGTTAGAGAGTTGTTCAG |
| | ATTGAGAGTTATCGCTAACGTTTTCACCGACGAAAGAGTCAATTTGGTCAAAGAGCTGTTGA |
| | AGAACTTCAAGATGATCAAGATGTACGGTTGGGAGAACTCCTACTTCAAAGAATTCATGGAC |
| | ATCAGGCAGAAAGAAATGACTACCGTTTTGAGAATGCAAGTTGCCAGAAACGTTTTGATTGC |
| | TGTTGCTATTTGGCTGCCAATCGTTTCTTCTATGGTTGCATTTTTGGTCTTGCACAAGATCGAT |
| | TCTAACAGAACAGTTGGTGACATCTTCTCCTCATTGTCTTTGTTCCAAGAATTGACCACTCAGT |
| | TTTTGATGGTTCCAGCTGCTTTAGCTATGTCTACCGATATGGTTATTGCTTTCAAGAGGATCTC |
| | TCAGTTGTTGTCTTGTCCAGATGGTCAAGAATTGGCTACCTTTTTCGATACTTTGGATGATCCT |
| | AAATTGGCCCTGCAATTGAAGAATGCTTCTTTTCAATGGTTCACCTTCGAAGATGAGAAGCCA |
| | GAAGATTCCAAATCTGAAGATGAACCAGAATCCTCCGATCAGAAATCTGAAAAATCTGCTGG |
| | TTCCGAAGTTGTCAAGACTGAATTTCCAGGTTTGCTGAACTTGAACTTGTCTATTGCTAAGGG |
| | TGAATTCGTTGTTGTTACCGGTTCTATTGGTTCCGGTAAATCTTCTTTGTTGAACGCTTTTTCC |
| | GGCTTTATGCCAAAAACTTCTGGTTCTGTTGCTAAGAACGGTTCTTTGATGTTATGTGGTTAT |
| | CCATGGGTTCAAAACGCTACCATCAAAGAAAACATCGTGTTCGGTGAAGAATACGACCAAG |
| | AAAAGTACGATACCATCGTTAAGGTCTGTTCTTTGACTGGTGATTTCGAACAATTTTCCGCTG |
| | CTGATAAGACTGAAGTTGGTGAAAGAGGTATTACATTGTCTGGTGGTCAAAAGGCCAGAAT |
| | CAATTTGGCTAGAGCAGTTTATTCCGACAAGGACATCATCTTGTTAGATGACGTTTTGTCAGC |
| | TGTTGATGCCAAGGTTGGTAAATCTATTATGAAGGATTGCATCATGGGCTACCTGAAAAACA |
| | AGACTAGAGTTTTGGCTACCCATCAGTTGTCCTTGATTGATTCAGCAGATAAGATCATGTTCT |
| | TGAACGGTGATGGTACTGTTGATTACGGTACTTTGGACGAAGTTAAGTCTAGAAACCCAGAA |
| | TTCGTCAGGTTGATGGAATTCTCTCATAACGTTGATGATGATGACGAGGACGAAAATGAACC |
| | TGAACAAGAAAAGAATGATGATTTCGGTGCTGATGACGATGAAGATGGTAGATTGATCAGA |
| | GCTGAAGAAAAGGCTGTTAACGCTATTTCTTGGGATGTTTACAGAACCTACATCAAAGCTGG |
| | TTCTGGTAAGTTGGGTTACTTTTACCCAGTTGTAGTTGTTTTGGCCTTCTCTGTTTCTACCTTCT |
| | GTTTGTTGTTTGTCAACAACTGGTTGTCCTTTTGGGAATCCGGTAAGTTTCATGAACCAGGTT |
| | CTTTTTACGAGGGCATCTATATTATGTTCGGCTTCTTGTGCCTGATCTTCTTGATCGTTGAATT |
| | CTTGTTCTTGGTCTACTTCTGTAACATGGCTGCTAGAAGATTCAACATCTTGGCTTTTAAGAG |
| | GTTGTTGCATACCCCAATGTCTTTCTTGGATACAACTCCAATGGGTAGAGTCTTGAACAGATT |
| | CACTAAGGATACTGATGCTATGGACAACGAAATCCAAGACCAATTCAGACAATTCTTCCAAC |
| | CATTGGCTACTATCGTTGGTACTTTGATCTTGTGTATCATCTACTTGCCATGGTTCGCTATTGC |
| | TATTCCAATTGTCTACGCTCTGTTCTACTTGATCTCTAACTTTTACTTGGCCTCCTCCAGAGAA |
| | ATCAAAAGATTGGAAGCTTTGAAGAGGTCCTTCGTTTACTCCCATTTTAACGAATCTTTGTCC |
| | GGTATGGACACTATTAAGGCTCATAACTCTGAAACCAGATTCTTGAACACTAACGCCCACTTG |
| | ATTAACGACATGAATGAATCTTACTACACCTTCATTGCCGTCCAAAGATGGTTGGCTTCTAAC |
| | TTGGAATTATTGGCTTCTTTGGTCTGCTTGCTGATTTGCTTGTTGTGCGTTTTTAGGGTGTTCA |
| | ACATTTCTGGTGCTTACACCGGTTTGGTGTTGACTTACGTTGTTAATATTGTCGGCTTGGTGT |
| | CCTTCATGTTGAGATCTATGACCGAAGTCGAAAACCAGATGAACTCTGTTGAAAGGTTGAAG |
| | TTTTACGCCATCGACTTGCAACAAGAAGCTGCTTACGATATTCCAGAAAGAGATCCTGAACCT |
| | ACTTGGCCAGCTTCTGGTAGTATTTCATTTCAAGATGTCTCCATGAGATACCGTGAAGGTTTG |
| | CCATATGCTGTTAAGGGTTTGTCTTTGGATGTTGCTGGTTCAGAAAAGATCGGTATTTGTGGT |
| | AGAACTGGTGCTGGTAAATCCTCTGTTATGTACTCCTTGTTTAGATTGGCTGAATTCGAGGGT |
| | AAGATTACCATTGATGGTGTCGATATTTCCCAAATCGGTCTGCATAAGTTGAGGACCAAGAT |
| | TTCCATTATTCCACAAGATCCAGTGTTGTTCTCTGGTAATGTCAGATCTAATTTGGACCCATTC |
| | AACGACAGAACCGATGAAGAATTATGGTCTGCATTGGAAAAAGCCGGTTTGATCGATGGTT |
| | CTATTTTGGAACAAGTCAAAAAGCAGCAAAAGACCGATGCTAACTTGCACAAATTCCACTTG |
| | GATAGAGTCGTTGAAGATGACGGTTCTAATTTCTCATTGGGAGAAAGACAATTGCTGGCTTT |
| | GGCAAGAGCTTTGGTTAGAGGTGCTAGAATATTGGTTTTAGATGAAGCCACCTCTTCCGTTG |
| | ACTACGAAACAGATGCTAAAGTTCAAAAGACCATTACCGAAGAATTCGCTCAATGTACTGTT |
| | TTGTGCATTGCCCATAGATTGAAAACCATCGTCAAGTACGACAGGATCTTGGTTTTGGACAA |
| | AGGTGAAATTGCCGAATTGGATACTCCAAGAAACTTGTACGAACAGAACGGTATTTTCAGAT |
| | CCATGTGTGATAAGTCCGGTATCGTGGAAGATGATTTCTGA |
| SEQ ID | MSVEETAGTNLALQKRSLTFLFGKKVPPLPLEEERKVFPHYHTNIIYRAFFWYLTPLMRVGYKRTL |
| NO: 932 | QPEDMYLLDEEQTIDYMYKKFIASVDSDLEKQKAKHILKKCKERGETPESSSVDPETDLEDFELHY |
| | VYLVKGLIRVFGWQYGWATFIKAFADLSSALLPLVLKRLINFVERKAYGLEPHVGKGIGYAFGVSL |
| | MVYFSGFAFNHFFYNSTTVGAKVKAVLTKALLEKSFTLDARGKHKFPIGKINSIMGTDLTRVDLAL |
| | GFFPFLLGFPIPLIVIIVMLLVNIGVSALAGIGVFVMSIFLTGFIVRELFRLRVIANVFTDERVNLVKEL |
| | ŁKNFKMIKMYGWENSYFKEFMDIRQKEMTTVLRMQVARNVLIAVAIWLPIVSSMVAFLVLHKID |
| | SNRTVGDIFSSLSLFQELTTQFLMVPAALAMSTDMVIAFKRISQLLSCPDGQELATFFDTLDDPKL |
| | ALQLKNASFQWFTFEDEKPEDSKSEDEPESSDQKSEKSAGSEVVKTEFPGLLNLNLSIAKGEFVVV |
| | TGSIGSGKSSLLNAFSGFMPKTSGSVAKNGSLMLCGYPWVQNATIKENIVFGEEYDQEKYDTIVK |
| | VCSLTGDFEQFSAADKTEVGERGITLSGGQKARINLARAVYSDKDIILLDDVLSAVDAKVGKSIMK |
| | DCIMGYLKNKTRVLATHQLSLIDSADKIMFLNGDGTVDYGTLDEVKSRNPEFVRLMEFSHNVDD |
| | DDEDENEPEQEKNDDFGADDDEDGRLIRAEEKAVNAISWDVYRTYIKAGSGKLGYFYPVVVVLA |
| | FSVSTFCLLFVNNWLSFWESGKFHEPGSFYEGIYIMFGFLCLIFLIVEFLFLVYFCNMAARRFNILAF |
| | KRLLHTPMSFLDTTPMGRVLNRFTKDTDAMDNEIQDQFRQFFQPLATIVGTLILCIIYLPWFAIAI |
| | PIVYALFYLISNFYLASSREIKRLEALKRSFVYSHFNESLSGMDTIKAHNSETRFLNTNAHLINDMNE |
| | SYYTFIAVQRWLASNLELLASLVCLLICLLCVFRVFNISGAYTGLVLTYVVNIVGLVSFMLRSMTEVE |
| | NQMNSVERLKFYAIDLQQEAAYDIPERDPEPTWPASGSISFQDVSMRYREGLPYAVKGLSLDVA |
| | GSEKIGICGRTGAGKSSVMYSLFRLAEFEGKITIDGVDISQIGLHKLRTKISIIPQDPVLFSGNVRSNL |
| | DPFNDRTDEELWSALEKAGLIDGSILEQVKKQQKTDANLHKFHLDRVVEDDGSNFSLGERQLLAL |
| | ARALVRGARILVLDEATSSVDYETDAKVQKTITEEFAQCTVLCIAHRLKTIVKYDRILVLDKGEIAEL |
| | DTPRNLYEQNGIFRSMCDKSGIVEDDF |
| SEQ ID | ATGACCTCTCCAGGTTCTGAAAAGTGTACTCCAAGATCTGATGAAGATTTGGAAAGATCCGA |
| NO: 933 | ACCACAATTGCAAAGAAGATTATTGACCCCATTCCTGTTGTCTAAAAAGGTTCCACCAATTCC |
| | AAAAGAGGACGAGAGAAAACCATATCCATACCTGAAAACTAACCCCTTGTCTCAAATTTTGT |
| | TCTGGTGGTTGAATCCCTTGTTGAGAGTTGGTTACAAGAGAACTTTGGACCCAAACGATTTCT |
| | ACTACTTGGAACACTCCCAAGATATTGAAACCACCTACTCTAACTACGAAATGCACTTGGCTA |
| | GAATCTTGGAAAAGGATAGAGCTAAAGCTAGAGCTAAGGATCCAACTTTGACAGACGAAGA |
| | TTTGAAGAACAGAGAGTATCCAAAGAACGCTGTTATTAAGGCTTTGTTCTTGACCTTCAAGT |
| | GGAAGTATTTGTGGTCCATCTTCCTGAAGTTGTTGTCCGATATAGTTTTGGTCTTGAACCCCTT |
| | ATTGTCCAAGGCTTTGATTAACTTCGTTGACGAGAAGATGTACAACCCAGATATGTCTGTTG |
| | GTAGAGGTGTTGGTTATGCTATTGGTGTTACTTTCATGTTGGGTACATCCGGTATTTTGATCA |
| | ACCACTTCTTGTACTTGTCTTTGACTGTTGGTGCTCATTGCAAAGCTGTTTTGACTACTGCTAT |
| | CATGAACAAGTCTTTCAGAGCTTCCGCTAAATCTAAACACGAATATCCTTCAGGTAGAGTCAC |
| | CTCTTTGATGTCTACTGATTTGGCCAGAATTGATTTGGCTATTGGTTTTCAACCATTCGCCATT |
| | ACTGTTCCAGTTCCAATTGGTGTTGCTATTGCTTTGTTGATCGTTAACATTGGTGTTTCTGCTT |
| | TGGCTGGTATTGCTGTTTTCTTGGTCTGTATCGTTGTTATCTCTGCCTCTTCTAAGTCCTTGTTG |
| | AAAATGAGAAAAGGTGCTAACCAGTACACCGATGCTAGAATTTCTTACATGAGAGAAATCCT |
| | GCAGAACATGAGGATCATCAAGTTTTACTCTTGGGAAGATGCCTACGAAAAGTCTGTTGTTA |
| | CTGAAAGAAACTCCGAGATGTCCATCATCTTGAAGATGCAATCTATCCGTAACTTCTTGTTGG |
| | CCTTGTCTTTATCTTTGCCAGCCATTATTTCTATGGTTGCCTTCTTAGTCTTGTACGGTGTTTCT |
| | AATGATAAGAACCCAGGCAACATCTTCAGCTCCATTTCTTTGTTTTCAGTCTTGGCTCAACAG |
| | ACCATGATGTTGCCAATGGCTTTGGCTACTGGTGCTGATGCTAAAATTGGTCTAGAAAGATT |
| | GAGACAGTACTTGCAATCCGGTGACATCGAAAAAGAATACGAAGATCACGAAAAGCCAGGT |
| | GATAGAGATGTTGTTTTGCCAGATAATGTTGCCGTCGAATTGAACAACGCTTCTTTCATTTGG |
| | GAAAAGTTCGATGATGCCGATGATAACGATGGTAACTCTGAAAAGACCAAAGAAGTTGTCG |
| | TTACCTCCAAGTCATCATTGACTGATTCTTCCCATATCGATAAGTCTACCGATTCTGCTGATGG |
| | TGAGTACATTAAGTCTGTTTTCGAAGGCTTCAACAACATCAACTTGACCATTAAGAAGGGTG |
| | AGTTCGTTATTATCACTGGTCCTATTGGTTCCGGTAAGTCATCTTTGTTGGTTGCTTTAGCTGG |
| | CTTTATGAAGAAAACCTCTGGTACTTTGGGTGTCAATGGTACTATGTTGTTGTGTGGTCAACC |
| | ATGGGTTCAAAACTGTACTGTTAGAGACAACATCTTGTTCGGTTTGGAATACGATGAAGCCA |
| | GATACGATAGAGTTGTAGAAGTTTGTGCTTTGGGTGACGACTTGAAAATGTTTACTGCTGGT |
| | GATCAAACCGAAATCGGTGAAAGAGGTATTACTTTATCTGGTGGTCAAAAGGCCAGAATCA |
| | ACTTGGCAAGAGCTGTTTATGCTAACAAGGACATTATCTTGTTGGACGATGTTTTGTCTGCTG |
| | TTGATGCTAGAGTTGGTAAATTGATCGTTGATGATTGCCTGACTTCTTTCTTGGGTGACAAAA |
| | CTAGAATTTTGGCTACCCACCAACTGTCCTTGATTGAAGCTGCTGATAGAGTTATCTACTTGA |
| | ACGGTGATGGTACAATCCATATCGGTACTGTCCAAGAACTGTTGGAATCTAATGAAGGCTTC |
| | TTGAAGCTGATGGAATTCTCCAGAAAATCCGAATCCGAAGATGAAGAAGATGTCGAAGCTG |
| | CTAACGAAAAGGATGTCTCTTTACAAAAGGCCGTTTCTGTTGTGCAAGAACAAGACGCTCAT |
| | GCTGGTGTTTTGATTGGTCAAGAGGAAAGAGCAGTTAACGGTATTGAATGGGACATATACA |
| | AAGAGTACCTACACGAAGGTAGAGGTAAGTTGGGTATTTTTGCTATTCCAACCATCATCATG |
| | TTGTTGGTCTTGGATGTTTTCACGTCCATTTTCGTTAACGTCTGGTTGTCTTTTTGGATCTCCC |
| | ATAAGTTTAAGGCTAGATCCGATGGTTTTTACATCGGCTTGTACGTTATGTTCGTTATCTTGTC |
| | CGTTATTTGGATTACCGCCGAATTTGTTGTTATGGGCTACTTTTCATCTACCGCTGCTAGAAG |
| | ATTGAACTTGAAAGCTATGAAGAGAGTCTTGCATACCCCAATGCATTTCTTAGATGTTACTCC |
| | AATGGGCAGAATCTTGAACAGATTCACTAAGGATACTGACGTCTTGGACAACGAAATTGGTG |
| | AACAAGCCAGAATGTTTTTACACCCAGCTGCTTATGTTATCGGTGTCTTGATTTTGTGCATCAT |
| | CTACATTCCATGGTTCGCTATTGCAATTCCACCATTGGCTATTTTGTTCACCTTCATCACCAACT |
| | TCTATATCGCCTCTTCAAGAGAAGTCAAGAGAATTGAAGCCATCCAAAGGTCTTTGGTGTAC |
| | AACAATTTTAACGAAGTCTTGAACGGCTTGCAAACCTTGAAGGCTTATAATGCTACTTCCCGT |
| | TTCATGGAAAAGAACAAGCGTTTGTTGAACAGAATGAACGAAGCCTACTTGTTGGTCATTGC |
| | TAATCAAAGATGGATCTCCGTTAACCTGGATTTGGTTTCTTGTTGCTTCGTGTTCTTGATCTCC |
| | ATGTTGTCCGTTTTTAGGGTGTTCGATATCAATGCTTCTTCCGTTGGTTTGGTTGTTACTTCCG |
| | TTTTACAAATCGGTGGCCTGATGTCATTGATTATGAGAGCTTATACCACCGTCGAAAACGAA |
| | ATGAACTCTGTTGAAAGATTGTGCCATTACGCCAACAAGTTGGAACAAGAGGCTCCTTACAT |
| | TATGAACGAAACTAAGCCAAGACCTACATGGCCAGAACATGGTGCTATTGAGTTTAAACATG |
| | CCTCCATGAGATACAGAGAAGGTTTGCCATTGGTTTTGAAGGATTTGACCATTTCAGTAAAA |
| | GGTGGTGAGAAGATTGGTATTTGCGGTAGAACAGGTGCTGGTAAGTCTACTATTATGAATG |
| | CCTTGTACAGGTTGACCGAATTGGCTGAAGGTTCTATTACTATCGATGGTGTCGAAATTTCTC |
| | AGTTGGGCTTGTATGATTTGAGATCCAAGTTGGCCATTATTCCACAAGATCCAGTTTTGTTCA |
| | GAGGCACCATTAGAAAGAATTTGGATCCATTTGGTCAGAACGATGACGAAACTTTGTGGGAT |
| | GCTTTAAGAAGATCCGGTTTAGTTGAGGGTTCTATCTTGAATACCATCAAGTCCCAATCTAAG |
| | GATGATCCAAACTTCCATAAGTTCCACTTGGATCAAACTGTTGAAGATGAGGGTGCTAACTTT |
| | TCATTGGGTGAAAGACAATTGATTGCATTGGCCAGAGCTTTGGTTAGAAACTCTAAGATCTT |
| | GATCTTGGACGAAGCTACCTCTTCTGTTGATTACGAAACCGATTCCAAAATCCAAAAGACCAT |
| | CTCTACTGAATTCTCCCATTGCACCATTTTGTGTATTGCCCATCGTCTGAAAACCATCTTGACC |
| | TATGATAGGATCTTGGTTTTGGAAAAAGGTGAGGTTGAAGAATTCGATACACCAAGGGTCTT |
| | GTACTCTAAGAATGGTGTCTTTAGACAGATGTGCGAAAGATCTGAAATTACCTCTGCTGATTT |
| | CGTCTAA |
| SEQ ID | MTSPGSEKCTPRSDEDLERSEPQLQRRLLTPFLLSKKVPPIPKEDERKPYPYLKTNPLSQILFWWLN |
| NO: 934 | PLLRVGYKRTLDPNDFYYLEHSQDIETTYSNYEMHLARILEKDRAKARAKDPTLTDEDLKNREYPK |
| | NAVIKALFLTFKWKYLWSIFLKLLSDIVLVLNPLLSKALINFVDEKMYNPDMSVGRGVGYAIGVTF |
| | MLGTSGILINHFLYLSLTVGAHCKAVLTTAIMNKSFRASAKSKHEYPSGRVTSLMSTDLARIDLAIG |
| | FQPFAITVPVPIGVAIALLIVNIGVSALAGIAVFLVCIVVISASSKSLLKMRKGANQYTDARISYMREI |
| | LQNMRIIKFYSWEDAYEKSVVTERNSEMSIILKMQSIRNFLLALSLSLPAIISMVAFLVLYGVSNDK |
| | NPGNIFSSISLESVLAQQTMMLPMALATGADAKIGLERLRQYLQSGDIEKEYEDHEKPGDRDVVL |
| | PDNVAVELNNASFIWEKFDDADDNDGNSEKTKEVVVTSKSSLTDSSHIDKSTDSADGEYIKSVFE |
| | GFNNINLTIKKGEFVIITGPIGSGKSSLLVALAGFMKKTSGTLGVNGTMLLCGQPWVQNCTVRDN |
| | ILFGLEYDEARYDRVVEVCALGDDLKMFTAGDQTEIGERGITLSGGQKARINLARAVYANKDIILL |
| | DDVLSAVDARVGKLIVDDCLTSFLGDKTRILATHQLSLIEAADRVIYLNGDGTIHIGTVQELLESNE |
| | GFLKLMEFSRKSESEDEEDVEAANEKDVSLQKAVSVVQEQDAHAGVLIGQEERAVNGIEWDIYK |
| | EYLHEGRGKLGIFAIPTIIMLLVLDVFTSIFVNVWLSFWISHKFKARSDGFYIGLYVMFVILSVIWITA |
| | EFVVMGYFSSTAARRLNLKAMKRVLHTPMHFLDVTPMGRILNRFTKDTDVLDNEIGEQARMFL |
| | HPAAYVIGVLILCIIYIPWFAIAIPPLAILFTFITNFYIASSREVKRIEAIQRSLVYNNFNEVLNGLQTLK |
| | AYNATSRFMEKNKRLLNRMNEAYLLVIANQRWISVNLDLVSCCFVFLISMLSVFRVFDINASSVG |
| | LVVTSVLQIGGLMSLIMRAYTTVENEMNSVERLCHYANKLEQEAPYIMNETKPRPTWPEHGAIE |
| | FKHASMRYREGLPLVLKDLTISVKGGEKIGICGRTGAGKSTIMNALYRLTELAEGSITIDGVEISQLG |
| | LYDLRSKLAIIPQDPVLFRGTIRKNLDPFGQNDDETLWDALRRSGLVEGSILNTIKSQSKDDPNFHK |
| | FHLDQTVEDEGANFSLGERQLIALARALVRNSKILILDEATSSVDYETDSKIQKTISTEFSHCTILCIA |
| | HRLKTILTYDRILVLEKGEVEEFDTPRVLYSKNGVFRQMCERSEITSADFV |
| SEQ ID | ATGCCCACAATAAGACAAGAGCTGCGTCACTCTTCCAGTGGAAGTGAGAATGAGAAAGCCG |
| NO: 935 | AGTCGCTATATGTCAAGAACGAAGGAAAGCTTGACAAAGTTGCAACGCAGAACAGTTATTAT |
| | GAGGTGGACCGTAATAGACCGGAGACTTTTATGAATAGTGACGACTTAGAGAAAGTGACGG |
| | AGTCAGAGATATATCCCCAGAAACGTATGTTTAGCTTTCTACATAGCAAGAAGATACCACCG |
| | ATCCCTACTGACGAGGAGCGTCCTGTGTATCCCCTATTCCACGCAAATTGGATATCAAGGATA |
| | TTCTTCTGGTGGGTCTTTCCCATCTTAAGGGTCGGATATAAAAGGACTCTTCAGCCTGGAGAC |
| | TTATGGAAAATGGACGACAGGATGAGTATTGAGACTCTATATGCCGACTTTGAGAGGTATCT |
| | GGAAGTGTATAGGGAGAAAGCCAGGGTTCAATATAGGAAGGAGCACCCTAACGCGACAGA |
| | AGAGGAAATAATAGAGAATGCTGTCATGCCTAAGCATACTCTGGTCAAAGTGCTTTTATATA |
| | CATTTAAATGGCAGTATTTCTTAGCCTTCGCCGCGATGGCATTATCCAACGCAGCATCAGCCT |
| | TCTTACCGATGGTTACAAAGAGGCTTATTGATTTTGTCAGTGAGAAAAGCTTCTATCCTGGGT |
| | TAAAAGTGAACGCAGGAGTAGGCTACGCCATCGGGTCATGTGTCATGATGTTACTAAATGG |
| | AGTCCTGTTCAATCATTTCTTTCACAATAGTCAGCTTACGGGCGTCCAGGCTAAATCTGTATT |
| | AATCAAAGCCATACTTACTAAATCTATGAAACTTTCAGGCTTTAGTAGGCACCGTTTTCCTAG |
| | CGGCAAAATAACTTCCATCATGAGTACGGATTTATCTAGGTTAGAACTAGCAATAATTTTCCA |
| | GCCGTTACTAGGAGCGTTCTTCGTGGCCGTCGCGATTTGCATAGTTTTATTAATCATAAATCT |
| | TGGCCCGATAGCCCTTGTCGGGGTGGGCATATTCGTCGTAGCAATGTTCTTTTCCGCCTATGC |
| | TTTTAAACGTCTAATCTCTGTGAGGAAGAAGACAAATATATTTACAGACGCTCGTGTCACTAT |
| | GATGCGTGAGATCTTGAATTCGATGAAAATGATCAAATTTTATGCATGGGAAGACGCATACG |
| | AGGCATCAGTGCACGATCAACGTTCGAAGGAGATAAGCAAAACGAGGATTATGCAATTTAC |
| | TAGGAACTTTGTAACCGCATTAGCAGTCTGTTTAACTAATATCTCCAGTATGGTGACCTTTCT |
| | AGCATTGTATAAGGTACGTAATCACGGCCGTACCCCTGCTAATATATTCTCCAGCTTAAGTCT |
| | GTTTCAAGTTCTTAGCATACAGATGTTCTTTCTACCCATGGCGTTAGGCACAGCCGTGGACGG |
| | ATCCATCGCACTTAACCGTTGTCAGGAACTTTTCGAGGCCACCGAGGAAGAGCATGACATAG |
| | ACGTAGACTTTCCCCCTTGCGACGACCCCGACTTAGCACTTAAGGTAGTAAATGGGTCCTTTG |
| | AGTGGCAGGACTTCGAGGCTGAGGAGAATAGGTTAGCGACATTAATGGAGATAGAGGAGA |
| | AGAAGAAGAAGAAAACGAAAAGTAAGAAGGACAAAGCACCCGAGCCAAAGCACGAGGCA |
| | GCATCAATAAAACCCGGCCACCTAAGCGACACAGAGAGAGAGTCATTCAAAGGGTTTCATA |
| | ATTTAAATTTCGAAGTTAAGAAGGGCGAATTAATAATTATAACCGGATCTATCGGCACCGGC |
| | AAGACAAGTCTGTTAAACGCGTTGGCCGGATTTATGCGTAAAACAGAGGGAGACGTATATA |
| | AGAATGGTTCTCTATTACTTTGCGGATACCCGTGGGTACAGAATGCAACAGTCCGTGATAAC |
| | ATTCTGTTTGGATCCCCGTACGACAAAGCTAGGTATAAAGAGGTCATACGTGTGTGCTCGCT |
| | TCAGGCTGACTTAGACATATTACCCGCGAATGACAAAACAGAGATCGGAGAGAGGGGTATT |
| | ACCTTATCAGGCGGACAGAAGGCCAGGATAAATTTAGCGAGGTCTGTATATAAGTCAATGG |
| | ACACATATCTGTTTGACGACGTGCTAAGTGCCGTGGACGCACGTGTCGGCAAACATATCATG |
| | GACGAGTGCATGTTAGGGAGATTAGGAAATAAGACGCGTATCTTAGCCACACATCAATTATC |
| | GTTAATAGACCGTGCAAGCCGTGTGATCTTCTTAGGTACTGACGGATCCTTCGACTTTGGATC |
| | AGTCACGGAACTTAAGAAACGTAATGCAGGTTTCAATAAACTAATGGAGTTTGCTAATAAAT |
| | CATCAGATAAGGAAGAAGGAGAGCTGGACAGTACCGAGGCAAGCGGCGACGACGTTTCAA |
| | CAGCAGAGGAGTTAGAGCATTTTAGGGACGACGACGGCCAGCGTGAGATGGATGCGTCTA |
| | GATTGAAGAAGGAGTTATCAAAACGTTCGTATGAATCTAGTGTGGATGAGAACGAGGCCGC |
| | AGGAAGGCTGATGGCAAAGGAAGAGCGTGCAGTGAATAGCATCGGCTTTGATGTATATAAG |
| | AATTATATATCGGCAGGAGTGGGAAAGAAAGGATTCGTACTGCTTCCATTTTATGTGATTTT |
| | ACTTGCAGTCACGACATTTTCGCTTCTATTCAGTAGTGTTTGGTTATCATTCTGGACTGAAGA |
| | CAAATTCAAGAGGCAGGCTGGCTTCTATATGGGTATGTATATATTCTTCGTATTCTTCAATTA |
| | CTTTTGCACTACCGGCCAGTTTACATTACTTTGCTACCTGGGACTTACAGCGTCGAAAATGTT |
| | AAACCTGAAAGCAGTAAAGAGGATTCTGCACACACCTATGTCATTTATCGACACGACGCCTA |
| | TCGGACGTATTTTAAATAGGTTTACCAAAGACACAGACACATTAGACACTGAGCTGACCGAG |
| | TCGGTCAGACTGTTTGTGTATCAGACAGCAAATATCATTGGGGTAGTGATAATGTGTATTATT |
| | TATCTACCCTGGTTTGCGATAGCAGTCCCGTTTCTTGTAATTATATTTGCACTTGTGGCCAATC |
| | ATTATCAGAGCTCGTCTAGGGAGATTAAAAGATTAGAGGCTATTCAGAGGAGCCACGTGTTT |
| | AATAATTTTAATGAGGTGTTGGGCGGCATCGATACGATAAGGGCATATAGAGGGCAGGAGC |
| | GTTTTCTAATGAAGAATGATTTTCTAACAAATAAAATGAACGAGGCCGGATATTTAGTGGTA |
| | GCTGTCCAGAGGTGGGTCTCCATCGCTCTGGACATGATAGCGATGGCTTTTGCACTTATCATT |
| | GCATTATTGTGCGTCACTAGGCAATTTCACATATCACCTTCCTCGGTGGGAGTGTTGCTAACG |
| | TATGTATTACAGCTGCCCGGATTACTAAATACGCTAATGCGTGCAATGACGCAGGGAGAGAA |
| | TGACATGAACTCGGCTGAGCGTCTGATAGCGTACGCAACAGACTTACCTCTGGAAGCAAACT |
| | ATAGGAAACCTGAGATGACCCCTGCCGAGCCATGGCCTAGCCACGGAGAGATTGTGTTCGA |
| | CGACGTATCATTAGCCTATAGGCCTGGACTGCCTTTGGTACTTAAGAATGTGTCTATTGACAT |
| | AGGATCAGGAGAGAAAATTGGAATATGCGGACGTACTGGCGCAGGAAAATCAACGATCAT |
| | GACCGCTTTATACAGAATTTGTGAGCTTCACTCTGGAACCGTATCAATTGACGGAGTGGATA |
| | TCTCGAAAATTGGACTGTATGACTTACGTTCGAAATTATCCATAATACCTCAGGACCCGGTGT |
| | TATTTAAAGGGAGTATACGTCGTAATCTTGACCCCTTTAATGAGAGAACTGACGAGCAGCTT |
| | TGGGACGCCCTTGTCAGGAGCGGCGCAGTGGAAGCGTCAGAGATAGCCGAGGTCAAAGCC |
| | CAGTCGCCCGAGACATCAGGAGCATATGCCAACATGCACAAATTTCACCTTAGGCAGGAAGT |
| | AGAGGACGATGGATCAAACTTCAGCCTAGGAGAACGTCAGCTACTAGCGTTAACCAGGGCC |
| | CTTGTTAGGCAGTCGAAAATACTTATTCTAGACGAGGCAACCTCGTCGGTCGACTATGAGAC |
| | AGACGCCAAAATTCAGGCGAAAATTGTACAGGAGTTTTCGAGCTGCACAATATTATGTATCG |
| | CTCACCGTCTAAATACTATCCTAGACTATGACAGGATATTGGTCCTGGAGCAAGGCTCAGTA |
| | GCGGAGTTCGACACGCCTAAAGCGTTATTCCGTGCTGGCGGGATATTCACGGAGATGTGCC |
| | AGCGTAGTGGAATAACTTCGGCAGACTTCAAGGAAAATTGA |
| SEQ ID | MPTIRQELRHSSSGSENEKAESLYVKNEGKLDKVATQNSYYEVDRNRPETFMNSDDLEKVTESEI |
| NO: 936 | YPQKRMFSFLHSKKIPPIPTDEERPVYPLFHANWISRIFFWWVFPILRVGYKRTLQPGDLWKMDD |
| | RMSIETLYADFERYLEVYREKARVQYRKEHPNATEEEIIENAVMPKHTLVKVLLYTFKWQYFLAFA |
| | AMALSNAASAFLPMVTKRLIDFVSEKSFYPGLKVNAGVGYAIGSCVMMLLNGVLFNHFFHNSQL |
| | TGVQAKSVLIKAILTKSMKLSGFSRHRFPSGKITSIMSTDLSRLELAIIFQPLLGAFFVAVAICIVLLIIN |
| | LGPIALVGVGIFVVAMFFSAYAFKRLISVRKKTNIFTDARVTMMREILNSMKMIKFYAWEDAYEA |
| | SVHDQRSKEISKTRIMQFTRNFVTALAVCLTNISSMVTFLALYKVRNHGRTPANIFSSLSLFQVLSI |
| | QMFFLPMALGTAVDGSIALNRCQELFEATEEEHDIDVDFPPCDDPDLALKVVNGSFEWQDFEAE |
| | ENRLATLMEIEEKKKKKTKSKKDKAPEPKHEAASIKPGHLSDTERESFKGFHNLNFEVKKGELIIITG |
| | SIGTGKTSLLNALAGFMRKTEGDVYKNGSLLLCGYPWVQNATVRDNILFGSPYDKARYKEVIRVC |
| | SLQADLDILPANDKTEIGERGITLSGGQKARINLARSVYKSMDTYLFDDVLSAVDARVGKHIMDE |
| | CMLGRLGNKTRILATHQLSLIDRASRVIFLGTDGSFDFGSVTELKKRNAGFNKLMEFANKSSDKEE |
| | GELDSTEASGDDVSTAEELEHFRDDDGQREMDASRLKKELSKRSYESSVDENEAAGRLMAKEER |
| | AVNSIGFDVYKNYISAGVGKKGFVLLPFYVILLAVTTFSLLFSSVWLSFWTEDKFKRQAGFYMGM |
| | YIFFVFFNYFCTTGQFTLLCYLGLTASKMLNLKAVKRILHTPMSFIDTTPIGRILNRFTKDTDTLDTEL |
| | TESVRLFVYQTANIIGVVIMCIIYLPWFAIAVPFLVIIFALVANHYQSSSREIKRLEAIQRSHVFNNFN |
| | EVLGGIDTIRAYRGQERFLMKNDFLINKMNEAGYLVVAVQRWVSIALDMIAMAFALIIALLCVTR |
| | QFHISPSSVGVLLTYVLQLPGLLNTLMRAMTQGENDMNSAERLIAYATDLPLEANYRKPEMTPA |
| | EPWPSHGEIVFDDVSLAYRPGLPLVLKNVSIDIGSGEKIGICGRTGAGKSTIMTALYRICELHSGTVS |
| | IDGVDISKIGLYDLRSKLSIIPQDPVLFKGSIRRNLDPFNERTDEQLWDALVRSGAVEASEIAEVKA |
| | QSPETSGAYANMHKFHLRQEVEDDGSNFSLGERQLLALTRALVRQSKILILDEATSSVDYETDAKI |
| | QAKIVQEFSSCTILCIAHRLNTILDYDRILVLEQGSVAEFDTPKALFRAGGIFTEMCQRSGITSADFK |
| | EN |
| SEQ ID | ATGGCCGAATTGGAAAAGGGTGATGAAGCTCAACCAGCTTTACAACATAGATTGTGTACACC |
| NO: 937 | CTTGTTGTCCAAAAAGGTTCCACCAGTTCCAAGAGATGAAGATAGACCAGTTCATCCAAAAG |
| | CTACCAATCCATTCTCTTGGTTTTTCTTCACTTGGTTGACTCCAGTTTTGTTGAGAGGTTACAA |
| | GAGAACTTTGTTGCCAGAGGATATGTTCAAGTTGCATGACGAAATGACCGTTGAACATTTGG |
| | CTGGTAAGTTCCAAGCTATCTTCGATAGAAGATTGGCTGCTGATAAGAAGAAGTACTTGAAG |
| | AGAAGAAGGCAATTGGCCTTGAAGAAAGGTGATTCTGATGTTTCTGCTAGAACCGACGAAG |
| | ATTTGATGTTGGAATACGAACCATCTAAGTCCCTGTGTTTCTTGTCCTTGTACGAAACATTTTT |
| | GTGGCAATACTCCATGGCTTTGTTGTTCGGTATGTTGGGTTTAGTTGGTCAAGCTTGTAATCC |
| | TTTGTTGTCCCGTAAGTTGATTAACTTCGTTGAATTGGAAGCCTTGGGTATCCCAACAAAAAT |
| | TGGTACTGGTATTGGTTACGCTTTCGGTGTTGCTATTTTGATGTTTGTTTCCGACGTCTTGCAC |
| | AATCAAGGTGTTTATTTTGCTATGTTGACCGGTGCTCAAATTAGAGCTATTTTCACAAAAGCC |
| | CTGCTGGACAAGTCTTTCAAATTGAATACCAGATCGAGGAAGAAGTTCCCACCATCTAAAAT |
| | TACCTCCATCATGTCTACCGATGTTTCCAGAGTTGACTTAGGTACAGGTTTTTCTATCTACGGT |
| | TTCGTCTTGATTTTCCCAGTTGGTGTTTCCATTGGTATCCTGATCTACAATATTAAGGCTCCAG |
| | CTATGGTTGGTGTCGGTTTGATGTTAGCTTTTGTTTTTGTTAGCGGTGGCTTGTCTACCTTGTT |
| | GTTCTCTTTTAGAAAAACTGCTCAAAAGGCCACCGATTCTAGAGTTGGTTATATGAAGGAAG |
| | TCCTGAACAACCTGAAGATGATCAAGTTTTACTCTTGGGAAAAGCCATACCATGCCTTGATTA |
| | CTAAGATCAGACGTAGAGAAATGGCCTACTTGTTGAGAATGGAAATTACCCGTATGATCATT |
| | ATTACCTTGGCTGCTTCTTTGGCCTTGGTTTCATCTTTGGTTTCTTTCTTGACGTTGTACGCTAT |
| | TGCTTCTCCATCTTCTAGAAATCCAGCCGAAATTTTCTCCTCCGTTTCTTTGTTTAACTTGTTGG |
| | CCTCTCAATTCTTGGTCTTGCCATTGTCTATTGCTGGTTCTACTGATGCATTTTTGGGTATGAA |
| | TAGAGTTGCTGCTGTTTTAGCTGCTGACGAAATTGATCCAGAAGATGCTGATACCATCTTGTC |
| | TGAAAGAACTCAAGCTTTGTTGGAGGAAAAGAAGTTGGCTATTACTGTTCAAGACGGTGAAT |
| | TTGAATGGGAGTTGTTTGATTTCGACGACGAAAAGTCCGAAGAAAAAGAAGAACATAAGGA |
| | CGAGAGCAAGAAGAAGAAGAAGAAAGAGTCTAAGAAGAAGGTCAAAAAGTCCGCCAAAGA |
| | TGTCTCTGATACTTCTTCAGCTTCCTCTAACGAAAAAGAGAGGAAGTCTTTTAAGCTGCACAA |
| | CGTTAACTTGGACATTAGACAAGGTGCCTTCGTTGTTATTACAGGTTCTATTGGTTCCGGCAA |
| | :GTCATCTTTGTTGCATGCTTTGGATGGTGCCATGAAGAAATTGTCTGGTGATGTTTACGTTAA |
| | CGGCTCTTTGTTGATGTGTGGTACTCCTTGGATTCAATCTGCTTCATTGAGAGAGAACATCTT |
| | GTTCGGTTCTACCTATGACGAGAAGTGGTACAAAGAAGTTATTAGAGCTTGCTCCTTGGAAT |
| | CCGATTTCGATATTTTGCCAGCTGGTGATTTGACCGAAATTGGTGAAAGAGGTATTACTTTGT |
| | CCGGTGGTCAAAAGGCTAGAGTTTGTTTGGCTAGAACTGTTTACGCTAACTCCTCCATTATCT |
| | TGTTGGATGATGTTTTGTCTGCTGTTGATGCCAAGGTTGGTAAACATATTATGTCCGAATGCA |
| | TCATGGGTATCTTGAAGGGTAAAACTAGAGTTTTGGCTACCCACCAATTGTCCTTGATTTCTG |
| | AAGCTGAACACGTCATTTTCTTGAACGGTGATGGTACTATTTCCAGGGGTACTTTTGAAGAAT |
| | TGAAGTCTACCAACTCTGCCTTCAAGGCTTTGATGGAACATAACAGAAAGTCTGAAGAGAAC |
| | GATGAGGATGAATCAGAACCAGCTTCTGAAGTTGAAGCCTCTGAAAAAGAATTGATCAAGA |
| | GACAGTTGACCAAGCAAACTACTACCCAAGTTTCTTCCGATTCCGTTGAAAAAGTAGAATTG |
| | GATGGCAAGTTGTACGACGAAGAAGAGAAATCTGTTAACGCTATTGGTTGGGATGTTTATG |
| | GTCGTTACATTTTGACTGGTGTCCAAGGTTTTAAGTTCAACTGGTTGTTGTTTGTGATCCTGTG |
| | CTTGTGTATTTTGGGCACTTTTATGTCTCTGTTCACCAACAATTGGCTGTCTTTTTGGATCTCC |
| | AGAAAGTTTGATCAACCAGCCGGTTTTTACATTGGTTTTTACGCTGCTTTTACCGGTTTGGCT |
| | GTTATCTTGATGGTTTTCCAGTTCTGCTCCATCATCTTCGTTATGAACAGAGCCTCTAGGATCT |
| | TGAACATTAAGGCTTTGGGTAAGATCTTGCACGTTCCAATGTCTTTCATGGATACAACTCCAA |
| | TGGGTAGAATCTTGAATAGATTCACCAAGGATACCGACACCTTGGATAATGAGATTGGTGAT |
| | AGAGTCGGTATGGTTGTTAACTTCACTTTCGAAATTTGGGGCGTTATCATCATGTGCATTATC |
| | TACATGCCATGGTTTGCTATTGCTGTTCCTTTTATCGTTGCCGTGTTCATTATTATGGCTAACT |
| | TCTATCAAGCCTCCGGTAGAGAAGTTAAGAGATTGGAAGCTGTTCAAAGATCCCACGTTTAC |
| | AACAACTTCAACGAATCTTTAACTGGTATGCCAACCATTAAGGCCTTCAAGTCCATTCAGAGA |
| | TTCTTGAACAAGAACGTTGCCACGATTAACAAAATGGATGAAGCTTACTTCGTTACCGTCGCT |
| | AATCAAAGATGGTTGGATACTTACTTGTCTTTGTTGGCTACTTTGTTCGCCTTGTTGATTGCTT |
| | TATTGTGTGTCTGCAGAGTGTTCGATATTGGTGCTTCAGCTGTTGGTTTGTTAGTTTCTTATGT |
| | CTTGCAGATCTCCGGCTTGATTTCAATGTTGGTTGTCGTTTTCACCCAAGTCGAACAAGACAT |
| | GAATTCTGCTGAAAGAGTTATGGACTACGTCTACAAGATTCCACAAGAAAGACCATACGAAA |
| | TCTCCGAAACTAGACCACCACCAGAATGGCCTCAAAATGGTGAAATTAGATTCATCAACGTC |
| | GACTTCGCTTACAGAGAAGGTTTGCCATTGACTTTGAGAAACATTAACGCCGATATTAAGCC |
| | ACACGAAAAGATTGGTATTTGCGGTAGAACTGGTGCTGGTAAATCTTCAATTATGGTCGCTT |
| | TGTTCAGAATCGCCGAATTGACTTCTGGTTCCATTATGATTGATGGCATCGATGTTTCTACTTT |
| | GGGCTTGCATGAATTGAGGTCCAACTTGTCTATTATCCCACAAGATCCAGTCTTGTTTAAGGG |
| | CACTATCAGATCTAATTTGGACCCATTCGAAACTAAGACCGATGATGAATTATGGGATACTTT |
| | GCGTAGAGCTGATATTATTGATGCTGCCTCTTTGGAACATGTTAAGACTCAACGTGTTGGTG |
| | ATGATGATTTCCATAAGTTCCACTTGGATAACGAAGTTGACGATGAAGGTGAAAACTTCTCTT |
| | TGGGCGAAAAACAATTGGTTGCATTTGCTAGAGCTTTGGTTAGAGATACCAAGATCATTGTC |
| | TTAGATGAAGCCACCTCTTCTGTTGATTATGCCACTGATTCTAAATTGCAGAAGGCCATCGTT |
| | AGGGAATTCTCTGATAGAACCATTTTGTGCATTGCCCACAGGTTGAAAACTATCTTGCATTAC |
| | GATAGAGTCATCGTCATGGAACAGGGTGAAATCAAAGAATTCGATACTCCATCTCACCTGTA |
| | CAACTCTACTGGTACAATCTTTAGACAAATGTGCGACAAGTCCGGCATCTCTAAAGAAGATTT |
| | TTACGAGTGGTAA |
| SEQ ID | MAELEKGDEAQPALQHRLCTPLLSKKVPPVPRDEDRPVHPKATNPFSWFFFTWLTPVLLRGYKR |
| NO: 938 | TLLPEDMFKLHDEMTVEHLAGKFQAIFDRRLAADKKKYLKRRRQLALKKGDSDVSARTDEDLML |
| | EYEPSKSLCFLSLYETFLWQYSMALLFGMLGLVGQACNPLLSRKLINFVELEALGIPTKIGTGIGYAF |
| | GVAILMFVSDVLHNQGVYFAMLTGAQIRAIFTKALLDKSFKLNTRSRKKFPPSKITSIMSTDVSRV |
| | DLGTGFSIYGFVLIFPVGVSIGILIYNIKAPAMVGVGLMLAFVFVSGGLSTLLFSFRKTAQKATDSRV |
| | GYMKEVLNNLKMIKFYSWEKPYHALITKIRRREMAYLLRMEITRMIIITLAASLALVSSLVSFLTLYA |
| | IASPSSRNPAEIFSSVSLFNLLASQFLVLPLSIAGSTDAFLGMNRVAAVLAADEIDPEDADTILSERT |
| | QALLEEKKLAITVQDGEFEWELFDFDDEKSEEKEEHKDESKKKKKKESKKKVKKSAKDVSDTSSAS |
| | SNEKERKSFKLHNVNLDIRQGAFVVITGSIGSGKSSLLHALDGAMKKLSGDVYVNGSLLMCGTP |
| | WIQSASLRENILFGSTYDEKWYKEVIRACSLESDFDILPAGDLTEIGERGITLSGGQKARVCLARTV |
| | YANSSIILLDDVLSAVDAKVGKHIMSECIMGILKGKTRVLATHQLSLISEAEHVIFLNGDGTISRGTF |
| | EELKSTNSAFKALMEHNRKSEENDEDESEPASEVEASEKELIKRQLTKQTTTQVSSDSVEKVELDG |
| | KLYDEEEKSVNAIGWDVYGRYILTGVQGFKFNWLLFVILCLCILGTFMSLFTNNWLSFWISRKFD |
| | QPAGFYIGFYAAFTGLAVILMVFQFCSIIFVMNRASRILNIKALGKILHVPMSFMDTTPMGRILNR |
| | FTKDTDTLDNEIGDRVGMVVNFTFEIWGVIIMCIIYMPWFAIAVPFIVAVFIIMANFYQASGREV |
| | KRLEAVQRSHVYNNFNESLTGMPTIKAFKSIQRFLNKNVATINKMDEAYFVTVANQRWLDTYLS |
| | LLATLFALLIALLCVCRVFDIGASAVGLLVSYVLQISGLISMLVVVFTQVEQDMNSAERVMDYVYKI |
| | PQERPYEISETRPPPEWPQNGEIRFINVDFAYREGLPLTLRNINADIKPHEKIGICGRTGAGKSSIM |
| | VALFRIAELTSGSIMIDGIDVSTLGLHELRSNLSIIPQDPVLFKGTIRSNLDPFETKTDDELWDTLRRA |
| | DIIDAASLEHVKTQRVGDDDFHKFHLDNEVDDEGENFSLGEKQLVAFARALVRDTKIIVLDEATSS |
| | VDYATDSKLQKAIVREFSDRTILCIAHRLKTILHYDRVIVMEQGEIKEFDTPSHLYNSTGTIFRQMC |
| | DKSGISKEDFYEW |
| SEQ ID | ATGTCCGAACCACCAAGACAGAAGAGAATTTTGTCTTGGGCTTTGTCTAAAAAGGTCCCACC |
| NO: 939 | AATTACTCAAGAAGAGGACAGATTGGAATACCCATTCAAGAGAGCTAACATCCTGTCCAAGA |
| | TTTTCTTCTCTTGGTTGGATCCTTTGTTGCACAAGGGTTATAGAAGAACTTTGGAACCTGAAG |
| | ATTTGTGGTACTTGACCGATGAATTGAAGTTGGAACATTACTACTCCGTTTTCTTGGCTCAAT |
| | TCGAACCAGATTTGGCTGCTAGAAGAGAAGCTCATTTGGAAGCTAAATGTAAGGCTAGAGG |
| | TGAAACTTTCGAAACTTCTACTGTTACCGAAGATGAGGATTTGGCCGATTTTGTTTATCCATG |
| | GCCAAAGTTCGGTCTGATCTTGTTGAAAACTTTCTTCAGACAATACGTTGGTGCCTGCGTTTT |
| | GAAAACTATTGGTGATTTGGCTTCTACTACTGCTCCCTTGTTGCAAAAAGCTTTGATTAACTA |
| | CGTTACCAAGAGAGCCAAAGGTTTGGAGCCAAATGTTGGTACTGGTGTTGGTTATGCTATTG |
| | GTTGTGCTTTGTTCGTTACTTTGGAAGGTTTGATGGTCAACCATTACTTCTACCATGCTATGGT |
| | TACTGGTTCTCAAGTTAAGGCTATTCTGACCAAGTTCATGTTGGAAAAGTCTTTCAGACAAAC |
| | CGGTAGATCCAGACATGATTTTCCAACTGGTAAGGTCAACTCCATTATGGGTACTGATTTGG |
| | CCAGAATTGATTTCGCAATTGGTTTCTTGCCATTCTTGTTCTGTTTTCCAGTTCCAGCTATTGTC |
| | TCCATCGTTTTGTTGATCATCAATATCGGTCCATCCTCCTTGGTTGGTATTGCCATTTTCTTTTT |
| | GGCTTTGATTGCTTTGGGTTCCACCATCAAAAGATTGATGTTCTTCAGATTGAGGGCCAACAA |
| | GTTTACTGATGGTAGAGTCAATTTGGTCAAAGAGCTGTTGAAGAACTTCAAGATGATCAAGT |
| | ACTACTCCTGGGAACCATCTTACGTTAAGAACATTGAAGAAACCAGAACCGCCGAAATGCAC |
| | AATGTTTTCTTGATGCAAATCATGAGGAACATCATGGTTGCTTTCGCTATTGCTTTGCCAACT |
| | GTTTGCTCTATGATTTCCTTTTTGGTCTTGTACGGCATCAACTCCTCTAGATCTGTTGCTGATA |
| | TTTTCTCCTCCTTGACCTTGTTTCAAGTCTTGGCTATGCAATTGATCATGGTCCCATTGGCTTT |
| | GGCATCTGGTTCTGATGCTTTAATTGGTATCAGAAGAGTCTTGGAATTCGTTTGCTCTGGTGA |
| | TATCGATGAAGAAGATTCCCAAGTCGAACTGTCCTTGATCAAAGAAAAGATGGAATCCTCTG |
| | GTTCCGTTTTGAGAGTTGTTAATGCTTCTTTCGAATGGGAAACCTTCGATGCTGACGAAGAA |
| | GATATTGCTTCTACCAACGAATCCGTGTCTGAAAACGAAAGAAAACCAGATCCATCCTTGGA |
| | AGGTCTAGAATCTACATCTTTTCCAGGCTTGAACAACATCAACCTGGATATTAGAAAGGGTG |
| | AATTCGTTGTTGTCACCGGTTTGATTGGTTCTGGTAAATCTTCTTTGTTGTACGCCTTGTCTGG |
| | TTTCATGCATAGAACTCAAGGTCATGTTGCTACAATCGGTGATTTGTTGTTGTGTGGTAATCC: |
| | CTGGATTCAAAATGCTACCGTTAAGGACAATATCAGCTTCGGTATGCCATTTGACCAACAAA |
| | AGTACGATAACGTCATTCACGCTTGTTCATTGGAAGCAGATTTGGATTTGTTGCCAGCTGGT |
| | GATCATACTGAAGTTGGTGAAAGAGGTATTACTTTGTCTGGTGGTCAAAAGGCTAGATTGAA |
| | TTTGGCTAGAGCTGTTTATGCCGATAGAGACATTATCTTGTTGGACGATATTTTGTCCGCTGT |
| | TGATGCTAGAGTTGGTAAGCACATTATGGATGAATGTTTGTTGGGTCTGTTGAAGGATAAGA |
| | CCAGATTATTGGCTACCCATCAGTTGTCTTTGATTTCTGCTGCTGATAGAGTCATCTTCTTGAA |
| | CGGTGATGGTTCTATTGATGTAGGTACTACCGCTGAATTGCTGGCTAGAAATGAAGGTTTTA |
| | CCAAGCTGATGGAATTCAGCACTCAAGAAAAGAACGATACCACTACTGAATCTGGTGAAGCT |
| | GCTCATTCAGGTCCAGAATTGGAAGATGAGAAAGAATTGATCAGAATCCAGACCTTGACTAA |
| | GTCTTTGGCTGAAGCTGAATCTAACTCTGACTACCAACATAAGGATGCTGATGGTGTTTTGAT |
| | GCAGTTGGAAGATAGAGCCGTTAACGCTATTGAATTGGGTGTTTACGGTAAATACTTGAAGT |
| | TAGGTGCTGGTGCTTTTGGTATTGGTATTATCCCTTTGTTGTTAGGTTTGGTTGCTTGCTCTGT |
| | CTTTTGTTCTTTGTTCACTAACACCTGGTTGACTTTCTGGACTGAAAAGAAATTCGATAGGTCC |
| | AACGGTTTCTTTATCGGTATCTACGTTATGTTCACCATGCTGACTATCGTTTTCATGGTCTTGG |
| | AGTTCAGCTTGTTGGTTTACTTGACTAATACTGCCTCTAGGCTGTTGAACATCTACGCTATTA |
| | GAAGGTTGATGCACGTTCCAATGTCTTTCATGGATACAACTCCAATGGGTAGAATCTTGAAC |
| | AGATTCACTAAGGATACCGATGTCTTGGATAACGAATTGCCAGAACAAATTAGGTTGTTGGT |
| | TCATTTCACCGGTACTATCACTGGTATTTTGGTTTTGTGCATCATCTACTTGCCATGGTTCGCT |
| | ATTTCTGTTCCAATTTTGGCCTTCTGCTATATTGCTTGCGCTTCTTATTATCAAGCTTCCGCTAG |
| | AGAAGTCAAGAGAATTGAAGCTTTACAGAGGTCCTTCGTCTACTCCAATTTTAACGAAACATT |
| | GCAAGGCATGGAAGTTATTACTGCTTACAAAGCTGAGAAGAGGTTTATCGCAAGAAACGAT |
| | GCATTGATCGACAAGATGAACGAAGCTTACTACTTGACTTTCGCTAACATGAGATGGTTGTC |
| | CATTAGAATTGATGTTTTGGCTGCAGTCTTGGTCTTGATTGTCTCTTTGTTATGCGTCATGAG |
| | AGTGTTCCATATTTCTCCAGCTTCAGTCGGTTTGTTATTGTCTTACACTTTGAACATTGCCGGC |
| | ATGATGTCTATGTTGTTGAACGTTTCTACCCAGATCGAAAACGAGATGAACTCTGTTGAAAG |
| | ATTGGAGTACTACGGTTTCAGAGTTGTACAAGAAGCCCCATTCAAAATCTCTGAAAAGACTC |
| | CACCACCAGAATGGCCACACGATGGTAGAATTCAGTTTGAAAATGTTACCTTGTGCTACAGA |
| | CAAGGTTTACCAGCTGTTCTGAAGAATTTGAACATGGATGTTAAGGGTGCCGAAAAGATTGG |
| | TATTTGCGGTAGAACTGGTGCAGGTAAGTCATCTATTATGACTGCCTTGTATAGATTGGCCG |
| | AAATGGAATCAGGTGGTAGGATTTTGATCGATGATATTGACATTTCTACCTTGGGCTTGCAC |
| | GATTTGAGATCCAGATTGTCTATTATCCCACAAGATCCAGTTTTGTTCAGAGGTTCTATTAGA |
| | GGTAACTTGGATCCATTCCATGAACACAAAGACGAATTATTGTGGGATGCCTTGAGAAGATC |
| | CGGTTTAATTGAAGGTTCTAAGTTGGATCAAGTGAAGCACCAAACTTTGGATGACGAAAACT |
| | TGCATAAGTTCCACTTGGGTCAAAACGTTGAAGATGATGGTACAAACTTCAGCTTGGGTGAA |
| | AGACAATTGCTAGCTTTAGCTAGAGCTTTGGTGAGAAACTCTAAGATCTTGATTTTGGATGA |
| | AGCCACCTCCTCTGTTGATTACGAAACTGATTCTAAGATCCAAACCACCATCTCTACTGAATTT |
| | GCTGGTTGTACTATTATGTGCATTGCCCACAGATTGAAAACCATCGTTAACTACGATAGGATC |
| | CTGGTTTTGGATAAGGGTGAAATTTCCGAATTTGATAAGCCATGGGCCTTGTTCCAAGATGA |
| | ATCTACTATTTTCAGGCAGATGTGTAACAAGTCTGGTGTTGTTGCAGAAGATTTCGAGAAGC |
| | AAAACTAA |
| SEQ ID | MSEPPRQKRILSWALSKKVPPITQEEDRLEYPFKRANILSKIFFSWLDPLLHKGYRRTLEPEDLWYL |
| NO: 940 | TDELKLEHYYSVFLAQFEPDLAARREAHLEAKCKARGETFETSTVTEDEDLADFVYPWPKFGLILLK |
| | TFFRQYVGACVLKTIGDLASTTAPLLQKALINYVTKRAKGLEPNVGTGVGYAIGCALFVTLEGLMV |
| | NHYFYHAMVTGSQVKAILTKFMLEKSFRQTGRSRHDFPTGKVNSIMGTDLARIDFAIGFLPFLFCF |
| | PVPAIVSIVLLIINIGPSSLVGIAIFFLALIALGSTIKRLMFFRLRANKFTDGRVNLVKELLKNFKMIKYY |
| | SWEPSYVKNIEETRTAEMHNVFLMQIMRNIMVAFAIALPTVCSMISFLVLYGINSSRSVADIFSSL |
| | TLFQVLAMQLIMVPLALASGSDALIGIRRVLEFVCSGDIDEEDSQVELSLIKEKMESSGSVLRVVNA |
| | SFEWETFDADEEDIASTNESVSENERKPDPSLEGLESTSFPGLNNINLDIRKGEFVVVTGLIGSGKS |
| | SLLYALSGFMHRTQGHVATIGDLLLCGNPWIQNATVKDNISFGMPFDQQKYDNVIHACSLEADL |
| | DLLPAGDHTEVGERGITLSGGQKARLNLARAVYADRDIILLDDILSAVDARVGKHIMDECLLGLLK |
| | DKTRLLATHQLSLISAADRVIFLNGDGSIDVGTTAELLARNEGFTKLMEFSTQEKNDTTTESGEAA |
| | HSGPELEDEKELIRIQTLTKSLAEAESNSDYQHKDADGVLMQLEDRAVNAIELGVYGKYLKLGAG |
| | AFGIGIIPLLLGLVACSVFCSLFTNTWLTFWTEKKFDRSNGFFIGIYVMFTMLTIVFMVLEFSLLVYL |
| | TNTASRLLNIYAIRRLMHVPMSFMDTTPMGRILNRFTKDTDVLDNELPEQIRLLVHFTGTITGILVL |
| | CIIYLPWFAISVPILAFCYIACASYYQASAREVKRIEALQRSFVYSNFNETLQGMEVITAYKAEKRFIA |
| | RNDALIDKMNEAYYLTFANMRWLSIRIDVLAAVLVLIVSLLCVMRVFHISPASVGLLLSYTLNIAG |
| | MMSMLLNVSTQIENEMNSVERLEYYGFRVVQEAPFKISEKTPPPEWPHDGRIQFENVTLCYRQ |
| | GLPAVLKNLNMDVKGAEKIGICGRTGAGKSSIMTALYRLAEMESGGRILIDDIDISTLGLHDLRSRL |
| | SIIPQDPVLFRGSIRGNLDPFHEHKDELLWDALRRSGLIEGSKLDQVKHQTLDDENLHKFHLGQN |
| | VEDDGTNFSLGERQLLALARALVRNSKILILDEATSSVDYETDSKIQTTISTEFAGCTIMCIAHRLKTI |
| | VNYDRILVLDKGEISEFDKPWALFQDESTIFRQMCNKSGVVAEDFEKQN |
| SEQ ID | ATGTCCGACTACGACTTGGAAGAAAACCATTTGGTTAGACAGAACAGGCTGTTGTCCTCTTT |
| NO: 941 | GTTCTCTAAAGAATTGCCTCCAATTCCAGAAGATGACGAAAGACCAGAACATCCAGAAAGAG |
| | ATGCTAACTTCTTCAGCAAGATTTTCTTCTGGTGGATGATCCCAGTTATGAACACTGGTTACA |
| | AGAGAACTTTGACCCCAAAGGATTTGTTCACCTTGTCCGATGATATTAAGGTTGAAACTATG |
| | GCTGCTAGGTTTATGGCTATTTTCACCTCTGATGTTGAAAGGGCCAAATTGAAACACGTTAA |
| | GAAAAAGTGTAAGAAGAGGGGCGAAACCTTGGAATCTTCTTCTGTTGATTTCGATACCGACG |
| | TCGAAGATTTTAAGGTTTCCCCAATTATGTTCTTCTTCACCATCTGGAAAACCTACAAGTACCA |
| | ATACTTTGCTGCTTCTGTTTGCTTGGCTATTGCTAATTCTGCTCAAGCTGTTAACCCTCTGTTG |
| | TTCAAAAAGTTGATTACCTACGTTGGTTTGAAGGCCTACGGTATTGAACAAGGTGTTGGTAA |
| | AGGTGTCGGTTATGCTATTGGTTCTTGCTTGATTGAATTCTTGGGTGCCGTTTTGTTCAACCA |
| | CTTCTTTTACAAAGCTATGATGACCGGTGCTGAAACTAAGGGTGTTTTGACAAAAGCTTTGTT |
| | GGAGAAGTCCTTCAGATTGTCCGCTGAATCTAAACATAAGTTCCCAGTTGGTAAGATCACCTC |
| | TATGATGGGTACTGATTTGTCCAGAATTGATTTGGCCTTGGGTTTACAACCATTCATCTTCGTT |
| | TTTCCCATTCCAATCGTTATCTCCATTGCCATCTTGATCGTTAACATTGGTGCTGTTGCTTTGAT |
| | TGGTATCGGTGTTATGTTGTTGTTCATGGCTGTTATTGGTGGTACTACTGCTAAGTTGTACTC |
| | CTATAGAACTAAGGCTAACAAGTACACCGATATCAGAGTCTCTTACATGAAGGAAGTCCTGA |
| | ACAACTTGAAGATGATCAAGTTCTATTCTTGGGAGCCACCTTACTACGAAAACATTTCTTCTA |
| | CTAGGACCAAAGAGATGGACATCATCTACAACATGCAAACCTTGAGATCTATCGTTACTGCTT |
| | TGGCTATGTCTTTGACTGGTTTTGCTTCTTTGGTTGCCTTCTTGGTTTTGTTCGCTGTTGATAAT |
| | GATAGAAAGAACCCAGCCTCCATCTTCTCCTCAATTTCTTTGTTTAACGTCTTGCTGACCCAGG |
| | TCTTTATGTTGCCAATGGCTTTAGCTACTTCTGCTGATGCTTTTGCTGGTGTAGGTAGAGTTTC |
| | TACTTTCTTGACTACTGGTGAAGTCGACCCAAAAGAATTGGAAACTGATATTTCAGCCGACGT |
| | CTTGCAGAGAATGGACAAAGAAGATGTTGTTATCGAAGTGAACAACGCCTCTTTTGAATGGG |
| | AGATTTTCGAAGATATCGAAGAGAAGGACCCAAAGAAAGAGAAAGAGGAAAAGAAAAAGG |
| | CTAAAAAGGCCGCCAAAGAGACTAAGAAATTGGCTAAACAAGCCAAGAACTCCCAAACTATT |
| | ACTCCATCCGAAGAAGAGTTGTCCAAGATTGATTCTCCAAAGTTCACCGAGAAAGAATTGTC |
| | TACCGAATCCAAGTCTGTTGAGGAAAAAGTTTTCGCCGGTCTGAACAACATTAACCTGTCTAT |
| | TGCCTTGTCCGGCTTTATGAAGAAAACTTCCGGTGAAGTTCTGGTCAGTTCATCTTTGTTGTT |
| | TAAGAAGAACGAGTTCGTTGTTATCACCGGTATGATTGGTTCTGGTAAGACTTCTTTGTTGAA |
| | GTGTGGTTACCCATGGATTCAAAACACTACCGTTAGAGAGAATATCGTTTTCGGTTCTGAAT |
| | GGGACGAAGAAAAGTACAACAGAGTTATTTTCGCCTGCTCTTTGGAATCCGACATTGAAATT |
| | TTGCCAGGTGGTGATTTGACCGAAATTGGTGAAAGAGGTATTACTTTGTCTGGTGGTCAAAA |
| | GGCTAGAATCAACTTAGCTAGAGCTGTTTATGGTGGTAGGGAAATCATTTTGATGGATGATG |
| | TTTTGTCTGCCGTTGATGCTAGAGTTGGTAAACACATTATGAACAACTGCATCCTGGATCTGT |
| | TGAAGGATTCTACTAGAATTTTGGCTACCCACCAGTTGTCTTTGATAGATTCTGCAGATAGGG |
| | TCATCTTCCTAAATGGTGATGGTTCTATTTCCGTCGGTACAAACGAAGAATTGCAAAAGTCTA |
| | ATCCAGGTTTCGCTGCTTTGATGGCTCATAATGCTAAAACTGAAGAGGACGATGAAGATGAG |
| | AAGATCGATGTTGATTTGGACAAGCAAAAGTTCGAAGAACACCACGAAGTTGAGAAAGAGT |
| | TGATTCAGAGACAAGTTACTAGAGCCTCTGCTGTTGATGAAGAAGCTATTAGAAAGGACTAC |
| | AACAAGAACGTGGAAGAAGATGGTCATTTGATCGAAGATGAGGATAGAGGTGTTAACGCTA |
| | TTGCTTTGGATGTTTACTTGACCTACGTTAAGTTAGGTTCCGGTAAGTATACTGCTTGGGGTA |
| | TAGTTCCACCTATGTTAGTTTTTATGGCTTTGGCCACTTTCTGCCAAATTTTCACTAATACCTG |
| | GTTGTCTTTTTGGACCGAGAACAAATTCTCTGGTAAGGATGATAACTTCTACATCGGTATCTA |
| | CGTTATGTTCACCGTCTTGTCTTTTGTTTTCCTGGCTTTGGAATTCATGTCCTTGGTTTACATGA |
| | CTAATACCGCTGCTGTCAAGTTGAATATTGCTGCTGTTCAAAAGGTTCTGAAGGTTCCTATGG |
| | CTTTTATGGATACAACTCCAATGGGTAGAATCTTGAACAGGTTCACTAAGGATACTGACGTTT |
| | TGGATAACGAAATCGGTGAACAGATTAACTTCGCCTTGTTCATGTTGTCTAACGTTGTTGGTA |
| | TTATCATCTTGTGCATTATCTACTTGCCATGGTTTGCTATTGCTGTCCCATTTTTGGGTTTCATG |
| | TTTATCGCTGTCTCCAACTATTATCAAGCTTCCGCTAGAGAAATCAAGAGATTGGAAGCTGTT |
| | TCTAGGTCCTTTGTCTACAACAACTTTAACGAAGTCTTGAACGGTATCAACACCATTAACGCT |
| | TATAAGGCCGAATCCAGATTTGTTGCTAAGAACGACAGATTGATCAACGGTATGAACGAGTC |
| | TTACTACTTGACCATCGGTAATCAAAGATGGTTGGGTATTCAGATGAACATTATTGCCGTCCT |
| | GTTCTCCTTGTTGATTGCTTTGTTATGCGTGAACAGGGTTTTCAAGATTTCTCCAGCTTCTGTC |
| | GGTCTGTTGTTGTCTTATGTATTTTCTATCGGTGGCACCCTGTCCATGTTGATTAGAACTTTTA |
| | CACAGGTCGAGAACGAGATGAATTCCGTTGAAAGAATCTCCTACTACTCGTTCTCTTTGCCAC |
| | AAGAAGCTCCATCTTACATTACTGAAAATTCTCCACCACCAGAATGGCCAGCTAAAGGTGAA |
| | ATTCATTTCAAGGATACCTCATTGGCTTACAGACCAGGTTTGCCATTGGTTTTGAAGAACTTG |
| | AACTTCTCCATTAAGGGCTCCGAAAAGATTGGTATTTGTGGTAGAACTGGTGCCGGTAAATC |
| | TTCTATTATGACTGCCTTGTACAGGCTGTCTGAATTGGATGGTGGTTCAATTGTCATTGATGA |
| | CATCGATATTTCTACCTTGGGCTTGCATGATTTGAGGTCCAAGTTGTCTATTATCCCACAAGA |
| | TCCAGTCATGTTCAGAGGTACTATTAGGAAAAACTTGGATCCATTCGATCAATCCACCGATGA |
| | TCAATTGTGGGGTGCTTTAGTTAGAACAGGTTTGGTTGAAGCTGATAGATTGGATGTTGTAA |
| | AGGCTCAAGTTAAGGTGCAGAAAGAAGATAAGTCTGATCACGGTGACAACAACAATGGTGC |
| | TGATAAGAAAGGTGCCGAAGAAGGTTCTATCTTGCATAAGTTTCACTTGGATCAAATGGTTG |
| | AGGATGAAGGTGTCAATTTCTCATTGGGTGAAAGACAATTGATTGCCTTTGCTAGAGCCTTG |
| | GTCAGAAATTCCAAGATTTTGATTTTGGACGAAGCCACCTCTTCCGTTGATTACGAAACTGAT |
| | GCTAAGATCCAAAACTCCATCGTTAATGAATTCGCCGATTGCACCATTTTGTGCATTGCTCAT |
| | AGACTGAAAACCATCATCAACTACGATAAGATCCTGGTCTTGGATAAGGGTGAGATCAAAG |
| | AGTTTAATACCCCTTGGAACTTGTTTAAGACCAAGGACTCCATTTTCCAACAGATGTGCATTA |
| | AGTCCAACATCGTTGAAGAGGATTTCCACAGAGTGTCTAAGTTCTGA |
| SEQ ID | MSDYDLEENHLVRQNRLLSSLFSKELPPIPEDDERPEHPERDANFFSKIFFWWMIPVMNTGYKRT |
| NO: 942 | LTPKDLFTLSDDIKVETMAARFMAIFTSDVERAKLKHVKKKCKKRGETLESSSVDFDTDVEDFKVS |
| | PIMFFFTIWKTYKYQYFAASVCLAIANSAQAVNPLLFKKLITYVGLKAYGIEQGVGKGVGYAIGSCL |
| | IEFLGAVLFNHFFYKAMMTGAETKGVLTKALLEKSFRLSAESKHKFPVGKITSMMGTDLSRIDLAL |
| | GLQPFIFVFPIPIVISIAILIVNIGAVALIGIGVMLLFMAVIGGTTAKLYSYRTKANKYTDIRVSYMKEV |
| | LNNLKMIKFYSWEPPYYENISSTRTKEMDIIYNMQTLRSIVTALAMSLTGFASLVAFLVLFAVDND |
| | RKNPASIFSSISLFNVLLTQVFMLPMALATSADAFAGVGRVSTFLTTGEVDPKELETDISADVLQR |
| | MDKEDVVIEVNNASFEWEIFEDIEEKDPKKEKEEKKKAKKAAKETKKLAKQAKNSQTITPSEEELS |
| | KIDSPKFTEKELSTESKSVEEKVFAGLNNINLSIKKNEFVVITGMIGSGKTSLLNALSGFMKKTSGEV |
| | LVSSSLLLCGYPWIQNTTVRENIVFGSEWDEEKYNRVIFACSLESDIEILPGGDLTEIGERGITLSGG |
| | QKARINLARAVYGGREIILMDDVLSAVDARVGKHIMNNCILDLLKDSTRILATHQLSLIDSADRVIF |
| | LNGDGSISVGTNEELQKSNPGFAALMAHNAKTEEDDEDEKIDVDLDKQKFEEHHEVEKELIQRQ |
| | VTRASAVDEEAIRKDYNKNVEEDGHLIEDEDRGVNAIALDVYLTYVKLGSGKYTAWGIVPPMLVF |
| | MALATFCQIFTNTWLSFWTENKFSGKDDNFYIGIYVMFTVLSFVFLALEFMSLVYMTNTAAVKL |
| | NIAAVQKVLKVPMAFMDTTPMGRILNRFTKDTDVLDNEIGEQINFALFMLSNVVGIIILCIIYLPW |
| | FAIAVPFLGFMFIAVSNYYQASAREIKRLEAVSRSFVYNNFNEVLNGINTINAYKAESRFVAKNDRL |
| | INGMNESYYLTIGNQRWLGIQMNIIAVLESLLIALLCVNRVFKISPASVGLLLSYVFSIGGTLSMLIR |
| | TFTQVENEMNSVERISYYSFSLPQEAPSYITENSPPPEWPAKGEIHFKDTSLAYRPGLPLVLKNLNF |
| | SIKGSEKIGICGRTGAGKSSIMTALYRLSELDGGSIVIDDIDISTLGLHDLRSKLSIIPQDPVMFRGTIR |
| | KNLDPFDQSTDDQLWGALVRTGLVEADRLDVVKAQVKVQKEDKSDHGDNNNGADKKGAEEG |
| | SILHKFHLDQMVEDEGVNFSLGERQLIAFARALVRNSKILILDEATSSVDYETDAKIQNSIVNEFAD |
| | CTILCIAHRLKTIINYDKILVLDKGEIKEFNTPWNLFKTKDSIFQQMCIKSNIVEEDFHRVSKF |
| SEQ ID | ATGATGGACCAAAACCACCACTCTGATAAGACTGCTGAAGCTCAACCATCTGAAAAGAGAAA |
| NO: 943 | GACTAGAAACTGCTGTAACGGCTTCAAGATGTTTTTGGCTGCTTTGTCCTTGTCCTTCATTTCT |
| | AAAGCTTTGGGTGCCATCATCATGAAGTCCTCTATTACCCAAATCGAAAGAAGGTTCGACAT |
| | CTCTTCTTCTACCGTTGGTTTGATTGATGGTTCCTTCGAAATTGGTAACTTGCTGGTTATCGTG |
| | TTCGTTTCTTACTTTGGTGCTAAGTTGCATAGGCCAAAGTTGATTGGTATTGGTTGTATCGTT |
| | ATGGGCACCGGTTCTATTTTGACTGCTTTACCACATTTCTTCATGGGCTACTACAGATACTCTA |
| | AGGATACCACTTTTAACCCCTCTGAAAACTCTACTTCTAACTTGCCAACCTGCTCTATTAACCA |
| | GTCTTTGTTGCCAAACAGAACCTCCTTGGAAGTTGTTGAAAAAGGTTGGGAAAAAGACTCCG |
| | GTTCTTATATGTGGATCTACGTTTTGATGGGTAACATGTTGAGAGGTATTGGTGAAACTCCA |
| | ATTGGTCCATTGGGTATCTCTTACATTGATGATTTTGCCGAAGAAGGCCACTCTTCATTATACT |
| | TGGGTAATTTGTTGGCCGTTACCATGATTGGTCCAATTATTGGTTTCGTCTTGGTGTCCCAAT |
| | TCTCCAAGATGTATGTTGATATCGGTTACGTTGACTTGTCCACCATTAGAATTACTCCAAAGG |
| | ATTCTAGATGGGTTGGTGCTTGGTGGTTGGGTTTCTTAGCTGCTGGTTTGATCTCCATTATTA |
| | GCTCTATCCCTTTCTTCTTCCTGCCAAAAACTTTGGACAAACCCAACAAAGAAAAGAAGGCTT |
| | CCGTTTCCTCTATCTCTTTGCCAATGAAGAACGAAGAAAAGTCCCAAATGGCTTCTTTGACTA |
| | AGACTGGTCAAAACGTTACTGATACCATTACCGGTTTCTTGCAGTCCTTGAAATCCATTTTGA |
| | CTAACCCCTTGTACGTCTTGACTTTGTTTTTGACCTTGTTGCAAGCCTCCTCTCATATTGGTTCT |
| | GTTACTTACGTTTTCAAGTACGTCGAACAACAGTACGGTAATTCTGCTTCTGAAACCTCCATTT |
| | TGTTGGGCTTCATTACCATTCCATCATTGGCTGCTGGTATGTTTACAGGTGGTTTTATTGTCAA |
| | GAAGTTCAAGTTCACCTTGGTTGGTATTGCCAAGTTCTCTTTGTGTACCCATTTGATGTCTTTC |
| | TTGTTCCACTTGTTGAACTTCGCTTTGATCTGCGAAAACAAATCTGTTGCAGGTTTGACTTTGA |
| | CCTACGATGGTAATACTCCAGTTGCTTCTCATGTTAACGTTCCATTGTCTTACTGTAACTCTGA |
| | TTGCAACTGTGATCCAACTCAATGGGAACCATTGTGTGGTTCAAATGGTATTACTTACATCTC |
| | TCCATGTTTGGCTGGTTGTACATCTTCTACTGGTTCTAAGAAGTCCATCGTTTTCCATAACTGT |
| | TCTTGCGTTGAAGAAAACGGTTTCCAGTCTAAGAACAACTCTATGAAGTTGGGTGAATGCCC |
| | AAAATCTGATGATTGCAGAAGAAAGTTCTACATCTACATCGTCGTTCAGATCTTGACCTTATT |
| | TTCTGCTGCTTTGGGTTCCACCTCTAACATTATGTTGATCTTCAAGAACGTGGAACCCGAGTT |
| | GAAATCTTTGGCTATGGGTTTTCATTCCTTGACCATCAGAACTTTAGGTGGTATTCCAGCTCC |
| | AATCTATTTCGGTGCTTTAATTGATAGAGCTTGCATGAAGTGGTCGATCAACAATTATGGTAA |
| | GCAAGGTTCTTGCAGGATCTACAACTCTTTGTTGTATGGTCATACTTTCTTCGGTTTGACTACC |
| | GGTTTGAAATTTCCAGCTTTGGTTTTGTTCGTCGTTTTGATTTTCGCTATGAAGAAGAAGTAC |
| | CAAGGCAAGGATATTAAGGCCTCAGAAAACGAAACTAAGGCTGTTAATGAAGCTAACTCCG |
| | GCTTGTTGATTAACGATGCTAAAGTTGATCACGAAACCCACATCTGA |
| SEQ ID | MMDQNHHSDKTAEAQPSEKRKTRNCCNGFKMFLAALSLSFISKALGAIIMKSSITQIERRFDISSS |
| NO: 944 | TVGLIDGSFEIGNLLVIVFVSYFGAKLHRPKLIGIGCIVMGTGSILTALPHFFMGYYRYSKDTTFNPS |
| | ENSTSNLPTCSINQSLLPNRTSLEVVEKGWEKDSGSYMWIYVLMGNMLRGIGETPIGPLGISYID |
| | DFAEEGHSSLYLGNLLAVTMIGPIIGFVLVSQFSKMYVDIGYVDLSTIRITPKDSRWVGAWWLGFL |
| | AAGLISIISSIPFFFLPKTLDKPNKEKKASVSSISLPMKNEEKSQMASLTKTGQNVTDTITGFLQSLKS |
| | ILTNPLYVLTLFLTLLQASSHIGSVTYVFKYVEQQYGNSASETSILLGFITIPSLAAGMFTGGFIVKKFK |
| | FTLVGIAKFSLCTHLMSFLFHLLNFALICENKSVAGLTLTYDGNTPVASHVNVPLSYCNSDCNCDPT |
| | QWEPLCGSNGITYISPCLAGCTSSTGSKKSIVFHNCSCVEENGFQSKNNSMKLGECPKSDDCRRK |
| | FYIYIVVQILTLFSAALGSTSNIMLIFKNVEPELKSLAMGFHSLTIRTLGGIPAPIYFGALIDRACMKW |
| | SINNYGKQGSCRIYNSLLYGHTFFGLTTGLKFPALVLFVVLIFAMKKKYQGKDIKASENETKAVNEA |
| | NSGLLINDAKVDHETHI |
| SEQ ID | ATGCAACCAGTTTACCCAGAGGTTAAGCCAAATCCATTGAGAAATGCTAACTTGTGCTCCAG |
| NO: 945 | AGTTTTCTTTTGGTGGTTGAATCCTTTGTTCAAGATCGGTCATAAGAGAAGATTGGAAGAGG |
| | ATGATATGTACTCCGTTTTGCCAGAAGATAGATCTCAACATTTGGGTGAAGAATTGCAAGGT |
| | TACTGGGATCAAGAAGTTTTGAAGGCTGAAAAGGATGCTAGAGAACCATCTTTGACTAAGG |
| | CCATTATTAAGTGCTACTGGAAGTCCTATGTTGTCTTGGGTATTTTCACCTTGATCGAAGAAT |
| | CTACCAGAGTTGTTCAGCCAATTATCTTGGGTAAGATCATCGGTTACTTCGAAAACTACGATC |
| | CATCTGATTCTGCTGCATTATATGAAGCTCATGGTTATGCTGGTGTTTTGTCTGCTTGTACTTT |
| | GGTTTTGGCTATCTTGCATCACTTGTACTTCTACCATGTTCAATGTGCTGGTATGAGATTGAG |
| | AGTTGCTATGTGTCATATGATCTACAGAAAGGCCTTGAGGTTGTCTAATTCTGCTATGGGTAA |
| | AACTACTACCGGTCAAATCGTTAACTTGTTGTCCAACGATGTTAACAAGTTCGACCAAGTTAC |
| | CATCTTCTTGCATTTCTTGTGGGCTGGTCCATTGCAAGCTATAGTTGTTACTGCTTTGTTGTGG |
| | ATGGAAATCGGTATTTCTTGTTTAGCTGGTATGGCCGTTTTGATCATTTTGTTGCCATTGCAAT |
| | CTTGTATCGGCAAGTTGTTCTCTTCATTGAGATCAAAGACTGCTGCTTTCACCGACACTAGAA |
| | TTAGAACTATGAACGAAGTTATCACCGGCATCAGAATCATTAAGATGTACGCTTGGGAAAAG |
| | TCCTTCGCTGATTTGATTACCAACCTGAGAAGAAAAGAGATCTCGAAGATTCTGAGGTCCTCT |
| | TATTTGAGAGGTATGAACTTGGCTTCTTTCTTCGTTGCCTCCAAAATCATCGTTTTCGTTACTT |
| | TCACTACCTACGTCTTGTTGGGTAACGTTATTACTGCTTCTAGAGTTTTCGTTGCCGTTTCATT |
| | ATATGGTGCTGTTAGATTGACTGTCACCTTGTTTTTCCCATCTGCTGTTGAAAAAGTTTCCGAA |
| | GCCTTCGTTTCCATCAGAAGAATCAAGAACTTTCTGTTGTTGGACGAAATCACCCAATTGCAT |
| | TCTCAATTGCCATCTGATGGTAAGATGATCGTTAACGTTGAAGATTTCACTGCTTTCTGGGAC |
| | AAAGCTTCTGATACTCCAACTTTACAAGGTTTGTCTTTCACTGTTAGACCAGGTGAATTATTG |
| | GCAGTTGTTGGTCCAGTTGGTGCTGGTAAATCTTCTTTGTTGTCTGCTGTTTTAGGTGAGTTG |
| | CCACCATCTCAAGGTCAAGTTTCTGTTCACGGTAGAATTGCTTACGTTTCTCAACAACCATGG |
| | GTTTTCTCTGGTACAGTGAGATCCAATATTTTGTTCGGTAAGAAGTACGAGAAAGAGAGGTA |
| | CGAAAAGGTTATTAAGGCTTGCGCTTTGAAAAAGGACTTGCAGTTATTGGAAGATGGCGACT |
| | TGACTATGATTGGTGATAGAGGTACTACTTTGTCTGGTGGTCAAAAGGCTAGAGTTAATTTG |
| | GCTAGAGCTGTTTATCAAGATGCCGACATCTATTTGTTGGATGATCCATTGTCAGCTGTTGAT |
| | GCTGAAGTTTCTAGACACTTGTTCGAATTGTGTATTTGTCAAGCCTTGCACGAAAAGATCAGA |
| | ATTTTGGTTACCCACCAACTGCAATACTTGAAAGCTGCTTCCCAAATCTTGATCTTGAAAGAC |
| | GGTAAAATGGTTCAAAAGGGTACTTACACCGAGTTCTTGAAGTCTGGTATTGATTTCGGTTC |
| | CCTGTTGAAGAAAGAGAACGAAGAAGCTGAACCATCTCCAGTTCCAGGTACTCCAACATTAA |
| | GAAATAGAACCTTCTCCGAATCCTCCGTTTGGTCACAACAATCTTCTAGACCATCATTGAAAG |
| | AAGCTACTCCAGAAGGTCCAGATACCGAAAACATTCAAGTTACTTTGACCGAAGAAACCAGG |
| | TCTGAAGGTAAGGTTGGTTTTAAAGCTTACAAGAACTACTTCACTGCTGGTGCTCATTGGTTC |
| | ATCATCATCTTTTTGATCTTGGTTAACTTGGCTGCTCAAGTTGCTTATGTATTGCAAGACTGGT |
| | GGTTGTCTTATTGGGCTAATCAACAATCTGCTTTGAACGTTACTGTTAACGGTCAAGGTAATG |
| | TCACCGAAAAGTTGAATTTGAACTGGTACTTGGGTATCTACTCTGGTTTGACAGCTTCTACTG |
| | TTTTGTTTGGTATTGCCAGATCCTTGTTGGTTTTCTTCGTCTTGGTTTCCTCTTCTCAAACCTTG |
| | CACAATCAGATGTTCGAATCTATTTTGAGAGCCCCAGTTTTGTTCTTCGATAGAAATCCAATT |
| | GGCCGTATCCTGAACAGATTCTCTAAAGATATTGGTCACATGGACGACTTGTTGCCTTTGACT |
| | TTCTTGGATTTCATCCAGACATTCTTGCAAGTTATCGGTGTTGTTGGTGTTGCAGTTGCTGTTA |
| | TTCCATGGATTGCTATTCCATTGGTTCCTTTGGGTATCGTGTTTTTCGTATTGAGAAGGTACTT |
| | CTTGGAAACCTCCAGAGATGTTAAGAGATTGGAATCTACTACTAGGTCCCCAGTGTTTTCTCA |
| | TTTGTCATCTTCATTGCAAGGATTGTGGACCATTAGAGCTTACAAAGCCGAACAAAGATTCCA |
| | AGAGTTGTTTGATTCTCACCAAGACTTGCATTCTGAAGCCTGGTTTTTGTTTTTGACTACCTCT |
| | AGATGGTTCGCCGTTAGATTGGATGCTATTTGTGCTGTTTTTGTTATCGTTGTCGCTTTCGGTT |
| | CATTGATTTTGGCTAAAACTTTGGATGCTGGTCAAGTTGGTTTGGCTTTGTCTTATGCTTTAAC |
| | CTTGATGGGTATGTTCCAATGGTGTGTTAGGCAATCAGCTGAAGTTGAAAACATGATGATCT |
| | CCGTTGAAAGAGTTATCGAGTACACCGACTTGGAAAAAGAAGCTCCTTGGGAATCTCAGAA |
| | AAGACCATTGCCATCATGGCCACATGAAGGTGTTATTATCTTCGATAACGTCAACTTCTCCTA |
| | CTCTTTGGATGGTCCTTTAGTCTTGAAACATTTGACCGCCTTGATTAAGTCTAGAGAAAAGGT |
| | TGGTATCGTTGGTAGAACTGGTGCAGGTAAGTCATCTTTAATTGCTGCCTTGTTCAGATTGTC |
| | TGAACCAGAAGGTAAAATCTGGATCGATAAGATCTTGACTACCGAAATTGGCTTGCACGATT |
| | TGAGAAAGAAGATGTCCATTATTCCACAAGAGCCAGTGTTGTTTACAGGTACTATGAGGAAA |
| | AACTTGGACCCATTCTCTGAACACTCCGATGAAGAATTATGGAACGCTTTGGAAGAAGTGCA |
| | ATTGAAAGAGGCCATAGAAGATTTGCCTGGCAAAATGGATACTGAATTAGCTGAATCTGGCT |
| | CCAATTTCTCTGTTGGTCAAAGACAATTGGTTTGTTTGGCCAGAGCTATCTTGAGGAAGAAC |
| | AGGATTTTGATTATTGATGAAGCTACCGCCAACGTTGATCCAAGAACTGATGAATTGATCCA |
| | AAAGAAGATCAGGGAAAAGTTCGCTCATTGCACTGTTTTGACTATTGCCCATAGATTGAACA |
| | CCATCATCGATTCCGACAAGATCATGGTTTTGGATTCCGGTAGGTTGAAAGAATACGATGAA |
| | CCATATGTCTTGTTGCAGAACAGAGACTCTTTGTTCTACAAGATGGTACAGCAATTAGGTAAA |
| | GCTGAAGCTGCCGCTTTGACTGAAACTGCAAAACAAGTTTACTTCAAGAGGAACTACCCAGA |
| | TATTACCCATAACGGTCATGTTGTTATGAATGCCTCTTCTGGTCAACCATCTGCCTTGACTATT |
| | TTTGAAACTGCCTTGTAA |
| SEQ ID | MQPVYPEVKPNPLRNANLCSRVFFWWLNPLFKIGHKRRLEEDDMYSVLPEDRSQHLGEELQGY |
| NO: 946 | WDQEVLKAEKDAREPSLTKAIIKCYWKSYVVLGIFTLIEESTRVVQPIILGKIIGYFENYDPSDSAALY |
| | EAHGYAGVLSACTLVLAILHHLYFYHVQCAGMRLRVAMCHMIYRKALRLSNSAMGKTTTGQIV |
| | NLLSNDVNKFDQVTIFLHFLWAGPLQAIVVTALLWMEIGISCLAGMAVLIILLPLQSCIGKLFSSLR |
| | SKTAAFTDTRIRTMNEVITGIRIIKMYAWEKSFADLITNLRRKEISKILRSSYLRGMNLASFFVASKII |
| | VFVTFTTYVLLGNVITASRVFVAVSLYGAVRLTVTLFFPSAVEKVSEAFVSIRRIKNFLLLDEITQLHS |
| | QLPSDGKMIVNVEDFTAFWDKASDTPTLQGLSFTVRPGELLAVVGPVGAGKSSLLSAVLGELPPS |
| | QGQVSVHGRIAYVSQQPWVFSGTVRSNILFGKKYEKERYEKVIKACALKKDLQLLEDGDLTMIGD |
| | RGTTLSGGQKARVNLARAVYQDADIYLLDDPLSAVDAEVSRHLFELCICQALHEKIRILVTHQLQY |
| | LKAASQILILKDGKMVQKGTYTEFLKSGIDFGSLLKKENEEAEPSPVPGTPTLRNRTFSESSVWSQ |
| | QSSRPSLKEATPEGPDTENIQVTLTEETRSEGKVGFKAYKNYFTAGAHWFIIIFLILVNLAAQVAYV |
| | LQDWWLSYWANQQSALNVTVNGQGNVTEKLNLNWYLGIYSGLTASTVLFGIARSLLVFFVLVSS |
| | SQTLHNQMFESILRAPVLFFDRNPIGRILNRFSKDIGHMDDLLPLTFLDFIQTFLQVIGVVGVAVA |
| | VIPWIAIPLVPLGIVFFVLRRYFLETSRDVKRLESTTRSPVFSHLSSSLQGLWTIRAYKAEQRFQELFD |
| | SHQDLHSEAWFLFLTTSRWFAVRLDAICAVFVIVVAFGSLILAKTLDAGQVGLALSYALTLMGMF |
| | QWCVRQSAEVENMMISVERVIEYTDLEKEAPWESQKRPLPSWPHEGVIIFDNVNFSYSLDGPLV |
| | LKHLTALIKSREKVGIVGRTGAGKSSLIAALFRLSEPEGKIWIDKILTTEIGLHDLRKKMSIIPQEPVLF |
| | TGTMRKNLDPFSEHSDEELWNALEEVQLKEAIEDLPGKMDTELAESGSNFSVGQRQLVCLARAIL |
| | RKNRILIIDEATANVDPRTDELIQKKIREKFAHCTVLTIAHRLNTIIDSDKIMVLDSGRLKEYDEPYVL |
| | LQNRDSLFYKMVQQLGKAEAAALTETAKQVYFKRNYPDITHNGHVVMNASSGQPSALTIFETAL |
| SEQ ID | ATGGACACCTCCTCTAAAGAAAACGCTCATTTGTTCCATAAGAACTCTGCTCAACCTGCTGGT |
| NO: 947 | GGTCCATCTTTTACTGTTGGTTATCCATCTACTGAAGAGGCTAGACCATGTTGTGGTAAATTG |
| | AAAGTTTTCTTGGGTGCCTTGTCCTTCGTTTACTTTGCTAAAGCTTTGGCTGAAGGCTACTTGA |
| | AGTCTACTGTTACTCAAATCGAAAGGCGTTTCGAAATCCCATCTTCTTTGGTTGGTATTATCG |
| | ACGGTTCTTTCGAGATTGGTAACTTGTTGGTCATTACCTTCGTTTCTTACTTCGGTGCTAAATT |
| | GCATAGGCCAAAGATTATTGGTGCTGGTTGTTTGGTTATGGGTTTCGGTACTATGTTGATTGC |
| | TGTTCCACAATTCTTCATGGAAAAGTACTCCTACGAAAAGTACGAAAGGTACTCTCCATCTTC |
| | TAACGTTACCCCATCTATTTCACCTTGCTACTTGGAATCTTCTTCTCCATCTCCATCATCTATTTT |
| | GGGCAAGTCCCAAAACAAGATCTCTCATGAATGTGTTGGTGACTCCTCTTCTTCTATGTGGGT |
| | TTATGTCTTTCTGGGCAATTTGTTGAGAGGTTTGGGTGAAACTCCAATTCAACCATTGGGTAT |
| | TGCTTACTTGGATGATTTCGCTTCTGAAGATAATGCCGCTTTCTACATTGGTTGTGTTCAAACC |
| | GTTGCTATTATCGGTCCAATTTTCGGTTTCTTGTTGGGTTCTTTGTGTGCCAAGTTGTACGTTG |
| | ATATTGGTTTCGTTAACTTGGACCACATTACTATCACTCCAAAAGATCCACAATGGGTTGGTG |
| | CTTGGTGGCTAGGTTACTTAATTGCTGGTTTCTTATCTTTGTTGGCTGCCGTTCCATTTTGGTG |
| | TTTGCCAAAAACTTTGCCAAGATCTCAGTCCAGAGAAAACTCTGGTTCTACTTCTGAAAAGTC |
| | CAAGTTCATTGATGACCCAATCCATTATCAAATGGCTCCAGGTGATGATAAGATGAAGATTA |
| | TGGAAATGGCCAAGGACTTCCTGCCATCTTTGAAAACTTTGTTTAGAAACCCCGTCTACATCT |
| | TGTACTTGTGTGCTTCTACTGTGCAGTTCAATTCTTTGTTCGGTATGGTTACTTACAAGCCCAA |
| | GTACATCGAACAACAATACGGTCAATCATCCTCTAAGGCCAATTTCGTTATTGGCTTGATTAA |
| | CATTCCAGCTGTTGCCTTGGGTATTTTTAGTGGTGGTATAGTCATGAAGAAGTTCAGATTGG |
| | GTATCTGTGAAGCCACTAAGTTGTATTTGGGTTCCTCTGTTTTTGGCTACCTGTTGTTTTTGTC |
| | TTTGTTTGCTTTGGGTTGCGAGAACTCTTCTGTTGCAGGTTTGACTGTTTCTTACCAAGGTAC |
| | AAAACCAGTCTCTTATCACGAAAGAGCTTTGTTCTCTGATTGCAACTCTAGATGTAAGTGCTC |
| | TGATTCTAAATGGGAACCTATGTGTGGTGATAACGGTATCACTTATGTTTCTGCTTGTTTGGC |
| | TGGTTGCCAATCATCTTCTAGATCTGGTAAGAACATCATCTTCAGCAACTGTACCTGTGTTGG |
| | TTTTGCTGCTCCAAAATCTGGTAATTGGTCTGGTATGATGGGTAGATGTCAAAAGGATAACG |
| | GTTGCTCTCAAATGTTCCTGTACTTCTTGGTTATCTCTGTCATCACTTCTTACACCTTGTCTTTA |
| | GGTGGTATTCCAGGCTACATTTTGTTGTTGAGATGTATCCAACCACAGTTGAAGTCTTTCGCT |
| | TTAGGTATCTATACCTTGGCCGTTAGAGTTTTGGCAGGTATTCCTGCTCCAGTTTATTTCGGT |
| | GTTTTGATTGATACCTCTTGCTTGAAGTGGGGTTTTAAGAAATGTGGTTCTAGAGGTTCTTGC |
| | AGGTTGTATGATTCTCATGCTTTCAGACATATCTACTTGGGTTTGACTACTTTGTTGGGTACTG |
| | TCTCTGTTTTCTTGTCTATGGCTGTTTTGTTCGTCCTGAAGAAGAAGTACGTTTCTAAGCACTC |
| | TTCATTGATTACCACCAGGGAAAAGATTGGCATGTCCTCTAGTATTAAGAAAGAAACCTGTG |
| | CTGCTAGAGACAGAGGTTTACAACCTAAATATTGGCCAGGTAAAGAAACCCGTTTGTGA |
| SEQ ID | MDTSSKENAHLFHKNSAQPAGGPSFTVGYPSTEEARPCCGKLKVFLGALSFVYFAKALAEGYLKS |
| NO: 948 | TVTQIERRFEIPSSLVGIIDGSFEIGNLLVITFVSYFGAKLHRPKIIGAGCLVMGFGTMLIAVPQFFM |
| | EKYSYEKYERYSPSSNVTPSISPCYLESSSPSPSSILGKSQNKISHECVGDSSSSMWVYVFLGNLLRG |
| | LGETPIQPLGIAYLDDFASEDNAAFYIGCVQTVAIIGPIFGFLLGSLCAKLYVDIGFVNLDHITITPKD |
| | PQWVGAWWLGYLIAGFLSLLAAVPFWCLPKTLPRSQSRENSGSTSEKSKFIDDPIHYQMAPGDD |
| | KMKIMEMAKDFLPSLKTLFRNPVYILYLCASTVQFNSLFGMVTYKPKYIEQQYGQSSSKANFVIGL |
| | INIPAVALGIFSGGIVMKKFRLGICEATKLYLGSSVFGYLLFLSLFALGCENSSVAGLTVSYQGTKPVS |
| | YHERALFSDCNSRCKCSDSKWEPMCGDNGITYVSACLAGCQSSSRSGKNIIFSNCTCVGFAAPKS |
| | GNWSGMMGRCQKDNGCSQMFLYFLVISVITSYTLSLGGIPGYILLLRCIQPQLKSFALGIYTLAVR |
| | VLAGIPAPVYFGVLIDTSCLKWGFKKCGSRGSCRLYDSHAFRHIYLGLTTLLGTVSVFLSMAVLFVL |
| | KKKYVSKHSSLITTREKIGMSSSIKKETCAARDRGLQPKYWPGKETRL |
| SEQ ID | ATGGACCAAAACCAGCACTTGAACAAAACTGCTGAAGCTCAACCATCCGAAAACAAAAAGA |
| NO: 949 | CTAGATACTGCAACGGCCTGAAGATGTTTTTGGCTGCTTTGTCCTTGTCTTTCATTGCTAAAAC |
| | TTTGGGTGCCATCATCATGAAGTCCTCCATTATTCATATCGAGAGAAGGTTCGAGATCTCCTC |
| | TTCTTTGGTTGGTTTTATCGATGGTTCCTTCGAAATCGGTAACTTGTTGGTTATCGTGTTCGTT |
| | TCTTACTTCGGTTCCAAATTGCATAGGCCAAAGTTGATTGGTATTGGTTGCTTCATTATGGGT |
| | ATCGGTGGTGTTTTGACTGCTTTACCACATTTCTTTATGGGCTACTACAGGTACTCCAAAGAG |
| | ACTAATATCGACTCCTCTGAAAACTCTACTTCTACCTTGTCTACCTGCTTGATTAACCAGATCT |
| | TGTCTTTGAATAGAGCCTCTCCAGAAATCGTTGGTAAGGGTTGTTTGAAAGAATCCGGTTCTT |
| | ATATGTGGATCTACGTGTTTATGGGTAACATGTTGAGAGGTATTGGTGAAACTCCAATAGTT |
| | CCATTGGGTTTGTCCTACATTGATGACTTTGCTAAAGAAGGCCACTCCTCATTATACTTGGGT |
| | ATTTTGAATGCCATTGCCATGATCGGTCCAATTATTGGTTTTACCTTGGGCTCTTTGTTCTCCA |
| | AGATGTACGTTGATATTGGTTACGTCGATTTGTCCACCATTAGAATTACTCCAACTGATTCTA |
| | GATGGGTTGGTGCTTGGTGGTTGAATTTCTTGGTTTCTGGTTTGTTCAGCATCATCTCCTCTAT |
| | TCCTTTCTTCTTCTTGCCACAAACTCCAAACAAGCCACAAAAAGAAAGAAAGGCCTCTTTGTC |
| | ATTGCACGTCTTGGAAACTAATGACGAAAAGGATCAAACTGCCAACTTGACCAATCAAGGTA |
| | AGAACATTACTAAGAACGTCACCGGTTTCTTCCAGTCCTTTAAGTCTATTTTGACTAACCCCTT |
| | GTACGTCATGTTCGTTTTGTTGACTTTGTTGCAGGTCAGCTCTTACATTGGTGCTTTTACTTAC |
| | GTTTTCAAGTACGTCGAACAACAATACGGTCAACCATCTTCTAAGGCCAATATTTTGTTGGGT |
| | GTTATCACGATTCCAATCTTCGCTTCTGGTATGTTTTTAGGTGGTTACATTATCAAGAAGTTCA |
| | AGTTGAACACCGTTGGTATTGCTAAGTTCTCTTGTTTCACTGCCGTTATGTCACTGTCTTTCTA |
| | CTTGTTGTACTTCTTCATCTTGTGCGAGAACAAATCTGTTGCTGGTTTGACTATGACTTACGAT |
| | GGTAACAATCCAGTTACCTCTCATAGAGATGTTCCATTGTCTTACTGTAACTCCGATTGCAAC |
| | TGTGATGAATCTCAATGGGAACCAGTTTGTGGTAACAACGGTATTACTTACATTTCTCCATGT |
| | TTGGCCGGTTGCAAATCTTCTTCAGGTAACAAAAAGCCCATCGTGTTCTACAACTGTTCTTGC |
| | TTGGAAGTTACCGGTCTGCAAAACAGAAATTACTCTGCTCATTTGGGTGAATGCCCAAGAGA |
| | TGACGCTTGTACTAGAAAGTTTTACTTCTTCGTCGCCATCCAAGTCTTGAACTTGTTTTTCTCA |
| | GCACTAGGTGGTACTTCCCATGTTATGTTGATTGTCAAAATCGTCCAGCCAGAGTTGAAATCT |
| | TTGGCTTTGGGTTTTCACTCCATGGTTATTAGAGCCTTAGGTGGTATTTTGGCTCCAATCTATT |
| | TTGGTGCCTTGATTGATACCACCTGTATTAAGTGGTCTACCAACAACTGTGGTACTAGAGGTT |
| | CTTGTAGAACCTACAATTCTACCTCATTCTCCAGAGTCTACTTGGGTTTATCTTCTATGTTGAG |
| | GGTTTCCTCTTTGGTCTTGTACATCATTTTGATCTACGCCATGAAGAAAAAGTACCAAGAGAA |
| | GGATATTAACGCCTCCGAAAATGGTTCTGTTATGGATGAAGCTAACTTGGAGTCTCTGAACA |
| | AGAACAAACATTTCGTTCCATCTGCTGGTGCTGATTCTGAAACTCACTGTTAA |
| SEQ ID | MDQNQHLNKTAEAQPSENKKTRYCNGLKMFLAALSLSFIAKTLGAIIMKSSIIHIERRFEISSSLVGF |
| NO: 950 | IDGSFEIGNLLVIVFVSYFGSKLHRPKLIGIGCFIMGIGGVLTALPHFFMGYYRYSKETNIDSSENSTS |
| | TLSTCLINQILSLNRASPEIVGKGCLKESGSYMWIYVFMGNMLRGIGETPIVPLGLSYIDDFAKEGH |
| | SSLYLGILNAIAMIGPIIGFTLGSLFSKMYVDIGYVDLSTIRITPTDSRWVGAWWLNFLVSGLFSIISS |
| | IPFFFLPQTPNKPQKERKASLSLHVLETNDEKDQTANLTNQGKNITKNVTGFFQSFKSILTNPLYV |
| | MFVLLTLLQVSSYIGAFTYVFKYVEQQYGQPSSKANILLGVITIPIFASGMFLGGYIIKKFKLNTVGIA |
| | KFSCFTAVMSLSFYLLYFFILCENKSVAGLTMTYDGNNPVTSHRDVPLSYCNSDCNCDESQWEPV |
| | CGNNGITYISPCLAGCKSSSGNKKPIVFYNCSCLEVTGLQNRNYSAHLGECPRDDACTRKFYFFVAI |
| | QVLNLFFSALGGTSHVMLIVKIVQPELKSLALGFHSMVIRALGGILAPIYFGALIDTTCIKWSTNNC |
| | GTRGSCRTYNSTSFSRVYLGLSSMLRVSSLVLYIILIYAMKKKYQEKDINASENGSVMDEANLESLN |
| | KNKHFVPSAGADSETHC |
| SEQ ID | ATGTTGGAGAAGTTCTGCAACTCTACCTTCTGGAATTCTTCATTCTTGGATTCACCTGAAGCT |
| NO: 951 | GATTTGCCATTGTGTTTCGAACAAACTGTTTTGGTTTGGATCCCATTGGGTTTCTTGTGGTTGT |
| | TGGCTCCATGGCAATTATTGCATGTTTACAAGTCCAGGACCAAGAGATCTTCTACTACTAAGT |
| | TGTACTTGGCTAAGCAAGTTTTCGTTGGCTTCTTGTTGATTTTGGCTGCTATTGAATTGGCCTT |
| | GGTTTTGACTGAAGATTCTGGTCAAGCTACTGTTCCAGCTGTTAGATATACAAACCCATCCTT |
| | GTACTTAGGCACCTGGTTGTTAGTTTTGTTGATCCAATATTCCAGACAATGGTGCGTCCAAAA |
| | GAACTCTTGGTTTTTGTCTTTGTTCTGGATCCTGTCTATTTTGTGCGGTACTTTCCAATTCCAA |
| | ACCTTGATTAGAACCTTGTTGCAAGGTGACAATTCCAACTTGGCTTACTCTTGCTTGTTCTTCA |
| | TTTCCTACGGTTTCCAGATCCTGATCTTGATTTTCTCTGCTTTCTCCGAGAACAACGAGTCATC |
| | TAACAATCCATCTTCCATTGCCTCATTCTTGTCCTCTATTACCTATTCCTGGTACGACTCCATTA |
| | TCTTGAAGGGTTACAAAAGACCATTGACCTTGGAAGATGTTTGGGAAGTTGATGAAGAGAT |
| | GAAGACTAAGACCCTGGTTTCTAAGTTCGAAACCCATATGAAGAGAGAATTGCAAAAAGCTA |
| | GAAGGGCATTGCAAAGAAGGCAAGAAAAATCCTCTCAGCAAAATTCTGGTGCTAGATTGCC |
| | AGGTTTGAACAAGAATCAATCCCAATCTCAAGATGCCCTGGTTTTAGAAGATGTCGAAAAGA |
| | AAAAGAAGAAGTCCGGTACTAAGAAGGATGTTCCAAAATCCTGGTTAATGAAGGCTTTGTTC |
| | AAGACCTTCTACATGGTCTTGTTGAAGTCCTTCCTGTTGAAATTGGTCAACGATATCTTCACCT |
| | TTGTCTCTCCACAACTGTTGAAGTTGTTGATCTCTTTCGCTTCTGATAGAGATACCTACTTGTG |
| | GATTGGTTACTTGTGCGCTATTTTGTTGTTTACCGCTGCCTTGATTCAATCCTTTTGCTTGCAA |
| | TGTTACTTCCAGTTGTGTTTCAAGTTGGGTGTTAAGGTTAGAACCGCTATTATGGCTTCCGTT |
| | TACAAAAAGGCTTTGACCTTGTCTAACTTGGCCAGAAAAGAGTATACAGTTGGCGAAACTGT |
| | TAACTTGATGTCCGTTGATGCTCAAAAGTTGATGGATGTTACCAACTTCATGCACATGTTGTG |
| | GTCCTCTGTTTTACAAATCGTCCTGTCGATATTCTTCTTGTGGCGTGAATTGGGTCCATCAGTT |
| | TTGGCTGGTGTTGGTGTTATGGTTTTGGTTATTCCAATCAACGCCATCTTGTCTACTAAGTCCA |
| | AAACTATCCAAGTCAAGAACATGAAGAACAAAGATAAGAGGCTGAAGATCATGAACGAGAT |
| | CTTGTCTGGTATCAAGATCTTAAAGTACTTCGCTTGGGAACCATCCTTCAGAGATCAAGTTCA |
| | AAACCTGAGGAAGAAAGAGCTGAAAAACTTGTTGGCTTTCTCCCAATTGCAATGCGTTGTTA |
| | TCTTCGTTTTCCAATTGACCCCAGTCTTGGTTTCTGTTGTTACTTTCTCTGTTTACGTCTTGGTC |
| | GACTCCAACAACATTTTGGATGCACAAAAAGCTTTCACGTCCATCACCTTGTTCAATATTTTGA |
| | GGTTCCCCTTGTCTATGCTGCCAATGATGATTTCTTCTATGTTGCAAGCTTCTGTCTCCACTGA |
| | AAGACTGGAAAAGTACTTAGGTGGTGATGATTTGGATACCTCCGCTATTAGACATGATTGCA |
| | ATTTCGATAAGGCCATGCAATTTTCCGAAGCTTCTTTTACTTGGGAACATGATTCTGAAGCTA |
| | CCGTTAGAGATGTCAACTTGGATATTATGGCCGGTCAATTAGTTGCTGTTATTGGTCCAGTTG |
| | GTTCCGGTAAATCTTCTTTGATTTCAGCTATGTTGGGCGAGATGGAAAATGTTCATGGTCATA |
| | TTACCATCAAGGGTACTACTGCTTATGTCCCACAACAATCCTGGATTCAAAACGGTACTATCA |
| | AGGACAATATCTTGTTCGGTACTGAGTTCAACGAAAAGAGATACCAACAAGTCTTGGAAGCT |
| | TGTGCTTTGTTGCCAGATTTGGAAATGTTGCCTGGTGGTGACTTGGCAGAAATTGGTGAAAA |
| | AGGTATCAATTTGTCCGGTGGTCAAAAGCAAAGAATCTCTTTGGCTAGAGCTACCTACCAAA |
| | ATTTGGACATCTACTTGTTGGATGATCCATTGTCAGCTGTTGATGCCCATGTTGGTAAACACA |
| | TCTTTAACAAAGTTTTGGGTCCAAACGGTTTGTTGAAAGGTAAAACTAGGTTGTTGGTTACCC |
| | ACTCCATGCATTTCTTGCCACAAGTTGACGAAATCGTTGTTTTAGGTAACGGTACAATCGTCG |
| | AAAAGGGTTCTTATTCTGCTTTATTGGCTAAGAAGGGTGAATTCGCCAAGAACTTGAAAACC |
| | TTCTTGAGACATACTGGTCCAGAAGAAGAGGCTACAGTTCACGATGGTTCTGAAGAGGAAG |
| | ATGACGATTACGGTTTGATCTCATCCGTTGAAGAAATACCAGAAGATGCTGCTTCTATCACCA |
| | TGAGAAGAGAAAACTCATTCAGAAGGACCCTGTCTAGATCCTCTAGATCTAATGGTAGACAC |
| | TTGAAGTCTCTGAGGAACTCTTTGAAAACCAGAAACGTCAACTCCTTGAAAGAGGATGAAGA |
| | GTTGGTTAAGGGTCAGAAGTTAATCAAGAAAGAGTTCATCGAAACCGGCAAGGTTAAGTTC |
| | TCTATCTACTTGGAATACTTGCAAGCCATCGGTTTGTTCTCCATCTTTTTCATTATTCTGGCCTT |
| | CGTCATGAACTCCGTTGCTTTCATTGGTTCTAACTTGTGGTTATCTGCTTGGACCTCTGATTCC |
| | AAGATTTTCAACTCTACTGATTACCCAGCCTCTCAAAGGGATATGAGAGTTGGTGTTTACGGT |
| | GCTTTGGGTTTAGCTCAAGGTATTTTCGTTTTCATTGCCCATTTTTGGTCTGCCTTTGGTTTTGT |
| | TCATGCCTCTAATATCTTGCACAAGCAGTTGTTGAACAACATATTGAGAGCACCAATGAGGTT |
| | CTTTGATACAACTCCAACTGGTAGAATCGTCAATAGATTCGCTGGTGATATTTCCACTGTTGA |
| | TGATACTTTGCCACAGTCTTTGAGATCTTGGATCACTTGTTTCTTGGGTATCATCTCTACCTTG |
| | GTTATGATCTGTATGGCTACTCCAGTTTTCACCATTATCGTTATTCCATTGGGCATCATCTACG |
| | TGTCTGTTCAAATGTTCTACGTTTCCACCTCTAGACAGTTGAGAAGATTGGATTCTGTTACCA |
| | GATCTCCAATCTACTCCCATTTCTCTGAAACTGTATCTGGTTTGCCAGTTATTAGAGCCTTCGA |
| | ACATCAACAGAGATTCCTAAAGCACAACGAAGTTAGAATCGATACCAATCAGAAGTGCGTCT |
| | TTTCCTGGATTACTTCTAATAGATGGTTGGCCATCAGATTGGAGTTGGTTGGTAATTTGACTG |
| | TTTTCTTCTCCGCTTTGATGATGGTTATCTACAGAGATACTTTGTCTGGTGATACCGTTGGTTT |
| | CGTTTTGTCTAACGCTTTGAACATCACCCAAACTTTGAACTGGTTGGTTAGAATGACCTCCGA |
| | AATCGAAACTAACATCGTTGCCGTTGAAAGAATTACTGAGTACACCAAGGTTGAAAATGAAG |
| | CTCCATGGGTTACTGACAAAAGGCCACCACCAGATTGGCCATCTAAAGGTAAGATTCAGTTT |
| | AACAACTACCAGGTCAGATACAGACCAGAATTGGATTTGGTTTTAAGGGGTATTACCTGCGA |
| | TATTGGCTCTATGGAAAAGATAGGTGTTGTTGGTAGAACTGGTGCTGGTAAATCTAGTTTGA |
| | CTAACTGTTTGTTCAGAATCTTAGAAGCTGCTGGTGGTCAGATTATTATCGATGGTGTTGATA |
| | TTGCCTCCATCGGTCTACATGATTTGAGAGAAAAGTTGACCATCATTCCACAAGACCCAATTT |
| | TGTTCTCTGGCTCTTTGAGAATGAACTTGGATCCCTTCAACAATTACTCCGACGAAGAAATTT |
| | GGAAGGCTTTAGAATTGGCCCACTTGAAATCTTTTGTCGCTTCTTTACAATTGGGCTTGTCAC |
| | ATGAAGGTACTGAAGCTGGTGGTAACTTATCTATTGGTCAAAGACAGTTGTTGTGCTTGGGT |
| | AGAGCTTTGTTGAGAAAGTCTAAGATTTTGGTCTTGGACGAAGCTACTGCTGCTGTTGATCT |
| | AGAAACCGATAATTTGATTCAAACCACCATCCAAAACGAATTCGCTCATTGCACTGTTATCAC |
| | CATTGCTCATAGATTGCATACCATCATGGATTCCGATAAGGTTATGGTGTTGGATAACGGTA |
| | AGATTATCGAATGTGGTTCTCCAGAAGAGTTGTTGCAAATTCCAGGTCCATTTTACTTCATGG |
| | CTAAAGAAGCTGGTATCGAGAACGTTAATTCTACCAAGTTCTAA |
| SEQ ID | MLEKFCNSTFWNSSFLDSPEADLPLCFEQTVLVWIPLGFLWLLAPWQLLHVYKSRTKRSSTTKLYL |
| NO: 952 | AKQVFVGFLLILAAIELALVLTEDSGQATVPAVRYTNPSLYLGTWLLVLLIQYSRQWCVQKNSWFL |
| | SLFWILSILCGTFQFQTLIRTLLQGDNSNLAYSCLFFISYGFQILILIFSAFSENNESSNNPSSIASFLS |
| | SITYSWYDSIILKGYKRPLTLEDVWEVDEEMKTKTLVSKFETHMKRELQKARRALQRRQEKSSQQN |
| | SGARLPGLNKNQSQSQDALVLEDVEKKKKKSGTKKDVPKSWLMKALFKTFYMVLLKSFLLKLVN |
| | DIFTFVSPQLLKLLISFASDRDTYLWIGYLCAILLFTAALIQSFCLQCYFQLCFKLGVKVRTAIMASVY |
| | KKALTLSNLARKEYTVGETVNLMSVDAQKLMDVTNFMHMLWSSVLQIVLSIFFLWRELGPSVLA |
| | GVGVMVLVIPINAILSTKSKTIQVKNMKNKDKRLKIMNEILSGIKILKYFAWEPSFRDQVQNLRKK |
| | ELKNLLAFSQLQCVVIFVFQLTPVLVSVVTFSVYVLVDSNNILDAQKAFTSITLFNILRFPLSMLPM |
| | MISSMLQASVSTERLEKYLGGDDLDTSAIRHDCNFDKAMQFSEASFTWEHDSEATVRDVNLDI |
| | MAGQLVAVIGPVGSGKSSLISAMLGEMENVHGHITIKGTTAYVPQQSWIQNGTIKDNILFGTEF |
| | NEKRYQQVLEACALLPDLEMLPGGDLAEIGEKGINLSGGQKQRISLARATYQNLDIYLLDDPLSAV |
| | DAHVGKHIFNKVLGPNGLLKGKTRLLVTHSMHFLPQVDEIVVLGNGTIVEKGSYSALLAKKGEFA |
| | KNLKTFLRHTGPEEEATVHDGSEEEDDDYGLISSVEEIPEDAASITMRRENSFRRTLSRSSRSNGRH |
| | LKSLRNSLKTRNVNSLKEDEELVKGQKLIKKEFIETGKVKFSIYLEYLQAIGLFSIFFIILAFVMNSVAFI |
| | GSNLWLSAWTSDSKIFNSTDYPASQRDMRVGVYGALGLAQGIFVFIAHFWSAFGFVHASNILHK |
| | QLLNNILRAPMRFFDTTPTGRIVNRFAGDISTVDDTLPQSLRSWITCFLGIISTLVMICMATPVFTII |
| | VIPLGIIYVSVQMFYVSTSRQLRRLDSVTRSPIYSHFSETVSGLPVIRAFEHQQRFLKHNEVRIDTNQ |
| | KCVFSWITSNRWLAIRLELVGNLTVFFSALMMVIYRDTLSGDTVGFVLSNALNITQTLNWLVRM |
| | TSEIETNIVAVERITEYTKVENEAPWVTDKRPPPDWPSKGKIQFNNYQVRYRPELDLVLRGITCDI |
| | GSMEKIGVVGRTGAGKSSLTNCLFRILEAAGGQIIIDGVDIASIGLHDLREKLTIIPQDPILFSGSLRM |
| | NLDPFNNYSDEEIWKALELAHLKSFVASLQLGLSHEGTEAGGNLSIGQRQLLCLGRALLRKSKILVL |
| | DEATAAVDLETDNLIQTTIQNEFAHCTVITIAHRLHTIMDSDKVMVLDNGKIIECGSPEELLQIPGP |
| | FYFMAKEAGIENVNSTKF |
| SEQ ID | ATGGATGCTTTGTGTGGTTCTGGTGAATTGGGTTCTAAATTCTGGGATTCCAACTTGTCTGTT |
| NO: 953 | CACACTGAAAATCCAGATTTGACCCCATGCTTCCAGAATTCTTTGTTGGCTTGGGTTCCATGT |
| | ATCTATTTGTGGGTTGCTTTGCCATGTTACTTGTTGTATTTGAGACATCACTGCAGGGGTTAC |
| | ATCATCTTGTCTCATTTGTCTAAGCTGAAGATGGTTTTGGGTGTTTTGTTGTGGTGTGTTTCTT |
| | GGGCTGATTTGTTCTACTCTTTCCATGGTTTGGTTCATGGTAGAGCACCAGCTCCAGTTTTCTT |
| | TGTTACTCCATTGGTTGTTGGTGTCACTATGTTGTTGGCTACCTTGTTGATTCAGTACGAAAG |
| | ATTGCAAGGTGTCCAATCTTCTGGTGTTTTGATCATTTTCTGGTTCTTGTGTGTTGTTTGCGCT |
| | ATCGTTCCATTCAGATCCAAGATTTTGTTGGCAAAAGCCGAAGGTGAAATCTCTGATCCTTTT |
| | AGATTCACCACCTTCTACATTCATTTCGCCTTGGTTTTGTCCGCTTTGATTTTGGCTTGTTTCAG |
| | AGAAAAGCCACCATTCTTCTCTGCCAAAAACGTTGATCCAAATCCATATCCAGAAACCTCTGC |
| | TGGTTTCTTGTCTAGATTATTCTTTTGGTGGTTCACCAAGATGGCTATCTATGGTTATAGACAC |
| | CCATTGGAAGAAAAGGACTTGTGGTCTTTGAAAGAAGAGGACAGATCTCAAATGGTTGTCC |
| | AACAATTATTGGAAGCTTGGAGGAAGCAAGAAAAGCAAACTGCTAGACATAAGGCTTCAGC |
| | TGCTCCAGGTAAAAATGCTTCAGGTGAAGATGAAGTTTTGTTAGGTGCTAGACCAAGACCAA |
| | GAAAGCCATCATTTTTGAAGGCTTTGTTAGCAACCTTCGGCAGCTCTTTCTTGATTTCTGCTTG |
| | CTTCAAGTTGATCCAGGACTTGTTGTCCTTTATTAACCCACAGTTGCTGTCCATCTTGATCAGA |
| | TTCATTTCTAATCCAATGGCTCCATCATGGTGGGGTTTCTTGGTTGCTGGTTTGGATGTTCCA |
| | GTTGCTCCTTGGATGCAATCCTTGATTTTACAACATTACTACCACTACATCTTCGTCACCGGTG |
| | TTAAGTTTAGAACAGGTATTATGGGTGTCATCTACAGAAAGGCTTTGGTTATTACCAACTCCG |
| | TTAAGAGGGCTTCTACTGTTGGTGAAATCGTTAACTTGATGTCCGTTGATGCTCAAAGATTCA |
| | TGGACTTGGCTCCATTCTTGAACTTGTTATGGTCTGCTCCATTGCAAATTATCCTGGCCATATA |
| | CTTTTTGTGGCAGAATTTGGGTCCATCTGTTTTGGCTGGTGTTGCTTTTATGGTTTTGTTGATC |
| | CCATTGAATGGTGCTGTTGCTGTTAAGATGAGAGCTTTCCAAGTTAAGCAGATGAAGCTGAA |
| | GGATTCCAGGATTAAGTTGATGTCTGAAATCTTGAACGGCATCAAGGTCTTGAAATTATACG |
| | CTTGGGAACCCAGCTTTTTGAAACAAGTTGAAGGTATCAGACAGGGTGAATTGCAACTATTG |
| | AGAACTGCTGCTTACTTGCATACCACTACTACTTTTACTTGGATGTGCTCCCCATTCTTGGTTA |
| | CTTTGATTACATTGTGGGTTTACGTTTACGTCGATCCAAACAATGTTTTGGATGCTGAAAAGG |
| | CCTTCGTTTCTGTCTCTTTGTTCAACATTTTGAGGCTGCCATTGAACATGTTGCCACAATTGAT |
| | TTCTAACTTGACCCAAGCCTCTGTGTCCTTGAAAAGAATTCAACAGTTTCTGTCCCAAGAAGA |
| | ATTGGATCCACAATCCGTTGAAAGAAAGACTATTTCTCCAGGTTACGCCATTACCATTCATTC |
| | TGGTACTTTTACATGGGCCCAAGATTTGCCACCAACTTTACATTCTTTGGACATTCAAGTTCCA |
| | AAGGGTGCTTTAGTTGCTGTTGTTGGTCCAGTTGGTTGTGGTAAATCTTCTTTGGTTTCTGCT |
| | TTGTTGGGTGAGATGGAAAAATTGGAAGGTAAGGTTCACATGAAGGGTTCTGTTGCTTATGT |
| | TCCACAACAAGCTTGGATTCAAAACTGTACCTTGCAAGAAAATGTCTTGTTCGGTAAAGCTTT |
| | GAACCCAAAGAGATACCAACAAACTTTAGAAGCTTGTGCCTTGTTGGCAGATTTGGAAATGT |
| | TGCCAGGTGGTGATCAAACTGAAATTGGTGAAAAGGGTATCAACTTGTCAGGTGGTCAAAG |
| | ACAAAGAGTTTCTTTGGCTAGAGCTGTTTATTCCGATGCCGATATTTTCTTGTTGGATGATCC |
| | ATTGTCTGCCGTTGATTCTCATGTTGCTAAGCACATTTTCGATCATGTTATTGGTCCTGAAGGT |
| | GTTTTGGCAGGTAAAACTAGAGTTTTGGTTACCCACGGTATTTCTTTCTTGCCTCAAACCGAT |
| | TTCATTATCGTTTTGGCCGATGGTCAAGTTTCTGAAATGGGTCCATATCCTGCTCTGTTGCAA |
| | AGAAATGGTTCTTTCGCTAACTTCTTGTGTAACTACGCTCCAGATGAAGATCAAGGTCATTTG |
| | GAAGATTCTTGGACAGCTTTGGAAGGTGCTGAGGACAAAGAAGCTTTGTTGATTGAAGATA |
| | CCTTGTCCAACCATACCGATTTGACTGATAATGATCCAGTTACCTACGTTGTCCAAAAGCAAT |
| | TCATGAGACAGTTGTCAGCTTTGTCATCTGATGGTGAAGGTCAAGGTAGACCAGTTCCAAGA |
| | AGGCACTTAGGTCCATCAGAAAAAGTTCAAGTTACTGAAGCTAAAGCTGATGGTGCTTTGAC |
| | TCAAGAAGAAAAAGCTGCTATTGGTACTGTCGAGTTGTCTGTCTTTTGGGATTATGCTAAAG |
| | CAGTTGGTTTGTGTACTACCTTGGCTATTTGTTTGTTGTACGTTGGTCAATCTGCTGCTGCAAT |
| | TGGTGCTAATGTTTGGTTGTCTGCATGGACTAATGATGCTATGGCTGATTCTAGACAAAACA |
| | ACACCTCTTTGAGATTAGGTGTTTATGCTGCTTTGGGTATCCTACAAGGTTTCTTAGTTATGTT |
| | GGCTGCTATGGCAATGGCTGCTGGTGGTATTCAAGCTGCAAGAGTTTTACATCAAGCCCTGT |
| | TGCATAACAAGATCAGATCACCACAATCTTTCTTCGATACAACTCCATCCGGTAGAATTTTGA |
| | ACTGCTTCTCTAAGGATATCTACGTCGTTGATGAAGTATTGGCCCCAGTTATTTTGATGCTGT |
| | TGAACTCCTTTTTCAACGCCATTTCTACCTTGGTTGTTATCATGGCTTCTACCCCTTTGTTCACC |
| | GTTGTTATTTTGCCATTGGCTGTCTTGTACACTTTGGTCCAAAGATTTTACGCTGCTACCTCTA |
| | GACAATTGAAGAGATTGGAATCCGTTTCCAGATCTCCAATCTACTCCCATTTTTCTGAAACTG |
| | TTACTGGTGCCTCTGTTATTAGAGCTTACAACAGATCTAGAGACTTCGAGATTATCTCCGATA |
| | CCAAGGTTGATGCCAATCAAAGAAGTTGCTACCCCTACATTATTTCCAACAGATGGTTGTCTA |
| | TTGGTGTCGAATTCGTTGGTAATTGCGTTGTTTTATTTGCTGCCTTGTTTGCCGTTATCGGTAG |
| | ATCTTCATTGAACCCAGGTTTGGTTGGTTTGAGTGTTTCTTACTCATTGCAAGTTACCTTCGCT |
| | TTGAACTGGATGATCAGAATGATGTCCGATTTGGAATCTAACATCGTTGCAGTCGAAAGGGT |
| | CAAAGAATACTCTAAGACTGAAACTGAAGCTCCATGGGTTGTCGAAGGTTCTAGACCTCCAG |
| | AAGGTTGGCCACCAAGAGGTGAAGTTGAATTCAGAAATTACTCCGTCAGATACAGACCAGG |
| | TTTAGATTTGGTTTTGAGGGATTTGTCCTTGCATGTTCATGGTGGTGAAAAAGTTGGTATCGT |
| | TGGTAGAACTGGTGCTGGTAAATCATCTATGACTTTGTGCTTGTTCAGAATCTTGGAAGCTGC |
| | TAAGGGTGAAATTAGAATCGATGGTTTGAACGTTGCTGATATCGGTTTACATGACTTGAGAT |
| | CCCAATTGACCATCATTCCACAAGATCCAATTTTGTTCTCCGGTACTTTGAGAATGAACTTGG |
| | ATCCTTTTGGTTCCTACTCCGAAGAGGATATTTGGTGGGCTTTAGAATTGTCTCACTTGCACA |
| | CTTTCGTTTCTTCACAACCAGCAGGTTTGGACTTCCAATGTTCTGAAGGTGGCGAAAATTTGT |
| | CTGTTGGTCAGAGACAATTGGTTTGCTTAGCTAGAGCTTTGCTGAGAAAGTCCAGAATATTG |
| | GTTTTAGATGAAGCCACTGCCGCCATTGACTTGGAAACTGATAATTTGATTCAGGCCACTATC |
| | AGAACTCAATTCGATACTTGTACCGTCTTGACCATTGCTCATAGATTGAACACCATTATGGAT |
| | TACACCCGTGTTCTGGTTTTGGATAAGGGTGTTGTTGCTGAATTCGATTCTCCAGCAAATTTG |
| | ATTGCTGCCAGAGGTATTTTCTATGGTATGGCTAGAGATGCTGGTCTGGCTTAA |
| SEQ ID | MDALCGSGELGSKFWDSNLSVHTENPDLTPCFQNSLLAWVPCIYLWVALPCYLLYLRHHCRGYII |
| NO: 954 | LSHLSKLKMVLGVLLWCVSWADLFYSFHGLVHGRAPAPVFFVTPLVVGVTMLLATLLIQYERLQG |
| | VQSSGVLIIFWFLCVVCAIVPFRSKILLAKAEGEISDPFRFTTFYIHFALVLSALILACFREKPPFFSAK |
| | NVDPNPYPETSAGFLSRLFFWWFTKMAIYGYRHPLEEKDLWSLKEEDRSQMVVQQLLEAWRK |
| | QEKQTARHKASAAPGKNASGEDEVLLGARPRPRKPSFLKALLATFGSSFLISACFKLIQDLLSFINP |
| | QLLSILIRFISNPMAPSWWGFLVAGLDVPVAPWMQSLILQHYYHYIFVTGVKFRTGIMGVIYRKA |
| | LVITNSVKRASTVGEIVNLMSVDAQRFMDLAPFLNLLWSAPLQIILAIYFLWQNLGPSVLAGVAF |
| | MVLLIPLNGAVAVKMRAFQVKQMKLKDSRIKLMSEILNGIKVLKLYAWEPSFLKQVEGIRQGELQ |
| | LLRTAAYLHTTTTFTWMCSPFLVTLITLWVYVYVDPNNVLDAEKAFVSVSLFNILRLPLNMLPQLIS |
| | NLTQASVSLKRIQQFLSQEELDPQSVERKTISPGYAITIHSGTFTWAQDLPPTLHSLDIQVPKGALV |
| | AVVGPVGCGKSSLVSALLGEMEKLEGKVHMKGSVAYVPQQAWIQNCTLQENVLFGKALNPKRY |
| | QQTLEACALLADLEMLPGGDQTEIGEKGINLSGGQRQRVSLARAVYSDADIFLLDDPLSAVDSHV |
| | AKHIFDHVIGPEGVLAGKTRVLVTHGISFLPQTDFIIVLADGQVSEMGPYPALLQRNGSFANFLCN |
| | YAPDEDQGHLEDSWTALEGAEDKEALLIEDTLSNHTDLTDNDPVTYVVQKQFMRQLSALSSDGE |
| | GQGRPVPRRHLGPSEKVQVTEAKADGALTQEEKAAIGTVELSVFWDYAKAVGLCTTLAICLLYVG |
| | QSAAAIGANVWLSAWTNDAMADSRQNNTSLRLGVYAALGILQGFLVMLAAMAMAAGGIQA |
| | ARVLHQALLHNKIRSPQSFFDTTPSGRILNCFSKDIYVVDEVLAPVILMLLNSFFNAISTLVVIMAST |
| | PLFTVVILPLAVLYTLVQRFYAATSRQLKRLESVSRSPIYSHFSETVTGASVIRAYNRSRDFEIISDTKV |
| | DANQRSCYPYIISNRWLSIGVEFVGNCVVLFAALFAVIGRSSLNPGLVGLSVSYSLQVTFALNWMI |
| | RMMSDLESNIVAVERVKEYSKTETEAPWVVEGSRPPEGWPPRGEVEFRNYSVRYRPGLDLVLRD |
| | LSLHVHGGEKVGIVGRTGAGKSSMTLCLFRILEAAKGEIRIDGLNVADIGLHDLRSQLTIIPQDPILF |
| | SGTLRMNLDPFGSYSEEDIWWALELSHLHTFVSSQPAGLDFQCSEGGENLSVGQRQLVCLARAL |
| | LRKSRILVLDEATAAIDLETDNLIQATIRTQFDTCTVLTIAHRLNTIMDYTRVLVLDKGVVAEFDSPA |
| | NLIAARGIFYGMARDAGLA |
| SEQ ID | ATGGGCTCCTCATTGGAAGATCAAAAGATCTTGCATAGACAGAAGAGGTTGTTGTC |
| NO: 955 | TCCATTCTTGTCTAAAAAGGTTCCACATGTTCCAACCGAAGATGAACGTAGACCATA |
| | TCCAGCTTCTAAGGCTAACGTTATCTCCAGAATTTTCTTCTGGTGGTTGTCCCCAGTT |
| | ATGAAAACTGGTTACAAAAGAACCTTGCAACCAGAGGATTTGTTCTACTTGACCGAT |
| | GATATTAAGGTTCAAACCATGGCCGATTCCTTCTACTTGTATATGACTCACGATATC |
| | GATAGAGCCAAGCAGAAGTTCTTGATCAAAAAGTGCAAAGAAAGGGGTGAAACCT |
| | TGGAAACTTCTTCCGTTGATAGAGAAGTTGACTTGAAGGACTTCCAGTTGTCTAAGT |
| | TCATTACCGTTTGGGCTTTAGCTAAGACTTTCAAGTGGCAATATTCTTGGGCTTGTTT |
| | CTGTTTGGCTTTGTCTAATGTTGGTCAAACTACCATGCCTTTGCTGACCAAAAAGTTG |
| | ATCCAATACGTTGAATTGAAGGCCTTGGGTGACGAAACTGGTATTGGTAAAGGTTT |
| | GGGTTACTCTTTTGGTACTGCTGCTATCGTTTTTATCGTTGGCGTTTTGATTAACCAC |
| | TTCTTCTACAGATCTATGTTGACTGGTGCTCAAGCTAAAGCTGTTTTGACAAAAGCTT |
| | TGCTGGACAAGTCTTTCAAGTTGTCCTCTGAAGCTAAACATAAGTACCCACCAGGTA |
| | AGATTACTTCCATGTTGGGTACTGATTTGTCCAGAATTGATTTCGCTTTGGGTTTCCA |
| | ACCATTCTTGATCGTTTTCCCAATTCCAATTGGTATCGCCATTGCCATTTTGATTGTCA |
| | ATATCGGTGTTTCCTCTTTGGTTGGTGTTGCTATTTTGTTCGTTTTCATGATCGGTATT |
| | GCTTTCTCTACTGGTGCTTTGTTGGCTTACAGAAAGAAGGCTAATTTCTTCACCGATA |
| | CCAGAGTCAACTACATCAAAGAAGCTCTGAACAACCTGAAGGTCATCAAGTTTTACT |
| | CTTGGGAACCACCATACCACGAAAACATTTCCAACGTCAGAAAGAAAGAGATGAGG |
| | ATCATCTACAGAATGCAGATCTTGAGAAATATCGTTACCTCCTTCGCTATGTCTTTGA |
| | CTTTGTTTGCTTCTATGACTGCCTTCTTGGTCTTGTACGCTATTGCTAATGGTAGAAG |
| | AGATCCAGCCTCTATCTTTTCCTCATTGTCTTTGTTCAACACTTTGACCCAACAGGTTT |
| | TCTTGTTGCCAATGGCTTTGGCTACAGGTGCTGATGCTTTTATGGGTATTTCTAGAG |
| | TTGGTGAGTTCATGTCCCAAAACGAAGTTCATCCTGAAGAAAACAGAATTGAAGCT |
| | CCACCTGAAGTCCAAAAAGAAATGGACAGAGAAGGTTTGGCCATCGAAGTTAATCA |
| | TGCTGATTTTGAGTGGGAAGTGTTCGAAGATGACGAAGAAGAAGAGGAAGCTGGT |
| | AAAGAAAAAGAGAAAGAAAAAGAAAAGAAGAAAGAGAAAGAGAACGGTTTCGAA |
| | GCTGGTTTCGAAAACAAAAAGAAAATGAACAAGATGTCCAAGAAAAAGCGTGAAA |
| | AGAAAGAAACTAAGAAGGCCGAATCCTTCGAAAACGAAACCATTCATGATTCCCAT |
| | ACCGAAAAGTTGTCTACCAAGGATTCTGATTTTACCGCTGATACCTCTAGTGAAGAG |
| | GAATTGGTTTTCGACGGTTTGAAGAACATCAACTTCAACATCAAGAAGGGTGAATT |
| | CGTTGTTATCACCGGTTTGATTGGTTCCGGTAAATCTTCTTTGTTAGCTGCTATGTCT |
| | GGTTTCATGAAGAGATTGAGAGGTTCTGTTAATGTCAACGGCTCTTTGTTGTTGTGT |
| | GGTCAACCATGGGTTCAAAACAACACTGTTAAGGACAACATTATCTTCGGCTCCAAA |
| | TACGACGAAGAAAAGTACAAGAGAGTGATCTACTCTTGCTCCTTGGAATCCGATTT |
| | GGAAATTTTGCCAGCTGGTGATAGAACCGAAATTGGTGAAAGAGGTATTACTTTGT |
| | CTGGTGGTCAAAAGGCTAGAATCAATTTGGCTAGAGCTGTTTATGCCGATAAGGAC |
| | ATCATTTTGTTGGATGATGTTTTGTCCGCTGTTGATGCAAGAGTTGGTATTCATATTA |
| | TGAACAACTGCATCCTGGACCTGCTTAAAGAGAAAACTAGAATCTTGGCTACCCATC |
| | AGTTGTCTTTAATTGGTTCTGCCGATCGTATCATCTTCTTGAATGGTGATGGTAGTGT |
| | TGATGTCGGTACTTTCGAAGAATTGAAAGAAGCTAACCCAGGTTTCGGTAACTTAAT |
| | GACTTACAACCACGAACACGAGAAAGAGGAAGAAGTCGATATAGAACAGTTGTCC |
| | GAAGAAGAGTTGGAAGAAGAGAAGAAGTTGATCGAAAGACAATTGACTCAGAGA |
| | ACTACCAGAACCACAAGATCTATTGGTCCTGAAAATGAGGAAGAGGACGACGACG |
| | AAGAGGCTAGACATCATGAATTCAATTTGGACGAAACAGCCGATGGTACTTTGATA |
| | GGTTCTGAAGAAAGGGCTGTTAATGCTATTGGTTGGGATGTTTACGCTAGGTACAT |
| | TAGATTAGGTTCAGGTAGATTCACTCCCTGGTTGATTTTGCCTGCTTTATTGACTGCT |
| | ATGCTGTTGTCTACTTTCTCCCAAATTTTCACTAACACCTGGTTGTCATTTTGGACCTC |
| | TTACAAGTTTAACAGGCCCAACAAATTCTACATCGGCATCTACATTATGTTCACGTTC |
| | TTGTCGTTCTTTCTGCTGACCATCGAATTCATCATCTTGGTCTATATTACCAACACCG |
| | CCTCTGTTATGTTGAATGTTATGGCTGTTAAGAGAGTTTTACACGCCCCAATGTCTTT |
| | TATGGATACAACTCCAATGGGCAGAATCTTGAACAGATTCACTAAGGATACCGATG |
| | TCTTGGATAACGAAATCGGTGATCAAATGCGTTTCTTCTTGTTCACCTTCTGCAACAT |
| | CATTGGTGTCTTGATTTTGTGCATCATCTACTTGCCATGGTTCGCTATTGCAATTCCA |
| | TTTTTGGGTGTCGTCTTTGTTTCCATTGCTAACTATTATCAAGCCTCCGCAAGAGAAG |
| | TTAAGAGATTGGAAGCTGTTCAAAGGTCCTTGGTTTACAACAATTTCAACGAAACCT |
| | TGTCTGGTATGCCAACCATTAAGGCTTATAATGCTACCTCTAGATTCATGGACAAGA |
| | ACAATTTCTTGATTGACAAGATGAATGAGGCCTACTACATCACTATCGCTAATCAAA |
| | GATGGTTGGCCATTCACATGGATTTCATTGCATCTTTGTTCGCCTTGTTGATTGCCTT |
| | GTTATGTGTCAACAGGGTGTTCAATATTGGTGCTTCTTCAGTTGGTCTGATCGTGTC |
| | TTACGTTTTTCAAATTGCTGGCCAGTTGTCCATGTTGATTAGAACTTTTACCCAAGTC |
| | GAGAACGAAATGAACTCCGCTGAAAGATTATCTTCTTACGCTACTTCTTTGCCTGAA |
| | GAGGCTCCATATGTTATTACTGAAAGAACTCCACCACCAGATTGGCCATCTAAAGGT |
| | GGTATTATCTTTGATCATGCTTCCTTGGCTTATAGACCAGGTTTGCCATTGGTTTTAA |
| | AGGACTTGACTTTCAGAGTTGAGCCCATGGAAAAGATTGGTATTTGTGGTAGAACT |
| | GGTGCTGGTAAGTCATCTATTATGACAGCCTTGTACAGGTTGTCTGAATTGGAATCT |
| | GGTAAGATCGAAATCGATGACATTGACATTTCTACCTTGGGTTTGAGAGACTTGAG |
| | ATCCAAATTGTCCATCATTCCTCAAGATCCAGTCTTGTTTAGAGGCACCATTAGAAA |
| | GAACTTGGATCCATTTGGTGAACACTCCGATGATAAGTTGTGGGATGCTTTGAGAA |
| | GAACTGGTTTGATCGAAGAATCCAGATTAGAAGCCGTCAAAAAGCAGACCAAAGTT |
| | TCTTCTGCTACTACTACAACAGCTGCCAATTCTGAAAAAGGTGATGCTACTGCTACT |
| | GCCACTACAACTACTCCATTGAACAAGTTCCATTTGGACCAAACCGTTGAAGATGAA |
| | GGTTCTAATTTCAGCTTGGGTGAAAGACAGTTGATTGCTTTTGCTAGAGCTTTGGTT |
| | AGAGACTCGAAAATCTTGATCTTGGATGAAGCCACATCCTCCGTTGATTACGAAACT |
| | GATTTCAAGGTTCAACAGACCATCGTTAGAGAATTTGGTGATTGCACCATTTTGTGT |
| | ATCGCCCATAGATTGAAAACCATCATCAACTACGACAGGATCTTGGTTTTGGATAAG |
| | GGTGAAATCAAAGAATTCGACACCCCACTGAACCTGTTTAATTTGGAAGGTGGCAT |
| | TTTCCAACAAATGTGCCAAAGATCTAACATCACCCAAGAGGATTTCTCTAACACCAA |
| | GAATTTCTAA |
| SEQ ID | MGSSLEDQKILHRQKRLLSPFLSKKVPHVPTEDERRPYPASKANVISRIFFWWLSPVMK |
| NO: 956 | TGYKRTLQPEDLFYLTDDIKVQTMADSFYLYMTHDIDRAKQKFLIKKCKERGETLETSSV |
| | DREVDLKDFQLSKFITVWALAKTFKWQYSWACFCLALSNVGQTTMPLLTKKLIQYVELK |
| | ALGDETGIGKGLGYSFGTAAIVFIVGVLINHFFYRSMLTGAQAKAVLTKALLDKSFKLSSE |
| | AKHKYPPGKITSMLGTDLSRIDFALGFQPFLIVFPIPIGIAIAILIVNIGVSSLVGVAILFVFMI |
| | GIAFSTGALLAYRKKANFFTDTRVNYIKEALNNLKVIKFYSWEPPYHENISNVRKKEMRII |
| | YRMQILRNIVTSFAMSLTLFASMTAFLVLYAIANGRRDPASIFSSLSLFNTLTQQVFLLPM |
| | ALATGADAFMGISRVGEFMSQNEVHPEENRIEAPPEVQKEMDREGLAIEVNHADFEW |
| | EVFEDDEEEEEAGKEKEKEKEKKKEKENGFEAGFENKKKMNKMSKKKREKKETKKAESF |
| | ENETIHDSHTEKLSTKDSDFTADTSSEEELVFDGLKNINFNIKKGEFVVITGLIGSGKSSLLA |
| | AMSGFMKRLRGSVNVNGSLLLCGQPWVQNNTVKDNIIFGSKYDEEKYKRVIYSCSLES |
| | DLEILPAGDRTEIGERGITLSGGQKARINLARAVYADKDIILLDDVLSAVDARVGIHIMNN |
| | CILDLLKEKTRILATHQLSLIGSADRIIFLNGDGSVDVGTFEELKEANPGFGNLMTYNHEH |
| | EKEEEVDIEQLSEEELEEEKKLIERQLTQRTTRTTRSIGPENEEEDDDEEARHHEFNLDETA |
| | DGTLIGSEERAVNAIGWDVYARYIRLGSGRFTPWLILPALLTAMLLSTFSQIFTNTWLSF |
| | WTSYKFNRPNKFYIGIYIMFTFLSFFLLTIEFIILVYITNTASVMLNVMAVKRVLHAPMSF |
| | MDTTPMGRILNRFTKDTDVLDNEIGDQMRFFLFTFCNIIGVLILCIIYLPWFAIAIPFLGVV |
| | FVSIANYYQASAREVKRLEAVQRSLVYNNFNETLSGMPTIKAYNATSRFMDKNNFLIDK |
| | MNEAYYITIANQRWLAIHMDFIASLFALLIALLCVNRVFNIGASSVGLIVSYVFQIAGQLS |
| | MLIRTFTQVENEMNSAERLSSYATSLPEEAPYVITERTPPPDWPSKGGIIFDHASLAYRP |
| | GLPLVLKDLTFRVEPMEKIGICGRTGAGKSSIMTALYRLSELESGKIEIDDIDISTLGLRDLR |
| | SKLSIIPQDPVLFRGTIRKNLDPFGEHSDDKLWDALRRTGLIEESRLEAVKKQTKVSSATT |
| | TTAANSEKGDATATATTTTPLNKFHLDQTVEDEGSNFSLGERQLIAFARALVRDSKILILD |
| | EATSSVDYETDFKVQQTIVREFGDCTILCIAHRLKTIINYDRILVLDKGEIKEFDTPLNLFNL |
| | EGGIFQQMCQRSNITQEDFSNTKNF* |
| SEQ ID | ATGGCTGTTTACGCTAGAAGAACCCCAGTTATTACTGTTAGACACCAAAGAATCTTC |
| NO: 957 | GGCTTCAGATCCATTTCTGTTCAATTGACTGCTTTGGCTGTTGCCTTTAGATCTCAAG |
| | CTCATCAAAAGGTTGATTCCAACACCGTTATCTACCAAGCTTATAGAGTTCCACCATT |
| | GCCATCTCAAGACGAAAGAAATGCTTTTCCAGAAAAGACTGCTAACCCATTGTCCAG |
| | AGTTTTCTTTTGGTGGTTGAATCCAGTTATGAAGACCGGTTACAAAAGAACCTTGCA |
| | ACCAGATGATTTGTTCTACTTGGGTGATGATTTCGTTGTTAAGCCAAAGGCCGATAA |
| | GTTCATCGAAATCTTCAACAGAAGATTGGCCAAGGCCAAAGAAAACCATATCTTGC |
| | AAAAGTGCAAAGAGAGGAACGAATCTATCGATTCCTCATCCGTTTCCAAAGAAGAG |
| | GATATGGAAGATTTTTCACCACCAAAGGCTATTACCGCTTTGTCTTTGTTGGAAACC |
| | TTCAAGATCAGATACTTCATGGCTTGTTTGTTGTTGACCATCTGTAACGTTGCTCAAA |
| | CTTTGAATCCCCTGTTGACCAAGAAGTTGATCCAATTTGTTGAAAGACGTGCTGCTG |
| | GTGTTGAATCTAGAGTTGGTCCAGGTGTTGGTTATGCTATTGGTGCTTCTTTGCTGG |
| | TTTTGGTTATTGGTATTTTCGTCAACCACTTCTTCTACAACTCTATGATGTGTGGTGC |
| | TTTCTCTAAAGCTGTTTTGACTAAGGCTATGCTGGACAAGTCTTTTAAGCAAAATGC |
| | CGAATCCAGATCTAAATACGGTGCTGGTAAAGTCACTTCTATGATGGGTACTGATTT |
| | GGCCAGAATTGATTTCGCTATTGGTTTCCAGCCATTCATCATTACTTTTCCAGTTCCA |
| | GTCATTATCGCTATCGCCATTTTGATCGTTAATGTTGGTCCATCTGCTTTGGTTGGTG |
| | TAGGTTTGTTACTGTTCTTCGTGTTTTTCATGATGTGGTGCGCTAAGCAATTCTTCTC |
| | ATACAGAGTTATTGCCAACAAGTTCACCGATAAGAGGGTGTCTTATATCAAAGAGG |
| | TCCTGAACAACCTGAAGATCATCAAGTTTTATTCTTGGGAACCACCATACCACGAAA |
| | ACATTTCTGATGTCAGGTACAAAGAGATGAAGAACATCTTGAAGATGCAGGTCTTG |
| | AGGAACATTTTGATGGCTTTGGCTATGTCTATGACCACCTTATCTTCTATGGCTACTT |
| | TCTTGACCTTGTACGGTGCTAAAAATGGTAAAGATGATGCCGCCTCTATCTTCTCCTC |
| | AATTTCTTTGTTTAACGTCTTGTCCCAGCAGGTTTTGATGTTGCCATTGGCTTTAGCA |
| | GCCGGTGTTGATTGCATGACTGGTTTGATAAGAGTCGGTAGATATTTGGCCTCCTCT |
| | GAAATTGATCCAGAATCCAATAGAATCGATGCCGACTCTGAAAAGATCGTTGAAAT |
| | GGATTCTAACGACTTGTCTATCGAAGTTAACAACGCTAACTTCGAATGGGAAACTTT |
| | CGAATTGCAGGACGAAGAAAACGAAGTTGACTTGATCAACCTGAACAAGAAGTAC |
| | AGAAAGCAATTGGCCAGAGAAGAGAAGCTTAAGAAGAAGTTGTCTCAGAACTCTTC |
| | TACCGAAAACGACGAAAAGTTGGAAGAATCTAACTACCATGCAGACCCAGAAGGT |
| | AAAGAATTGAAGGATTCTTCTTCCAATGACACCTCCTTGGAAGATTCAAATTTCGCT |
| | GGTTTGAACAACATCGACTTGAAGATTAAGAAGGGTGAATTCGTTGTCATCACCGG |
| | TTTGATTGGTTCTGGTAAATCCTCTTTGTTGTTAGCCTTGTCCGGTTTTTATGAAGAGA |
| | ACTTCTGGTTCTGTTAACGTCAACGGTTCCTTGTTGTTGTGTGGTTACCCATGGATTC |
| | AAAACGATACCGTTAAGTCCAACATCTTGTTCGGTGAAGAATTGGATGAAGCCAAG |
| | TACTCTGAAGTTGTTTACGCATGTTCATTGGAATCCGACTTGGAAATTTTGCCAGCT |
| | GGTGATGCTACTGAAATTGGTGAAAGAGGTATTACTTTGTCCGGTGGTCAAAAGGC |
| | TAGAATCAATTTGGCTAGAGCTGTCTACAACAACAAGGACATTATCTTGTTGGACGA |
| | TGTTTTGTCTGCTGTTGATGCAAGAGTTGGTAAGCACATTATGAACAACTGCATCTT |
| | GGGTTTGTTGAAGGATAAGACCAGAATTTTGGCTACCCACCAATTGTCTTTAATCGG |
| | TTCTGCTGATAGAGTCATCTTCTTGAATGGTGATGGTTCAGTTTCTGTTGGTGCTGTC |
| | GAAGAATTGAGGGAAAACAATTTCGCCTTTAACAACTTGATGGCCTTCAACTCTGAA |
| | ACTAAGGATGAAGAAGAAGAGAAAGAGGAAGAAAACGAGGACGAGGAACAAGA |
| | AGCTCAAGAATTCGAATCCATCAAGAGACAGTTGTCCAAGATTCCCACTAACAAAG |
| | AAAAAGATGAAGAGGCCATCCACAAGGATTACAATCAAAACAATACTGAGGACGG |
| | CAAGTTGATGGCATCTGAAGATAGGGCTATCAACGGTATTAAGTTGGATGTCTACA |
| | AAAAGTACATCCACTACGGTTCCGGTAAGATTGGTTCATTGGTTATGTGTTTTGCCA |
| | TCGTTTTGGCAGTTGTTTTGGCAACTTTCTGCCAATTATTCACTAACACCTGGTTGTC |
| | ATTCTGGACCGAAAAGAAATTTCCAGGTAAGTCTGATGGTTTCTACATCGGTTTTTA |
| | CGTTATGTTCGCTGTCTTGTCTGGCCTGTTGATTATGATTGAGTTTATCTTCTTCGTG |
| | CTGTTGACTAACACTGCCTCTAAGAACTTGAATATCTTGGCCGTTAAGAAAGTCTTG |
| | CATGCTCCAATGTCTTTCATGGATACAACTCCAATGGGTAGAATCTTGAACAGATTC |
| | ACTAAGGATACCGATGCCTTGGATAATGAATTGGGTGATCAAATCCGTATGCTGTTC |
| | TTCTTTTTCGGTAATATTGCCGGTGTTTTGATCTTGTGTGTTATCTACTTGCCATGGTT |
| | CGCAATTGCTATTCCATTTTTGGTTGCTTTGTTCATCGGTGTTGCTAATTACTATCAA |
| | GCTTCCGCTAGAGAAATCAAGAGATTGGAAGCTTTACAAAGGTCCCACGTTTACAA |
| | CAACTTTAACGAAACATTGAACGGCATGAACACCATTAAGGCTTACAAAGCTGATC |
| | ACATCTACTTGAGGAAGAACGAGTTTTTCCTAAACAAGATGAACGAAGCCTACTACT |
| | TGACTATTGGTAATCAAAGATGGCTGGCCATTCATTTGGATTGCTTGGCTACTATTT |
| | TCGCCTTGATTATTGCCTTGTTGTGCGTTTTTAGGGTGTTCTCTATTGGTCCAGCTTC |
| | AGTTGGTTTGTTATTGTCTTACGTCTTGCAAATTGCCGGTCAGTTGTCTATGTTGATT |
| | AGAACTTACACCCAGGTCGAAAACGAAATGAATGCAGTTGAAAGAATGTGCTCCTA |
| | CGCTTTTGATTTGCCTCAAGAAGCCCCATTCAAAATCTCTGAAACAACTCCACCACCA |
| | GAATGGCCCGAACAAGGTTCTATTAAGTTTGAAGATGTTTCCTTGGCTTACAGACCA |
| | GGTTTGCCATTAGTTTTGAAGAACTTGTCCTTGCAAGTTGAGCCATCACAAAAGATT |
| | GGTATCTGTGGTAGAACTGGTGCAGGTAAATCTTCTATTATGACTGCCTTGTACAGG |
| | CTGTCTGAATTGTCTACTGGTAAGATCGAAATCGATGGTATCGACATTTCGTCTTTG |
| | GGTTTGAGTTCTTTGAGGTCCAAATTGTCCATCATTCCACAAGATCCAGTTTTGTTCC |
| | AAGGCACCATTAGAAAGAATCTGGACCCTTTTAATGAACACCCTGATGACTTGTTGT |
| | GGGATTCTTTGCGTAGAGCTGGTTTAATTGAAGAGGCTAAATTGAAGTCCGTCAAG |
| | AGGCAAAATGATGAGTCTGAAGAATACCACAAGTTCCACTTGAATCAAGAGGTTGA |
| | AGATGATGGTGCTAACTTCTCATTGGGTGAAAGACAGTTGATTGCTTTTGCTAGAGC |
| | TTTGGTTAGAGACTCGAAGATCTTGATTTTAGACGAAGCTACTTCCTCCGTTGATTA |
| | CGAAACTGACAACAAGATTCAATCTACCATCGTCAGGGAATTCTCTAAGTGTACCAT |
| | TTTGTGCATTGCCCACAGATTGAAAACCATCTTGAACTACGATAAGATCCTGGTCTT |
| | GGATAAGGGTGAGATCAAAGAATTTGATACCCCATGGAATTTGTTCAACACCGAGG |
| | ATTCTATTTTCCAGCAAATGTGCGAAAGATCCAACATCACAGTCGAAGATTTCCAAA |
| | ACTTGCAGAGGATGTGA |
| SEQ ID | MAVYARRTPVITVRHQRIFGFRSISVQLTALAVAFRSQAHQKVDSNTVIYQAYRVPPLPS |
| NO: 958 | QDERNAFPEKTANPLSRVFFWWLNPVMKTGYKRTLQPDDLFYLGDDFVVKPKADKFIE |
| | IFNRRLAKAKENHILQKCKERNESIDSSSVSKEEDMEDFSPPKAITALSLLETFKIRYFMAC |
| | LLLTICNVAQTLNPLLTKKLIQFVERRAAGVESRVGPGVGYAIGASLLVLVIGIFVNHFFYN |
| | SMMCGAFSKAVLTKAMLDKSFKQNAESRSKYGAGKVTSMMGTDLARIDFAIGFQPFII |
| | TFPVPVIIAIAILIVNVGPSALVGVGLLLFFVFFMMWCAKQFFSYRVIANKFTDKRVSYIKE |
| | VLNNLKIIKFYSWEPPYHENISDVRYKEMKNILKMQVLRNILMALAMSMTTLSSMATFL |
| | TLYGAKNGKDDAASIFSSISLFNVLSQQVLMLPLALAAGVDCMTGLIRVGRYLASSEIDP |
| | ESNRIDADSEKIVEMDSNDLSIEVNNANFEWETFELQDEENEVDLINLNKKYRKQLARE |
| | EKLKKKLSQNSSTENDEKLEESNYHADPEGKELKDSSSNDTSLEDSNFAGLNNIDLKIKKG |
| | EFVVITGLIGSGKSSLLLALSGFMKRTSGSVNVNGSLLLCGYPWIQNDTVKSNILFGEELD |
| | EAKYSEVVYACSLESDLEILPAGDATEIGERGITLSGGQKARINLARAVYNNKDIILLDDVL |
| | SAVDARVGKHIMNNCILGLLKDKTRILATHQLSLIGSADRVIFLNGDGSVSVGAVEELRE |
| | NNFAFNNLMAFNSETKDEEEEKEEENEDEEQEAQEFESIKRQLSKIPTNKEKDEEAIHKD |
| | YNQNNTEDGKLMASEDRAINGIKLDVYKKYIHYGSGKIGSLVMCFAIVLAVVLATFCQLF |
| | TNTWLSFWTEKKFPGKSDGFYIGFYVMFAVLSGLLIMIEFIFFVLLTNTASKNLNILAVKK |
| | VLHAPMSFMDTTPMGRILNRFTKDTDALDNELGDQIRMLFFFFGNIAGVLILCVIYLPW |
| | FAIAIPFLVALFIGVANYYQASAREIKRLEALQRSHVYNNFNETLNGMNTIKAYKADHIYL |
| | RKNEFFLNKMNEAYYLTIGNQRWLAIHLDCLATIFALIIALLCVFRVFSIGPASVGLLLSYV |
| | LQIAGQLSMLIRTYTQVENEMNAVERMCSYAFDLPQEAPFKISETTPPPEWPEQGSIKF |
| | EDVSLAYRPGLPLVLKNLSLQVEPSQKIGICGRTGAGKSSIMTALYRLSELSTGKIEIDGIDI |
| | SSLGLSSLRSKLSIIPQDPVLFQGTIRKNLDPFNEHPDDLLWDSLRRAGLIEEAKLKSVKRQ |
| | NDESEEYHKFHLNQEVEDDGANFSLGERQLIAFARALVRDSKILILDEATSSVDYETDNKI |
| | QSTIVREFSKCTILCIAHRLKTILNYDKILVLDKGEIKEFDTPWNLENTEDSIFQQMCERSNI |
| | TVEDFQNLQRM* |
| SEQ ID | ATGGTCGAATTGGAAAAGGGTTCCGAACCAGAATTGCAACATAGATTATTGACCCC |
| NO: 959 | ATTCCTGTCTAAAAAGGTTCCACCAGTTCCACATGATGAAGATAGACCATATCATCC |
| | AAAATGGCGTAACCCATTCTCTTTCTTGTTTTTCACTTGGTTGACCCCAGTTTTGTTG |
| | GTTGGTTACAAAAGAACTTTGTTGCCAGAGGACATGTTCAAGTTGCATGAAGGTAT |
| | TACTGCTGAACATTTGGCCGAAAAGTTCCAAAGAATCTTCGACAGAAGATTGGCCC |
| | AAGATAAGCAAAGACACTTGAAAGAAAAAGCTAAGGCTAGAGGTGAAACCTTGGA |
| | AACTTCTTCTGTTGAATCCGTTGATGACATGGCTGATTACGAATTGTCTAAGTCTTTG |
| | TGCTTCTTGACCTTGTACGAAACTTTCGCTAGACAATACTCTTTGGCTTTGGTTTTTG |
| | CTACCTTGGGTATGTCTTGTTCTACCTGTATTCCTTTGTTGTCCCGTAAGCTGATTAA |
| | CTTCGTTTCTGAAAAAGCCTACGGCTTCAATTTGAATATGGGTACTGGTGTTGGTTA |
| | CGCTATTGGTGTTGCTATTTTGATCTTCACTGGTGACGTTTTGATTAACCAGGGTGTT |
| | TACTTGTCTATGATTACCGGTGCTCAAATTAGAGCTGTTTTCACCAAGTTGTTGCTG |
| | GACAAGTCTTTTAAGTTGAACGCCAAGTCTAGAAAGCAATTCCCAGCTTCTAAGATT |
| | ACCGCTATTATGTCTACCGATGTTTCCAGAGTTGACTTAGGTACTGGTTTTTCTATCT |
| | ACGGTTTCGTGTTTGTTTTCCCAGTTGGTATTTCCATTGGTATCTTGGTCTACAACAT |
| | TAAGGCTCCAGCTATGGTTGGTGTAGGTTTGATGATTGCCTTTTTGTTCGTTGCTGG |
| | TATTTTGGGTGCTATGTTGTTCTCTTTCAGAAAAACCGCTCAAAAGTCCACTGATGCT |
| | AGAGTTTCTTACATGAAGGAAGTCCTGAACAACCTGAAGATGATCAAGTTTTACTCT |
| | TGGGAAAAGCCCTACTTCAGCTTGATTTCCAAAATCAGACGTAGAGAAATGGCCTA |
| | CTTGTTGAGAATGGAAATCACGAGAATGATCATAATCACCTTGGCTTCATCTCTGAC |
| | CTTGATTTCTTCTTTGGCATCCTTCTTGACTCTGTATGCTATTGCTTCTCCATCTTCTA |
| | GAAATCCAGCCGATATTTTCTCATCCGTTGCTCTGTTTAATATGTTGGCTGGTCAATT |
| | CGTTGTCCTGCCATTGTCTATTGCTGGTTCTACTGATGCATTTTTGGGTATGAATAGA |
| | GTTGCTGCTGTTTTGGCTGCTGACGAAATTGATCCAAAAGATTCCGTTAGATTGATC |
| | ACCGATGACGAAAGAACTGCTATGCAAGAAAACAAGTTGGCTGTTTCTGTTAGAGA |
| | TTGCGATTTCGAATGGGAGATCTTCGATTTGAAAGAGGAAAAGACTGAGGACCAA |
| | ACGAAGGATAACAAAGAGCTGAAAAAAGAAAAGAAAGAAATGAAGAAGAAAAAG |
| | AAAGAAGAGAAGAAAGCCCAGAAGGCCTCTAAATCTAATTCTCCATCACCTGAAGT |
| | TGACGAAAAGACAGGTGAAGTTTCTAGCTTCAAGTTGAACAACATCAACTTGGATG |
| | TTAAGGACGGTGAATTCGTCGTTATTACAGGTTCTATTGGTTCCGGTAAGTCCTCTT |
| | TGTTGCATGCTTTGGATGGTACGATGAAGAAAAACTCTGGTAAGTTGTTGTTGAAC |
| | GGTTCCTTGTTGATGTGTGGTGTTCCATGGATTCAAAACAACACCTTGAGAGAGAA |
| | CATCTTGTTTGGTTCTCCATATGACGAAGCTTGGTACAACAAGGTTGTTGAAGCTTG |
| | TTCTTTGAACTCCGATTTTGATTTGTTGCCTGCTGGTGATAGAACCGAAATTGGTGA |
| | AAGAGGTATTACATTGTCTGGTGGTCAAAAAGCCAGAGTTTGTTTGGCTAGAACTG |
| | TCTACGAAGATTCCTCCATTATCTTGTTGGATGATGTTTTGTCAGCTGTTGATGCCAA |
| | GGTTGGTAAGCACATTATGAACGAATGTTTGTTGGGCCTGTTGAAGAACAAAACTA |
| | GAATTTTGGCTACCCACCAGCTGTCTTTGATTTCTGAAGCTGAATCTGTCGTTTTCTT |
| | GAACGGTGATGGTTCTATTTCTAGGGGTTCTTTCGAAGAATTGAAGAGATCTAATCC |
| | AGCCTTCAACACTTTGATGGAACACTCTAGAAAGAACGAGGATTCTGATGACGAAG |
| | AAGAAGATTTGAAGGGCGCTCCAGACGAAAAAGAATTGATTAACAGACAGTTGAC |
| | CAGACAAACCACCACTCAAATTTCTGACGATTCTAACGAATCTGGTTTGCCAGAAGG |
| | TGACGGTAAATTGATTGGAGAAGAGGAAAGATCCATTAACGCAATTGGTTGGGAT |
| | GTTTACGGTAGATATGTTTTGACCGGTGTTGATGGTTTTAAGCTGAATTGGCCAGTT |
| | TACTTGGTTTTCGGTGCTACTGTTTTTACCACGTTCTTGACTTTGTTCACGAACAACT |
| | GGTTGTCCTTTTGGATACAAATGAAGTGGGATTACTCCGACGGTTACTATATTGGTC |
| | TATACGCTATGTTTACCGCTTTGGCTGTTATGTTCATGATTACCCAATTCTGCGGTGT |
| | CATCTACATTTTGAATAGAGCCTCCAGAATCTTGAACATCAAGGCTTTGGAAAGGAT |
| | CTTGCATGTTCCAATGGCTTTTATGGATACAACTCCAATGGGTAGAGTGATCAACAG |
| | ATTCACTAAGGATACTGATACCTTGGATAACGAAATCGGTGATAGAGTTTCCATGGT |
| | CGTCTACTTTTTGTCTGATATCGTTGGCATTATCATCCTGTGCATTATCTACATGCCAT |
| | GGTTTGCTATTGCCGTTCCATTCATTATCGGTTTCTTCATTATTCTGGCTACCTTCTAT |
| | CAAGCCTCTGGTAGAGAAGTTAAGAGATTGGAAGCTATCCAAAGATCCCACGTGTA |
| | CAACAATTTCAACGAATCTTTGACTGGTATGCCAACCATTAAGGCCTTTAAGTCTATT |
| | GGTAGATTCCTGGAAAAGAACGTCAAGACGATTAACAAGATGAACGAAGCCTACTA |
| | CATTACCGTTGCTAATCAAAGATGGTTGGATGTCCATTTGTCTATGTTGGCATCATCT |
| | TTCGCTTTCTTGATTGCCATGTTGTGCGTTTTCAGAGTTTTCAATATCAACCCAGCTT |
| | CCGTTGGTTTGTTGTTGTCTTATGTCTTGCAAATCTCCTCCACCGTTTCAATGTTGGTT |
| | GTCGTTTTTACTCAAGTCGAACAGGATATGAACTCTGCCGAAAGAGTTATCGAATAC |
| | GTTTACAAGATCCCTCAAGAAAAGGCCTACGAAATCTCTGAAACAAAACCAGCTCC |
| | AGAATGGCCAGCTCATGGTGAAATCAAGTTTATTAACGTTGGTTTCGCCTACAGAG |
| | AAGGTTTGCCATTGACTTTGAAGAACTTCAACGTCGATATTAAGCCACACGAAAAG |
| | ATTGGTATTTGTGGTAGAACTGGTGCCGGTAAATCTTCTATTATGGTTGCCTTGTTC |
| | AGAATCGCTGAATTGTCTGCTGGTTCCATAGTTATTGATGGTGTTGATATCTCTACTT |
| | TGGGCTTGCATGATTTGAGATCCAGATTGTCCATTATTCCACAAGATCCAGTCTTGTT |
| | CAAGGGCACCATTAGAAAGAATTTGGATCCATTCGGTACTAAGACCGACGAAGAAT |
| | TATGGGATACTTTGAGAAGGGCCGATATCATTTCTGCTGAAACTTTGGAAGAAGTC |
| | AAGGCTCAAAAACCAGGTGATGATGATTTCAACAAGTTCCACTTGGATGGTGAAGT |
| | TGATGATGAAGGTGAAAACTTCTCATTGGGTGAAAGACAATTAGTTGCTTTTGCTA |
| | GAGCCTTGGTTAGAAACACCAAGATTTTGGTTTTAGACGAAGCCACCTCTTCAGTTG |
| | ATTATGCTACTGATTCTAAGCTGCAAAAGGCTATTGCCAGAGAATTTTCTGGTTGTA |
| | CCATTTTGTGCATTGCCCACAGATTGAAAACCATCTTGAACTACGATAGGATCATGG |
| | TTATGGACCAAGGTTCCATTTCTGAATTCGATACTCCAACGAACCTGTTCAACTCTAC |
| | ATCTTCTTTGTTCAGACAAATGTGCGACAAGTCCGGTATCTCTCAATTCGATTTTGAA |
| | GAGTAA |
| SEQ ID | MVELEKGSEPELQHRLLTPFLSKKVPPVPHDEDRPYHPKWRNPFSFLFFTWLTPVLLVG |
| NO: 960 | YKRTLLPEDMFKLHEGITAEHLAEKFQRIFDRRLAQDKQRHLKEKAKARGETLETSSVES |
| | VDDMADYELSKSLCFLTLYETFARQYSLALVFATLGMSCSTCIPLLSRKLINFVSEKAYGFN |
| | LNMGTGVGYAIGVAILIFTGDVLINQGVYLSMITGAQIRAVFTKLLLDKSFKLNAKSRKQ |
| | FPASKITAIMSTDVSRVDLGTGFSIYGFVFVFPVGISIGILVYNIKAPAMVGVGLMIAFLFV |
| | AGILGAMLFSFRKTAQKSTDARVSYMKEVLNNLKMIKFYSWEKPYFSLISKIRRREMAYL |
| | LRMEITRMIIITLASSLTLISSLASFLTLYAIASPSSRNPADIFSSVALFNMLAGQFVVLPLSI |
| | AGSTDAFLGMNRVAAVLAADEIDPKDSVRLITDDERTAMQENKLAVSVRDCDFEWEIF |
| | DLKEEKTEDQTKDNKELKKEKKEMKKKKKEEKKAQKASKSNSPSPEVDEKTGEVSSFKL |
| | NNINLDVKDGEFVVITGSIGSGKSSLLHALDGTMKKNSGKLLLNGSLLMCGVPWIQNN |
| | TLRENILFGSPYDEAWYNKVVEACSLNSDFDLLPAGDRTEIGERGITLSGGQKARVCLAR |
| | TVYEDSSIILLDDVLSAVDAKVGKHIMNECLLGLLKNKTRILATHQLSLISEAESVVFLNGD |
| | GSISRGSFEELKRSNPAFNTLMEHSRKNEDSDDEEEDLKGAPDEKELINRQLTRQTTTQI |
| | SDDSNESGLPEGDGKLIGEEERSINAIGWDVYGRYVLTGVDGFKLNWPVYLVFGATVFT |
| | TFLTLFTNNWLSFWIQMKWDYSDGYYIGLYAMFTALAVMFMITQFCGVIYILNRASRIL |
| | NIKALERILHVPMAFMDTTPMGRVINRFTKDTDTLDNEIGDRVSMVVYFLSDIVGIIILCII |
| | YMPWFAIAVPFIIGFFIILATFYQASGREVKRLEAIQRSHVYNNFNESLTGMPTIKAFKSIG |
| | RFLEKNVKTINKMNEAYYITVANQRWLDVHLSMLASSFAFLIAMLCVFRVFNINPASVG |
| | LLLSYVLQISSTVSMLVVVFTQVEQDMNSAERVIEYVYKIPQEKAYEISETKPAPEWPAH |
| | GEIKFINVGFAYREGLPLTLKNFNVDIKPHEKIGICGRTGAGKSSIMVALFRIAELSAGSIVI |
| | DGVDISTLGLHDLRSRLSIIPQDPVLFKGTIRKNLDPFGTKTDEELWDTLRRADIISAETLE |
| | EVKAQKPGDDDFNKFHLDGEVDDEGENFSLGERQLVAFARALVRNTKILVLDEATSSV |
| | DYATDSKLQKAIAREFSGCTILCIAHRLKTILNYDRIMVMDQGSISEFDTPTNLFNSTSSLF |
| | RQMCDKSGISQFDFEE* |
| SEQ ID | ATGACCGAATTGGAAAAGGCTGATCCACCAGTTTTACAAAAGAGATTATTGACGCC |
| NO: 961 | CTTCCTGTCTAAAAAGGTTCCACCTGTTCCATTGGAAGATGAACGTCCATATCATCC |
| | AAAATGGCGTAATCCATTCTCGTTCTTGTTTTTCACTTGGTTGACTCCAGTTTTGAGA |
| | AGAGGTTACAAGAGAACATTGCAGCCAGAAGATATGTTCAAGTTGCATGATCAAAT |
| | GACCGCTGAATACTTGGCTGGTAAGTTCGAAAGAATCTTCTACAGAAGGTTGGCTG |
| | CTGACAAAGAAAGACATTTGTTGCAAAAAGCCGAATCCAGAGGTGAAACTTTGGAA |
| | ACTTCTTCCGTTGATTCCGATGATGATTTCGCTGATTACCAATTGCCAAAGTCTTTGT |
| | GCTTCTTGTCCTTGTACGAAACTTTTGCTTGGCAATACTCTTTGGCTTTGTTCTTTGGT |
| | GTTTTGGGTATGTCTTGTTCTACCTGCATTCCCTTGTTGTCCAAAGAATTGATCAACT |
| | TCGTTTCCGCTAAGGCTTTTGGTATGGATGTTAATATGGGTAGAGGTGTTGGTTACG |
| | CTATTGGTGTTTCCATTTTGATCTTCACTGGTGACATCTTGATTAACCAGGGTATCTA |
| | CTTGTCTATGTTGACCGGTGCTCAAATTAGAGCTATTTTCACCAAGTTGCTGCTGGA |
| | CAAGTCTTTTAAGTTGAACACCAAGTCCAGAAAGCAATTCCCAGCTTCTAAGATTAC |
| | CTCCATTATGTCTACCGATGTTTCCAGAGTTGACTTAGGTACTGGTTTTTCTATCTAC |
| | GGCTTCATCTTCATTTTCCCAGTTGGTATTTCCATCGGTATCTTGGTTTACAATATTA |
| | GAGCACCAGCTATGGTTGGTGTCGGTTTGATGATTGCATTTTTGTTTGTTGCCGGCT |
| | TCCTGTCCTTTTTGTTGTTTTCTTTTAGACAAACGGCCCAGAAATCCACTGATGCTAG |
| | AGTTTCTTACATGAAGGAAATCCTGAACAACCTGAAGATGATCAAGTTCTACTCTTG |
| | GGAAATCCCCTACTTCAAGTTGATTTCCAAGATCAGACGTAGAGAAATGGCCTACTT |
| | GTTGAGAATGGAAATTACCCGTATGATCATTATCACCTTGGCCTCATCTTTGACCTTG |
| | ATTTCTTCATTGGCTTCGTTCCTAACCTTGTACGGTATTGCTTCTCCATCTGCTAGAA |
| | ATCCAGCAGATATTTTCTCTTCTGTTGCCCTGTTTAATATGTTGGCTGGTCAATTCGT |
| | TGTCCTGCCATTGTCTTTGGCTGGTTCTACTGATGCATTCTTGGGTATGAATAGAGTT |
| | GCTGCAGTTTTAGCTGCTGACGAAATTGATCCAAACGATTCCGTTCATATGATCACC |
| | GATTCTGAAATCACTTCCATGCAAGAAAAGAAGTTGGCCATCTCTGTTAGAGATTGT |
| | GATTTCGAATGGGAAGTTTTTAACTTCAAAGAAGAGAAGTCTGAGGATCAGACTAA |
| | GGATACTGAAGAGTTGAAGAAAGAAAAGAAAGAGCTGAAGCAGAAGAAGAAAGA |
| | AGAAAAGAAGGCCAACAAGAAGTCCAAGGGTTCTAAATCTCCAACTCCAGCTAGTG |
| | AAGAAAAAGAGGCTGAAGTTGCTTCCTTTAAGTTGCACGATATCAACTTGGATGTA |
| | CGTGATGGTGAATTCATGGTTATTACCGGTTCTATCGGTTCCGGTAAATCTTCTTTGT |
| | TGTATGCTTTGGACGGCACCATGAAGAAGAATGCTGGTAAATTGCTATTGAACGGC |
| | TCTTTGTTGATGTGTGGTGCTCCATGGATTCAAAACTCTACTTTGAGAGAAAACATC |
| | ACCTTCGGTTCTCCATATGACGAAAAATGGTACAACAAGGTTGTTAACGCTTGCTCT |
| | TTGGATTCCGATTTCGATTTGTTGCCAGCTGGTGATAGAACTGAAATTGGTGAAAG |
| | AGGTATTACCTTGTCTGGTGGTCAAAAAGCAAGAGTTTGTTTGGCTAGAACTGTTTA |
| | CGCTGACTCCTCTATTATCTTGTTGGATGATGTTTTGTCCGCTGTTGATGCTAAAGTT |
| | GGTAGGCATATTATGTCCGAATGTATCTTGGGTTTGCTGAAGGATAAGACTGTTGTT |
| | TTGGCTACCCATCAGTTGTCTTTGATTTCTGAAGCTGAATCCGTCGTTTTCTTGAATG |
| | GTGATGGTACTATTTCCAGAGGTACTTTCGATGAATTGAAGAGAACTAACTCTGCTT |
| | TCGCTACCTTGATGGAACACTCTCAAAACAACGAGGACACCGAAGAAGATTCAAAC |
| | GAACAAGGTCCAACTAACGAGAAAGAGTTGATTAACAGACAGTTGACTAGACAAA |
| | CCACCACTCAAGTTTCTGAAGAAACTGACGAAAAGAACTTCACCGAATCTGATGGT |
| | AGATTGATCATGGACGAAGAAAGATCCGTTAATGCTATTGGTTGGGATGTTTACGG |
| | CAAGTACATTTTGACTGGTGTTGAAGGTTTTAAGGCCAACTGGTTGATCTACGTTGT |
| | TTTCGCTATTACTGTCTTGACTACCTTCTTGACTTTGTTCACCAACAACTGGCTGTCTT |
| | TTTGGATCTCTATGAAGTTCGATAGATCCGACGGTTTTTACATTGGCTTGTACGCTAT |
| | GTTTACCGTTTTGGCTGTTCTGTTCATGGTTTCTCAATTCTGCGGTGTTATCTTCATCT |
| | TGAACAGAGCCTCTAGGATTTTGAACATTAAGGCCATTGAAAGGATCTTGCACGTC |
| | CCAATGTCTTTTATGGACACTACTCCAATGGGCAGAGTTATCAATAGATTCACAAAG |
| | GATACCGACACCTTGGATAATGAGATAGGTGATAGAGTCTCCATGGTCAACTACTTT |
| | TTGTCCGATTTGATCGGCATTATCATCCTGTGCATTATCTACATGCCATGGTTTGCTA |
| | TTGCCGTTCCATTCATTATTGGCCTGTTTATTATCGCTGCTACCTTCTATCAAGCTTCT |
| | GGTAGAGAAGTTAAGAGATTGGAAGCCATCCAAAGATCCCATGTTTACAACAATTT |
| | CAACGAGTCCTTATCTGGTATGCCAACCATCAAAGGTTTTGGTTCTATTGGTAGATT |
| | CCTGCAGAAGAACGTTAGCACCATTAACAAAATGTCCGAAGCCTACTTCATTACCGT |
| | TGCTAATCAAAGATGGTTGGATGTCCATTTGTCAATGTTGGCATCTTCTTTCGCTTTC |
| | TTGATCTCCATGTTGTGCGTTTTCAGAGTTTTCGATATTGGTGCTTCTTCAGTCGGTT |
| | TGTTGTTGTCCTATGTCTTGCAAATCTCCTCCATGATTTCTATGTTGGTGGTTGTTTTC |
| | ACCCAAGTCGAACAAGATATGAACTCTGCTGAAAGAGTCATCGAATACGTTTACAA |
| | GATCCCACAAGAAAATGCTTACCAGATCTCTGAAACAAAGCCTTCTCCAGAATGGCC |
| | ACAAAATGGTGAAATTAGATTCTTGAACGTTGACTTCGCTTACAGAGAAGGTTTGCC |
| | ATTGACACTGAAGAACTTTAACGCTGATATTAGGCCACACGAAAAGATTGGTATTT |
| | GTGGTAGAACTGGTGCAGGTAAGTCATCTATTATGGTTGCCTTGTTCAGAATTGCTG |
| | AATTGACTTCCGGTACTATTGAAATCGATGGTGTTGATGTTAGAACCTTGGGTTTAC |
| | ACGATTTGAGGTCCAAGTTGTCCATTATTCCACAAGATCCAGTCTTGTTCAAGGGCA |
| | CTATTAGAAAGAATTTGGACCCATTCGGTACTAAGTCTGATGATGAATTGTGGGAT |
| | ACCTTGAGAAGATCCGATATTATCTCTGCCGATAAGTTGGAAGCTGTTAAGGCTCAA |
| | AAAGTTGGTGATGATGACTACAACAAGTTCCACTTGGATTCTGAAGTTGATGACGA |
| | AGGTGAAAACTTCTCTTTGGGCGAAAAACAATTGGTTGCTTTTGCTAGAGCTTTGGT |
| | GAGAAACTCCAAGATTTTGGTTTTAGACGAAGCTACCTCCTCTGTTGATTATGCTAC |
| | TGATTCTAAGTTGCAAAAGGCTATCGCTAGAGAATTTGCTGATTGCACCATATTGTG |
| | CATTGCCCATAGATTGAAAACGATCTTGAACTACGATCGTGTTATGGTTATGGACCA |
| | GGGTGAGATCAAAGAATTCGATACTCCAAGAAACCTGTTCAACTCCAGAAACACCA |
| | TTTTCAGACAAATGTGCGATAAGTCCGGTATTTCTACTTCTGATTTCGGTGCTTGA |
| SEQ ID | MTELEKADPPVLQKRLLTPFLSKKVPPVPLEDERPYHPKWRNPFSFLFFTWLTPVLRRGY |
| NO: 962 | KRTLQPEDMFKLHDQMTAEYLAGKFERIFYRRLAADKERHLLQKAESRGETLETSSVDS |
| | DDDFADYQLPKSLCFLSLYETFAWQYSLALFFGVLGMSCSTCIPLLSKELINFVSAKAFGM |
| | DVNMGRGVGYAIGVSILIFTGDILINQGIYLSMLTGAQIRAIFTKLLLDKSFKLNTKSRKQF |
| | PASKITSIMSTDVSRVDLGTGFSIYGFIFIFPVGISIGILVYNIRAPAMVGVGLMIAFLFVAG |
| | FLSFLLFSFRQTAQKSTDARVSYMKEILNNLKMIKFYSWEIPYFKLISKIRRREMAYLLRM |
| | EITRMIIITLASSLTLISSLASFLTLYGIASPSARNPADIFSSVALFNMLAGQFVVLPLSLAGS |
| | TDAFLGMNRVAAVLAADEIDPNDSVHMITDSEITSMQEKKLAISVRDCDFEWEVENFK |
| | EEKSEDQTKDTEELKKEKKELKQKKKEEKKANKKSKGSKSPTPASEEKEAEVASFKLHDIN |
| | LDVRDGEFMVITGSIGSGKSSLLYALDGTMKKNAGKLLLNGSLLMCGAPWIQNSTLRE |
| | NITFGSPYDEKWYNKVVNACSLDSDFDLLPAGDRTEIGERGITLSGGQKARVCLARTVY |
| | ADSSIILLDDVLSAVDAKVGRHIMSECILGLLKDKTVVLATHQLSLISEAESVVFLNGDGTI |
| | SRGTFDELKRTNSAFATLMEHSQNNEDTEEDSNEQGPTNEKELINRQLTRQTTTQVSEE |
| | TDEKNFTESDGRLIMDEERSVNAIGWDVYGKYILTGVEGFKANWLIYVVFAITVLTTFLT |
| | LFTNNWLSFWISMKFDRSDGFYIGLYAMFTVLAVLFMVSQFCGVIFILNRASRILNIKAIE |
| | RILHVPMSFMDTTPMGRVINRFTKDTDTLDNEIGDRVSMVNYFLSDLIGIIILCIIYMPW |
| | FAIAVPFIIGLFIIAATFYQASGREVKRLEAIQRSHVYNNFNESLSGMPTIKGFGSIGRFLQ |
| | KNVSTINKMSEAYFITVANQRWLDVHLSMLASSFAFLISMLCVFRVFDIGASSVGLLLSY |
| | VLQISSMISMLVVVFTQVEQDMNSAERVIEYVYKIPQENAYQISETKPSPEWPQNGEIR |
| | FLNVDFAYREGLPLTLKNFNADIRPHEKIGICGRTGAGKSSIMVALFRIAELTSGTIEIDGV |
| | DVRTLGLHDLRSKLSIIPQDPVLFKGTIRKNLDPFGTKSDDELWDTLRRSDIISADKLEAVK |
| | AQKVGDDDYNKFHLDSEVDDEGENFSLGEKQLVAFARALVRNSKILVLDEATSSVDYAT |
| | DSKLQKAIAREFADCTILCIAHRLKTILNYDRVMVMDQGEIKEFDTPRNLFNSRNTIFRQ |
| | MCDKSGISTSDFGA* |
| SEQ ID | ATGGATCATGAATCCGCTGCTTTTTCATCTAGAGCACCACCATTGAGACAAAACAGA |
| NO: 963 | TTATTGTCTCCTCTGTTCACCAAAAAGGTTCCACCAGTTCCACAAGATCACGAAAGA |
| | CATACTTATCCCTTGTACGGTAATCCAATCTCCTGGTTTTTCTTTACTTGGTTGTGGCC |
| | AGTTATGATCACTGGTTACAAAAGAACTTTGGAACCAGACGACCTGTACAAGTTGA |
| | ATGATAAGTTGAAAGCTGATGCTTTGGCTGCTAGATTCGAAGCTATTTTTGCTAGAA |
| | GATTGGCCGAAGATAAGAGAAGGCATTTGGATCAAACTCTGGACTCCTCTAAGATC |
| | TCTAACTCTTCTAAGAACTCCTCCAACTCTCCAGATTTGGATGATTTGGCCGATTTGG |
| | CTGACTATGTTCCATCTGATACTTTGTGTTTGTGGTCCTTGTTCGAAACTTTCAAGTG |
| | GCAATATTTGACCGCTTGTTTCTTGTGTGCTTTAGCTCAAGTTGGTTGGACTTGTAAT |
| | CCTCTGTTGTCCAAGAAATTGATCGCCTACGTTCAAAGAAAGGCCTTGGGTATTGAA |
| | TCTGATACAGGTAAAGGTGTTGGTTACGCTTTGGGTGTTTCTTTGGTTGTTTTCTGCT |
| | CCGACATCTTGTTTAACCAGATGTACTACTTGTCCTCTTTGACTGGTGCTGAATCTAA |
| | GGCTATTTTCACCAAAGTTATGCTGGACAAGTCCTTCAGATTGAATGCTAGATCAAG |
| | AAGAGTTTACCCCGTTTCCAAGATTACCTCTATTATGTCTACCGATGTCTCCAGAATC |
| | GATTTGGGTTTAGCTACTGCTCCAATGATTATAGTTGCTCCAGTTCCATTGGCCATTT |
| | CCATTGGTATTTTGATCCATAACTTGAAGGCTCCAGCCTTGTTAGGTATTGGTATCAT |
| | GATTTTGTTCTTGGGTTTCGCTGGTTTCTTGGGTAGTTTGTTGTTTAAGTACAGAAA |
| | GTTGGCTACTACCCAAACCGATGCTAGAGTTTCTTATATGAAGGAAGTCCTGAACAA |
| | CCTGAAGATGATCAAGTTTTACTCTTGGGAAAAGCCCTACATGGCTATGATTAAGGC |
| | TGTCAGAGAAAAAGAGATGACCTTCTTGTTGAAGATGCAAGTCACCAGATCCATCA |
| | TTATTTCCGTTGCTGTTTCCTTGTCTTTGGTAGCTTCTTTTGCTAGCTTCATGTTGTTG |
| | TATGGTACTGCCTCTGTTTCTAAGAGAAATCCAGCTTCTATCTTCTCTTCTGTTGCCCT |
| | GTTCAATATTTTGGCCTCCGTTTTTATCAACCTGCCATTGGCTATTGCTGGTGCTACT |
| | GATGCTTATATTGGTATGAGAAGAGTCGGTCAATACTTGGCTTCTGATGAACACGTT |
| | GAGGATAAGAAGAGAGTTACTTCTGAAACCGACAGACAATTGATGGAAGAAAAGA |
| | ATTTGGCCATCACCGTTTCTAACGCTAATTTCGAATGGGAAATCTTCGATATCCCAG |
| | ACGAGGAAAAGATCAAAGAAGAAAAGAAGAAACAAAAGGACAAAGAGAAGAACG |
| | ACAAAAAGAACAAGAAAAAGAAGTTGTCCTCCGACGAATCTTCTCATGAAGCTGTT |
| | ACACAATCTGAAAAGCCAACTTCTGCTGCTACCTTTAAGTTGAGAAACATCGATTTG |
| | ACCATCATGAAGGGTGAATTCGTTGTTGTTACTGGTTCTATCGGTTCCGGTAAATCA |
| | TCTTTGTTGTTGGCTTTGGAAGGTTCCATGAAGAGAAATTCTGGTCAAGTTAAGACC |
| | AACGGCTCTTTGTTGATGTGTGGTGCTCCATGGATTCAATCCTCTACTATTAGAGAA |
| | AACGTCATTTTCAACAACCCCTACAACAAGTCTTGGTACGAACAAGTTATTGATGTT |
| | TGCTGCATGGACTCCGATTTGGAAATTTTGCCAGCTGGTGATCAAACCGAAATAGG |
| | TGAAAGAGGTATTACTTTGTCTGGTGGTCAAAAGGCTAGATTGTCTTTAGCTAGAG |
| | CTGTTTACGCTAGATCCGATATCATTTTGTTGGATGATGTCTTGTCTGCTGTTGATGC |
| | TAAAGTTGGTAAGAGAATCGTTGACGAATGTATCTTAGGTGTCTTGAGAAAGAAAA |
| | CCGTTGTTTTGGCTACTCACCAGTTGTCCTTGATTGAATCAGCAGATAAGATCGTGT |
| | TCTTGAATGGTGATGGTACAGTTGATGTTGGTACTTCCGAATCTTTGAGAAGATCTA |
| | ACGAAGCCTTCCAGAAGTTGTTGTCTCATTCTACTACTGAAAAGTACGCCGAAGAG |
| | GAATCCTCTATTTCTTCACAAACTGACGAGTCCATCAAGAAGGTTGTTGTTGAAGCT |
| | CAGATTTCCAGATTGACTTCCGTTTCTTCAACTAACGAAAAGACCGACTTGCAAAAG |
| | CAGAACGAAGGTAAATTGATCATGGAAGAGGAAAAGTCCGTTAACGCTATTAACGC |
| | TGATGTTTACGTTAGGTACATTTTCGCAGGTATTCCAGGTGTTAAGGGTGCTATGAT |
| | TTTTGCTGCTGTTATCATCTTCTCCATCCTGTCCGTTTTCTTTAACTTATTCACTTCCAC |
| | CTGGTTGTCTTTTTGGGTTGAGTACAAATGGCGTAATAGATCTGATGGTTTCTACAT |
| | TGGTTTTTACGCTGCTTTCACAGTTTTGGCCTTGGTTACTTTGACTTTTGGTTTCTCTG |
| | GTGTCATCTACGTCATGAACTTATCTTCTAGAACCTTGAACATTAGAGCCGCCGAAA |
| | GAATCTTGTATGTTCCAATGTCTTACATGAACGTTACCCCAATGGGTAGAATCATTA |
| | ACAGATTCACTAAGGATACCGACGTGTTGGATAACGAAATGGGTGATAGAATGGG |
| | TATGATTATCTACTTCGCCTCCATTATTGGTGGCGTTTTGATTTTGTGTATCATCTACT |
| | TGCCATGGTTCGCTATAGCTGTTCCATTTTTGATTGTCGTTTTCTTCGGTTTTGCTAAC |
| | TTCTACCAAGCTTCTGGTAGAGAAATCAAGAGATTGGAAGCTGTTCAAAGGTCCTT |
| | GGTCTACAACAATTTCAACGAAACTTTGACCGGTTTGGATACCATTAGAGGTTACGA |
| | TAAGACCGATGTGTTCCTGTCTAAGAACATCAGATTGATCGACAAGATGAACGAGG |
| | CTTACTTCATTACCGTTGCTAATCAAAGATGGTTGGATGTTGCAGTTTCTTTCTTGGC |
| | TACAATTTTCGCCATCATCATCTCATTCTTGTGCGTGTTTAGAGTGTTCAAGATTAAC |
| | GCTTCTTCCGTTGGTTTGCTGTTGTCTAATACCTTGCAAATCTCCGGTATTATCACCA |
| | CATTGGTTGTCGTTTACACCAGAGTTGAACAAGATATGAACTCCGCTGAAAGGATC |
| | ATCGAATACGTTGATGATTTGCCACAAGAAGCTCCATACACCATTTCTGAAACTACT |
| | CCAAATCCATCTTGGCCTCAACAAGGTCAAATTGACTTTAACCATGTTAACTTGGCTT |
| | ACAGACCAGGTTTGCCAATGGTTTTGAAGGATTTCACCGTTCATATTGACCCAAACG |
| | AGAAGATCGGTATTTGTGGTAGAACAGGTGCTGGTAAATCTTCTATTATGGTTGCCT |
| | TGTACAGAATGGTCGAATTGACCTCTGGTAACATTACCATTGATGGTATCGATATCA |
| | GAACCTTGGGCTTGAACAATTTGAGGTCCAAGTTGTCCATTATTCCACAGGATCCAG |
| | TTTTGTTCCAAGGCACTATTAGAAAGAACTTGGATCCATTTGGTTCCGCTACAGATG |
| | AACAATTGTGGGAAACTTTAAGAAGGGCCAGAATCATCAAGTCCGAAGATTTGAAT |
| | GAAGTCAAGTCTCAAACCGATCCTAACAAGATGCATAAGTTCCACTTGGATAGAGA |
| | TGTTGATGTCGATGGTGAAAACTTCTCCTTGGGTGAAAAACAATTGATTGCTTTTGC |
| | CAGAGCTTTGGTCAGAGGTTCTAAAATCTTGATTTTGGATGAAGCCACCTCCTCAGT |
| | TGATTATGCAACCGATAAGATATTGCAAGAAGCCATCGTTGAAGAATTCTCCGATTG |
| | CACTATTTTGTGCATTGCCCATAGACTGAAAACCATCTTGAACTACGATAGAGTTAT |
| | GGTCATGGATCAAGGTCAAGTTGTTGAATTCGATAAGCCCATCAACCTGTTTAAGA |
| | AGCAAGGTACTTTCTTCCAGATGTGTGAAAAGGCTGGTATCAACGAAAAAGAATTC |
| | GGTCACTGA |
| SEQ ID | MDHESAAFSSRAPPLRQNRLLSPLFTKKVPPVPQDHERHTYPLYGNPISWFFFTWLWP |
| NO: 964 | VMITGYKRTLEPDDLYKLNDKLKADALAARFEAIFARRLAEDKRRHLDQTLDSSKISNSSK |
| | NSSNSPDLDDLADLADYVPSDTLCLWSLFETFKWQYLTACFLCALAQVGWTCNPLLSKK |
| | LIAYVQRKALGIESDTGKGVGYALGVSLVVFCSDILFNQMYYLSSLTGAESKAIFTKVMLD |
| | KSFRLNARSRRVYPVSKITSIMSTDVSRIDLGLATAPMIIVAPVPLAISIGILIHNLKAPALL |
| | GIGIMILFLGFAGFLGSLLFKYRKLATTQTDARVSYMKEVLNNLKMIKFYSWEKPYMAM |
| | IKAVREKEMTFLLKMQVTRSIIISVAVSLSLVASFASFMLLYGTASVSKRNPASIFSSVALF |
| | NILASVFINLPLAIAGATDAYIGMRRVGQYLASDEHVEDKKRVTSETDRQLMEEKNLAIT |
| | VSNANFEWEIFDIPDEEKIKEEKKKQKDKEKNDKKNKKKKLSSDESSHEAVTQSEKPTSA |
| | ATFKLRNIDLTIMKGEFVVVTGSIGSGKSSLLLALEGSMKRNSGQVKTNGSLLMCGAPW |
| | IQSSTIRENVIFNNPYNKSWYEQVIDVCCMDSDLEILPAGDQTEIGERGITLSGGQKARL |
| | SLARAVYARSDIILLDDVLSAVDAKVGKRIVDECILGVLRKKTVVLATHQLSLIESADKIVFL |
| | NGDGTVDVGTSESLRRSNEAFQKLLSHSTTEKYAEEESSISSQTDESIKKVVVEAQISRLTS |
| | VSSTNEKTDLQKQNEGKLIMEEEKSVNAINADVYVRYIFAGIPGVKGAMIFAAVIIFSILS |
| | VFFNLFTSTWLSFWVEYKWRNRSDGFYIGFYAAFTVLALVTLTFGFSGVIYVMNLSSRTL |
| | NIRAAERILYVPMSYMNVTPMGRIINRFTKDTDVLDNEMGDRMGMIIYFASIIGGVLIL |
| | CIIYLPWFAIAVPFLIVVFFGFANFYQASGREIKRLEAVQRSLVYNNFNETLTGLDTIRGYD |
| | KTDVFLSKNIRLIDKMNEAYFITVANQRWLDVAVSFLATIFAIIISFLCVFRVFKINASSVGL |
| | LLSNTLQISGIITTLVVVYTRVEQDMNSAERIIEYVDDLPQEAPYTISETTPNPSWPQQGQ |
| | IDFNHVNLAYRPGLPMVLKDFTVHIDPNEKIGICGRTGAGKSSIMVALYRMVELTSGNIT |
| | IDGIDIRTLGLNNLRSKLSIIPQDPVLFQGTIRKNLDPFGSATDEQLWETLRRARIIKSEDL |
| | NEVKSQTDPNKMHKFHLDRDVDVDGENFSLGEKQLIAFARALVRGSKILILDEATSSVD |
| | YATDKILQEAIVEEFSDCTILCIAHRLKTILNYDRVMVMDQGQVVEFDKPINLFKKQGTF |
| | FQMCEKAGINEKEFGH* |
| SEQ ID | ATGGCCGAATTGGAAAAGGGTGATGATGCTCAACCAGCTTTACAACATAGATTGTG |
| NO: 965 | TACACCCTTGTTGTCCAAAAAGGTTCCACCAGTTCCAAGAGATGAAGATAGACCAG |
| | TTCATCCAAAAGCTACCAATCCATTCTCTTGGTTTTTCTTCACTTGGTTGACTCCAGTT |
| | TTGTTGAGAGGTTACAAGAGAACTTTGTTGCCAGAGGATATGTTCAAGTTGCATGA |
| | CGAAATGACCGTTGAACATTTGGCTGGTAAGTTCCAAGCTATCTTCGATAGAAGATT |
| | GGCTGCTGATAAGAGGAAGTACTTGAAAAAGAGAAGGCAATTGGCCTTGAAGAAA |
| | GGTGATTCTGATGTTTCTGCTAGAACCGACGAAGATTTGATGTTGGAATACGAACC |
| | ATCTAAGTCCCTGTGTTTCTTGTCCTTGTACGAAACATTTTTGTGGCAATACTCCATG |
| | GCTTTGTTGTTCGGTATGTTGGGTTTAGTTGGTCAAGCTTGTAATCCTTTGTTGTCCC |
| | GTAAGTTGATTAACTTCGTTGAATTGGAAGCCTTGGGTATCCCAACAAAAATTGGTA |
| | CTGGTATTGGTTACGCTTTCGGTGTTGCTATTTTGATGTTTGTTTCCGACGTCTTGCA |
| | CAATCAAGGTGTTTATTTTGCTATGTTGACCGGTGCTCAAATTAGAGCTATTTTCACA |
| | AAAGCCCTGCTGGACAAGTCTTTCAAATTGAATACCAGATCGAGGAAGAAGTTCCC |
| | ACCATCTAAAATTACCTCCATCATGTCTACCGATGTTTCCAGAGTTGACTTAGGTACA |
| | GGTTTTTCTATCTACGGTTTCGTTTTGATCGTTCCAGTTGGTGTTTCCATTGGTATCTT |
| | GATCTACAACATTAAGGCTCCAGCTATGGTTGGTGTTGGTTTGATGTTAGCTTTCTT |
| | GTTCGTTTCTGGTGGTTTGTCTACCTTGTTGTTCTCTTTCAGAAAAACCGCTCAAAAG |
| | GCTACCGATTCTAGAGTTGGTTATATGAAGGAAGTGCTGAACAACCTGAAGATGAT |
| | CAAGTTTTACTCTTGGGAAAAGCCATACCATGCCTTGATTACTAAGATCAGACGTAG |
| | AGAAATGGCCTACTTGTTGAGAATGGAAATTACCCGTATGATCATTATTACCTTGGC |
| | TGCTTCTTTGGCCTTGGTTTCATCTTTGGTTTCTTTCTTGACGTTGTACGCTATTGCTT |
| | CTCCATCTTCTAGAAATCCAGCCGAAATTTTCTCCTCCGTTTCTTTGTTTAACTTGTTG |
| | GCCTCTCAATTCTTGGTCTTGCCATTGTCTATTGCTGGTTCTACTGATGCATTTTTGG |
| | GTATGAATAGAGTTGCTGCTGTTTTAGCTGCTGACGAAATTGATCCAGAAGATGCT |
| | GATACCATTTTGTCAGAAAGAGCACAAGCTTTGTTGGAAGAAAAGAAGTTGGCTAT |
| | CACTGTTCAAGACGGTGAATTCGAATGGGAGTTGTTTGATTTTGACGACGAAAAGT |
| | CTGAAGAAACCGAAGAACATAAGGACGAGTCCAAGAAAAAGAAGATGAAGAAAG |
| | AGTCGAAGAAGAAGGTCAAGAAGTCCACCAAAGATATCTCTGATACTTCTTCCGCTT |
| | CCTCCAATGAGAAAGAGAGAAAGTCTTTTAAGCTGCACAACGTGAACTTGGATATT |
| | AGACAAGGTGCCTTCGTTGTTATTACCGGTTCTATTGGTTCTGGCAAGTCATCTTTGT |
| | TGCATGCTTTGGATGGTGCCATGAAGAAATTGTCTGGTGATGTTTACGTTAACGGCT |
| | CTTTGTTGATGTGTGGTACTCCTTGGATTCAATCTGCTTCATTGAGAGAGAACATCTT |
| | GTTTGGTTCTACCTATGACGAAACCTGGTACAAAGAAGTTATTAGAGCTTGCTTCTT |
| | GGAGTCCGATTTCGATATTTTGCCAGCTGGTGATTTGACCGAAATTGGTGAAAGAG |
| | GTATTACTTTGTCCGGTGGTCAAAAAGCTAGAGTTTGTTTGGCTAGAACTGTTTACG |
| | CTAACTCCTCCATTATCTTGTTGGATGATGTTTTGTCTGCTGTTGATGCCAAGGTTGG |
| | TAAACATATTATGTCCGAATGCATCATGGGTATCTTAAAGGGTAAGACTAGAGTTTT |
| | GGCTACCCACCAATTGTCCTTGATTTCTGAAGCTGAACACGTCATTTTCTTGAATGGT |
| | GATGGTACTATCTCCAGGGGTACTTTTGAAGAATTGAAGTCTACCAACTCTGCCTTC |
| | AAGGCTTTGATGGAACATAACCGTAAATCCGAAGAAAACGATGAGGATGAATCTG |
| | AACCAGCATCCGAATTAGAAGTCTCCGAAAAAGAATTGATCAAGAGGCAATTGACT |
| | AAGCAGACTACTACCCAAGTTTCCTCTGATTCTGTTGAAAAGGTAGAATTGGATGGC |
| | AAGTTGTACGATGAAGAAGAAAAATCCGTTAACGCCATTGGTTGGGATGTTTATGG |
| | TCGTTACATTTTGACTGGTGTCCAAGGTTTTAAGTTCAACTGGTTGTTGTTTGTCATC |
| | CTGTGCTTGTGTATTTTGGGCACTTTTATGTCTCTGTTCACCAACAATTGGCTGTCTT |
| | TTTGGATCTCCAGAAAGTTTGATCAATCCGCCGGTTTTTACATTGGTTTCTACGCTAC |
| | TTTTACCGGTTTGGCTGTTATCTTGATGGTGTTCCAATTCTGCTCCATCATCTTCGTTA |
| | TGAACAGAGCCTCTAGGATCTTGAATATTAAGGCTTTGGGTAAGATCTTGCACGTCC |
| | CAATGTCTTTCATGGATACAACTCCAATGGGTAGAATCCTGAACAGATTCACTAAGG |
| | ATACTGACACCTTGGATAACGAAATCGGTGATAGAGTCGGTATGGTTGTTAACTTC |
| | ACTTTCGAAATTTGGGGCGTTATCATCATGTGCATTATCTACATGCCATGGTTTGCTA |
| | TTGCTGTTCCTTTTATCGTTGCCGTGTTCATTATTATGGCTAACTTCTATCAAGCCTCC |
| | GGTAGAGAAGTTAAGAGATTGGAAGCTGTTCAAAGATCCCACGTTTACAACAACTT |
| | CAACGAATCTTTAACTGGTATGCCAACCATTAAGGCCTTCAAGTCCATTCAGAGATT |
| | CTTGAACAAGAACATTGCCACGATCAACAAGATGGATGAGGCTTACTTTGTTACCGT |
| | TGCTAATCAAAGATGGTTGGACACTTACTTGTCTTTGTTGGCTACTTTGTTCGCCTTG |
| | TTGATTGCTTTGTTATGTGCTTGCAGAGTGTTCGATATTGGTGCTTCTGCAGTTGGTT |
| | TGTTAGTTTCCTACGTTCTGCAAATCTCCGGTTTGATTTCTATGTTGGTTGTCGTTTTC |
| | ACCCAAGTCGAACAAGATATGAATTCTGCTGAAAGAGTCCTGGATTACGTCTACAA |
| | AATTCCACAAGAAAGGCCATACGAAATCTCCGAAACTAGACCACCACCTGAATGGC |
| | CTCAAAATGGTGAAATTCAATTCATCAACGTCGACTTCGCTTACAGAGAAGGTTTGC |
| | CATTGACTTTGAGAAACATTAACGCCGATATTAAGCCACACGAAAAGATTGGTATTT |
| | GCGGTAGAACTGGTGCTGGTAAATCTTCAATTATGGTCGCTTTGTTCAGAATCGCCG |
| | AATTGACTTCTGGTTCCATTATGATTGATGGCATCGATGTTTCTACTTTGGGCTTGCA |
| | TGAATTGAGGTCCAACTTGTCTATTATTCCACAGGATCCAGTCTTGTTTAAGGGCAC |
| | TATTAGGTCTAATTTGGACCCATTTGAAACTAAGACCGATGACGAATTGTGGGATAC |
| | TTTAAGAAGGGCTGATATTATTGATGCCACCTCTTTGGAACATGTTAAGACTCAACG |
| | TGTTGGTGATGACGATTTCCATAAGTTCCACTTGGATAATGAGGTTGATGACGAAG |
| | GTGAAAACTTCTCTCTAGGTGAAAAACAATTGGTTGCTTTCGCTAGAGCTTTGGTTA |
| | GAGATACCAAGATTATCGTTTTGGACGAAGCTACCTCTTCTGTTGATTATGCTACTG |
| | ACTCTAAGTTGCAAAAGGCCATCGTTAGAGAATTTTCCGATAGAACCATATTGTGCA |
| | TTGCCCACAGATTGAAAACCATCTTGCATTACGATAGAGTCATCGTCATGGAACAG |
| | GGTGAAATCAAAGAATTCGATACTCCATCTCACCTGTACAACTCTACTGGTACTATTT |
| | TCAGACAAATGTGCGACAAGTCCGGCATCTCTAAAGAAGATTTTTACGAGTGGTAA |
| SEQ ID | MAELEKGDDAQPALQHRLCTPLLSKKVPPVPRDEDRPVHPKATNPFSWFFFTWLTPVL |
| NO: 966 | LRGYKRTLLPEDMFKLHDEMTVEHLAGKFQAIFDRRLAADKRKYLKKRRQLALKKGDSD |
| | VSARTDEDLMLEYEPSKSLCFLSLYETFLWQYSMALLFGMLGLVGQACNPLLSRKLINFV |
| | ELEALGIPTKIGTGIGYAFGVAILMFVSDVLHNQGVYFAMLTGAQIRAIFTKALLDKSFKL |
| | NTRSRKKFPPSKITSIMSTDVSRVDLGTGFSIYGFVLIVPVGVSIGILIYNIKAPAMVGVGL |
| | MLAFLFVSGGLSTLLFSFRKTAQKATDSRVGYMKEVLNNLKMIKFYSWEKPYHALITKIR |
| | RREMAYLLRMEITRMIIITLAASLALVSSLVSFLTLYAIASPSSRNPAEIFSSVSLFNLLASQF |
| | LVLPLSIAGSTDAFLGMNRVAAVLAADEIDPEDADTILSERAQALLEEKKLAITVQDGEFE |
| | WELFDFDDEKSEETEEHKDESKKKKMKKESKKKVKKSTKDISDTSSASSNEKERKSFKLH |
| | NVNLDIRQGAFVVITGSIGSGKSSLLHALDGAMKKLSGDVYVNGSLLMCGTPWIQSASL |
| | RENILFGSTYDETWYKEVIRACFLESDFDILPAGDLTEIGERGITLSGGQKARVCLARTVY |
| | ANSSIILLDDVLSAVDAKVGKHIMSECIMGILKGKTRVLATHQLSLISEAEHVIFLNGDGTI |
| | SRGTFEELKSTNSAFKALMEHNRKSEENDEDESEPASELEVSEKELIKRQLTKQTTTQVSS |
| | DSVEKVELDGKLYDEEEKSVNAIGWDVYGRYILTGVQGFKFNWLLFVILCLCILGTFMSL |
| | FTNNWLSFWISRKFDQSAGFYIGFYATFTGLAVILMVFQFCSIIFVMNRASRILNIKALGK |
| | ILHVPMSFMDTTPMGRILNRFTKDTDTLDNEIGDRVGMVVNFTFEIWGVIIMCIIYMP |
| | WFAIAVPFIVAVFIIMANFYQASGREVKRLEAVQRSHVYNNFNESLTGMPTIKAFKSIQR |
| | FLNKNIATINKMDEAYFVTVANQRWLDTYLSLLATLFALLIALLCACRVFDIGASAVGLLV |
| | SYVLQISGLISMLVVVFTQVEQDMNSAERVLDYVYKIPQERPYEISETRPPPEWPQNGEI |
| | QFINVDFAYREGLPLTLRNINADIKPHEKIGICGRTGAGKSSIMVALFRIAELTSGSIMIDG |
| | IDVSTLGLHELRSNLSIIPQDPVLFKGTIRSNLDPFETKTDDELWDTLRRADIIDATSLEHV |
| | KTQRVGDDDFHKFHLDNEVDDEGENFSLGEKQLVAFARALVRDTKIIVLDEATSSVDYA |
| | TDSKLQKAIVREFSDRTILCIAHRLKTILHYDRVIVMEQGEIKEFDTPSHLYNSTGTIFRQM |
| | CDKSGISKEDFYEW* |
| SEQ ID | ATGGCTTCCTCTTCTGCCTTGGAATCCAATCAATTGTCTTTGGAAAGACAGAAGAGG |
| NO: 967 | CTGTTGTCTTTCTTGATGTCTAAAAACGTTCCACCAGTGCCAACTCAAGAAGAAAGA |
| | AAACCATATCCAGGTGCTACTGCCAACGTTATTAAGCAAATTTTCTTTTGGTGGTTG |
| | GCCCCAGTTATGAATACTGGTTATCAAAGAACATTGCAGCCAGACGATATGTTCTAC |
| | TTGACCGATGATATCAAGGTTCAAAAGATGGCCGATGATTTCTACAGGTACATGTCC |
| | AACGATATCGACAGATCCAAGCAAAAACATATTGCTGCCAAGTGCAAAGAAAGAG |
| | GTGAAACTGTTGAAACCACTTCCGTTGATCCTGAAGAGGATTTGAAAGACTACGAG |
| | TTGTCTAAGTTCTTGACCGTTTGGGCTTTAGCTAAGACTTTTAAATGGCAGTATACCT |
| | GGGCTTGTGTTTGTTTGGCTTTGTCTAATGCTGGTCAAACTACTATGCCTCTGCTGTC |
| | TAAGAAGTTGATCAAGTACGTTGAATTGAAGGCTTATGGACAAGAACCAGGTATTG |
| | GTAAAGGTTTGGGTTACTCTTTTGGTACTACCGCCATGGTTTTTATCGTTGGTGTTTT |
| | GATCAACCACTTCTTCTACAGATCTATGATTACTGGTGCTCAAGCTAAAGCTGTTTTG |
| | ACTAAGGCTTTGTTGGACAAGTCCTTTAAGTTGTCTGCTGAAGCTAAACACAAGTAC |
| | TCCGTTGGTAAGATTACTTCCATGTTGGGTACTGACTTGTCTAGAATTGATTTCGCTT |
| | TGGGTTTCCAGCCATTCATTATCGTTTTCCCAATTCCAATTGCTATCGCCATTGCCATT |
| | TTGATCGTTAATATTGGTGTTGCCTCTTTGGTTGGTGTTGGTATTTTGTTGGTTTTCA |
| | TGGTTGGTATTGCTTTCTCTACCGGTAAGTTGATTGCTTACAGAAAGAAGGCTAACC |
| | ACTACACCGATTCTAGAGTCAACTACATCAAAGAAGCCCTGAACAACTTGAAGGTC |
| | ATCAAGTTTTATTCTTGGGAACCACCATACCACGAAAACATTTCTGACCTGCGTAAG |
| | AAAGAGATGAAGATCATCTACAGAATGCAGGTCTTGAGAAACGTTGTTACTTCTTTC |
| | GCTATGTCCTTGACTTTGTTCGCTTCTATGACTGCATTTTTGGTCTTGTACGCTATCTC |
| | CAATGGTAAAAGAGATCCAGCCTCTATCTTCTCCTCTTTGTCCTTGTATAACACTTTG |
| | ACCCAACAGGTTTTCTTGTTGCCAATGGCTTTGGCTACAGGTGCTGATGCTTTTATG |
| | GGTATTTCTAGAGTTGGTGACTTCATGTCCCAATCTGAAATCAATCCAGAAGAAAAC |
| | GCTATTGAAGCTCCACCAGATGTTCAAGAATGGATGGACAAAGAATCCTTGTCTATC |
| | GATGTTGAAGATGCCTCTTTCGAATGGGAAATTTTCGAAGATGATGAAGAGAAGGA |
| | CGGCAAGAATTCTAAGAAGAACGGTTCCAAAAAGAGGAAGTCCGAAGAAAACATC |
| | ATCTTGGATGTGCAAGACTCCGAATCTTCTTTGACAAAAGGTTTAGACGGCTCCTCT |
| | AGTTCTGAAGTTTCATCAGCTGAAGATGCTCACTTTGAAGGTTTGAACAACATCGAC |
| | TTCAAGATCAAGAAGGGTGAATTCGTTGTTATCACCGGTTTGATTGGTTCCGGTAAA |
| | TCATCTTTGTTGTCAGCTATGTCCGGTTTTATGAAGAGGACTTCTGGTTCCGTTAATG |
| | TCAATGGTAGTTTGTTGTTGTGTGGTTACCCTTGGGTTCAAAACTCTACTGTTAAGG |
| | ACAACATTATCTTCGGTTCCGAATTCGACGAAGAGAAGTACAAAAAGGTTATCTAC |
| | GCTTGCTCTTTGGAAGCTGACTTGGAATTATTGCCAGCTGGTGATAGAACTGAAATT |
| | GGTGAAAGAGGTATTACCTTGTCTGGTGGTCAAAAGGCTAGAATCAATTTGGCTAG |
| | AGCTGTTTACGCTAACAAGGACATCATTCTGTTGGATGATGTCTTGTCAGCTGTTGA |
| | TGCAAGAGTTGGTAAACACATTATGAACAACTGCCTGCTGGATCTGTTGAAAGAAA |
| | AGACAAGAGTTTTGGCTACCCACCAGTTGTCTTTAATTGGTTCTGCTGATAGAGTCA |
| | TCTTCTTGAATGGTGATGGTTCCATTGATGTCGGTACTTTCGAAGAATTGAAAGAGA |
| | GAAATCCAGGCTTCGATAAGTTGATGGCATACAACTCTGAATCCCATGAGGAAGAT |
| | GAAGAAGAAGAAGAGAACTTGGAAGAAATCGCTGAAGAGGACCTATTTGAAGATG |
| | ACAAGAGAGTTGTTGAAAGGCAGTTGACTAGAAGATCTACTAGAACTGTTACATCC |
| | GTTGGTGCTTTTCCCGAAGAAGAATACGACGAAGCAGATGAAGAGGCTAGACATC |
| | ATGAGTACAATTTGGATGAACAAGCCGACGGTAAATTGATCGGTGATGAAGAAAG |
| | AGCAGTTAACGCCATCTCTAAAGAAATCTACGCTAGGTACTTCGAACTAGGTTCTGG |
| | TAGATTGACTCCATGGGTTATGTTGCCTTTGCTGTTGTTTTTCATTGTCGTTGCTACC |
| | TTCTCTCAGATTTTCACTAATACGTGGTTGTCCTTTTGGACCGAGTACAAATTTGATA |
| | AGCCAGACAAGTTCTACATCGGCATCTATATTATGTTCGCCTTCCTGTCCTTCATCTT |
| | GTTGACCATTGAATTCATCATCCTGGTCAAGATTACCAACACTGCTTCTGTTATGATG |
| | AACGTTTTGGCCGTTAAGAAAGTTTTACATGCCCCAATGTCTTTCATGGATACCACTC |
| | CAATGGGTAGAATCTTGAACAGATTCACTAAGGATACCGATGTCTTGGATAACGAA |
| | ATCGGTGATCAATTGAGGTTCTTCTTGTACGTTTTCGCCAACATTATTGGTGTCGTTA |
| | TCTTGTGCATTATCTACTTGCCTTGGTTTGCTATTGCTGTCCCATTTTTGGGTATGCTG |
| | TTCGTTTCTATCTCTAACTACTATCAAGCTTCCGCCAGAGAAATCAAAAGATTGGAA |
| | GCAGTTCAAAGGTCCTTGGTCTACAACAATTTCAACGAAACTTTGTCTGGTATGGCT |
| | ACCATTAAGGCTTACAAAGCTACCGAAAGATTCATCGGTAAGAACAACTACTTGATT |
| | GACAGAATGAACGAGGCCTACTACATTACCATTGCTAATCAAAGATGGTTGGCCAT |
| | CCACATGGATTTTGTTGCTACTTTATTCGCCTTGCTGATTGCTTTGTTGTGCGTTAAT |
| | AGAGTGTTTAACGTTTCCGCTTCTGCTGTTGGTTTGATTTTGTCTTACGTCTTGCAAA |
| | TTGCCGGTCAGTTGTCTATGTTGATTAGAACTTTCACCCAGGTCGAAAACGAGATGA |
| | ATTCTGCTGAAAGATTGAACTCTTACGCTACCGATTTGCCAGTTGAAGCACCATATG |
| | TTATTACTGAAAACACTCCACCACCAAACTGGCCTACTCAAGGTAACATTACTTTCG |
| | ATCATGTTTCCTTGGCTTACAGACCAGGTTTGCCATTGGTTTTGAAGGACTTGAATTT |
| | CACTGTTAACCCCATCGAAAAGATCGGTATTTGTGGTAGAACTGGTGCTGGTAAAT |
| | CTTCTATTATGACTGCCTTGTACAGGTTGTCCGAATTGGATTCAGGTAGAATCGAAA |
| | TCGATGGTATCGACATTTCCAAGTTGGGTTTGAGAGATTTGAGGTCCAAGTTGTCCA |
| | TTATTCCACAAGATCCAGTTTTGTTCAGAGGTACTATCAGAACTAACCTGGATCCATT |
| | CAACGAACATGAAGATGATAGATTGTGGGATGCTTTGAGAAGAACAGGTTTGATC |
| | GAAGAATCCAGATTGGAGTCTGTTAAGAAGCAATCTAAGTCTACTACTAAGCCAGC |
| | TACCGGTGAAATTTCTGAAAAGACTACTGAAAAAGCTACTGCTACCCCAGATTCTTT |
| | GGCATTGCATAAGTTTCATTTGGATCAAGCCGTTGAGGATGATGGTTCTAATTTTAG |
| | CTTAGGTGAGAGACAGTTGATCGCTTTTGCTAGAGCTTTGGTTAGAGACTCCAAGA |
| | TTTTGATATTGGATGAAGCCACCTCCTCCGTTGATTACGAAACTGATTCTAAGATCC |
| | AAAAGACGATCATCAGGGAATTCAAGGATTGCACCATTTTGTGCATTGCCCATAGA |
| | TTGAAAACCATCTTGAACTACGACAGGATCTTGGTTTTGGATAAGGGTGAAGTCAA |
| | AGAATTCGATACTCCATGGAATTTGTTCAACGCCAGAGGTTCTATTTTCCAACAAAT |
| | GTGCGAAAGATCCAACGTTACCGAACAAGACTTTGCTTCTTCCGTTCAATTCTAA |
| SEQ ID | MASSSALESNQLSLERQKRLLSFLMSKNVPPVPTQEERKPYPGATANVIKQIFFWWLAP |
| NO: 968 | VMNTGYQRTLQPDDMFYLTDDIKVQKMADDFYRYMSNDIDRSKQKHIAAKCKERGE |
| | TVETTSVDPEEDLKDYELSKFLTVWALAKTFKWQYTWACVCLALSNAGQTTMPLLSKK |
| | LIKYVELKAYGQEPGIGKGLGYSFGTTAMVFIVGVLINHFFYRSMITGAQAKAVLTKALL |
| | DKSFKLSAEAKHKYSVGKITSMLGTDLSRIDFALGFQPFIIVFPIPIAIAIAILIVNIGVASLVG |
| | VGILLVFMVGIAFSTGKLIAYRKKANHYTDSRVNYIKEALNNLKVIKFYSWEPPYHENISD |
| | LRKKEMKIIYRMQVLRNVVTSFAMSLTLFASMTAFLVLYAISNGKRDPASIFSSLSLYNTL |
| | TQQVFLLPMALATGADAFMGISRVGDFMSQSEINPEENAIEAPPDVQEWMDKESLSI |
| | DVEDASFEWEIFEDDEEKDGKNSKKNGSKKRKSEENIILDVQDSESSLTKGLDGSSSSEV |
| | SSAEDAHFEGLNNIDFKIKKGEFVVITGLIGSGKSSLLSAMSGFMKRTSGSVNVNGSLLLC |
| | GYPWVQNSTVKDNIIFGSEFDEEKYKKVIYACSLEADLELLPAGDRTEIGERGITLSGGQK |
| | ARINLARAVYANKDIILLDDVLSAVDARVGKHIMNNCLLDLLKEKTRVLATHQLSLIGSA |
| | DRVIFLNGDGSIDVGTFEELKERNPGFDKLMAYNSESHEEDEEEEENLEEIAEEDLFEDD |
| | KRVVERQLTRRSTRTVTSVGAFPEEEYDEADEEARHHEYNLDEQADGKLIGDEERAVNA |
| | ISKEIYARYFELGSGRLTPWVMLPLLLFFIVVATFSQIFTNTWLSFWTEYKFDKPDKFYIGI |
| | YIMFAFLSFILLTIEFIILVKITNTASVMMNVLAVKKVLHAPMSFMDTTPMGRILNRFTKD |
| | TDVLDNEIGDQLRFFLYVFANIIGVVILCIIYLPWFAIAVPFLGMLFVSISNYYQASAREIKR |
| | LEAVQRSLVYNNFNETLSGMATIKAYKATERFIGKNNYLIDRMNEAYYITIANQRWLAIH |
| | MDFVATLFALLIALLCVNRVFNVSASAVGLILSYVLQIAGQLSMLIRTFTQVENEMNSAE |
| | RLNSYATDLPVEAPYVITENTPPPNWPTQGNITFDHVSLAYRPGLPLVLKDLNFTVNPIE |
| | KIGICGRTGAGKSSIMTALYRLSELDSGRIEIDGIDISKLGLRDLRSKLSIIPQDPVLFRGTIR |
| | TNLDPFNEHEDDRLWDALRRTGLIEESRLESVKKQSKSTTKPATGEISEKTTEKATATPDS |
| | LALHKFHLDQAVEDDGSNFSLGERQLIAFARALVRDSKILILDEATSSVDYETDSKIQKTII |
| | REFKDCTILCIAHRLKTILNYDRILVLDKGEVKEFDTPWNLFNARGSIFQQMCERSNVTE |
| | QDFASSVQF* |
| SEQ ID | ATGGACGACTCCTCTTTGGAAATTGGTAACGACTTGTACAAACCACAGAAGAGAAT |
| NO: 969 | TCTGACCTTCCTGTTCAGAAACAAGTTCTACCCAATTCCAAAGGACGACTCTGAAAG |
| | AAAAATCTACCCAGAGCAATCCTCCAACATCTTGTACAGAGTTTTCTTTTGGTGGGT |
| | GTCTCCATTGATGACTATTGGTTACACTAGAACCTTGCAACCAGATGATTTGTGGAT |
| | TTTGACCGAAGATATGAAGGTCGAACACTTCTACAACTACTTCGTTACTTACTTGCA |
| | GGTTGAAACTGAAAGAGCACATTTGGCTCATATTGCCAACAAGTGCAAAGAAAGAA |
| | ACGAATCCGTTGAGGACTCTTCCAAATCTAGAGAAGAAGATCTGCAAGACTTCGTC |
| | TTGTCTCCAATGAATATTGCTACCGTTTTGTTCTTGACCTTCAAGAGACAAATCTTGG |
| | TCGGTTTGATTTTGGCCATCTTTTCCTTGTCTGGTATTGCTTGTTCTCCACTGTTGACC |
| | AAAGAATTGATTAAGTTCGTCGAGAAGAGATCCTTGGGTGTTCATACAAATATTGG |
| | TCAAGGTATCGGTTACTCTTTGGGTGTTGTTTTCATGATGCTGTTCTCCAACTTGTTG |
| | TTCAACCACTTCATGTACATCGGTCAATCTATGGGTGCTTTGATTAAGGCTTTGTTGA |
| | CTAAGGCTGTTTTGAACAAGGCTTTCAAGTTCAACGCTGAATCCAGACATAAGTTCC |
| | CACAATCTAAGTTGACCTCCATTATTACCACCGATTTGTCCAGAGTTGAAATCGCTGT |
| | TATGTTCCAACCTTTGTTGTTGTGTTTGCCAATTCCTATTGCTATCGCCATCGTTATCT |
| | TGGTTGTTAACATTAGAGTTTCCGCCGTTATCGGTATTGTCATCTTCATTATTTTCTTG |
| | GGCTTCATCTCCATTGGTGCCAAAAAGTTGTTTGCTTACAGAGATGCCGTTTCCAAG |
| | ATTACTGATAAGAGGGTCAATTTCATGAAGGAAATCCTGAACAACCTGAAGATGAT |
| | CAAGTTTTACTCTTGGGAACATCCATACCACGAAAACGTATGTAGAGTTAGAGGTG |
| | AAGAGGTTGACATGATCTTGAAGATTCAAACCTTGAGGAACGTGATTTTCTCTTTGG |
| | CTATGACTTTGACCGGTATCTGTTCTATGATTGCTTTCGTCATCTTGTATGCTATCCA |
| | GGGTTCTACATCTTCTCCAGCTAAAATGTTTTCCTCCGTTTCCACCTTTGAAATCTTG |
| | GGTTTGATGGTTTTCTTCATCCCACAAGCTTTGTCTACTACTGCCGATATGATTAACG |
| | GTTTCAAGAGAATTGGTGCTGTTTTGTCTGCTGATGAAGAGGAACCATACGAAGGT |
| | TACAGAGAATTGAATGATGCTTCCGATAAGAGAGCTATTGCTTTGAAAGATGCCTCT |
| | TTCTCTTGGGATGTTTTCGATGACGAAGAAGGCGAAGAGGACGAAGCTGATGGTG |
| | ACGGTGATGGTGATGAAGAAGAAGAAATTGAAGATAAGAAGGACAAGAAAAAGG |
| | CCAAGAAAGAGAAGAAGCGTAAAGAGAAAGGTAAGGACACTAAGTCTAGTTTCCC |
| | ATCTACCTCTTCCAACGATATTGAGTTGTCTACCATTCCAAAGACCTCTCCAAATTCT |
| | AAGGCTAACAACAAGGGTGAACAAGAAGAAGCTACTTCATTTCCAGGTTTGAAGAA |
| | CATCGATTTGACCATTCATAAGGGTGAGTTCATCGTTATTACTGGTTTGGTTGGTTC |
| | CGGTAAGTCATCTTTGTTATCTGCTATTGCTGGTTTCATGTCTTGCGATTCTGGTGAA |
| | GTTGATATTAACGGTCCATTGCTATTGTGTGGTGCTCCATGGATTCAAAACAATACC |
| | ATCAGAGAGAACATCGTTTTCGGTAAGCCATTCGACCAAGAATATTACGATAAGGT |
| | TATCTACGCTTGCGCCTTGAACATTGATTTGGATTCTTTGGAAGGTGGTGATTACAC |
| | TGAAGTTGGTGAAAAGGGTATTACTTTGTCTGGTGGTCAAAAGGCTAGAATCAATT |
| | TGGCTAGAGCTGTTTACGCCAACAAAGAAATCATCTTGATGGACGATGTTTTGTCAG |
| | CAGTTGATGCTAGAGTTGGTAAGCACATTCTAAACAACTGCTTTTTGGGTTTGTTGG |
| | GCTCTAAGACTAGAGTTTTGGCTACTCATCAATTGTCCTTGATTGGTTCTGCTGACA |
| | GAATCGTTTTCTTGAATGGTGATGGTTCTATCGATGTTGGTAAGATGGATGAATTGA |
| | TCGCCAGAAACAACGACTTTAACCAGTTGATGAAGTTCTCTAAGTTGGAGGACATT |
| | GAGGAAGAAAACTTGGACGTTGAAGTTGAAGAAATCGTTGACGAAATTGACTTGG |
| | GCAACAAAGAATCTAAGGACTCTGGTGAAATCGTGTCCGTTTTCTCTCAAAATCAAT |
| | CCGGTATTGACACCTCTCAAGAATTGACTAGACGTAGAACCAGAATCTTCTCAAAGT |
| | CCGATTCTGACAACAACGACAGCATGGAAGATAATGATGTTGAGTACAAGGACTAC |
| | AACCATAACAAGGATGCTACCAAGGGTAAGATTATCACAGAAGAAGAGAGAGCCG |
| | TTAACTCCATCAAGTTTGATGTTTACCACAAGTACTTAAAGTACGGTGCTGGTAAAT |
| | TGACTCCATGGGGTTTCTTTACTATTTTCGCTGTCTTGTTGACTTTGGCTACCTTCTGT |
| | GACATTTTCACTAACACTTGGTTGTCTTTCTGGATCGAACAAAAGTTCGACGGTAAA |
| | TCCAACGGTTTCTACATTGGTTTCTACGTCATGTTCAATGTCTTGTGGGTTATCTTCTT |
| | GACTTACACCTTCGTGTTCTTCATTCATGGTACTACCGTTTCTTCTAAGCACTTGAATT |
| | TGATGGCCATTAAGAGAATCTTGCATGCCCCAATGTCTTTCATGGACACTACTCCAA |
| | TGGGTAGAATTTTGAACAGATTCACCAAGGATACCGATGCCTTGGATAACGAAATT |
| | TCTGATAACCTGAGGTTGTTCTTCACCGCTATTGCTAAAATGATCGGCGTGTTCATTC |
| | TGATCATCATCTATTTGCCATGGTTCGCTTGTGCTATTCCAGGTATTTTCGTGCTGTT |
| | CTTCTTGATTGCTAACTTCTACCAAGCCTCTAACAGAGAAGTTAAGAGATTGGAAGC |
| | CATCTTGAGATCCTTCGTTTACAACAACGTTAACGAAGTCCTATCCGGTATGAATAC |
| | CATTAAGGCCTATAAGGATGAATCCAGGTTCGCTGATTTGGGTGATTTGTTATTGAA |
| | CAAAGCTAACGAAGCCTCCTTCGTTGTTAACGCTAATCAAAAATGGTTGGGTATCCA |
| | GTTGGATTTGTTGGCTGAATTGATTGTCTTGATCGTGTCCTTGTTGTGTGTTAACAG |
| | AGTGTTCTCTATTAACGCTGCTGCTGTTGGTTTGTTGATGACTTATACTTTACAAGTC |
| | GCCAACGAGCTGTTGAATTTGGTTAGAACTTTCACCTTGGTGGAAAACGATATGAA |
| | CTCCGCCGAAAGAATTATTCATTACGCCTTGAAAGTTGAACAAGAGGCCCCATACAA |
| | AATCGATTCTTCTCAACCACCACCAGATTGGCCACAATATGGTGCAGTTGATTTCAA |
| | GAACGTCAACATGAAGTATAGACCAGGTTTGCCATTGGTTTTGAAGGACTTCTCATT |
| | GAAGATTGCCCCTATGGAAAAGATTGGTATTTGTGGTAGAACTGGTGCCGGTAAAT |
| | CTTCTATTATGACTGCCTTGTATAGGATCTCCGAATTGGATTCAGGTTCCATCAGAAT |
| | TGATGACGTTGATATTGCCACTATCGGCTTGAAAGATTTGAGATCCCATTTGTCCAT |
| | CATTCCACAAGATCCAGTTTTGTTCAACGGCACTATCAGATCTAATTTGGATCCATTT |
| | GGTGAACAACCAGACGACGTTTTGTGGGAATCTTTAAGAAGATCAGGTATCTTGAC |
| | CACCGAAGAAGTTGCTAAAGCTAGAGCTATTTCCAAGGATTCTATTACTGCTGCTTC |
| | TGGTGGTAAAGAAGGTTCAGAAGTTGAATCACAAGAAGTCGAATTGCCAAAGTTCC |
| | ACTTGTATCAACCTGTTGAAGATGAAGGTGAGAATTTCTCATTGGGTGAAAGACAG |
| | TTGATCTCTTTTGCTAGAGCTTTGGTCAGAAACGCCAAGATCATTATATTGGATGAA |
| | GCCACCTCTTCCGTTGATTATGGTACTGATGACAAAATCCAAACCACTATCGCACAA |
| | GAATTCAAGTCCTGTACCATTTTGTGCATTGCCCACAGATTGAAAACGATCATCAAC |
| | TACGATAAGATCCTGGTTATGGATAAGGGTTCCGTTAGAGAATTTGATACTCCATG |
| | GAACCTATTCAACTCAAACGGTTCTGTTTTCAGGGAAATGTGCGAAAAGTCTAACAT |
| | AGTTGCTGAGGACTTTAAGAGGCGTTGA |
| SEQ ID | MDDSSLEIGNDLYKPQKRILTELFRNKFYPIPKDDSERKIYPEQSSNILYRVFFWWVSPLM |
| NO: 970 | TIGYTRTLQPDDLWILTEDMKVEHFYNYFVTYLQVETERAHLAHIANKCKERNESVEDSS |
| | KSREEDLQDFVLSPMNIATVLFLTFKRQILVGLILAIFSLSGIACSPLLTKELIKFVEKRSLGV |
| | HTNIGQGIGYSLGVVFMMLFSNLLFNHFMYIGQSMGALIKALLTKAVLNKAFKFNAESR |
| | HKFPQSKLTSIITTDLSRVEIAVMFQPLLLCLPIPIAIAIVILVVNIRVSAVIGIVIFIIFLGFISIG |
| | AKKLFAYRDAVSKITDKRVNFMKEILNNLKMIKFYSWEHPYHENVCRVRGEEVDMILKI |
| | QTLRNVIFSLAMTLTGICSMIAFVILYAIQGSTSSPAKMFSSVSTFEILGLMVFFIPQALST |
| | TADMINGFKRIGAVLSADEEEPYEGYRELNDASDKRAIALKDASFSWDVFDDEEGEEDE |
| | ADGDGDGDEEEEIEDKKDKKKAKKEKKRKEKGKDTKSSFPSTSSNDIELSTIPKTSPNSKA |
| | NNKGEQEEATSFPGLKNIDLTIHKGEFIVITGLVGSGKSSLLSAIAGFMSCDSGEVDINGP |
| | LLLCGAPWIQNNTIRENIVFGKPFDQEYYDKVIYACALNIDLDSLEGGDYTEVGEKGITLS |
| | GGQKARINLARAVYANKEIILMDDVLSAVDARVGKHILNNCFLGLLGSKTRVLATHQLSL |
| | IGSADRIVFLNGDGSIDVGKMDELIARNNDFNQLMKFSKLEDIEEENLDVEVEEIVDEID |
| | LGNKESKDSGEIVSVFSQNQSGIDTSQELTRRRTRIFSKSDSDNNDSMEDNDVEYKDYN |
| | HNKDATKGKIITEEERAVNSIKFDVYHKYLKYGAGKLTPWGFFTIFAVLLTLATFCDIFTNT |
| | WLSFWIEQKFDGKSNGFYIGFYVMFNVLWVIFLTYTFVFFIHGTTVSSKHLNLMAIKRIL |
| | HAPMSFMDTTPMGRILNRFTKDTDALDNEISDNLRLFFTAIAKMIGVFILIIIYLPWFACA |
| | IPGIFVLFFLIANFYQASNREVKRLEAILRSFVYNNVNEVLSGMNTIKAYKDESRFADLGD |
| | LLLNKANEASFVVNANQKWLGIQLDLLAELIVLIVSLLCVNRVFSINAAAVGLLMTYTLQ |
| | VANELLNLVRTFTLVENDMNSAERIIHYALKVEQEAPYKIDSSQPPPDWPQYGAVDFKN |
| | VNMKYRPGLPLVLKDFSLKIAPMEKIGICGRTGAGKSSIMTALYRISELDSGSIRIDDVDIA |
| | TIGLKDLRSHLSIIPQDPVLFNGTIRSNLDPFGEQPDDVLWESLRRSGILTTEEVAKARAIS |
| | KDSITAASGGKEGSEVESQEVELPKFHLYQPVEDEGENFSLGERQLISFARALVRNAKIIIL |
| | DEATSSVDYGTDDKIQTTIAQEFKSCTILCIAHRLKTIINYDKILVMDKGSVREFDTPWNL |
| | FNSNGSVFREMCEKSNIVAEDFKRR* |
| SEQ ID | ATGTCCACCACCTCCATCATTTCTGAAAAGGGTCATAACTCTAGAGACTC |
| NO: 971 | CGGTGTTGAAAACGATATTAGAGATTTGGCTAGAGCTTTCACTAACGCCT |
| | CTTCTATTACTTATCCAATGACTGGTGGTGACACCTCTTCATTGAATTCTA |
| | AAGCTCCAGTTAACCCAGTCTTGACCAATTACGAATCCGATGATTACATCC |
| | CAAAGTTGGACCCAAATTCCGATGAATTCTCATCCATTGAATGGGTCAGA |
| | AACCTGTCCAAGTTGATTTTGAATGCTCCAGAACATTACAAGCCATACACT |
| | TTGGGTTGTACTTGGAAGAATTTGAGAGCTTTTGGTTCCTCTGCTGATGTT |
| | GCTTATCAATCTACTGTTGCCAACATTCCAAAGAAGTTGTGCGAATTCTTC |
| | TACAGAAAGTGCCATAAGGCTAACGAGGATAACAACATCGATATCTTGAA |
| | ACCTATGGACGGTTTGATTGAACCAGGTGAATTATTGGTTGTTTTGGGTA |
| | GACCAGGTTCTGGTTGTACTACTTTGTTGAAGTCCATTTCCTCTAACACTC |
| | ACGGTTTCAAGTTGGACTCTAACTCTATCGTTGAATACGATGGTATTTCCC |
| | CAGAAGAGATCAAAAAGCACTATAGAGGTGAAGTTGTTTACAACGCTGAA |
| | GCCGATGTTCATTTTCCACATTTGACTGTTTTCGAAACCTTGAACACCATT |
| | GCTTTGTTGTCTACTCCATCCAATAGAATCCCAGGTGTTTCTAGAACTGCT |
| | TTTGCTAAACATTTGACCGAAGTTGTTATGGCTACTTACGGTTTGTTGCAT |
| | ACCAGAAACACTAAGGTTGGTAACGAATTGGTTAGAGGTGTTTCAGGTGG |
| | TGAAAGAAAGAGAGTTTCTATTGCCGAAGTTTCCATTTGCGGTTCTAAATT |
| | GCAATGTTGGGATAACGCTACTAGAGGTTTGGATTCTGCTACTGCTATGG |
| | AATTTGTTAAGGCTTTGAGAACCTCTGCCAGAATGATGAAGTCATCTTCTG |
| | CTGTTGCTATCTACCAATGTTCTCAAGAAACCTACAACTTGTTCGATAAGG |
| | TTTGCGTCTTGTACGAAGGTAGACAAATCTATTTCGGTTCTGCTAACGAAG |
| | CCAAGCAATACTTTGAAGAGTTGGGTTACATTTGCCCAGAAAGACAAACT |
| | ACCGCTGATTTCATTACTGCTGTTACTTCTCCAGGTGAAAGGATTGCAAAC |
| | GAAAACAAGAAGTTCGTTCCATCCACTGCTGAAGAAATGGAAAAACATTG |
| | GAAGAACTCCGAACAGTACAGACACTTGTTGTCCAAAATTGAAAAGAGAC |
| | AGAACGAGGACAACTCCGGTAAAAAGACTGATTTGAGAAAAGGTCATGTC |
| | GCCAGACAATCTCATAGAGCTAGAGCATCTTCTCCATTCATCGTTTCTTAT |
| | TGGCTGCAGGTTAAGTACTTGTTGGAAAGAAACTTCCAGAGGATCAGAAA |
| | CTCTATTGGTTTGACCTTGTTCTTGGTCTTGGGTAACTCTTCTATGTCTTT |
| | GTTGTTGGGCTCTATGTTCTACAAGGTTTTGAAGCACGATAATACTGCTG |
| | GCTTGTATTCTAGAGCTGCTGCTTTGTTTTTCGCCGTTTTGTTTAATGCTTT |
| | CAGCTGCATGTTGGAAGTTTTGGCCTTGTATGAATCCAGACCAATCATTG |
| | AAAAGCACAAGAGGTACAGCTTGTATCATCCTTCAGCTGATGCTTTGGCC |
| | TCTATTATTAGTGAAATCCCATCTAAGTTGGTCACCGCTGTTTTCTTTAACA |
| | TCGTGTTCTACTTTCTGTGCAACTTCAAAACTGATGCTGGTGCTTTCTTCT |
| | TCTACTTCATGATGTCTTTGATTGCCACCTTCGTTATGTCCCATATTTTCAG |
| | ATGTATTGGTTCCGCTACTAAGACTTTGGCTCAAGCTATGGTTCCAGGTT |
| | CAGTTTTGTTGTTAGCTATGTCTATCTACACCGGTTTCGCTATCCCAAAGA |
| | CTAAGATTTTGAGATGGTCTAAGTGGATCTGGTACATTAACCCATTGGCCT |
| | ACATTTTCGAATCCATGATGGTTAACGAATTCCACGATCATAACTTCGAAT |
| | GCTCTGAGTATATTCCAAGAGGTCCAGGTTACCATAACATCTCTGGTACT |
| | GAAAGAGTCTGTTCTTCTATTGGTGCTAAGCCTGGTGAAAATTTTGTTGAT |
| | GGTGAGTTGTACATCAACGCCTCATATGGTTATTATCATGGTCACAAATG |
| | GCGTGGTTTCGGTATTGGTTTAGCTTACGCTATTTTCTTCTTGGGCCTGTA |
| | CTTGGTTATTACCGAATTCAACGAATCCGCTAAGCAAAAGGGTGAAATCTT |
| | GGTTTTTAGACAGTCCACCTTGAAGAACAACACTAAGAAAACCAGGTACA |
| | TCTCCGATTTGGAATCTGGTGGTGGTGCTGCTTCTACATCAGAAAAGAAT |
| | TTGGTTGATGACTCTGGTGATAACGGTTTGGGTTCCATTAGACAGATCGA |
| | ATTGTCTAAGTCCGAAGCCATTTTTCATTGGAGAAACGTTTGTTACGATGT |
| | CGTTGTCAAAGGTGAAACTAAGAGAATCTTGAACGGTATTGATGGTTGGG |
| | TTAAGCCAGGTACTTTGACTGCTTTGATGGGTGCTTCTGGTGCTGGTAAA |
| | ACTACTTTATTGGATTGCTTAGCTTCCAGAGTTACCTCTGGTGTTATTACT |
| | GGTGATATCTTCGTTAACGGTCACTTGAGAGATTCTTCATTCGCTAGATCA |
| | ATTGGTTACTGTCAACAACAAGACTTGCATTTGGAAACTGCTACCGTTAGA |
| | GAATCCTTGAGATTTGCTGCTTATTTGAGACAGCCAAGATCCGTTAGTATC |
| | GAATCTAAGAACAGATACGTCGAGTCCGTTATCAACATCTTGGAAATGAA |
| | GCAATACGCTGATGCTATCGTTGGTGTTAGTGGTGAAGGTTTGAATGTCG |
| | AACAGAGAAAAAGATTGACCATCGGTGTAGAATTGGCTGCTAAACCTAAG |
| | TTGTTGGTCTTTTTGGATGAACCTACTTCTGGTTTGGATAGTCAAACTGCT |
| | TGGTCTATTTGCCAGTTGATGAGAAAATTGGCTGATCACGGTCAAGCTGT |
| | TTTGTGTACTATTCACCAACCTTCCGCTTTGCTGTTGCAAGAATTCGATAG |
| | GTTGTTGTTCCTACAAAGAGGTGGTCAAACAGTTTACTTTGGTGATTTGG |
| | GTGAAAGATGCCAAACCATGATTAGGTACTTCGAAAAGAATGGTGCTCAT |
| | CAATGTCCAAAGGATGTTAATCCAGCAGAATGGATGTTAGAAGTTATTGG |
| | TGCAGCTCCAGGTTCTCATGCTGATCAAGATTACCATGAAGTTTGGAAGG |
| | GTTCTGAAGAATGTACTGCTACTCAAGCTGAATTGGAATGGATGGAAAAA |
| | GAATTGGGTAAGAAGCCACAAGACAACTTGGAAAGAGGTGAATTTGCTTC |
| | CTCACTGTTGTCCCAATACTTTTTGGTTACTAAGAGGCTGTTCCAACAGTA |
| | TTGGAGAACACCATCTTACTTGTGGTCTAAGGCTATCTTGACTTTGTTCTC |
| | CCAAATCTTCATCGGTTTCACTTTCTTTAAGGCCGACAGATCATTGCAAGG |
| | TCTGCAAAATCAAATGCTGTCCGTTTTCATGTTCACCGTTGTTTTTAATCCT |
| | GCCGTGCAACAATATTTGCCAACCTATATTTCTCAGAGGGACTTATACGAA |
| | GCTAGAGAAAGACCATCTAAGACCTTTTCATGGATTGCCTTCATTTTGTCC |
| | CAAATTACCGTTGAAATTCCTTGGAACTTCGGCATTGGTACATTGGGTTTC |
| | TTGTGTTACTATTACCCAGTCTCTTTCTACCGTAATGCTTCTTTTGCCAATC |
| | AATTGCATGAGAGAGGTGCTTTGTTCTGGTTATTCTGTACAGCTTTCTACG |
| | TTTTCACTGGTTCTATGGCTCAATTGTGTGTTGCTGGTCAAGAAGTTGCTC |
| | AATCTGCTGGTCATATTGCTAGCTTGTTGTTTGTCTTGTCCTTGTCTTTTTG |
| | CGGTGTTATGGTTGCTCCAAAGAATATGCCAGGTTTTTGGAAGTTCATGT |
| | ACAGAGTTTCTCCACTGACTTACTTCATCGATGGTGTTTTGTCTACTGGTA |
| | TTGCCAACTCTAAGGTTGAATGTTCCGATTACGAATTTGTCACTTTCACTC |
| | CAAGATCTGGTCAAACTTGTGGTGAGTATATGTCCTTGTATATTGATGCTG |
| | CTGGTACTGGTTACATGAAGGATTCTGATTCTACCACCAAGTGTTCTTTTT |
| | GTCCAGCTTCTTCCACTAACGTGTTCTTGAAAATGGTGTCCTCTAATTACT |
| | CTCACAGATGGCGTAACTACGGTATCTTTTTGTGCTACATTTGCTTCAACG |
| | TTTTCGCTGCAGTTTTCTTGTACTGGTTGGCAAGAGTTCCTAAAAGAAAGG |
| | CTATGGTTACCGATAAGAGAAAGCCAACTGCTAAGAAGTGA |
| SEQ ID | MSTTSIISEKGHNSRDSGVENDIRDLARAFTNASSITYPMTGGDTSSLNSKAPVNPVLTN |
| NO: 972 | YESDDYIPKLDPNSDEFSSIEWVRNLSKLILNAPEHYKPYTLGCTWKNLRAFGSSADVAY |
| | QSTVANIPKKLCEFFYRKCHKANEDNNIDILKPMDGLIEPGELLVVLGRPGSGCTTLLKSI |
| | SSNTHGFKLDSNSIVEYDGISPEEIKKHYRGEVVYNAEADVHFPHLTVFETLNTIALLSTPS |
| | NRIPGVSRTAFAKHLTEVVMATYGLLHTRNTKVGNELVRGVSGGERKRVSIAEVSICGS |
| | KLQCWDNATRGLDSATAMEFVKALRTSARMMKSSSAVAIYQCSQETYNLFDKVCVLY |
| | EGRQIYFGSANEAKQYFEELGYICPERQTTADFITAVTSPGERIANENKKFVPSTAEEME |
| | KHWKNSEQYRHLLSKIEKRQNEDNSGKKTDLRKGHVARQSHRARASSPFIVSYWLQVK |
| | YLLERNFQRIRNSIGLTLFLVLGNSSMSLLLGSMFYKVLKHDNTAGLYSRAAALFFAVLFN |
| | AFSCMLEVLALYESRPIIEKHKRYSLYHPSADALASIISEIPSKLVTAVFFNIVFYFLCNFKTD |
| | AGAFFFYFMMSLIATFVMSHIFRCIGSATKTLAQAMVPGSVLLLAMSIYTGFAIPKTKILR |
| | WSKWIWYINPLAYIFESMMVNEFHDHNFECSEYIPRGPGYHNISGTERVCSSIGAKPGE |
| | NFVDGELYINASYGYYHGHKWRGFGIGLAYAIFFLGLYLVITEFNESAKQKGEILVFRQST |
| | LKNNTKKTRYISDLESGGGAASTSEKNLVDDSGDNGLGSIRQIELSKSEAIFHWRNVCYD |
| | VVVKGETKRILNGIDGWVKPGTLTALMGASGAGKTTLLDCLASRVTSGVITGDIFVNGH |
| | LRDSSFARSIGYCQQQDLHLETATVRESLRFAAYLRQPRSVSIESKNRYVESVINILEMKQ |
| | YADAIVGVSGEGLNVEQRKRLTIGVELAAKPKLLVFLDEPTSGLDSQTAWSICQLMRKL |
| | ADHGQAVLCTIHQPSALLLQEFDRLLFLQRGGQTVYFGDLGERCQTMIRYFEKNGAHQ |
| | CPKDVNPAEWMLEVIGAAPGSHADQDYHEVWKGSEECTATQAELEWMEKELGKKP |
| | QDNLERGEFASSLLSQYFLVTKRLFQQYWRTPSYLWSKAILTLFSQIFIGFTFFKADRSLQ |
| | GLQNQMLSVFMFTVVFNPAVQQYLPTYISQRDLYEARERPSKTFSWIAFILSQITVEIPW |
| | NFAIGTLGFLCYYYPVSFYRNASFANQLHERGALFWLFCTAFYVFTGSMAQLCVAGQEV |
| | AQSAGHIASLLFVLSLSFCGVMVAPKNMPGFWKFMYRVSPLTYFIDGVLSTGIANSKVE |
| | CSDYEFVTFTPRSGQTCGEYMSLYIDAAGTGYMKDSDSTTKCSFCPASSTNVFLKMVSS |
| | NYSHRWRNYGIFLCYICFNVFAAVFLYWLARVPKRKAMVTDKRKPTAKK- |
| SEQ ID | ATGGAATTCGAGCCAAAGTCCGAAGGTTCTTTGCCATCTTATGAAGGTTT |
| NO: 973 | GGATAAGTCCGCTGAAGTTCAAGTTCAAAGATTGGCTCATGGTGTTAACC |
| | CAATTCATGAAGGTGCTCCACAATTGGATCCTAACTCTTCTGATTTTTCAT |
| | CCAAAGCCTGGATTCAGAATATGGCCAATTTGTCATCTGAAGATCCAGAT |
| | CATTTCAAGCCATACCAAGTTGGTTGTTGTTGGAAAGATTTGGCTGCTTCT |
| | GGTGCTTCTGCTGATGTTGCTTATCAAACTACTGTTGAAAACTTGCCATGG |
| | AAGGTTTTGTTCTGGATCTATAGAAAATTGAGGCCCACCAGAAAGTCCGA |
| | TATTTTCCAAATTTTGAAGCCAATGGATGGTGCTTTGGATCCAGGTGAAGT |
| | TTTGGTTGTTTTAGGTAGACCAGGTTCTGGTTGTACTACTTTGTTGAAGTC |
| | TATTGCCTCTAACACCCACGGTTTTAACATTGCTAAGGATTCCACCATTTC |
| | CTACTCTGGTTTGTCTCCAAAGGATATCAACAGACATTTTAGAGGTGAAGT |
| | CGTTTACAACGCCGAAACCGATATTCATTTGCCACATTTGACTGTCTACCA |
| | AACCTTGTTGACTGTGTCTAGATTGAAAACTCCACAGAACAGAATCAAGG |
| | GTGTTGATAGAGAAACTTGGGCTAGACATATGACCGATGTTGTTATGGCT |
| | ACTTATGGTTTGTCCCATACCAAAAACACAAAGGTTGGTGGTGATTTGGTT |
| | AGAGGTGTTTCAGGTGGTGAAAGAAAGAGAGTTTCTATTGCCGAAGTTAC |
| | CATTTGCGGTTCTAAGTTTCAATGTTGGGATAACGCTACCAGAGGTTTAG |
| | ATGCTGCTACTGCTTTGGAATTCATTAAGGCTTTGAGAACCCAAGCTGATA |
| | TTTTGGCTTCTACTGCTTGTATTGCTATCTACCAATGTTCCCAAAACGCCT |
| | ACGATTTGTTCGATAAGGTTTGTGTCTTGTACTCCGGTTACCAAATTTTCT |
| | TTGGTTCTGCTGGTGATGCCAAGAGATACTTTGAAGAAATGGGTTACCAT |
| | TGTCCATCCAGACAAACTACAGCTGATTTCTTGACTTCTGTTACTTCTCCA |
| | GCTGAAAGAACTGTTAACAACGAGTACATTGAAAAGGGTATCCACGTTCC |
| | AGAAACTCCAGAAGAAATGTCTGATTATTGGAGGAACTCTCAAGAGTACA |
| | GAGACTTGCAAGAACAGATCCAAAACAGATTGGATCAGAACCATGAAGAA |
| | GGTTTGAGAGCCATCAAAGAATCTCATAATGCTGCCCAATCTAAGAGGAC |
| | TAGAAGATCATCTCCATACACTGTTTCTTACGGTATGCAGATCAAGTACCT |
| | GTTGATTAGAAATATGTGGCGTATCAAGAACTCCTCCGGTATTACCATTTT |
| | TCAGGTTTTCGGTAATTCCGTCATGGCCTTGTTGTTAGGTTCTATGTTTTA |
| | CAAGGTCCTGAAGCCATCTTCTACTGATACTTTTTACTATAGAGGTGCCG |
| | CTATGTTCTTCGCCATTTTGTTTAATGCTTTCAGCTCCTTGTTGGAGATCTT |
| | CTCATTATATGAAGCCAGACCAATCACCGAAAAGCACAGAACTTATTCCTT |
| | GTATAGACCTTCCGCTGATGCTTTTGCTTCTGTTTTGTCTGAAATTCCACC |
| | AAAGATCGTTACCGCTATTTGTTTTAATGTCGCCTTGTACTTCTTGGTCCA |
| | CTTTAGAGTTGATGCTGGTAGATTCTTCTTCTACTTCCTGATTAACATCCT |
| | GGCCATCTTTTCCATGTCTCATATGTTTAGATGTGTCGGCTCTTTGACTAA |
| | GACTTTGACTGAAGCTATGGTTCCAGCCTCTATCTTGTTATTGGTTTTGTC |
| | TATGTACACGGGTTTCGCTATTCCAAAGACTAAGATGTTAGGTTGGTCTAA |
| | GTGGATCTGGTACATTAACCCATTGTCCTACTTGTTCGAAGCCTTGATGG |
| | TTAACGAATTCCACGACAGAAACTTCTCTTGCACTTCTTTTATTCCAATGG |
| | GTCCAGGTTACCAATCAGTTTCTGGTACTCAAAGAGTTTGCGCTGCAGTT |
| | GGTGCTGAACCAGGTCAAGATTATGTTTTGGGTGATAACTACATCAAGCA |
| | GTCTTACGGTTACGAAAACAAACATAAGTGGCGTGCATTTGGTGTTGGAA |
| | TGGCTTATGTTATCTTTTTCTTCTTCGTGTACCTGTTCTTGTGCGAAGTTAA |
| | TCAAGGTGCTAAGCAAAACGGTGAGATTTTGGTTTTTCCACAATCCGTTGT |
| | CAGAAAGATGAGAAAGCAGAAGAAAATCTCTGCCGGTTCTAACGATTCTT |
| | CTGATCCTGAAAAGACCATCGGTGTTAAGGTTAATGATTTGACTGATACG |
| | ACGCTGATCAAGAATTCTACAGATTCATCCGCCGAACAGAACCAGGATAT |
| | TGGTTTGAACAAATCCGAAGCCATTTTCCATTGGAGAAACGTTTGTTACGA |
| | TGTCCAAATCAAGTCCGAAACCAGAAGAATTTTGGATAACATTGATGGTTG |
| | GGTCAAGCCAGGTACTTTAACTGCTTTGATGGGTGCTACTGGTGCTGGTA |
| | AAACTACTTTATTGGATTCCTTGGCTCAAAGGGTTACTACTGGTGTTTTGA |
| | CTGGTTCCATTTTCGTTGACGGTAAATTGAGAGATGAATCCTTCGCTAGAT |
| | CTATCGGTTACTGTCAACAACAAGACTTGCATTTGACTACTGCTACCGTTA |
| | GAGAAAGCTTGTTGTTTTCAGCTATGTTGAGACAACCTAAGTCTGTTCCAG |
| | CTTCTGAAAAGAGGAAATACGTTGAAGAAGTCATCAACGTCTTGGAGATG |
| | GAACCTTATGCTGATGCTATAGTTGGTGTTGCTGGTGAAGGTTTAAACGT |
| | CGAACAAAGAAAAAGGTTGACCATCGGAGTTGAATTGGCTGCTAAACCTA |
| | ATCTGCTGTTGTTCTTGGATGAACCTACAAGTGGTTTGGATTCTCAAACTG |
| | CTTGGTCTATTTGCCAGTTGATGAAGAAGTTGGCTAATAGAGGTCAAGCT |
| | ATTTTGTGCACCATTCATCAACCATCTGCCATGTTGATCCAAGAATTTGAT |
| | AGGTTGCTGTTCCTGCAAAAAGGTGGTCAAACTGTTTATTTTGGTGACTTG |
| | GGTAAAGACTGCAAGTCCATGATTCATTACTTCGAATCTCACGGTTCTCAT |
| | AAGTGTCCATCTGATGGTAATCCAGCAGAATGGATGTTGGAAATTGTCGG |
| | TGCTGCTCCAGGTACTCATGCTAATCAAGATTACTACGAAGTTTGGCGTA |
| | ACTCCGAAGAATATCAAGAGGTCAGAAAAGAATTGGACAGGATGGAAGAT |
| | GAATTGAAGGGTATTGATGGTGGTGACGAACCAGAAAAACACAGATCTTT |
| | TGCTACTGACATCTTCACCCAGATCAGATTGGTTTCCCATAGATTGCTACA |
| | ACAATATTGGAGGTCACCCTCTTACTTGTTTCCAAAGTTTTTGCTGACAGT |
| | GTTCTCCGAGTTGTTCATCGGTTTTACTTTGTTCAAGGCTGACAGAAGCTT |
| | GCAAGGTCTACAAAATCAAATGCTGTCCGTTTTCATGTACACCGTTGTTTT |
| | TAACACCTTGTTGCAGCAATACTTGCCCTTGTATGTTCAGCAAAGAAACTT |
| | GTACGAAGCTAGAGAAAGACCTTCTAGAACTTTCTCTTGGTTTGCCTTCAT |
| | CGTGTCCCAAATCTTCATTGAAGTTCCTTGGAACATTTTGGCTGGTACTGT |
| | TGCTTTCTTTTGTTACTATTACCCAATCGGCTTCTACAGAAACGCTTCTGA |
| | ATCTCATCAATTGCACGAAAGAGGTGCTTTGTTTTGGTTATTCTCTACCGC |
| | TTACTACGTCTGGATTGGTTCTATGGGTTTGTTGGCTAACTCTTTCATCGA |
| | ACATGATGTTGCAGCTGCTAATTTGGCATCTTTGTGTTATACTTTGGCCTT |
| | GTCTTTTTGCGGTGTTTTGGCTACTCCAAAAGTTATGCCAAGATTCTGGAT |
| | ATTCATGTACCGTGTTTCTCCCTTGACCTACTTCATTGATGCTACTTTAGC |
| | TACCGGTATTGCTAACGTTGATGTTAAGTGTGCTGATTACGAATTCGCTAA |
| | GTTCACTCCACCAAAAGGTCAAAATTGTGGTGACTACATGAAGAACTTCAT |
| | TAAGTCTGCTGGTACGGGTTACTTGAAAGATTCTTCAGCTGTTGACGAAT |
| | GCAACTTCTGCCAATTCTCTACTACTAATGCCTACTTGGAATCCGTTACCT |
| | CTTCATACTCTAGACGTTGGAGAAATTACGGTATCTTCATTTGCTTCATTG |
| | CCTTCGATTATATTGCCGCCGTTTTCTTATATTGGTTGGCTAGAGTTCCAA |
| | AGAAGTCCGGTAAAGTTTCAGGTAAGAAGTAA |
| SEQ ID | MEFEPKSEGSLPSYEGLDKSAEVQVQRLAHGVNPIHEGAPQLDPNSSDFSS |
| NO: 974 | KAWIQNMANLSSEDPDHFKPYQVGCCWKDLAASGASADVAYQTTVENLPW |
| | KVLFWIYRKLRPTRKSDIFQILKPMDGALDPGEVLVVLGRPGSGCTTLLKSIAS |
| | NTHGFNIAKDSTISYSGLSPKDINRHFRGEVVYNAETDIHLPHLTVYQTLLTVS |
| | RLKTPQNRIKGVDRETWARHMTDVVMATYGLSHTKNTKVGGDLVRGVSGG |
| | ERKRVSIAEVTICGSKFQCWDNATRGLDAATALEFIKALRTQADILASTACIAIY |
| | QCSQNAYDLFDKVCVLYSGYQIFFGSAGDAKRYFEEMGYHCPSRQTTADFL |
| | TSVTSPAERTVNNEYIEKGIHVPETPEEMSDYWRNSQEYRDLQEQIQNRLDQ |
| | NHEEGLRAIKESHNAAQSKRTRRSSPYTVSYGMQIKYLLIRNMWRIKNSSGIT |
| | IFQVFGNSVMALLLGSMFYKVLKPSSTDTFYYRGAAMFFAILFNAFSSLLEIFS |
| | LYEARPITEKHRTYSLYRPSADAFASVLSEIPPKIVTAICFNVALYFLVHFRVDA |
| | GRFFFYFLINILAIFSMSHMFRCVGSLTKTLTEAMVPASILLLVLSMYTGFAIPK |
| | TKMLGWSKWIWYINPLSYLFEALMVNEFHDRNFSCTSFIPMGPGYQSVSGT |
| | QRVCAAVGAEPGQDYVLGDNYIKQSYGYENKHKWRAFGVGMAYVIFFFFVY |
| | LFLCEVNQGAKQNGEILVFPQSVVRKMRKQKKISAGSNDSSDPEKTIGVKVN |
| | DLTDTTLIKNSTDSSAEQNQDIGLNKSEAIFHWRNVCYDVQIKSETRRILDNID |
| | GWVKPGTLTALMGATGAGKTTLLDSLAQRVTTGVLTGSIFVDGKLRDESFAR |
| | SIGYCQQQDLHLTTATVRESLLFSAMLRQPKSVPASEKRKYVEEVINVLEME |
| | PYADAIVGVAGEGLNVEQRKRLTIGVELAAKPNLLLFLDEPTSGLDSQTAWSI |
| | CQLMKKLANRGQAILCTIHQPSAMLIQEFDRLLFLQKGGQTVYFGDLGKDCK |
| | SMIHYFESHGSHKCPSDGNPAEWMLEIVGAAPGTHANQDYYEVWRNSEEY |
| | QEVRKELDRMEDELKGIDGGDEPEKHRSFATDIFTQIRLVSHRLLQQYWRSP |
| | SYLFPKFLLTVFSELFIGFTLFKADRSLQGLQNQMLSVFMYTVVFNTLLQQYL |
| | PLYVQQRNLYEARERPSRTFSWFAFIVSQIFIEVPWNILAGTVAFFCYYYPIGF |
| | YRNASESHQLHERGALFWLFSTAYYVWIGSMGLLANSFIEHDVAAANLASLC |
| | YTLALSFCGVLATPKVMPRFWIFMYRVSPLTYFIDATLATGIANVDVKCADYE |
| | FAKFTPPKGQNCGDYMKNFIKSAGTGYLKDSSAVDECNFCQFSTTNAYLES |
| | VTSSYSRRWRNYGIFICFIAFDYIAAVFLYWLARVPKKSGKVSGKK- |
| SEQ ID | ATGTCCAACTCCGCCTACGATATTGAAGATCATCCTGAAGAGGTTAACAAGTACGAT |
| NO: 975 | GGTTACAACAACGCTGTTGATTCCGAAGTTCAAAGATTGGCTAGACAAATCACCCA |
| | AAACTCCCAATTGTCTTTTCAAGATGACGGTTTTAAGTTGGCTCCAGGTGAATCTAA |
| | TATCGACGGTTTGTCTAGAGTTTCTACTATTGCTCCTGGTGTTAACCCAATGCAAAAC |
| | GTTGAAGAATTGGACCCAAGATTGGATCCTAACTCTGAAGAATTCCAATCCAGATA |
| | CTGGATCAAGAACTTCAAGGCTTTGATGGATAAGGATCCAGATCACTACAAGAACT |
| | ACTCATTGGGTGTTACCTTCAAGAATTTGAGAGCTTCTGGTGAAGCTTCTGATGCTG |
| | ATTATCAAACCACCATTATTAACGCCCCATTCAAGATTGCTAAGCAATATGCTAAAG |
| | CCGTGTTCTCTACTAGATCTGCTAAACAAGCTAACAGGTTCGACATCTTGAAGTCTTT |
| | GGATGGTATAGTTAGACCAGGTGAAGTTTTGGTTGTTTTGGGTAGACCTGGTTCTG |
| | GTTGTACTACTTACTTGAAATCCATTGCCTCTAACACCCATGGTTTTAAGATAGGTCA |
| | AGAGTCCGAAATGTCTTACGAAGGTTTGACCCAAAAAGAGATCAAAAAGCACTTTA |
| | GAGGTGAGGTTGTTTACAACGCCGAATCCGATATTCATTTCCCACATTTGACTGTTT |
| | GGCAAACTTTGACTACTGCTGCTAAATTCAGAACCCCAGAAAACAGAATTCCAGGT |
| | ATCTCAAGAGAAGATTACGCTAACGCTTTGACCGAAGTTTTTATGGCTACTTATGGT |
| | TTGTCCCATACCAAGAATACCAAGGTTGGTTCAGAATTGGTTAGAGGTGTTTCTGGT |
| | GGTGAAAGAAAGAGAGTTTCAATTGCCGAAGTTTCTTTAGCTGGTGCTAGATTGCA |
| | ATGTTGGGATAATGCTACTAGAGGTTTGGATGCTGCTACTGCTTTGGAATTCATTAG |
| | AGCTTTAAGAACCTCCGCTGATGTTTTGGATACAACTGCTTTGATTGCTATCTACCAA |
| | TGTTCCCAAGAAGCCTACGATTTGTTCGATAAGGTTTCTGTCTTGTACGAGGGTTAC |
| | CAAATTTTCTTTGGTAGAGCTGATAAGGCCAAAGAATACTTCATTAACATGGGTTGG |
| | GAATGCCCACCAAGACAAACTACAGCTGATTTCTTGACTTCTGTTACCTCTCCAAGA |
| | GAAAGAGTTCCAAGAGCTGGTTTTGAAAAGAAGGTTCCAAGAACTCCATCTGAATT |
| | TGCTACTTATTGGAAAGCTTCTCCAGAGTACAAGGCATTGATTGCCGAAATTGATGA |
| | ATCCTTGGCTGCCAATCAAAAGTCCGAATTGAAGGATTTGATCTACGATGCTAAGG |
| | CCTCCAGACAATCTAAAAGAATGAGAAAGACTGACCCCTACACCGTTTCTATTTCAT |
| | TGCAAACTAAGTACCTGTTGGAGAGAGAAGTCTACAGGATTAAGAACAACTTTGGT |
| | TTCCATGGCTTCTCCGCTATTGCTAATTCTTTGATGGCTTTGGTTTTGGCCTCCATCTT |
| | TTACAACATGTCTAAGACTACCGAGTCCTTCTATTCTAGAGGTGCTGCTATGTTTTTC |
| | GCTTGTTTGTTTAACGGTTTCCAGTCCTTCTTGGAGATCTTGTCTTTGTTTGAAGCCA |
| | GACCAATTATCGAAAAGCACAAACAATACGCCTTGTATCATCCAGCTGCTGAAGCTT |
| | TGGCTTCTGTTATTTCTCAATTGCCTTTTAAGGCCTTCTCCTCCTTGATGTTTAACCTG |
| | ATCTATTACTTCATGGTCAACTTCAGAAGAGATCCAGGTAGATTCTTCTTCTACTTGT |
| | TGGCTAACGTTACTTCTACCTTCACCATGTCTCATTTCTTCAGATTGATCGGCTCTAT |
| | GTCATCTACTTTGCCACAAGCTTTGGTTCCAGGTCATATAGTTTTGTTGGGTTTGTCC |
| | ATGTTCGTCGGTTTTACTATTCCAGTCAACTACATGTTAGGTTGGTGCAGATGGATT |
| | AACTACATTAACCCATTGGCTTACGCTTTCGAAGCTTTAATGGCTAACGAATTCCAT |
| | GGTTTGAGATACGCTTGTTCTGCATTTTTGCCAGATAACCCAGATAATCATCCAGAT |
| | TGGCCAGCTAAATCTTGGATCTGTAATGCTGTTGGTGCTGTTGCCGGTGAAGCTACT |
| | GTTTCAGGTGATGCTTACTTAGATGCTGCTTACTCCTACTCTAATTCTCATAAGTGGC |
| | GTAACTGGGCTATTACTTTTGCCTTCTGTATTTTCTTCTTGGCCACCTACATGATTTTC |
| | GCTGAGTATAATGAATCCGCCAAGCAAAAGGGTGAAATCTTGTTGTTTCAAAGGTC |
| | CACCTTGAAGAAGTTGAAGAAAGAACATAAGGCTGCCAAGAACGATATCGAAGGT |
| | GGTAAATTGAGAGACATCACCGAACAAGATCACGACGAAGAATCTGAACAACACG |
| | TTGATGCTATTCAAGCCGGTAAGGATATTTTCCATTGGAGAGATGTTCATTACACCG |
| | TCAAGATTAAGTCCGAGTACAGGGAAATTTTGTCTGGTGTTGATGGTTGGGTTAAG |
| | CCAGGTACTTTGACTGCCTTGATGGGTGCTTCAGGTGCTGGTAAAACTACTTTGTTG |
| | GATGTCTTGGCTAACAGAGTTACTATGGGTATCGTTACTGGTAACATGTTCGTTAAC |
| | GGTAGATTGAGGGATTCCTCATTCCAAAGATCTACTGGTTACGTTCAACAACAGGA |
| | CTTGCATTTGCCAACTGCTACTGTTAGAGAAGCCTTGAGATTTTCTGCTTACTTGAGA |
| | CAACCAGCTGAAGTCTCTAAAGCTGAAAAGGATGACTATGTCGAAGAGGTCATTAA |
| | GATCTTGGACATGCAAAAGTATGCTGATGCTGTTGTTGGAGTTGCTGGTGAGGGTT |
| | TGAATGTTGAACAAAGAAAAAGATTGACCATCGGTGTTGAATTGGCTGCTAAGCCT |
| | AAGTTGTTGTTGTTTTTCGATGAACCTACCTCCGGTTTGGATTCTCAAACTGCTTGGT |
| | CTATTTGCCAGTTGATGAGAAAGTTGGCTAATCACGGTCAAGCTATTTTGTGCACTA |
| | TTCATCAACCATCCGCCATCTTGATGCAAGAATTCGATAGATTGCTGTTCTTAGCCAA |
| | AGGTGGTAGAACTGTTTACTTTGGTGACTTAGGTGAAGGTTGCCAAACCTTGATTG |
| | ATTACTTTGAAAAGTACGGTGCTCCAAAGTGTCCTCCTGAAGCTAATCCAGCTGAAT |
| | GGATGTTGCATGTTATTGGTGCTGCTCCAGGTTCTCATGCTAATCAAGATTATCATC |
| | AAGTCTGGTTGGATTCCGCTGAAAGAAGAGATGTATTGTCTGAATTGGACAGGATG |
| | GAAAAAGAGTTGGTTAACATTCCAGTTGACGATTCCGTTTCTCACTCAGAATTTGCT |
| | GCACCATTTTGGGTTCAATTGACTGTTGTTACTGCCAGAGTGTTCCAACAATTTTGG |
| | AGAACACCATCTTACATTTGGGCCAAGATGTTTTTGTCCGTCGTTTCCTCTTTGTTTA |
| | TCGGCTTTATCTTCTTCAGGTCGAAGAACTCTATCCAAGGCTTGCAAAATCAAATGT |
| | TCGCCTTGTTCATGTTCCTGACCATTTTTAACCCACTGCTGCAACAAATTTTGCCCACT |
| | TTTGTTTCTCAGAGGGACTTGTACGAAACTAGAGAAAGACCAGCTAAGACCTTTTCA |
| | TGGAAGGCTTTCATTATCGCTCAATTCATAGCTGAAGCTCCATGGAATGCTTTTGTT |
| | GGTACAGTTGGTTTCTTCTGCTTTTATTACCCAGCTGGTTTCTACAGAAATGCCGAAC |
| | CATATGATGAAGTCAATGGTAGAGGTGCATATGGTTGGTTCTTTGCTGTTTTGTTCT |
| | TCATCTACATTGGTTCTATGGCCCATATGTTGATTGCCCCTTTACAAATTGCTGATTC |
| | TGCTGGTAATTTGGGCTCTTTGTTGTTCACTATGTGTTTGACTTTCTGCGGTGTTTTG |
| | GTTACTAAGGATGCTTTGCCAGGTTTTTGGGTTTTCATGTATAGAGTCTCTCCATTCA |
| | CCTACTTCATCGAAGGTTATTTGACTAATGCTTTGGCCCATAACAAGATCGTCTGTTC |
| | AGAAGAAGAGTTCAGAGTTTTGTCTCCACCAGATGGTTTGACTTGCCAAGATTATTT |
| | GGGTGACTACATTTCTAAAGCCGGTACAGGTTACTTGCAAGATCCAGAAGCAACTG |
| | GTTCTTGTCAATTTTGTCCAATGTCTAAAACGGACGACTTCTTGGCTCAAGTTCAATT |
| | GGATTATGGTAACAGATGGCGTGATGTCGGTATTTTCATTGCCTTCATTTTCATCAA |
| | CTTGTTCTTCGCCGTCCTGTTTTATTGGTTGGCTAGAGTTCCTAAGAAGTCCGATAG |
| | AGTTAGTACTGAACAACCTGAAGGTGCTGTTAATATGGGTGCTGAATTAGAAAAGA |
| | AAGCCGCCTTGCATAGAACTGCTACAAATGCTGCTTCACAAGCTGCTTCTCAAGGTT |
| | ATGCTCCACAAGTCTATAACGAAAAAGTCGGTTCTGAAGAAGGCTCCTTGGATAAG |
| | GTTGATAACTCTGATTCTTCCAGGTAA |
| SEQ ID | MSNSAYDIEDHPEEVNKYDGYNNAVDSEVQRLARQITQNSQLSFQDDGFKLAPGESNI |
| NO: 976 | DGLSRVSTIAPGVNPMQNVEELDPRLDPNSEEFQSRYWIKNFKALMDKDPDHYKNYSL |
| | GVTFKNLRASGEASDADYQTTIINAPFKIAKQYAKAVFSTRSAKQANRFDILKSLDGIVRP |
| | GEVLVVLGRPGSGCTTYLKSIASNTHGFKIGQESEMSYEGLTQKEIKKHFRGEVVYNAES |
| | DIHFPHLTVWQTLTTAAKFRTPENRIPGISREDYANALTEVFMATYGLSHTKNTKVGSEL |
| | VRGVSGGERKRVSIAEVSLAGARLQCWDNATRGLDAATALEFIRALRTSADVLDTTALI |
| | AIYQCSQEAYDLFDKVSVLYEGYQIFFGRADKAKEYFINMGWECPPRQTTADFLTSVTS |
| | PRERVPRAGFEKKVPRTPSEFATYWKASPEYKALIAEIDESLAANQKSELKDLIYDAKASR |
| | QSKRMRKTDPYTVSISLQTKYLLEREVYRIKNNFGFHGFSAIANSLMALVLASIFYNMSK |
| | TTESFYSRGAAMFFACLFNGFQSFLEILSLFEARPIIEKHKQYALYHPAAEALASVISQLPF |
| | KAFSSLMFNLIYYFMVNFRRDPGRFFFYLLANVTSTFTMSHFFRLIGSMSSTLPQALVPG |
| | HIVLLGLSMFVGFTIPVNYMLGWCRWINYINPLAYAFEALMANEFHGLRYACSAFLPD |
| | NPDNHPDWPAKSWICNAVGAVAGEATVSGDAYLDAAYSYSNSHKWRNWAITFAFCI |
| | FFLATYMIFAEYNESAKQKGEILLFQRSTLKKLKKEHKAAKNDIEGGKLRDITEQDHDEES |
| | EQHVDAIQAGKDIFHWRDVHYTVKIKSEYREILSGVDGWVKPGTLTALMGASGAGKTT |
| | LLDVLANRVTMGIVTGNMFVNGRLRDSSFQRSTGYVQQQDLHLPTATVREALRESAYL |
| | RQPAEVSKAEKDDYVEEVIKILDMQKYADAVVGVAGEGLNVEQRKRLTIGVELAAKPKL |
| | LLFFDEPTSGLDSQTAWSICQLMRKLANHGQAILCTIHQPSAILMQEFDRLLFLAKGGRT |
| | VYFGDLGEGCQTLIDYFEKYGAPKCPPEANPAEWMLHVIGAAPGSHANQDYHQVWL |
| | DSAERRDVLSELDRMEKELVNIPVDDSVSHSEFAAPFWVQLTVVTARVFQQFWRTPSY |
| | IWAKMFLSVVSSLFIGFIFFRSKNSIQGLQNQMFALFMFLTIFNPLLQQILPTFVSQRDLY |
| | ETRERPAKTFSWKAFIIAQFIAEAPWNAFVGTVGFFCFYYPAGFYRNAEPYDEVNGRGA |
| | YGWFFAVLFFIYIGSMAHMLIAPLQIADSAGNLGSLLFTMCLTFCGVLVTKDALPGFWV |
| | FMYRVSPFTYFIEGYLTNALAHNKIVCSEEEFRVLSPPDGLTCQDYLGDYISKAGTGYLQD |
| | PEATGSCQFCPMSKTDDFLAQVQLDYGNRWRDVGIFIAFIFINLFFAVLFYWLARVPKK |
| | SDRVSTEQPEGAVNMGAELEKKAALHRTATNAASQAASQGYAPQVYNEKVGSEEGSL |
| | DKVDNSDSSR |
| SEQ ID | ATGTCCTTGAACAACACCCCAAACCATCACCAAGATGATTCTGATCATTCA |
| NO: 977 | GTTCCAGATGTTCCAGAAGAGTACAAGGGTTTCGATCAAAACGTTGATGT |
| | TCACATTCAAAACTTGGCCAGACAAATTACCAATGCTTCTCAAGCTAACAC |
| | CTCCTTGTCTAGAGTTTCTTCTATTGCTCCAGGTGTTGTTGCTATTAACAA |
| | CCCAGATGTTGATCCAAGATTGGACCCAAATTCTGACAACTTCGAATCTA |
| | GATACTGGATCAAGAACTTCAAGAACCTGATGGATAAGGATCCAGATCAT |
| | TACGCTAACTACTCATTGGGTATCGTCTACAAGAATTTGAGAGCTTTTGGT |
| | GAAGCTACCGATGCTGATTATCAAACTACTGTTTTGAACATGCCATTGAAG |
| | TACGCTGGTAAAGCTTTGAAATACGCCTTGTCATCTAGATCTGCTAAGAA |
| | GGCTAAGCAATTCGATATCTTGAAACCTATGGACGCCTTGATTAAGCCAG |
| | GTGAAGTTGTTGTTGTTTTGGGTAGACCAGGTTCTGGTTGTTCTACTTTGT |
| | TGAAAACCATTGCCTCTAACACCCATGGTTTCCATATTGGTGAAGAATCC |
| | GAAATCTCTTACGAAGGTTTGACCCAAAAGGATATCAAGAGACATTATAG |
| | GGGTGAAGTTATCTACAACGCCGAAACCGATATTCATTTCCCACATTTGA |
| | CTGTTTGGCAGACTTTGTCTTTGGCTGCTAAATTCAGAACTCCACAAAACA |
| | GAATTCCAGGCATCTCAAGAGAAGATTACGCCAATCATTTGACCGAAGTT |
| | TACATGGCTACTTACGGTTTGTCTCATACCAAGAATACCAAGGTTGGTAAC |
| | GAAAACGTTAGAGGTGTTTCTGGTGGTGAAAGAAAGAGAGTTTCAATTGC |
| | CGAAGTTTCTTTGTCTGGTGCTAGATTGCAATGTTGGGATAATGCTACTAG |
| | AGGTTTGGATGCTGCTACTGCTTTGGAATTCATTAGAGCTTTGAGAACCC |
| | AAGCCGATATTTTGGATACAACTGCTTTCGTTGCTATCTACCAGTGTTCTC |
| | AAGATGCTTACGATTTGTTCGATAAGGTGTCTGTCTTGTATGAAGGTTACC |
| | AAATCTACTTCGGTAGAGCTGATGAAGCTAAGGATTACTTTGTTAGAATGG |
| | GTTACCATTGTCCAGCTAGACAAACTACAGCTGATTACTTGACTTCTATCA |
| | CCTCTCCAAGAGAAAGAGTTGCTTCTAAGGGTTTTGAAAACAAGGTTCCA |
| | AAGACGCCAAAAGAGTTTGAAACTTACTGGAAAGCTTCTCCTGAATACGC |
| | TAAGTTGGTTCAAGAAATTGATGCCACCTTGCAAAACCACGATGATAATTC |
| | TAAGGCCATTATCAAGTCCGCTCACAATCAAAAGCAAGCTAAACATATGA |
| | GACACACCTCACCATACACTGTTTCATTTTGGATGCAAGTCAGATACTTGT |
| | TGACCAGAGATTTCCAGAGAATCAGAAACGATTTGGGCTTCAACTTGTTT |
| | CAAGTTTGGGCTAATTCTCTGATGGCCTTGATTTTGTCCTCCATCTTTTAC |
| | AACATGCAGTCTAACACTGGCAGCTTTTATTACAGAGGTGCTGCTATGTTT |
| | TTCGCCGTTTTGTTTAATGGCTTCTCGTCCTTCTTGGAAATCATGACTTTG |
| | TTTGAAGCCAGGCCAATTATCGAAAAGCACAAGCAATACTCGTTGTACCA |
| | TCCATCTGCTAACGCTTTATCTTCCGTTTTTAGTCAAGTCCCTTCTAAGAT |
| | GGTTACCTCTGTTGCTTTTAACTTGGTCTTTTACTTCATGGTCAACTTCAG |
| | AAGAAACCCAGGTAGATTCTTCTTCTACTACTTGATGAACTTGACCGCTAC |
| | CTTTTCCATGTCTCATTTCTTTAGATTGGTTGGTTCTGCTGCTTCTTCATTG |
| | CCAGAAGCTTTGGTTCCAGCTCATATTATCTTGTTGGCTTTGACTATTTTC |
| | ACCGGTTTCGTTATCCCAGTCAACTATATGTTAGGTTGGTCCAGATGGAT |
| | CAACTACTTGGATCCATTGGCTTATGCTTTCGAAGCTTTGATGGCAAATGA |
| | ATTCGCTGGTAGAGAATTCGAATGCTCCCAATTCATTCCAGGTGATCCTA |
| | GAACTACTCCAGGTATTCCAGATGATGGTTTCATTTGCTCTGTTGTCTCTT |
| | CTGTTCCAGGTTCTTTTGTTGTTGATGGTTCTAGGTACTTGGAGGTCAATT |
| | ACAAGTACAAGAACTCTCATAAGTGGCGTAACTGGGGTATTACTTTGGCT |
| | TTCACTCTGTTTTTCTTGTTCGTCTACTTGGTGTTCTCTGAGTACAATGAAT |
| | CTGCTATGCAAAAGGGTGAAGTCCTGTTGTTTCAAAGATCTACCTTGCGT |
| | AAGTTGAAAAAGCAGCATGGTGATATTAAGAACGACTTGGAAGCTGGTTC |
| | TGAAAGAGATGTTACTGAACAAGATGAAGAAGAAGGCGAACAAAACGTAG |
| | ACGTTATTCATGCTGGTACTGATATCTTCCATTGGAGAGATGTTCACTACT |
| | CCGTCAAGATTAAGAAAGAAACCAGGGAAATCTTGAACGGTGTTGACGGT |
| | TGGGTTAAGCCTGGTACTTTGACTGCTTTAATGGGTGCTTCTGGTGCTGG |
| | TAAAACTACCTTGTTGGATGTTTTGGCTAACAGAGTTACTATGGGTGTTGT |
| | CACTGGTAACATGTTTGTTAACGGTCACTTGAGAGACAACTCATTCCAAA |
| | GATCAACTGGTTACGTCCAACAACAAGACTTGCATTTGAAAACTGCTACT |
| | GTTAGAGAAGCCTTGCAATTCTCTGCTGATTTGAGACAACCTAAAGAAGT |
| | CTCTAAGGCTGAAAAAGATGCCTACGTTGAAGAAGTCATTAAGATCCTGG |
| | ATATGGAAAAGTACGCAGATGCTGTTGTTGGTGTTGCTGGTGAAGGTTTA |
| | AATGTCGAGCAAAGAAAAAGATTGACCATCGGTGTTGAATTAGCTGCTAA |
| | ACCTAAGCTGTTGCTGTTTTTGGATGAACCTACTTCTGGTTTGGATTCTCA |
| | AACTGCTTGGTCTATTTGCCAGTTGATGAGAAAGTTGGCTAACCATGGTC |
| | AAGCTATTTTGTGCACTATTCATCAACCATCCGCTATCTTGATGCAAGAAT |
| | TCGATAGATTGCTGTTCTTGGCTAAAGGTGGTAGAACTGTTTACTTTGGTG |
| | ATTTGGGTGAAAACTGTCAGTCCTTGATTGACTACTTCGAAAAGTATGGTG |
| | CTCCAAAATGTCCACCACAAGCTAATCCAGCTGAATGGATGTTGCATGTT |
| | ATTGGTGCTGCACCAGGTTCACATGCTAATCAAGATTATCACCAAGTCTG |
| | GTTGGAATCCTCTGAAAGACAAGATGTATTGAACGAATTGGACAGGTTGG |
| | AAACCGAATTGGTTAAGTTGCCAAGAGATGACTCTATCGGTCAAGAAGAA |
| | TTTGCTGCTCCATTGTGGAAACAATACTTGATTGTCACCAAGAGAATCTTG |
| | CAACAGCATTGGAGATCACCAGTTTACATTTGGTCTAAGTTGTTCCTAGCC |
| | GTGTCCTCTTCTTTGTTTATTGGTTTTGCTTTCTTCAAGGCTAAGAACACT |
| | CAACAGGGATTGCAAAATCAAATGTTCGGCATCTTCATGTACCTGATCATT |
| | TTCAACCCATTGGTCCAACAAACTTTGCCAGCTTTTGTTGAACAAAGAGCC |
| | TTGTACGAAACTAGAGAAAGACCATCTAAGACCTTTTCATGGAAGGCTTTT |
| | ATTGCTGCCCAAATTACCTCTGAAATTCCTTGGAATGCTTTGGTTGGTACT |
| | ATTGCCTTTCTGTGTTTTTACTACCCAGTCGGTTTCTACAACAATGCCTCT |
| | CCAACTAATGCAGTTGATAAGAGAGGTGCTTACGCTTGGTTTTTCAATGTT |
| | TTGTTCTACGTTTACATCGGCACCATGGCTCATTTGTGTATTGCTGGTTTA |
| | GAATTGGCTGATGCTGCTGGTAATATTGCTTCATTGGCTTTTACCTTGTGT |
| | TTGACCTTTTGCGGTGTTTTAGTTGGTCCAAAAGCCTTACCAGGTTTCTGG |
| | ATTTTTATGTACAGGGCTAATCCATTCACCTACTTCATAGATGGTTTCTTGT |
| | CTAATGCCCTGGCTAACAATAGAGTTCAATGTTCCACTCAAGAATACGTC |
| | CATTTTAATCCACCATCTGGTTATACTTGTGGTCAGTACATGCAATCCTAC |
| | ATCGAAAAAGCTGGTACAGGTTATTTGTCTGATCCAGATGCTACTACTGAT |
| | TGCAATTTCTGTGCTATGTCTACTACCAACGCCTTCTTGAAATTCGTTTCC |
| | TTGGATTATTCTAGGCGTTGGAGAAACGTTGGTATTTTCATTGCCTTCATT |
| | TTCATCAACATTATCGGCGCCACATTCTTTTTCTGGTTGGCTCGTGTTCCT |
| | AAAAAGGCTGATAGAGTTAAGGGTCAAGCCAAAGAAAGATGTGCTGAAAG |
| | AGCTGCTCAAAAACAAGAGACTAACTCCTTCAACGAGAAAGAGGAATCTT |
| | CTACCAACTAA |
| SEQ ID | MSLNNTPNHHQDDSDHSVPDVPEEYKGFDQNVDVHIQNLARQITNASQANTSLSRVS |
| NO: 978 | SIAPGVVAINNPDVDPRLDPNSDNFESRYWIKNFKNLMDKDPDHYANYSLGIVYKNLR |
| | AFGEATDADYQTTVLNMPLKYAGKALKYALSSRSAKKAKQFDILKPMDALIKPGEVVVV |
| | LGRPGSGCSTLLKTIASNTHGFHIGEESEISYEGLTQKDIKRHYRGEVIYNAETDIHFPHLT |
| | VWQTLSLAAKFRTPQNRIPGISREDYANHLTEVYMATYGLSHTKNTKVGNENVRGVSG |
| | GERKRVSIAEVSLSGARLQCWDNATRGLDAATALEFIRALRTQADILDTTAFVAIYQCSQ |
| | DAYDLFDKVSVLYEGYQIYFGRADEAKDYFVRMGYHCPARQTTADYLTSITSPRERVAS |
| | KGFENKVPKTPKEFETYWKASPEYAKLVQEIDATLQNHDDNSKAIIKSAHNQKQAKHM |
| | RHTSPYTVSFWMQVRYLLTRDFQRIRNDLGFNLFQVWANSLMALILSSIFYNMQSNTG |
| | SFYYRGAAMFFAVLFNGFSSFLEIMTLFEARPIIEKHKQYSLYHPSANALSSVFSQVPSKM |
| | VTSVAFNLVFYFMVNFRRNPGRFFFYYLMNLTATFSMSHFFRLVGSAASSLPEALVPAH |
| | IILLALTIFTGFVIPVNYMLGWSRWINYLDPLAYAFEALMANEFAGREFECSQFIPGDPRT |
| | TPGIPDDGFICSVVSSVPGSFVVDGSRYLEVNYKYKNSHKWRNWGITLAFTLFFLFVYLV |
| | FSEYNESAMQKGEVLLFQRSTLRKLKKQHGDIKNDLEAGSERDVTEQDEEEGEQNVDV |
| | IHAGTDIFHWRDVHYSVKIKKETREILNGVDGWVKPGTLTALMGASGAGKTTLLDVLA |
| | NRVTMGVVTGNMFVNGHLRDNSFQRSTGYVQQQDLHLKTATVREALQFSADLRQPK |
| | EVSKAEKDAYVEEVIKILDMEKYADAVVGVAGEGLNVEQRKRLTIGVELAAKPKLLLFLD |
| | EPTSGLDSQTAWSICQLMRKLANHGQAILCTIHQPSAILMQEFDRLLFLAKGGRTVYFG |
| | DLGENCQSLIDYFEKYGAPKCPPQANPAEWMLHVIGAAPGSHANQDYHQVWLESSER |
| | QDVLNELDRLETELVKLPRDDSIGQEEFAAPLWKQYLIVTKRILQQHWRSPVYIWSKLFL |
| | AVSSSLFIGFAFFKAKNTQQGLQNQMFGIFMYLIIFNPLVQQTLPAFVEQRALYETRERP |
| | SKTFSWKAFIAAQITSEIPWNALVGTIAFLCFYYPVGFYNNASPTNAVDKRGAYAWFEN |
| | VLFYVYIGTMAHLCIAGLELADAAGNIASLAFTLCLTFCGVLVGPKALPGFWIFMYRANP |
| | FTYFIDGFLSNALANNRVQCSTQEYVHFNPPSGYTCGQYMQSYIEKAGTGYLSDPDATT |
| | DCNFCAMSTTNAFLKFVSLDYSRRWRNVGIFIAFIFINIIGATFFFWLARVPKKADRVKG |
| | QAKERCAERAAQKQETNSFNEKEESSTN- |
| SEQ ID | ATGAAGGCCAACAAGTCTAACATGGCTGGTGATCCACATGGTTCTTGTGATTCTGCT |
| NO: 979 | GTTTTGACTAGAAACACCTCTCCAGAATTGCCAGAATATGATGGTTTGGATTGCTCT |
| | GCTAGAGCTTCTGTTAGAGAATTGGCTAGAACTTTCACTAACGGTTCTGTTGCTTCT |
| | AATTCTAAAGCTGCTGCTGTTGGTGTTCATGCTACTACTGCTTTGCCAGCTTCTAGAA |
| | CTACTACTGAATATGGTGATGTCAACACTACGAACCCAGTTTTCTCTTCTTCTGAATT |
| | GCCATCCTACAACTCTAGATTGGACCCAAATTCTGACGAATTCTCTTCTGCTTTGTGG |
| | GTCAAGAATTTGTCCCAATTGATTGCTTCTGATCCAGATCACTACAAGCCATATTCTT |
| | TGGGTTGTACTTGGAAGAACTTGAGAGCTTATGGTAACGCTACTGATGTTGCTTACC |
| | AATCTACTTTTGCTAACTTGCCATTGCAGTTGTTGGAATCTGGTTATAGAGCTGCTA |
| | GAAAAGCTAGACCAGAAGATTCCTTCGATATCTTGAAACCTATGGATGGTATCGTTA |
| | AGCCATCAGAGTTGTTGGTTGTTTTGGGTAGACCAGGTTCTGGTTGTTCTACTTTGT |
| | TGAAGTCTATTTCCGCTAACACCCATGGTTTCCATATCGATTCTGATTCCGAAATCTT |
| | CTACGATGGTATGGACCCTAAAGAAATTGCTAAACACTACAGAGGTGAAGTTGTTT |
| | ACAACGCCGAATCTGATGTTCATTTCCCACATTTGACTGTTTTCGACACCTTGAAAAC |
| | TGTTGCTAGATTGTCTTGTCCATCCAACAGATTCCATGGTGTTGATAGAGAAACTTT |
| | CGCTACCCATATTACCGAAGTTGCTATGGCTACTTATGGTTTGTCTCATACCAGAAA |
| | CACTAAGGTCGGTTCAGAATTGGTTAGAGGTGTTAGTGGTGGTGAAAGAAAGAGA |
| | GTTTCTATTGCCGAAGTCTCTATTTGCGGTTCTAAGTTTCAATGTTGGGATAATGCTA |
| | CCAGAGGTTTAGATTCTGCTACAGCCTTGGAATTCATTAGAGCTTTGAGAACTACTG |
| | CCAAGTTGAACAATTCTGCTGGTGTTATTGCTATCTACCAATGCTCTCAAGATGCCTA |
| | CGATTTGTTCGATAAGGTTTGTGTTTTACACGAGGGTTATCAGATCTATTTTGGTCC |
| | AGCTAATGAAGCCAAGCAATACTTTTTGGATATGGGCTATGTTTCCCCAGATAGACA |
| | AACTACTGCTGATTTCTTGACTGCTGTTACTAATCCAGCTGAAAGGATCGTCAATCA |
| | AGAAATGGTTCAAGCTGGTAAGGTTGTTCCATCTTCTGCATCTGAAATGGAAGCTCA |
| | TTGGAAGCAATCTGAAAACTACAAGAGACTGATCAACGAAATCGACCATTACACTA |
| | CTCATGATCAAACCGGTAACAGAGAACAATTGAGAAACGCTCATATCGCTAAGCAA |
| | TCTAAGAGAGCTAGACACTCTTCTTCATACACTGTCTCTTACGGTTTACAGGTGAAG |
| | TACTTGTTGATCAGAAACATGCAGAGGATCAGATCTTCTATGGGTGTCACTTTGTTC |
| | CAAGTTATTGGTAATGGTGGTATGGCTTTCATCTTGGGTTCTATGTTCTACAAGATTT |
| | TGAAGCACGATACCACTGCTGGTTTTTATTCTAGAGCTGGTGCTTTGTTTTTCGCCGT |
| | TTTGTTTAATGCTTTCTCGTGCTTGTTGGAAATCTTGGCATTATATGAAGCCAGGCCA |
| | ATTTCTGAAAAGCACAAGCGTTATTCCTTGTACCATCCTTCTGCTGATGCTTTGGCTT |
| | CAGTTATTAGTGAAATCCCTTCTAAGTTGGTTACCTCCGTTGTTTTTAACTTGGCCTT |
| | GTACTTCTTGTGCAACTTTAAACGTGAAGCTGGTGCCTTTTTCTTCTACTTCTTGATG |
| | ACTATCGTTGCCACGTTTTTGATGTCCCATATCTTTAGATGTTTGGGTGCTGCTACTA |
| | AGACTTACGCTGAATCTATGGTTTCTGCCTCTTTGTTGTTGTTGGCTCAAGCTATCTA |
| | TACCGGTTTTGCTATTCCAAAGACCAACATCTTAGGTTGGTCTAAGTGGATTTGGTA |
| | CATCAACCCATTGTCCTACATCTTCGAATCCTTGATGGTCAACGAATTTCACGGTAG |
| | AAACTTTTCTTGCTCCCAGTATATTCCAGCTGGTTCAGGTTACGAAATTTTGTCTGGT |
| | ACTGAAAGAGTTTGTTCTGCAGTTGGTGCTGTTCCAGGTCAAGATTTCGTTTCTGGT |
| | GAAACCTACATTAACGTTGCTTACGGTTATTACCATGCTCATAAGTGGCGTGGTTTA |
| | GGTATTGGTTTGGCTTATGCTATTGTGTTCTTGGCTGTTTATTTGGCCGTTACCGAAT |
| | TCAATGAATCTGCTAAGCAAAGGGGTGAGATTTTGGTTTTTCCACAATCCGTTATGC |
| | GTAGGATGAAGAAAGAACGTAAACTGAGAAACTCCTCCGATTACTCTGGTACAGAT |
| | GTTGAAAATTCAGCAGGTTCTGCTCCATTGAACGAGAAGAAAATGTTGGATGAATC |
| | CTCTGTTTCTGCCGGTTCTACTTCATCAATGGGTGATGCTAAATTGTCCAAGTCTGAA |
| | GCCATATACCATTGGAGAAACGTTTGCTTCGAAGTCAACATCAAGAAAGAAACCAG |
| | AAGGATCTTGAACAACGTTGATGGTTGGGTTAAGCCAGGTACTTTGACTGCTTTAAT |
| | GGGTGCTTCTGGTGCTGGTAAAACTACTTTATTGGACTGTTTGGCTTCTAGAGTTAC |
| | CACTGGTACTATTACTGGTGACATGTTCATTAACGGTTTCTTGAGAGATGCTTCCTTC |
| | GCTAGATCTATTGGTTACTGTCAACAACAAGACTTGCATTTGGAATCCGCTACTGTA |
| | AGAGAATCTTTGAGATTTGCTGCCTACTTGAGACAACCAGCTACTGTTTCTGAAGAT |
| | GAGAAGAACAAGTACGTGGAAGATGTCATTAAGATCTTGGAGATGGAAACTTACG |
| | CTAACGCTGTTGTAGGTGTTGCTGGTGAAGGTTTGAATGTTGAACAGAGAAAAAGA |
| | TTGACCATCGGTGTTGAATTGGCTGCTAAGCCTAAGTTGTTGTTATTCTTGGATGAA |
| | CCTACCTCTGGTTTGGACTCTCAAACTGCTTGGTCTATTTGTCAGTTGATGAGAAAG |
| | TTGGCTAACCATGGTCAAGCTATTTTGTGCACTATTCATCAACCATCCGCTTTGTTGA |
| | TGCAAGAATTCGATAGACTGTTGTTCTTGCAAAGAGGTGGTCAAACTGTTTACTTCG |
| | GTGAATTAGGTAAGGGTTGCCATAAGATGATCGACTACTTTGAATCTAATGGTGCT |
| | CCAAGATGTCCAGATGGTGCTAATCCTGCAGAATGGATGTTGGCTGTTATTGGTGC |
| | TGCTCCTGGTACTCATGCTAATCAAGATTATCATGAAGTCTGGCGTAATTCCCCAGA |
| | GTACAGAGCTGTTCAAGAGGAATTGGAATGGATGGAACAAGAATTGCCTAAGAAG |
| | CCAATTGACACCTCCAATGAACAAACTGAATTTGCCGCTTCTCTGTTGTATCAGTACT |
| | ACTTGGTTACTAAGAGATTGGCCGAACAATATTGGAGAACTCCATCTTACTTGTGGT |
| | CCAAGTTGATCTTGTCCGTTATCTCCCAAATTTTCATCGGCTTCACTTTCTTCAAGGC |
| | CGATTCTTCATTGCAAGGTCTGCAAAATCAAATGCTGTCCATTTTCATGTTCACCCTG |
| | GTTTTCAACCCTACCTTGCAACAATATTTGCCAACCTTTGTTTCTCAGAGAGGTCTAT |
| | ACGAAGCTAGAGAAAGACCATCTAAGACCTTTTCTTGGGTGTCTTTCATGTTGTCCC |
| | AAATCACTGTTGAAATCCCATGGAATATTTTGGCCGGTACTATTGGTTTCATCATCTA |
| | CTATTATCCAGTCGGCTTTTACAACAACGCTTCAAAAGCTGGTCAATTGCACGAAAG |
| | AGGTGCTTTATTTTGGTTGTACTGTACCGCTTTCTACGTTTTCACTGGTTCTATGGCT |
| | CAAGTATGTATTGCAGGTTTGGATGTAGCTGAAGCAGCTGGTGAATTGGGTAGTTT |
| | GTTGTATACTTTGGCTTTGTCTTTCTGCGGTGTCATGGTTACTCCATCTAATATGCCT |
| | AGATTCTGGCTGTTCATGTACAGAGTTTCACCAATCACCTACTTCATTGACGGTGTTT |
| | TGTCAACTGGTGTTGCTAATGCTGATGTACATTGTGCTGATTACGAAATGGTTAGAT |
| | TCACTCCACCAGCAGGTCAAACATGTGGTCAGTATATGTCTAGATATATCGAAACTA |
| | CCGGTACTGGTTACTTGGATGATCCATCTGCTATGGATGAATGCAAGTTCTGTTCTG |
| | TCTCTGATACCAATGAATTCTTGAAGGCCGTTACATCCTCTTATGATCATCGTTGGAG |
| | AAATTACGGTATCTTCCTGGTCTTTATCTTCGTCAATTTTGCTTTGGCCTCTTTCTTGT |
| | ACTGGCTAATGAGAGTTCCAAAGAAGAGGAATAGAGTTGTCGACGAAAGAAAACC |
| | TGAAGCTCAAAAATTGGCCAGCAAGTGA |
| SEQ ID | MKANKSNMAGDPHGSCDSAVLTRNTSPELPEYDGLDCSARASVRELARTFTNGSVAS |
| NO: 970 | NSKAAAVGVHATTALPASRTTTEYGDVNTTNPVFSSSELPSYNSRLDPNSDEFSSALWV |
| | KNLSQLIASDPDHYKPYSLGCTWKNLRAYGNATDVAYQSTFANLPLQLLESGYRAARKA |
| | RPEDSFDILKPMDGIVKPSELLVVLGRPGSGCSTLLKSISANTHGFHIDSDSEIFYDGMDP |
| | KEIAKHYRGEVVYNAESDVHFPHLTVFDTLKTVARLSCPSNRFHGVDRETFATHITEVA |
| | MATYGLSHTRNTKVGSELVRGVSGGERKRVSIAEVSICGSKFQCWDNATRGLDSATAL |
| | EFIRALRTTAKLNNSAGVIAIYQCSQDAYDLFDKVCVLHEGYQIYFGPANEAKQYFLDM |
| | GYVSPDRQTTADFLTAVTNPAERIVNQEMVQAGKVVPSSASEMEAHWKQSENYKRLI |
| | NEIDHYTTHDQTGNREQLRNAHIAKQSKRARHSSSYTVSYGLQVKYLLIRNMQRIRSSM |
| | GVTLFQVIGNGGMAFILGSMFYKILKHDTTAGFYSRAGALFFAVLFNAFSCLLEILALYEA |
| | RPISEKHKRYSLYHPSADALASVISEIPSKLVTSVVFNLALYFLCNFKREAGAFFFYFLMTIV |
| | ATFLMSHIFRCLGAATKTYAESMVSASLLLLAQAIYTGFAIPKTNILGWSKWIWYINPLSY |
| | IFESLMVNEFHGRNFSCSQYIPAGSGYEILSGTERVCSAVGAVPGQDFVSGETYINVAYG |
| | YYHAHKWRGLGIGLAYAIVFLAVYLAVTEFNESAKQRGEILVFPQSVMRRMKKERKLRN |
| | SSDYSGTDVENSAGSAPLNEKKMLDESSVSAGSTSSMGDAKLSKSEAIYHWRNVCFEV |
| | NIKKETRRILNNVDGWVKPGTLTALMGASGAGKTTLLDCLASRVTTGTITGDMFINGFL |
| | RDASFARSIGYCQQQDLHLESATVRESLRFAAYLRQPATVSEDEKNKYVEDVIKILEMET |
| | YANAVVGVAGEGLNVEQRKRLTIGVELAAKPKLLLFLDEPTSGLDSQTAWSICQLMRKL |
| | ANHGQAILCTIHQPSALLMQEFDRLLFLQRGGQTVYFGELGKGCHKMIDYFESNGAPR |
| | CPDGANPAEWMLAVIGAAPGTHANQDYHEVWRNSPEYRAVQEELEWMEQELPKKP |
| | IDTSNEQTEFAASLLYQYYLVTKRLAEQYWRTPSYLWSKLILSVISQIFIGFTFFKADSSLQ |
| | GLQNQMLSIFMFTLVFNPTLQQYLPTFVSQRGLYEARERPSKTFSWVSFMLSQITVEIP |
| | WNILAGTIGFIIYYYPVGFYNNASKAGQLHERGALFWLYCTAFYVFTGSMAQVCIAGLD |
| | VAEAAGELGSLLYTLALSFCGVMVTPSNMPRFWLFMYRVSPITYFIDGVLSTGVANADV |
| | HCADYEMVRFTPPAGQTCGQYMSRYIETTGTGYLDDPSAMDECKFCSVSDTNEFLKA |
| | VTSSYDHRWRNYGIFLVFIFVNFALASFLYWLMRVPKKRNRVVDERKPEAQKLASK |
| SEQ ID | ATGGACAAGGACAACGAAAACGAAATCGAGGAACCTAATGGTTACGAAGT |
| NO: 981 | TTCTTCTGCTTCTGGTAGATATGAAGGTTTGGATCAAACTGCTGCCCAATC |
| | CATTAAGGATTTGGCTCAATCTTTGTTGGAAGAGTCCAGAGAATCTATTCA |
| | TGCTGCTGGTGAAGGTGTTAATCCAGTTTTTATGTCTGAATCCGGTGATG |
| | ACTACAACCCAAAAGTTGATCCAGCTAACGATGAGTTCTCTAGCAAAGAA |
| | TGGATCAAGAACTTGTCCACCATCATCAAATCTGACCCATCTTTTTACAAG |
| | CCATACACCTTGGGTTGTTCTTGGAGAGATTTGTCTGCTGTTGGTGCTTC |
| | TGCTGATGTTGCTTATCAAACTACTTTCGAAAACTTGCCATGGAAGGCTTT |
| | GACTTGGGTTTACAGAAGATTGAAGGCTGCTAACGAATCTGATACCTTCC |
| | AAATTTTGAAGCCAATGGATGGTATTGTCAATCCAGGTGAATTATTGGTTG |
| | TTTTGGGTAGACCAGGTTCTGGTTGTACTACTTTGTTGAAGTCCATTTCCT |
| | CTAACACCCATGGTTTCAAGGTTACCGAAGATTCCAAGATTTCTTACTCTG |
| | GTTTGTCCCCAAAGGATATCAAGAGACATTTTAGAGGTGAAGTTGTCTAC |
| | AACGCCGAATCCGATATTCATTTGCCACATTTGACTGTCTACCAAACCTTG |
| | TTGACTGTTGCTAGATTGAAAACCCCACAAAACAGAATTCCAGGTGTCGA |
| | TAGAGAATCTTGGGCTAAACATGTTACTGAAGTTGCTATGGCTACTTACG |
| | GTTTGTCTCATACAAGAAACACAAAGGTTGGTGGTGATTTGGTTAGAGGT |
| | GTTTCTGGTGGTGAAAGAAAGAGAGTTTCTATTGCCGAAGTTACCATTTG |
| | CGGTTCTAAGTTTCAATGTTGGGATAACGCTACCAGAGGTTTAGATTCTG |
| | CTACTGCTTTGGAATTCATTAAGGCCTTGAAAGCTCAAGCCGAAATAGTT |
| | GATTCTGCTGCTTGTGTTGCTATCTACCAATGTTCTCAAGATGCCTACGAT |
| | TTGTTCGATAAGGTTTGTGTCTTGTACTCCGGTTACCAAATCTATTTTGGT |
| | TCTGCTAAGAACGCCAAGAGATACTTTCAACAAATGGGTTACTACTGCCC |
| | ATCTAGACAAACTACTGCTGATTTCTTGACCTCTATTACTTCTCCAGCCGA |
| | AAGAATTATCAACAACGGTTACATTGAGAAGGGTATCAACGTTCCACAAA |
| | CTCCAGAAGAAATGTCTGATTACTGGAAGAACTCTCCAGAATACGAGAAT |
| | TTGGTCAGAGAAACCGACGAATTCATCTCCCAAGATCATAACTCTAAGAT |
| | CAGCTCCATTAGAGAAGCACATCAAGCTAGACAATCTAAGAGAGCTAGAC |
| | CAGCTGAACCATATACTGTTTCTTATTTGATGCAGGTCAAGTACCTGCTGA |
| | TCAGAAACATTTGGAGAATCCAGAACTCCTACTCTATTACCGCTTTCCAAG |
| | TTATCGGTCATTCTGTTATGGCTTTGTTGTTGGGTTCTATGTTCTACAAGG |
| | TTATGAAGCACACTTCTACCGATACCTTTTACTATAGAGGTTCCGCTATGT |
| | TCTTCGCCGTTTTGTTTAATGCTTTCTCCTCCTTGTTGGAAATCTTCAGCTT |
| | GTATGAAGCCAGACCAATTACTGAAAAGCACAGAACCTACTCGTTGTATA |
| | GACCTTCAGCTGATGCTTTCGCTTCCATTTTGTCTGAAATTCCAGCTAAGA |
| | TTTTGACCGCCATCTTCTTTAATTTGGCCTACTACTTCTTGGTCAACTTCA |
| | GACGTGATGGTGGTAGATTTTTCTTCTACTTCCTGATCAACATCGTCGGTA |
| | CTTTCACTATGTCTCATTTGTTCAGATGCGTTGGTTCTTTGACTAAGACTTT |
| | GACTGAAGCTATGATTCCAGCCTCCATCTTGTTGTTAGGTATGGCTATGTA |
| | TGCTGGTTTCGCTATTCCAGAAACTAAGATGTTAGGTTGGTCTAAGTGGA |
| | TCTGGTACATTAACCCATTGTCCTACTTGTTCGAAGCTTTGATGACTAACG |
| | AATTCCACGATAGAAAGTTCCCATGTGCTACTTTTATTCCATCAGGTGGTG |
| | AATACGAACAGTTCAGAGGTAAAGAAAGAATCTGTGGTGTTGTTGGTTCA |
| | GTCCCAGGTGAAAATTATGTATTGGGTGACAACTTCCTGAAAAAGTCCTA |
| | CAACTACGATATCGAACATAAGTGGCGTGCTTTTGGTGTTGGTATGGCAT |
| | ACGTTATTTTCTTTTTCTTCGTGTACCTGTTCCTGTGCGAAATCAATGAAG |
| | GTGCTAAACAAAAGGGTGAGATCTTGGTTTTTCCACAGACTGTTGTTAGA |
| | AAGATGAGGAAGCAAAAGAAGATCAGATCCAGAACAAACGCCTTGAACGA |
| | TTTGGAAAAGAACATCGGTACTAATGCTACCAACTTGACTGATACCACTCT |
| | GGTCAAAGAATCTTCCGATTCTACTGATGAAGTCCAAGAGCAATCTGGTT |
| | TGACAAAGTCTAAGGCTATTTTCCATTGGAGGAACTTGTGCTACGATGTTC |
| | AAATCAAAGCTGAAACCAGAAGGATCTTGTCTGACGTTGATGGTTGGGTT |
| | AAGCCAGGTACTTTAACTGCTTTGATGGGTTCTTCTGGTGCTGGTAAAAC |
| | TACATTATTGGATTGCTTGGCTGAAAGGGTTACTATGGGTGTAATTACCG |
| | GTGATATTTTCGTCAACGGTAAGAGAAGAGATGCCTCATTTCCAAGATCTA |
| | TTGGTTACTGTCAACAACAGGACTTGCATTTGAAAACTGCTACTGTTAGAG |
| | AGTCCTTGTTGTTCTCTGCTATGTTGAGACAACCTAAGAACGTTCCAACTA |
| | GCGAAAAGAAGAAGTACGTTGAAGAAGTCATCAAGATCCTAGAGATGGAA |
| | CCATATGCTGATGCAGTTGTTGGTGTTGCCGGTGAAGGATTGAATGTTGA |
| | ACAGAGAAAAAGATTGACCATCGGTGTTGAATTGGTTGCTAAACCTAAGC |
| | TGTTGGTTTTCTTGGATGAACCTACAAGTGGTTTGGATTCTCAAACAGCTT |
| | GGTCTATTTGCCAGTTGATGAGAAAGTTGGTTAACAGAGGACAAGCTATT |
| | TTGTGCACTATCCATCAACCATCCGCAATCTTGATGCAAGAATTTGATAGG |
| | TTGCTGTTCTTGCAAAAAGGTGGTGAGACTGTTTACTTTGGTGAATTGGG |
| | TGATGAATGCTCCATCATGGTTGATTACTTTGAAAGAAACGGTGCTCATAA |
| | GTGTCCACCAAATGCTAATCCAGCTGAATGGATGTTGGAAGTTGTAGGTG |
| | CTGCTCCAGCTTCTCATGCTAACAAGGATTATCATCAAGTTTGGAAGGAC |
| | TCCAAAGAGTACCAAGAAATTCAATGCGAATTGAACAGGTTGGAGAGGGA |
| | ATTGAAAGATCATGGTAATGAGGATAATGAAGAGGGCCATAAGTCTTACG |
| | CTACCGATATTTTCTCTCAGATCGTTATCGTTTCCCACAGGTTCTTTCAAC |
| | ACTATTGGAGATCACCAAGATACTTGTGGCCAAAGTTGTTTTTGACTGCCT |
| | TCAACGAAATTTTCATCGGCTTCACTTTCTTCAAAGAGAAGCGTTCTTTAC |
| | AGGGTGTTCAAAACCAGATGTTGTCTACCTTTGTCTTTTGCGTTATCTTCA |
| | ACCCAATCTTGCAACAATTCTTGCCAGTTTACGTCGAACAAAGAAACTTGT |
| | ACGAAGCAAGAGAAAGACCATCTAGAACCTTTTCTTGGTTTACCTTCATCG |
| | TGTCCCAAATCATCGTTGAAATTCCTTGGAACTTTTTGGCTGGTACTATTG |
| | CCTTTTTCGTCTACTATTATCCCGTGGGTTTTTACAGAAATGCTTCCGAAG |
| | CTAATCAGTTGCACGAAAGAGGTGCATTATACTGGTTGTTCTGTACAGCA |
| | TTTTTCGTTTGGGTTGGTTCCATGGCTATTTTGGCTAACTCTTTTGTTGAAT |
| | ACGCTGCTGAAGCTTCTAACTTGGCTTTATTGTGTTTCGCTTTCTCTTTGG |
| | CTTTCAACGGTGTTTTAGTTCCACCAAACAAAATGCCTAGGTTTTGGATCT |
| | TCATGCACAGAGTTTCTCCATTGACCTACTACATTGATTCCGCTTTGTCTG |
| | TCGGTATGGCAAATGTTGGTGTAAAATGTTCTGCCTACGAATACGTTCAAT |
| | TCAAGCCACCAGTTAATCAAAACTGCGGTCAATACTTGAACAGCTACATTA |
| | ACTCTACTGGTACTGGTTACTTGGCTAATCCAAACGCTACTGATCAATGTT |
| | CTTTCTGCCCAATTTCCGAAACTAACGTTTACTTGGAAACTAGGGGCTCCT |
| | ATTACAAACATAGATGGCGTAACTACGGTATCTTCTTGTGTTTCATTGCCT |
| | TCGATTATGTTGCCGCCATTTTCTTATATTGGTTGGCTAGAGTTCCAAAGG |
| | GTAACAGAGTTAGCAAGAAAATCAAGTAA |
| SEQ ID | MDKDNENEIEEPNGYEVSSASGRYEGLDQTAAQSIKDLAQSLLEESRESIHA |
| NO: 982 | AGEGVNPVFMSESGDDYNPKVDPANDEFSSKEWIKNLSTIIKSDPSFYKPYT |
| | LGCSWRDLSAVGASADVAYQTTFENLPWKALTWVYRRLKAANESDTFQILK |
| | PMDGIVNPGELLVVLGRPGSGCTTLLKSISSNTHGFKVTEDSKISYSGLSPKDI |
| | KRHFRGEVVYNAESDIHLPHLTVYQTLLTVARLKTPQNRIPGVDRESWAKHV |
| | TEVAMATYGLSHTRNTKVGGDLVRGVSGGERKRVSIAEVTICGSKFQCWDN |
| | ATRGLDSATALEFIKALKAQAEIVDSAACVAIYQCSQDAYDLFDKVCVLYSGY |
| | QIYFGSAKNAKRYFQQMGYYCPSRQTTADFLTSITSPAERIINNGYIEKGINVP |
| | QTPEEMSDYWKNSPEYENLVRETDEFISQDHNSKISSIREAHQARQSKRARP |
| | AEPYTVSYLMQVKYLLIRNIWRIQNSYSITAFQVIGHSVMALLLGSMFYKVMK |
| | HTSTDTFYYRGSAMFFAVLFNAFSSLLEIFSLYEARPITEKHRTYSLYRPSADA |
| | FASILSEIPAKILTAIFFNLAYYFLVNFRRDGGRFFFYFLINIVGTFTMSHLFRCV |
| | GSLTKTLTEAMIPASILLLGMAMYAGFAIPETKMLGWSKWIWYINPLSYLFEAL |
| | MTNEFHDRKFPCATFIPSGGEYEQFRGKERICGVVGSVPGENYVLGDNFLK |
| | KSYNYDIEHKWRAFGVGMAYVIFFFFVYLFLCEINEGAKQKGEILVFPQTVVR |
| | KMRKQKKIRSRTNALNDLEKNIGTNATNLTDTTLVKESSDSTDEVQEQSGLT |
| | KSKAIFHWRNLCYDVQIKAETRRILSDVDGWVKPGTLTALMGSSGAGKTTLL |
| | DCLAERVTMGVITGDIFVNGKRRDASFPRSIGYCQQQDLHLKTATVRESLLFS |
| | AMLRQPKNVPTSEKKKYVEEVIKILEMEPYADAVVGVAGEGLNVEQRKRLTI |
| | GVELVAKPKLLVFLDEPTSGLDSQTAWSICQLMRKLVNRGQAILCTIHQPSAIL |
| | MQEFDRLLFLQKGGETVYFGELGDECSIMVDYFERNGAHKCPPNANPAEW |
| | MLEVVGAAPASHANKDYHQVWKDSKEYQEIQCELNRLERELKDHGNEDNE |
| | EGHKSYATDIFSQIVIVSHRFFQHYWRSPRYLWPKLFLTAFNEIFIGFTFFKEK |
| | RSLQGVQNQMLSTFVFCVIFNPILQQFLPVYVEQRNLYEARERPSRTFSWFT |
| | FIVSQIIVEIPWNFLAGTIAFFVYYYPVGFYRNASEANQLHERGALYWLFCTAF |
| | FVWVGSMAILANSFVEYAAEASNLALLCFAFSLAFNGVLVPPNKMPRFWIFM |
| | HRVSPLTYYIDSALSVGMANVGVKCSAYEYVQFKPPVNQNCGQYLNSYINST |
| | GTGYLANPNATDQCSFCPISETNVYLETRGSYYKHRWRNYGIFLCFIAFDYV |
| | AAIFLYWLARVPKGNRVSKKIK- |
| SEQ ID | ATGTCCGAGAAGCCAGATATTGCTGAAAACTCTGCTTCTATTGATGATGA |
| NO: 983 | CGCTTCCTCTTCTCAATCCAACTTGCATGAATACCATGGTTTCGATTACGA |
| | TGCCGAACAAAGAGTTAGAGATTTGGCTAGATCTTTGACCCATCAATCCG |
| | GTAACATTTCCACCTACTCTAATCAAGATGCTGCCACCGAATCTATTTTCA |
| | CTCCAGATATGGAAGGTATCAACCCAATCTTCACTAACAAAGAAGCTGAA |
| | GAGTACAACGAAAAGTTGGATCCTACTTCCGAAAACTTCTCATCTAAAGCT |
| | TGGGTTCAAAACATGGCTAACGTTGTTACTTCTGACCCAGAATTTTACAAG |
| | CCATACTCTTTGGGTTGTGTCTGGAAAAATTTGTCTGCTTCTGGTGAATCT |
| | TCCGATGTTGCTTATCAATCTACCGTTTTGAACATGCCCTACAAGTTGTTG |
| | AATTCCGCTTTTAGAAAAGCCAGATCTACCAAGACTGAAGATAGATTCCAA |
| | ATCTTGAAGCCAATGGATGGTTGTTTGAACCCTGGTGAATTATTGGTTGTT |
| | TTGGGTAGACCAGGTTCTGGTTGTACTACTTTGTTGAAGTCCATTTCCTCT |
| | AACACTCACGGTTTTGATGTTGGTGAAGATAGCGTTTTGTCTTACGCTGG |
| | TTTTACACCAGATGACATCAAAAAGCACTACAGAGGTGAAGTTGTTTACAA |
| | CGCTGAAGCCGATATTCATTTGCCACATTTGACTGTTTACGAAACCTTGTA |
| | CACCGTGTCTAGATTGAAAACTCCACAGAATAGAATCAAGGGTGTTGATA |
| | GAGATACCTTCGCTAGACATTTGACCGAAGTTGCTATGGCTACTTATGGT |
| | TTGTCTCATACCAGAAACACTAAGGTTGGTGATGATTTCGTTAGAGGTGTT |
| | TCAGGTGGTGAAAGAAAGAGAGTTTCAATTGCCGAAGTTTCCATTTGCGG |
| | TTCTAAGTTTCAATGTTGGGATAACGCTACTAGAGGTTTGGATTCTGCTAC |
| | TGCTTTGGAATTCATTAGAGCCTTGAAAACCCAAGCCACTATTGCTTCATC |
| | TGCTGCTACTGTTGCTATCTACCAATGTTCACAAGATGCCTACGATTTGTT |
| | CGATAAGGTTTGTGTTTTGGACGGTGGTTACCAAATCTACTTTGGTCCAG |
| | GTAATGAAGCCAAAAAGTACTTCGAAGATATGGGTTACAAGTGCCCAGAT |
| | AGACAAACTACTGCTGATTTCTTGACTTCTGTTACATCTCCAGCCGAAAGA |
| | ATCATTAACCCAGATTTCATTAAGAGGGGTATTGCCGTTCCACAAACTCCA |
| | AAGGATATGGGTGAATACTGGTTGAAGTCTCAAAACTACAAGGACCTGAT |
| | GAAGGAAATCGACCAAAAGTTGAACAACGACAACATCGAAGAATCTAGAA |
| | CCGCTGTAAAAGAAGCTCATATTGCCAAGCAATCTAAGAGAGCTAGACCA |
| | TCTTCTCCATACACTGTTTCTTATATGTTGCAGGTCAAGTACCTGCTGACT |
| | AGAAACTTTTGGAGAATTAGAAACAACGCCGGTGTGTCCTTGTTCATGATT |
| | ATTGGTAATTCTGCCATGGCCTTCATCTTGGGTTCTATGTTTTACAAGGTT |
| | ATGAAGAAGGGTGACACCTCTACTTTTTACTTTAGAGGTGCTGCTATGTTC |
| | TTCGCCGTTTTGTTTAATGCTTTCTCGTCCTTGTTGGAGATCTTCACATTAT |
| | ATGAAGCCAGACCAATTACCGAAAAGCACAGAACTTACAGCTTGTATCAT |
| | CCATCTGCTGATGCTTTGGCTTCTGTTTTCTCTGAATTGCCAACTAAGTGC |
| | ATTATCGCTGTTTGCTTCAACATCATCTTCTATTTCTTGGTCGACTTCAAGA |
| | GAAACGGCGATACTTTCTTTTTCTACCTGTTGATGAACGTCTTGGGTGTCT |
| | TGTCTATGTCTCATTTGTTTAGATGCGTTGGCTCTTTGACTAAGACTTTGT |
| | CTGAAGCTATGGTTCCAGCTTCTATGTTGTTGTTGGCTTTGTCAATGTTTA |
| | CCGGTTTCGCTATTCCAAAGACCAAAATGTTAGGTTGGTCTGAATGGATC |
| | TGGTACATCAATCCATTGTCCTACTTGTTCGAGTCCTTGATGATTAACGAA |
| | TTCCACGGTAGAAGATTCGCTTGTGCTCAATTTGTTCCATTTGGTCCTGCT |
| | TACGCTAACATTAACGGTACAAACAGAATTTGCTCTACCGTTGGTGCTGTA |
| | GCTGGTCAAGATTATGTTTTAGGTGACGACTTCGTCAAAGAGTCTTACGG |
| | TTACGAACACAAACATAAGTGGCGTTCTTTAGGTATTGGTTTGGCCTACGT |
| | TATCTTCTTCTTGTTCTTGTACTTGGTCTTGTGCGAATTCAATGGTGGTGC |
| | TAAACAAAAGGGTGAGATTTTGGTTTTCCCACAGGGTATTATCCGTAAGAT |
| | GAAGAAGCAAGGTAAGATCCAAGAAAAGAAAGCTGCCGGTGATATTGAAA |
| | ATGCTGGTGGTTCAAATGTTTCCGACAAGCAATTATTGAACGATACCTCC |
| | GAAGATTCCGAGGATTCTAATTCAGGTGTTGGTATCTCAAAGTCCGAAGC |
| | CATTTTTCATTGGAGAAACTTGTGTTACGACGTCCAAATCAAAACTGAAAC |
| | CAGAAGGATCCTGAACAACGTTGATGGTTGGGTTAAGCCAGGTACTTTGA |
| | CTGCTTTGATGGGTGCTTCAGGTGCAGGTAAAACTACTTTATTGGATTGC |
| | TTAGCTGAAAGGGTTACCATGGGTGTTATTACTGGTGAAGTTTCTGTCAAT |
| | GGCAGATTGAGAGATGAATCTTTCCCAAGATCTATCGGTTACTGTCAACA |
| | ACAAGACTTGCACTTGAAAACATCTACCGTCAGAGAATCCTTGAGATTCTC |
| | TGCTTATTTGAGACAACCATCGGATGTTTCCATTGAAGAGAAGAACAAGTA |
| | CGTCGAGGAAATCATTAAGATCTTGGAGATGGAAAAGTACGCTGATGCTG |
| | TTGTTGGTGTTGCTGGTGAAGGTTTGAATGTTGAACAGAGAAAAAGATTG |
| | ACCATCGGTGTTGAATTGGCTGCTAAACCTAAGTTGTTGGTCTTTTTGGAT |
| | GAACCTACATCTGGTTTGGATAGTCAAACTGCTTGGTCTATTTGCCAGCT |
| | GATGAAGAAACTAGCTGATCATGGTCAAGCTATTTTGTGCACTATTCATCA |
| | ACCATCCGCCATCTTGATGCAAGAATTTGATAGGTTGTTGTTCATGCAGA |
| | GAGGTGGTAAAACTGTTTACTTTGGTGACTTAGGTAAGGGTTGCCAAACC |
| | ATGATTGATTACTTTGAAAGAAACGGCTCTCATAAGTGTCCACCAGATGCT |
| | AATCCTGCAGAATGGATGTTGGAAGTTGTCGGTGCTGCTCCAGGTTCTCA |
| | TGCAAATCAAGATTACTATGAAGTCTGGCGTAATTCCGCTGAGTACAAAG |
| | CTGTTCATGAAGAATTGGAATGGATGGCTACTGAGTTGCCAAAGAAATCT |
| | CCAGAAACAAGTGCTGACGAACAACATGAATTCGCTACTTCTATCTTGTAC |
| | CAGTCTAAGTTGGTTTGTCGTAGATTGGGTGAACAGTATTGGAGATCACC |
| | AGAATATTTGTGGTCCAAGTTCATCCTGACTATCTTTAACCAGTTGTTCAT |
| | CGGTTTCACTTTCTTCAAGGCTGATACTTCTTTACAGGGCTTGCAAAATCA |
| | AATGTTGGCCATTTTCATGTTCACCGTCATCTTCAATCCTATCTTGCAACA |
| | ATACTTGCCAACCTTTGTTCAACAGAGAGACTTGTACGAAGCTAGAGAAA |
| | GACCATCTAGAACTTTTTCTTGGCTGGCTTTCATTATCTCCCAAATCGTTG |
| | TTGAAATCCCCTGGAATTTGTTGGCTGGTACAATTGCTTACTTTATCTACT |
| | ACTACCCAATCGGCTTTTACAGAAATGCTTCTGAAGCAGGTCAATTGCAC |
| | GAAAGAGGTGCTTTGTTTTGGTTGTTCTCTTGTGCTTACTACGTCTACATT |
| | GGTTCTATGGGTTTGATGTGCATCAGCTTCAACGAAATTGCAGAAAATGC |
| | AGCTAACACCGCTTCTTTGATGTTTACTATGGCCTTGTCTTTCTGTGGTGT |
| | TATGACTACTCCATCTAACATGCCAAGATTCTGGATCTTCATGTACAGAGT |
| | TTCACCTTTGACCTACTTGATTGATGCCTTGTTGTCTGTTGGTGTCGCTAA |
| | TGTTGATGCTCATTGCTCTGATTATGAGTTGTTGAGATTTGCTCCAGCTAA |
| | CGGTATGACTTGTGGTGAGTATATGGCTCCTTACATTCAATCTGTTGGTAC |
| | TGGTTACTTGAAGGATTCTTCTGCTACAGATGAATGTGCTTTCTGTACTGT |
| | TTCTGACACCAATTCTTACTTGGCCTCTGTTTCATCTCATTACAAGAATAG |
| | ATGGCGTAACTTCGGTATCTTCATTTGCTTTATTGCCTTCAACTACATGGC |
| | CGGTATTCTGTTTTATTACTTGGCTAGAGTTCCCAAGAAAGGTAACGGTTT |
| | GTTTTCGAAGTTCAAGAAGTAA |
| SEQ ID | MSEKPDIAENSASIDDDASSSQSNLHEYHGFDYDAEQRVRDLARSLTHQSG |
| NO: 984 | NISTYSNQDAATESIFTPDMEGINPIFTNKEAEEYNEKLDPTSENFSSKAWVQ |
| | NMANVVTSDPEFYKPYSLGCVWKNLSASGESSDVAYQSTVLNMPYKLLNSA |
| | FRKARSTKTEDRFQILKPMDGCLNPGELLVVLGRPGSGCTTLLKSISSNTHGF |
| | DVGEDSVLSYAGFTPDDIKKHYRGEVVYNAEADIHLPHLTVYETLYTVSRLKT |
| | PQNRIKGVDRDTFARHLTEVAMATYGLSHTRNTKVGDDFVRGVSGGERKRV |
| | SIAEVSICGSKFQCWDNATRGLDSATALEFIRALKTQATIASSAATVAIYQCSQ |
| | DAYDLFDKVCVLDGGYQIYFGPGNEAKKYFEDMGYKCPDRQTTADFLTSVT |
| | SPAERIINPDFIKRGIAVPQTPKDMGEYWLKSQNYKDLMKEIDQKLNNDNIEE |
| | SRTAVKEAHIAKQSKRARPSSPYTVSYMLQVKYLLTRNFWRIRNNAGVSLFM |
| | IIGNSAMAFILGSMFYKVMKKGDTSTFYFRGAAMFFAVLFNAFSSLLEIFTLYE |
| | ARPITEKHRTYSLYHPSADALASVFSELPTKCIIAVCFNIIFYFLVDFKRNGDTF |
| | FFYLLMNVLGVLSMSHLFRCVGSLTKTLSEAMVPASMLLLALSMFTGFAIPKT |
| | KMLGWSEWIWYINPLSYLFESLMINEFHGRRFACAQFVPFGPAYANINGTNRI |
| | CSTVGAVAGQDYVLGDDFVKESYGYEHKHKWRSLGIGLAYVIFFLFLYLVLC |
| | EFNGGAKQKGEILVFPQGIIRKMKKQGKIQEKKAAGDIENAGGSNVSDKQLL |
| | NDTSEDSEDSNSGVGISKSEAIFHWRNLCYDVQIKTETRRILNNVDGWVKPG |
| | TLTALMGASGAGKTTLLDCLAERVTMGVITGEVSVNGRLRDESFPRSIGYCQ |
| | QQDLHLKTSTVRESLRFSAYLRQPSDVSIEEKNKYVEEIIKILEMEKYADAVVG |
| | VAGEGLNVEQRKRLTIGVELAAKPKLLVFLDEPTSGLDSQTAWSICQLMKKLA |
| | DHGQAILCTIHQPSAILMQEFDRLLFMQRGGKTVYFGDLGKGCQTMIDYFER |
| | NGSHKCPPDANPAEWMLEVVGAAPGSHANQDYYEVWRNSAEYKAVHEELE |
| | WMATELPKKSPETSADEQHEFATSILYQSKLVCRRLGEQYWRSPEYLWSKFI |
| | LTIFNQLFIGFTFFKADTSLQGLQNQMLAIFMFTVIFNPILQQYLPTFVQQRDLY |
| | EARERPSRTFSWLAFIISQIVVEIPWNLLAGTIAYFIYYYPIGFYRNASEAGQLH |
| | ERGALFWLFSCAYYVYIGSMGLMCISFNEIAENAANTASLMFTMALSFCGVM |
| | TTPSNMPRFWIFMYRVSPLTYLIDALLSVGVANVDAHCSDYELLRFAPANGM |
| | TCGEYMAPYIQSVGTGYLKDSSATDECAFCTVSDTNSYLASVSSHYKNRWR |
| | NFGIFICFIAFNYMAGILFYYLARVPKKGNGLFSKFKK- |
| SEQ ID | ATGACCGAGAACATCCACTTGGGTGATCAAGTTTCTAAAACCTCTGCTCAATCTGTC |
| NO: 985 | GAAGAGTACAAGGGTTTTGATTCCAACGTTGATGATAACATCCAACACTTGGCTAG |
| | AAAGTTGACTAACGCTTCTCAATCTTCTTTGCCAGCTGCTGCTGGTGAAGATCAACA |
| | ATATTACCAAAACAACAACGACTTGGAAGCCCAACAATCTTTGTCTAGAGTTTCTAC |
| | TATTGCCCCAGGTGTTGTTGCTATTAACAACCCAGATATTGATCCAAGATTGGACCC |
| | AAACTCTGACCAATTCAATTCTAGATTCTGGGTCAAGAACTTCAAGAACCTGATGGA |
| | TAAGGATCCAGAACACTACAAGTCTTACTCATTGGGTATTGCCTACAAGAACTTGAG |
| | AGCTACTGGTGAAGCAGCTGGTGCTGATTATCAAACTACTGTTATGAATGCCCCATT |
| | GAAGTACGCTAATTTGGCTAAGAAGGCTTTCTTCACTTCCAAGGCTAAGAAAGAAG |
| | CTGGTAGATTCGATATCTTGAAGTCCATGGATGCTTTGGTTAGACCAGGTGAAGTT |
| | GTTGTTGTCTTAGGTAGACCTGGTTCTGGTTGTTCTACTCTGTTGAAAACTATTGCCT |
| | CTAACACTCATGGTTTCGCCATTGGTGAAGAGGCTGAAATTTCTTATGAAGGCTTGT |
| | CCCCAAAGGATATCAGAAAACATTATAGAGGTGAGGTTGTCTACAACGCCGAATCT |
| | GATATTCATTTCCCACATTTGACTGTCTGGCAAACTTTGTCTACTGTTGCTAAATTCA |
| | GAACCCCACAAAACAGAATTCCAGGTATCTCAAGAGAAGATTACGCTAACCATTTG |
| | ACCGAAGTTTACATGGCTACTTATGGTTTGTCTCATACCAAGAACACTAAGGTCGGT |
| | AACGAAAATGTTAGAGGTGTTTCTGGTGGTGAAAGAAAGAGAGTTTCAATTGCCGA |
| | AGTTTCTTTGTCTGGTGCTAGATTGCAATGTTGGGATAATGCTACTAGAGGTTTGGA |
| | TGCTGCTACTGCTTTGGAATTCATTAGAGCTTTGAGAACCCAAGCTGATGTTTTGGA |
| | TACAACTGCTTTCGTTGCTATCTACCAATGTTCTCAAGATGCCTACGATTTGTTCGAT |
| | AAGGTTACTGTCTTGTACGAAGGTCACCAAATCTATTTTGGTAGAGGTGATGAAGC |
| | CAGGGAATACTTTATTAAGATGGGTTGGTATTGCCCTCAAAGACAAACTACAGCTG |
| | ATTTCTTGACTTCTGTTACCTCTCCAAGAGAAAGAGTTCCACAAGAAGGTTTCGAAA |
| | ACAAGGTTCCAAAAACTCCACAAGAGTTCGAAACTTACTGGAAGAACTCTCCAGAA |
| | TACGCCAAGTTGATTAAGGATATCGACTCTGAATTCAAGCACCAACACGAACAAAA |
| | CTCTAAAGGTTTGGTCAAAGAAGCCCACAACAAGAAGCAAGCCAAACATATTAGAC |
| | CAACCTCTTCATACACTGTGTCTTTCTGGATGCAAACCAGATACTTGTTGACCAGAG |
| | ATTTCCAAAGAATCTGGAACGATTTCGGCTTCAACTCTTTTCAAGTTTTCGCCAATTC |
| | TTTCATGGCCCTGATTTTGTCCTCTATCTTTTACAACTTGCCAAAGACCACCGACTCA |
| | TTTTACTACAGAGGTGCTGCTATGTTTTTCGCCGTTTTGTTTAATGGCTTCTCGTCCTT |
| | CTTGGAAATCATGACTTTGTTTGAAGCCAGACCAATCATCGAAAAGCACAAGCAAT |
| | ACTCGTTGTATCATCCATCTGCTAACGCTTTGTCATCCGTTTTGTCTCAATTGCCAGC |
| | TAAGATCTTTACCTCCATTGCTTTCAACCTGGTCTTTTACTTCATGGTCAACTTCAGAA |
| | GAAACCCAGGTAGATTCTTCTTCTACTACTTGGTTAACTTGACCGCTACCTTCTCTAT |
| | GTCTCATTTGTTTAGATTGGTTGGTTCTGCTGCTACATCTTTACCAGAAGCTTTGGTT |
| | CCAGCTCAAGTTTTGTTGTTGGCTTTGACTATTTTCGTCGGTTTCACCATTCCAGTCA |
| | ACTATATGTTAGGTTGGTCCAGATGGATCAACTACTTGGATCCATTGGCTTATGCTT |
| | TCGAAGCTTTGATGGCAAATGAATTCGCTGGTGTTACTTACGATTGCTCATCTTTTGT |
| | TCCAGGTGACCCAAGATCTATTCCAAACATTCCATCTGATGGTTTCATCTGCAATGCT |
| | GTTGGTGCACAAACTGGTGAGTTTACTGTTGATGGTACTACCTATTTGGAAGTCGCT |
| | TACAAGTACAAGAATTCCCATAGATGGCGTAACTGGGGTATTACTTTAGCTTTTGCT |
| | TTGTTCTTCCTGGCCATCTACTTAGTTTTCTCTGAGTACAATGAATCCGCCATGCAAA |
| | AGGGTGAAGTCTTGTTATTTCAAAGGTCCACCTTGCGTAAGCTGAAGAAAGAAAAA |
| | GCTGCTTCCCAAAACGAATTGGAGTCTGGTAATGAGAAGGGTGTTGTTCCAAACGG |
| | TGAAGATGTTGATAAGGATGTTGATGTTATTCACGCTGGTACTCAAACTTTCCATTG |
| | GAGAGATGTTCATTACACCGTCAAGATCAAGAAAGAGGACAGAGAAATTTTGTCAG |
| | GTGTTGACGGTTGGGTTAAGCCAGGTACTTTGACTGCTTTAATGGGTGCTTCTGGT |
| | GCTGGTAAAACTACCTTGTTAGATGTCTTGGCTAACAGAGTTACTATGGGTGTTGTA |
| | ACTGGTGATATGTTCGTTAACGGTCACTTGAGAGATAACAGCTTCCAAAGATCTACT |
| | GGTTACGTTCAACAACAGGACTTGCATTTGAGAACTGCTACTGTTAGAGAAGCCTT |
| | GAAGTTTTCTGCTTACTTGAGACAACCAGCCTCTGTTTCTACTGCTGAAAAAGATCA |
| | ATACGTCGAAGAGGTCATCTCCATTTTGGATATGGAAAAGTATGCTGATGCCGTTGT |
| | TGGTGTTGCCGGTGAAGGTTTGAATGTTGAACAGAGAAAAAGATTGACCATCGGT |
| | GTTGAATTGGCTGCTAAACCTAAGCTGTTGTTATTCTTGGATGAACCTACCTCTGGT |
| | TTGGATTCTCAAACTGCTTGGTCTATTTGCCAGTTGATGAGAAAATTGGCTAACCAT |
| | GGTCAAGCCATTTTGTGTACTATTCATCAACCATCCGCCATCTTGATGCAAGAATTTG |
| | ATAGACTGTTGTTCTTGGCCAGAGGTGGTAAGACTGTTTATTTTGGTGATTTGGGTA |
| | AGAATTGCCAGACCTTGATTGACTACTTTGAAAAGTACGGTGCTCCAAAATGTCCAC |
| | CTGAAGCTAATCCAGCTGAATGGATGTTACATGTTATTGGTGCTGCTCCAGGTTCTC |
| | ATGCTAATCAAGATTATTACCAAGTCTGGTTGAACTCCACCGAAAGACAAGAAGTTA |
| | AGCAAGAATTGGACAGGATGGAAAGAGAATTGTCCCAATTACCAAGAGATGATTCC |
| | ATCGATCATAACGAATACGCTGCTCCATTCTGGAAACAATATGGTATCGTTACCCAA |
| | AGAGTGTTCCAACAGTATTGGAGATCACCAATCTACATCTACTCCAAGTTGTTCCTA |
| | GCTATCTCCTCTTCTATGTTTATCGGTTTTGCTTTCTTTAAGGCCAAGAATACCAGAC |
| | AAGGCTTGCAAAATCAAATGTTCGCTCTGTTCATGTTCCTGGTTATTTTCAACGCCTT |
| | GATCCAACAAACATTGCCTGAATATGTTAGACAGAGAGAGTTGTACGAGGTTAGAG |
| | AAAGACCATCTAAGACTTTTTCATGGAAGGCCTTTATTACCGCTCAAATCACATCTG |
| | AAGTTCCTTGGAATGCTCTGGTTGGTACTATTGCATTTTTGGTGTTTTATTACCCAGT |
| | CGGCTTCTACAACAATGCTGCTCCTAATGGTAGTGCTGAAGTTCATGATAGAGGTG |
| | CTTACGCTTGGTTTTTGACAGTTTTGTTTTTCGTCTACACCGGTTCTTTCGCCCATTTG |
| | GTTATTGCTCCATTGGAATTAGCTGATGCTGCAGGTAATTTGGCCTCTTTGATTTTCA |
| | CTTTGTGCTTGACTTTCTGCGGTGTTTTGGTTACATCAGAAGGTTTGCCAGGTTTCTG |
| | GATTTTCATGTATAGAGTCTCTCCATTCACCTACTTCATTGACGGTTTCTTGTCTAAT |
| | GCCGTTGCTCATAATGTTGTTAAGTGCTCCGATTCCGAATTGGTTCATTTTTCACCTC |
| | CACAAGGTGCTACTTGTGGTGATTATATGAAGGAATACTTGGAAAAGGCTGGTACA |
| | GGTTATGTTGAAGATCCATCTTCTACATCTGAATGCGGTTTCTGCTCTATTTCTTCTA |
| | CAGATGCCTTTTTGAAGGTCGTCAAATTGGATTATGGTAGACGTTGGAGAAACGTC |
| | GGTATTTTCATTGCCTTCATCGTTATCAACTGGATCTTGGCCGTTTTCTTTTATTGGTT |
| | GGCTAGAGTCCCAAAGAAGAACGATAGAGTTAAGTCAGGTGCTGAAGCTGAATCT |
| | ACAAACAAGACCGTTCAAAAGCAAGAAACTGCCTCCATTATCGAGAAAGAAGAGTC |
| | ATCTTCTAACTCCAAGGCTTAA |
| SEQ ID | MTENIHLGDQVSKTSAQSVEEYKGFDSNVDDNIQHLARKLTNASQSSLPAAAGEDQQ |
| NO: 986 | YYQNNNDLEAQQSLSRVSTIAPGVVAINNPDIDPRLDPNSDQFNSRFWVKNFKNLMD |
| | KDPEHYKSYSLGIAYKNLRATGEAAGADYQTTVMNAPLKYANLAKKAFFTSKAKKEAGR |
| | FDILKSMDALVRPGEVVVVLGRPGSGCSTLLKTIASNTHGFAIGEEAEISYEGLSPKDIRK |
| | HYRGEVVYNAESDIHFPHLTVWQTLSTVAKFRTPQNRIPGISREDYANHLTEVYMATYG |
| | LSHTKNTKVGNENVRGVSGGERKRVSIAEVSLSGARLQCWDNATRGLDAATALEFIRAL |
| | RTQADVLDTTAFVAIYQCSQDAYDLFDKVTVLYEGHQIYFGRGDEAREYFIKMGWYCP |
| | QRQTTADFLTSVTSPRERVPQEGFENKVPKTPQEFETYWKNSPEYAKLIKDIDSEFKHQ |
| | HEQNSKGLVKEAHNKKQAKHIRPTSSYTVSFWMQTRYLLTRDFQRIWNDFGFNSFQV |
| | FANSFMALILSSIFYNLPKTTDSFYYRGAAMFFAVLFNGFSSFLEIMTLFEARPIIEKHKQY |
| | SLYHPSANALSSVLSQLPAKIFTSIAFNLVFYFMVNFRRNPGRFFFYYLVNLTATFSMSHL |
| | FRLVGSAATSLPEALVPAQVLLLALTIFVGFTIPVNYMLGWSRWINYLDPLAYAFEALMA |
| | NEFAGVTYDCSSFVPGDPRSIPNIPSDGFICNAVGAQTGEFTVDGTTYLEVAYKYKNSHR |
| | WRNWGITLAFALFFLAIYLVFSEYNESAMQKGEVLLFQRSTLRKLKKEKAASQNELESGN |
| | EKGVVPNGEDVDKDVDVIHAGTQTFHWRDVHYTVKIKKEDREILSGVDGWVKPGTLT |
| | ALMGASGAGKTTLLDVLANRVTMGVVTGDMFVNGHLRDNSFQRSTGYVQQQDLHL |
| | RTATVREALKFSAYLRQPASVSTAEKDQYVEEVISILDMEKYADAVVGVAGEGLNVEQR |
| | KRLTIGVELAAKPKLLLFLDEPTSGLDSQTAWSICQLMRKLANHGQAILCTIHQPSAILM |
| | QEFDRLLFLARGGKTVYFGDLGKNCQTLIDYFEKYGAPKCPPEANPAEWMLHVIGAAP |
| | GSHANQDYYQVWLNSTERQEVKQELDRMERELSQLPRDDSIDHNEYAAPFWKQYGIV |
| | TQRVFQQYWRSPIYIYSKLFLAISSSMFIGFAFFKAKNTRQGLQNQMFALFMFLVIFNAL |
| | IQQTLPEYVRQRELYEVRERPSKTFSWKAFITAQITSEVPWNALVGTIAFLVFYYPVGFYN |
| | NAAPNGSAEVHDRGAYAWFLTVLFFVYTGSFAHLVIAPLELADAAGNLASLIFTLCLTFC |
| | GVLVTSEGLPGFWIFMYRVSPFTYFIDGFLSNAVAHNVVKCSDSELVHFSPPQGATCGD |
| | YMKEYLEKAGTGYVEDPSSTSECGFCSISSTDAFLKVVKLDYGRRWRNVGIFIAFIVINWI |
| | LAVFFYWLARVPKKNDRVKSGAEAESTNKTVQKQETASIIEKEESSSNSKA |
| SEQ ID | ATGGTTGATAACGTTGCTCCAAACGGTGCTTCTTACCAAAACAACAATATCGGTAAC |
| NO: 987 | GAAATGGCCGCCGATGATATTTCTGAACAAGCTTCTTCACCAGGTGTTTACAGAGGT |
| | TTTGAATCTGGTGCTCAACAATCCGTTAGAAACTTGGCTAGATCTTTGACTAACAGA |
| | TCTGGTGATGCTGTTTCTATGGCTGCTGATTGGAACACTATTAACCCAGTTTTCTCTA |
| | CCGTTGAGTCTCCATCTTATGATCCAAGATTGGACCCAAACTCTGACGAATTTTCTTC |
| | TTCTTTGTGGGTCCAAAACCTGTCTCAATTGGTTTCTTCTAACCCAGATCACTACAAG |
| | CCATATTCTTTGGGTTGTTCTTGGAGAAACTTGAGAGCTTACGGTAACTCTACTGAT |
| | GTTGCTTACCAATCCACTGTTGTTAACTTGCCATTGCAATTGGTTGAATACGTCTATA |
| | GAGCTGCTAGAAAAGCTAGACCAGAAGATACCTTCGATATCTTGAAACCTATGGAT |
| | GGTATGGTTAAGCCAGGTGAATTATTGGTTGTATTGGGTAGACCAGGTTCTGGTTG |
| | TTCTACTTTGTTGAAGTCTATCTCCTCTAACACCCATGGTTTCCATATCGACAAAGAA |
| | TCCGAAATCTCTTACGATGGCTTGTCCCCAAAAGAAATCTCTAGACATTATAGAGGT |
| | GAGGTTGTTTACAACGCTGAAGCTGATGTTCATTTCCCACATTTGTCTGTTTTCGACA |
| | CCTTGAAAACTGTTGCTAGATTAGCTTGTCCTACCAACAGAATCCAAGGTGTTGATA |
| | GAGAAACTTTCGCTACCCATATTACCGAAGTTGCTATGGCTACTTATGGTTTGTCTCA |
| | TACCAGAAACACTAAGGTGGGTAACGAATTGGTTAGAGGTGTTTCAGGTGGTGAA |
| | AGAAAGAGAGTTTCTATTGCCGAAGTCTCTATTTGCGGTTCTAAGTTTCAATGTTGG |
| | GATAACGCTACTAGAGGTTTGGATTCAGCTACTGCTTTGGAATTCATTAGAGCTTTG |
| | AGAACCCAAGCTAAGATGACTAGATCTTCTGCTGTTATTGCTATCTACCAATGCTCTC |
| | AAGATGCCTACGATTTGTTCGATAAGGTTTCTGTTTTACACGAGGGTTACCAAATCT |
| | ACTTCGGTAGAGCTAAAGAAGCCAAGCAATACTTCCAAGATATGGGTTATGTTTGC |
| | CCAGATAGACAAACTACTGCTGATTTCTTGACTGCTGTTACTAATCCAGCCGAAAGA |
| | ATCTTGAATGAGGATATGGTAAAGGCCGGTAAGAAGATTCCATTAACTGCTGCTGA |
| | AATGGAAGCTCATTGGAAACAATCTGAAGCTTACAAGGGTTTGTTGCAAGAAATCG |
| | ATTACTACTCTACCCATGATCAAGCCAACAACAGACAACAATTGAAAGAAGCTCACG |
| | TCGCTAAGCAATCTAAAAAGGCTAGAGCACAATCTCCATACACTGTCTCTTACGGTT |
| | TACAGGTTAAGTACCTGTTGATCAGAAACATGCAGAGGATTAGAAACAACATGGGT |
| | ATCACCTTGTTCCAAGTTATTGGTAATGGTGGCATGTCCTTCATTTTGGGTTCTATGT |
| | TTTACAAGGCTTTGAAGCACGATGATACCGCTGGTTTTTATTCTAGAGCTGGTGCTT |
| | TGTTTTTCGCCGTTTTGTTTAATGCTTTCAGCTGCATGTTGGAAATCTTGGCATTATA |
| | TGAAGCCAGGCCAATCTCCGAAAAACACAAGAGATATTCCTTGTACCATCCTTCTGC |
| | TGATGCTATGGCATCTGTTATTTCAGAAATTCCAGCTAAGCTGTTGACCTCTTTGACT |
| | TTCAATTTGGCTATGTACTTCCTGTGCAACTTTAAAAGAGAAGCTGGCGCTTTCTTCT |
| | TTTACTTCTTGATGACTATGGTTGGCACCTTTGCTATGTCCCATATTTTCAGATGTTTA |
| | GGTGCTTCCACTAAGACCTATGCTGAATCTATGGTTCCAGCTTCTTTGTTGTTGTTAG |
| | CTTTGGCTATCTACACCGGTTTTGCTATTCCAAAGACCAAGATTTTAGGTTGGGCTA |
| | AATGGATCTGGTACATCAATCCATTGTCCTACATCTTCGAATCCTTGATGGTTAACG |
| | AATTCCACGGTAGACAATTCAAGTGCTCTCAATTTGTTCCAGCTGGTCCAGGTTACG |
| | AAAATGTTAGTGGTACTCAAAGAGTTTGCTCTGCTGTTGGTGCTTTACCAGGTGAAG |
| | ATTACGTTTCTGGTGAAAGGTATATTAACGTCGCCTACAACTATTACCATTCTCATAA |
| | GTGGCGTGGTTTAGGTATTGGTTTGGCTTACGCTATTTTCTTTTTGGGTGTTTACTTG |
| | GCTGTCACCGAATTGAATGAATCTGCTAAACAAAGGGGTGAGATCTTGGTTTTTCCA |
| | CAAGCTGTTATGAGAAGGATGAAGAAGCAAAGAAAGTTAAGAGCTGGTACAAACG |
| | CTAACGGTTCCGATATTGAAAACACTGCTGGTGTTGCTACATTGAACGAAAAGAAG |
| | ATGCTGGAAGAGTCCTCTAATTCTGTTGCTTCATCTTCTTCTATGGGTGACGTTAAGT |
| | TGTCATCTTCTGAAGCTACTTTCCACTGGAAGAACGTTTGTTTTGAAGTCCCAATCAA |
| | GAAAGAAACCAGAAGGATCTTGGATAACGTAGATGGTTGGGTAAAACCAGGTACT |
| | TTGACTGCTTTGATGGGTGCTTCTGGTGCTGGTAAAACTACTTTATTGGACTGTTTG |
| | GCCTCTAGAGTTACTACTGGTACTATTACTGGTGACATGTTCATCAACGGTTTCTTGA |
| | GAGATGCTTCTTTCGCTAGATCTATTGGTTACTGTCAACAACAAGACTTGCACTTGC |
| | AAACTGCTACTGTTAGAGAATCTTTGAGATTCTCCGCTTACTTGAGACAACCATCTTC |
| | TGTTTCTAAGTCCGAGAAAGAAAAGTATGTTGAGGACGTGATTAAGATCTTGGAGA |
| | TGGAAACTTATGCTGATGCAGTTGTTGGTGTTGCCGGTGAAGGTTTGAATGTTGAA |
| | CAAAGAAAAAGGTTGACCATCGGTGTTGAATTGGCTGCTAAACCTAAGTTGCTGTT |
| | GTTTTTGGATGAACCTACATCTGGTTTGGACTCTCAAACTGCTTGGTCTATTTGTCAG |
| | TTGATGAGAAAGTTGGCTAACCATGGTCAAGCTATTTTGTGCACTATTCATCAACCA |
| | TCCGCTTTGTTGATGCAAGAATTTGATAGGTTGCTGTTCTTGCAAAGAGGTGGTAAG |
| | ACTGTTTACTTTGGTGACTTAGGTAAGGGTTGCCAAACCATGATTGATTACTTTGAA |
| | AAGAACGGTGCTCATCCATGTCCTGCTGGTGCTAATCCTGCTGAATGGATGTTGGA |
| | AGTTATAGGTGCTGCTCCAGGTTCTCATGCTAATCAAAATTACTCTGAAGTCTGGCG |
| | TAACTCCTCTGAGTACAAAGCTGTTCAAGAGGAATTGGAGTGGATGGAAAAAGAAT |
| | TGCCAAAAAGACCATTCGACAACTCCTCAGAACAAACTGAATTTGCTACCTCTTTGG |
| | TTTACCAGTACACTTTGGTTACCAAGAGATTGGTCGAACAATATTGGAGAACACCAT |
| | CTTACTTGTGGTCTAAGATTGGTTTGACCATCATCTCCCAAATCTTCATCGGTTTCAC |
| | TTTCTTCAAGGCCGATAATTCCATGCAAGGCTTGCAAAATCAAATGCTGTCCATCTTC |
| | ATGTTCTCCGTTATTTTCAATCCAACCTTGCAACAGTACTTGCCAACTTTTGTTGCTCA |
| | AAGAGACTTGTACGAAGCTAGAGAAAGACCATCTAGAACTTTTTCTTGGGTCGCTTT |
| | CATCTTGTCCCAAATTACTGTTGAAATCCCCTGGAACATTATTGCAGGTACTATAGG |
| | TTTCTTGGTCTACTATTATCCAGTCGGCTTGTATTCTACTGCTTCTAAAGCTGGTCAA |
| | TTGCACGAAAGAGGTGCTTTATTTTGGTTGTACGCTACAGCTTACTACGTTTTCACT |
| | GGTTCAATGGCTCAATTGTGTATTGCTGGTCAAGAAGTTGCTGAACCAGCAGGTCA |
| | AATGGCTAGTTTGTTGTTCACTCTGTCTTTGTCTTTCTGCGGTGTTTTGGTTGGTCCA |
| | TCATCAATGCCAGGTTTTTGGAAGTTTATGTACAGAGTCTCTCCATTGACCTACTTCG |
| | TTGATGGTACTTTGTCTACTGGTATTGCTAACGGTAAAGTTCAATGCTCCGATTACG |
| | AGTTGGTTAAGTTTAAACCAGCTAATGGTATGACCTGTGGTGAGTATATGGAACCTT |
| | ACATTAAGTTGGTTGGTACGGGTTATTTGTCTGATCCATCTGCTACTGATGAATGCA |
| | GATTTTGTTCCGTTTCTACCACCAACGATTACTTGTCAGCTGTTTCTTCATCATACTCT |
| | AGAAGGTGGCGTAATTATGGTATCTTCCTAGTGTACATTTTCTTCAACTTCGCTATG |
| | GCCGTTTTCATATATTGGTTGGCTAGAGTTCCTAAGAAGAGAAATAGAGTTGCTGAT |
| | GAACGTAAGACCGAAGCTCAAAAGTTAGTCGAAAAGTAA |
| SEQ ID | MVDNVAPNGASYQNNNIGNEMAADDISEQASSPGVYRGFESGAQQSVRNLARSLTN |
| NO: 988 | RSGDAVSMAADWNTINPVFSTVESPSYDPRLDPNSDEFSSSLWVQNLSQLVSSNPDHY |
| | KPYSLGCSWRNLRAYGNSTDVAYQSTVVNLPLQLVEYVYRAARKARPEDTFDILKPMD |
| | GMVKPGELLVVLGRPGSGCSTLLKSISSNTHGFHIDKESEISYDGLSPKEISRHYRGEVVY |
| | NAEADVHFPHLSVFDTLKTVARLACPTNRIQGVDRETFATHITEVAMATYGLSHTRNTK |
| | VGNELVRGVSGGERKRVSIAEVSICGSKFQCWDNATRGLDSATALEFIRALRTQAKMTR |
| | SSAVIAIYQCSQDAYDLFDKVSVLHEGYQIYFGRAKEAKQYFQDMGYVCPDRQTTADFL |
| | TAVTNPAERILNEDMVKAGKKIPLTAAEMEAHWKQSEAYKGLLQEIDYYSTHDQANN |
| | RQQLKEAHVAKQSKKARAQSPYTVSYGLQVKYLLIRNMQRIRNNMGITLFQVIGNGG |
| | MSFILGSMFYKALKHDDTAGFYSRAGALFFAVLFNAFSCMLEILALYEARPISEKHKRYSL |
| | YHPSADAMASVISEIPAKLLTSLTFNLAMYFLCNFKREAGAFFFYFLMTMVGTFAMSHIF |
| | RCLGASTKTYAESMVPASLLLLALAIYTGFAIPKTKILGWAKWIWYINPLSYIFESLMVNE |
| | FHGRQFKCSQFVPAGPGYENVSGTQRVCSAVGALPGEDYVSGERYINVAYNYYHSHK |
| | WRGLGIGLAYAIFFLGVYLAVTELNESAKQRGEILVFPQAVMRRMKKQRKLRAGTNAN |
| | GSDIENTAGVATLNEKKMLEESSNSVASSSSMGDVKLSSSEATFHWKNVCFEVPIKKET |
| | RRILDNVDGWVKPGTLTALMGASGAGKTTLLDCLASRVTTGTITGDMFINGFLRDASF |
| | ARSIGYCQQQDLHLQTATVRESLRFSAYLRQPSSVSKSEKEKYVEDVIKILEMETYADAV |
| | VGVAGEGLNVEQRKRLTIGVELAAKPKLLLFLDEPTSGLDSQTAWSICQLMRKLANHG |
| | QAILCTIHQPSALLMQEFDRLLFLQRGGKTVYFGDLGKGCQTMIDYFEKNGAHPCPAG |
| | ANPAEWMLEVIGAAPGSHANQNYSEVWRNSSEYKAVQEELEWMEKELPKRPFDNSS |
| | EQTEFATSLVYQYTLVTKRLVEQYWRTPSYLWSKIGLTIISQIFIGFTFFKADNSMQGLQN |
| | QMLSIFMFSVIFNPTLQQYLPTFVAQRDLYEARERPSRTFSWVAFILSQITVEIPWNIIAG |
| | TIGFLVYYYPVGLYSTASKAGQLHERGALFWLYATAYYVFTGSMAQLCIAGQEVAEPAG |
| | QMASLLFTLSLSFCGVLVGPSSMPGFWKFMYRVSPLTYFVDGTLSTGIANGKVQCSDYE |
| | LVKFKPANGMTCGEYMEPYIKLVGTGYLSDPSATDECRFCSVSTTNDYLSAVSSSYSRR |
| | WRNYGIFLVYIFFNFAMAVFIYWLARVPKKRNRVADERKTEAQKLVEK |
| SEQ ID | ATGTCCTTGGGTACTACCCATTTGCCAGAAGCTTTGGCTGCTAATCATGAAGCTGCT |
| NO: 989 | GCTGCTACTTCTAACAATTCTGAACCAGATTCTTTCCATCCAGAGTACAGAGGTTTTG |
| | AAGATCAAGCTCAAGATCACGTTAGAAACTTGGCTAGAACTTTGACTAATGCTTCTG |
| | CTCCAGGTTCTGTTGCTTCTATGTCTCCAATCAATCCAGTTTTCTCTACTCCAGATGCT |
| | CCACATTATGATGCTAGATTGGATCCTAACTCCGACGAATTTTCTTCTGCTTTTTGGG |
| | TTCACAACGTGTCTCAATTGGTTGCTAGAGATCCAGATCATTACAAGCCATATTCTTT |
| | GGGTTGTTCTTGGAGAAACTTGAGAGCTTATGGTAACGCTACTGATGTTGCTTACCA |
| | ATCTACTGTTGCTAACTTGCCATTGCAATTGGCTGAAACTGTTTTGAGAATGGCTAG |
| | AAAAGCTAGACCAGAAGATACCTTCGATATCTTGAAACCTATGGATGGTTTGGTTA |
| | AGCCAAGAGAGTTGTTGGTTGTTTTGGGTAGACCAGGTTCAGGTTGTTCTACTTTGT |
| | TGAAAACTGTTTCCGCTAACACCCATGGTTTTCATGTTGATCCAAAGTCTCAAATCTC |
| | CTACGATGGTTTGACTCCAAAAGATGTTGCTAGACATTACAGAGGTGAAGTTGTTTA |
| | CAACGCCGAATCTGATGTTCATTTCCCACATTTGACTGTTTTCGACACCTTGAAAACA |
| | GTTGCCAGATTGTCTTGTCCATCCAACAGAATTCATGGTGTTGATAGAGAAACCTTC |
| | TCTACCCATATTACCGAAGTTGCTATGGCTACTTATGGTTTGTCTCATACCAGAAACA |
| | CTAAGGTCGGTAACGAATTGGTTAGAGGTGTTTCAGGTGGTGAAAGAAAGAGAGT |
| | TTCTATTGCCGAAGTCTCTATTTGCGGTTCTAAGTTTCAATGTTGGGATAATGCTACC |
| | AGAGGTTTGGATTCTGCTACTGCTTTGGAATTCATTAGAGCTTTGAGAACCGAAGCT |
| | AAGTTGACTCATTCTGCTGCTGTTATTGCTATCTACCAATGTTCTCAAGATGCCTACG |
| | ATTTGTTCGATAAGGTTTCTGTTTTACACGAGGGTTATCAGATCTATTTCGGTCCAGC |
| | TAATCAAGCCAAGCAATACTTTGAAACTATGGGTTACGTTTGCCCATCTAGACAAAC |
| | TACTGCTGATTTCTTGACTGCTGTTACTAATCCAGCTGAAAGAATTGTCAGAGAAGG |
| | ATGTAGACCACCAGCTACTGCTGTTGAAATGGAAAAGTATTGGAAACAGTCCCCAG |
| | ATTACAAAAGGTTGTTGGGTGAAATTGATGAATACGCTGCTCATGAACAAGCCGAT |
| | AACATGAGAAATTTGGCCGAAAATCATGTTGCTAAGCAATCAAGAAGGGCTAGACC |
| | AAAATCTCCATACACTGTTTCTTACGGCTTGCAGGTTAAGTACTTGTTGATCAGAAA |
| | CATGCAGAGGATCAGATCCAATATGGGTGTCACTGTTTTCCAAGTTATTGGTAATGG |
| | TTCCATGGCCTTTATCTTGGGTTCTATGTTTTACAAGGTCTTGAAGCACGATACCACC |
| | GAAGGTTTTTATTCTAGAGCTGGTGCTTTGTTCTTCGCCGTTTTGTTTAATGCTTTCTC |
| | CTGCTTGTTGGAAATCTTCGCATTATATGAAGCCAGACCAGTTTCTGAAAAGCACAA |
| | GAGATACAGCTTGTATCATCCATCTGCTGATGCTATGGCATCCGTTATTTCTGAAGTT |
| | CCAGCTAAGTTATTGACCTCCGTTTCTTTTAACTTGGCCTTGTACTTCTTGTGCAACTT |
| | TAAAAGAGAAGCTGGCGCCTTTTTCTTCTACTTCTTGATGACTATGGTTGCCACGTTT |
| | TTGATGTCCCATATTTTCAGATGTTTGGGTGCTTCTACTAAGACCTATGCTGAATCTA |
| | TGGTTCCAGCTTCAGTTTTGTTGTTGGCTTTGTCTATCTACACCGGTTTTGCTATTCCA |
| | AAGACCAAGATTTTAGGTTGGTCTAAGTGGATCTGGTACATCAATCCATTGTCCTAC |
| | GTTTTCGAATCCTTGATGGTTAACGAATTCCACGATAGAAGATTCGATTGCTCTGCT |
| | TATATTCCAACTGGTCCAGCTTACGAATCTATCTCTGGTACTGAAAGAGTTTGTTCTG |
| | CTGTTGGTGCTGTTCCAGGTCAAGATTATGTTAATGGTGAAACCTACATCAACGTTG |
| | CCTATGGTTATTACCATTCTCATAAGTGGCGTGGTTTAGGTATTGGTTTGGCTTATG |
| | CTATAGTTTTCTTGGGTGTTTACTTGGCCGTTACCGAATTCAATGAATCCGCTAAACA |
| | AAGGGGCGAGATTTTAGTTTTTCCACAAGCTATCATGAGGCGTATGAAGAAGCAAA |
| | GACATAGGTCCGAAAAGAACAATGGTGGTGGTGCTAGAGGTTCTGAAACTACTGG |
| | TGGTGGCGGAGGTGGTGGTTCTGCTGCAGATTTGGAATCTTCTGCTGGTGTTGTTC |
| | CTGTTAACGAAAAGAAGCTATTGGAAGATTCTACCGATTCTGCCAATTCTACCAACT |
| | CTTCTATGGGTGAAGCTGGTTTAACTCAATCTGAAGCTATCTATCATTGGAGGAACG |
| | TTTGTTTCGATGTCCCCATTAAGAAAGAAACCAGAAGAATCTTGGATCACGTTGATG |
| | GTTGGGTAAAACCAGGTACTTTGACTGCTTTGATGGGTGCATCTGGTGCTGGTAAA |
| | ACTACTTTATTGGACTGTTTGGCTTCTAGAGTTACCACTGGTGTTATTACTGGTGACA |
| | TGTTCATTAACGGTTTCTTGAGAGATTCCTCATTCGCTAGATCTATTGGTTACTGTCA |
| | ACAACAAGACTTGCACTTGGAAACTTCTACCGTTAGAGAATCTTTGAGATTCTCCGC |
| | TTATTTGAGACAACCATCTCACATCTCTAAGCAAGAAAAGGATAGATACGTCGAAG |
| | AAGTCATCAGGATCTTGGAAATGTTGCCATACGCTGATGCTGTTGTTGGTGTTGCTG |
| | GTGAAGGTTTGAATGTTGAACAGAGAAAAAGATTGACCATCGGTGTTGAATTAGCT |
| | GCTAAACCTAAGCTGTTGCTGTTTTTGGATGAACCTACTTCCGGTTTAGATTCTCAAA |
| | CTGCTTGGTCTATTTGCCAGTTGATGAGAAAGTTAGCCAATCATGGTCAAGCTATTT |
| | TGTGCACCATTCATCAACCATCAGCTTTGTTGATGCAAGAATTCGATAGGTTGCTAT |
| | TCTTGCAAAGAGGTGGTAAGACTGTTTACTTTGGTGACTTAGGTCCAGGTTGCCAA |
| | ACTATGATTGATTACTTTGAATCTCATGGTGCCCATAAGTGTCCAGATGGTGCTAAT |
| | CCTGCAGAATGGATGTTGGAAGTTATAGGTGCTGCTCCTGGTACTCATGCTTCTCAA |
| | AATTATCATGACGTTTGGAGGAATTCCGATGAATATCGTGCTGTTCAAGAGGAATT |
| | GGAATGGATGGAACGTGAATTGCCAAAGAAGCCAATTGATACCTCTAACGAACAGT |
| | CTGAATTCTCGACTTCTTTGTTCTACCAATACTGTTTGGTCACCAAGCGTTTGTTGGA |
| | ACAATATTGGAGAACTCCATCTTACCTGTGGTCTAAAATTGGTTTGACCATCATCTCC |
| | CAAATCTTCATCGGTTTCACTTTCTTCAAGGCCGATTCTTCTATGCAAGGCTTGCAAA |
| | ATCAAATGCTGTCCATCTTCATGTTCACCGTTATTTTCAATCCAACCTTGCAACAATA |
| | CCTGCCAACTTTTGTTTCTCAGAGAGACTTGTACGAAGCTAGAGAAAGACCATCTAG |
| | AACATTTTCTTGGCTGGCCTTCATTTTGTCCCAGATTTCAGTTGAAATCCCCTGGAAT |
| | ATTTTGGCTGGTACTGTTGGTTTCTTCGTCTACTACTTTCCAGTCGGTTTTTACAACA |
| | ATGCTTCCTTCGCTGATCAATTGCACGAAAGAGGTGCTTTATTTTGGTTGTACGCTA |
| | CCGCTTTTTACGTTTTCACTGGTTCTATGGCCCAATTGGTTATTGCATCTCAAGAAGT |
| | TGCTCAATCCGCTGGTCAAATTTCCTCTTTGTTGTTCACTATGTGCTTGTCTTTCTGCG |
| | GTGTTATGGTTCAACCTAACAATATGCCTAGATTCTGGATTTTCATGTACCGTGTTTC |
| | TCCATTGACCTACTTCATAGATGGTACATTGTCTACCGGTTTGGCAAATGCAGATGT |
| | TCACTGTTCTGATTACGAGATGGTTAGTTTTACTCCACCACAAGGTCAAACTTGTGG |
| | TCAGTATATGCAATCCTACATTTCTTCAGCTGGTACAGGTTATTTGGCTGATCCAGAT |
| | GCAACTGATGAATGTAGATTCTGTTCTGTCTCCTCTACCAACGATTACTTGAAAGCA |
| | GTTTCTTCCGAGTATACACATAGATGGCGTAACTACGGTATCTTCCTAGCTTTCATTA |
| | TGTTCAATTTTGCTGCCGCCGTCTTTTTCTACTGGTTGGCTAGAGTTCCTAAGAAGA |
| | GAAATAGAGTTGCTGACGAAAGAAAACCCGAAGCTCAAAAGATTCAAGAGAAGTA |
| | A |
| SEQ ID | MSLGTTHLPEALAANHEAAAATSNNSEPDSFHPEYRGFEDQAQDHVRNLARTLTNAS |
| NO: 990 | APGSVASMSPINPVFSTPDAPHYDARLDPNSDEFSSAFWVHNVSQLVARDPDHYKPYS |
| | LGCSWRNLRAYGNATDVAYQSTVANLPLQLAETVLRMARKARPEDTFDILKPMDGLV |
| | KPRELLVVLGRPGSGCSTLLKTVSANTHGFHVDPKSQISYDGLTPKDVARHYRGEVVYN |
| | AESDVHFPHLTVFDTLKTVARLSCPSNRIHGVDRETFSTHITEVAMATYGLSHTRNTKVG |
| | NELVRGVSGGERKRVSIAEVSICGSKFQCWDNATRGLDSATALEFIRALRTEAKLTHSAA |
| | VIAIYQCSQDAYDLFDKVSVLHEGYQIYFGPANQAKQYFETMGYVCPSRQTTADFLTAV |
| | TNPAERIVREGCRPPATAVEMEKYWKQSPDYKRLLGEIDEYAAHEQADNMRNLAENH |
| | VAKQSRRARPKSPYTVSYGLQVKYLLIRNMQRIRSNMGVTVFQVIGNGSMAFILGSMF |
| | YKVLKHDTTEGFYSRAGALFFAVLFNAFSCLLEIFALYEARPVSEKHKRYSLYHPSADAMA |
| | SVISEVPAKLLTSVSFNLALYFLCNFKREAGAFFFYFLMTMVATFLMSHIFRCLGASTKTY |
| | AESMVPASVLLLALSIYTGFAIPKTKILGWSKWIWYINPLSYVFESLMVNEFHDRRFDCS |
| | AYIPTGPAYESISGTERVCSAVGAVPGQDYVNGETYINVAYGYYHSHKWRGLGIGLAYA |
| | IVFLGVYLAVTEFNESAKQRGEILVFPQAIMRRMKKQRHRSEKNNGGGARGSETTGGG |
| | GGGGSAADLESSAGVVPVNEKKLLEDSTDSANSTNSSMGEAGLTQSEAIYHWRNVCF |
| | DVPIKKETRRILDHVDGWVKPGTLTALMGASGAGKTTLLDCLASRVTTGVITGDMFING |
| | FLRDSSFARSIGYCQQQDLHLETSTVRESLRFSAYLRQPSHISKQEKDRYVEEVIRILEMLP |
| | YADAVVGVAGEGLNVEQRKRLTIGVELAAKPKLLLFLDEPTSGLDSQTAWSICQLMRKL |
| | ANHGQAILCTIHQPSALLMQEFDRLLFLQRGGKTVYFGDLGPGCQTMIDYFESHGAHK |
| | CPDGANPAEWMLEVIGAAPGTHASQNYHDVWRNSDEYRAVQEELEWMERELPKKPI |
| | DTSNEQSEFSTSLFYQYCLVTKRLLEQYWRTPSYLWSKIGLTIISQIFIGFTFFKADSSMQG |
| | LQNQMLSIFMFTVIFNPTLQQYLPTFVSQRDLYEARERPSRTFSWLAFILSQISVEIPWNI |
| | LAGTVGFFVYYFPVGFYNNASFADQLHERGALFWLYATAFYVFTGSMAQLVIASQEVA |
| | QSAGQISSLLFTMCLSFCGVMVQPNNMPRFWIFMYRVSPLTYFIDGTLSTGLANADVH |
| | CSDYEMVSFTPPQGQTCGQYMQSYISSAGTGYLADPDATDECRFCSVSSTNDYLKAVS |
| | SEYTHRWRNYGIFLAFIMFNFAAAVFFYWLARVPKKRNRVADERKPEAQKIQEK |
| SEQ ID | ATGTCCGACAACTCCGAAGTCGAATCTTTCAATGCTAAACATGTTGAAGCT |
| NO: 991 | TCCGGTGGTACTAAGGATCCATATACTGGTGTTCCAGATACCAACTCTGT |
| | TCAATCTTTGGCTAGAACTTTCACCCATATGTCTTTGGCTTCATCCTCTAA |
| | CGATAACGAAATCTATGGTGTTCCTGTTGATGGTCCAGAAGGTGTTGAAT |
| | CTTATAACGCTAAGATCGACCCAAACTCTGAAGAATTTGATGCTCATGCTT |
| | GGATGAGAAACTTGAACAGATTGAGATCTGCTGATCCAGACTACTACAAG |
| | AACATCTCTTTAGGTATGGCTTACAAGAACTTGTCTGCTTTCGGTGATTCT |
| | TCCGATGTTGTTTATCAACCTACCGTCTTGAACGTGTTCCAAAAATCCATT |
| | GAGGACATCTACAGAAAGGTTAGAAAAGCTAGACCATCCGATAAGTTCAC |
| | CATTTTGAAACCTATGGACGGTATTTTGAAGCCAGGTTCTTTGAATGTTGT |
| | TTTGGGTAAACCAGGTTCTGGTTGTTCTACTTTGTTGAAAACCTTGTCCTC |
| | TTCTACCTTCGGTTTCGAAGTTACCAAGGATTCCGTTATTTCCTACGATGG |
| | TATTACCCCAAAAGAGATCGAAAACAACTACAGAGGTGATGTTGTATACC |
| | AAGGTGAAGTTGATATTCACTTCCCACATTTGACTGTGTTCGAAACCTTGA |
| | ACAATGTTGCTTTGTTGACTACTCCAAAGAACAGGATTAAGGGTTTGTCCA |
| | GAGAAGAATTCTCCAAACATATGGCTGAAGCTACTATGGCTATGTATGGT |
| | TTGTCTCATACCAAGAACACTAAGGTCGGTAACGAATTGGTTAGAGGTGT |
| | TTCTGGTGGTGAAAGAAAAAGAGTTTCCATCTGCGAAATCTCATTGGTCA |
| | ATGGTAAGATCGTTTGCTACGATAACTCTACCAGAGGTTTGGATTCTGCTT |
| | CTACATTGTCCTTCATCAAGTCCTTGAAAACCCAATCTAAAGCTTCTGATA |
| | CCACTTCCGTTGTTGCTATCTATCAATGTTCTCAAGATGCCTACGATTTGT |
| | TCGATAACGTCATCGTTTTGGATGAAGGTTACCAGTTGTATAACGGTCCAT |
| | CTAACAAAGCCAAGGACTACTTTATTAAGATGGGTTACGTTTGCCCAGAA |
| | AGACAAACTACTGCTGATTTCTTGACTGCTGTTACTTCTCCAACTGAAAGA |
| | ATCAAGAATACCGAGATGATGGAAAAGGGTATCAAGATTCCAGAAACCTC |
| | CTTGGAAATGTACGAATACTGGAATGCTTCCGAAGAATGCCAAGAATTGA |
| | AGTCCGAAATCGACTACTACTTGTCCCATATCGATTCCTCATTGAAGGATC |
| | AATTCCATCAAGCTCATACTGCTTCTCAAGCTAAAAGAGCTAGACCTTCTT |
| | CTTCTTACTTGTTGACCTTTCAATTGCAGGTCAAGTATTTGTTGCAGAGAA |
| | ACTTCACTAGGATCAAGAAGGATATCGGCTTGTCTATGTTTCAAGTCTTGG |
| | GTAATTCTTTCATGGCCTTGATTATTGCCTCCATGTTCTACAAGGTTATGT |
| | ACTACACTAACACCTCCACGTTCTTCTATAGAGGTGGTACTTTGTTTTACG |
| | CCGTCTTGTTTAACTCCTTCTCCTCCTTGTTGGAAATCATGACATTATACG |
| | AAGCCAGGCCAATTATCGAGAAGCAAAAGAATTTGGCAATGTACCATCCA |
| | TCTGCTGAAGCCGTTTCTTCTATTTTGTCTGAAATGCCAGCCAAGATTATT |
| | ACCGCTATTGCCTTCAATATGTTCTACTACTGGATGACCAATTTGAAGAGA |
| | GATGCTGGCGCTTTCTTTTTCTACTTGCTGATGAATTTCGTTTGCCTGTTG |
| | GCTATGTCTCACATCTTTAGATTCATTGGTTCTGCCACTAAGTCTTTTCCA |
| | GGTGCTATGGTTCCAGCTTCTGTTATTTTGTTGGGTATTTCTATGTACGCC |
| | GGTTTCGCTATTCCAAAGACTTCTATGTTAGGTTGGTCTAAGTGGATCTAC |
| | TGGATTAACCCAATTCAATACGGTTTCGAGTCCTTGATGATCAACGAATTT |
| | CATGGTGTCGAATACCCATGCTCATCTTATATTCCATCTGGTACTGGTTAC |
| | TCCGATTTTGATTCAGCTTACAAAACCTGCTCTGTTGTTGGTGCTGTTCCA |
| | GGTTCAGATATTGTCTCTGGTGATTTGTTCCTGAAGTTGTCTTATGGTTAC |
| | GAACATTCTCATAAGTGGCGTGGTTTTGGTGTTGTTTTAGCTTACGCTATT |
| | TTCTTCTTCGGTGTCTACTTGACTTTCACCGAGTATAATGAATCCGCTAAG |
| | CAAAAGGGTGAGATCATTGTCTTTCCACAAGCTGTTGTTCGTAAGATCAA |
| | GAAGATGTCTAAGTCTACCCACGATTTGGAATCAGCTTCAGCATCTGATG |
| | AATCTTACACCGACAAAAAGTTGGTTTCCGATGATTACGATGAATCCCAAG |
| | ATTCCTACAACGATGTTGGTTTGAGTGAATCCGAAGCTACTTTACATTGGA |
| | GAGATTTGTGTTACGACGTCCAAATCAAAGGTGAAACTAGAAGGATCTTG |
| | AACAACGTAGATGGTTGGGTTGCTAAGAACTCTATTACTGCTTTGATGGG |
| | TTCTTCTGGTGCTGGTAAAACTACTTTGATGGATTGCTTGGCTTCTAGAGT |
| | TACCATGGGTGTTATTACCGGTGATATTTTGGTTAACGGTAGATTGAGAG |
| | ATGAGGGTTTCCCAAGATCTATTGGTTACTGTCAACAACAAGACTTGCATT |
| | TGGCTACTGCTACTGTTAGAGAATCCTTGAAATTCTCCGCTTACTTGAGAC |
| | AACCAGCCTCTGTTTCTAAAGAAGAGAAAGACGCTTACGTCGAATCCGTC |
| | ATTAAGATCTTGGATATGCAAAGATACGCTGATGCTGTTGTAGGTGTAATG |
| | GGTGAAGGTTTGAACGTTGAACAAAGAAAACGTTTGACCATCGGTGTAGA |
| | ATTGGCTGCTAAACCTAAGCTGTTGATGTTCTTAGATGAACCTACTTCTGG |
| | TTTGGACTCTCAAACTGCTTGGTCTATTTGTCAGTTGATGAGAAAGTTGGC |
| | TAACCATGGTCAAGCTATTTTGTGCACTATTCATCAACCATCCGCCATGTT |
| | GATTGATCAGTTTGATAGGTTGCTGTTCTTGCAGAAAGGTGGTAAGACTG |
| | CTTATTTTGGTGACTTAGGTGAGGGTTGTAAGACCATGATTGATTACTTTG |
| | AATCTAAGGGTGCTCCAAAGTGTCCACCAAATGCTAATCCAGCTGAATGG |
| | ATGTTGGATATTGTCCAAGCTAGAGATTATCATGAAGCCTGGAAATCTTCA |
| | GACGAGTACAAACAAGTTCATGCTACTTTGGACGAAATGGAAAGAGAATT |
| | GCCAAACATCGTTATCTCCAACGCTGATGATACTTCTGCTTTTGCTGCTTC |
| | ATTTCCAGTCCAACTGTTTTACGTTTACAAGAGAGTCGTCCAACAATATTG |
| | GAGAACTCCAATCTATTTGTGGGCCAAGTTTTTCGTTACTGGTGCTTCTGA |
| | GTTGTTCATCGGTTTTACTTTCTTTAAGGCCAACCACACATTGCAGGGTTT |
| | GAAGAATCAAATGTTGGCCATCTTCATGTTCGTCGTCATTTTTAACCCATT |
| | CTTGCAACAGTACTTGCCAATCTTTACTCAACAGAGAGACTTGTACGAAG |
| | CTAGGGAAAGACCATCTAGAACATTTTCTTGGTACGCTTTCTTGATCGGTC |
| | AAATGTTAGCTGAAATCTTCCCAAACATTTTGTGTGCCATCTTGGGCTTTT |
| | TCTGTTTTTACTACCCAATTGGTTTCGCCTCTAACGCTTCTTATTCTGGTC |
| | AATTGGTTGAGAAGTCTGGCCTGTTTTTCTTCTACTCCTTGATTTTCTTCA |
| | CCTGGATTGGTTCTACAGCATTGATGGTTGCAGCTCCATTTGAAGATCCT |
| | CAAGCTGGTGGTCATTTGGCCAATTTGATGTTTACAATGGCCTTGTCTTTC |
| | AACGGTGTTTTTGTTGGTCCAGGTAAATTGCCAGGTTTTTGGAAGTTTATG |
| | TACCGTGTTTCTCCATTGACCTACTTCGTTGATGGTGCTTTGTCTATAGGT |
| | TTGTCCGATAACAAGGTTGAATGCTCTCAATACGAGTACACTGATGTTCAA |
| | TTGCCAGCTGGTATGACTTGTGGTGAATATTTGTCTCCTTACATCGAGAA |
| | GGTTGGTACAGGTTACATTTTGGATAAGAACGCTACCTCTGAATGCAAGA |
| | TGTGTCAAATTTCTACTACCAACGCCTTCTTGACCACTGTTTCATCTAAAT |
| | ATTCTAGGCGTTGGCGTAACCTGGGTATTTTCATTGCTTACATTGCTTTCA |
| | ACTACGTGATGGCCATGTTCTTATATTGGTGGGCTAGAGTTCCAAAGAAG |
| | GCTAACAGAGTTTCTGACGAAAAAGACTCTTCCAAGGATAAGAAGGACGA |
| | GAAGAAGTAG |
| SEQ ID | MSDNSEVESFNAKHVEASGGTKDPYTGVPDTNSVQSLARTFTHMSLASSSN |
| NO: 992 | DNEIYGVPVDGPEGVESYNAKIDPNSEEFDAHAWMRNLNRLRSADPDYYKNI |
| | SLGMAYKNLSAFGDSSDVVYQPTVLNVFQKSIEDIYRKVRKARPSDKFTILKP |
| | MDGILKPGSLNVVLGKPGSGCSTLLKTLSSSTFGFEVTKDSVISYDGITPKEIE |
| | NNYRGDVVYQGEVDIHFPHLTVFETLNNVALLTTPKNRIKGLSREEFSKHMA |
| | EATMAMYGLSHTKNTKVGNELVRGVSGGERKRVSICEISLVNGKIVCYDNST |
| | RGLDSASTLSFIKSLKTQSKASDTTSVVAIYQCSQDAYDLFDNVIVLDEGYQL |
| | YNGPSNKAKDYFIKMGYVCPERQTTADFLTAVTSPTERIKNTEMMEKGIKIPE |
| | TSLEMYEYWNASEECQELKSEIDYYLSHIDSSLKDQFHQAHTASQAKRARPS |
| | SSYLLTFQLQVKYLLQRNFTRIKNDIGLSMFQVLGNSFMALIIASMFYKVMYYT |
| | NTSTFFYRGGTLFYAVLFNSFSSLLEIMTLYEARPIIEKQKNLAMYHPSAEAVS |
| | SILSEMPAKIITAIAFNMFYYWMTNLKRDAGAFFFYLLMNFVCLLAMSHIFRFIG |
| | SATKSFPGAMVPASVILLGISMYAGFAIPKTSMLGWSKWIYWINPIQYGFESL |
| | MINEFHGVEYPCSSYIPSGTGYSDFDSAYKTCSVVGAVPGSDIVSGDLFLKLS |
| | YGYEHSHKWRGFGVVLAYAIFFFGVYLTFTEYNESAKQKGEIIVFPQAVVRKI |
| | KKMSKSTHDLESASASDESYTDKKLVSDDYDESQDSYNDVGLSESEATLHW |
| | RDLCYDVQIKGETRRILNNVDGWVAKNSITALMGSSGAGKTTLMDCLASRVT |
| | MGVITGDILVNGRLRDEGFPRSIGYCQQQDLHLATATVRESLKFSAYLRQPA |
| | SVSKEEKDAYVESVIKILDMQRYADAVVGVMGEGLNVEQRKRLTIGVELAAK |
| | PKLLMFLDEPTSGLDSQTAWSICQLMRKLANHGQAILCTIHQPSAMLIDQFDR |
| | LLFLQKGGKTAYFGDLGEGCKTMIDYFESKGAPKCPPNANPAEWMLDIVQA |
| | RDYHEAWKSSDEYKQVHATLDEMERELPNIVISNADDTSAFAASFPVQLFYV |
| | YKRVVQQYWRTPIYLWAKFFVTGASELFIGFTFFKANHTLQGLKNQMLAIFMF |
| | WVIFNPFLQQYLPIFTQQRDLYEARERPSRTFSWYAFLIGQMLAEIFPNILCAIL |
| | GFFCFYYPIGFASNASYSGQLVEKSGLFFFYSLIFFTWIGSTALMVAAPFEDP |
| | QAGGHLANLMFTMALSFNGVFVGPGKLPGFWKFMYRVSPLTYFVDGALSIG |
| | LSDNKVECSQYEYTDVQLPAGMTCGEYLSPYIEKVGTGYILDKNATSECKMC |
| | QISTTNAFLTTVSSKYSRRWRNLGIFIAYIAFNYVMAMFLYWWARVPKKANRV |
| | SDEKDSSKDKKDEKK- |
| SEQ ID | ATGTCTTTGGGTACTTCCCATGTTCCAGAAGCTTTGGCTGCTGAATTGGAAGCTGTT |
| NO: 993 | AGAAATAGGGATGAATCTTCTTCAACCGCCGAAAAGTATGAAGGTTTGGATTCTCA |
| | TGCTAAGGATTCCGTTAGAGATTTGGCTAGATCTTTGACTCATGAAGGTGCTGCTGC |
| | TGCAGATGGTTCTGAATCTCCAGTTAATCCAGTTTTCTCTGATCCAAATGCTCCAGAT |
| | TATGATGCTAGATTGGACCCAAATTCCGACGAATTTTCTTCTGCTTTTTGGGTCAGA |
| | AACTTGTCCGAATTGGTTAGACAAGATCCAGATCACTACAAGCCATATTCTTTGGGT |
| | TGTTCTTGGAGAAACTTGAGAGCTTATGGTAACGCTACTGATGTTGCTTACCAATCT |
| | ACCGTTGTTAACATCCCAGTTCAATTGGTTGAAACCGTTTACAGAATGGCTAGAAAA |
| | GCTAGACCAGAAGATACCTTCAACATCTTGAAACCTATGGATGGTTTGGTTAAGCCA |
| | AGAGAGTTGTTGGTTGTTTTGGGTAGACCAGGTTCTGGTTGTTCTACTTTGTTGAAA |
| | ACTGTTTCCGCTAACACCCATGGTTTTCATGTTGATCCAGAATCCAGAATTTCCTACG |
| | ATGGTTTGTCTCCAAAAGAAGTTGCCAAACATTACAGAGGTGAAGTTGTTTACAAC |
| | GCCGAATCTGATGTTCATTTCCCACATTTGACTGTTTTCCAGACCTTAAAGACTGTTG |
| | CAAGATTGGCTTGTCCATCCAATAGAATTCATGGTGTTGATAGAGAAGCCTTCTCCA |
| | CTCATATTACTGAAGTTGCTATGGCTACTTACGGTTTGTCACATACAAGAAACACTA |
| | AGGTCGGTAACGAATTGGTAAGAGGTGTTTCTGGTGGTGAAAGAAAGAGAGTTTC |
| | TATTGCCGAAGTTTCCATTTGCGGTTCTAAGTTTCAATGTTGGGATAATGCTACTAG |
| | AGGTTTGGACTCTGCTACTGCTTTGGAATTCATTAGAGCTTTGAGAACCCAAGCTAA |
| | GTTGACTGCTTCTGCTGCTGTTATTGCTATCTACCAATGTTCTCAAGATGCCTACGAT |
| | TTGTTCGATAAGGTTTCTGTTTTACACGAGGGTTACCAAATCTATTTTGGTCCAGCTA |
| | AAGATGCTAAGGCCTACTTTGAAAGAATGGGTTATGTTTGTGCTCCAAGACAAACT |
| | ACTGCTGATTTTGTTACTGCTGTTACCAATCCAGCTGAAAGACAAGTTAGAGCTGGT |
| | GCTAGACCACCAGCTTCAGCTGTTGATATGGAAACATATTGGAAGCAGTCTCCAGA |
| | ATATGCTCAACTGTTACAAGATATCGATGAATACGAAGCCTCTGGTGGTAACTCTAA |
| | AGAACAATTGGCTGCTAATCATGTCGCTAAGCAATCTAGAAGGGCTAGAGCTGCTT |
| | CTCCATATACTGCTTCATTTTGGTTGCAGGTCAAGTACCTGTTGATCAGAAACATGC |
| | AAAGAATCAGGTCCAATATGGGTGTCACTGCTTTTCAAGTTATTGGTAATGGTTCCA |
| | TGGCTTTCATCTTGGGTTCCATGTTTTACAAGATTCTGAAGCACGATAACACCGCTG |
| | GTTTTTATTCTAGAGCCGGTGCTTTGTTTTTCGCCGTTTTGTTTAATGCTTTCTCGTGC |
| | ATGTTGGAAATCTTGGCATTATATGAAGCCAGGCCAATTTCTGAAAAGCACAAGAG |
| | ATACAGCTTGTATCATCCAGCTGCTGATGCTATGGCATCTGTTATTAGTGAAATTCC |
| | AGCCAAGTTGGTTACCTCTGTTGCTTTTAACTTGGCCTTGTACTTCTTGTGTAACTTC |
| | AAAAGAGAAGCTGGCGCTTTCTTCTTCTACTTCTTGATGACTATGGTTGCCACGTTTT |
| | TGATGTCCCATATTTTCAGATGTTTGGGTGCTTCTACTAAGACCTATGCTGAATCTAT |
| | GGTTCCAGCATCTGTTTTGTTGTTGGGTTTAGCTATCTACACCGGTTTTGCTATTCCA |
| | AAGACCAAGATTTTAGGTTGGTCTAAGTGGATCTGGTACATCAATCCATTGTCCTAC |
| | GTTTTCGAATCCTTGATGGTTAACGAATTCCACGGTAGAAGATTTGCTTGCTATGCT |
| | TATATTCCAACTGGTCCAGGTTACTTGGATGTTACTGGTACTGAACATGTTTGTTCTG |
| | CAGTTGGTGCTGTTCCAGGTCAAAATTATGTTGATGGTGAAACCTACATCAACGTTG |
| | CTTATGGTTATTACCATGCTCATAAGTGGCGTGGTTTAGGTATTGGTTTGGCTTACG |
| | CTATCTTTTTCTTGGGTGTTTACTTGGCCGTTGTCGAATTCAATGAATCTGCTAAACA |
| | AAGGGGTGAGATCTTGGTTTTTCCACATTGGGCTATGGCAAGAATGAAGAAGCAAA |
| | GAAGATTGAGAGCTGCTGGTGCTGATCCTGCTGATGAAGAACATGGTGGTGCTTCA |
| | TCTGGTACTACTGAAAAGAAAATGTTGGAAGATTCCGCCGAAGATGATGATGCTTC |
| | CGCTGGTGCTTCTGCCGATGCTGGTTTGTTGTCATCTTCTAATGCTATCTTTCATTGG |
| | AGGAACGTTTGCTTCGATGTTGCCATTAAGAAAGAAACCAGAAGAATCTTGGATCA |
| | CGTTGATGGTTGGGTAAAACCAGGTACTTTAACTGCTTTGATGGGTGCATCTGGTG |
| | CTGGTAAAACTACTTTATTGGATTGCTTGGCCTCTAGAGTTACTACTGGTGTTATTAC |
| | TGGTGACATGTTCATCAACGGTTTCTTGAGAGATGCTTCTTTCGCTAGATCTATTGG |
| | TTACTGTCAACAACAAGACTTGCATTTGGAAACTGCCACTGTTAGAGAATCTTTGAG |
| | ATTCTCTGCTTACTTGAGACAACCAGATCACGTTTCCATTCAAGACAAGAACTCTTAC |
| | GTTGACGATGTCATTAGGATCTTGGAGATGGAAAAGTACGCTGATGCTGTTGTTGG |
| | TGTTGCTGGTGAAGGTTTAAACGTTGAACAAAGAAAAAGGTTGACCATCGGTGTTG |
| | AATTAGCTGCTAAACCTAAGCTGTTGCTGTTTTTGGATGAACCTACTTCCGGTTTAG |
| | ATTCACAAACTGCTTGGTCTATTTGCCAGTTGATGAGAAAGTTGGCAAATCATGGTC |
| | AAGCTATTTTGTGCACCATTCATCAACCATCAGCTTTGTTGATGCAAGAGTTTGATA |
| | GGTTGCTGTTCTTGCAAAGAGGTGGTAGAACTGTTTACTTCGGTGAATTAGGTGCT |
| | GGTTGTCAAACCATGATTGACTACTTCGAATCACATGGTTCACATAAGTGTCCATCA |
| | GGTGCTAATCCAGCAGAATGGATGTTAGAAGTTATCGGTGCTGCTCCTGGTACTCA |
| | TGCTGCTCAAGATTATAATGAAGTTTGGAGGAATTCCGATGAGTACAGAGCTGTTC |
| | AAGAGGAATTGGAATGGATGGAAAGAGAATTGCCTAATAGACCAGCCGTTGATAC |
| | TTCTTCTGAACAGTCTGAATTTTCCACCTCCTTGGTTTACCAATACTCTTTGGTTACTC |
| | ACAGGTTGTTGCAGCAATATTGGAGAACTCCATCTTACTTGTGGTCTAAGGTTGGTT |
| | TGACCATTATCTCCCAGTTGTTTATTGGTTTCACCTTCTTCAAGTCCGACTCTTCTATG |
| | CAAGGTCTGCAAAATCAAATGCTGTCCATTTTTATGTTCGCCGTCATTTTTAACCCAA |
| | CCTTGCAGCAGTACTTGCCAACATTTGTTTCTCAAAGAGACTTGTACGAAGCCAGAG |
| | AAAGACCATCTAGAACTTTTTCATGGGTTGCCTTCATCTTGTCCCAAATTACTGTTGA |
| | AATCCCCTGGAATATTTTCGCTGGTACAATTGGTTTCTTGGTCTACTATTACCCAGTC |
| | GGTTTTTACTCTAACGCTTCTTATGCTGGTCAATTGCACGAAAGAGGTGCTTTATTCT |
| | GGTTATACGCTACCGCTTTTTACGTTTTCACTGGTTCTATGGCCCAATTAGTTGTTGC |
| | CGGTCAAGAAGTCGCTCAAGCAGCAGGTCAAATTGCTTCTTTGTTGTTTACCCTGTC |
| | TTTGTCTTTCTGTGGTGTTATGGTTCAACCTTACAATATGCCAGGTTTCTGGATTTTC |
| | ATGTACAGAGTTAGTCCACTGACCTACTTTATCGATGGTGCATTGTCTACTGGTATT |
| | GCTAATGCTAAAGTTCATTGCGCCGATTACGAAATGGTTAGGTTTACTCCACCACAA |
| | GGTCAAACATGTGGTCAGTATATGCAACCATATATTACTGCTGCTGGCACTGGTTAC |
| | TTGAAAGATTCTGGTGCTACAGATGAATGCCAATTCTGTTCTGTTTCTACTACCAAC |
| | GATTACTTGAAGGCCGTTTCTTCATCTTATTCTCATAGATGGCGTAACTACGGTATTT |
| | TCTTGGCCTTCATTATGTTCAATTTTGCTGCTGCCGTTTTCTTTTACTGGTTGGCTAGA |
| | GTTCCAAAGAAGAGAAATAGAGTTGCTGACGAAAGAAAGTCCGATGCTCAAAAGT |
| | TGAAAGAGAAGTAA |
| SEQ ID | MSLGTSHVPEALAAELEAVRNRDESSSTAEKYEGLDSHAKDSVRDLARSLTHEGAAAAD |
| NO: 994 | GSESPVNPVFSDPNAPDYDARLDPNSDEFSSAFWVRNLSELVRQDPDHYKPYSLGCSW |
| | RNLRAYGNATDVAYQSTVVNIPVQLVETVYRMARKARPEDTFNILKPMDGLVKPRELL |
| | VVLGRPGSGCSTLLKTVSANTHGFHVDPESRISYDGLSPKEVAKHYRGEVVYNAESDVH |
| | FPHLTVFQTLKTVARLACPSNRIHGVDREAFSTHITEVAMATYGLSHTRNTKVGNELVR |
| | GVSGGERKRVSIAEVSICGSKFQCWDNATRGLDSATALEFIRALRTQAKLTASAAVIAIY |
| | QCSQDAYDLFDKVSVLHEGYQIYFGPAKDAKAYFERMGYVCAPRQTTADFVTAVTNPA |
| | ERQVRAGARPPASAVDMETYWKQSPEYAQLLQDIDEYEASGGNSKEQLAANHVAKQ |
| | SRRARAASPYTASFWLQVKYLLIRNMQRIRSNMGVTAFQVIGNGSMAFILGSMFYKILK |
| | HDNTAGFYSRAGALFFAVLFNAFSCMLEILALYEARPISEKHKRYSLYHPAADAMASVIS |
| | EIPAKLVTSVAFNLALYFLCNFKREAGAFFFYFLMTMVATFLMSHIFRCLGASTKTYAES |
| | MVPASVLLLGLAIYTGFAIPKTKILGWSKWIWYINPLSYVFESLMVNEFHGRRFACYAYI |
| | PTGPGYLDVTGTEHVCSAVGAVPGQNYVDGETYINVAYGYYHAHKWRGLGIGLAYAIF |
| | FLGVYLAVVEFNESAKQRGEILVFPHWAMARMKKQRRLRAAGADPADEEHGGASSG |
| | TTEKKMLEDSAEDDDASAGASADAGLLSSSNAIFHWRNVCFDVAIKKETRRILDHVDG |
| | WVKPGTLTALMGASGAGKTTLLDCLASRVTTGVITGDMFINGFLRDASFARSIGYCQQ |
| | QDLHLETATVRESLRFSAYLRQPDHVSIQDKNSYVDDVIRILEMEKYADAVVGVAGEGL |
| | NVEQRKRLTIGVELAAKPKLLLFLDEPTSGLDSQTAWSICQLMRKLANHGQAILCTIHQP |
| | SALLMQEFDRLLFLQRGGRTVYFGELGAGCQTMIDYFESHGSHKCPSGANPAEWMLE |
| | VIGAAPGTHAAQDYNEVWRNSDEYRAVQEELEWMERELPNRPAVDTSSEQSEFSTSL |
| | VYQYSLVTHRLLQQYWRTPSYLWSKVGLTIISQLFIGFTFFKSDSSMQGLQNQMLSIFM |
| | FAVIFNPTLQQYLPTFVSQRDLYEARERPSRTFSWVAFILSQITVEIPWNIFAGTIGFLVYY |
| | YPVGFYSNASYAGQLHERGALFWLYATAFYVFTGSMAQLVVAGQEVAQAAGQIASLLF |
| | TLSLSFCGVMVQPYNMPGFWIFMYRVSPLTYFIDGALSTGIANAKVHCADYEMVRFTP |
| | PQGQTCGQYMQPYITAAGTGYLKDSGATDECQFCSVSTTNDYLKAVSSSYSHRWRNY |
| | GIFLAFIMFNFAAAVFFYWLARVPKKRNRVADERKSDAQKLKEK |
| SEQ ID | ATGGGTTTGCCAGAAGAACCACAAGATAACTCTTCAGGTTCTTCAGATGT |
| NO: 995 | TCCAGTTTACAATGGTTTCGATGTTAAGGCCCAAGAAAAGGTTAGACAATT |
| | GGCTAGAACTTTGACCGAACAATCTGCTTCTGGTTCTATTGGTGCTGGTG |
| | GTCATACTGCTGATGATGGTGCTGTTGTTGAAAGAGGTGACGCTCAATCT |
| | TTGTTCTCTGTTAGATCTAGAGCTGGTGTTAACCCAGTTTTTGATGGTGAA |
| | TCTGATGCTCAATACGATGCTAGATTGGATCCTAAGTCTGACGAATTTGTT |
| | TCTGCCCAATGGATCAAGAACATGTCCAGATTGTCTCATGAAGATCCAGA |
| | TTACTTCAAGCCATACTCTTTGGATTGCTGTTGGAGAGATTTGTCTGCTTC |
| | AGGTGCTTCTGCTGATGTTGCTTATCAATCTACTTTTGTTAACGCCCCAAA |
| | GAAGTATTTGGGTCAAGCTTATAGAGCTATTACCCCAGCTAAAGAAGTCA |
| | ACAGATTCGAAATTTTGAAGCCAATGGACGGTTTGTTGAACCCAGGTGAA |
| | TTATTGGTTGTTTTGGGTAGACCAGGTTCTGGTTGTACTACTTTGTTGAAG |
| | TCCATTTCCTCTAACACCCATGGTTTCAAGATCTCCAAAGAATCCCATGTT |
| | TCCTACAAGGGTATTTCCCCAAGAGATATTAACAAGCACTTCAGAGGTGA |
| | AGTTGTTTACAACGCTGAAGCCGATATTCATTTGCCACATTTGACTGTTTA |
| | CCAGACCTTGTTGACTGTTGCCAGATTGAAAACTCCAGCTAATAGAGTTA |
| | AGGGTGTTTCTAGAGAAGCTTGGGCTCATCATATGACTGAAGTTGCTATG |
| | GCTACTTATGGTTTGTCTCATACCAGAAACACTAAGGTTGGTAACGATATT |
| | GTCAGAGGTGTTTCAGGTGGTGAAAGAAAGAGAGTTTCTATTGCTGAAGT |
| | CGCTATTTGCGGTTCTAAGTTTCAATGTTGGGATAACGCTACTAGAGGTTT |
| | GGATTCTGCTACTGCTTTGGAATTTGTTAGAGCCTTGAAAACCCAAGCTG |
| | ATATTGCTAATACTGCTGCTGCTGTTGCAATCTACCAATGTTCTCAAGATG |
| | CTTACGATTTGTTCGATAAGGTTTGCGTTTTGGATGGTGGTTACCAAATCT |
| | ATTTTGGTCCAGCTAAGGATGCTAAGAGGTACTTTCAAGAAATGGGTTAC |
| | GTTTGCCCAGATAGACAAACTACAGCTGATTTCTTGACTGCTGTTACTTCT |
| | CCAGCTGAAAGAATCGTCAATGAAGAGTTCATTAAGAAGGGTATCAGAGT |
| | CCCACAAACTCCAAAAGAAATGTCCGATTATTGGAGGAACTCTTCCGAGT |
| | ACAGAAAGTTGATTGCTCAAATTGATGCCAAGATGGCCGAAAACGATGAA |
| | GAAGAAAGAGTCAGGATCAAAGAATCTCATATCGCCTCTCAATCTAAGAG |
| | AGCTAGAGCTTCTTCTCCTTACATGGTTTCTTATATGATGCAGGTCAAGTA |
| | CCTGCTGATTAGAAACGTTTGGAGAATCAGAAACTCCTCCGCTATTGCTTT |
| | GTTCCAAGTTATTGGTAACTCCGTTATGGCCTTGATTTTGGGTTCTATGTT |
| | CTACCAAGTTATGAAGGGTGATTCTACTGCTACCTTCTACTTTAGAGGTTC |
| | TGCTATGTTCTTTGGCTTGTTGTTTAACGCCTTCTCATCCTTGTTGGAAAT |
| | CTTCTCATTATACGAAGCCAGACCAATTACCGAAAAGCACAGAACTTATTC |
| | CTTGTACCATCCTTCAGCTGATGCTTTTGCTTCTGTTTTGTCTGAAATCCC |
| | CACTAAGTTGATGACCGCTGTTTGTTTCAACATCATCTTCTACTTCATGGT |
| | CAACTTCAGAAGAAACGCTGGTAGATTCTTTTTCTACTTCCTGATTAACAT |
| | CGTTGCCACCTTCACTATGTCTCATTTGTTTAGATGTGTCGGCTCTTTGAC |
| | TAAGACTTTATCTGCTGCTATGGTTCCAGCTGCAATGTTGTTGTTGGCTTT |
| | GGCTATTTTTACTGGTTTCGCTGTTCCAGAGACTAAGATGTTAGGTTGGTC |
| | TAAGTGGATTTGGTACATCAACCCATTGTCCTACTTGTTCGAATCCTTGAT |
| | GGTTAACGAATTCCACGGTAGAGAATTTCCATGTGCTCAATTTGTTCCATC |
| | AGGTCCAGCTTACCAAAACATTACTGGTACTGAAAGAGTTTGTTCCGCTG |
| | TTGGTTCAATTCCAGGTGCATCTTCAGTTTTGGGTGATGACTTTTTGAAAA |
| | CCTCCTACAACTACTACAACAAGCACAAGTGGCGTGGTTTTGGTGTTGGT |
| | ATGGCTTATGCTATTTTCTTTTTCGCCGTGTACTTGTTCTTGGCCGAAGTT |
| | AATCAAGGTGCTAAGCAAAACGGTGAGATTTTGGTTTTTCCATTGTCCGTT |
| | GTCAGAAGGTTGAAAAAGCAAAAGAAGTTGTCTCAGCCAAACTCCGGTGG |
| | TGATATTGAAAAAGGTTCAGGTATTGCTTCTACCGTTACCGATAGAAAGTT |
| | GCTGCAAGATTCTTCCGAATCTTTGACCTCTAGTGAAGATGACGTTGGTTT |
| | GTCAAAGTCTGATGCCATTTTTCATTGGAGGGACTTGTGTTACGAAGTCC |
| | AGATCAAGAAAGAAACCAGAAGGATCTTGAACAACGTTGATGGTTGGGTT |
| | AAGCCAGGTACTTTGACTGCTTTGATGGGTGCATCAGGTGCTGGTAAAAC |
| | TACTTTATTAGATTGCTTGGCAGAAAGGACCACTATGGGTGTCATTACAG |
| | GTAACATTTTCGTCAACGGTAACTTGAGGGATGAATCTTTCCCAAGATCTA |
| | TTGGTTACTGTCAACAACAAGACTTGCATTTGACTACCGCTACTGTTAGAG |
| | AAAGCTTGAAGTTTTCTGCCTACTTGAGACAACCAGCTTCTGTTCCAACTG |
| | AAGAAAAAGATAGATACGTCGAAGAAGTGATCAAGGTCTTGGAAATGGAA |
| | ACTTATGCTGATGCAGTTGTTGGTGTTGCTGGTGAAGGTTTGAATGTTGA |
| | ACAAAGAAAGCGTTTGACCATCGGTGTTGAATTGGTTGCTAAACCAGCTT |
| | TGCTTGTTTTCTTGGATGAACCTACATCTGGTTTGGATAGTCAAACTGCTT |
| | GGTCTATTTGCCAGCTGATGAAGAAATTGGCTAACCATGGTCAAGCTATT |
| | TTGTGCACTATTCATCAACCATCCGCTATGTTGATGCAAGAATTTGATAGG |
| | CTGTTGTTCTTGAAAAGAGGCGGCGAAACTGTTTACTTTGGTGAATTAGG |
| | TGATGGTTGCCATACCATGATTGACTACTTTGAAAGAAACGGTGCTCATAA |
| | GTGTCCACCAGATGCTAATCCAGCAGAATGGATGTTGGAAGTTGTAGGTG |
| | CTGCTCCAGGTTCTCATGCTAATCAAGATTATCATCAAGTCTGGCGTTCAT |
| | CCGATGAGTACAAAGCTGTTAGACAAGAATTGGACCAAATGGAAAGAGAA |
| | TTGCCAAACAAGACTACCTCTTTGATGGACGATTCCGAGAAACATAAGTC |
| | TTATGCTACCCCACTGTTCTATCAAATCAAGTTGGTTTGCTTGAGGTTGTT |
| | CCAACAGTATTATAGATCTCCAGGTTACTTGTGGGCTAAGTTCGCTTTGAC |
| | CATTTTCGATAACTTGTTCATCGGTTTCACTTTCTTCAAGGCCGATAGATC |
| | ATTGCAAGGTCTGCAAAATCAAATGCTGTCCATTTTCATGTTCACCGTCCT |
| | GTTTAACAACTTGTTGCAGCAATACTTGCCTTTGTTCGTTCAACAGAGAAA |
| | CTTGTACGAAGCTAGGGAAAGATCTTCCAGAGTTTTTAGTTGGAAGGCTT |
| | TCATTGTCTCCCAAATCGTTGTAGAAGTCCCATGGAATATTTTGGCTGGTA |
| | CTTTGGCTTACCTGATCTATTACTATGCCGTTGGTTTTTACTCTAATGCTTC |
| | AGCTGCTGGTCAATTGCATGAGAGAGGTGCTTTGTTTTGGTTGTTCTCTAT |
| | CGGTTTCTACGTCTACATTGGTTCTATGGGCGTTTTGTGCATTTCCTTTAT |
| | GGAATTGGCTGAAACTGCTGCTAATTTGGCTTCTTTGTTGTTCACCATGTG |
| | TTTGTCTTTCTGCGGTGTTATGGTTACTAAGGATGTCATTCCAAGGTTTTG |
| | GATCTTTATGTACAGATGCAACCCCTTGACTTACTTGATTGAAGCTTTCTT |
| | GGCATTGGGTATTGCTAACGTTAATGTCACCTGTTCCGATTACGAATTGAT |
| | TAAGTTTACTCCAGTGCCAGGTCAAACTTGTGGTGAGTATATGGCTCCAT |
| | ATATTCAAAAAGCTGGTACTGGTTACTTGCAAGACCCAAATGCTACTGATG |
| | TTTGTCAGTTCTGTCAATTCTCCTACACCAACGATTTCTTGGCTACATTGG |
| | GTTGTAAATACTCTCATAGATGGCGTAACTACGGTATCTTCATCTCCTTTA |
| | TCGTTTTCAACTATGTTGCCGCCGTTTTCTTCTACTGGTTGGCTAGAGAAC |
| | CTAAATCTAAGGGTAAGATCTGCAAAAAGAAAGAAGAGAAGTCCAAGAAG |
| | TAA |
| SEQ ID | MGLPEEPQDNSSGSSDVPVYNGFDVKAQEKVRQLARTLTEQSASGSIGAGG |
| NO: 996 | HTADDGAVVERGDAQSLFSVRSRAGVNPVFDGESDAQYDARLDPKSDEFV |
| | SAQWIKNMSRLSHEDPDYFKPYSLDCCWRDLSASGASADVAYQSTFVNAPK |
| | KYLGQAYRAITPAKEVNRFEILKPMDGLLNPGELLVVLGRPGSGCTTLLKSIS |
| | SNTHGFKISKESHVSYKGISPRDINKHFRGEVVYNAEADIHLPHLTVYQTLLTV |
| | ARLKTPANRVKGVSREAWAHHMTEVAMATYGLSHTRNTKVGNDIVRGVSG |
| | GERKRVSIAEVAICGSKFQCWDNATRGLDSATALEFVRALKTQADIANTAAA |
| | VAIYQCSQDAYDLFDKVCVLDGGYQIYFGPAKDAKRYFQEMGYVCPDRQTT |
| | ADFLTAVTSPAERIVNEEFIKKGIRVPQTPKEMSDYWRNSSEYRKLIAQIDAK |
| | MAENDEEERVRIKESHIASQSKRARASSPYMVSYMMQVKYLLIRNVWRIRNS |
| | SAIALFQVIGNSVMALILGSMFYQVMKGDSTATFYFRGSAMFFGLLFNAFSSL |
| | LEIFSLYEARPITEKHRTYSLYHPSADAFASVLSEIPTKLMTAVCFNIIFYFMVN |
| | FRRNAGRFFFYFLINIVATFTMSHLFRCVGSLTKTLSAAMVPAAMLLLALAIFT |
| | GFAVPETKMLGWSKWIWYINPLSYLFESLMVNEFHGREFPCAQFVPSGPAY |
| | QNITGTERVCSAVGSIPGASSVLGDDFLKTSYNYYNKHKWRGFGVGMAYAIF |
| | FFAVYLFLAEVNQGAKQNGEILVFPLSVVRRLKKQKKLSQPNSGGDIEKGSGI |
| | ASTVTDRKLLQDSSESLTSSEDDVGLSKSDAIFHWRDLCYEVQIKKETRRILN |
| | NVDGWVKPGTLTALMGASGAGKTTLLDCLAERTTMGVITGNIFVNGNLRDES |
| | FPRSIGYCQQQDLHLTTATVRESLKFSAYLRQPASVPTEEKDRYVEEVIKVLE |
| | METYADAVVGVAGEGLNVEQRKRLTIGVELVAKPALLVFLDEPTSGLDSQTA |
| | WSICQLMKKLANHGQAILCTIHQPSAMLMQEFDRLLFLKRGGETVYFGELGD |
| | GCHTMIDYFERNGAHKCPPDANPAEWMLEVVGAAPGSHANQDYHQVWRS |
| | SDEYKAVRQELDQMERELPNKTTSLMDDSEKHKSYATPLFYQIKLVCLRLFQ |
| | QYYRSPGYLWAKFALTIFDNLFIGFTFFKADRSLQGLQNQMLSIFMFTVLFNN |
| | LLQQYLPLFVQQRNLYEARERSSRVFSWKAFIVSQIVVEVPWNILAGTLAYLIY |
| | YYAVGFYSNASAAGQLHERGALFWLFSIGFYVYIGSMGVLCISFMELAETAA |
| | NLASLLFTMCLSFCGVMVTKDVIPRFWIFMYRCNPLTYLIEAFLALGIANVNVT |
| | CSDYELIKFTPVPGQTCGEYMAPYIQKAGTGYLQDPNATDVCQFCQFSYTN |
| | DFLATLGCKYSHRWRNYGIFISFIVFNYVAAVFFYWLAREPKSKGKICKKKEE |
| | KSKK- |
| SEQ ID | ATGAACAGCTCTACCCACTACGATGATGTCTCTCAAAAATCTTCTACCGTT |
| NO: 997 | AACGATTCCGCCGATGATAACAAAATTCCAGAATACCATGGTTTCGACAA |
| | AGAGGTTTCTTCTGAAGTTCAAAATTTGGCCAGAATGATGTCCAGACCTTC |
| | TATTGATAAGGCTTCTTCATTGGCTAGAACCCTGTCTAATATGTCTCAAGT |
| | TCCAGGTTTGAATCCAATGGCTAAAGATGAAGGTGAATTGGACCCAAGAT |
| | TGGATCCAGATTCTGATTCTTTCGATTCTAAGTTCTGGGTCAAGAACTTGA |
| | GGAAGATGCATAATTCTGATCCAGCTTACTACAAGCCAGCTTCTTTGGGT |
| | GTTGCTTACAAAGATTTGAGAGCTTACGGTATTGCTACCGATGCTGATTAT |
| | CAAGCTAATGTTGGTAACGTTGTCTACAAGACCATTTCTCAAACCATCAAG |
| | GGTTTCTTCGATAAGAACAACGATGATGCCAAGTTCGATATCTTGAAACCT |
| | ATGGACGGTTTGATTAGACCAGGTGAAGTTACTGTTGTTTTGGGTAGACC |
| | TGGTGCTGGTTGTTCTACATTCTTGAAAACCATTTCTTCTAACACCCACGG |
| | TTTCACTGTTGCTAAAGATTCTGTTTTGTCCTACGACGGTTTAAAGCCAAA |
| | CGATATCATTAAGCACTTCAGAGGTGATGTTGTTTACTGTGCTGAAACCG |
| | AATCTCATTTCCCACAATTGACTGTTGGTCAAACTTTGGATTTTGCTGCTA |
| | AGTTGAGAACCCCTCAAAACAGACCAGAAGGTGTTTCTAGAGAAGAATAT |
| | GCTGCTCATATGACCAAGGTTATTATGGCTACTTATGGTTTGTCCCATACC |
| | AGAAATACCAAGGTTGGTAATGATTTCATCAGAGGTGTTTCAGGTGGTGA |
| | AAGAAAGAGAGTTTCTATTGCTGAAGTTGCTTTGTCCTTCGCTTCTTTACA |
| | ATGTTGGGATAACTCTACCAGAGGTTTGGATTCTGCTACTGCTTTGGAATT |
| | CATTAAGGCTTTGAAAACCTCTGCCACTGTTTTGAATGCTACTCCAATGAT |
| | TGCTATCTACCAATGCTCTCAAGATGCCTACGATTTGTTCGATAAGGTCAT |
| | CTTGTTGTACGAAGGTTACCAAATTTTCTTCGGTGATTGCAAGCAAGCCAA |
| | GTTGTACTTTTTGGAAATGGGTTACGATTGCCCACAAAGACAAACTACTG |
| | CTGATTTCTTGACCTCTTTGACTAACCCATCTGAAAGAGTTGTAAGACCAG |
| | GTTACGAAAACAAGGTTCCAAGAACACCAGAAGAATTCTACACTTACTGG |
| | CAAAACTCTCCAGAAAGAAAAGCTTTGTTGGGTGAAATCGATGACTACTT |
| | GAACAAGACCGATAACGAAGAAAGATTGCAACAATTCAAGGATGCCCATA |
| | ACACCAAGCAATCTAATCACTTAAGACCAGCTTCTCCATACACTGTCTCTT |
| | ACGGTTTACAAGTCAAGTACATCATGGGCAGAAACATTATGAGAACTAAG |
| | GGTGATCCATCCATCACCTTGTTCTCTATTTTTGGTAACACCGTCATGGGC |
| | TTGATCATCTCTTCTATTTTCTACAATTTGGACGACACCACCGGTTCTTTTT |
| | ACTATAGAACAGTTGCTATGTTTTTCGCCGTTTTGTTCAACGCTTTCTCCT |
| | CCTTGTTGGAAATTTTTGCCTTGTATGAAGCCAGACCAATCGTTGAAAAAC |
| | ACAAAACTTATGCCCTGTACCATCCATCTGCTGATGCTTTTGCTTCTATTA |
| | TTACTGAGTTGCCACCAAAGCTGCTGGTTTCTATTTCTTTTAACTTGGTGC |
| | TGTACTTCATGGTCAACTTTAGACGTAATGCTGGCAACTTCTTCTTCTACT |
| | TGTTGGTTAATTTCACCGCCACCTTGTCTATGTCTCATTTGTTTAGAACTAT |
| | TGGCTCCGCTACCAAGTCTTTGTCTCAAGCTATGACTCCAGCTTCAGTTTT |
| | GTTGTTGGCTTTGACTATTTTCACCGGTTTCGTTATTCCAACTCCAGAAAT |
| | GTTAGGTTGGTGTAGATGGATCAATTACTTGGATCCTATTGGTTACGCTTT |
| | CGAAGCTTTGATTGCTAACGAATTCCATGGTAGAGATTTCGACTGTTCTCA |
| | ATTTGTTCCATCTGGTCCAGGTTATCCAACTTCTGGTAACTCTATTATCTG |
| | CTCCGTTGTTGGTTCACAACCAGGTTCTGATATAGTTAACGGTGATGACT |
| | ACATTAGGGGTTCTTACGAGTACTACTTTTCTCATAGATGGCGTAATTGGG |
| | GTATTGTCGTTGGTTTCGTTGTTTTCTTCTTGTTCGTTCACATTATCATCTG |
| | CGAGTACAACAAAGGTGCTATGCAAAAGGGTGAGATCCTGTTGTTTCAAA |
| | GATCTGCTTTGAAGAAGAACAAGAGACAACGTAAGGATATCGAGTCCGGT |
| | AACATTGAAAAAGTTGGTCCAGAATTCAACAACGAGAAAACCCCAGATAA |
| | CGAAATCGATAACAAGTTGCCATCCTCCGGTGATATTTTTCATTGGAGAG |
| | AATTGACCTACCAGGTCAAGATTAAGTCCGAAGATAGAGTCATCTTGAAC |
| | TCTGTTGATGGTTGGGTTAAGCCTGGTCAAGTTACTGCTTTAATGGGTGC |
| | TTCAGGTGCTGGTAAAACTACTTTGTTGAATGCCTTGTCTGACAGATTGAC |
| | CTCTGGTGTTATTACTTCAGGTGTCAGAATGGTTAATGGTCACGAATTGG |
| | ATGCTTCGTTCCAAAGATCAATCGGTTACGTTCAACAACAAGACTTGCACT |
| | TGCAAACCTCTACTGTTAGAGAAGCCTTGACTTTTTCTGCCTATTTGAGAC |
| | AACCTAAGTCTGTCCCAAAATCCGAAAAGGATTCTTACGTTGATTACATCA |
| | TCAGGTTGTTGGAGATGGAAAAGTACTCTGATGCTGTTGTTGGTGTTAGT |
| | GGTGAAGGTTTAAATGTCGAACAGAGAAAAAGATTGACCATCGGCGTTGA |
| | ATTGGTTGCTAAACCTAAGTTGTTGGTCTTTTTGGATGAACCTACATCTGG |
| | TTTGGATAGTCAAACTGCTTGGTCTATCTGCAAGTTGATTAGAAAGTTGGC |
| | TGATCACGGTCAAGCTATTTTGTGTACTATTCATCAACCATCCGCCATCCT |
| | GTTGAAAGAATTTGATAGACTGTTGTTCTTGCAGAGAGGTGGTAAGACTG |
| | TTTACTTTGGTGATTTGGGTGATAACTGCCAAACCTTGATTGACTACTTTG |
| | AAAAGTACGGTGCTCCAAAATGTCCACCAGATGCTAATCCAGCTGAATGG |
| | ATGTTGGAAGTTATTGGTGCTGCTCCAGGTTCTCATGCTTCACAAGATTAT |
| | TATGATGTCTGGATGAACTCCACCGAGTACAGAGAAGTTAAGGGTGAATT |
| | AGATGTCATGGAACAAGAGTTGGTGAAAAAGCCAAAAGACGATTCTCCAG |
| | AATCTATGAAGACTTTCGCTGTTCCAATGTGGCAACAGTACATTAACGTTA |
| | CTCATAGAGTCTTGCAGCAGTATTGGAGAACTCCATCTTATACCTACTCCA |
| | AGGTTTTGATGTCCATCTTCTCGTCTTTGTTTAACGGTTTCGCTTTCTTCAA |
| | GGCTAACAACACTATGCAAGGCTTGCAAAATCAAATGTTCTCCGTGTTCAT |
| | GTTCTTCGTCATTTTCAACACTTTGACCCAGCAATACTTGCCCAACTATGT |
| | TTCTCAAAGAGACTTATACGAAGCCAGAGAAAGACCATCTAAAACCTTTTC |
| | TTGGGTTGCTTTCATTACCGCTCAAATTACAGCTGAAATCCCATGGCAAAT |
| | ATTGACTGGTACTTTGGCTTTCTTCTGTTGGTATTATCCAATTGGCTTGTA |
| | CGGTAATGCTGAAGCTACTGATACTGTTTCACAAAGGGGTGCTTTGATGT |
| | GGATTATCATCGTCTTGTTTTTCATCTACTGCTCCACTATGGCTCAATTGT |
| | GCATTTCCTTTATGGAAGTTGCTGATAACGCTGCTAATTTGGCCTCTTTGT |
| | TGTTCACTATGTGTTTGACTTTCTGCGGTATTTTGGCTTCTCCAGATGCAA |
| | TGCCAGGTTTCTGGATTTTTATGTACAGATGCAACCCCTTCTCCTACTTGG |
| | TTTCTGCTATTTTATCTGTTGCCTTGCAGGATTCCTCTGTTACTTGTTCTGA |
| | CAAAGAACTGTTGAGATTCGAACCTGAAGGTGGTCAAACATGTGGTGAGT |
| | ATATGCAATCTTACATCCAAAATGCTGGTGGCTACTTGATCTCTAATGATA |
| | CAACTGGTTCTTGCGAATACTGCACTATCTCTTCCACTAACGTTTTCTTGG |
| | AATCCGTTTCTGTTGACAAGACCAAGAGATCTAGAGATATCGGTATTTTCT |
| | TCTGCTTCATCGTCATCAACATGATTGGCACCGTTTTCTTTTATTGGTTGG |
| | CTAGAGTTCCTAAGAGGTCCAGACAAAAGTAA |
| SEQ ID | MNSSTHYDDVSQKSSTVNDSADDNKIPEYHGFDKEVSSEVQNLARMMSRP |
| NO: 998 | SIDKASSLARTLSNMSQVPGLNPMAKDEGELDPRLDPDSDSFDSKFWVKNL |
| | RKMHNSDPAYYKPASLGVAYKDLRAYGIATDADYQANVGNVVYKTISQTIKG |
| | FFDKNNDDAKFDILKPMDGLIRPGEVTVVLGRPGAGCSTFLKTISSNTHGFTV |
| | AKDSVLSYDGLKPNDIIKHFRGDVVYCAETESHFPQLTVGQTLDFAAKLRTPQ |
| | NRPEGVSREEYAAHMTKVIMATYGLSHTRNTKVGNDFIRGVSGGERKRVSIA |
| | EVALSFASLQCWDNSTRGLDSATALEFIKALKTSATVLNATPMIAIYQCSQDA |
| | YDLFDKVILLYEGYQIFFGDCKQAKLYFLEMGYDCPQRQTTADFLTSLTNPSE |
| | RVVRPGYENKVPRTPEEFYTYWQNSPERKALLGEIDDYLNKTDNEERLQQF |
| | KDAHNTKQSNHLRPASPYTVSYGLQVKYIMGRNIMRTKGDPSITLFSIFGNTV |
| | MGLIISSIFYNLDDTTGSFYYRTVAMFFAVLFNAFSSLLEIFALYEARPIVEKHK |
| | TYALYHPSADAFASIITELPPKLLVSISFNLVLYFMVNFRRNAGNFFFYLLVNFT |
| | ATLSMSHLFRTIGSATKSLSQAMTPASVLLLALTIFTGFVIPTPEMLGWCRWIN |
| | YLDPIGYAFEALIANEFHGRDFDCSQFVPSGPGYPTSGNSIICSVVGSQPGSD |
| | IVNGDDYIRGSYEYYFSHRWRNWGIVVGFVVFFLFVHIIICEYNKGAMQKGEIL |
| | LFQRSALKKNKRQRKDIESGNIEKVGPEFNNEKTPDNEIDNKLPSSGDIFHWR |
| | ELTYQVKIKSEDRVILNSVDGWVKPGQVTALMGASGAGKTTLLNALSDRLTS |
| | GVITSGVRMVNGHELDASFQRSIGYVQQQDLHLQTSTVREALTFSAYLRQPK |
| | SVPKSEKDSYVDYIIRLLEMEKYSDAVVGVSGEGLNVEQRKRLTIGVELVAKP |
| | KLLVFLDEPTSGLDSQTAWSICKLIRKLADHGQAILCTIHQPSAILLKEFDRLLF |
| | LQRGGKTVYFGDLGDNCQTLIDYFEKYGAPKCPPDANPAEWMLEVIGAAPG |
| | SHASQDYYDVWMNSTEYREVKGELDVMEQELVKKPKDDSPESMKTFAVPM |
| | WQQYINVTHRVLQQYWRTPSYTYSKVLMSIFSSLFNGFAFFKANNTMQGLQ |
| | NQMFSVFMFFVIFNTLTQQYLPNYVSQRDLYEARERPSKTFSWVAFITAQITA |
| | EIPWQILTGTLAFFCWYYPIGLYGNAEATDTVSQRGALMWIIIVLFFIYCSTMA |
| | QLCISFMEVADNAANLASLLFTMCLTFCGILASPDAMPGFWIFMYRCNPFSYL |
| | VSAILSVALQDSSVTCSDKELLRFEPEGGQTCGEYMQSYIQNAGGYLISNDT |
| | TGSCEYCTISSTNVFLESVSVDKTKRSRDIGIFFCFIVINMIGTVFFYWLARVPK |
| | RSRQK- |
| SEQ ID | ATGGAAACCGACTCCTCATCTTCTAAACCAGATGGTTATCATGGTTTGGA |
| NO: 999 | CTCTAAGACTGAAGAACACATTAAGGCTTTGGCTAGAACTTTGTCTAGAAC |
| | CTCTTTGAACAGACAGATTTCCTTGCAAGCTTCTGGTAATGGTGCTTCTAA |
| | ATTCGATGAAGTCAAGTCCATCTTCTCCTCTTCATACTCTGGTGTTAATCC |
| | AGTTTTCTTGGATCCATCTGCTCCAGGTTATGATGCTAGATTAGATCCAAA |
| | CTCCGAACATTTTTCTTCAGCTGCTTGGGTTAAGAACATGGTTGCTTTTTC |
| | TATGCAAGACCCCGATTACTACAAGCACTATACAATTGGTTGTTGCTGGA |
| | AGGATTTGAGAGCTTTTGGTGATTCTAACGATGTCTCTTACCAATCTACTG |
| | TTACTACTTTGCCAGGTAAGTACTTGGGTAAGATCAAGAGACATTTCACC |
| | GCTACAAAAGAAGAGGACGTTTTCGATATTTTGAAGCCAATGGATGGTTT |
| | GGTTAAGCCAGGTGAATTATTGGTTGTATTGGGTAGACCAGGTGCTGGTT |
| | GTTCTACTTTGTTGAAAACTATTTCCGCCAACGTCGAAGGTTACTCTATTG |
| | ATCCAAATTCCACCATCTCTTACAACGGTTTGGATCCTAAGGTTATCAAAA |
| | AGCACTTCAGAGGTGAAGTTGTTTACAACGCTGAAGGTGATATTCATTTC |
| | CCACATTTGACTGTCTACGAAACCTTGAATATCGTTGCTTTGTTGGCTACT |
| | CCATCCAATAGAATCAAAGGTGTGTCTAGAGAAGAATTCGCTAAGCACAT |
| | TACTGAAGTTGCTATGGCTACTTATGGTTTGTCTCATACCAAGAACACAAA |
| | GGTTGGTGGTGATTTGGTTAGAGGTGTTTCTGGTGGTGAAAGAAAGAGA |
| | GTTTCTATTGCCGAAGTTACCATTTGCGGTTCTAAGTTTCAATGTTGGGAT |
| | AACGCTACTAGAGGTTTGGATTCTGCTACTGCTTTGGAATTCATTAGAGC |
| | CTTGAAAACCTCCACCGATATTTCTGGTTCTACTGCTGTTATTGCTATCTA |
| | CCAATGTTCCCAAGATGCCTACGATTTGTTCGATAAGGTTTGCGTTTTGGA |
| | TGAAGGTTACCAAATCTTTTTCGGTTACGCTAAGGATGCCAAAAAGTACTT |
| | CGAAAACATGGGTTATGTTTCCCCAGCTAGACAAACTACTGCTGATTTCTT |
| | GACAGCTGTTACTAATCCAGCTGAAAGAATCGTTAATCAGGACTACGTAA |
| | AAGAAGGCAGATTCATTCCATCTACCGCCAAAGAAATGGAAGAATATTGG |
| | AGAAATTCCCCAGAGTACAAGCAATTGCATGCCGATATTGAAGAAGAGTT |
| | GTCCAAAGATTCCGCCAAGAATTTCCAACAGTTGCAAGAATCTCATGTCG |
| | CCAAACAATCTAAGGGTCAAAGAAAAGGTTCTCCCTACATCGTTAACTACA |
| | GAATGCAAGTGAAGTACCTGACCATCAGAAACATCATGAGAATCAAGAGA |
| | TCCTTCTCTGTTACCTTGGGTTCTATTTTCGGTAACATCTTCATGTCCTTG |
| | ATCCTGGGTTCCATCTTTTACAAATCTATGAAGCACACGAACACCAACAC |
| | GTTTTTCTATAGAGGTGCTGCTATGTTTACCGCCGTTTTGTTTAATTCCTT |
| | CAGCTCCATGTTGGAGATCTTCACATTATATGAAGCCAGACCAATCACTG |
| | AAAAGCACAAGAGATACAGCTTGTATCATCCTTCTGCTGATGCTTTAGCTT |
| | CCATGATGTCTGAATTGCCAGCTAAAATCGTTACCGCTATTTGCTTCAACC |
| | TGATCTTGTACTTTATGGCCAACTTCAGAAGAGAACCAGGTCCATTCTTTT |
| | TCTACTTCATGATGTCATTCTTGGCCACCTTGGTTATGTCCTCTATTTTCA |
| | GATGTATTGGTGCTGCTGCTAAGACTTTGTCTGAAGCTATGGTTCCATCC |
| | TCCATTTTGTTGTTGGCCATTTCATTATACGTCGGTTTCTCTGTTCCAAAG |
| | AAGTCTTTGTTAGGTTGGTCTAAGTGGATCTGGTACATTAACCCAATCTCG |
| | TACATCTTCGAGAGCTTGATGATCAATGAATTCAACGGTAGAGAATTCCCA |
| | TGCGCTGTTTTTATTCCATCAGGTCCTGGTTACGAAAATGTTTCTGCTACA |
| | GAAAAGGTCTGTAACACCGTTGGTTCAAAACCAGGTTTGCCATACGTTTC |
| | TGGTAAGGATTTTATCGTTCAAGCCTATGGTTATGATCCAGCTCATAGATG |
| | GCGTGGTTTTGGTATTGCTTTGGCTTACTTCATTTTCTTCTCCGCCGTTTA |
| | CTTGTTGTTCTGTGAGTATAACGAATCCGCTGTTCAAAAGGGTGAAATCTT |
| | GGTTTTTCCAAAGGCCGTTTTGAAGAAGGCTAAGAAAGAAGCTTTATCCA |
| | GACCAAAGTCTGATATCGAAACTGGTGAAGATCCAGAAGGTGGTATTACT |
| | GATAGAAAGTTGTTGCAGGATTCCCAAGAAGATTCCAACGAATCTGTTGA |
| | CGAAAAGCAATCCGAAATTGCCTTGGAAAAATCTGGTGCTGTTTTTCATTG |
| | GAGGAACGTTTGTTACGATGTCCAGATCAAGAAAGAAACCCGTAGAATCT |
| | TGTCTAACGTTGATGGTTGGGTGAAACCAGGTACTTTGACTGCTTTGATG |
| | GGTTCTTCTGGTGCAGGTAAAACTACTTTATTGGACTGTTTGGCTTCCAGA |
| | GTTACCATGGGTGTTATTACTGGTGATATGTTCGTTAACGGTCACTTGAGA |
| | GATAACTCATTCCCTAGATCTATTGGTTACTGTCAACAACAAGACTTGCAT |
| | TTGTCTACTGCTACCGTTAGAGAATCCTTGAGATTTTCTGCTTACTTGAGA |
| | CAACCATCCTCCGTTAGTATTGAAGATAAGAACAGATACGTCGAACACGT |
| | CATCAGAATGTTAGGTATGGAAAAGTACGCTGATGCTGTTGTTGGTGTAA |
| | CAGGTGAAGGTTTGAATGTCGAACAAAGAAAGCGTTTGACCATCGGTGTT |
| | GAATTGGCTGCTAAACCTAAGTTGTTGTTGTTTTTGGACGAACCTACCTCT |
| | GGTTTGGATAGTCAAACTGCTTGGTCTGTTTGTCAGCTGATGAGAAAATT |
| | GGCTGATCATGGTCAAGCTATTTTGTGCACTATTCATCAACCATCCGCTTT |
| | GTTGATGCAAGAATTTGATAGGCTGCTGTTCTTGCAAAAAGGTGGTAAGA |
| | CTGTTTACTTCGGTGACTTAGGTCATGGTTGCCAAACTATGATTGATTACT |
| | TTGAAAGAAACGGTGCCCATAAGTGTCCTGAAGGTGCTAATCCTGCAGAA |
| | TGGATGTTGGAAGTTATTGGTGCAGCTCCTGGTTCTTCATCTACAGTTGAT |
| | TACCATGAAGTTTGGCGTAACTCTGAAGAGTACAGAATGGTCCAAAAAGA |
| | ACTAGACTGGATGGAAGTTGAGTTGGCTAAAAAGCCCATGGATACAACCG |
| | AAGAACAAAACGAATTTGCTACCTCTTTGCCCTACCAATTCAAGATAGTTA |
| | CTACCAGGTTGTTCCAACAATATTGGCGTACACCATCTTACATTTGGTCCA |
| | AGGCATTTTTGACCGTTTTCTCCCAAATTTTCATCGGCTTCACTTTCTTCAA |
| | AGCCAAGTTGACATTGCAAGGCTTGCAGAATCAAATGTTCGCTACTTTCA |
| | CTTTCACCATCATTTTCAACCCAGCTTGCCAACAATACTTGCCCTTGTTTG |
| | TTTCTCAAAGGGACTTGTATGAAGCTAGAGAAAGACCATCTAGAACGTTTT |
| | CATGGTTGGCTTTCATCTACTCCCAAATCATCGTTGAAATCCCATTCAACA |
| | TCTTGTTGGGTACTGTTGCTTTCTTCGTTTTCTATTACCCAGTGGGTTTTTA |
| | CAACAACGCTTCTTATGCTGGTCAATTGCACGAAAGAGGTGTCTTGTTTT |
| | GGTTGTTTTGTGTCGAGTTTTACATCTACATCTCTTCCATGGCTCAATTGT |
| | GTATTGCAGGTTTGGAACATGCTGAATCTGCTGGTAACATTTCCTCTATCT |
| | TGTTCACCATGTCTTTGATGTTCTGTGGTGTTTTCGGTGGTCCAGGTGTTT |
| | TGCCTAAATTTTGGAATTTCATGTACCGTGTCTCTCCCTTGACATACTTCA |
| | TTGATGGTTTGTTGTCTACCGGTTTGGCTAATACCAAAGCTGTTTGTGCTG |
| | ATTACGAATACGTTCACTTCGATCCAAAATCCGGTCAAACTTGTGGTAAAT |
| | ACTTGGCCAACTACATCAAAGCCTTTGGTGGTTACTTGGCTAATCCAGAT |
| | GCTACTAATGATTGCTCCTACTGCAAGATCTCTGAATCTAACACTTTCCTG |
| | AAGTCCTTCAAGTCCTCTTATCATAAGCGTTGGAGAAACTTCGGTATCTTC |
| | TTAGTGTTTATCGTTTTTGATTGGGCTGCCTGCATGTTCTTGTATTGGTTA |
| | GCTAGAGTTCCTAAGAAGAAAAACAGAGTCTCCAATGAGAGAAACCCAGA |
| | CAAGTTGAACAATCAAGAGGACAAGAACAAAGAGAAGGTCTAA |
| SEQ ID NO: | METDSSSSKPDGYHGLDSKTEEHIKALARTLSRTSLNRQISLQASGNGASKF |
| 1000 | DEVKSIFSSSYSGVNPVFLDPSAPGYDARLDPNSEHFSSAAWVKNMVAFSM |
| | QDPDYYKHYTIGCCWKDLRAFGDSNDVSYQSTVTTLPGKYLGKIKRHFTATK |
| | EEDVFDILKPMDGLVKPGELLVVLGRPGAGCSTLLKTISANVEGYSIDPNSTIS |
| | YNGLDPKVIKKHFRGEVVYNAEGDIHFPHLTVYETLNIVALLATPSNRIKGVSR |
| | EEFAKHITEVAMATYGLSHTKNTKVGGDLVRGVSGGERKRVSIAEVTICGSK |
| | FQCWDNATRGLDSATALEFIRALKTSTDISGSTAVIAIYQCSQDAYDLFDKVC |
| | VLDEGYQIFFGYAKDAKKYFENMGYVSPARQTTADFLTAVTNPAERIVNQDY |
| | VKEGRFIPSTAKEMEEYWRNSPEYKQLHADIEEELSKDSAKNFQQLQESHVA |
| | KQSKGQRKGSPYIVNYRMQVKYLTIRNIMRIKRSFSVTLGSIFGNIFMSLILGSI |
| | FYKSMKHTNTNTFFYRGAAMFTAVLFNSFSSMLEIFTLYEARPITEKHKRYSL |
| | YHPSADALASMMSELPAKIVTAICFNLILYFMANFRREPGPFFFYFMMSFLAT |
| | LVMSSIFRCIGAAAKTLSEAMVPSSILLLAISLYVGFSVPKKSLLGWSKWIWYI |
| | NPISYIFESLMINEFNGREFPCAVFIPSGPGYENVSATEKVCNTVGSKPGLPY |
| | VSGKDFIVQAYGYDPAHRWRGFGIALAYFIFFSAVYLLFCEYNESAVQKGEIL |
| | VFPKAVLKKAKKEALSRPKSDIETGEDPEGGITDRKLLQDSQEDSNESVDEK |
| | QSEIALEKSGAVFHWRNVCYDVQIKKETRRILSNVDGWVKPGTLTALMGSSG |
| | AGKTTLLDCLASRVTMGVITGDMFVNGHLRDNSFPRSIGYCQQQDLHLSTAT |
| | VRESLRFSAYLRQPSSVSIEDKNRYVEHVIRMLGMEKYADAVVGVTGEGLNV |
| | EQRKRLTIGVELAAKPKLLLFLDEPTSGLDSQTAWSVCQLMRKLADHGQAIL |
| | CTIHQPSALLMQEFDRLLFLQKGGKTVYFGDLGHGCQTMIDYFERNGAHKC |
| | PEGANPAEWMLEVIGAAPGSSSTVDYHEVWRNSEEYRMVQKELDWMEVEL |
| | AKKPMDTTEEQNEFATSLPYQFKIVTTRLFQQYWRTPSYIWSKAFLTVFSQIFI |
| | GFTFFKAKLTLQGLQNQMFATFTFTIIFNPACQQYLPLFVSQRDLYEARERPS |
| | RTFSWLAFIYSQIIVEIPFNILLGTVAFFVFYYPVGFYNNASYAGQLHERGVLF |
| | WLFCVEFYIYISSMAQLCIAGLEHAESAGNISSILFTMSLMFCGVFGGPGVLPK |
| | FWNFMYRVSPLTYFIDGLLSTGLANTKAVCADYEYVHFDPKSGQTCGKYLAN |
| | YIKAFGGYLANPDATNDCSYCKISESNTFLKSFKSSYHKRWRNFGIFLVFIVFD |
| | WAACMFLYWLARVPKKKNRVSNERNPDKLNNQEDKNKEKV- |
| SEQ ID NO: | ATGGAAGCCTCTGAATACGGTGGTTTTGATCATAATGCTGAAAACGAGGT |
| 1001 | TAAGAAGATGGCTAAGGCTTTGGTCAAAGAGAACTCTAACAATAGACCAC |
| | TGGACTCCTCATCTATTTCTAGAGTTACTTCTGGTATTAACCCAGTCGGTT |
| | TACAAGAAACTGATACTGGTTACAACGCTAAGTTGGACCCAAATTCTAAC |
| | GACTTCTCATCTGAAGAATGGACCAGAAATTTGGCCAACATCGTTGAATC |
| | TGAACCAGAGTATTACAAGCCACATTCTGTTGGTTGTTGTTGGAAGAATTT |
| | GTCTGCTGCTGGTGATTCTGCTGATTTGACTTACCAAACTACTTTCGGTAA |
| | CTTGCCAGTCAAGATCTTCAGTTTGTTGTACAGACATATCAGGCCATCTAA |
| | GGGTTCTGATAACTTCCAAATTTTGAAGCCAATGGATGGTTTGGTTGACC |
| | CAGGTGAATTATTGGTTGTTTTAGGTAGACCAGGTTCTGGTTGTACTACTT |
| | TGTTGAAGTCTATTTCCTGTAACACCCACGGTTTTAACGTTGGTAAAGATT |
| | CCGTTATCAAGTACTCTGGTATGGCCTCTAAGGACATTAGAAGAAATTTGA |
| | GAGGTGAAGTTGCTTACAACGCCGAATCTGATACTCATATTCCAAACATC |
| | ACTGTCTACCAAACCTTGGTTACTGTTGCTAGATTGAAAACCCCAAGAAAC |
| | AGAATCAGAGGTATCGATAGAGAATCCTGGGCTAAACATATTGCTGAAGT |
| | TACTATGGCTACCTACGGTTTGTTGCATACCAGAAATACCAAAGTTGGTAA |
| | CGACTTGATCAGAGGTGTTTCTGGTGGTGAAAGAAAGAGAGTTTCTATAG |
| | CTGAAGCTACCATTTGTGGTGCTAAGTTTCAATGTTGGGATAACGCTACTA |
| | GAGGTTTGGATTCTGCTACTGCTTTGGAATTTGTTAGAGCTTTGAGAACC |
| | CAAGCCAAGATTAACAATTCTACTGCTTGTGTTGCTATCTACCAATGCTCT |
| | CAACATGCTTACGATTTGTTCGATAAGGTTTGTGTCTTGTATGGTGGTTAC |
| | CAGATCTATTTTGGTCCAACCTCTAACGCCAAAAAGTACTTCGAATCTATG |
| | GGTTACTACTGCCCATCTAGACAAGCTACAGCTGATTTCTTGACTTCTATT |
| | ACTTCTCCAGCCGAAAGAATCGTCAACAGAGAATTTTTGGAACGTAACAT |
| | CGTCGTTCCACAAACTCCAGACGAAATGTACTTGTACTGGCAAAATTCTC |
| | CAGAAAACCACGAATTGCAAAGGGAAATCGATTCTAGATTGACGCAGGAC |
| | TACAAAGAAAACTTGACAGCTATTAAGGCTTCCCATAATGCCGCTCAATCT |
| | AAGAGAGCTAGAAAATCTTCTCCATACACCGTTTCTTATGCCATGCAAGTT |
| | AAGTACCTGCTGATTAGAAATATGTGGCGTATCATTAACTCTCCAGGTGTT |
| | ACTTTGTTCAGAGTGTTCGGTAACATCATCATGTCTTTGGTTTTGGGCTCT |
| | ATGTTCTACAAGGTTCAAAAGACTACTACCACCAACACCTTTTATTACAGA |
| | GGTGCTGCTATGTTTTACTCCATTTTGGTTAACGCCTTCAGCTCCTTGATT |
| | GAAATTTTCGCTTTGTTCGAAGCTAGACCAGTTACTGAAAAACATAAGACT |
| | TACGCCTTGTATAGACCATCTGCTGATGCTTTTGCTTCCTTCTTGTCTGAT |
| | ATTCCAGGTAAGGTTGTTTCTACCGTTGCCTTCTCTATTATCTACTACTTCT |
| | TGGTCAACTTCAGAAGAGATGCCGGTAGATTTTTCTTCTACCTGTTGATTA |
| | ACTTGGTCACCACTTTCACCATGTCTCACTTGTTTAGATGCGTTGGTTCCA |
| | TTTCTAAGACTTTGACTGAAGCTATGGTTCCAGCTAGTGTTTTGTTGTTGA |
| | CTTTGGCTGTTTACACCGGTTTCTCTATTCCTAGAAGATCTATGCATGAAT |
| | GGTCCAAGTGGATCTCTTACATTGATCCATTGTCCTACTTGTTCGAATCCG |
| | TTATGACTAACGAATTCCATGGTAGAAATTTCCCATGCGCTGCTTACATTC |
| | CAAATGGTCCACAATATCAAAACACCACCGGTACTGAAAGAGTTTGTTCT |
| | GTTGTTGGTTCTAAGGCTGGTCATGATTATGTTTTGGGTGATGACTACTTG |
| | AAGCAGTCTTACGAATACGAAATCAAACATAAGTGGCGTGGTTTTGGTGT |
| | TGGTATGGCTTATGTTGTTTTCTTCTTCTTCGTCTACTTGCTGATCTGTGA |
| | GTATAATGAAGCTGCTAAGCAAAAGGGTGACTTGTTGGTTTTTCCAAGAT |
| | CCATCGTTAAGAAAATGAGAAGGCAGGGTACATTGCAGAAGTCTAATTCA |
| | GAACCTGAAGATGTTGAAAAGAACGCCACCATTATTGCTAACGTTTCTCC |
| | AGATTCCTCCTTGTTATTGGATACTTTGGAATCCCCATCCAAGCAAAAGTC |
| | TGAAATCCCATTGAATCAATCCGACACCATTTTCCATTGGAGAAATGTTTG |
| | CTACGATGTCCACATCAAGAAAGAGTCTAGAAGGATCTTGAACAACGTTG |
| | ATGGTTGGGTTAAGCCAGGTACTTTGACAGCTTTGATGGGTGCTTCTGGT |
| | GCTGGTAAAACAACTTTGTTGGATTGCTTGGCTAAAAGGGTTACCACTGG |
| | TGTTATTACTGGTGAAATCTTCGTCAACGGTAAATTGAGGGATGAATCTTT |
| | CCCAAGATCAATCGGTTATTGTCAACAGCAAGACTTGCATTTGAAAACCG |
| | CTACTGTTAGGGAATCCTTGTTGTTTAGTGCTATGTTGAGACAACCTAAGA |
| | AGGTTCCATTGTCTGAAAAGAAGCAATACGTCGAACAGATCATCGAAGTT |
| | TTGGAAATGGTTCCATACGCTGATGCTATCGTTGGTATTGAAGGTGAAGG |
| | TTTGAACGTGGAACAGAGAAAAAGATTGACCATCGGTGTTGAATTGGTTG |
| | CTAAACCTAAGCTGCTGTTGTTTTTGGATGAACCTACATCTGGTTTGGACT |
| | CTCAAACTGCTTGGTCTATTTGCCAGTTGTTGAGAAAGTTGGCTAACAGA |
| | GGTCAAGCTGTTTTGTGTACTATTCATCAACCATCCGCCTTGTTGATTAGA |
| | GAATTTGATCGTTTGCTGTTCTTGCAGAAAGGTGGTAAGACTGTTTACTTT |
| | GGTGAATTGGGTGACGAATGCAAGACCATGATTGATTACTTCGAAAGAAA |
| | TGGTGCTCATAAGTGTCCACCAGATGCTAATCCAGCTGAATGGATGTTGG |
| | AAGTTGTTGGTGCTGCTCCAGGTACTCATGCTTCTCATGATTACAATGAA |
| | GTTTGGAGGAACTCCGAAGAGTACAAAGAAGTTCAACAAGAATTGGACAG |
| | GATGGAATCTGAATTGAAATGTGTCGGTGAAGAGGACTCTTCCGAAAAAC |
| | ATCAAGCTTTTGCTACCGACATCTTCTCCCAAATATTGATTGTCTCTCACA |
| | GATTCTTCCAGCAGTATTGGAGAACACCACAATATTTGTGGCCAAAGTTC |
| | ATTTTGACTGCCTTCGACGAAATTTTCATCGGCTTTACTTTCTTCAAGGCC |
| | ACCAGATCTTTACAAGGCTTGCAAAATCAAATGCTGTCCACCTTTGTTTTC |
| | TGCGTTGTTTACAATGCTTTGTTGCAGCAATTCTTGCCAGTTTACGTTGAG |
| | CAAAGAAACTTGTACGAAGCAAGAGAAAGACCATCTAGAACCTTCTCTTG |
| | GTTTTCCTTCATCTCCTCACAAATCTTGGTTGAAGTTCCCTGGAACATTAT |
| | TGCAGGTACTATTGCTTTCTTCGTGTACTACTATCCAGTTGGTTTCTACGC |
| | TAATGCCTCTGAAGCTAATCAATTGCACGAAAGAGGTGCATTATACTGGTT |
| | GTTCTGTACCGCTTTCTTTGTTTGGATAGGTTCCATGGGTATTCTGGCCAA |
| | TTCTTTCTTGGAACATGCTGCTGAAGCTGCAAATGTTGCTTTATTGTGTTT |
| | CGCTTTCTCTTTGGCTTTTTGCGGTGTTTTGGTTCCACCAAAGTTATTGCC |
| | AGGTTTCTGGATCTTTATGCACAGAGTTTCTCCACTGACCTACTACATTGA |
| | TTCAGCTTTGTCTTTGGGTATTGCCAACGTTGACGTTAAGTGCTCTGAAAT |
| | CGAATACGTTAAGTTCACTCCACCATCTAACAGAACTTGTGGTCAGTATAT |
| | GCAAGCCTACATTAAGTCTATTGGTACAGGTTATTTGGCTGATCCATCTGC |
| | TACTAATGAATGCAACTTCTGCAGATTGTCTAAGACCAACGATTACCTGAA |
| | GCAGATCTCATCTTCATATTCTCATAGATGGCGTAACTACGGTATCTTCAT |
| | TTGCTTCATCGTTTTCAACTATGTTGCCGCCGTTTTCTTGTATTGGTTGGC |
| | TAGAGTTCCAAAGAGAGAAGGTAGAGTCTCTAAGAAGAAGCAAGAACACT |
| | GA |
| SEQ ID NO: | MEASEYGGFDHNAENEVKKMAKALVKENSNNRPLDSSSISRVTSGINPVGL |
| 1002 | QETDTGYNAKLDPNSNDFSSEEWTRNLANIVESEPEYYKPHSVGCCWKNLS |
| | AAGDSADLTYQTTFGNLPVKIFSLLYRHIRPSKGSDNFQILKPMDGLVDPGEL |
| | LVVLGRPGSGCTTLLKSISCNTHGFNVGKDSVIKYSGMASKDIRRNLRGEVA |
| | YNAESDTHIPNITVYQTLVTVARLKTPRNRIRGIDRESWAKHIAEVTMATYGLL |
| | HTRNTKVGNDLIRGVSGGERKRVSIAEATICGAKFQCWDNATRGLDSATALE |
| | FVRALRTQAKINNSTACVAIYQCSQHAYDLFDKVCVLYGGYQIYFGPTSNAKK |
| | YFESMGYYCPSRQATADFLTSITSPAERIVNREFLERNIVVPQTPDEMYLYW |
| | QNSPENHELQREIDSRLTQDYKENLTAIKASHNAAQSKRARKSSPYTVSYAM |
| | QVKYLLIRNMWRIINSPGVTLFRVFGNIIMSLVLGSMFYKVQKTTTTNTFYYRG |
| | AAMFYSILVNAFSSLIEIFALFEARPVTEKHKTYALYRPSADAFASFLSDIPGKV |
| | VSTVAFSIIYYFLVNFRRDAGRFFFYLLINLVTTFTMSHLFRCVGSISKTLTEAM |
| | VPASVLLLTLAVYTGFSIPRRSMHEWSKWISYIDPLSYLFESVMTNEFHGRNF |
| | PCAAYIPNGPQYQNTTGTERVCSVVGSKAGHDYVLGDDYLKQSYEYEIKHK |
| | WRGFGVGMAYVVFFFFVYLLICEYNEAAKQKGDLLVFPRSIVKKMRRQGTLQ |
| | KSNSEPEDVEKNATIIANVSPDSSLLLDTLESPSKQKSEIPLNQSDTIFHWRNV |
| | CYDVHIKKESRRILNNVDGWVKPGTLTALMGASGAGKTTLLDCLAKRVTTGVI |
| | TGEIFVNGKLRDESFPRSIGYCQQQDLHLKTATVRESLLFSAMLRQPKKVPLS |
| | EKKQYVEQIIEVLEMVPYADAIVGIEGEGLNVEQRKRLTIGVELVAKPKLLLFL |
| | DEPTSGLDSQTAWSICQLLRKLANRGQAVLCTIHQPSALLIREFDRLLFLQKG |
| | GKTVYFGELGDECKTMIDYFERNGAHKCPPDANPAEWMLEVVGAAPGTHA |
| | SHDYNEVWRNSEEYKEVQQELDRMESELKCVGEEDSSEKHQAFATDIFSQI |
| | LIVSHRFFQQYWRTPQYLWPKFILTAFDEIFIGFTFFKATRSLQGLQNQMLST |
| | FVFCVVYNALLQQFLPVYVEQRNLYEARERPSRTFSWFSFISSQILVEVPWNI |
| | IAGTIAFFVYYYPVGFYANASEANQLHERGALYWLFCTAFFVWIGSMGILANS |
| | FLEHAAEAANVALLCFAFSLAFCGVLVPPKLLPGFWIFMHRVSPLTYYIDSALS |
| | LGIANVDVKCSEIEYVKFTPPSNRTCGQYMQAYIKSIGTGYLADPSATNECNF |
| | CRLSKTNDYLKQISSSYSHRWRNYGIFICFIVFNYVAAVFLYWLARVPKREGR |
| | VSKKKQEH- |
| SEQ ID NO: | ATGTCTACCCCAAAGGATATCGACGTCAACTTGTCTAAATACGATACCGTT |
| 1003 | TTGTCCAACGACTCTCCATCTAATACTTACAACGGTTTCGATGACGAATCC |
| | AGAAAGTTGGTTAGAAACTTGGCTAAGACTCTGACCTCTAACTTCGAATCT |
| | ACTGAATCTTCTGCCTTGTCCTACAATGTTAGAGATACCAACGTTATGATC |
| | ATGAACCCATCTGAACCAGGTTACGATAAGAGATTGGATCCTAACTCTGA |
| | CGAGTTCTCTTCCAAATTGTGGATTCAATACTTGGCCCATTTGTCCTCTAC |
| | TGACCCAGAATATTACAAGCCATTTTCTTTCGGTTGTACCTGGAAGAACTT |
| | GTCTGTTTCTGGTGATTCTGCTGATGTTACTTACCAATCCACCATTTTCAA |
| | CGTCCCATTCAAGATTTTGGGCAAAGTTTACAGAAGGTTCAGACCAGCTA |
| | GAGACTCTAACTCTTTCCAAATTTTGAAGCCAATGGAAGGTTACTTGGACC |
| | CAGGTGAATTATTGGTTGTTTTGGGTAGACCAGGTTCTGGTTGTACTACTT |
| | TGTTGAAAACCATCTCCTCTAACACCCATGGTTTCAGAGTTGATAAGGATT |
| | CCGTTATCTCCTATAACGGTTTGACTCCAAGAGAAATGAGAAAGCACTTTA |
| | GAGGTGAAGTTGTTTACAACGCCGAATCCGATGTTCATTTGCCACATTTG |
| | ACTGTTTTCGAAACCTTATACACCGTCGCTAGATTGAAAACTCCAACTAAT |
| | AGAATCAAGGGTGTTGACAGAGATACCTACGCTAGACATATTACCGATGT |
| | TGCTATTGCTACTTACGGTTTGTCTCATACCAAGAACACTAAGGTTGGTAA |
| | CGCTTTGGTTAGAGGTGTTTCAGGTGGTGAAAGAAAGAGAGTTTCTATTG |
| | CCGAAGTTTCCATTTGCGGTTCTAAGTTTCAATGTTGGGATAACGCTACTA |
| | GAGGTTTGGATTCTGCTAATGCCTTGGAATTCATTAGAGCTTTGGATACC |
| | GAATCCTCTTTGCTAAAAACTGCTGCTGTTGTTGCTATCTACCAATGTTCT |
| | CAAACTGCCTACGATTTGTTCAACAAGGTTTGCGTTTTGAACAAGGGTTAC |
| | CAAATCTACTTCGGTCCAATTGATGAAGCTAAGGGTTACTTTGAATCCATG |
| | GGTTACAAATGCCCAGATAGACAAACTACTGCTGATTTCTTGACCTCTATC |
| | ACCAATCCATCTGAAAGAATCGTTAACCCAGAATTCATCGAAAAGGGTATT |
| | CCAGTTCCACAAACTCCAGACGAAATGTACACTTACTGGAAGTCATCTAG |
| | GGAATACGAAGAGTTGATGAAGAAGATCGACATCAGGTTGTCTGAAAACG |
| | AAGATGTTACCCGTAAGATGATGAAGTCATCACATGTTGCTAGACAGTCC |
| | AAGGGTATTAGATCTGGTTCTCCATATACTGTTAGATACGGCTTGCAAGTC |
| | AGATACTTGTTGACTAGAAACTTCTGGCGTATCCGTAACAACATTTCTGTT |
| | CCATTGGTTATGTTCATCGGCAATTCTTCCATGGCTTTCATTTTGGGTTCT |
| | ATGTTCTTCAAGGCCATGCAACAAGATAACACTACTACCTTTTATTTCAGA |
| | GGTGCTGCTATGTTTTTCGCTATCTTGTTCAACTCTTTCAGCTGCTTGTTG |
| | GAAATCTTCACCTTGTATGAAGCTAGACCAGTTTCCGAAAAACACAGAGC |
| | TTACTCATTATACCATCCTTCCGCTGATGCTTTCGCTTCTATTTTCTCTGAA |
| | TTGCCAAACAAGATCGTCATCTCCGTTGTGTTCAACATCATCTACTACTTC |
| | ATGGTCAACTTTAGAAGAACTGCTGGTGCTTTCTTTTTCTACTGGTTGATT |
| | TCTTTGGTTGGCGTTTTTGCCATGTCTCATTTGTTTAGAACTGTCGGTTCT |
| | TTGACCAAGACTTTGTCTGAAGCTATGGTTCCAGCTTCCATTTTGTTGTTG |
| | TCTATGTCTATGTACGCTGGTTTCGCTATTCCAAAGACTAAGATGTTAGGT |
| | TGGTCTAAGTGGATCTGGTACATTAACCCAATTGCCTACTTGTTCGAATCC |
| | TTGATGGTTAACGAATTCCACGGTAGAGAATTCCAATGCGCTAATTTCATT |
| | CCATCTGGTCCAACTTACTCTAATGCTACTGGTGACGAAAGATCTTGTTCT |
| | ACTTTGGGTGCTATTCCAGGTTCTTCTTATGTATTGGGTGACAACTACTTG |
| | AGACAGTCCTATGATTACTTGTACCAACATAAGTGGCGTGGTTTTGGTATT |
| | GGTTTGGCTTATGCTGTTTTCTTCTTGGTCGTTTACTTGATCGTTTGCGAA |
| | TTCAATGAAGGTGCTAAGCAAAAGGGTGAGATGTTGGTTTTTCCACATGG |
| | TGTTCTGAAGAAATTGAAAAAGAGGGGTGTTTTGTCCGATGATGATAAGA |
| | GAGATTTTGAGAAGGGTTCTTTCGATGCTACCAACCATGATTTGATTAAGG |
| | ACTCTGAATCCACCGACGAATCTTCTACTAATGGTGCTAGACTGTTGAAG |
| | TCTCAAGCTGTTTTTCATTGGAGGAACTTGTGCTACGATATTCCAATCAAA |
| | CACGGTACTAGAAGGTTGTTGGATAATGTTGATGGTTGGGTTAAGCCAGG |
| | TACTTTGACTGCTTTGATGGGTGCTTCTGGTGCTGGTAAAACTACTTTATT |
| | GGATTGCTTGGCTGAAAGGGTTACAATGGGTGTAATTACTGGTGATGTTT |
| | TGGTCGATGGTAGACCAAGAGATGAATCTTTCCCAAGATCTATTGGTTAC |
| | TGCCAACAACAAGACTTGCACTTGAAAACTTCTACCGTCAGAGAATCTTTG |
| | AGATTCTCAGCCTATTTGAGACAACCAGCTGAAGTTTCTGTTGAAGAAAA |
| | GGATGCTTACGTCGAAGAGGTCATTAAGATATTGGAGATGGAAAAGTACG |
| | CTGATGCAGTTGTTGGTGTTGCTGGTGAAGGTTTGAATGTCGAACAAAGA |
| | AAAAGATTGACCATCGGTGTTGAATTGGCTGCTAAACCTAAGTTGTTAGTT |
| | TTCTTGGACGAACCTACTTCTGGTTTGGATAGTCAAACTGCTTGGTCTATC |
| | TGTCAGTTGATGAGAAAATTGGCATCACATGGTCAAGCTATTTTGTGCACT |
| | ATTCATCAACCATCCGCCATCTTGATGCAAGAATTTGATAGATTGCTGTTC |
| | TTGCAGGATGGTGGTCAAACAACTTACTTTGGTGAATTAGGTGATGGTTG |
| | CTGCACTATGATTGACTACTTTGAAAGAAACGGTGCTCATAAGTGTCCAAT |
| | TGGTGCTAATCCAGCTGAATGGATGTTGGAAGTTGTCGGTGCTGCTCCAG |
| | GTTCACAAGCTACTCAAGATTACTTTAAGATCTGGCGTAACTCCGAAGAAT |
| | TCAAGGCTGTTCACAAAGAATTGGACAGCTTGGAAAAAGAGTCTAACTTA |
| | AGACCAGAAGGTATCACTACTGATCATGCTGAATTCGCTACTTCCATTCCA |
| | TACCAAATCAGATTGGTTTCTGCTAGGTTGTTCCAACAGTACATTAGGGCT |
| | CCAGAATACTTGTGGTCTAAATTTGGTTTGACCATTGTCGACGAGTTGTTC |
| | ATTGGTTTCACTTTCTTTAAGGCCGGTACTAGCTTGCAAGGTCTACAAAAT |
| | CAAATGTTGGCTGCTTTCATGTTCACCGTTGTTTTTAACCCACTGTTGCAG |
| | CAATACTTGCCATCTTTCGTTCAACAACGTGACTTATACGAAGCTAGAGAA |
| | AGACCATCTAGAACCTTTTCATGGAAGGCTTTCATCGTTTCCCAAATCTTG |
| | GTTGAAGCTCCATGTAATTTCTTGGCTGGTACTTTGGCTTACTTCATCTAT |
| | TACTACCCAATCGGTTTCTACGAAAACGCTTCTTTTGCTGGTCAATTGCAC |
| | GAAAGAGGTGCTTTGTTTTGGTTGTTCTCTACAGGCTTTTACGTTTTCGTT |
| | GGTTCTATGGGTTTCCTGACTGTCTCTTTTAATGAAGTTGCTCAAAATGCT |
| | GCCGGTATTGCTTCTTTGATGTTCGTTATGTGTACTACCTTCTGTGGTGTT |
| | TTGGCTACTCCAGAAGTTATGCCAGGTTTTTGGATTTTCATGTACAGATTG |
| | TCTCCCTTGACCTACTTCGTTCAAGGTTTCTTAGCTACAGGTTTGGCTAAC |
| | GCTAAAATCCAGTGTTCGGAGAAAGAGTTCATTGTCTTTTCTCCACCATCT |
| | GGTATGAATTGTGGTCAGTACATGGAACAGTACATCACTAATACTGGTAC |
| | TGGCTACTTGGAAGATTCTGAATCTACATCTACCTGCGAATTCTGCCAATT |
| | CTCTTACACTAATGACTTTTTGGCCTCCGTCAACTCCTTTTATTCTCAACG |
| | TTGGAGGAATTGGGGTATCTTCATTTGTTACATTGCCTTCAATTATATGGC |
| | CGGCATTTTCTTGTATTGGTTGGCAAGAGTTCCAAAGAAGTCCTCTGGTTT |
| | GAAAAGAAAGATCCGTAAGAACGAGCTGTCCTGA |
| SEQ ID NO: | MSTPKDIDVNLSKYDTVLSNDSPSNTYNGFDDESRKLVRNLAKTLTSNFEST |
| 1004 | ESSALSYNVRDTNVMIMNPSEPGYDKRLDPNSDEFSSKLWIQYLAHLSSTDP |
| | EYYKPFSFGCTWKNLSVSGDSADVTYQSTIFNVPFKILGKVYRRFRPARDSN |
| | SFQILKPMEGYLDPGELLVVLGRPGSGCTTLLKTISSNTHGFRVDKDSVISYN |
| | GLTPREMRKHFRGEVVYNAESDVHLPHLTVFETLYTVARLKTPTNRIKGVDR |
| | DTYARHITDVAIATYGLSHTKNTKVGNALVRGVSGGERKRVSIAEVSICGSKF |
| | QCWDNATRGLDSANALEFIRALDTESSLLKTAAVVAIYQCSQTAYDLFNKVC |
| | VLNKGYQIYFGPIDEAKGYFESMGYKCPDRQTTADFLTSITNPSERIVNPEFIE |
| | KGIPVPQTPDEMYTYWKSSREYEELMKKIDIRLSENEDVTRKMMKSSHVARQ |
| | SKGIRSGSPYTVRYGLQVRYLLTRNFWRIRNNISVPLVMFIGNSSMAFILGSM |
| | FFKAMQQDNTTTFYFRGAAMFFAILFNSFSCLLEIFTLYEARPVSEKHRAYSL |
| | YHPSADAFASIFSELPNKIVISVVFNIIYYFMVNFRRTAGAFFFYWLISLVGVFA |
| | MSHLFRTVGSLTKTLSEAMVPASILLLSMSMYAGFAIPKTKMLGWSKWIWYIN |
| | PIAYLFESLMVNEFHGREFQCANFIPSGPTYSNATGDERSCSTLGAIPGSSYV |
| | LGDNYLRQSYDYLYQHKWRGFGIGLAYAVFFLVVYLIVCEFNEGAKQKGEML |
| | VFPHGVLKKLKKRGVLSDDDKRDFEKGSFDATNHDLIKDSESTDESSTNGAR |
| | LLKSQAVFHWRNLCYDIPIKHGTRRLLDNVDGWVKPGTLTALMGASGAGKTT |
| | LLDCLAERVTMGVITGDVLVDGRPRDESFPRSIGYCQQQDLHLKTSTVRESL |
| | RFSAYLRQPAEVSVEEKDAYVEEVIKILEMEKYADAVVGVAGEGLNVEQRKR |
| | LTIGVELAAKPKLLVFLDEPTSGLDSQTAWSICQLMRKLASHGQAILCTIHQPS |
| | AILMQEFDRLLFLQDGGQTTYFGELGDGCCTMIDYFERNGAHKCPIGANPAE |
| | WMLEVVGAAPGSQATQDYFKIWRNSEEFKAVHKELDSLEKESNLRPEGITTD |
| | HAEFATSIPYQIRLVSARLFQQYIRAPEYLWSKFGLTIVDELFIGFTFFKAGTSL |
| | QGLQNQMLAAFMFTVVFNPLLQQYLPSFVQQRDLYEARERPSRTFSWKAFI |
| | VSQILVEAPCNFLAGTLAYFIYYYPIGFYENASFAGQLHERGALFWLFSTGFY |
| | VFVGSMGFLTVSFNEVAQNAAGIASLMFVMCTTFCGVLATPEVMPGFWIFM |
| | YRLSPLTYFVQGFLATGLANAKIQCSEKEFIVFSPPSGMNCGQYMEQYITNT |
| | GTGYLEDSESTSTCEFCQFSYTNDFLASVNSFYSQRWRNWGIFICYIAFNYM |
| | AGIFLYWLARVPKKSSGLKRKIRKNELS- |
| SEQ ID NO: | ATGGTGTCCTCTGATACCGATTCCATCGTTGTTAAGTCTGATGCTGGTTCT |
| 1005 | GATGTTGCTTCTTACAGAGGTTTTGATGCTACTGCTGATGAACAAGTTCAT |
| | GATTTGGCTAGAAAGTTGACCAACGAATCTGGTCATTTGTCTTTGTTTTTG |
| | GCTGATGGTAAACCTAGACCATCCGTTGTTTCTGATTCTGCTCATTTGGTT |
| | AGAACCTTGTCTACCATGTCTAATGTTCCAGGTGTTTCTACTTTCACCGAA |
| | GGTGAAATTGATTCCAGATTGGACCCAAACTCTGATGATTTCGATTCTAAG |
| | TTGTGGGTCAAGAACTTCCGTAAGTTGATGGATAATGATCCCGATTACTA |
| | CAAGCACACCTCTTTGGGTATTGCTTACAGAAATTTGAGAGCTTCTGGTAT |
| | TGCTGCTGATGCTTCTTATCAACCTACCATTTTGAACTACCCATACAAGGT |
| | TGTTGAGGACTTGTACAACAAGTTCGCTAAAGAAAATCCAGCCAGATTCTT |
| | CGATATCTTGAAACCTATGGATGCCATTATGAAGCCAGGTTCTTTGACTGT |
| | TGTTTTGGGTAGACCAGGTGCTGGTTGTTCTACTTTGTTGAAAACTATTTC |
| | CGCTCAGACCTACGGTTTTAAGATTGCTCCAGAATCCGAAATCTCTTACG |
| | ATGGTTTTACCATGAACGAGATCAACAAACACTACAGAGGTGAAGTTGTTT |
| | ACTCCGCTGAAGTTGATAATCATTTCCCACATTTGACAGTCGGTCAAACTT |
| | TGAATTTTGCTGCTGGTTTGAGAACCCCACAAAACAGAGGTAATGGTATC |
| | AACAGAGAAGTTTACGCTAAGCACATGACCGATGTTTACATGGCTACTTAT |
| | GGTTTGTTGCATACCAGAAACACTAAGGTTGGTAACGATTTGGTAAGAGG |
| | TGTTTCAGGTGGTGAAAGAAAGAGAGTTTCTATTGCCGAAGTTTCTTTGTG |
| | TGGTTCTGCTTTACAATGTTGGGATAATGCTACTAGAGGTTTGGACGCTG |
| | CTACAGCTTTGGAATTCATTAGAGCTTTGAAAACTTCCGCCGTTTTGTTGG |
| | ATACAACTCCATTGATTGCTATCTACCAATGCTCTCAAGATGCCTACGATT |
| | TGTTTGATAACGTCATCGTCTTGTACGAAGGTTACCAGATCTATTTTGGTT |
| | TGGGTACTAAGGCCAAGGACTACTTTTTGAGAATGGGTTATGATTGCCCA |
| | CCAAGACAAACTACAGCTGATTTCTTGACTTCTCTGACTAACCCATCTGAA |
| | AGAGTTGCTAAACCAGGTTTCGAAAACAAGGTTCCAAAGACTCCAAAAGA |
| | GTTCTCCGATTATTGGAGAAACTCCCCAGAATTCGTTGAATTGACTGAAG |
| | AAGTTGACGCCTACATCCAAGATCATAAGGTTAACAATGCTACCACCGAA |
| | TTCTTGGAATCTAAGGTTGCTAGACAATCCAATCATACCAGACCAACTTCA |
| | TCTTACACCGTGTCTTATTCCATGCAAATCAAGGCCATTTTGAGAAGAAAT |
| | TGGTGGCGTTTACAAGGTGATCCATCTATTACTCTGTTCTCCGTTATCGCT |
| | AACACCATAATGGGTTTGTTGTTGTCCTCTCTGTTTTACAATTTGCCAGCT |
| | ACTTCTGGCTCTTTCTATTACAGATCTGCTTCTTTGTTCTTCGCCGTCTTG |
| | TTTAATGCTTTCAGCTCTTTGTTGGAAGTCATGGCTTTGTTTGAATCCAGA |
| | CCAATTATCGAGAAGCACAAAAAGTATGCCTTGTACCATCCATCAGCAGA |
| | TGCTATGGCTTCCATTATTTCTGAATTTCCACCAAAGTTGATTACCGCCGT |
| | TGGTTTTAACTTGGTCTTTTACTTCATGGTGAACTTCAGAAGAAACCCAGG |
| | TAGATTCTTCTTCTACTTCCTGGTTAATTTCTTGGCTACCTTGGCTATGTC |
| | CCATATCTTTAGATCTATCGGCTCTTTTTACAAGACCTTGTCTGAAGCTAT |
| | GTCTTTGGCTGCTTTGATTTTGTTGGCTTTGGTTATCTACACCGGTTTCGT |
| | TGTTCCAACTCCATCTATGTTAGGTTGGTCTAGATGGATTAACTACTTGGA |
| | TCCTATTGCCTACGTGTTCGAATCTTTGATGACAAACGAATTCCAAGACAG |
| | GTTGTTCGAATGCTCTGCTTTTATTCCATCTGGTGAAGCTTACTCTAAATA |
| | CCCATCTGCTAACAAGGTCTGTTCCTCTGTTTCTTCAGTTGCTGGTGAAG |
| | ATTTTGTCAACGGTACTAAGTATGTTAACGCTGCTTATGCCTACTACAACA |
| | AGCACAAATGGCGTAATGTTGGTGTTGTTATTGCCTTCGTTGTTTTCTTCT |
| | TGGGTGTTTACTTGGCCATTTGCGAATTGTCTAGAGGTTCATTGCAAAAG |
| | GGTGAAGTCTTGGTTTTCCAATCTTCTACCTTGAGGAAATTGAAAAAGCAA |
| | AACAAGCTGATCAACTCCGACGATCCAGAATCTGCTTTGAATGGTGAAAA |
| | GCAAGCCTCCGTTATAGAAGAAACTGGTTCTACTTCTGAGGACAAAGGTG |
| | GTGCTGCTGTTAAGTTGTCTGCTGGTAAAGATATTTTCCATTGGAGAGAT |
| | GTTTGCTACGAGGTTAACATTAAGACCGAAGTCAGAAAGATCTTGAACCA |
| | TGTTGATGGTTGGGTTAAGCCAGGTACATTAACTGCTTTAATGGGTGCTT |
| | CTGGTGCAGGTAAAACTACCTTGTTGGATGTTTTGGCTAACAGAGTTACT |
| | ATGGGTGTTGTAACCGGTTCTATGTTTGTTAACGGTAGATTGAGAGATGA |
| | CTCCTTCCAAAGATCTACTGGTTACGTTCAACAACAAGACTTGCACTTGCA |
| | AACTGCTACTGTTAGAGAATCCTTGAGATTCTCTGCTTACTTGAGACAACC |
| | AGCTTCTGTTTCTCAAGCTGAAAAGGATGAATACGTCGAAAACGTGATCG |
| | AAATCTTGGAGATGGAAAAGTACGCTGATGCTATAGTTGGTGTTAGTGGT |
| | GAAGGTTTGAACGTTGAACAGAGAAAAAGATTGACCATCGGTGTTGAATT |
| | GGCTGCTAAACCTCAACTGTTGTTGTTCTTGGATGAACCTACATCTGGTTT |
| | GGATTCTCAAACTGCTTGGTCTATTTGCCAGTTGATGAGAAAGTTGGCTG |
| | ATAACGGTCAAGCTATTTTGTGCACTATTCATCAACCATCCGCTATCTTGT |
| | TGCAAGAATTTGACAGATTGCTGTTCTTAGCTAGAGGTGGTAGAACTGTTT |
| | ACTTTGGTGATTTGGGTGAAAACTGTACCACCTTGATTGACTACTTCGAAA |
| | AACATGGTGCTCATCCATGTCCACATGATGCTAATCCAGCTGAATGGATG |
| | TTAGAAGTTATTGGTGCTGCACCAGGTTCTCATGCTAATCAAGATTATCAT |
| | GAAGTCTGGATGAACTCTGCTGAAAGAGCTGCAGTTAGAACTGAATTAGC |
| | TACCATGGAACACGAATTGGTTAAGATCCCAAAAGATGATTCTCCAGACG |
| | CCAGAAAAGAATTTGCTGCTCCATTGTGGTTTCAGTACACTCAAGTTACTA |
| | GAAGGGTTTTCGAACAGTACTTCAGAACTCCAACTTACATTTGGTCCAAG |
| | CTGTTGTTGACTATTATCTCCGCTATTTTCAACGGCTTCTCATTTTTCAAG |
| | GCTGGTTTGTCATTGCAAGGCTTGCAGAATCAAATGTTGGCTATCTTCAT |
| | GTTCCTGATCGTTTTGATGACTTTGACCCAACAATACTTGCCCAACTACGT |
| | TTTTCAAAGGGGTCTATATGAAGCTAGAGAAAGACCATCTAAGACCTTTTC |
| | TTGGTTGGCTTTCATTTTGGCCCAAATTACCGTTGAAATTCCATGGCAGAT |
| | GTTGTGTGGTACTTTGTCATTTTTCTGTTGGTACTACCCAGTCGGTATGTA |
| | CGAAAATGCTGTTGCTACTGATACTGTTCACGAAAGAGGTGCTTTAATCT |
| | GGTTGTATATTGTCGCCTTTTTCGTTTACGCTTCCACTTTGGCTCAATTGT |
| | GTGTTGCAGGTATGGAAATTGCTGATAATGCTGCTAATTTGGCCTCTCTG |
| | ATGTTCACAATGTCCTTGAATTTCTGCGGTATCTTGAAGTACCCATCCGGT |
| | TTTTGGATTTTCATGTACAGAGTTTCCCCATTCACTTACTGGGTTCAAGGT |
| | GTTTTGTCTACTGGTGTTGGTAAATCCAAGGTTACTTGTTCTACCTCCGAA |
| | TACGTTCATTTTCCACCACCATCAGGTATGAATTGTGGTGATTATATGAAG |
| | GACTACATTTCTGCAGCTGGTGGTTACTTATTGGATGAAGATGAGACTTCT |
| | AAGTGCTCTTTCTGTTCTACTGCTTCTACTGATGTCTACTTGGCTTCTGTT |
| | AACTCCTACTATTCTCAGCGTTGGAGAAATTACGGTATCTACATTGCCTTC |
| | ATTTTCATCAACATCTTCGGCACCGTTTTCTTGTATTGGTTGGTTAGAGTT |
| | CCCAAGTCCAAGAATAGAGTTAAGGATGAAGCTCCATCAGCTGAAGAAGA |
| | GGAACAAAAGACTATCGAAAGACAGTTGTCTAGAACCCAATCCAGAAACT |
| | CCAAAAAGTCCAGAAAGTCGATCATCTAA |
| SEQ ID NO: | MVSSDTDSIVVKSDAGSDVASYRGFDATADEQVHDLARKLTNESGHLSLFLA |
| 1006 | DGKPRPSVVSDSAHLVRTLSTMSNVPGVSTFTEGEIDSRLDPNSDDFDSKL |
| | WVKNFRKLMDNDPDYYKHTSLGIAYRNLRASGIAADASYQPTILNYPYKVVE |
| | DLYNKFAKENPARFFDILKPMDAIMKPGSLTVVLGRPGAGCSTLLKTISAQTY |
| | GFKIAPESEISYDGFTMNEINKHYRGEVVYSAEVDNHFPHLTVGQTLNFAAGL |
| | RTPQNRGNGINREVYAKHMTDVYMATYGLLHTRNTKVGNDLVRGVSGGER |
| | KRVSIAEVSLCGSALQCWDNATRGLDAATALEFIRALKTSAVLLDTTPLIAIYQ |
| | CSQDAYDLFDNVIVLYEGYQIYFGLGTKAKDYFLRMGYDCPPRQTTADFLTS |
| | LTNPSERVAKPGFENKVPKTPKEFSDYWRNSPEFVELTEEVDAYIQDHKVNN |
| | ATTEFLESKVARQSNHTRPTSSYTVSYSMQIKAILRRNWWRLQGDPSITLFS |
| | VIANTIMGLLLSSLFYNLPATSGSFYYRSASLFFAVLFNAFSSLLEVMALFESR |
| | PIIEKHKKYALYHPSADAMASIISEFPPKLITAVGFNLVFYFMVNFRRNPGRFF |
| | FYFLVNFLATLAMSHIFRSIGSFYKTLSEAMSLAALILLALVIYTGFVVPTPSML |
| | GWSRWINYLDPIAYVFESLMTNEFQDRLFECSAFIPSGEAYSKYPSANKVCS |
| | SVSSVAGEDFVNGTKYVNAAYAYYNKHKWRNVGVVIAFVVFFLGVYLAICEL |
| | SRGSLQKGEVLVFQSSTLRKLKKQNKLINSDDPESALNGEKQASVIEETGST |
| | SEDKGGAAVKLSAGKDIFHWRDVCYEVNIKTEVRKILNHVDGWVKPGTLTAL |
| | MGASGAGKTTLLDVLANRVTMGVVTGSMFVNGRLRDDSFQRSTGYVQQQD |
| | LHLQTATVRESLRFSAYLRQPASVSQAEKDEYVENVIEILEMEKYADAIVGVS |
| | GEGLNVEQRKRLTIGVELAAKPQLLLFLDEPTSGLDSQTAWSICQLMRKLAD |
| | NGQAILCTIHQPSAILLQEFDRLLFLARGGRTVYFGDLGENCTTLIDYFEKHGA |
| | HPCPHDANPAEWMLEVIGAAPGSHANQDYHEVWMNSAERAAVRTELATME |
| | HELVKIPKDDSPDARKEFAAPLWFQYTQVTRRVFEQYFRTPTYIWSKLLLTIIS |
| | AIFNGFSFFKAGLSLQGLQNQMLAIFMFLIVLMTLTQQYLPNYVFQRGLYEAR |
| | ERPSKTFSWLAFILAQITVEIPWQMLCGTLSFFCWYYPVGMYENAVATDTVH |
| | ERGALIWLYIVAFFVYASTLAQLCVAGMEIADNAANLASLMFTMSLNFCGILKY |
| | PSGFWIFMYRVSPFTYWVQGVLSTGVGKSKVTCSTSEYVHFPPPSGMNCG |
| | DYMKDYISAAGGYLLDEDETSKCSFCSTASTDVYLASVNSYYSQRWRNYGIY |
| | IAFIFINIFGTVFLYWLVRVPKSKNRVKDEAPSAEEEEQKTIERQLSRTQSRNS |
| | KKSRKSII |
| SEQ ID NO: | ATGACCTCCATCCACGATATTGAAGATGCTAGTGCTGAAGATGTTAACAG |
| 1007 | ATACGATGGTTACACTAACACCGTTGATTCTGCTGTTACTGAATTGGCTAG |
| | ACAAATCACCAACCACTCTCAATCTTCATTTCAAGATGTCCCATACAAATT |
| | GGCTGCTGGTGAATCTGAACAAGATGCTTTGTCTAGAGTTTCCACTATTG |
| | CTCAAGGTGTTAACCCAATGTCTGACATGTCTAATATCGATCCAAGATTGG |
| | ACCCAAACTCCGATGAATTCAATTCTAGATACTGGATCAAGAACTTCAAGG |
| | CCTTAATGGATAAGGATCCAGATCACTACCAAAACTACTCATTGGGTATTG |
| | CCTTCAAGGATTTGAGAGCTTATGGTGATGCTACTGGTGCTGATTATCAA |
| | ACTACTACTTTGAACGCTCCAATGAAGTTCGCTGTTCAATACGCTAAGGAT |
| | ATCTTCTCATCTAAGGCTGCTAAAATGGCCAACAAGTTCGATATTTTGAAG |
| | CCATTGGATGGCATTATCAAGCCAGGTGAAGTTGTTGTTGTTTTGGGTAG |
| | ACCAGGTGCTGGTTGTACTACTTACTTGAAAACTATTGCTGCTAACACCCA |
| | CGGTTTTGATGTTGGTGAAGAATCCGAAATTTCTTACGACGGTTTGACCT |
| | CTTCCGAAATCAAGAAACATTTTAGAGGTGAGGTTGTCTACAACGCCGAA |
| | TCTGATATTCATTTCCCACATTTGACTGTCTGGCAAACTTTGACTACAGCT |
| | GCTAAATTCAGAACCCCAGAAAACAGAATTCCAGGTGTTTCTAGAGAACA |
| | ATACGCTGAAGCTTTGACCAATGTTTACATGGCTACTTATGGTTTGTCTCA |
| | CACTAAGAACACTAAGGTCGGTTCTGAAATAGTTAGAGGTGTTTCAGGTG |
| | GTGAAAGAAAGAGAGTTTCTATTGCCGAAGTTTCTTTGGCTGGTGCTAGA |
| | TTGCAATGTTGGGATAATGCTACTAGAGGTTTGGATTCTGCTACTGCCTT |
| | GGAATTCATTAGAGCTTTGAGAACTCAAGCCGATGTTTTGGATACAACTG |
| | CTTTGATTGCTATCTACCAATGCTCTCAAGATGCCTACGATTTGTTCGATA |
| | AGGTTTCTGTCTTGTACGAAGGTTACCAAATCTTTTTCGGTAAGGCTGATA |
| | AGGCTAAAGAATACTTCGTTAACATGGGTTGGGATTGTCCAGCTAGAGCT |
| | ACTACTGCTGATTTCTTGACTTCTGTTACTTCCCCAAGAGAAAGAGAACCT |
| | AGAGCTGGTTTTGAAAACAAGGTTCCAAAAACTCCAGAAGAGTTCTCTAT |
| | CTATTGGAGGAATTCTCCAGAAAGAGCCGAGTTGTTGAAAGAAGTTGAAA |
| | CTACTTTGGCTCAGAACAACAATTCCGAATTGAAGCAATCTATCTACGATG |
| | CCAAACACTCTAGGCAATCTAAGAGAATGAGATCCTCTTCTCCATTCACC |
| | GTTTCTGTTTTGTTGCAGACTAAGTACTTGTTAGCCAGAGAAGTCTACAGG |
| | ATTAAGAATGATTGGGGTTTCCACTTCTTCTCTATGGGTGCTAATTCTTTG |
| | ATGGCCTTGGTTTTGTCCTCCATCTTTTACAATTTGCCATCTACCACCTCG |
| | TCCTTTTATTACAGAGGTGCTGCTATGTTTTTCGCCTGTTTGTTTAATGGC |
| | TTCTCGTCCTTCTTGGAGATCTTGTCTTTGTTTGAAGCTAGGCCAATTATC |
| | GAAAAGCACAAGCAATATGCCTTGTACCATCCATCTGCTAATGCTTTGGC |
| | TTCCGTTATTTCTCAATTGCCATTCAAAGCTTTCACCGCCTTGTTTTTCAAC |
| | CTGATCTTTTACTTCATGGTCAACTTCAGAAGAAACCCAGGTAGATTCTTC |
| | TTCTACATGTTGGCTAATGTTACTGCTACCTTGACCATGTCTCACTTTTTC |
| | AGATTGATTGGTTCTGCTGCCTCATCATTGCAAGAAGCTGTTGTTCCAGG |
| | TCACATAGTTTTGTTGGGTTTAGCTATGTTCGTTGGTTTCACTTTGCCAGT |
| | CGATTATATGTTAGGTTGGTGTAGATGGATCAACTACATTAACCCATTGGC |
| | CTATTCTTTCGAAGCCTTGATGGCTAACGAATTCCATGATAGAGAATTCGG |
| | TTGTTCAGCTTATTTGCCAGGTGATCCAGCTGATCATCCATCTTGGTCATC |
| | TGATTCTTGGATCTGTAATTCTGTTGGTGCTGTTGCTGGTGAGTATACTGT |
| | TTCTGGTGATAGGTACATTGAGTTGGCTTACTCTTACAAGAACACTCATAA |
| | GTGGCGTAACTTCGGTATTTCCATTGCTTTCATGATCTTTTTCTTGGTGTT |
| | CTACATGGTGTTCTCCGAGTATAATGAATCCGCTAAACAAAAGGGCGAGA |
| | TCCTGTTGTTTCAAAGGTCTAATTTGAAGAAGATTAAGAAAGAAAAGGGC |
| | GCCATCAATGATGTTGAAGCTGGTAATGGTAGAGAAGTTGTTTCCCAAGA |
| | TGAATCCGAAGAACAACAGGTTGATGCTATTCAAGCTGGTACTGATATTTT |
| | CCATTGGAGAGATGTTCACTACACCGTCAAAATCAAATCCGAGTACAGAG |
| | AAATCTTAGGTGGTGTTGACGGTTGGGTTAAGCCAGGTACTTTGACTGCT |
| | TTAATGGGTGCTTCTGGTGCAGGTAAAACAACTTTGTTGGATGTTTTAGCC |
| | TCCAGAGTTACTATGGGTGTTGTTACTGGTAACATGTTCGTTAACGGTAG |
| | ACTGAGAGATTCCTCATTCCAAAGATCTACTGGTTACGTTCAACAACAAGA |
| | CTTGCATTTGTCTACCGCTACTGTTAGAGAAGCCTTAAGATTTTCTGCCTA |
| | CTTGAGACAACCAGCCTCTGTTTCTAAAGCTGAAAAGGATGCTTACGTCG |
| | AAGAATGCATTAAGATCTTGGACATGCAAAAGTACGCTGATGCTATAGTT |
| | GGTGTTGCCGGTGAAGGTTTAAATGTCGAACAAAGAAAAAGGTTGACCAT |
| | CGGTGTTGAATTAGCTGCTAAGCCAAAGTTGCTGTTATTCTTCGATGAAC |
| | CTACATCTGGTTTGGACTCTCAAACTGCTTGGTCTATTTGTCAGTTGATGA |
| | GAAAGTTGGCTAACCACGGTCAAGCTATTTTGTGTACTATTCATCAACCAT |
| | CCGCCATCTTGATGCAAGAATTTGATAGGTTGCTGTTCTTGGCTAGAGGT |
| | GGAAGAACTGTTTATTTCGGTGATTTGGGTAAGAACTGCCAAGTTTTGATC |
| | GACTACTTCGAATCTCATGGTGCTCCAAAATGTCCAGGTGATGCAAATCC |
| | AGCAGAATGGATGTTGCAAGTTATTGGTGCTGCTCCAGGTTCTCATGCTA |
| | ATCAAGATTATCATCAAGTCTGGCTGAACTCCAAAGAAAGACAGGCTGTT |
| | TTAGATGAGTTGGACTCTATGGAAAGAGAGCTAGTTTCAATTCCTTACGAT |
| | GGTGATTCTAAGCACTCTGAATTTGCTGCACCATTTTACGTTCAGTTGTAC |
| | GTTGTTACCGAAAGGGTGTTTCAAGAATTTTGGAGAACCCCATCTTACATT |
| | TGGGCCAAGATGTTCTTGTCCTCTATCTCCTCTTTGTTCATCGGCTTCATT |
| | TTCTTCAAGGCTAAAAACACCATTCAGGGCTTGCAAAATCAGATGTTCGCT |
| | CTGTTTATGTTCCTGGTCGTTTTTAACCCTTTGCTGCAACAGACTTTGCCA |
| | ACCTTTGTTAATCAGAGAAACCTGTACGAAACCAGAGAAAGACCAGCTAA |
| | AACTTTTGCTTGGCAAGCCTTCATTCTGTCCCAAATTATTGCTGAAATCCC |
| | CTGGAATATCTTCGTTGGTACTTTAGGTTTCTTTTGCTTCTATTACCCACC |
| | AGCTTTTTACCAAAATGCCGAACCATATCATGAAGTTAATGCAAGAGGTG |
| | CTTACGCCTGGTTTTTCTCTATTTTGTTCTACGTCTACATCGGTACTATGG |
| | CTCATATGTGTATGGCTCCATTGGATTTGGTTGATTCAGCTGGTAACTTGG |
| | GTTCTTTGTTGTTCACTATGTGCTTGAATTTCTGCGGTGTTTTGGTCACAA |
| | AAGAAGCTATGCCAGGTTTTTGGGTTTTCATGTATAGAGTTTCACCCTTCA |
| | CCTACTACATCGAAGGTTTCTTAACTAACGCTGTTGCCCATAACGATATTG |
| | TCTGTGCTGATAATGAATACCGTGTATTGGTTCCACCAACTGGTGAAACTT |
| | GTGAAGATTACTTGTCCGATTACATTGCCTCACAAGGTACAGGTTATATCT |
| | TAGATCCATCCGCTACAGATTCTTGTTCTTTGTGTCCAATGTCCTCTACCG |
| | ATGATTTCATTGCTTCCCTGAAATTGAACTACGATAACAGATGGCGTGATG |
| | TCGGTATCTTTATCGCTTTCATCTTTTTCAATATGATCATGGCCGTGTTCTT |
| | TTACTGGTTAGCTAGAGTTCCTAAGAAGTCCGATAGAGTTGGTACAGAAC |
| | AACCTAAAGAAGCCGTTAATATGGGTGCACAAATGGAAAACAATGCTATG |
| | AATGCTCACAAGCACGATGATGTTGTTCAAGACGAAAAATTGGACGAAGG |
| | TTCCATTGAAAAGGGTGAGAATTCTGATGTCTCCAGGTAA |
| SEQ ID NO: | MTSIHDIEDASAEDVNRYDGYTNTVDSAVTELARQITNHSQSSFQDVPYKLA |
| 1008 | AGESEQDALSRVSTIAQGVNPMSDMSNIDPRLDPNSDEFNSRYWIKNFKAL |
| | MDKDPDHYQNYSLGIAFKDLRAYGDATGADYQTTTLNAPMKFAVQYAKDIFS |
| | SKAAKMANKFDILKPLDGIIKPGEVVVVLGRPGAGCTTYLKTIAANTHGFDVG |
| | EESEISYDGLTSSEIKKHFRGEVVYNAESDIHFPHLTVWQTLTTAAKFRTPEN |
| | RIPGVSREQYAEALTNVYMATYGLSHTKNTKVGSEIVRGVSGGERKRVSIAE |
| | VSLAGARLQCWDNATRGLDSATALEFIRALRTQADVLDTTALIAIYQCSQDAY |
| | DLFDKVSVLYEGYQIFFGKADKAKEYFVNMGWDCPARATTADFLTSVTSPRE |
| | REPRAGFENKVPKTPEEFSIYWRNSPERAELLKEVETTLAQNNNSELKQSIY |
| | DAKHSRQSKRMRSSSPFTVSVLLQTKYLLAREVYRIKNDWGFHFFSMGANS |
| | LMALVLSSIFYNLPSTTSSFYYRGAAMFFACLFNGFSSFLEILSLFEARPIIEKH |
| | KQYALYHPSANALASVISQLPFKAFTALFFNLIFYFMVNFRRNPGRFFFYMLA |
| | NVTATLTMSHFFRLIGSAASSLQEAVVPGHIVLLGLAMFVGFTLPVDYMLGW |
| | CRWINYINPLAYSFEALMANEFHDREFGCSAYLPGDPADHPSWSSDSWICN |
| | SVGAVAGEYTVSGDRYIELAYSYKNTHKWRNFGISIAFMIFFLVFYMVFSEYN |
| | ESAKQKGEILLFQRSNLKKIKKEKGAINDVEAGNGREVVSQDESEEQQVDAI |
| | QAGTDIFHWRDVHYTVKIKSEYREILGGVDGWVKPGTLTALMGASGAGKTTL |
| | LDVLASRVTMGVVTGNMFVNGRLRDSSFQRSTGYVQQQDLHLSTATVREAL |
| | RFSAYLRQPASVSKAEKDAYVEECIKILDMQKYADAIVGVAGEGLNVEQRKR |
| | LTIGVELAAKPKLLLFFDEPTSGLDSQTAWSICQLMRKLANHGQAILCTIHQPS |
| | AILMQEFDRLLFLARGGRTVYFGDLGKNCQVLIDYFESHGAPKCPGDANPAE |
| | WMLQVIGAAPGSHANQDYHQVWLNSKERQAVLDELDSMERELVSIPYDGD |
| | SKHSEFAAPFYVQLYVVTERVFQEFWRTPSYIWAKMFLSSISSLFIGFIFFKAK |
| | NTIQGLQNQMFALFMFLVVFNPLLQQTLPTFVNQRNLYETRERPAKTFAWQA |
| | FILSQIIAEIPWNIFVGTLGFFCFYYPPAFYQNAEPYHEVNARGAYAWFFSILFY |
| | VYIGTMAHMCMAPLDLVDSAGNLGSLLFTMCLNFCGVLVTKEAMPGFWVFM |
| | YRVSPFTYYIEGFLTNAVAHNDIVCADNEYRVLVPPTGETCEDYLSDYIASQG |
| | TGYILDPSATDSCSLCPMSSTDDFIASLKLNYDNRWRDVGIFIAFIFFNMIMAV |
| | FFYWLARVPKKSDRVGTEQPKEAVNMGAQMENNAMNAHKHDDVVQDEKL |
| | DEGSIEKGENSDVSR- |
| SEQ ID NO: | ATGTCCCTGAACTCCAACTCCATTACCAATAGATCTGTTGTCTCTATGAAC |
| 1009 | CCATCCGAACCTATTTCTGAAACCGTTTCTCAACAAATCTCCGATGCTAAT |
| | GAATCTGCTACTGAAACTGCTTCTTCTAACAATGGTATGTCTGGTTGCGAA |
| | TCTTCCTTGAATAGATACAATGGTTTCGACGAGAACATCCAGAAGAACATT |
| | CATAACTTGGCCATGTACTTCAACAACCTGTCCATGAACTCTAAAGAGTC |
| | GTTCAACAACAACACTACTACTTTGACTTCTCCAACCTCTTCTGATACCTC |
| | CATTAACAACTTGGATACTGTTTCTACTGCTACCACCTCTGCTTCTATTTTC |
| | AGAGTTTACACCAACGGTATCAACCCAATCTTGGATAACAACAAGTTGGA |
| | ACAACAAACTTCTAACGGCACTGAAGTTTCTACCTTGTACAACCCATATAT |
| | CGATCCATCTAACCCACAGTTCAAGTCCAAAAAGTGGATCCAAAACATGG |
| | TCAACTTGATCAACAACGACCAGTCTTACTACAAGCCATATGAATTGGGTT |
| | GTTGCTGGACTGATTTGTGTGCTATGGGTTCTGATACAAACGATATTACTT |
| | ACCAGACCACCGTTTTTAACGCTCCATACAAATACGCTAGGTTGTTCTTCA |
| | ACCACTTGAACAGTAAGAGAAGATCCCAAGCCAAGAAATTCAAGGGTGTT |
| | ACTATCTTGCATAAGATGGATGGTTTGGTTGAATCTGGTGAGTTGTTGGTT |
| | GTTTTGGGTAGACCAGGTTCTGGTTGTACTACTTTATTGAAGTCTTTGACC |
| | GGTAACACCCATGGTTTCAAGATTTCTCAAGATTCCGAAATCACCTACAAC |
| | GGCATTTCTCAAAAGAAGATCAAAAAGAACTACAGGGGTGATGTTGTTTA |
| | CAACGCTGAAAACGATATCCATTTGCCACATTTGACTGTCTACCAAACTTT |
| | GTTGACTGTCGCTAGATTGAAAACCCCACAAAACAGATTCCACAACGTGT |
| | CTAGAGAACAATTCGCTGATCATATTACCCAAGTTGCTATGGCTACTTATG |
| | GTTTGTCTCATACCAGAAACACTAAGGTTGGTAACGATTTGGTTAGAGGT |
| | GTTTCAGGTGGTGAAAGAAAGAGAGTTTCTATTGCCGAAGTTTTCATCTG |
| | CGGTTCTAAGTTTCAATGTTGGGATAATGCTACCAGAGGTTTGGATGCTG |
| | CTACTGCTTTGGAATTTGTTAAGGCCTTGAAAACTCAAGCCTCTATCACTA |
| | ATGTTTCCGCTGCTGTTTCTATCTATCAATGCTCTAAAGATGCCTACGACT |
| | TGTTCGATAAGGTTTGTGTCTTGTACGAAGGTTACCAAATCTACTTCGGTA |
| | CTACTACCAACGCCAAAAAGTACTTCGAAAAGATGGGTTACTACTGCATC |
| | CAAAGACAAACTGTTGCTGATTTCATTACCGGTATCACCAATCCATCCGAA |
| | AGAATCATTAACCGTAACTTCATTAAGGCCAAGAAGTTCGTTCCACAGACT |
| | CCAAAAGAAATGAACGAATACTGGGAGAACAGCAAAGAGTACAAGCACTT |
| | GATTGAAGATATCGAAGAGTATAAGGTCAGGCAAAAGGCTAACGAAAACG |
| | AACAAATTGAGAAGATCAGAGAAGCCCATATTGCCAAGCAATCTAAAAAG |
| | GCTAGACCAGCTTCTCCATACACTGTTTCTTACTTCATGCAGGTCAAGTAC |
| | CTGTTGTTGAGGAATTTTTGGAGGATGAAGAACTCCTCCTCTATTACCTTG |
| | TTTCAAGTTTGTGGTAACACCGCCATGTCTTTGATTTTTGGTTCTATGTTCT |
| | ACAACGTCCTGAAGCCACCATCTACTACTCAATCTTTTTACTATAGAGGTG |
| | CCGCTATGTTCTTCGCTGTTTTGTTTAATGCTTTCAGCTCCTTGTTGGAAA |
| | TCTTCGCTATCTATGAAGCTAGGGAAATTACCGAAAAGCACAGAACCTAC |
| | TCACTGTATCATCCATCTGCTGATGCTTTGGCTTCAATCTTGTCTGAATTG |
| | CCACCAAAGATTATTACCTGCATCTGCTTCAACATCATCTACTATTTCATG |
| | GTGAACTTCAAGAGGAACGGTGGTAATTTCTTCTTCTACCTGTTGATTAAC |
| | TTCACCTCCGTTTTGGCTATGTCCCATTTGTTTAGAACTGTTGGTTCCATG |
| | ACCAAGTCTTTGTCTGAAGCTATGGTTCCAGCTTCCATTTTGTTGTTGGCT |
| | TTGTCTATGTACGTTGGTTTCGCCATTCCAAAGACTAAGTTGTTAGGTTGG |
| | TCTAAGTGGATCTGGTACATCAATCCATTGGCTTACATGTTCGAATCCTTG |
| | ATGGTTAACGAATTCCATAACACCAAGTTCGAATGCGCTACTTATATTCCA |
| | ACTGGTCCAGGTTACGAAAACATCTTGCCAGATCAAAGAGTTTGCTCTGT |
| | TGTTGGTAGTGTTCCAGGTCAAAATTACGTTTTGGGTGATGACTACTTGA |
| | GGGAATCTTACGACTATTACAACAAACATAAGTGGCGTGGTTTCGGTATT |
| | GGTTTGGCTTACGTTATTTTCTTCTTGGGCGTCTACTTGCTGTTTTGCGAA |
| | ATCAATGAAGGTGCTAAGCAAAAGGGTGAGATGTTGATTTTTCCACACGA |
| | TGTCTTGAAAAAGATGCACAAAGAAGGTCAAATCCAGGACTCTTCTTCATT |
| | GGCTATGGATTCCGATTTGGAAAAAGGTAATGGTAACGACTCATCCTTGG |
| | ATGTCAAAAACTCTAGCATCAACAACATTACCGACTCCATTTCTGGTAACA |
| | CTTTGACTGAAAAGCAGCAGTTGAAGGGTACGAATTTGACTTTGGAAGTT |
| | CAGCCAACTACCAACTCCTCTTCTAATTCTTCCGAAAAGGACATTGAAAAC |
| | AACGGCGTTATCTCCAAGTCCGAATCTATTTTTCATTGGAAGAACCTGTGC |
| | TACGACATCAATATTAAGGGTGAAAACAGGCGTATCTTGTCCAATGTTGAT |
| | GGTTGGGTTAAGCCAGGTACTTTAACTGCTTTGATGGGTGCTTCTGGTGC |
| | TGGTAAAACTACTTTGTTGGATTGCTTAGCTGAAAGAACCACTATGGGTAT |
| | AGTTACCGGTGATATGTTCGTTGACGGTAAATTGAGAGATGAATCTTTCC |
| | CAAGATCTATCGGTTACTGTCAACAGCAAGACTTGCATTTGAAAACCTCTA |
| | CCGTTAGAGAATCCCTGAGATTTTCTGCTTATTTGAGACAGCCATACTCC |
| | GTGTCCAGAAAAGAGAAAGAATTATACGTCGAAGAGGTCATCAAGATCCT |
| | GGAAATGGAAAAGTATGCTGAAGCAATAGTTGGTGTTCCTGGTGAAGGTT |
| | TGAATGTCGAACAAAGAAAAAGGTTGACCATCGGTGTTGAATTGGCTGCT |
| | AAGCCAAAGTTGTTGCTATTTTTGGATGAACCTACCTCCGGTTTGGATTCT |
| | CAAACTGCTTGGTCTATCTGTAAGCTGATGAGAAAGTTGGCTAATCATGG |
| | TCAAGCTATCTTGTTCACCATTCATCAACCATCCGCTATTCTGATGCAAGA |
| | ATTTGATAGGTTGCTGTTCCTACAGAAAGGTGGTAAGACTGTTTACTTTGG |
| | TGATTTGGGTAAGAGATGCCAAACCATGATTGATTACTTTGAAGCTAACG |
| | GTGCTGACAAGTGTCCAAAAGAGGCTAATCCAGCTGAATGGATGTTGGAT |
| | GTTGTCGGTGCTGCTCCAGGTTCTATTGCTAATCAAGATTACTACGAAGT |
| | CTGGCGTAATTCCCAAGAGTATAGAGATGTTCAAGAAGAGTTGAACAGGT |
| | TGGAAGAGGAATTTGCTGGTATTGAAAAACCAGTCGGTTCTGAAGAACAC |
| | AACGAATATGCTACACCTTTGCTATTCCAAATCAAGTACGTTGTCTTGAGG |
| | TTGTTCGATCAGTATTGGAGATCACCAACTTACTTGTGGTCCAAGTTTTTC |
| | TTGACCATCTACAACATGCTGTTCATCGGTTTCACTTTCTTCAAGGCTGAT |
| | TTGAGCTTGCAAGGTCTGCAAAATCAAATGCTGTCTTTGTTCATGTTCACC |
| | GTCATTTTCAACCCATTGATGCAACAATACTTGCCAATGTTTGTTCAACAG |
| | AGAGACTTATACGAAGCCAGAGAAAGACCATCTAGAACCTTTTCTTGGAT |
| | CACCTTTATCGTGTCCCAAATCTTGGTTGAAGTTCCCTGGAACTTTTTGTG |
| | TGGTACAATTGCCTACTTCATCTATTACTACTCTGTTGGCTTGTACCATAA |
| | TGCCTCTGTTGCAAATCAATTGCACGAAAGAGGTGCTTTGTTTTGGTTGTT |
| | CTCTTGTGCTTTCTTCGTGTTCATCTCCTCCATGTCCATTTTGGTTATCAG |
| | CTTTAACGAACATGATAGAAACGCTGCTAACTTGGGTTCTTTGATGTTCAC |
| | TATGTCTTTGGCTTTTTGTGGTGTAATGGCTGGTCCAGATATTTTTCCAAG |
| | ATTCTGGATCTTCATGTACAGAGTTTCTCCACTGACCTACTTTATCGATGG |
| | TTTGTTGTCTACAGGTTTGGCTAATGCTGATGTTACTTGTGCTGATTACGA |
| | ATTGGTCAGATTCTCTCCACCATCAGGTATGACTTGTGGTGAGTATATGC |
| | AACCCTATATTTCTATGGCTGGTACTGGTTACTTGACTGATACTGATGCTA |
| | CTGATACCTGTCATTTCTGCAATGTTTCTAAGACCAACGACTTCTTGAAGT |
| | CCGTTTCTTCTAAATACTCTAGAAGGTGGCGTAACTACGGTATCTTCTTGT |
| | GTTTCATCGTCTTTAACTTCGTTGCAGGTATTGGTCTATATTGGTTGGCTA |
| | GAGTTCCAAAACAATTCAGGATCGGTTTGTTTAAGAGGTTCAAGATCGAC |
| | TAA |
| SEQ ID NO: | MSLNSNSITNRSVVSMNPSEPISETVSQQISDANESATETASSNNGMSGCES |
| 1010 | SLNRYNGFDENIQKNIHNLAMYFNNLSMNSKESFNNNTTTLTSPTSSDTSINN |
| | LDTVSTATTSASIFRVYTNGINPILDNNKLEQQTSNGTEVSTLYNPYIDPSNPQ |
| | FKSKKWIQNMVNLINNDQSYYKPYELGCCWTDLCAMGSDTNDITYQTTVFN |
| | APYKYARLFFNHLNSKRRSQAKKFKGVTILHKMDGLVESGELLVVLGRPGSG |
| | CTTLLKSLTGNTHGFKISQDSEITYNGISQKKIKKNYRGDVVYNAENDIHLPHL |
| | TVYQTLLTVARLKTPQNRFHNVSREQFADHITQVAMATYGLSHTRNTKVGND |
| | LVRGVSGGERKRVSIAEVFICGSKFQCWDNATRGLDAATALEFVKALKTQAS |
| | ITNVSAAVSIYQCSKDAYDLFDKVCVLYEGYQIYFGTTTNAKKYFEKMGYYCI |
| | QRQTVADFITGITNPSERIINRNFIKAKKFVPQTPKEMNEYWENSKEYKHLIED |
| | IEEYKVRQKANENEQIEKIREAHIAKQSKKARPASPYTVSYFMQVKYLLLRNF |
| | WRMKNSSSITLFQVCGNTAMSLIFGSMFYNVLKPPSTTQSFYYRGAAMFFAV |
| | LFNAFSSLLEIFAIYEAREITEKHRTYSLYHPSADALASILSELPPKIITCICFNIIY |
| | YFMVNFKRNGGNFFFYLLINFTSVLAMSHLFRTVGSMTKSLSEAMVPASILLL |
| | ALSMYVGFAIPKTKLLGWSKWIWYINPLAYMFESLMVNEFHNTKFECATYIPT |
| | GPGYENILPDQRVCSVVGSVPGQNYVLGDDYLRESYDYYNKHKWRGFGIGL |
| | AYVIFFLGVYLLFCEINEGAKQKGEMLIFPHDVLKKMHKEGQIQDSSSLAMDS |
| | DLEKGNGNDSSLDVKNSSINNITDSISGNTLTEKQQLKGTNLTLEVQPTTNSS |
| | SNSSEKDIENNAVISKSESIFHWKNLCYDINIKGENRRILSNVDGWVKPGTLTA |
| | LMGASGAGKTTLLDCLAERTTMGIVTGDMFVDGKLRDESFPRSIGYCQQQD |
| | LHLKTSTVRESLRFSAYLRQPYSVSRKEKELYVEEVIKILEMEKYAEAIVGVPG |
| | EGLNVEQRKRLTIGVELAAKPKLLLFLDEPTSGLDSQTAWSICKLMRKLANHG |
| | QAILFTIHQPSAILMQEFDRLLFLQKGGKTVYFGDLGKRCQTMIDYFEANGAD |
| | KCPKEANPAEWMLDVVGAAPGSIANQDYYEVWRNSQEYRDVQEELNRLEE |
| | EFAGIEKPVGSEEHNEYATPLLFQIKYVVLRLFDQYWRSPTYLWSKFFLTIYN |
| | MLFIGFTFFKADLSLQGLQNQMLSLFMFTVIFNPLMQQYLPMFVQQRDLYEA |
| | RERPSRTFSWITFIVSQILVEVPWNFLCGTIAYFIYYYSVGLYHNASVANQLHE |
| | RGALFWLFSCAFFVFISSMSILVISFNEHDRNAANLGSLMFTMSLAFCGVMAG |
| | PDIFPRFWIFMYRVSPLTYFIDGLLSTGLANADVTCADYELVRFSPPSGMTCG |
| | EYMQPYISMAGTGYLTDTDATDTCHFCNVSKTNDFLKSVSSKYSRRWRNYG |
| | IFLCFIVFNFVAGIGLYWLARVPKQFRIGLFKRFKID- |
| SEQ ID NO: | ATGGTTGTCGGTTCCGTTGAACAACATCCAGGTGGTTATTCTGACGAAAATGCTTAT |
| 1011 | GGTGGTTTCGATGAAGCTATTAACGGTTCCGTAAAAGAATTGGCCAGATCCTTGATT |
| | GAAGAGAACAAGGATTCTACCAACTCCTTCAGTGAAAGAGAAAACACCGTTGAAAA |
| | CAACACCGGTATTAACCCAGTTGGTTTACAACCATCTGATGCTGAGTACAAACCAGA |
| | ATTGGATCCATCTTCTAACGACTTCTCATCTAAAGCTTGGATTGGTAACTTGGCTAA |
| | GGTTGTTGTTTCTGATCCAGATTACTACAAGCCATACACTGTTGGTTGTGGTTGGAG |
| | AAATTTGTCTGCTTTGGGTGCTTCTGCTGATGTTGCTTATCAAACTACTGTTGATAAC |
| | ATGCCCTGGAAGATTTTGTCTTGGTTGTATAGAATGGCTAGGCCAGCTAAAGAATCT |
| | GATACCTTCGAAATTTTGAAGCCAATGGATGGTTTGGTTAAGCCAGGTGAATTATTG |
| | GTTGTTTTGGGTAGACCAGGTTCAGGTTGTACTACTTTGTTGAAGTCCATTTCCTCTA |
| | ACACCCATGGTTTCAAGATTCCAAAGGATGCTACCATTTCTTACTCAGGTTTGTCCCC |
| | AAAGGATATCATCAATCATTTCAGAGGTGAAGTTGTCTACTGTCCAGAAGCTGATAT |
| | TCATTTGCCACATTTGACTGTGTTCCAAACCTTGTTGACTGTTGCTAGATTGAAAACC |
| | CCAAGAAACAGAATCAGAGGTGTTTCTAGAGAAGCTTGGGCTAGACATGTTACTGA |
| | AGTTACTATGGCTACTTACGGTTTGTCTCATACCAGAAATACCAAGGTTGGTAACGA |
| | ATTGGTTAGAGGTGTTAGTGGTGGTGAAAGAAAGAGAGTTTCTATTGCCGAAGTTA |
| | CCATTTGCGGTTCTAAGTTTCAATGTTGGGATAACGCTACTAGAGGTTTGGATGCTG |
| | CTACTGCTTTGGAATTTGTTAGAGCCTTGAAAACTCAAACCGAAATCGTTCATTCTG |
| | CTGGTTGTGTTGCTATCTACCAATGTTCTCAAGATGCCTACGATTTGTTCGATAAGG |
| | TTTGCGTTTTACACGGTGGTTACCAAATTTTCTTTGGTCCAGCTACTAAGGCCAAGTC |
| | TTACTTTGAAAGAATGGGTTACTACTGCCCATCTAGACAAACTACAGCTGATTTCTT |
| | GACTTCTGTTACTTCTCCAGCCGAAAGAATCATCAACAGAGAATTCACTGAAAAGG |
| | GTATTGCTGTTCCACAAACTGCTGAAGAAATGTCCGATTATTGGAGAAACTCCCCTG |
| | AATACCAAGAATTGATTGCTGAAGTTGACGAAACCATCTCTCAAGATCACGAAAAG |
| | TCCTTGGAAGTCATCCAAGAATCTCATAACGCTAGACAATCTAAGAGAGCTAGAAG |
| | GGCTGAACCATATACTGTTTCTTACTTCATGCAGGTCAAGTACCTGATGATTAGAAA |
| | CTTCTGGCGTATCATCAACTCCTCTTCTATTACCGTTTTCCAGATCATCGGTAACTCT |
| | GTTATGGCTTTGTTGTTGGGTTCTATGTTCTACAAGGTCCTGAAGAAAAGTTCTACC |
| | GGTACTTTTTACTATAGAGGTGCTGCTATGTTCTTCGCCATTTTGTTTAATGCCTTCA |
| | GCTCCTTGTTGGAGATCTTCTCATTATATGAAGCTAGACCAGTTACCGAAAAGCACA |
| | AGACTTACTCATTATACAGACCTTCCGCTGATGCTTTTGCTTCTGTTTTGTCTGAAAT |
| | TCCAGCTAAGGTTTTGACCGCTGTTTGTTTCAATATTGCCTTCTACTTCTTGGTGAAC |
| | TTCAGAAGAGATGCTGGTAGATTCTTCTTCTACTTTCTGATCAACATTATCGCCGTGT |
| | TCGTTATGTCTCATATGTACAGATGTGTTGGCTCTTTGACTAACACTTTGACTGAAGC |
| | TATGGTTCCAGCCTCTATTTTGTTGTTAGCTATGGCAATGTACACCGGTTTTGCTATT |
| | CCAAAGACTAAGATGTTAGGTTGGTCTAAGTGGATTTGGTGGATTAACCCATTGTCC |
| | TACTTGTTCGAATCTTTGATGGGTAATGAATTCCACGACCAAAAGTTCCCATGTACC |
| | ACTTTTGTTCCAAGAGGTGGTGATTACGATCATGTTACAGGTACTGAAAGAGTTTGC |
| | TCTGTTGTTGGTTCAAAAGCTGGTCAAGATTACGTTTTGGGTGACGACTACCTGAAA |
| | GAATCTTATGGCTATTTGATCAAGCACAAGTGGCGTGGTTTTGGTGTTGGTATGGCT |
| | TACTTGATCTTCTTTTTCTTCCTGTACTTGTTCTTGTGCGAAGTTAATGAAGGTGCTA |
| | AGCAAAAGGGTGAGATTTTGGTTTTTCCAAGGTCCATTGTCAGAAAGATGAGAAAG |
| | CAGAACAAGCTGAAAGAAGAAGATAGGGATCCAGAAGATGTTGAAAAGATTGCTG |
| | GTTCTTCTGGTTCTACCGATAAGATGTTGTTGAAGGACTCCTCCGAATCCATTGATG |
| | AAGAAAATGAACCATCTGCCTTAGGTGGTTCTCAAGCTATTTTTCATTGGAGGAACT |
| | TGTGCTACGAAGTCCAAATCAAAGGTGACACTAGAAGGATCTTGAACAATGTTGAT |
| | GGTTGGGTAAAACCTGGTACTTTGACAGCTTTAATGGGTGCATCTGGTGCTGGTAA |
| | AACTACTTTATTGGATTGCTTGGCTGAAAGGGTTACAATGGGTGTCATTACTGGTGA |
| | TATTTTCGTGAACGGTAAGATCAGGGATGAATCTTTCCCAAGATCTATTGGTTACTG |
| | TCAACAACAAGACCTGCATTTGAAAACTGCTACCGTTAGAGAATCGTTGATTTTCTC |
| | AGCTATGTTGAGACAGCCAAAGTCTGTTCCAGTTTCGGAAAAGAAAAAGTATGTTG |
| | ACGACGTCATCCGTATCTTGGAAATGGAACAATATGCTGATGCAGTTGTTGGTGTTC |
| | CAGGTGAAGGTTTGAATGTTGAACAGAGAAAAAGATTGACCATCGGTGTTGAATTG |
| | GTTGCTAAACCTAAGCTGTTGGTCTTTTTGGATGAACCTACTTCCGGTTTAGATTCTC |
| | AAACTGCTTGGTCTATTTGCCAGCTAATGAAGAAATTGTCTAAGCACGGTCAAGCTA |
| | TCTTGTGCACTATTCATCAACCATCCGCTATGCTAATGCAAGAATTTGATCGTTTGCT |
| | GTTCTTGCAGAAAGGTGGTAATACTGTTTACTTCGGTGATTTGGGTAAAGACTGCA |
| | AGACCATGATTGACTACTTTGAATCTAATGGTGCTGATCCATGTCCACCTGATGCTA |
| | ATCCAGCTGAATGGATGTTGGAAGTTGTCGGTGCTGCTCCAGGTACTCATGCTAAT |
| | AGAGATTACCATGAAGCTTGGAGGAATTCTCCAGAATATCAAGCAGTTCAACAAGA |
| | GTTGGACAGGTTGGAAAATGAGTTGCAATCCTTGGATGAAGAGGATGGTGTTGAA |
| | AAACATAAGTCTTTCGCTACCGATGTGTTCACCCAGATTAAGTTTGTTTCCTTCAGAT |
| | TGCAACAACAGTATTGGAGATCACCACAGTATTTGTGGTCCAAGTTCTTCTTGACTG |
| | TTATCTCCGAGTTGTTCATCGGTTTCACTTTCTTTAAGGCTGACAGATCCATGCAAGG |
| | CTTGCAAAATCAAATGTTGGCTGTTTTCATGTTCACCGTTATCTTTAACGCTATCTTG |
| | GAGCAATACTTGCCAAACTATGTAGAACAAAGGGACTTGTACGAAGCTAGAGAAA |
| | GACCATCTAGAACCTTTTCATGGTTCGCTTTCATCGTGTCCCAAATTTTGGTTGAAGC |
| | TCCATGGAATTTCTTGGCTGGTACTATTGCTTACTTTATCTACTACTACCCAATCGGC |
| | TTTTACCAAAATGCTTCAGCTGCTGGTCAATTGCACGAAAGAGGTGCTTTGTTTTTCT |
| | TGTGGTCTACCGCTTTTTATGTTTGGGTTGGTTCTATGGCTTTACTGGCCAATAGCTT |
| | TATTGAACATGATGTTTCTGCTGCCAATTTGGCCAACTTGTGTTTTACTTTGGCTTTG |
| | TCTTTTTGCGGTGTTATGACTACTCCAGATGCTATGCCTCATTTCTGGATTTTCATGT |
| | ACAGAGTCTCTCCCTTGACCTACTTCATTGATGCTGTTTTGGCAGTTGGTATTGCCAA |
| | CGTTGATATTGAATGCTCCAACTACGAATTTGTCCAATTCACTCCACCACAAGGTAG |
| | AACATGTGGTGAGTATATGCAAGCCTACTTGAAATCTGCTGGTACAGGTTATTTGAA |
| | GGATGCAAATGCTACAGCTCAATGTTTGTTGTGTCCTTTGTCCAGAACTAACGACTA |
| | CTTGTCTCAAGTTAACTCCCATTATTCTCACAGATGGCGTAACTACGGTATTTTCATC |
| | TGTTACATCGTGTTCAACTATGTTGCTGCCGTTTTCTTGTATTGGTTGGCTAGAGTTC |
| | CAAAGAAGGACACCTTTTTCAGCAAGATCTTCAGTAAGAAGTAA |
| SEQ ID NO: | MVVGSVEQHPGGYSDENAYGGFDEAINGSVKELARSLIEENKDSTNSFSERENTVENN |
| 1012 | TGINPVGLQPSDAEYKPELDPSSNDFSSKAWIGNLAKVVVSDPDYYKPYTVGCGWRNL |
| | SALGASADVAYQTTVDNMPWKILSWLYRMARPAKESDTFEILKPMDGLVKPGELLVVL |
| | GRPGSGCTTLLKSISSNTHGFKIPKDATISYSGLSPKDIINHFRGEVVYCPEADIHLPHLTVF |
| | QTLLTVARLKTPRNRIRGVSREAWARHVTEVTMATYGLSHTRNTKVGNELVRGVSGGE |
| | RKRVSIAEVTICGSKFQCWDNATRGLDAATALEFVRALKTQTEIVHSAGCVAIYQCSQD |
| | AYDLFDKVCVLHGGYQIFFGPATKAKSYFERMGYYCPSRQTTADFLTSVTSPAERIINRE |
| | FTEKGIAVPQTAEEMSDYWRNSPEYQELIAEVDETISQDHEKSLEVIQESHNARQSKRA |
| | RRAEPYTVSYFMQVKYLMIRNFWRIINSSSITVFQIIGNSVMALLLGSMFYKVLKKSSTG |
| | TFYYRGAAMFFAILFNAFSSLLEIFSLYEARPVTEKHKTYSLYRPSADAFASVLSEIPAKVLT |
| | AVCFNIAFYFLVNFRRDAGRFFFYFLINIIAVFVMSHMYRCVGSLTNTLTEAMVPASILLL |
| | AMAMYTGFAIPKTKMLGWSKWIWWINPLSYLFESLMGNEFHDQKFPCTTFVPRGGD |
| | YDHVTGTERVCSVVGSKAGQDYVLGDDYLKESYGYLIKHKWRGFGVGMAYLIFFFFLYL |
| | FLCEVNEGAKQKGEILVFPRSIVRKMRKQNKLKEEDRDPEDVEKIAGSSGSTDKMLLKD |
| | SSESIDEENEPSALGGSQAIFHWRNLCYEVQIKGDTRRILNNVDGWVKPGTLTALMGA |
| | SGAGKTTLLDCLAERVTMGVITGDIFVNGKIRDESFPRSIGYCQQQDLHLKTATVRESLIF |
| | SAMLRQPKSVPVSEKKKYVDDVIRILEMEQYADAVVGVPGEGLNVEQRKRLTIGVELVA |
| | KPKLLVFLDEPTSGLDSQTAWSICQLMKKLSKHGQAILCTIHQPSAMLMQEFDRLLFLQ |
| | KGGNTVYFGDLGKDCKTMIDYFESNGADPCPPDANPAEWMLEVVGAAPGTHANRDY |
| | HEAWRNSPEYQAVQQELDRLENELQSLDEEDGVEKHKSFATDVFTQIKFVSFRLQQQY |
| | WRSPQYLWSKFFLTVISELFIGFTFFKADRSMQGLQNQMLAVFMFTVIFNAILEQYLPN |
| | YVEQRDLYEARERPSRTFSWFAFIVSQILVEAPWNFLAGTIAYFIYYYPIGFYQNASAAG |
| | QLHERGALFFLWSTAFYVWVGSMALLANSFIEHDVSAANLANLCFTLALSFCGVMTTP |
| | DAMPHFWIFMYRVSPLTYFIDAVLAVGIANVDIECSNYEFVQFTPPQGRTCGEYMQAY |
| | LKSAGTGYLKDANATAQCLLCPLSRTNDYLSQVNSHYSHRWRNYGIFICYIVFNYVAAVF |
| | LYWLARVPKKDTFFSKIFSKK |
| SEQ ID NO: | ATGTCCGAGTCCTCCATTCAAAACTTCACTACTGATTTCGCTAACGAAGAG |
| 1013 | GATTCTTACAATGGTGATCCAGATGTTTCCTCCGTTAGAGATTTGGCTAGA |
| | TCTTTCACCAATATCAGCCTGAACTCTTCCTCCTTGTATGATATTCCATTG |
| | AAGAACGAGGATGGCAACTTGGATATTGAGGATTCTAAGTACAACTCCAA |
| | GTTGGACCCAAATTCTCCAGATTTTAATGCCCATTTCTGGATGAAGAACCT |
| | GCACAAGATTAGAAATACCGATCCAGACTACTACAAGTCCACTAATTTGG |
| | GTTTAGTCTACAAGAACTTGTCCTGCGTTGGTGAATCTTCTGATGTTATGT |
| | ACCAAACCACCTTCATCTCCATCTTGCAATATTTTGCTGAAACGCTGTACA |
| | AGAAGTTGAGGCCATCTAAACCAGAAGATATGTTCACCATTTTGAAGCCA |
| | ATGGACGGTATTTTGAAACCAGGTTCTTTGAACGTTGTTTTGGGTAAACCT |
| | GGTTCTGGTTGTACTACTCTGTTGAAAACTATTGCTGCTTCTACCTACGGT |
| | TTCGAAGTTGCTAAAGATTCCTTCATGTCTTACGATGGTTTGGCTCCAAAG |
| | GATATTAACAAACACTACAGAGGTGATGTTGTCTACCAAGCTGAAACCGA |
| | TATTCATTTCCCAAACTTGACTGTGTTCGAAACCTTGAAATCTGTTGCTTT |
| | GTTGACTACTCCACGTAACAGAATCAAAGGTTTGACCAGAGATCAATTCG |
| | CTACTCATATGGCTGAAGCTACTATGGCTATGTATGGTTTGTCTCATACCA |
| | GAAACACTAAGGTCGGTAACGAATTCATTAGAGGTGTTTCAGGTGGTGAG |
| | AGAAAAAGAGTTTCCATCTGCGAAATCTCATTGATCAACGGTAAGATCGTT |
| | TGCTACGACAATTCTTCTAGAGGTTTGGATTCTGCCTCTACCTTGTCTTTC |
| | ATTAAGTGTTTGAAAACCGCCTCCATTGCTAATGATACAACTGCTGTTGTT |
| | GCTATCTACCAATGTTCTCAAGAAGCCTACGATTTGTTCGATAACGTCATC |
| | GTTTTGGATCATGGTTACCAGTTGTATAATGGTCCAGCTCAATTGGCTAAG |
| | CAATACTTTTTGGATATGGGTTATGTTTGCCCAGACAGACAAACTACTGCT |
| | GATTTCTTGACTGCTATCACCTCTCCAAAAGAAAGGATCCAGAACAAAGA |
| | AATGCTGTCCAGAGGTATTAAGATCCCATCTACTCCAGAGGAAATGTACA |
| | ACCATTTCAAGAAGTCCCAAATCTACCAGGACTTGTTGAAGCAAATCGAT |
| | GACTACAACTCTAACATCAACGAAGAGACAAAAGAGAAGTTCATTGCTTCT |
| | CATGCTGCTGCTCAATCTAAAAGAGCTAAACCATCTTCCAGCTACAGATT |
| | GTCTTACGGCTTGCAAATCAAATACTTGTTGCAGAGAAACTTCACCAGGA |
| | TCAAGAACGATATTGGCTTGTCTGTTTTCATCGTCTTGGCCAATTCTTTGA |
| | TGGCTTTGGTTATTGCCTCTATGTTCTACAAGGTCATGTACCATACTGACA |
| | CCTCCACATTTTTCTTTCGTGGTGGTTCTTTGTTTTACGCTATCCTGTTTAA |
| | CTCCTTCAGCTCCTTGTTGGAAGTTATGACATTATACGAAGCCAGGTCCA |
| | TTATCGAGAAGCAAAAGAATTTGGCAATGTACCATCCATCTGCAGAAGCT |
| | ATTGCTTCCATTTTGTCTCAATTGCCCTCTAAGTTGTTGACCAACATCTGT |
| | TTCAACCTGCTGTTTTACTTCCTGGCTAACTTGAGAAGAGAACCAGGACC |
| | ATTCTTTTTCTACCTGTTGTTGAACTTCACCTGTGTCTTGACTATGTCTCAC |
| | TTGTTCAGATTCATTGGTTCTGCTACTAAGTCTTTTCCAGAAGCCATGGTT |
| | CCAGGTTCAGTTGTTTTGTTGGCTTTGACAATGTATGCTGGTTTCGCTATT |
| | CCAAAGACCAAAATGTTAGGTTGGTCTAAGTGGATCTACTGGATTAACCC |
| | ATTGCAGTACGGTTTTGAGTCCTTGATGATCAACGAATTCCACGACAGAA |
| | ACTTCGAATGCTCTCAATATGTTCCAACTGGTGGTGATTACAACTCTGTTT |
| | CTTTGGATTACAAGACCTGTTCAGCTGTTGGTGCTGTTCCAGGTGAAGAT |
| | TTTGTTAACGGTGATCAGTTCCTGAAGTTGTCATATGGTTATTCCCATGGT |
| | CATAAGTGGCGTGGTTTTGGTGTTTTGGTTGGTTTTGCTATCTTCTTCTTC |
| | TCGATCTACTTGTTCTTCACCGAATTCAACGAATCCGCTAAACAAAAGGGT |
| | GAGATTATCTTGTTCCCACAGTCTATCGTTAGGAAGATCAAAAAGCAAAAC |
| | AAGTACATGCAGTCCCATCCAGAAGATTTGGAAAACCCAATTGATTCCAA |
| | GGATAGAGCCAACGAAAAGTCCATCATTGATAACGATGACTCCAGATCTT |
| | CTGCCTACAAATCTAAGGATGAATCCTCCTTGGACTCTGATAACGTTGGTT |
| | TGTCAGAATCAGAAGCTACATTGCATTGGAGAGATTTGTGTTACGACATC |
| | AAGATTAAGGGTGAAACCAGACGTATCTTGAACAAAGTTGATGGTTGGGT |
| | TGCCAAGAGATCTATTACTGCTTTGATGGGTTCTTCTGGTGCTGGTAAAA |
| | CTACTTTGTTGGATTGCTTGGCTTCTAGAGTTACTATGGGTGTTGTTACTG |
| | GTAAGATCTTGGTCAATGGTAAGCAAAGAGATAACAGCTTCCCAAGATCT |
| | ATTGGTTACTGTCAACAACAAGACTTGCATTTGTCTACTGCCACTGTTAGA |
| | GAATCCTTGAGATTTTCTGCTTACTTGAGACAATCCGCTGCCATCTCTAAG |
| | AAAGAAAAGGACGAATACGTTGAGAACGTCATCAAAATCTTGGACATGGA |
| | AAAGTACGCTCATGCAGTTGTTGGTGTTATGGGTGAAGGTTTAAATGTCG |
| | AACAACGTAAGAGATTGACCATCGGTGTTGAATTGGCTGCTAAACCTAAG |
| | CTGTTGATGTTTTTGGATGAACCTACATCTGGTTTGGACTCTCAAACTGCT |
| | TGGTCTATTTGTCAGTTGATGAGAAAGTTGGCCAATCAAGGTCAAGCTATT |
| | TTGTGCACTATTCATCAACCATCCGCCATCTTGATTCAAGAATTTGATAGG |
| | TTGTTGTTCTTGCAGCCAGGTGGTAAGACTGCTTATTTTGGTGACTTAGGT |
| | GATGGTTGTAAGACCATGATTAAGTACTTCGAATCTAAGGGTGCTGAAAA |
| | GTGTCCACCAGATGCTAATCCAGCTGAATGGATGTTGGATATAGTTCAAG |
| | CTAGAGATTACCACGAAGCTTGGAGACAGTCTGATGAATTCAAAGAAGTT |
| | AAGTCCACCTTGGAAGAGATGGAAAGAGAATTGCCAAAGATCCAAATGAC |
| | CGAGGATAAGTATACTCATGCTTCATTCGCTGCATCTTTTTGGTTGCAGTA |
| | CAAGTTGGTTTTCATCAGGGTCATGCAACAAAATTGGAGAACTCCATTTTA |
| | CCTGTGGTCCAAGTTTTTCTTGGTCGTCTACTCCGAAATTTTCATCGGCTT |
| | TACTTTCTTCAAGGCCGATCATACCTTACAAGGTCTGCAAAATCAAATGTT |
| | GGCCGTTTTCATGTTCACCGTCTTGTTTAATCCATACTTGCAACAATACCT |
| | GCCAGTGTTTAAGCAACAAAGAGACTTGTACGAAGCAAGAGAAAGACCAT |
| | CTAGAACCTTTTCTTGGGTTGCTTTCATTACTGCTCAAATGGCTGCTGAAA |
| | TTCCATCTAATTTTGCCTCTGGTTGTTTGGCTTTCTTCTGTTACTTTTACCC |
| | AATCGGTTTCTACCAAAACGCTTCCAATTCTGGTCAATTGTCTGAAAGATC |
| | CGGTCTGTTCTTCTTGTATTCTATTGCCTTCTTCATCTACACCGGTTCTTTC |
| | GCTGTTTTAGTTGCTTCTCCATTTGATGATCCACAAGCTGGTGGTCATATT |
| | TCCTCCATTATTTTCACTATGGCCTTGGCTTTTAACGGTGTTTTTGTCGGT |
| | CCAAACGAAATGCCAGGTTTTTGGAAGTTTATGTACAGGGTTTCTCCAAT |
| | GACCTACTTGGTTGACGGTTTGTTGTCTGTTGGTATTGCTAACAACAACG |
| | CTGAATGCTCTACTTACGAATTCAGATCTATAGTGCCACCAGAAAACATGA |
| | CTTGTGGTGAATATTTGGACCCATACTTGAAAGCTGCTGGTACTGGTTATT |
| | TGTTGGACTCAGGTAATGCTGAAGTCTGTAAGTTGTGTTCCGTTTCTTCTA |
| | CCAACGCTTTCTTGTCATCTGTCTCTTCAAAGTATTCTAGAAGGTGGCGTA |
| | ACTTCGGTATTTTCTTGGCTTACATCGTTTTCGATTACGCCTGTACCATTTT |
| | CGTTTATTGGTTGGCTAGAGTCCCAAAGAAATCCTCTAGAGTCAAAGAAC |
| | AATCCGATGCTTCCGTCGAAAATTCCAAGTCTGATGTTGAATCGAAGAAG |
| | AACTAA |
| SEQ ID NO: | MSESSIQNFTTDFANEEDSYNGDPDVSSVRDLARSFTNISLNSSSLYDIPLKN |
| 1014 | EDGNLDIEDSKYNSKLDPNSPDFNAHFWMKNLHKIRNTDPDYYKSTNLGLVY |
| | KNLSCVGESSDVMYQTTFISILQYFAETLYKKLRPSKPEDMFTILKPMDGILKP |
| | GSLNVVLGKPGSGCTTLLKTIAASTYGFEVAKDSFMSYDGLAPKDINKHYRG |
| | DVVYQAETDIHFPNLTVFETLKSVALLTTPRNRIKGLTRDQFATHMAEATMAM |
| | YGLSHTRNTKVGNEFIRGVSGGERKRVSICEISLINGKIVCYDNSSRGLDSAS |
| | TLSFIKCLKTASIANDTTAVVAIYQCSQEAYDLFDNVIVLDHGYQLYNGPAQLA |
| | KQYFLDMGYVCPDRQTTADFLTAITSPKERIQNKEMLSRGIKIPSTPEEMYNH |
| | FKKSQIYQDLLKQIDDYNSNINEETKEKFIASHAAAQSKRAKPSSSYRLSYGL |
| | QIKYLLQRNFTRIKNDIGLSVFIVLANSLMALVIASMFYKVMYHTDTSTFFFRG |
| | GSLFYAILFNSFSSLLEVMTLYEARSIIEKQKNLAMYHPSAEAIASILSQLPSKL |
| | LTNICFNLLFYFLANLRREPGPFFFYLLLNFTCVLTMSHLFRFIGSATKSFPEA |
| | MVPGSVVLLALTMYAGFAIPKTKMLGWSKWIYWINPLQYGFESLMINEFHDR |
| | NFECSQYVPTGGDYNSVSLDYKTCSAVGAVPGEDFVNGDQFLKLSYGYSH |
| | GHKWRGFGVLVGFAIFFFSIYLFFTEFNESAKQKGEIILFPQSIVRKIKKQNKY |
| | MQSHPEDLENPIDSKDRANEKSIIDNDDSRSSAYKSKDESSLDSDNVGLSES |
| | EATLHWRDLCYDIKIKGETRRILNKVDGWVAKRSITALMGSSGAGKTTLLDCL |
| | ASRVTMGVVTGKILVNGKQRDNSFPRSIGYCQQQDLHLSTATVRESLRFSAY |
| | LRQSAAISKKEKDEYVENVIKILDMEKYAHAVVGVMGEGLNVEQRKRLTIGVE |
| | LAAKPKLLMFLDEPTSGLDSQTAWSICQLMRKLANQGQAILCTIHQPSAILIQE |
| | FDRLLFLQPGGKTAYFGDLGDGCKTMIKYFESKGAEKCPPDANPAEWMLDIV |
| | QARDYHEAWRQSDEFKEVKSTLEEMERELPKIQMTEDKYTHASFAASFWLQ |
| | YKLVFIRVMQQNWRTPFYLWSKFFLVVYSEIFIGFTFFKADHTLQGLQNQMLA |
| | VFMFTVLFNPYLQQYLPVFKQQRDLYEARERPSRTFSWVAFITAQMAAEIPS |
| | NFASGCLAFFCYFYPIGFYQNASNSGQLSERSGLFFLYSIAFFIYTGSFAVLVA |
| | SPFDDPQAGGHISSIIFTMALAFNGVFVGPNEMPGFWKFMYRVSPMTYLVD |
| | GLLSVGIANNNAECSTYEFRSIVPPENMTCGEYLDPYLKAAGTGYLLDSGNA |
| | EVCKLCSVSSTNAFLSSVSSKYSRRWRNFGIFLAYIVFDYACTIFVYWLARVP |
| | KKSSRVKEQSDASVENSKSDVESKKN |
| SEQ ID NO: | ATGTCCTCCTCTACCAACTCCGATGATGAATCTGGTAGATTGCATACTTAC |
| 1015 | ACCGGTATTGATGCTCAAGCTGAAGCTACTATTAAGAACTTGGCTAGAAC |
| | TTTGACCGCTCAGTCTTTGAACTCTATCAACTCTTCTAACAACAACAACGA |
| | GGATTCCACCTCTGTTAACGATCATCAATCCATCTTCTCTAACTTGGAAGG |
| | TATCAACCCAGTTTTGACTAACCCAGAACAATCTGGTTACGACGAAAAATT |
| | GGATCCTACCTCTGATTACTTCTCTTCTACTGCTTGGGTTAAGAATATGGC |
| | CAGATTGGCTAAATCTGATCCAGATTACTACAAGCCATACTCTTTGGGTTG |
| | TGTTTGGAAAGATTTGACTGCTTCTGGTACTTCCTCTGATGTTGAATATCA |
| | AGCTACTGCTTTGAACGCTCCATTGAAAGCTTTGTCTTACTTGTACAGAGA |
| | AGTCAAGCCAGTCAACGAAGAAAACTCCTTCCAAATCTTGAAAGAGATGG |
| | AAGGTTGTATTAACCCAGGTGAGTTGTTGGTTGTTTTGGGTAGACCAGGT |
| | TCTGGTTGTACTACTTTGTTGAAAACCATCTCCACTAACACCCATGGTTTC |
| | CATGTTGGTAAGAACGCTACTATTTCTTACTCTGGTTTCACCCCAAAAGAG |
| | ATCAAAAAGCACTATAGAGGTGAAGTTGTTTACAACGCCGAATCCGATATT |
| | CATTTGCCACATTTGACTGTCTACCAAACCTTGTACACTGTCTCTAGATTG |
| | AAAACTCCACAGAACAGAATCAAGGGTGTTGACAGAGATACTTTCGCTAG |
| | ACATATTACCGAAGTTGCTATGGCTACTTATGGTTTGTCTCATACCAGAGA |
| | TACCAAGGTTGGTAACGATTTGGTTAGAGGTGTTTCTGGTGGTGAAAGAA |
| | AGAGAGTTTCTATTGCCGAAGTCTCTATTTGCGGTTCTAAGTTTCAATGTT |
| | GGGATAATGCTACCAGAGGTTTGGATTCTGCTACAGCTTTGGAATTCATT |
| | AGAGCCTTGAAAACTGATGCTACCACCGTTAATTCTTGTTCTACTGTTGCT |
| | ATCTACCAATGCTCTCAAGATGCTTACGATTTGTTCGATAAGGTTTGCGTT |
| | TTGGACCAAGGTTACCAAATCTATTTTGGTCCAGGTACTAAGGCTAAGGC |
| | TTACTTTGAAAAGATGGGTTACGTTTCCCCAGATAGACAAACTACTGCTGA |
| | TTTCTTGACTGCTGTTACTTCTCCAGCTGAAAGGATCTTGAATCAAGAGTA |
| | CTTGAAGAAGGGTATCTCCATTCCACAAACTCCAGAAGATATGAACACTTA |
| | CTGGAAGAACTCCCAAGATTACAAGGATTTGATGGCCGAAATTGACGAGA |
| | AGTTGAACAACAATGTCGAGGACTCTAGAGAATTGGTCAAAGAAGCTCAT |
| | ATTGCCAAGCAGTCTAATAGATCTAGACCATCTTCACCATACACCGTCAAT |
| | TATTTCTTGCAGATCAAGTACCTGCTGACCAGAAATGTTTGGAGAATCAAA |
| | AACAACCCCTCCGTCAATTTGTTCATGGTTTTTGGTAATTCCTCCATGGCC |
| | TTGATTTTGGGTTCTATGTTTTACAAGGTTATGCTGCACGATTCTACCTCT |
| | ACTTTTTACTTTAGAGGTTCCGCTATGTTCTTCGCCATTTTGTTTAATGCTT |
| | TCTCGTGCTTGTTGGAGATCTTCACATTATATGAAGCCAGACCAATTACCG |
| | AAAAGCACAAGACTTACAGCTTGTATCATCCATCTGCTGATGCTTTCGCCT |
| | CCATTATTTCTGAATTGCCAACAAAAGCTGCTATCGCTGTTTGCTTCAACA |
| | TTATCTTCTATTTCTTGGTCAACTTCAGAAGAAACGGCGGCGATTTCTTTT |
| | TCTACTTGCTGATTAACATCGTTGCCGTTTTCGCTATGTCTCATATGTTTA |
| | GAACTGTTGGTGCTGCTACTAGAACATTGTCTGAAGCTATGTTTCCAGCC |
| | TCCATGTTGTTATTGGCTATGTCTATGTATACCGGTTTCGCTATTCCAAAG |
| | ACCAAAATGTTAGGTTGGTCTGAATGGATTTGGTACTTGAATCCATTGGC |
| | CTACTTGTTCGAATCCTTGATGATTAACGAATTCCACAACAGAGATTTCCC |
| | ATGCGCTCAATATGTTCCACAAGGTCCAGCTTATGTTAATGCTACTGGTAC |
| | TGAAAGAGTCTGCATTTCTTTGGGTGCTGTTCCAGGTGAAGATTTTGTTAA |
| | TGGTGATGCTTACATTAAGGCCAACTATGGTTATGAACATAAGCACAAATG |
| | GCGTGGTTTTGGTGTTGGTATGGCTTTTGCTGTTTTCTTCTTGGGTGCTTA |
| | TTTGGCTGTTTGCGAATTCAATGAAGGTGCTAAACAAAAGGGTGAGATCT |
| | TGGTTTTCCCAAAGTCCATTATCCGTAAGATGAAGAAGAATGGTCAATTGC |
| | CACAAAGAGCAAGACCAGATAAGAGAGAAGAAGATATCGAAAAGGGTACT |
| | ACCGATGAATCTTCCGTTAACGATAGAAAGTTGTTGGAGGACTCCGAAAT |
| | TTCTTCCGACGAAAACAAAGAAGAGTCCATCGGTTTGTCTAAGTCTACCG |
| | CTATTTTTCATTGGAGGAACTTGTGTTACGACGTCCAAATCAAAGACGAAA |
| | CGAGAAGAATCTTGTCCGATGTTGATGGTTGGGTAAAACCAGGTACTTTG |
| | ACAGCTTTGATGGGTGCTTCAGGTGCTGGTAAAACTACATTATTGAACTG |
| | TTTGGCTGAAAGGGTTACCATGGGTACTATTACTGGTGATGTTTTCGTTGA |
| | CGGTAGATTGAGAGATGAATCCTTTCCAAGATCTATCGGTTACTGTCAAC |
| | AACAAGACTTGCATTTGAGAACCTCTACCGTTAGAGAATCTTTGAGATTCT |
| | CTGCCTATTTGAGACAACCATCTGATGTTTCTACCGAAGAAAAGAACGCTT |
| | ACGTCGAAGAAATCATCACTATCTTGGAGATGGATAACTACGCTGATGCT |
| | ATAGTTGGTGTACCTGGTGAAGGTTTGAACGTTGAACAAAGAAAAAGATT |
| | GACCATCGGTGTTGAATTGGCTGCTAAACCTAAGCTATTGGTTTTCTTGG |
| | ATGAACCTACATCTGGTTTGGATAGTCAAACAGCTTGGGCTATTTGTCAGT |
| | TGATGAGAAAATTGGCTTCTCATGGTCAAGCTATTTTGTGCACTATTCATC |
| | AACCATCCGCTATCTTGATGCAAGAATTTGATAGGTTGTTGTTCTTGCAAA |
| | GAGGTGGTAAGACTGTTTACTTCGGTGAATTAGGTCATGGTTGCCAAAAG |
| | ATGATCGACTACTTTGAATCTCATGGTTCTCATAAGTGTCCACCAGAGGCT |
| | AATCCAGCCGAATGGATGTTGGAAGTTGTAGGTGCTGCTCCTGGTTCACA |
| | TGCTAATCAAGATTATTACGAAGTCTGGCGTAACTCCGAACATTTTAAAGC |
| | TGTTCATGAAGAGTTGGACAGGATGGAAAAGGATTTGCCAGGTCAAGCAA |
| | AAGAAGAGGACGATCCAACTGCTCACAAAGAATTTGCTACAGGTATTCCA |
| | TACCAGATCAACTTGGTTTCTGAAAGGTTGTTCCAACAATATTGGAGATCC |
| | CCACAATACTTGTGGTCCAAGTTTATCTTGACGATCTTCAACATGCTGTTC |
| | ATCGGTTTCACTTTCTTCAAGGCTAACACTAGCTTGCAAGGTCTACAAAAC |
| | GAAATGTTGGCCATTTTCATGTTCACCGTTATCTTCAACCCACTGTTGCAG |
| | CAATACTTGCCAAATTTTGTCGAACAGAGAGACTTGTACGAAGCTAGAGA |
| | AAGACCATCCAGAACATTCTCATGGAAGGCTTTTATCGTTTCCCAGATTTT |
| | GGTTGAGGTCTTTTGGAATTTCTTGGCTGGTTCTTTGGCTTACTTCATCTA |
| | CTATTACGCCATTGGTTTTTACCACAATGCATCTGTTGCTGGTCAATTACA |
| | CGAAAGAGGTGCTTTGTTTTGGTTGTTCTCAACTGCCTTTTTCGTTTACTG |
| | TGGTTCTATGGGTACAATGGCCATCTCCTTTATTGAAATTGCTGAAAACGC |
| | TGCCAACATGGCTTCATTGATGTTTACTATGTGTTTGTCCTTCTGCGGTGT |
| | TATGGTTACTAAGGATGCTATGCCAAGATTCTGGATCTTCATGTATAGAGT |
| | TTCTCCCTTGACCTACATGATTGATGCTTTGTTGGCTATTGGTGTCGGTAA |
| | CGTTGATATTGTCTGTACCAAGAATGAATTCGTGGAATTCACTCCAGCTG |
| | CTGGTATGACTTGTGGTGAATATTTGGCTCCATACTTGGCTTATGCTGGTA |
| | CTGGTTATTTGAGAGATAACAACGCTACTGATGTTTGCGGTTTGTGTGAAT |
| | ACTCTAGAACTAACGATTACTTGGCTACCGTTAATGCTGAATATGGTCAAC |
| | GTTGGAGAAACTACGGTATTTTCATCTGCTACATCTTCATTAACTTCGCCG |
| | CTGCTATCCTATTTTACTACTTAGCTAGAGTTCCCAAGAAGCAGAAAAAGT |
| | TGAAAGCCGAATGA |
| SEQ ID NO: | MSSSTNSDDESGRLHTYTGIDAQAEATIKNLARTLTAQSLNSINSSNNNNEDS |
| 1016 | TSVNDHQSIFSNLEGINPVLTNPEQSGYDEKLDPTSDYFSSTAWVKNMARLA |
| | KSDPDYYKPYSLGCVWKDLTASGTSSDVEYQATALNAPLKALSYLYREVKPV |
| | NEENSFQILKEMEGCINPGELLVVLGRPGSGCTTLLKTISTNTHGFHVGKNAT |
| | ISYSGFTPKEIKKHYRGEVVYNAESDIHLPHLTVYQTLYTVSRLKTPQNRIKGV |
| | DRDTFARHITEVAMATYGLSHTRDTKVGNDLVRGVSGGERKRVSIAEVSICG |
| | SKFQCWDNATRGLDSATALEFIRALKTDATTVNSCSTVAIYQCSQDAYDLFD |
| | KVCVLDQGYQIYFGPGTKAKAYFEKMGYVSPDRQTTADFLTAVTSPAERILN |
| | QEYLKKGISIPQTPEDMNTYWKNSQDYKDLMAEIDEKLNNNVEDSRELVKEA |
| | HIAKQSNRSRPSSPYTVNYFLQIKYLLTRNVWRIKNNPSVNLFMVFGNSSMA |
| | LILGSMFYKVMLHDSTSTFYFRGSAMFFAILFNAFSCLLEIFTLYEARPITEKHK |
| | TYSLYHPSADAFASIISELPTKAAIAVCFNIIFYFLVNFRRNGGDFFFYLLINIVA |
| | VFAMSHMFRTVGAATRTLSEAMFPASMLLLAMSMYTGFAIPKTKMLGWSEW |
| | IWYLNPLAYLFESLMINEFHNRDFPCAQYVPQGPAYVNATGTERVCISLGAVP |
| | GEDFVNGDAYIKANYGYEHKHKWRGFGVGMAFAVFFLGAYLAVCEFNEGAK |
| | QKGEILVFPKSIIRKMKKNGQLPQRARPDKREEDIEKGTTDESSVNDRKLLED |
| | SEISSDENKEESIGLSKSTAIFHWRNLCYDVQIKDETRRILSDVDGWVKPGTL |
| | TALMGASGAGKTTLLNCLAERVTMGTITGDVFVDGRLRDESFPRSIGYCQQQ |
| | DLHLRTSTVRESLRFSAYLRQPSDVSTEEKNAYVEEIITILEMDNYADAIVGVP |
| | GEGLNVEQRKRLTIGVELAAKPKLLVFLDEPTSGLDSQTAWAICQLMRKLAS |
| | HGQAILCTIHQPSAILMQEFDRLLFLQRGGKTVYFGELGHGCQKMIDYFESH |
| | GSHKCPPEANPAEWMLEVVGAAPGSHANQDYYEVWRNSEHFKAVHEELDR |
| | MEKDLPGQAKEEDDPTAHKEFATGIPYQINLVSERLFQQYWRSPQYLWSKFI |
| | LTIFNMLFIGFTFFKANTSLQGLQNEMLAIFMFTVIFNPLLQQYLPNFVEQRDL |
| | YEARERPSRTFSWKAFIVSQILVEVFWNFLAGSLAYFIYYYAIGFYHNASVAG |
| | QLHERGALFWLFSTAFFVYCGSMGTMAISFIEIAENAANMASLMFTMCLSFC |
| | GVMVTKDAMPRFWIFMYRVSPLTYMIDALLAIGVGNVDIVCTKNEFVEFTPAA |
| | GMTCGEYLAPYLAYAGTGYLRDNNATDVCGLCEYSRTNDYLATVNAEYGQR |
| | WRNYGIFICYIFINFAAAILFYYLARVPKKQKKLKAE- |
| SEQ ID NO: | ATGTGCTACCACTGCCTGATTTTGTTGTCCAGATTCATCGATTCCCTGATTCCAATCT |
| 1017 | TGTTCTTGTTGGGTTCTTTGACCTTTATCTTGCAAGCTTGGTTGAGATCCAGAAGATC |
| | TTTTCATTCTGCTGTTTACGCTCCATTGAAGCAACATGTTACATCTTATGGTACTACC |
| | CCATCTTTGGAAGAGGATGAAGATGATACTCCATCCTCATCTACTATTGCATCTGAA |
| | GAGTTGTACGATGTCCCATTGAATAGATGGTCTATCTACAACATTACCAGATTGGCT |
| | GGTACTTTGTTGCAATTGGGTATTTCTGCTGGTGTTTTGAGAGCTTTGTTGGTTCAT |
| | GATTACACTTTGGACACTACCGTTGAAGGTTCTTCTTCTAACGTTTTGACTTCCTCTA |
| | TGACCGATTGTGTTTATTGGGGTAGACCAATGTCTACTGAAATGTGGGCTTCTATCT |
| | ACTCCAAGTTTATGTTTTCTTGGGTTGCCCCAATGTTGAAAGAAGGTTATCAAAGAA |
| | CTTTGAACGCCGATGATTTGGTCGAATTGATTCCACAAAACAGAGCTAAGAACGTCT |
| | TGCAAACTTACCAACACTATAGGTCACCATCTTTGTTCTGGGGTGTTATCAGAACTTT |
| | CAAGAAAGAATTGGCCATTCAAGCTTTGTGGTGTGTTTTATGGAATGCTTGTTTGTT |
| | TGGTCCACCAGTGTTTCTGAACAAGATCATTAAGTACATCGAAAACAGACACCCAG |
| | ATCAACCAGTTTCTATGGCTTATGCTTACGTCATCTTGTTGTTCATTACCTCTGCTTCT |
| | CAAGCTTTAGCTTTACAACAAGCCTTGTACATTGGTAGAACTTTGGGTGTTAGAATC |
| | CAAGCCATTATAGTCGGTGAAGTTTTCTCTAAAGCTTTGCGTAAAAGACAGGGTGA |
| | TAGAGAAGAAACTGCTCACGATGATGATAACGACGAAAAGAAAGCTGAATCCAAC |
| | GTCAACAACTTGTTGTCCGTTGATTCCTTGAGAATCTCTGATTTTATGGCCTACTCTT |
| | TCCAGTTGTACGGTTCTGCTATTCAAATGGTTGTTTCAGGTGCCCTGTTGTACAATTT |
| | GTTGGGTACTGCTGCTTTGTGGGGTTTAGGTGTTATGGTTTTGACACAACCAGTTGC |
| | TTTCTTGGTGTCTAAGAGATTCGAAAAGGTTCAAGACCAAGTTATGTCTGCTACCGA |
| | TAACAGAATCAAAAAGGTCAACGAATTGCTGTCCGCCATTAGAATTATCAAGTTTTT |
| | CGCTTGGGAGAAAGAATTCAAAAAGCGTGTTATGGACGCCAGGGAAAAAGAATTG |
| | AAAGTTTTGTTGGCTAGGCTGTACATGCACGTTCATACTGTTAATGTTTGGTTCTTCA |
| | TCCCCATCCTGATTATGATTACTGTTTTCTACGCTTACACCAGGACTAATGATTTGAC |
| | TGCTGCTACTGCTTTTACAGCTTTGGCTTTGTTCAACATTTTGAGATTCGCCTTGGAT |
| | GAGTTGCCAATGTTTATTATGTGGGCATTGCAAGGTAGAGTTTCCGCTAAAAGAGT |
| | CCAAAAGTTTTTGGCCGAAGAAGAAGTTACTTCTCCACAACCTACTGTTATGCATGC |
| | TGATATTGGTTTCGTTGATAATGCTGCTTTCGGTTGGGAAAAAGATAAGGCCATTAT |
| | CAAGGACCTGAACTTGTCTTTTCCAAGGGGTAAGTTGTCTGTTATTTGTGGTCCAAC |
| | TGGTTCTGGTAAAACAACTTTGTTAGCCTCCTTGTTAGGTGAAACTTACTGTATGAG |
| | AGGTGCTGCTCATTTGCCAAGAATAGTTCCAAAATCTAAAGCTCCAGTTGGTGGTGC |
| | TGCTTCTGGTATTGCTTATGTTGCTCAAACTGCTTGGTTGCAGAACAGATCTATTAG |
| | AGACAATATCTTGTTCGGCTTGCCATACGATGCTGAAAGATATGAACAAGTGTTGTA |
| | CATTACTGCCTTGACCAAGGATTTGGAAATCTTCGAATATGGTGACTCCACCGAAAT |
| | TGGTGAAAAAGGTGTTACTTTGTCTGGTGGTCAAAAGCAAAGAATGGCTATTGCTA |
| | GAGCTGTTTACTCTCAAGCCGAAACCATTATTTTGGATGATTGCTTGTCTGCTGTTG |
| | ATGCTCATACTGCTAAACACTTGTACGAACATTGCATCACTGGTAACTTGATGAAGG |
| | ATAGAACCGTTATCTTGGTTACCCATCATGTTGCATTGTGTTTGGATTCTGCTGCATA |
| | TGTTGTTGCTATGAAGGATGGTCAAGTTTTAGGTGCTGGTGATCCATCTACTGTTTT |
| | GAAATCTGGTGCTTTGGGTGAAGAATTAGCTAACACTTACGACGAGGATAGGTCCT |
| | CTAAGAAAGAAAGTGCTGGTGCTACTGATGGTGCTGTTCCATTGGTTCCAGAAACT |
| | GTTGAATTGGATAAGAAAGACGGTACTGGCAAATTGGTAAAAGAAGAAGAGAGAG |
| | CTGAAGGTTGGGTTCCATGGTCAATCTATGAAGCTTATATCTACGCTTCTGGTGGCT |
| | ATATTTTCTGGGCCTTGATTTTGATTGCCTTCTGCTTGAATCAAGGCGTTATTTTCTC |
| | ACAAGACTACTGGATTAAGGTTTGGACATCTGCTTACGAAACTAAGAACCATGATTT |
| | CGGTTTGTTGAACGCTTTGGGTACTAGATCCTCCATCTTATTGAACAGCTTGTCCTTG |
| | TCATCTGTCTCTCCAAGTGAAGAACCTAATCAACAACAAAGGGTTAACGTCATGTAC |
| | TACTTGGGTATCTACACCTTGATTGGTGTCATGGCTTTATTGGCTACTACTGCTAGAT |
| | CTTTGATCGTTTACTACGGTGGTATTAGAGCCTCTAGAAGATTGCACGAAAAGTTGT |
| | TGCATAAGATCTTGAGAGCCAAGGTTAGATTCTTTGATTCTACTCCTTTGGGCAGAA |
| | TCTTGAACAGATTCTCTACTGATATGGAAGCCATCGATCAAGAGGTTTCTCCAATGG |
| | TTGCTTTCGTTTTGTTCTCTATGGTTACCATGTTCTGCGTCTTGATCTTGGTTTCTACA |
| | GTTTTGCCAGCATTCTTGATTCCAGGTACTTTCATTGCTGTCGTGTTCGTTTTTATCG |
| | GCATGTATTATTTGGCCACCTCCAGAGATTTGAAGAGATTGAATTCTGTGTCCAGAT |
| | CTCCAATCTACGTCCAATTTTCTGAAACCTTGAACGGTGTTTCTACCATTAGAGCTTT |
| | TGGTGCTCAACCACGTTTCATCAAAGAAAACCATGAATTGGTCGACAACAACAACA |
| | GACCATTTTTGTGGATGTGGGCTACAAACAGATGGTTGCATAGTAGAATAGAAGTT |
| | TTGGGTTCCGCTGTCTGTTTCTTTACTGGTTTGGTTATTATGATCGCCAGATCCTGGA |
| | TTGATCCAGGTTTGGCTGGTTTGTGTTTGTCTTATGCTTTACAATTCACCGACAACTT |
| | GATTTGGGTTGTCAGAGGTTATGCTCAGAACGAAATGAACATGAACTCTATCGAAC |
| | GTGTGCAAGAGTACATGGACATTGAAGAAGAGGCTCCAGAACATATTCCAGCTACT |
| | TGTCCACCAAAATCTTGGCCAGAAACAGGTGCTGTTAAGGTTGAAGAATTGGAAAT |
| | GAGATACTCCCAAGATTCTCCAGCTGTCTTGCATAGAATTTCTTTCGAAACAAAGCC |
| | CAGAGAAAAGGTTGGTATTGTCGGTAGAACAGGTTCAGGTAAATCTACTTTGGCCT |
| | TGTCTTTGTTCAGGTTCATGGAAGCTAGTCATGGTAGAATTCTGATTGATGGTGTTG |
| | ATATCGCCAACCTTGGTTTGGAAGATTTGAGATCTAGACTGACCATCATTCCACAAG |
| | ATCCAGTTTTGTTTTCCGGTACTTTGAGGTCTAATTTGGATCCATTTGGTCAACATGA |
| | CGATGCAGAATTGTGGGCTGCTTTAAAAAGATCCCACTTGATTGATCAACAGGATG |
| | GTGACGAAGAGAAGAAGATTACCTTAGATTCCGTTGTCTTGGAGAATGGTAACAAT |
| | TGGTCACAAGGTCAGAGACAATTGATTGCTTTGGCTAGAGCTTTGGTTAAGAAGTC |
| | CACTTTGATCATCTTGGATGAAGCTACCTCTTCTGTTGATTTCGATACCGATAGAAA |
| | GATCCAAGAAACCATCAGGTCTGAATTGGTTCACTCTTCTTTGTTGTGCATTGCCCAT |
| | AGAATTAGAACTGTTGCCGATTACGATAGGATTTTGGTTTTGGATCAGGGTAGAGT |
| | CAAAGAGTTTGATACCCCATACAATTTGATCACCAGGCAAGATTCCATCTTCAGACA |
| | AATGTGTCAAAGGTCTGGTGAATTCGAAGAGTTATTGGAAATTGCTTCTGCCAAAC |
| | ATTCTGTCGCTTGA |
| SEQ ID NO: | MCYHCLILLSRFIDSLIPILFLLGSLTFILQAWLRSRRSFHSAVYAPLKQHVTSY |
| 1018 | GTTPSLEEDEDDTPSSSTIASEELYDVPLNRWSIYNITRLAGTLLQLGISAGVL |
| | RALLVHDYTLDTTVEGSSSNVLTSSMTDCVYWGRPMSTEMWASIYSKFMFS |
| | WVAPMLKEGYQRTLNADDLVELIPQNRAKNVLQTYQHYRSPSLFWGVIRTFK |
| | KELAIQALWCVLWNACLFGPPVFLNKIIKYIENRHPDQPVSMAYAYVILLFITSA |
| | SQALALQQALYIGRTLGVRIQAIIVGEVFSKALRKRQGDREETAHDDDNDEKK |
| | AESNVNNLLSVDSLRISDFMAYSFQLYGSAIQMVVSGALLYNLLGTAALWGL |
| | GVMVLTQPVAFLVSKRFEKVQDQVMSATDNRIKKVNELLSAIRIIKFFAWEKE |
| | FKKRVMDAREKELKVLLARLYMHVHTVNVWFFIPILIMITVFYAYTRTNDLTAA |
| | TAFTALALFNILRFALDELPMFIMWALQGRVSAKRVQKFLAEEEVTSPQPTVM |
| | HADIGFVDNAAFGWEKDKAIIKDLNLSFPRGKLSVICGPTGSGKTTLLASLLGE |
| | TYCMRGAAHLPRIVPKSKAPVGGAASGIAYVAQTAWLQNRSIRDNILFGLPY |
| | DAERYEQVLYITALTKDLEIFEYGDSTEIGEKGVTLSGGQKQRMAIARAVYSQ |
| | AETIILDDCLSAVDAHTAKHLYEHCITGNLMKDRTVILVTHHVALCLDSAAYVV |
| | AMKDGQVLGAGDPSTVLKSGALGEELANTYDEDRSSKKESAGATDGAVPLV |
| | PETVELDKKDGTGKLVKEEERAEGWVPWSIYEAYIYASGGYIFWALILIAFCL |
| | NQGVIFSQDYWIKVWTSAYETKNHDFGLLNALGTRSSILLNSLSLSSVSPSEE |
| | PNQQQRVNVMYYLGIYTLIGVMALLATTARSLIVYYGGIRASRRLHEKLLHKIL |
| | RAKVRFFDSTPLGRILNRFSTDMEAIDQEVSPMVAFVLFSMVTMFCVLILVST |
| | VLPAFLIPGTFIAVVFVFIGMYYLATSRDLKRLNSVSRSPIYVQFSETLNGVSTI |
| | RAFGAQPRFIKENHELVDNNNRPFLWMWATNRWLHSRIEVLGSAVCFFTGL |
| | VIMIARSWIDPGLAGLCLSYALQFTDNLIWVVRGYAQNEMNMNSIERVQEYM |
| | DIEEEAPEHIPATCPPKSWPETGAVKVEELEMRYSQDSPAVLHRISFETKPRE |
| | KVGIVGRTGSGKSTLALSLFRFMEASHGRILIDGVDIANLGLEDLRSRLTIIPQD |
| | PVLFSGTLRSNLDPFGQHDDAELWAALKRSHLIDQQDGDEEKKITLDSVVLE |
| | NGNNWSQGQRQLIALARALVKKSTLIILDEATSSVDFDTDRKIQETIRSELVHS |
| | SLLCIAHRIRTVADYDRILVLDQGRVKEFDTPYNLITRQDSIFRQMCQRSGEFE |
| | ELLEIASAKHSVA- |
| SEQ ID NO: | ATGTCCAACCAGGGTGATCATCACTTGAACAATGATTTGGTTGATGACGA |
| 1019 | CATCGAAAGATACCATGGTTTCGATTCTCAAGTCAACGAACAAATTACCAA |
| | CTTGGCTAGACAATTCACCAACGTTTCTGCTCAAGAGGCTGAAAAAGAAG |
| | ATGAAGGCGTTGAAGCTTTGTCTAGAGTTTCTACTGTTGCTGCTGGTGTT |
| | GATTTCATCAACAACGTTGATAACGATCCAAGATTGGACCCAAACAACGA |
| | TCAATTCAATTCCAGATTCTGGATCAAAAACATGAAGGCCTTGGTTGATAA |
| | TGACCCAGATTACTACAAGACCTACTCTTTGGGTATTACCTACAAGGATTT |
| | GAGAGCTTATGGTGTTGCTACTGATGCTGATTACCAAACTACTGTTGTTAA |
| | CGGCTTGATTAAGAACGCTAACAACGCTTTCAGATACCTGACCGAATCTA |
| | AAGAGACTAAGAAGAACAAGTACTTCAACATCTTGAAGCCAATGGATGCC |
| | TTGATTCAACCAGGTGAATTGGTTGTTGTTTTAGGTAGACCAGGTTCTGGT |
| | TGTTCTACTCTGTTGAAAACTATTGCCTCTAACACTCATGGTTTCCACATT |
| | GGTGAAGAATCCGTTATTTCTTACGAAGGCTTGACCCCAAAAGAAATCAA |
| | AGCTCATTATAGAGGTGAGGTTGTTTACAACGCCGAATCCGATATTCATTT |
| | CCCACATTTGACTGTTGGTCAGACTTTGAAAAACGCTGCTAAATTCAGAAC |
| | CCCAAGAAACAGAATTCCAGGTGTTTCTAGAGATCAATACGCTGAATGTA |
| | TGACCGATGTTTACATGGCTACTTATGGTTTGTCTCATACCAAGAACACTA |
| | AGGTCGGTTCAGAATTGGTTAGAGGTGTTTCAGGTGGTGAAAGAAAGAGA |
| | GTTTCAATTGCCGAAGTTTCTTTGGCTGGTGCTAGATTGCAATGTTGGGA |
| | TAATGCTACTAGAGGTTTGGATGCTGCTACTGCTTTGGAATTCATTAGAGC |
| | TTTGAGAACCTCCGCTGATATTTTGGATACAACTGCTTTAGTTGCCATCTA |
| | CCAATGTTCTCAAGATGCCTATGATCTGTTCGATAAGGTTTCTGTCTTGTA |
| | CGAAGGTTACCAAATCTTCTTCGGTAGAGTTGAAGATGCCAAGCAATACT |
| | TTTTGAACATGGGTTATGATTGCCCAGCTAGACAAACTACAGCTGATTTCT |
| | TGACTTCTATTACCTCCGCCTCTGAAAGAATTGCTAGATCTGGTTTTGAAA |
| | ACAAGGTCCCAAAAACTCCAGCTGAATTCGAACAATATTGGAGACAATCT |
| | CCAGAGTACACCCAATTGATTTCCGATATCGATTCCAACTTGATCCAGGC |
| | CAAAGAATCTAACATTGCCCAAGATATTAAGATCGCTCATAACGCTAAGCA |
| | GTCCAAGAGAATGAGACATTCTTCTCCATACACTGTTTCTTTTGCTATGCA |
| | GACCAAGTACCTGTTGATCAGAGAATACCAGAGAATCTACAACGATGTTT |
| | TCGTTACTGCCTTCTCCGTCTTTTTCAACTCTTTGATGTCTCTGGTCCTGT |
| | CCTCTATTTTCTATAACTTGCAACAAAACACCGACAGCTTTTACAAAAGGG |
| | GTGCTGCTATGTTTTTCAGCGTTTTGTTTAACGGTTTCCTGTCCTTCTTGG |
| | AAATCATGACTTTGTTTGAAGCCAGACCAGTTGTTGAAAAGCACAAGCAAT |
| | ATTCGTTGTACCATCCATCAGCTAATGCTTTGGCTTCTGTCATTTCTCAGA |
| | TTCCTTTCAAGTTGGTTACCTCCTTGGCTTTCAACCTGATTTTCTACTTCAT |
| | GGTCCACTTCAGAAGAGAACCAGGTAGATTTTTCTTCTACATGCTGATGA |
| | ACTTGACTGCTACTTTCGCTATGTCCCATTTGTTTAGATTGTGTGGTTCTG |
| | CTGCTTCTTCATTGGCTGAAGCTATAGTTCCAGCTCACTTTTTGTTGTTGG |
| | CTTTGACTATTTTCGTCGGCTTTACCATTCCAGTCAACTATATGTTAGGTT |
| | GGTCCAGATGGATCAACTACTTGGATCCATTAGCTTACGCTTTCGAAGCT |
| | TTGATGGCTAACGAATTTTCTGGTTTGGAATACGCTTGCTCCTCTTATATT |
| | CCAGCTGCTCCAAATGGTATTACTGAAGGTGATACTCATTACGTCTGTAAT |
| | GCTGTTGGTGCTGTTCCAGGTAAATCTTTTGTTTCTGGTACTAGATACTTG |
| | GAGGTTGCCTACAAGTATACCAATTCTCATAAGTGGCGTGACTTCGGTAT |
| | TTTGTTAGGTTTGACTTTATTCCCATTGGGCGTCTACATGTTCTTGTCTGA |
| | GTATAATGAATCCGCCAAGCAAAAGGGTGAAGTCTTGTTGTTTCAAAGGT |
| | CTACCTTGAAGGACATCAAAAAGAACAACAAGGTCATCAACGATATCGAA |
| | GCTGGTGACGAAAAAGATGTTTCCTTGCAAGATGAATCCACAGAAGGTGA |
| | CATTAAGGCTTTAGATGCTGGTAAGGATATCTTCCATTGGAGAGATGTTA |
| | GATACACCGTCCAGATCAAGAAAGAAGAAAGACAAATCTTGTCCGGTGTT |
| | GATGGTTGGGTTAAGCCAGGTACTTTGACTGCTTTAATGGGTGCTTCTGG |
| | TGCTGGTAAAACTACCTTGTTGGATGTTTTGGCTAACAGAGTTACTATGG |
| | GTGTTGTTACTGGTGATATGTTCGTTAACGGTAGATTGAGAGACTCCTCAT |
| | TCCAAAGATCTACTGGTTACGTTCAACAACAGGACTTGCACTTGCAAACT |
| | GCTACTGTTAGAGAAGCCTTGAAGTTTTCTGCTTACTTGAGACAACCTAGA |
| | GATGTCACTAGAGAAGAAAAGGATAACTACGTCGAAGAAGTCATCAAGAT |
| | TTTGGAGATGGAAAAGTACGCTGATGCTGTTGTTGGTGTTGCCGGTGAAG |
| | GTTTGAATGTTGAACAAAGAAAAAGGTTGACCATCGGTGTTGAATTGGCT |
| | GCTAAACCTAAGTTGTTGCTGTTTTTGGATGAACCTACCTCCGGTTTGGAT |
| | TCACAAACTGCTTGGTCTATTTGCCAGTTGATGAGAAAGTTGGCTAATCAT |
| | GGTCAAGCTATTTTGTGCACTATCCATCAACCATCCGCAATTTTGATGCAA |
| | GAATTCGATAGGTTGCTATTCTTAGCTAGAGGTGGTAAGACTGTTTACTTT |
| | GGTGATTTGGGTAAGAACTGCCAAACCTTGATTGATTACTTTGAGAAATAC |
| | GGTGCTCCAAAGTGTCCAGGTGATGCTAATCCAGCAGAATGGATGTTGCA |
| | TGTTATTGGTGCAGCTCCAGGTTCTCATGCTAATCAAGATTATCATGAAGT |
| | CTGGCTGAACTCCACTGAAAGACAAGCTGTTTTAGATGAGTTGGACTACA |
| | TGGAAAGGGAATTAGTTAAGATCCCAGTTGATAACTCCGTGGACAATTAT |
| | GAATTTGCTGCTCCATTCACTACCCAATACGCTATCGTTACCAAAAGAGTG |
| | TTTCAGCAGTATTGGAGAACTCCAATCTACATCTGGTCCAAGTTGTTTTTG |
| | GCTACTGTTCCATCTTTGTTCTTGGGTTTTGCTTTCTTTAAGGCCACCAAT |
| | TCATTGCAGGGTTTCCAAAATCAAATGTTCGCCCTGTTTATGTTCCTGGTC |
| | ACTTTTAATCCTTTGGTCCAACAAATTATCCCAGCCTTTGTTGTTCAGAGG |
| | GACTTATACGAAACTAGAGAAAGACCATCTAAGACCTTTTCTTGGATTGCC |
| | TTCATTTTGTCTCAATTCACTGCTGAATTGCCATGGAATGCATTTGTTGGT |
| | ACTATCGCTTTCTTCGTGTTTTATTACCCAGTCGGTTTTTACAACAACGCC |
| | AAGTATGATCATTTGACCAATGAGAGAGGTGCTTACGCCTGGTTTTTCACT |
| | GTTTTATTCTACGTCTACTCCGGTTCTATGGCTCATTTGTTGATTAGCCCA |
| | ATTCAAATTGCTGATGCAGCAGGTAATTTGGGCTCTTTGTTATTCACTATG |
| | TCCTTGAATTTCTGCGGTGTTTTAGTAGGTCCTACTCAATTTCCAGGTTTC |
| | TGGATTTTCATGTACAGAGTTTCACCATTCACCTACTTCATCGATGGTTTC |
| | TTGTCAAATGCTTTAGCTCATACCACCGTTCAATGTTCAGCTGCAGAATTA |
| | GTAACTATGGAACCTACTGCTGGTTTGACATGTGGTGATTATCTGTCATCT |
| | TACATTGCTGCTGCAGGTACTGGTTATGTAAACAATCCAGATGCTACTTCT |
| | GCTTGCCAATTTTGCTCTGTTTCAACTTCTGATGCCTACCTGAAACTGATC |
| | TCTTTGTCTTACTCTAGAAAGGGTAGAAACATGGGTGTTTTCGTCGCTTAC |
| | ATTTTCATTAACTGGGCCTTAGCTATCTTCTTCTATTGGTTGGCTAGAGTC |
| | CCTAAGAAAAACAATAGAGTCAAGGATGAGAGGGACCCTAACAAGAAAAA |
| | GTCAGAGATCAAAGAATGA |
| SEQ ID NO: | MSNQGDHHLNNDLVDDDIERYHGFDSQVNEQITNLARQFTNVSAQEAEKEDEGVEAL |
| 1020 | SRVSTVAAGVDFINNVDNDPRLDPNNDQFNSRFWIKNMKALVDNDPDYYKTYSLGITY |
| | KDLRAYGVATDADYQTTVVNGLIKNANNAFRYLTESKETKKNKYFNILKPMDALIQPGE |
| | LVVVLGRPGSGCSTLLKTIASNTHGFHIGEESVISYEGLTPKEIKAHYRGEVVYNAESDIHF |
| | PHLTVGQTLKNAAKFRTPRNRIPGVSRDQYAECMTDVYMATYGLSHTKNTKVGSELVR |
| | GVSGGERKRVSIAEVSLAGARLQCWDNATRGLDAATALEFIRALRTSADILDTTALVAIY |
| | QCSQDAYDLFDKVSVLYEGYQIFFGRVEDAKQYFLNMGYDCPARQTTADFLTSITSASE |
| | RIARSGFENKVPKTPAEFEQYWRQSPEYTQLISDIDSNLIQAKESNIAQDIKIAHNAKQSK |
| | RMRHSSPYTVSFAMQTKYLLIREYQRIYNDVFVTAFSVFFNSLMSLVLSSIFYNLQQNTD |
| | SFYKRGAAMFFSVLFNGFLSFLEIMTLFEARPVVEKHKQYSLYHPSANALASVISQIPFKL |
| | VTSLAFNLIFYFMVHFRREPGRFFFYMLMNLTATFAMSHLFRLCGSAASSLAEAIVPAHF |
| | LLLALTIFVGFTIPVNYMLGWSRWINYLDPLAYAFEALMANEFSGLEYACSSYIPAAPNGI |
| | TEGDTHYVCNAVGAVPGKSFVSGTRYLEVAYKYTNSHKWRDFGILLGLTLFPLGVYMFL |
| | SEYNESAKQKGEVLLFQRSTLKDIKKNNKVINDIEAGDEKDVSLQDESTEGDIKALDAGK |
| | DIFHWRDVRYTVQIKKEERQILSGVDGWVKPGTLTALMGASGAGKTTLLDVLANRVT |
| | MGVVTGDMFVNGRLRDSSFQRSTGYVQQQDLHLQTATVREALKFSAYLRQPRDVTRE |
| | EKDNYVEEVIKILEMEKYADAVVGVAGEGLNVEQRKRLTIGVELAAKPKLLLFLDEPTSGL |
| | DSQTAWSICQLMRKLANHGQAILCTIHQPSAILMQEFDRLLFLARGGKTVYFGDLGKN |
| | CQTLIDYFEKYGAPKCPGDANPAEWMLHVIGAAPGSHANQDYHEVWLNSTERQAVL |
| | DELDYMERELVKIPVDNSVDNYEFAAPFTTQYAIVTKRVFQQYWRTPIYIWSKLFLATVP |
| | SLFLGFAFFKATNSLQGFQNQMFALFMFLVTFNPLVQQIIPAFVVQRDLYETRERPSKTF |
| | SWIAFILSQFTAELPWNAFVGTIAFFVFYYPVGFYNNAKYDHLTNERGAYAWFFTVLFY |
| | VYSGSMAHLLISPIQIADAAGNLGSLLFTMSLNFCGVLVGPTQFPGFWIFMYRVSPFTYF |
| | IDGFLSNALAHTTVQCSAAELVTMEPTAGLTCGDYLSSYIAAAGTGYVNNPDATSACQF |
| | CSVSTSDAYLKLISLSYSRKGRNMGVFVAYIFINWALAIFFYWLARVPKKNNRVKDERDP |
| | NKKKSEIKE- |
| SEQ ID NO: | ATGTCCGAATTGGACAAGGACTCCTCTTCTAATGTTTCTTCTGCCTCCGAT |
| 1021 | CATATCTCTTACAATGGTTTTGACGAAAAGGCCGACAAGAGAATTAGACA |
| | ATTGGCTCAACAATTGACCAGAACCTCTACTAATTTGGATACCCAAGATGT |
| | CAACGGTAACAACAACAACGATGTTCAATCTACCGTGTCCAGATCCATTTT |
| | CTCTGACAGTTTGTTGAAAGAAGGTGTCAACCCAGTTTTCACCGATATTAA |
| | CGATCCAAACTACAACGAAAAGCTGGACCCAAATTCTGACAACTTCTCTT |
| | CTTATGATTGGGTCAAGAACATCTCCAATTTGGCTAATGCTGATCCAGATT |
| | ACTACAAGCCATACAAGTTGGGTTGTTCTTGGAAGAATTTGACTGCTGAA |
| | GGTAACTCCTCTGATGTCTCTTATCAATCCACTGTTTTGAACCTGCCATTG |
| | AAGTTGGCTACTTTGGGTTATTACTTGTTGTCATCTGGTGCCAACAAGAAG |
| | GTTCAAATCTTGAAATCTGTTGACGGTTTGATTAAGCCAGGTGAGTTGTTG |
| | GTTGTTTTGGGTAGACCAGGTTCAGGTTGTACTACTTTGTTGAAGTCTATT |
| | ACCTCTAACACCCACGGTTTCCAATTGACTGATGAATCCGAAATTTCCTAC |
| | GATGGTTTGACCCCAAAAGAGATCAAAAAGCACTATAGAGGTGATGTTGT |
| | TTACAACGCTGAAGCCGATATTCATTTGCCACATTTGACTGTTTTCCAAAC |
| | CTTGGTTACTGTCGCTAAGTTGAAAACTCCACAGAATAGATTCAAGGGTG |
| | TCACCAGAGAACAATTCGCTGATCATGTTACTGATGTTACTATGGCTACTT |
| | ACGGTTTGTTGCATACCAGAAATACCAAGGTTGGTAACGATTTGGTTAGA |
| | GGTGTTTCTGGTGGTGAAAGAAAGAGAGTTTCTATTGCCGAAGTTACCAT |
| | TTGCGGTTCTAAGTTTCAATGTTGGGATAACGCTACTAGAGGTTTGGATTC |
| | TGCTACTGCTTTGGAATTCATTAGAGCCTTGAAAACACAAGCCGTCTTGC |
| | AAAACACTGCTGCTACTGTTGCTATCTACCAATGTTCTCAAGATGCCTACG |
| | ATTTGTTCGATAAGGTTTGCGTTTTGGATGAAGGTTACCAGTTGTTTTACG |
| | GCTCATCTTCTAAGGCCAAAGAATTTTTCATCAAGATGGGTTACATTTGCC |
| | CACCAAGACAAACTACTGCTGATTTCTTGACTTCTGTTACCTCTCCAGTTG |
| | AAAGGATCTTGAACGAAGAATACTTGGCTAAGGGTATCAAGATTCCACAA |
| | ACTCCTAGAGACATGAGCGAATATTGGAGAAATTCTCAAGAGTACAGAGA |
| | CTTGATCAGAGAAATCGATGAGTACAACGCTCAAAACAACGACGAATCCA |
| | AGCAAATTATGCATGATGCTCATGTTGCTACCCAATCTAGAAGGGCTAGA |
| | CCATCTTCTCCATATACTGTTTCTTATGGCTTGCAGATCAAGTACATCTTG |
| | ACTAGAAACATCTGGCGTATGAAGAACTCCTTCGAAATTACTGGTTTCCA |
| | GGTTTTTGGTAACTCTGCTATGGCTTTGATTTTGGGCTCTATGTTCTACAA |
| | GGTTATGTTGCATCCAACTACCGATACCTTTTATTACAGAGGTGCTGCTAT |
| | GTTCTTCGCCGTTTTGTTTAATGCCTTCTCATCCTTGATTGAGATCTTCAC |
| | CTTGTATGAAGCTAGACCAATTACCGAAAAGCACAAGTCCTACTCATTATA |
| | CCATCCATCTGCTGATGCTTTCGCCTCCATTATTTCTGAAATTCCTCCAAA |
| | GTTGATCACCTCCGTTTGCTTCAACATTATCTTCTACTTTCTGTGCAACTT |
| | CAGACGTAATGGTGGTGTTTTCTTCTTCTACTACCTGATTTCTATCGTTGC |
| | TGTTTTTGCCATGTCTCACTTGTTTAGATGTGTTGGTTCTTTGACCAAGAC |
| | CTTGCAAGAAGCTATGGTTCCAGCTTCTATGTTGTTATTGGCTTTGTCTAT |
| | GTACACCGGTTTCGCTATTCCTAGAACTAAGATTTTAGGTTGGTCCATCTG |
| | GGTTTGGTACATTAACCCATTGGCTTACTTGTTCGAGTCCTTGATGATTAA |
| | CGAATTCCATGGTAGACATTTCCCATGCACTGCTTATATTCCAGCTGGTG |
| | GTTCTTACGATTCTCAAACTGGTACTACCAGAATCTGTTCTGTTAATGGTG |
| | CTATTGCTGGTCAAGATTACGTTTTGGGTGACGATTACATCAAATCCTCTT |
| | ATGCTTACGAACACAAACATAAGTGGCGTGGTTTTGGTGTTGGTATGGCT |
| | TATGTTGTTTTCTTTTTCGTCGTCTACTTGGTCATTTGCGAGTATAATGAAG |
| | GTGCTAAGCAAAAGGGTGAGATCTTGGTTTTTCCAAGATCCGTTGTTAAG |
| | AAGATGAAGAAAGCTAAGACCCTGAACGACTCTTCTTCAAACGTTTCTGAT |
| | GTTGAAAAGGCCACCTCCGAATCTATTTCTGATAAGAAGTTGCTGGAAGA |
| | GTCCTCTGGTTCTTTTGATGATTCTTCCGAAAGAGAGCACTTCAACATCTC |
| | TAAATCTTCCGCTGTTTTTCACTGGCGTAACTTGTGTTATGATGTCCAAAT |
| | CAAGTCCGAAACCAGACGTATTTTGAACAATGTTGATGGTTGGGTTAAGC |
| | CTGGTACTTTAACTGCTTTGATGGGTTCTTCTGGTGCTGGTAAAACTACTT |
| | TATTGGATTGCTTGGCTGAAAGGGTTACCATGGGTGTTATTACTGGTGAT |
| | ATTTTTGTGGACGGTTTGCCAAGAGATACTTCATTCCCAAGATCTATCGGT |
| | TACTGCCAACAACAAGACTTGCATTTGACTACTGCTACCGTTAGAGAATC |
| | CTTGAGATTTTCTGCTGAATTGAGACAACCAGCCGATGTTTCTGTTTCTGA |
| | AAAACATGCTTACGTCGAAGAGGTCATTAAGATTTTGGAGATGGAAAAGT |
| | ACGCTGATGCTGTTGTTGGTGTTGCTGGTGAAGGTTTGAACGTTGAACAA |
| | AGAAAAAGATTGACCATCGGTGTAGAATTGGCTGCTAAACCTAAGTTGTT |
| | AGTTTTCTTGGATGAACCCACTTCAGGTTTGGACTCTCAAACAGCTTGGT |
| | CTATTTGTCAGCTAATGAAGAAGTTGGCCAAGTTCGGTCAAGCTATTTTGT |
| | GTACTATTCATCAACCATCCGCCATCCTGATGCAAGAATTCGATAGACTGT |
| | TGTTCCTACAGAAAGGTGGTAAGACTGTTTACTTTGGTGAATTGGGTGAT |
| | AACTGCACCACCATGATTGATTACTTTGAAAGAAACGGTGCTCATAAGTGT |
| | CCACCAGATGCTAATCCAGCTGAATGGATGTTGGAAGTTGTCGGTGCTGC |
| | TCCAGGTTCTCATGCTTCACAAGATTACAATGAAGTCTGGCGTAATTCCG |
| | ATGAGTATAGAGCTGTTCAAGAGGAATTGGATTGGATGGAATCTGAATTG |
| | CCAAAACAAGCTACTGAAACTTCTGCCCACGAATTATTGGAATTCGCTTCT |
| | TCTTTGTGGATCCAATATGTTGCTGTCTGCATTAGGTTGTTCCAACAATAT |
| | TGGCGTACCCCATCTTACATTTGGTCTAAGTTTTTGGTCACCATCTTCAAC |
| | GCTTTGTTCATCGGTTTTACTTTCTTCAAGGCTGACAGAACATTGCAGGGT |
| | TTACAAAATCAGATGTTGGCCATTTTCATGTTCACCGTTATTACGAACCCA |
| | ATCTTGCAACAATACTTGCCATCTTTCGTTACTCAGAGAGACTTATACGAA |
| | GCTAGAGAAAGACCATCCAGAACATTCTCTTGGAAGGCTTTTATTGCTGC |
| | CCAAATCTCTGTTGAAATCCCATGGTCAATTTTGGCCGGTACTCTATATTT |
| | CCTGATCTACTATTACGCCATCGGCTTTTACAACAATGCTTCTGCTGCTGA |
| | TCAATTGCACGAAAGAGGTGCTTTGTTTTGGTTATTTTCTTGCGCCTTCTT |
| | CGTCTACATCGTTTCTTTGGGTACTTTGGTCATTGCCTTCAATCAAGTTGC |
| | TGAAACTGCTGCTCATTTGGCTTCATTGATGTTTACCATGTGCTTGTCTTT |
| | CAACGGTGTCTTAGTTACTTCTGCTAAGATGCCAAGATTCTGGATCTTCAT |
| | GTATAGGGTTTCTCCATTCACCTACTTCGTTGATGCTTTATTGTCTACCGG |
| | TGTTGCTAACGTTGAAGTTCATTGTGCTGATTACGAATTGAGAAAGTTCAC |
| | TCCACCATCTGGTTTGACTTGTGGTGAGTACATGGATCCTTATATTTCTGC |
| | TGCAGGTACTGGTTATTTGACTGATCCAAACAATACCGAAACCTGCTCTTT |
| | CTGTCAAATTTCTGACACCAATACTTTCTTGGCCTCTGTTGGTTCATCTTA |
| | TCATAGACGTTGGAGAAACTTCGGTATCTTCATTTGTTTCATTGCCATCAA |
| | CTACATCGGCGGCATTTTCTTTTATTGGTTGTCTAGAGTTCCAAAGGGCTC |
| | CAAAAAGATGAAGTCTAAGTGA |
| SEQ ID NO: | MSELDKDSSSNVSSASDHISYNGFDEKADKRIRQLAQQLTRTSTNLDTQDVNGNNNN |
| 1022 | DVQSTVSRSIFSDSLLKEGVNPVFTDINDPNYNEKLDPNSDNFSSYDWVKNISNLANAD |
| | PDYYKPYKLGCSWKNLTAEGNSSDVSYQSTVLNLPLKLATLGYYLLSSGANKKVQILKSV |
| | DGLIKPGELLVVLGRPGSGCTTLLKSITSNTHGFQLTDESEISYDGLTPKEIKKHYRGDVVY |
| | NAEADIHLPHLTVFQTLVTVAKLKTPQNRFKGVTREQFADHVTDVTMATYGLLHTRNT |
| | KVGNDLVRGVSGGERKRVSIAEVTICGSKFQCWDNATRGLDSATALEFIRALKTQAVLQ |
| | NTAATVAIYQCSQDAYDLFDKVCVLDEGYQLFYGSSSKAKEFFIKMGYICPPRQTTADFL |
| | TSVTSPVERILNEEYLAKGIKIPQTPRDMSEYWRNSQEYRDLIREIDEYNAQNNDESKQI |
| | MHDAHVATQSRRARPSSPYTVSYGLQIKYILTRNIWRMKNSFEITGFQVFGNSAMALIL |
| | GSMFYKVMLHPTTDTFYYRGAAMFFAVLFNAFSSLIEIFTLYEARPITEKHKSYSLYHPSA |
| | DAFASIISEIPPKLITSVCFNIIFYFLCNFRRNGGVFFFYYLISIVAVFAMSHLFRCVGSLTKTL |
| | QEAMVPASMLLLALSMYTGFAIPRTKILGWSIWVWYINPLAYLFESLMINEFHGRHFPC |
| | TAYIPAGGSYDSQTGTTRICSVNGAIAGQDYVLGDDYIKSSYAYEHKHKWRGFGVGMA |
| | YVVFFFVVYLVICEYNEGAKQKGEILVFPRSVVKKMKKAKTLNDSSSNVSDVEKATSESIS |
| | DKKLLEESSGSFDDSSEREHFNISKSSAVFHWRNLCYDVQIKSETRRILNNVDGWVKPG |
| | TLTALMGSSGAGKTTLLDCLAERVTMGVITGDIFVDGLPRDTSFPRSIGYCQQQDLHLT |
| | TATVRESLRFSAELRQPADVSVSEKHAYVEEVIKILEMEKYADAVVGVAGEGLNVEQRK |
| | RLTIGVELAAKPKLLVFLDEPTSGLDSQTAWSICQLMKKLAKFGQAILCTIHQPSAILMQE |
| | FDRLLFLQKGGKTVYFGELGDNCTTMIDYFERNGAHKCPPDANPAEWMLEVVGAAPG |
| | SHASQDYNEVWRNSDEYRAVQEELDWMESELPKQATETSAHELLEFASSLWIQYVAV |
| | CIRLFQQYWRTPSYIWSKFLVTIFNALFIGFTFFKADRTLQGLQNQMLAIFMFTVITNPIL |
| | QQYLPSFVTQRDLYEARERPSRTFSWKAFIAAQISVEIPWSILAGTLYFLIYYYAIGFYNNA |
| | SAADQLHERGALFWLFSCAFFVYIVSLGTLVIAFNQVAETAAHLASLMFTMCLSFNGVL |
| | VTSAKMPRFWIFMYRVSPFTYFVDALLSTGVANVEVHCADYELRKFTPPSGLTCGEYM |
| | DPYISAAGTGYLTDPNNTETCSFCQISDTNTFLASVGSSYHRRWRNFGIFICFIAINYIGGI |
| | FFYWLSRVPKGSKKMKSK- |
| SEQ ID NO: | ATGGACGAGGACTTTAAGAAGTTCGCCAACTTTCAAATGTACAACGGTGT |
| 1023 | TGATTCTTCCACCGAAGAAACCATTAAGAACTTGGCTAAGGATATCTCCGT |
| | TAACTCCGGTTTGAAGATCGAAAACTACACTACCTCCTCATCCAAAGAAGT |
| | CGAATCCATTTTGTCTGATTTGGAAGGTGTTGATCCAACTGCTAAAGACTT |
| | GCAAGATCAAAGATTGAACCCATCTTGTGACACCTTCTCTTCATCTTTGTG |
| | GGTTAAGAATTTGGCCGCCATTATTAACAACAACCCAGAGTATTACAAGC |
| | CCTACTCTTTGGATTGCATTTGGCAAAATTTGTCCGCTTACGGTGAAACCT |
| | CTGGTATTGAATCTCAAGCCAATTTGATTAACGCCCCACAAAAGATTATCA |
| | ACTCCGTTTACAGAACTGTCAAGCCAGTCAAGAAAGAAAAGTCCTTCACC |
| | ATCTTGAAAGAGATGGAAGGTTGTATTAACCCAGGTGAGTTGTTGGTTGT |
| | TTTGGGTAAACCAGGTTCTGGTTGTACTACTTTGTTGAAGTCCATTTCCAA |
| | CAACACCAACGGTTTCAAGATTACCAAAGACACCAAATTGTCTTACGCTG |
| | GTTTTACCCCAAGAGAAATGAAGGCTCATTATGGTGGTGAAGTTATCTAC |
| | AATGGTGAAATCGATGTCCACTTGCCAAAGTTGACTGTTTATCAAACCTTG |
| | TACACCGTCGCTAGATTGAAAACTCCACATAACAGAATTCAAGGTGTCGA |
| | CAGAGATATTTTCGCTAAGTACTTGACTGATGTTACCATGGCTACTTATGG |
| | TTTGTCTCATACCAGAGATACCATCATCGGTAACGATTTGATTAGAGGTGT |
| | TTCAGGTGGTGAAAGAAAGAGAGTTTCTATTGCTGAAACCGCTATTTGTG |
| | GTGCTAAATTGCAATGTTGGGATAACGCTACTAGAGGTTTGGATTCTGCT |
| | GCTGCTTTAGATTTCATTAGAGCCTTGAGAGTTAACGCCAATACCATGGG |
| | TGCTTCATCTGCTGTTGCTATCTATCATTGCTCTGAAGGTGCTTACTCTTT |
| | GTTCGATAAGGTCTGCATTTTGGATCAAGGTTACCAGTTGTTTTTCGGTCC |
| | AGCTAATGATGCTAAGCAATACTTTGAACAAATGGGTTACGTTTGCCCAG |
| | ATAGACAAACTACTGCTGATTTCTTGACCTCTGTTACTTCTCCAGAAGAAA |
| | GAGTTTTGAACAAGGACTTCTTGAAGAAGGGTATTGCCATTCCACAAACG |
| | CCTAAAGAAATGAACGAATACTGGCGTAAGTCTCAAGCTTACTCTGAATT |
| | GACTCACGAAATTCAAGCCAAGTTGTCCAAGAACGTTGAAGAATCTAGAA |
| | AGGTTATTAAGGCTGCTCATTTGGCTAAACAATCCGGTAAATGTAGAGAAT |
| | CTTCTCCATACACCGTCAACTACAGATTGCAGATCAAATACTTGCTGTCCA |
| | GAGAAATTTGGAGGATCACTAATTACCCAACCACCAACATTTTGATGTTGT |
| | TGGGTAACTCTGCTATGGCCTTGATTTTGGGTTCTATGTTCTACAAGATCA |
| | TGAAGAAGGATGACACCGAGTCTTTTTACTTTAGAGGTGCTGCTATGTTCT |
| | CTGCCACTTTGTTTAATGCTTTCAGCTGTTTGTTGGAGGTGTTCTCCTTGT |
| | ATGAATCCAGACCAATTATCGAAAAGCACAAGACCTACAGCTTGTATCATC |
| | CATCTGCTGATGCATTCGCCTCCATTATTTCTAGAGTTCCAGTTAAGATTG |
| | CCATTGCCATCTGTTTCAACGTCGTGTTCTACTTTTTGGTCCACTTTAAAA |
| | GAGATGCCGGCTCATTCTTCTTCTATTTCTTGATTAACCTGGTCACCGTGT |
| | TCTCCATGTCTCATATGTATAGAACCTTCGGTTCCATCACTAGATCTTTGC |
| | CAGAAGCTATGTTTCCAGCTGCTGTTTTGTTGTTGGCTATGGCTATGTATA |
| | CTGGTTTCGCTATTCCAAGAACTCAAATGTTAGGTTGGTCTACCTGGATTT |
| | GGTACATTAACCCATTGGCTTACTTGTTCGAATCCTTGATGGTTAACGAAT |
| | TCCACGGTAGAGAATTTGAATGCTCTACCTTTATTCCAATGGGCTCTATGT |
| | ACGCTAATTACACCGGTAACCATAGAATTTGTGCTGCTTTGGGTGCTATT |
| | GCTGGTGAAGATTATGTTTTAGGTGACGACTACATCAGAAACAACTACGG |
| | TTATGAACATTCCCATAAGTGGCGTGGTTTTGGTATTGGTATAGGTTACTC |
| | TGTGTTCTTCTTGATCACCTACTTGATCGTTTGCGAATTGAATCCTGGTAA |
| | GACTCAAAAGGGTGAGATTTTGATATACCCACAGCACATCATCAGAAGAT |
| | TGAGGAAGCAAAAAGAGTCTAAGGGTTCTACCGATTACAACTCTTCTGAC |
| | TTGGAAGCTGGTTCTTCTGATTCCTCTATTACCGATATTACCCTGCTGGAA |
| | GATCCAAAAGAATTGGAAGAAGAGTCTTTCAACTACACCGGTTTGTCTACT |
| | GCTAACAATGTTTTCCATTGGAGAAACGTTTGCTACGACATCGAAACTAAG |
| | AACGAGACTAAGAGGATCTTGAACAACATTGATGGTTGGGTTAAGCCAGG |
| | TACTTTGACTGCTTTGATGGGTGTTTCTGGTGCTGGTAAAACTACTTTATT |
| | GGATTGCTTGGCTGGTAGAGTTACCACCGGTATTATTTCTGGTGATATGT |
| | TCGTTAACGGTTCCTCCAGAGATTCATCTTTCCCAAGATCTATTGGTTACT |
| | GTCAACAACAGGACTTGCATTTGAGAACTTCTACTGTTAGGGAATCCCTG |
| | AGATTTTCTGCTTATTTGAGACAGCCAAAGTCCGTTCCAATTGCAGAAAAG |
| | AATGCCTATGTTGAAGAAGTCATCAGAACCTTGGACATGGAATTATATGCT |
| | GATGCTATCGTTGGTTCTCAAGGTGAAGGTTTGAATGTGGAACAGAGAAA |
| | AAGATTGACTGTCGGTGTTGAATTGGCTGCTAAACCTAAGTTGTTGATTTT |
| | CTTGGATGAACCCACTTCTGGTTTGGATAGTCAAACTGCTTGGTCTATTTG |
| | CCAGTTGATGAGAAAATTGGCCGACGAAAATCAAGCTATTTTGTGCACTA |
| | TTCATCAGCCAACAGCTATCTTGATGGAAAAGTTTGATAGGCTGTTGCTGT |
| | TGGAAAAAGGTGGTAAAACAGTTTACTTCGGTGAATTAGGTTCAGGTTGC |
| | AGAACCATGATTGACTACTTTGAAAGACATGGTGCTCCAAGATGTCCTAG |
| | AGATGCTAATCCAGCTGAATGGATGTTGGATATAGTTAGATCTCCAAAGA |
| | ACTCCGAATTGCAAGAGGATTACCATGATATCTGGCGTAATTCCATCGATT |
| | ACAAGTTGGTGCAAAAAGAACTGGACAAGTTGTCTAATGCTTCCACTGGT |
| | TACTCTACAAACACCGATTATGAAGATGATACCGAATTCGCTACCTCCTTG |
| | TTGTATCAAACTACTATTGTCGGTAAGAGGCTGATGGAACAATATTGGAG |
| | ATCACCACAATACCTGTGGTCTAAGTTTGCTTTGACCATCATCAACATGCT |
| | GTTCATCGGTTTTACTTTCTTCAGAACCTCTGCTAGCTTGCAAGGTTTACA |
| | ATCTCAAACTTTGGCCATCTTCATGTTCACCGTTATTTTCAACCCACTGCT |
| | GCAACAATATTTGCCAAACTATGCTCAACAGAGGGACTTGTACGAAGTTA |
| | GAGAAAGACCATCTAGAACGTTTTCATGGAAGGCTTTCATCATCTCCCAA |
| | ATCTTGATTGAGGTTTTCTGGAACTTCATTGCTGGTACTGTTGCTTTCGTT |
| | ATCTTCTACTACACCATCGGCTTTTACAGAAATGCTTCTTTGGCTAACCAG |
| | TTGTACGAAAGAGGTGTCTTGTTTTGGTTGTTATGTACCGCTTACTTCGTC |
| | TTTGTTGGTTCCATGGGTATTTTGACCATGTCCGCTATTGAAATTTTGGAA |
| | AACGCTGCCTACATTGCCTCATTGATGTTTACTATGTGTTTGTCCTTCTGC |
| | GGTGTTTTCACTACTAGAAAAGCTATGCCAAGATTCTGGATTTTCATGTAC |
| | AGAGTTTCTCCCTTGACCTACTTGTTGGAAGCTTTGTTGTCTGTTGGTGTT |
| | TCTAACGTTCCAATCAAGTGCAACGAAAACGAGTTCTTGAAATTGGTTCCA |
| | CCAACTGGTCAAACATGTGGTGATTATTTGTCTCCCTATTTGTTCGTTGCT |
| | CAAACTGGTTATTTGAAGGATCAATTGGCTACCGATGTTTGCTACTTGTGT |
| | GAATTGTCTCACACCAACGATTACTTGAAGAGAGTTGACGTTGATTACTCT |
| | CAAAGATGGCGTAACTACGGTATCTTCATCTCTTACATCGTGTTCAATTAC |
| | TGCGCCGGTATCTTGTTGTACTATATCTTTAGAGTCCCCAAGAAGAATCA |
| | GACCAAGATTAAGCCATCTAAGTAA |
| SEQ ID NO: | MDEDFKKFANFQMYNGVDSSTEETIKNLAKDISVNSGLKIENYTTSSSKEVESILSDLEGV |
| 1024 | DPTAKDLQDQRLNPSCDTFSSSLWVKNLAAIINNNPEYYKPYSLDCIWQNLSAYGETSGI |
| | ESQANLINAPQKIINSVYRTVKPVKKEKSFTILKEMEGCINPGELLVVLGKPGSGCTTLLKS |
| | ISNNTNGFKITKDTKLSYAGFTPREMKAHYGGEVIYNGEIDVHLPKLTVYQTLYTVARLK |
| | TPHNRIQGVDRDIFAKYLTDVTMATYGLSHTRDTIIGNDLIRGVSGGERKRVSIAETAICG |
| | AKLQCWDNATRGLDSAAALDFIRALRVNANTMGASSAVAIYHCSEGAYSLFDKVCILD |
| | QGYQLFFGPANDAKQYFEQMGYVCPDRQTTADFLTSVTSPEERVLNKDFLKKGIAIPQT |
| | PKEMNEYWRKSQAYSELTHEIQAKLSKNVEESRKVIKAAHLAKQSGKCRESSPYTVNYR |
| | LQIKYLLSREIWRITNYPTTNILMLLGNSAMALILGSMFYKIMKKDDTESFYFRGAAMFS |
| | ATLFNAFSCLLEVFSLYESRPIIEKHKTYSLYHPSADAFASIISRVPVKIAIAICFNVVFYFLVH |
| | FKRDAGSFFFYFLINLVTVFSMSHMYRTFGSITRSLPEAMFPAAVLLLAMAMYTGFAIPR |
| | TQMLGWSTWIWYINPLAYLFESLMVNEFHGREFECSTFIPMGSMYANYTGNHRICAA |
| | LGAIAGEDYVLGDDYIRNNYGYEHSHKWRGFGIGIGYSVFFLITYLIVCELNPGKTQKGEI |
| | LIYPQHIIRRLRKQKESKGSTDYNSSDLEAGSSDSSITDITLLEDPKELEEESFNYTGLSTAN |
| | NVFHWRNVCYDIETKNETKRILNNIDGWVKPGTLTALMGVSGAGKTTLLDCLAGRVTT |
| | GIISGDMFVNGSSRDSSFPRSIGYCQQQDLHLRTSTVRESLRFSAYLRQPKSVPIAEKNAY |
| | VEEVIRTLDMELYADAIVGSQGEGLNVEQRKRLTVGVELAAKPKLLIFLDEPTSGLDSQT |
| | AWSICQLMRKLADENQAILCTIHQPTAILMEKFDRLLLLEKGGKTVYFGELGSGCRTMID |
| | YFERHGAPRCPRDANPAEWMLDIVRSPKNSELQEDYHDIWRNSIDYKLVQKELDKLSN |
| | ASTGYSTNTDYEDDTEFATSLLYQTTIVGKRLMEQYWRSPQYLWSKFALTIINMLFIGFT |
| | FFRTSASLQGLQSQTLAIFMFTVIFNPLLQQYLPNYAQQRDLYEVRERPSRTFSWKAFIIS |
| | QILIEVFWNFIAGTVAFVIFYYTIGFYRNASLANQLYERGVLFWLLCTAYFVFVGSMGILT |
| | MSAIEILENAAYIASLMFTMCLSFCGVFTTRKAMPRFWIFMYRVSPLTYLLEALLSVGVS |
| | NVPIKCNENEFLKLVPPTGQTCGDYLSPYLFVAQTGYLKDQLATDVCYLCELSHINDYLK |
| | RVDVDYSQRWRNYGIFISYIVFNYCAGILLYYIFRVPKKNQTKIKPSK- |
| SEQ ID NO: | ATGTCCGAGAAGAAGGACATCATTGATTCTGGTGCTATGTCCCAAGTTGACGAATC |
| 1025 | TTCTTCTTTGAACTTGCAATCCTACGATGGTTTCGATCAAAACGCCCAAGATAAGATT |
| | AGACAATTGGCCAGATCTTTGACCAACCAGTCATCTACTACTCATAACAATGATGCT |
| | GCCAGCTTGTTCTCTAATGTTAAGGGTGTTAATCCCGTTTTCACCGATCCATCTAATC |
| | CAGAATATGACGAAAGATTGGATCCAGAGTCTGAGAACTTTTCTTCTACTGCTTGGG |
| | TTAAGAACATGGCTAATTTGACTGCTGCTGATCCAGATTACTACAAGCCATATTCTTT |
| | GGGTTGTGTTTGGAAAGATTTGACCGCTTCTGGTGATTCTTCTGATGTTGTTTATCA |
| | GTCCACCGTTTTCAACATGCCAACGAAGTTATTGAAAACCGCTTTCAGAAAAGCTAG |
| | GCCAGCTAAAGAATCTGATACCTTCCAAATTCTGAAGCCAATGGAAGGTTGTATTAA |
| | CCCAGGTGAATTATTGGTTGTTTTGGGTAGACCAGGTTCTGGTTGTACTACTTTGTT |
| | GAAGTCCATTTCCTCTAACACCCATGGTTTTAACGTTGGTAAGGATTCCACCATTTCT |
| | TACAATGGTTTGACTCCAAAGGCCATCAACAGACATTATAGAGGTGAAGTTGTTTAC |
| | AACGCCGAATCCGATGTTCATTTGCCACATTTGACTGTTTTCGAAACCTTGTACACTG |
| | TCGCCAGATTGAAAACACCATCTAATAGAGTTCAAGGTGTTGACAGAGATACTTAC |
| | GCTAAACATTTGACCGATGTTACTATGGCTACTTACGGTTTGTCTCATACCAGAAAT |
| | ACCAAGGTTGGTAACGATTTGGTTAGAGGTGTTTCTGGTGGTGAAAGAAAGAGAG |
| | TTTCTATTGCCGAAGTTACCATTTGCGGTTCTAAGTTTCAATGTTGGGATAACGCTAC |
| | TAGAGGTTTGGATTCTGCTACAGCTTTGGAATTCATTAGAGCCTTGAAAACTCAAGC |
| | TACCTTGACTAATACTGCTGCTACTATTGCTATCTACCAATGCTCTCAAGATGCCTAC |
| | GATTTGTTCGATAAGGTTTGTGTCTTGTACGGTGGTTACCAAATTTTCTATGGTTCTG |
| | CTCAAAAGGCTAAAAAGTACTTCGAAACTATGGGTTACCAGTGTCCAGAAAGACAA |
| | ACTACTGCTGATTTCTTGACTTCTGTTACTTCTCCAGCTGAAAGAGTTATCAACCCAG |
| | ATTTTATTGGTAGGGGTATCCAAGTTCCACAAACTCCAGAAGATATGAACAACTATT |
| | GGAGAAACTCCCCAGAGTACAAAGAATTGATCAACGAAATCGATACCCACTTGGCC |
| | AACAATCAAGATGAATCCAGAAACTCCATCAAAGAAGCTCATATTGCCAAGCAATCT |
| | AACAGAGCTAGACCTGGTTCTCCATACACTGTTAATTATGGTATGCAGGTCAAGTAC |
| | CTGTTGACTAGAAATGTTTGGAGGATCAAGAACAACTCCTCTGTCCAGTTGTTTATG |
| | ATTTTCGGTAATTGCGGTATGGCCTTCATTTTGGGTTCTATGTTTTACAAGGTTATGA |
| | AGCACGATTCCACCTCTACTTTTTACTACAGAGGTGCTGCTATGTTCTTCGCCATTTT |
| | GTTTAATGCTTTCTCGTGCTTGTTGGAGATCTTCTCATTATATGAAGCCAGACCAATC |
| | ACCGAAAAGCACAGATCTTATTCCTTGTATCATCCATCTGCTGATGCTTTCGCTTCCA |
| | TTTTCTCTGAAATTCCTACCAAGATCATTATCGCCATCGGTTTCAACATTATCTACTAT |
| | TTCTTGGTCAACTTCGAGAGAAATGGCGGTGTTTTCTTTTTCTACTGGCTGATTAACA |
| | TCGTTGCTGTTTTCGCTATGTCTCACTTGTTTAGAACTGTTGGTTCTCTGACTAAGAC |
| | CTTGTCTGAAGCTATGATTCCAGCATCTATGTTGTTGTTGGCTATGTCTATGTTTACC |
| | GGTTTCGCTATTCCAAAGACCAAAATGTTAGGTTGGTCTAAGTGGATCTGGTACATT |
| | AACCCAATTGCCTACTTGTTCGAGTCCTTGATGATTAACGAATTCCACGGTAGAAGA |
| | TTTGAATGCGCTGCTTTTATTCCATCTGGTCCAGCTTACTCTAACATTACTGCTACAG |
| | AAAGAGTTTGCGCTGTTTCAGGTTCTGTTGCTGGTCAATCTTATGTTTTAGGTGACG |
| | ATTACATCCGTGTCTCTTATGACTACTTGCACAAACATAAGTGGCGTGGTTTTGGTA |
| | TTGGTATGGCATACGCTATCTTTTTCTTGTTCGCTTACTTAGTTGTCTGCGAGTATAA |
| | TGAAGGTGCTAAGCAAAAAGGTGAGATGTTGGTTTTTCCACAATCCGTCTTGAGAA |
| | AGTTGAGAAAAGAAGGTCAGCTGAAGAAGGATTCCGAAGATATAGAAAACGGCTC |
| | TAACTCTTCTACCACTGAAAAACAGTTGTTGGAAGATTCCGATGAGGGTTCTTCTAA |
| | TGGTGATTCAACTGGTTTGGTTAAGTCCGAAGCTATTTTTCATTGGAGGAACTTGTG |
| | TTACGACGTCCAAATCAAAGACGAAACTAGAAGGATCTTGAACAACGTTGATGGTT |
| | GGGTAAAACCAGGTACTTTAACTGCTTTGATGGGTTCTTCAGGTGCTGGTAAAACTA |
| | CTTTATTGGACTGTTTGGCTGAAAGGGTTACAATGGGTGTAATTACTGGTGATGTTT |
| | TGGTTGATGGTAGACCAAGAGATGAATCTTTCCCAAGATCTATTGGTTACTGTCAAC |
| | AACAAGACCTGCACTTGAAAACCTCTACTGTTAGAGAATCCTTGAGATTCTCTGCTT |
| | ACTTGAGACAACCAGCTGAAGTTTCTGTTGAAGAAAAGGATGCTTACGTCGAAGAA |
| | GTCATTAAGATCTTGGAGATGGAAAAGTACGCTGATGCTGTTGTTGGTGTTGCCGG |
| | TGAAGGTTTAAATGTTGAACAAAGAAAGCGTTTGACCATCGGTGTTGAATTGGCTG |
| | CTAAACCTAAGTTGTTGGTCTTTTTGGATGAACCTACTTCTGGTTTGGATAGTCAAAC |
| | TGCTTGGTCTATTTGCCAGCTGATGAGAAAATTGGCTTCTCATGGTCAAGCTATCTT |
| | GTGCACTATTCATCAACCATCCGCTATTTTGATGCAAGAATTCGATAGGTTGCTGTTC |
| | TTGCAGAAAGGTGGTAAGACTGTTTACTTCGGTGAATTAGGTGAAGGCTGCCAAGT |
| | TATGATTGACTACTTTGAAAGAAACGGCTCCCATAAGTGTCCACCAGATGCTAATCC |
| | TGCTGAATGGATGTTGGAAGTTGTCGGTGCTGCTCCAGGTTCTCATGCTAATCAAG |
| | ATTATCATGAAGTCTGGCGTAACTCCGAAGAATTCAGAATAGTTCACGAAGAGTTG |
| | GACTTGATGGAAAGAGAATTACCAGCTAAATCTGCTGGTGTTGATACCGATCATCA |
| | AGAATTTGCTACTGGTTTGTTCTATCAGACCAAGTTGGTTTCCGTTAGGTTGTTCCAA |
| | CAGTATTGGAGATCACCTGAATACTTGTGGGCTAAGTTTGTGTTGACTATCTTCAAC |
| | GAGCTGTTCATCGGTTTTACTTTCTTTAAGGCTGGTACTTCATTGCAGGGCTTGCAA |
| | AATCAAATGTTGGCTGCTTTCATGTTCACCGTCATTTTTAACCCACTGTTGCAGCAAT |
| | ACTTGCCATCTTTTGTCCAACAAAGAGACTTGTACGAAGCTAGAGAAAGACCATCTA |
| | GAACATTTTCTTGGAAGGCTTTCATCGTGTCCCAAATATTGGTTGAAGCTCCATGGA |
| | ATTTCTTGGCAGGTACATTGGCTTACTTCATCTATTACTACCCAATCGGTTTCTACGA |
| | AAACGCTTCTTACGCAGGTCAATTGCACGAAAGAGGTGCTTTGTTCTGGTTATTTTC |
| | TACCGCTTTCTACGTTTACGTTGGTTCTATGGGTTTCTTGACCGTTTCCTTTAACGAA |
| | ATTGCTGAAAACGCTGCTAACTTGGCCTCTTTGATGTTTACAATGGCCTTGTCTTTTT |
| | GCGGTGTTATGACAACTCCATCTGCAATGCCAAGATTTTGGATCTTCATGTACAGAG |
| | TCTCTCCATTGACATACTTCGTTCAAGGTATTTTGGCTGTTGGTTTGGCTAACACCAA |
| | GATCGAATGTTCTTCATCTGAGTTCTTGCAATTTGAAGCACCATCAGGTATGACTTG |
| | TGGTAATTACATGGAAGCCTATTTGGATTATGCTGGTACTGGTTACTTGAAGGATGA |
| | ATCAGCTACTGGTACTTGTGAATTCTGCGAATACTCTTACACCAACGACTACTTGTCC |
| | TCCATTAACTCTTATTACTCTCAGAGATGGCGTAATTGGGGTATTTTCATTTGTTACA |
| | TTGCCATCAACTACATCGGTGGTATTGCTTTCTATTACTTGGCTAGAGTTCCCAAGAA |
| | ATCTAAGGTTGCTAAGAAGTAA |
| SEQ ID NO: | MSEKKDIIDSGAMSQVDESSSLNLQSYDGFDQNAQDKIRQLARSLTNQSSTTHNNDAA |
| 1026 | SLFSNVKGVNPVFTDPSNPEYDERLDPESENFSSTAWVKNMANLTAADPDYYKPYSLG |
| | CVWKDLTASGDSSDVVYQSTVFNMPTKLLKTAFRKARPAKESDTFQILKPMEGCINPG |
| | ELLVVLGRPGSGCTTLLKSISSNTHGFNVGKDSTISYNGLTPKAINRHYRGEVVYNAESD |
| | VHLPHLTVFETLYTVARLKTPSNRVQGVDRDTYAKHLTDVTMATYGLSHTRNTKVGND |
| | LVRGVSGGERKRVSIAEVTICGSKFQCWDNATRGLDSATALEFIRALKTQATLTNTAATI |
| | AIYQCSQDAYDLFDKVCVLYGGYQIFYGSAQKAKKYFETMGYQCPERQTTADFLTSVTS |
| | PAERVINPDFIGRGIQVPQTPEDMNNYWRNSPEYKELINEIDTHLANNQDESRNSIKEA |
| | HIAKQSNRARPGSPYTVNYGMQVKYLLTRNVWRIKNNSSVQLFMIFGNCGMAFILGS |
| | MFYKVMKHDSTSTFYYRGAAMFFAILFNAFSCLLEIFSLYEARPITEKHRSYSLYHPSADA |
| | FASIFSEIPTKIIIAIGFNIIYYFLVNFERNGGVFFFYWLINIVAVFAMSHLFRTVGSLTKTLSE |
| | AMIPASMLLLAMSMFTGFAIPKTKMLGWSKWIWYINPIAYLFESLMINEFHGRRFECA |
| | AFIPSGPAYSNITATERVCAVSGSVAGQSYVLGDDYIRVSYDYLHKHKWRGFGIGMAYA |
| | IFFLFAYLVVCEYNEGAKQKGEMLVFPQSVLRKLRKEGQLKKDSEDIENGSNSSTTEKQL |
| | LEDSDEGSSNGDSTGLVKSEAIFHWRNLCYDVQIKDETRRILNNVDGWVKPGTLTALM |
| | GSSGAGKTTLLDCLAERVTMGVITGDVLVDGRPRDESFPRSIGYCQQQDLHLKTSTVRE |
| | SLRFSAYLRQPAEVSVEEKDAYVEEVIKILEMEKYADAVVGVAGEGLNVEQRKRLTIGVE |
| | LAAKPKLLVFLDEPTSGLDSQTAWSICQLMRKLASHGQAILCTIHQPSAILMQEFDRLLF |
| | LQKGGKTVYFGELGEGCQVMIDYFERNGSHKCPPDANPAEWMLEVVGAAPGSHAN |
| | QDYHEVWRNSEEFRIVHEELDLMERELPAKSAGVDTDHQEFATGLFYQTKLVSVRLFQ |
| | QYWRSPEYLWAKFVLTIFNELFIGFTFFKAGTSLQGLQNQMLAAFMFTVIFNPLLQQYL |
| | PSFVQQRDLYEARERPSRTFSWKAFIVSQILVEAPWNFLAGTLAYFIYYYPIGFYENASYA |
| | GQLHERGALFWLFSTAFYVYVGSMGFLTVSFNEIAENAANLASLMFTMALSFCGVMTT |
| | PSAMPRFWIEMYRVSPLTYFVQGILAVGLANTKIECSSSEFLQFEAPSGMTCGNYMEAY |
| | LDYAGTGYLKDESATGTCEFCEYSYTNDYLSSINSYYSQRWRNWGIFICYIAINYIGGIAFY |
| | YLARVPKKSKVAKK |
| SEQ ID NO: | ATGGACAACGAATCCACCGATTCCTTGGTTGAATATCAAGGTTTCGATAACAAGGTC |
| 1027 | GAGAACCAGATTAGAGATTTGGCTAGAACTTTGTCCAGAGCCTCTTTGAAAGAACA |
| | AGGTTCTAACCACGATTTGGTTTCTGGTGCTGCTGATGATGATACCAGATCTATTTT |
| | CTCTACCAAGTACGAAGGTGTCAACCCAGTTTTCTCTGATGCTAATGCTCCAGGTTA |
| | TGATGCTAGATTGGATCCTAATTCCGACAACTTTTCTTCTGCTGCTTGGATCAGAAAT |
| | ATGGTTGCTTTGGCTGGTTGTGACCCAGAATATTACAAACATTACACCATTTCTTGCT |
| | GCTGGAAGGATTTGAGAGCTTTTGGTGATCCAACTGATGTTGCTTACCAATCTACTA |
| | TGGTTAACATGCCCCAAAAGATCTTCTCACAAGTCAAGAGATCTTTGTGCAAGCCAA |
| | AGGATGAACAAGTTTTCGATATCTTGAAGCCAATGGACGGTTTGTTGAAAGCTGGT |
| | GAATTATTGGTTGTTTTGGGTAGACCAGGTTCTGGTTGTTCTACCTTGTTGAAAACT |
| | ATTTCCGCTAACGTTCAAGGTTACCACTTGGACGAAAAATCCATCGTTTCTTACAAT |
| | GGTTTGGATGCTAAGACTATCGGTAAACACTATAGAGGTGAAGTTGTTTACAACGC |
| | CGAATCCGATGTTCATTTTCCACATTTGTCTGTCTTTGAAACCCTGTACAACATTGCT |
| | TTGTTGGTTACTCCATCCAACAGAGTTAAGGGTGTTTCTAGAGAAGATTTCGCTAAG |
| | CACGTTACTGAAGTTGCTATGGCTACTTATGGTTTGTCTCATACCAAAGATACCAAG |
| | GTCGGTAACGAATTGGTTAGAGGTGTTTCAGGTGGTGAAAGAAAGAGAGTTTCTAT |
| | TGCCGAAGTTACCATTTGGGGTTCTAGATTTCAATGTTGGGATAACGCTACTAGAGG |
| | TTTGGATTCTGCTACTGCTTTGGAATTCATTAGAGCCTTGAAAACCTCTACCGCTATT |
| | TCTGGTTCTACTGGTGTTATTGCTATCTACCAATGTTCCCAAGATGCCTACGATTTGT |
| | TCGATAAGGTTTGTGTTTTACACGAGGGCTATCAAATCTATTACGGTTCTGCTAAAG |
| | AAGCCAAGGGTTACTTCGAAAGAATGGGTTATGAATCCCCATCTAGACAAACTACT |
| | GCTGATTTCTTAACCGCTGTTACTAATCCAGCTGAAAGAATCCCAAATGAAGCCTTT |
| | GTCAAAGAAGGTCGTTACATTCCATCTACCGCAAAAGAAATGGAAGAATACTGGCG |
| | TAATTCTCCAGAATATGCTGCTTTGAGACAAGAAATTGAAGCCGAATTGTCTAAGG |
| | ATTCTACCGAAGCTAGACAAGAATTGCTAGATGCTCATGTTGCTAGACAATCCAAG |
| | AGACAAAGAAAGTCCTCTCCTTACATCGTTAACTTCGGTATGCAAGTTAAGTACTTG |
| | ACGATGAGAAACTTCCTGAGGATCAAAAAGTCTTACGGTATTACCGTTGGTACTATT |
| | GCTGGTAATACTGCTATGGCATTGGTTTTGGGTTCCATCTTTTACAAGTCTATGCAA |
| | GATACCACTACCGCCACATTTTTCTACAGAGGTGCTGCTATGTTTATCGCCGTTTTGT |
| | TTAATGCTTTCGCCTCCATGTTGGAGATCTTCAGCTTGTATGAAGCCAGACCAATTAT |
| | CGAAAAGCACAGAAGATACAGCTTGTACCATCCATCTGCTGATGCTTTAGCTTCTAT |
| | GTTGTCTGAATTGCCATCTAAGATTGTCACCGCCATTTGTTTCAACCTGATCTTGTAT |
| | TTCATGGTCAACTTCAGAAGAGAACCAGGTCCATTCTTCTTCTACTTTCTGATGAATT |
| | TCTTGGCCACCTTGGTTATGTCTGCTATTTTTAGATGTGTTGGTTCCGCTACTAAGAC |
| | CTTGTCTGAAGCTATGGTTCCAGCTTCTTGTTTGTTGTTGGCTATCTCATTATACGTC |
| | GGTTTCTCCATTCCAAAGAAGGACTTGTTAGGTTGGTCAAGATGGATTTGGTACATC |
| | AACCCATTGTCCTACATCTTCGAATCCTTGATGATCAATGAATTCGTCGGTAGAGAT |
| | TTCCCATGTGCTACTTTTGTTCCATCAGGTGCTGGTTATGAAGATATCGGTTCTTTAG |
| | AAAGAGTCTGCAATACTGTTGGTGCTGTTCCAGGTAATCCTAGAGTTTCTGGTTTGG |
| | CTTTCATTGAACAAACCTATGGTTACTCTGCTTCTCATAGATGGCGTTCTTTAGGTAT |
| | TGGTATTGCCTTCTTCATTTTCTTCACCGCCTTTTACTTGTTGTTCTGCGAATTCAATG |
| | AATCCGCCGTTCAAAAGGGTGAGATTTTGTTGTTTCCAAAGTCCGTCTTGAAGTCCA |
| | AAAGAAGGCAGTTGTCTAAGTCCAAGAACGATATTGAAACAGCCGATGATCCTGAA |
| | GGTGGTGTTACTGATCAAAAGTTGTTGCAAGACTCCTTGGAAGAGTCTAATGTCTCT |
| | TCATCTTCTGAAAAGTCTGCTAACGCTAATGTCGGTTTGTCTAAATCCGAAGCTATTT |
| | TCCATTGGAGAAACGTTTGTTACGACGTCCAGATTAAGAAAGAAACCCGTAGAATC |
| | TTGTCCAACGTTGATGGTTGGGTTGAACCAGGTACTTTGACTGCTTTGATGGGTTCT |
| | TCTGGTGCAGGTAAAACTACTTTGTTGGATTGCTTGGCATCTAGAGTTACCATGGGT |
| | GTTATTACTGGTGATATGTTCGTTAACGGTCACTTGAGAGATAACAGCTTTCCAAGA |
| | TCTATTGGTTACTGTCAACAACAGGACTTGCATTTGGCTACTGCTACTGTTAGAGAA |
| | TCTTTGAGATTCTCCGCTTACTTGAGACAATCCTCTGAAGTTTCAATCGAAGAGAAG |
| | AACTCCTACGTTGAAGATGTCTTGAGGATTTTGGAAATGGAACCTTACGCTGATGCT |
| | GTTGTTGGTATAGCCGGTGAAGGTTTGAATGTCGAACAAAGAAAAAGATTGACCAT |
| | CGGTGTTGAATTGGCTGCTAAACCTAAGTTGTTGTTGTTTTTGGATGAACCCACCTC |
| | CGGTTTAGATTCTCAAACTGCTTGGTCTATTTGCCAGTTGATGAGAAAATTGGCTGA |
| | TCACGGTCAAGCCATTTTGTGTACTATTCATCAACCATCCGCTCTGTTGATGCAAGA |
| | ATTTGATAGACTGCTGTTCTTGCAGAAAGGTGGTAAAACCATATACTTCGGTGACTT |
| | AGGTCCAGGTTGTGAAACTATGATCGACTACTTTGAATCTCATGGTGCTGATAAGTG |
| | TCCAGAAGGTGCTAATCCTGCAGAATGGATGTTGGAAGTTATTGGTGCTGCACCAG |
| | GTTCACATGCTAATCAAGATTATCATGAAGTCTGGCGTAACTCCGAAGAGTATAATG |
| | CTGTTCAACAAAAGTTGGACTGGATGGAAGTTGAGTTGGCTAAAAAGCCATTGGAC |
| | AACTCTTCAGAACAATCTGAATTCGGCACTTCCATTTTCTACCAGTGTAAGATAGTTA |
| | CCTTGAGGTTGTTCCAACAGTACTACAGAACTCCATCTTACATTTGGTCCAAGTTGTT |
| | CTTGACCATCTTCTCCCAATTATTCATCGGCTTCACCTTTTTCAAAGCCAAGTTGGAC |
| | ATGCAAGGTCTGCAAAATCAACTGTTTGCTGTTTTCACTTTCACCGTGATTTTTAACC |
| | CAGCTTGCCAACAATACTTGCCCTTGTTCGTTTCTCAAAGAGACTTGTACGAAGCAA |
| | GAGAAAGACCATCTAGAACATTTTCTTGGTTGGCCTTCATCTACTCCCAAATCGTTGT |
| | TGAAATTCCATTCAACGTTGTCTTGGGTACTATAGGCTTTTTCGTTTTCTATTACCCA |
| | ATCGGCTTTTACAACAACGCCTCTTATTCTGACCAATTGAACGAAAGAGGTGTCCTG |
| | TTTTGGTTATTCTCTGTTGCTTTCTACGTGTTCATCTCCTCTATGGGTCAATTGTGTAT |
| | TGCAGGTTTGGAATACGCTGAAGCTGCAGGTAATTTGGCATCTTTGTGTTTCACTAT |
| | GTCCTTGAATTTCTGCGGTGTTTTTGGTGGTCCAGGTGTTTTGCCAGGTTTTTGGGTT |
| | TTTATGTACAGAGTCTCTCCCCTGTCTTACTTCATTGATGGTGTTTTGTCTACAGCCTT |
| | GGCTAACAATCCAGTTACTTGTGCTGATTACGAGTACTTGTCCTTTGTTCCTAAATCT |
| | GGTGAAACTTGTGGTGAGTACATGTCTACTTACATTGCTACTTACGGTGGTTACATC |
| | TTAGATCCAGATGCTACAGATGAATGCTCCTTCTGTAGAATTTCTTCTACCAACGCTT |
| | TCCTGTCCTCTTTCCAATCTTCTTATCATAGACGTTGGAGGAACTTCGGAATCTTCAT |
| | CGTTTTTATCGTTTTCAATTGGGCCGGTTGCATCTTCTTTTACTGGTTGGCTAGAGTT |
| | CCTAAGAAGAACAATAGAGTTGCCAACGAAAGAAATCCAGATAGAGAAACCACCA |
| | AGCAAATTTCTACTCATGGTGAAAAGTCCAAGCCACAACAAATTGAACAAGTCTAA |
| SEQ ID NO: | MDNESTDSLVEYQGFDNKVENQIRDLARTLSRASLKEQGSNHDLVSGAADDDTRSIFST |
| 1028 | KYEGVNPVFSDANAPGYDARLDPNSDNFSSAAWIRNMVALAGCDPEYYKHYTISCCW |
| | KDLRAFGDPTDVAYQSTMVNMPQKIFSQVKRSLCKPKDEQVFDILKPMDGLLKAGELL |
| | VVLGRPGSGCSTLLKTISANVQGYHLDEKSIVSYNGLDAKTIGKHYRGEVVYNAESDVHF |
| | PHLSVFETLYNIALLVTPSNRVKGVSREDFAKHVTEVAMATYGLSHTKDTKVGNELVRG |
| | VSGGERKRVSIAEVTICGSRFQCWDNATRGLDSATALEFIRALKTSTAISGSTGVIAIYQC |
| | SQDAYDLFDKVCVLHEGYQIYYGSAKEAKGYFERMGYESPSRQTTADFLTAVTNPAERI |
| | PNEAFVKEGRYIPSTAKEMEEYWRNSPEYAALRQEIEAELSKDSTEARQELLDAHVARQ |
| | SKRQRKSSPYIVNFGMQVKYLTMRNFLRIKKSYGITVGTIAGNTAMALVLGSIFYKSMQ |
| | DTTTATFFYRGAAMFIAVLFNAFASMLEIFSLYEARPIIEKHRRYSLYHPSADALASMLSEL |
| | PSKIVTAICFNLILYFMVNFRREPGPFFFYFLMNFLATLVMSAIFRCVGSATKTLSEAMVP |
| | ASCLLLAISLYVGFSIPKKDLLGWSRWIWYINPLSYIFESLMINEFVGRDFPCATFVPSGA |
| | GYEDIGSLERVCNTVGAVPGNPRVSGLAFIEQTYGYSASHRWRSLGIGIAFFIFFTAFYLL |
| | FCEFNESAVQKGEILLFPKSVLKSKRRQLSKSKNDIETADDPEGGVTDQKLLQDSLEESN |
| | VSSSSEKSANANVGLSKSEAIFHWRNVCYDVQIKKETRRILSNVDGWVEPGTLTALMGS |
| | SGAGKTTLLDCLASRVTMGVITGDMFVNGHLRDNSFPRSIGYCQQQDLHLATATVRES |
| | LRFSAYLRQSSEVSIEEKNSYVEDVLRILEMEPYADAVVGIAGEGLNVEQRKRLTIGVELA |
| | AKPKLLLFLDEPTSGLDSQTAWSICQLMRKLADHGQAILCTIHQPSALLMQEFDRLLFLQ |
| | KGGKTIYFGDLGPGCETMIDYFESHGADKCPEGANPAEWMLEVIGAAPGSHANQDYH |
| | EVWRNSEEYNAVQQKLDWMEVELAKKPLDNSSEQSEFGTSIFYQCKIVTLRLFQQYYR |
| | TPSYIWSKLFLTIFSQLFIGFTFFKAKLDMQGLQNQLFAVFTFTVIFNPACQQYLPLFVSQ |
| | RDLYEARERPSRTFSWLAFIYSQIVVEIPFNVVLGTIGFFVFYYPIGFYNNASYSDQLNERG |
| | VLFWLFSVAFYVFISSMGQLCIAGLEYAEAAGNLASLCFTMSLNFCGVFGGPGVLPGFW |
| | VFMYRVSPLSYFIDGVLSTALANNPVTCADYEYLSFVPKSGETCGEYMSTYIATYGGYILD |
| | PDATDECSFCRISSTNAFLSSFQSSYHRRWRNFGIFIVFIVFNWAGCIFFYWLARVPKKN |
| | NRVANERNPDRETTKQISTHGEKSKPQQIEQV |
| SEQ ID NO: | ATGTCCCAAGAACACTCCGACCAATCATTATATGATGGCCCAAACAAGAAAGAGAT |
| 1029 | CAGAGATTTGGCTAGAACTTTGACTGCTGCTTCTGTTGCTGTTTCTGGTAATTCTGAT |
| | GCTGCTGTTAATCCATTGGCTGCTCAACCAGGTGAACCAGGTTATAATGCTAGATTG |
| | GATCCTAACTCCGACGAATTTTCTTCAGAAGCTTGGGTTAGAAACTTGTCTCATTTG |
| | ACAGCTGAAAACCCAGATTACTACAAGCCATTTTCTTTGGGTTGTGCTTGGAAGAAT |
| | TTGAGAGCTTATGGTGATTCTACCGATGTTGCTTACCAATCTACTGTTGCTAATTTGC |
| | CATGGAAGTTGTTGCAATTCGGTTACAGATGTGTCAGAAAGTCTAGACCATCTGAT |
| | AAGTTCGACATCTTGAAGTCCATGGATGGTATTTTGAACCCAGGTGAATTATTGGTT |
| | GTTTTGGGTAGACCAGGTTCTGGTTGTTCTACTTTGTTGAAGTCTATCTCCTCTAACA |
| | CCCATGGTTTTAAAGTTGCTCCAGAATCCGAAATCAGATACGATGGTTTGACCCCAA |
| | AAGAAATTGCTAAACACTACAGAGGTCAAGTTGTTTACAACGCCGAATCTGATGTTC |
| | ATTTCCCACATTTGTCTGTTTTCGACACCTTAAAGACCATTGCTTTGTTGTCTACTCCA |
| | GCCAATAGAATCGAAGGTATGGATAGAGAAACCTACGCTAAACATGTTACCGAAGT |
| | TTACATGGCTACTTACGGTTTGTCTCATACCAGAAATACCAAGGTTGGTAACGATTT |
| | GGTTAGAGGTGTTTCAGGTGGTGAAAGAAAGAGGGTTTCTATTGCTGAAGTTGCTA |
| | TTTGCGGTTCTAAGTTGCAATGTTGGGATAATGCTACTAGAGGTTTGGATTCTGCTA |
| | CTGCTTTGGAATTCATTAAGGCCTTGAGAGTTAATGCCCAAATGACTAATACCTCCG |
| | GTGTTATTGCTATCTACCAATGTTCTCAAGATGCCTTCGATTTGTTCGATAAGGTTTG |
| | TGTTTTACACGAGGGTTATCAGATCTATTATGGTCCAGCATCTGAAGCCAAGCAATA |
| | CTTTGAAGATATGGGTTACGTTTCCCCAGAAAGACAAACTACTGCTGATTTCTTAAC |
| | CGCTGTTACTAATCCAGCCGAAAGGATTATGAATCAAGAGTTCATCAAGCAGAACA |
| | AGTTCATTCCAAGAACCGCTGAAGAGATGGAAAAACATTGGAGAAACTCCTCCAAC |
| | TACAAGAGATTGATTGGTCAAATCGATGAATGCTTCGCTAGAGATTCTGATAAGGC |
| | TAAGCAAGAATTGCAAGATGCTCATACTGCTAAGCAGTCTAAAAGATCAAGACCAT |
| | CTTCTCCATACACCGTTTCTTATGGTATGCAGGTTAAGTACCTGCTGAGAAGAAACA |
| | TCCAAAGAATTAGAAGTGATGCCGGTGTTACCATCTTCCAAGTTGTTGGTAATGCTG |
| | CTATGGCTTTCATTTTGGGTTCTATGTTCTACAAAATCCTGAAGCACGATGATACCGC |
| | TGGTTTTTATTCTAGATCTGGTGCTTTGTTCTTCGCCGTTTTGTTTAATGCTTTCTCCT |
| | GCTTGTTGGAAATCTTCGCATTATACGAAGCCAGACCAATTTCTGAAAAGCACAAG |
| | AGATACAGCTTGTACCATCCTTCTGCTGATGCTTTAGCTTCCGTTATTAGTGAAATTC |
| | CAGCTAAGATCGTTACCGCCATTTGTTTTAACATTGCCCTGTACTTTTTGTGCAACTT |
| | GAGACAATCTGCTGGTGCCTTTTTCTTTTACTTCCTGATGAATATGGTTGCCACCTTT |
| | GCTATGTCCCATATTTTCAGATGTTTGGGTGCTGCTACTAAGACTTTTTCTGAAGCTA |
| | TGGTTCCCTCCTCTTTGTTGTTGTTATCTATGGCTATCTACACCGGTTTCGCTATTCCA |
| | AAGACAAAAATGTTAGGTTGGTCCAAGTGGATCTCCTACATTGATCCATTGTCCTAC |
| | ATCTTCGAATCCTTGATGGTTAACGAATTCCACGGTAGAAAGTTCCAATGCTCTGTT |
| | TATGTTCCAACTGGTCCAGCTTATGCTAATGCTACAGGTACTGAAAGAGTTTGTTCA |
| | GCTGTTGGTGCTGTTCCAGGTCAAGATTATGTTTTAGGTGATGACTACCTGAGGCTG |
| | TCTTACAATTACTTGCACAAACATAAGTGGCGTGGTTTCGGTATTGGTATGGCTTAT |
| | GTTGTTTTCTTCTTGGGTGTTTACTTGGCCTTCACTGAATTCAACGAATCTGCTAGAC |
| | AAAGAGGTGAAGTTTTGGTTTTCACCAGGGAATCTTTGAAGAAGATGAAGAGAGCT |
| | AAGAAGTTGGAATCAGCTAGAGGTGATGCTGAAAATTCTGCTGGTATGGAAACTG |
| | GTATCAACGAGAAGAAGTTGCTTGAAGAATCTGGTGAATCTTCCACCTCATCTTTCC |
| | AAGATGTTAAGTTGTCTCAAACCGAGGCTATTTTTCATTGGAGGAACGTTTGTTTCG |
| | ACGTGAAGATTAAGAAAGAAGATAGGCGTATCTTGAACAACGTTGATGGTTGGGTT |
| | AAGCCAGGTACTTTAACTGCTTTGATGGGTTCTTCTGGTGCTGGTAAAACTACTTTA |
| | TTGGACTGTTTGGCTTCTAGAGTTACTACTGGTGTTGTTACTGGTGATATGTTCGTTA |
| | ACGGTCATTTGAGAGATGCTTCATTCGCTAGATCTATTGGTTACTGTCAACAACAAG |
| | ACTTGCACTTGCAAACTGCTACTGTTAGAGAATCATTGAGATTCGCTGCTTATCTAA |
| | GACAACCAGCTTCTGTTTCTACCGAAGAAAAGAACGATTACGTCGAAGAAATCATC |
| | AGGATCTTGGACATGGAAAAGTACGCTGATGCCGTTGTTGGTGTTGCTGGTGAAGG |
| | TTTGAATGTTGAACAGAGAAAAAGATTGACCATCGGTGTTGAATTGTCTGCTAAAC |
| | CTAAGCTGCTGTTGTTTTTGGATGAACCTACTTCTGGTTTGGACTCTCAAACTGCTTG |
| | GGCTATTTGTCAATTGATGAGAAAGTTGGCTAACCATGGTCAAGCTATTTTGTGCAC |
| | TATTCATCAACCATCCGCTTTGTTGATGCAAGAATTCGATAGACTGCTGTTCTTGAAA |
| | AGAGGTGGTAGAACTGTTTACTTCGGTGATCTAGGTGATGGTTGCTCTAAGATGAT |
| | TGACTACTTTGAATCTCAAGGTGCTCCAAAATGTCCACCAGGTGCTAATCCTGCTGA |
| | ATGGATGTTGGAAGTTATTGGTGCTGCTCCTGGTTCTCATACAGATAAGGATTACG |
| | GCGAAGTTTGGAGAAATTCAGATGAGTATAGAGCCGTTCAAGAGGAATTGGATTG |
| | GATGGAAAGAGAATTGCCAAAAAGACCTTTGGATACTGCTGCAGAACAAACTGAAT |
| | TTGCCACTTCTTTGTTCACCCAGTACAAGTTGGTTACTCAAAGGTTGTTCCAACAGTA |
| | TTGGAGAACACCATCTTACTTGTGGTCCAAGATTATCCTGACCTTGATTTCCCAAATC |
| | TTCATCGGTTTCACGTTCTTCAAGTCCGATTCTACATTGCAAGGTCTGCAAAATCAGA |
| | TGTTGTCCATTTTCATGTTCACCGTCGTTTTCAATCCAACCTTGCAACAATACTTGCCA |
| | TCCTTTGTTTCTCAGAGAGACTTGTACGAAGCTAGAGAAAGACCATCCAGAACATTT |
| | TCTTGGAAGGCTTTCATCTTGTCCCAAATTACTGTTGAAGCCCCATGGAATTTTGCTG |
| | TTGGTACTTTGGGTTTCCTGATCTATTACTACCCAGTGTCTTTTTACAGAAACGCTTC |
| | TTACGCACATCAGTTGCACGAAAGAGGTGCTTTATTTTGGTTATTCTGTACCGCCTTC |
| | TACGTTTACGTTGGTTCTATGGGTCAATTGTGCATTGCTGGTATTGAAGTTGCAGAA |
| | TCTGCAGGTCATATTGCCTCATTGATGTTTACCTTGTCCTTGTCTTTTTGCGGTGTTAT |
| | GGTTACTCCACAAAACATGCCAGGTTTTTGGAAGTTTATGTACAGAGCTTCTCCACT |
| | GACCTACTTCATAGATGGTTTAATGTCTACTGGTTTGGCTAATGCTCCAGCTCATTGC |
| | TCTCATTATGAATTGGTTAGTTTTACTCCACCAGCCGGTCAAACTTGTGGTGAGTAT |
| | ATGGCTCCATATATCAAAATGGCTGGTACTGGTTATTTGACCTCTCCATCAGCTACT |
| | GATAAGTGTTCTTTTTGTCCAGTCTCTACTACCAACGATTACTTGGCTCAAGTTTCCT |
| | CACATTACAAAGATAGATGGCGTAACTGGGGTATTTTCATCTGCTACATTTTCATCG |
| | ACTTTGGCTTCGCAATCTTCTTTTATTGGCTTGCAAGAGTCCCAAAGAAGAAAAACA |
| | GAGTTGCTGATGAACGTGATCCAGATGCTCCAAAGAAATCTGTTGCAGGTACTAAG |
| | AACTAA |
| SEQ ID NO: | MSQEHSDQSLYDGPNKKEIRDLARTLTAASVAVSGNSDAAVNPLAAQPGEPGYNARL |
| 1030 | DPNSDEFSSEAWVRNLSHLTAENPDYYKPFSLGCAWKNLRAYGDSTDVAYQSTVANLP |
| | WKLLQFGYRCVRKSRPSDKFDILKSMDGILNPGELLVVLGRPGSGCSTLLKSISSNTHGF |
| | KVAPESEIRYDGLTPKEIAKHYRGQVVYNAESDVHFPHLSVFDTLKTIALLSTPANRIEGM |
| | DRETYAKHVTEVYMATYGLSHTRNTKVGNDLVRGVSGGERKRVSIAEVAICGSKLQCW |
| | DNATRGLDSATALEFIKALRVNAQMTNTSGVIAIYQCSQDAFDLFDKVCVLHEGYQIYY |
| | GPASEAKQYFEDMGYVSPERQTTADELTAVTNPAERIMNQEFIKQNKFIPRTAEEMEK |
| | HWRNSSNYKRLIGQIDECFARDSDKAKQELQDAHTAKQSKRSRPSSPYTVSYGMQVKY |
| | LLRRNIQRIRSDAGVTIFQVVGNAAMAFILGSMFYKILKHDDTAGFYSRSGALFFAVLFN |
| | AFSCLLEIFALYEARPISEKHKRYSLYHPSADALASVISEIPAKIVTAICFNIALYFLCNLRQSA |
| | GAFFFYFLMNMVATFAMSHIFRCLGAATKTFSEAMVPSSLLLLSMAIYTGFAIPKTKML |
| | GWSKWISYIDPLSYIFESLMVNEFHGRKFQCSVYVPTGPAYANATGTERVCSAVGAVP |
| | GQDYVLGDDYLRLSYNYLHKHKWRGFGIGMAYVVFFLGVYLAFTEFNESARQRGEVLV |
| | FTRESLKKMKRAKKLESARGDAENSAGMETGINEKKLLEESGESSTSSFQDVKLSQTEAI |
| | FHWRNVCFDVKIKKEDRRILNNVDGWVKPGTLTALMGSSGAGKTTLLDCLASRVTTGV |
| | VTGDMFVNGHLRDASFARSIGYCQQQDLHLQTATVRESLRFAAYLRQPASVSTEEKND |
| | YVEEIIRILDMEKYADAVVGVAGEGLNVEQRKRLTIGVELSAKPKLLLFLDEPTSGLDSQT |
| | AWAICQLMRKLANHGQAILCTIHQPSALLMQEFDRLLFLKRGGRTVYFGDLGDGCSK |
| | MIDYFESQGAPKCPPGANPAEWMLEVIGAAPGSHTDKDYGEVWRNSDEYRAVQEEL |
| | DWMERELPKRPLDTAAEQTEFATSLFTQYKLVTQRLFQQYWRTPSYLWSKIILTLISQIFI |
| | GFTFFKSDSTLQGLQNQMLSIFMFTVVFNPTLQQYLPSFVSQRDLYEARERPSRTFSWK |
| | AFILSQITVEAPWNFAVGTLGFLIYYYPVSFYRNASYAHQLHERGALFWLFCTAFYVYVG |
| | SMGQLCIAGIEVAESAGHIASLMFTLSLSFCGVMVTPQNMPGFWKFMYRASPLTYFID |
| | GLMSTGLANAPAHCSHYELVSFTPPAGQTCGEYMAPYIKMAGTGYLTSPSATDKCSFC |
| | PVSTTNDYLAQVSSHYKDRWRNWGIFICYIFIDFGFAIFFYWLARVPKKKNRVADERDP |
| | DAPKKSVAGTKN |
| SEQ ID NO: | ATGTCCGAACACAGATTGCAGAGAAGATTATTGACTCCATTCCTGTCTAAAAAGGTC |
| 1031 | CCTCCAATTCCTGAAGAAGATGAGAGAATCGTTTACCCAAAAAGGCCAAACATCTTC |
| | TCCATCATGTTTTTCTGGTGGTTGGGTCCAGTTATGAGAGCTGGTTACAAAAGAACT |
| | TTGACTGCTCAGGACCTGTACAAGTTGAACGATGATATTAAGGTCGAGTCCATGAC |
| | CAGAAGATTCGAACAATACTTCCAGAACTCTTTGAACAAAGCTATGGCTGCTCATGT |
| | TAGAGCTAAAATGAAGGCTAGAGGTGAAACTGCTGAAACTAACACTGTTGATTTCG |
| | AGAAGGACATGGAAGATTTCGAAGTTCCCAAGATGATTATCGTTAGAGCCATTGCT |
| | AGAACCTTCACTTTTCAATATGGTATGGCTTGCTTGATGATGGCTTTGGGTTCTACT |
| | GGTAATTCTACTACTCCTTTGTTGACCAAGAAGTTGATCAGATTCGTTGAATTGAGA |
| | ACCTTGGGTCTAGAAAAGCACATGAATGCTGGTATTGGTTACGCTATTGGTTCCTCC |
| | ATTATGGTTTTGGTTAACGGTTTGTTCGTTAACCATGCCTTCCATTTGTCTATGTTGA |
| | CTGGTGCTCAAGTTAAGGCTGCTTTGACAAAAGCTTTGTTGGACAAGTCTTTCAGAT |
| | TGTCCGATGCCTCTAAACATGAATTCCCAACTTCTAAGATCACCTCTATGATGGGTG |
| | CTGATTTGGCTAGAATTGATTTTGCCTTGGGTTTCCAACCATTCTTGATTACTTTTCC |
| | AGTTCCAATCGCCATCTCCATTGGTATTTTGTGTCATAATATTGGTGCTCCAGCTATG |
| | GTTGGTGTTGGTTTGATTATTGTGTTCATGGTCTTGACCATGTTCGTCACTGGTAAG |
| | TTGTTTGCTTACAGAAAGACTGCTAACGTTTTCACCGATGCTAGAGTTAACTACGTC |
| | AAAGAAGTCCTGAACAACTTGAAGGTCATCAAGTTTTACTCTTGGGAGATCCCATAC |
| | TCCAAGATTATTACCGAGAACCGTAACAAAGAGATGAACATCGTTTACATCATGCAA |
| | GTCGCCAGAAACATTATTACCTCATTGGGTATGTCTCTGACCCTGTTTTCTTCTTTGG |
| | TTGCATTTTTGGTCTTGTACGCCATTCAAGGTTCTACAAAAGATCCAGCCTCTATCTT |
| | CTCCTCGATTTCTTTGTTTTCTGCCTTGGCTTCTCAGGTTTTCATGATTCCATTGGCTT |
| | TGGCTTCAGGTTCTGATGCTTTGATTGGTGTTACTAGGATTGGTGAATTTTTGGCTG |
| | CTGAAGAAGTTAACCCAAGATCCGCTAGAATTACTGCTTCTGAAAAGACTAAGGCT |
| | GATATGGAAGCTAACAAGTTGGCCTTGTCTATTAAGAACGCTTCTTTCAAGTGGGA |
| | AACCTTTGGTGAACAAGGTTCCCATACAAACAACACTCATACCTTGTCCGAAAAAGA |
| | AAACTTCACCGCTTCAGAGAAAGAGCTGTACAACAAATTGGAAAAACACATGACCA |
| | ACAAGTCCATCTCCACCATTTCTCATGAATCTGCTTCATCTCCAGATCATGGTCCTTTT |
| | TCTGATTCTTCTTCATTGGCCGAACAAAATTTCCCAGGTTTGGAATCCATTGATTTGG |
| | ATGTTAAGAAGGGTGAATTCGTTGTTATCACTGGTTTGATCGGTTCTGGTAAAACCT |
| | CTTTGTTGAATGCTATGGCCGGTTTCATGAAGAGAGTTTCTGGTTCTGTTGATGTCA |
| | ACGGTTCTTTGTTGTTGTGTGATCAACCATGGATTCAAAACGCTACCGTTAGAGAAA |
| | ACATCTTGTTTGGTTTGCCAATGGATGATGCCAAGTACAAGAATGTTGTTTACGCTT |
| | GCTCTTTGGAATCTGACTTGGAAATTTTACCTGCTGGTGATTTGACCGAAATTGGTG |
| | AAAGAGGTATTACTTTGTCTGGTGGTCAAAAGGCTAGAATCAATTTGGCAAGAGCT |
| | GTTTATGCCGATATGGACATTATCTTGTTGGATGATGTTTTGTCCGCTGTTGATGCA |
| | AGAGTTGGTAAACATATTATGGCCCAATGCATCATGGGTATCTTGAAGGATAAGAC |
| | CAGAATTTTGGCTACCCACCAATTGTCATTGATTGGTTCTGCTGATAGGATCGTTTTC |
| | TTGAATGGTAACGGTTCCATTTCCGTTGGTACTTTCGAAGAATTGAAGTCCAAGAAC |
| | GAAGCCTTCTCTCATTTGATGGCCTTTAATGCTGATTCCCATGATTCTGAGGATGAG |
| | AAAGAAGAAGAAGTCGAGGACGAGGAAGAAAGAGAAGCTGTAAAAGAATTGGTT |
| | GAAAGGCAAGTCACCAGATACAAATCTAGAGCTGACGAAGAAGAAATTAGGCACG |
| | ATTATGAAGCTAACGCTGATAAGGATGGTAGATTGATCGATGATGAAGAAAAGGC |
| | TGAAAACGGTATCAAGTTCGAAGTCTACAAGAACTACTTGTCCATTGGTTCCGGTAT |
| | TTTCAAGCACTATTCATCCGTTCCTATCGTCATCGTTTTGATTGCTTTGTCTGTTTTCT |
| | GTCAGCTGTTCACTAATACCTGGTTGTCTTTCTGGTCCGAAAAGAAGTTTGCTCATA |
| | AGTCTAACGGTTTCTACATCGGCTTTTACGTTATGTTCACATTCTTGGCTGCTATTTTC |
| | TTGACCTTGGAATTCGTTTTGTTGGCCTACTTGACTAACAACGCTTCTACTAAGTTGA |
| | ACTTGATGGCTGTTCAAAAGGTTTTGAGAGCCCCAATGTCTTACATGGATGTTACTC |
| | CAATGGGTAGAATCTTGAACAGATTCACTAAGGATACCGATACCTTGGATAACGAA |
| | ATCGGTAACCAGATTAGAATGCTGACCTACTTCTTCTCTAACATCGTTGGTGTTTTGG |
| | TTTTGTGCGTTATCTACTTGCCATGGTTTGCTATTGCTATTCCATTCTTGGGTTTTATC |
| | TTCGTTGCCATCGGTAATTTCTATCAAGCTTCTGCTAGAGAGATCAAGAGATTGGAA |
| | GCAGTTCAAAGATCCTTCGTCTACAACAACTTCAACGAAACATTGTCTGGTATGAAG |
| | ACCATTAAGGCTTACCAAGCTGAAGAAAGGTTCTTGGAAAAGAACGATACCTACGT |
| | CAACAACATGAACGAAGCTTACTACATCACCATTGCTAATCAGAGATGGTTGTCCAT |
| | CCATTTGGATTTTGTTGCTGCTGGTTTCGCCTTGATTATTTGCTTTTTGTGTGTGTTCA |
| | GGGTTTTCAAGATTTCCGCTGCTTCAGTTGGTCTGTTGTTGTCTTACGTTTTACAAAT |
| | TGCCGGTCAGTTGTCCATGTTGGTTAGAACTTATACCCAAGTCGAAAACGAGATGA |
| | ACTCCGTTGAAAGAATTTGTGAATACGCCTTGTATTTGCCAGAAGAAGCTCCATACA |
| | ACATCTCAGAAACAAAACCAGCTGATTCTTGGCCAGAACATGGTGCTGTTAGATTC |
| | GAAAATGTTTCCTTGGCTTATAGACCAGGTTTGCCATTGGTTTTGAAAAACTTGTCT |
| | GCCGACATTAAGCCCAAAGAAAAGATTGGTATATGTGGTAGAACTGGTGCCGGTA |
| | AATCTTCTATTATGACTGCCTTGTACCGTCTGTCTGAATTGAATGGTGGTAAGATTG |
| | AAATCGATGGTGTCGACATTTCTAAGTTGGGTTTGAACGATTTGAGGTCCAAGTTGT |
| | CTATCATTCCACAAGATCCAGTTTTGTTCAGGGGTACTATTAGAAAGAACTTGGATC |
| | CCTTCAACTTGAGTTCTGAAGATGTATTGTGGTCTGCTTTGCGTAGAGCAGGTTTGA |
| | TTGAAGAATCCAAGTTGGAATTTGTCAAGACCCAAAAACCAGATGCTGAAAACTTG |
| | CATAAGTTCCACTTGGATAGAGAAGTCGAAGATGAAGGTTCCAACTTTTCATTGGG |
| | TGAAAGACAGTTGATTTCCTTCGCTAGAGCAATGGTTAGAGACTCTAAGATCTTGAT |
| | TTTGGATGAAGCCACTTCCTCCGTTGATTACGAAACTGATTCTAAGATTCAGTCCAC |
| | CATCGTCAGAGAATTTGGTAACTGTACCATTTTGTGCATTGCCCACAGATTGAGAAC |
| | TATCTTGAACTACGATAGGATCTTGGTATTGGATAGGGGTGAGATCAAAGAATTCG |
| | ATACTCCATGGAACTTGTTCCAGTCAGAAGATGGTATCTTTAAGCAGATGTGCGAA |
| | AGATCCAACATCACCAAGGATGATTTTAAGCACTGA |
| SEQ ID NO: | MSEHRLQRRLLTPFLSKKVPPIPEEDERIVYPKRPNIFSIMFFWWLGPVMRAGYKRTLTA |
| 1032 | QDLYKLNDDIKVESMTRRFEQYFQNSLNKAMAAHVRAKMKARGETAETNTVDFEKD |
| | MEDFEVPKMIIVRAIARTFTFQYGMACLMMALGSTGNSTTPLLTKKLIRFVELRTLGLEK |
| | HMNAGIGYAIGSSIMVLVNGLFVNHAFHLSMLTGAQVKAALTKALLDKSFRLSDASKH |
| | EFPTSKITSMMGADLARIDFALGFQPFLITFPVPIAISIGILCHNIGAPAMVGVGLIIVFMV |
| | LTMFVTGKLFAYRKTANVFTDARVNYVKEVLNNLKVIKFYSWEIPYSKIITENRNKEMNI |
| | VYIMQVARNIITSLGMSLTLFSSLVAFLVLYAIQGSTKDPASIFSSISLFSALASQVFMIPLA |
| | LASGSDALIGVTRIGEFLAAEEVNPRSARITASEKTKADMEANKLALSIKNASFKWETFGE |
| | QGSHTNNTHTLSEKENFTASEKELYNKLEKHMTNKSISTISHESASSPDHGPFSDSSSLAE |
| | QNFPGLESIDLDVKKGEFVVITGLIGSGKTSLLNAMAGFMKRVSGSVDVNGSLLLCDQP |
| | WIQNATVRENILFGLPMDDAKYKNVVYACSLESDLEILPAGDLTEIGERGITLSGGQKAR |
| | INLARAVYADMDIILLDDVLSAVDARVGKHIMAQCIMGILKDKTRILATHQLSLIGSADRI |
| | VFLNGNGSISVGTFEELKSKNEAFSHLMAFNADSHDSEDEKEEEVEDEEEREAVKELVER |
| | QVTRYKSRADEEEIRHDYEANADKDGRLIDDEEKAENGIKFEVYKNYLSIGSGIFKHYSSV |
| | PIVIVLIALSVFCQLFTNTWLSFWSEKKFAHKSNGFYIGFYVMFTFLAAIFLTLEFVLLAYLT |
| | NNASTKLNLMAVQKVLRAPMSYMDVTPMGRILNRFTKDTDTLDNEIGNQIRMLTYFF |
| | SNIVGVLVLCVIYLPWFAIAIPFLGFIFVAIGNFYQASAREIKRLEAVQRSFVYNNFNETLS |
| | GMKTIKAYQAEERFLEKNDTYVNNMNEAYYITIANQRWLSIHLDFVAAGFALIICFLCVF |
| | RVFKISAASVGLLLSYVLQIAGQLSMLVRTYTQVENEMNSVERICEYALYLPEEAPYNISE |
| | TKPADSWPEHGAVRFENVSLAYRPGLPLVLKNLSADIKPKEKIGICGRTGAGKSSIMTAL |
| | YRLSELNGGKIEIDGVDISKLGLNDLRSKLSIIPQDPVLFRGTIRKNLDPFNLSSEDVLWSA |
| | LRRAGLIEESKLEFVKTQKPDAENLHKFHLDREVEDEGSNFSLGERQLISFARAMVRDSKI |
| | LILDEATSSVDYETDSKIQSTIVREFGNCTILCIAHRLRTILNYDRILVLDRGEIKEFDTPWN |
| | LFQSEDGIFKQMCERSNITKDDFKH |
| SEQ ID NO: | ATGGCCGAAGATGAGTCCTCCTCAATTCAAGTTTTTGAGAAAGAAAAGAACGGCAA |
| 1033 | GTCCCATGCCATGATTGAAGAAGCTCAACCAGTTGAGTATATGAAGCAAAGAAGGC |
| | TGTTCTCCTTCCTGTTCTCTAAAAAGGTTCCACCAATTCCAACTCCAGACGAAAGAAA |
| | ACCATATCCATTCAGAAAGGCCAACATCATCTACAAGATTTTCTTTTGGTGGCTGAT |
| | GCCATTGATGAACACTGGTTACAAAAGAACCTTGCAACAAGAGGATTTGTGGTACT |
| | TGGATGGTGATTTGAAGATCGAAGAATATTACGCCATCTTCGAAAAGAGATTGGCT |
| | AAGAGAACTCAAAAGGCTAGAGAAGCTCATTTGAAGTTGCTGGAAGAGAAGAAGA |
| | AAAACGGTACTTTCGATCCAAACGAGGACAATGAATTTGAATTCGAATACCCCAGA |
| | TACTCCTTGGTTTGGGCTTTGTTTGATACTTTCAAGTGGGAGTACTCTCTGTCCATAG |
| | TTTTTGTTGCTTTGGCTGATGTTGGTTTCACTTTGAATCCTTTGTTGTCCAAGGCTTTG |
| | ATCGATTTCGTTGAAGATAGAGTCTTGGGTTACAAGACCAACATTGGTCATGGTGTT |
| | GGTTATGCTATTGGTTGTTCTGCTTTGGTTTCCGTTTCTGGTATTTTGATCAACCACTT |
| | CTTCAACTTGTCTACCCAAGTTGGTGCTAAATCTAAAGCTACTTTGACCAAGGCTAT |
| | GTTGGAGAAGTCTTTTAAGTTGAACGCTAAGGGTAGACATAACTACCCAGCTTCTA |
| | AGATTACTTCCATGTTGGGTACTGATTTGTCCAGAGTTGACTTAGGTATTGGTTTCC |
| | AACCTATTGCTATCGTGTTTCCAATTCCAGTTGCTATTTCCATTGCCTTGTTGATCGTT |
| | AATATCGGCGTTTCTTCTTTGGCCGGTATTGGTATTTTCATTATCTCCACCATTATTAT |
| | CGCTCTGGCTACCAAGAAGTTGTTCTCTTACAGAAAGAAGATCACCAAGTTCACCGA |
| | CTCTAGAATCAATTACATGAAGGAACTGTTGAACAACGTCAGGATCATCAAGTATTA |
| | TTCTTGGGAGCCATCCTACAAAGAAACCATTGCTGATGTTAGAACCTCCGAGATGTA |
| | CAACATTTTCAAGTTGCAGATCCTGAGGAACTTCTTGACTGCTTATGCTGTTTGTTTG |
| | CCACAGATCTCTTCTATGGTTTCCTTCTTGGTTATGTACGCCGTTGATAAGAATAGAT |
| | CCGCTGGTCAAATTTTCGCCTCTTTGTCTTTGTTCAACGTCTTGTCCCAACAGATTAT |
| | GATGTTGCCATTGGCTTTGGCTACTGGTTCTGATGCTTTAGTTGGTATCGATAGAGT |
| | TAGAGGTTTGTTGCAATCTGGTGAAGATGATCCAAAGGACAGAGAATCTTCTTACG |
| | TTGATGTTGACGAACTGATCGAAAAGAAGTTGGCCATCTCTGTTAGAAACGCTACTT |
| | TCCAATGGAAAACCTTCGAACAAATCGACGAATCTGTCTCCCCATCTAAAGAAGAA |
| | GAGGAAAAAGAAAAGCAGATCGAGAGAGAGGAAGAACGTTTGAACAACATTAAC |
| | AAGCAGTTGTCCGGTAACTTCGACCAATCTTCTTCATTGTCTGTTAAGCACACTAAGT |
| | TCCCAGGTTTGAAGCACTTGGATTTCGATATTAAGCAGGGTGAGTTCATTATCATCA |
| | CCGGTATTATTGGTTCCGGCAAGTCATCTTTGTTGAATGCTTTGGCAGGTTTCATGG |
| | ACAAAGAAGAGGGCGAATTGAAAATCAACGGTTCTTTGTTGTTGTGTGGTTACCCA |
| | TGGATTCAAAATGCTCCAGTCAAAGAGAACATCTTGTTCGATTCTGAATACGACGA |
| | GAAGAAGTACAAGGATACAATCTACGCTTGTTCCTTGGATGCCGATTTGGATATTTT |
| | GCCAGCTGGTGATAGAACCGAAATTGGTGAAAGAGGTATTACTTTGTCTGGTGGTC |
| | AAAAGGCAAGAATCAACTTGGCTAGAGCTGTTTACGCTGTTAACGATATCATCTTGT |
| | TGGACGATGTTTTGTCTGCTGTTGATGCTAGAGTTGGTAAGCACATTATGGATAACT |
| | GTTTCATGGGTCTGTTGAAGGATAAGACCAGAATTTTGGCTACCCACCAATTGTCTA |
| | TGATTAACTCTGCTGATAGGGTCATCTTCTTGAATGGTGATGGTACTGTTGATATCG |
| | GTACTCCAGATGAGTTGTTGAAATCTAATGCTGCCTTCCTGAACCTGATGGAATTTT |
| | CTAATGACGAAAAGAACACCGAAGAGGAACAAAAAGAAATGAACGATGAAGAGG |
| | ACAAAGAACTGAAGAGACAAATGACCGAAAAGTCCTTGTTGAACGATAACGACGA |
| | AGATGACGAAGAATCCAGAAAGGATTTCACTTCTAAAACCGGTGAAGCCCAATTGA |
| | TCCAAAAAGAAGAGAGAGCCATTAACGGCATCTCATTCTCTATCTATAAGAACTACG |
| | TTATGGCTGGTTCCGGTGCTTTGAAAGCTGGTATGACTCCAGTATTCTTTTTCTTCGT |
| | TATTCTGGCCACTTTCTTCCAGTTGTTCACTAATACTTGGTTGTCTTTCTGGACCGAA |
| | GAAAAGTTTCCAGGTAGATCTTCTGGTTTCTACATTGGCTTGTACGTTGCTTTCACTT |
| | GCTTGACCATTATCTTCGTTTCTACCGAGTTTTCCCTGATCGTTTTCATTACCAACAA |
| | GGCCTCTAAGCTGTTGAATATTGCTGCTGTTACCAACTTGTTGCATGCTCCAATGTCT |
| | TTCTTTGATACCACTCCAATTGGTAGGATCTTGAACAGATTCACTAAGGATACAGAT |
| | GCCTTGGATAACGAAATCTCTCAACAATTGAGGCTGTTCATCTACCCAACTGCTAAT |
| | GTTTGTGGTGTCTTGATTCTGTGCATTATCTACTTGCCATGGTTCGCTATTGCTGTTC |
| | CATTCTTAGTTGCTTTGTTCATTGGTTTCGCTAACTTCTATCAAGCCTCCTCTAGAGA |
| | AATCAAGAGATTAGAAGCTTTGGCCAGGTCTTTCGTTTACAACAACTTTAACGAAAC |
| | CCTAGGTGGTATGACCACCATTAAGAGTTTTAAGGCTGAATCCAGGTTCCTGATCAA |
| | AAACAACTTGTACATCAACAGGATGAACGAGGCCTACTTTATCTCCTTGTCTAATCA |
| | AAGATGGTTGGGTATCCATTTGGATTTGGTTGCTTCAGCTTTCGCTTTGATTATAGC |
| | CTTGTTGTCCGTTACCAGACAATTCCAAATTTCTGCTGCTTCTGTTGGTTTGTTGGTG |
| | TCATACGTTATGCAAATTGCTGGTCAGTTGTCGTTGTTGATTAGAGCTATGACTCAA |
| | GTCGAAAACGAGATGAACTCTGTTGAAAGATTGGATTACTACGCCTTCCATTTGCCA |
| | TCTGAAGCACCATTTGATATTCCAGAAACTGCTCCACCACCAACATGGCCACAACAT |
| | GGTGTAGTAGAATTCAAGAATGTCTCCTTGGCTTATAGACCAGGTTTGCCATTAGTC |
| | CTGAACAATATCTCATTTTCCGTTAAGGCCGGTGAAAAGATAGGTATTTGCGGTAG |
| | AACTGGTGCTGGTAAATCTTCTATTATGACTGCCTTGTACAGATTGGCTGAATTGGC |
| | TAATGGTGAGATTAACATCGACGGTATTAACATTGCCAAGATCGGTTTGAACTCCTT |
| | GAGATCCAAGTTGTCCATTATTCCACAAGATCCAGTTTTGTTCAGGGGTAACATTAG |
| | AAAGAATCTGGACCCTTTCAACAAGCACAACGATGATGAATTGTGGGGTGCTTTAA |
| | GAAGGTCTGGTCTAATTGAAGAATCCGAGTTGTCTAAGGTTAAGTGTCAAGCTTTG |
| | ACTGATCCACAATTGCACAAGTTTCACTTGGATCAGGTTGTAGAAGATGATGGCTCT |
| | AATTTCTCCTTGGGTGAAAAACAATTGATTGCATTGGCAAGAGCCGTTGTCAGAAA |
| | CTCCAAGATTTTGATTTTGGATGAAGCCACCTCCTCCGTTGATTACGAAACTGATGC |
| | TAAGATTCAAAAGACCATCGTGCAAGAATTCTCTTCCTGTACCATTTTGTGCATTGCC |
| | CATAGATTGAAAACCATCGTTGACTACGATAGAATCTTGGTTTTGGATAAGGGTCA |
| | AGTCCAACAATTCAATACTCCATGGGTCCTGTTTAACAAAGAAGGTATCTTCCAAAA |
| | GATGTGCGAGAGATCTAAAATTACCGCTTTGGACTTCAACCGTAAGAGCTAA |
| SEQ ID NO: | MAEDESSSIQVFEKEKNGKSHAMIEEAQPVEYMKQRRLFSFLFSKKVPPIPTPDERKPYP |
| 1034 | FRKANIIYKIFFWWLMPLMNTGYKRTLQQEDLWYLDGDLKIEEYYAIFEKRLAKRTQKA |
| | REAHLKLLEEKKKNGTFDPNEDNEFEFEYPRYSLVWALFDTFKWEYSLSIVFVALADVGF |
| | TLNPLLSKALIDFVEDRVLGYKTNIGHGVGYAIGCSALVSVSGILINHFFNLSTQVGAKSK |
| | ATLTKAMLEKSFKLNAKGRHNYPASKITSMLGTDLSRVDLGIGFQPIAIVFPIPVAISIALLI |
| | VNIGVSSLAGIGIFIISTIIIALATKKLFSYRKKITKFTDSRINYMKELLNNVRIIKYYSWEPSYK |
| | ETIADVRTSEMYNIFKLQILRNFLTAYAVCLPQISSMVSFLVMYAVDKNRSAGQIFASLSL |
| | FNVLSQQIMMLPLALATGSDALVGIDRVRGLLQSGEDDPKDRESSYVDVDELIEKKLAIS |
| | VRNATFQWKTFEQIDESVSPSKEEEEKEKQIEREEERLNNINKQLSGNFDQSSSLSVKHT |
| | KFPGLKHLDFDIKQGEFIIITGIIGSGKSSLLNALAGFMDKEEGELKINGSLLLCGYPWIQN |
| | APVKENILFDSEYDEKKYKDTIYACSLDADLDILPAGDRTEIGERGITLSGGQKARINLARA |
| | VYAVNDIILLDDVLSAVDARVGKHIMDNCFMGLLKDKTRILATHQLSMINSADRVIFLN |
| | GDGTVDIGTPDELLKSNAAFLNLMEFSNDEKNTEEEQKEMNDEEDKELKRQMTEKSLL |
| | NDNDEDDEESRKDFTSKTGEAQLIQKEERAINGISFSIYKNYVMAGSGALKAGMTPVFF |
| | FFVILATFFQLFTNTWLSFWTEEKFPGRSSGFYIGLYVAFTCLTIIFVSTEFSLIVFITNKASK |
| | LLNIAAVTNLLHAPMSFFDTTPIGRILNRFTKDTDALDNEISQQLRLFIYPTANVCGVLILC |
| | HYLPWFAIAVPFLVALFIGFANFYQASSREIKRLEALARSFVYNNFNETLGGMTTIKSFKA |
| | ESRFLIKNNLYINRMNEAYFISLSNQRWLGIHLDLVASAFALIIALLSVTRQFQISAASVGL |
| | LVSYVMQIAGQLSLLIRAMTQVENEMNSVERLDYYAFHLPSEAPFDIPETAPPPTWPQ |
| | HGVVEFKNVSLAYRPGLPLVLNNISFSVKAGEKIGICGRTGAGKSSIMTALYRLAELANG |
| | EINIDGINIAKIGLNSLRSKLSIIPQDPVLFRGNIRKNLDPFNKHNDDELWGALRRSGLIEE |
| | SELSKVKCQALTDPQLHKFHLDQVVEDDGSNFSLGEKQLIALARAVVRNSKILILDEATSS |
| | VDYETDAKIQKTIVQEFSSCTILCIAHRLKTIVDYDRILVLDKGQVQQFNTPWVLENKEGI |
| | FQKMCERSKITALDFNRKS- |
| SEQ ID NO: | ATGTCCAACTCCGCCTACGATATTGAAGATCATCCTGAAGAGGTTAACAAGTACGAT |
| 1035 | GGTTACAACAACGCTGTTGATTCCGAAGTTCAAAGATTGGCTAGACAAATCACCCA |
| | AAACTCCCAATTGTCTTTTCAAGATGACGGTTTTAAGTTGGCTCCAGGTGAATCTAA |
| | TATCGACGGTTTGTCTAGAGTTTCTACTATTGCTCCTGGTGTTAACCCAATGCAAAAC |
| | GTTGAAGAATTGGACCCAAGATTGGATCCTAACTCTGAAGAATTCCAATCCAGATA |
| | CTGGATCAAGAACTTCAAGGCTTTGATGGATAAGGATCCAGATCACTACAAGAACT |
| | ACTCATTGGGTGTTACCTTCAAGAATTTGAGAGCTTCTGGTGAAGCTTCTGATGCTG |
| | ATTATCAAACCACCATTATTAACGCCCCATTCAAGATTGCTAAGCAATATGCTAAAG |
| | CCGTGTTCTCTACTAGATCTGCTAAACAAGCTAACAGGTTCGACATCTTGAAGTCTTT |
| | GGATGGTATAGTTAGACCAGGTGAAGTTTTGGTTGTTTTGGGTAGACCTGGTTCTG |
| | GTTGTACTACTTACTTGAAATCCATTGCCTCTAACACCCATGGTTTTAAGATAGGTCA |
| | AGAGTCCGAAATGTCTTACGAAGGTTTGACCCAAAAAGAGATCAAAAAGCACTTTA |
| | GAGGTGAGGTTGTTTACAACGCCGAATCCGATATTCATTTCCCACATTTGACTGTTT |
| | GGCAAACTTTGACTACTGCTGCTAAATTCAGAACCCCAGAAAACAGAATTCCAGGT |
| | ATCTCAAGAGAAGATTACGCTAACGCTTTGACCGAAGTTTTTATGGCTACTTATGGT |
| | TTGTCCCATACCAAGAATACCAAGGTTGGTTCAGAATTGGTTAGAGGTGTTTCTGGT |
| | GGTGAAAGAAAGAGAGTTTCAATTGCCGAAGTTTCTTTAGCTGGTGCTAGATTGCA |
| | ATGTTGGGATAATGCTACTAGAGGTTTGGATGCTGCTACTGCTTTGGAATTCATTAG |
| | AGCTTTAAGAACCTCCGCTGATGTTTTGGATACAACTGCTTTGATTGCTATCTACCAA |
| | TGTTCCCAAGAAGCCTACGATTTGTTCGATAAGGTTTCTGTCTTGTACGAGGGTTAC |
| | CAAATTTTCTTTGGTAGAGCTGATAAGGCCAAAGAATACTTCATTAACATGGGTTGG |
| | GAATGCCCACCAAGACAAACTACAGCTGATTTCTTGACTTCTGTTACCTCTCCAAGA |
| | GAAAGAGTTCCAAGAGCTGGTTTTGAAAAGAAGGTTCCAAGAACTCCATCTGAATT |
| | TGCTACTTATTGGAAAGCTTCTCCAGAGTACAAGGCATTGATTGCCGAAATTGATGA |
| | ATCCTTGGCTGCCAATCAAAAGTCCGAATTGAAGGATTTGATCTACGATGCTAAGG |
| | CCTCCAGACAATCTAAAAGAATGAGAAAGACTGACCCCTACACCGTTTCTATTTCAT |
| | TGCAAACTAAGTACCTGTTGGAGAGAGAAGTCTACAGGATTAAGAACAACTTTGGT |
| | TTCCATGGCTTCTCCGCTATTGCTAATTCTTTGATGGCTTTGGTTTTGGCCTCCATCTT |
| | TTACAACATGTCTAAGACTACCGAGTCCTTCTATTCTAGAGGTGCTGCTATGTTTTTC |
| | GCTTGTTTGTTTAACGGTTTCCAGTCCTTCTTGGAGATCTTGTCTTTGTTTGAAGCCA |
| | GACCAATTATCGAAAAGCACAAACAATACGCCTTGTATCATCCAGCTGCTGAAGCTT |
| | TGGCTTCTGTTATTTCTCAATTGCCTTTTAAGGCCTTCTCCTCCTTGATGTTTAACCTG |
| | ATCTATTACTTCATGGTCAACTTCAGAAGAGATCCAGGTAGATTCTTCTTCTACTTGT |
| | TGGCTAACGTTACTTCTACCTTCACCATGTCTCATTTCTTCAGATTGATCGGCTCTAT |
| | GTCATCTACTTTGCCACAAGCTTTGGTTCCAGGTCATATAGTTTTGTTGGGTTTGTCC |
| | ATGTTCGTCGGTTTTACTATTCCAGTCAACTACATGTTAGGTTGGTGCAGATGGATT |
| | AACTACATTAACCCATTGGCTTACGCTTTCGAAGCTTTAATGGCTAACGAATTCCAT |
| | GGTTTGAGATACGCTTGTTCTGCATTTTTGCCAGATAACCCAGATAATCATCCAGAT |
| | TGGCCAGCTAAATCTTGGATCTGTAATGCTGTTGGTGCTGTTGCCGGTGAAGCTACT |
| | GTTTCAGGTGATGCTTACTTAGATGCTGCTTACTCCTACTCTAATTCTCATAAGTGGC |
| | GTAACTGGGCTATTACTTTTGCCTTCTGTATTTTCTTCTTGGCCACCTACATGATTTTC |
| | GCTGAGTATAATGAATCCGCCAAGCAAAAGGGTGAAATCTTGTTGTTTCAAAGGTC |
| | CACCTTGAAGAAGTTGAAGAAAGAACATAAGGCTGCCAAGAACGATATCGAAGGT |
| | GGTAAATTGAGAGACATCACCGAACAAGATCACGACGAAGAATCTGAACAACACG |
| | TTGATGCTATTCAAGCCGGTAAGGATATTTTCCATTGGAGAGATGTTCATTACACCG |
| | TCAAGATTAAGTCCGAGTACAGGGAAATTTTGTCTGGTGTTGATGGTTGGGTTAAG |
| | CCAGGTACTTTGACTGCCTTGATGGGTGCTTCAGGTGCTGGTAAAACTACTTTGTTG |
| | GATGTCTTGGCTAACAGAGTTACTATGGGTATCGTTACTGGTAACATGTTCGTTAAC |
| | GGTAGATTGAGGGATTCCTCATTCCAAAGATCTACTGGTTACGTTCAACAACAGGA |
| | CTTGCATTTGCCAACTGCTACTGTTAGAGAAGCCTTGAGATTTTCTGCTTACTTGAGA |
| | CAACCAGCTGAAGTCTCTAAAGCTGAAAAGGATGACTATGTCGAAGAGGTCATTAA |
| | GATCTTGGACATGCAAAAGTATGCTGATGCTGTTGTTGGAGTTGCTGGTGAGGGTT |
| | TGAATGTTGAACAAAGAAAAAGATTGACCATCGGTGTTGAATTGGCTGCTAAGCCT |
| | AAGTTGTTGTTGTTTTTCGATGAACCTACCTCCGGTTTGGATTCTCAAACTGCTTGGT |
| | CTATTTGCCAGTTGATGAGAAAGTTGGCTAATCACGGTCAAGCTATTTTGTGCACTA |
| | TTCATCAACCATCCGCCATCTTGATGCAAGAATTCGATAGATTGCTGTTCTTAGCCAA |
| | AGGTGGTAGAACTGTTTACTTTGGTGACTTAGGTGAAGGTTGCCAAACCTTGATTG |
| | ATTACTTTGAAAAGTACGGTGCTCCAAAGTGTCCTCCTGAAGCTAATCCAGCTGAAT |
| | GGATGTTGCATGTTATTGGTGCTGCTCCAGGTTCTCATGCTAATCAAGATTATCATC |
| | AAGTCTGGTTGGATTCCGCTGAAAGAAGAGATGTATTGTCTGAATTGGACAGGATG |
| | GAAAAAGAGTTGGTTAACATTCCAGTTGACGATTCCGTTTCTCACTCAGAATTTGCT |
| | GCACCATTTTGGGTTCAATTGACTGTTGTTACTGCCAGAGTGTTCCAACAATTTTGG |
| | AGAACACCATCTTACATTTGGGCCAAGATGTTTTTGTCCGTCGTTTCCTCTTTGTTTA |
| | TCGGCTTTATCTTCTTCAGGTCGAAGAACTCTATCCAAGGCTTGCAAAATCAAATGT |
| | TCGCCTTGTTCATGTTCCTGACCATTTTTAACCCACTGCTGCAACAAATTTTGCCCACT |
| | TTTGTTTCTCAGAGGGACTTGTACGAAACTAGAGAAAGACCAGCTAAGACCTTTTCA |
| | TGGAAGGCTTTCATTATCGCTCAATTCATAGCTGAAGCTCCATGGAATGCTTTTGTT |
| | GGTACAGTTGGTTTCTTCTGCTTTTATTACCCAGCTGGTTTCTACAGAAATGCCGAAC |
| | CATATGATGAAGTCAATGGTAGAGGTGCATATGGTTGGTTCTTTGCTGTTTTGTTCT |
| | TCATCTACATTGGTTCTATGGCCCATATGTTGATTGCCCCTTTACAAATTGCTGATTC |
| | TGCTGGTAATTTGGGCTCTTTGTTGTTCACTATGTGTTTGACTTTCTGCGGTGTTTTG |
| | GTTACTAAGGATGCTTTGCCAGGTTTTTGGGTTTTCATGTATAGAGTCTCTCCATTCA |
| | CCTACTTCATCGAAGGTTATTTGACTAATGCTTTGGCCCATAACAAGATCGTCTGTTC |
| | AGAAGAAGAGTTCAGAGTTTTGTCTCCACCAGATGGTTTGACTTGCCAAGATTATTT |
| | GGGTGACTACATTTCTAAAGCCGGTACAGGTTACTTGCAAGATCCAGAAGCAACTG |
| | GTTCTTGTCAATTTTGTCCAATGTCTAAAACGGACGACTTCTTGGCTCAAGTTCAATT |
| | GGATTATGGTAACAGATGGCGTGATGTCGGTATTTTCATTGCCTTCATTTTCATCAA |
| | CTTGTTCTTCGCCGTCCTGTTTTATTGGTTGGCTAGAGTTCCTAAGAAGTCCGATAG |
| | AGTTAGTACTGAACAACCTGAAGGTGCTGTTAATATGGGTGCTGAATTAGAAAAGA |
| | AAGCCGCCTTGCATAGAACTGCTACAAATGCTGCTTCACAAGCTGCTTCTCAAGGTT |
| | ATGCTCCACAAGTCTATAACGAAAAAGTCGGTTCTGAAGAAGGCTCCTTGGATAAG |
| | GTTGATAACTCTGATTCTTCCAGGTAA |
| SEQ ID NO: | MSNSAYDIEDHPEEVNKYDGYNNAVDSEVQRLARQITQNSQLSFQDDGFKLAPGESNI |
| 1036 | DGLSRVSTIAPGVNPMQNVEELDPRLDPNSEEFQSRYWIKNFKALMDKDPDHYKNYSL |
| | GVTFKNLRASGEASDADYQTTIINAPFKIAKQYAKAVFSTRSAKQANRFDILKSLDGIVRP |
| | GEVLVVLGRPGSGCTTYLKSIASNTHGFKIGQESEMSYEGLTQKEIKKHFRGEVVYNAES |
| | DIHFPHLTVWQTLTTAAKFRTPENRIPGISREDYANALTEVFMATYGLSHTKNTKVGSEL |
| | VRGVSGGERKRVSIAEVSLAGARLQCWDNATRGLDAATALEFIRALRTSADVLDTTALI |
| | AIYQCSQEAYDLFDKVSVLYEGYQIFFGRADKAKEYFINMGWECPPRQTTADFLTSVTS |
| | PRERVPRAGFEKKVPRTPSEFATYWKASPEYKALIAEIDESLAANQKSELKDLIYDAKASR |
| | QSKRMRKTDPYTVSISLQTKYLLEREVYRIKNNFGFHGFSAIANSLMALVLASIFYNMSK |
| | TTESFYSRGAAMFFACLFNGFQSFLEILSLFEARPIIEKHKQYALYHPAAEALASVISQLPF |
| | KAFSSLMFNLIYYFMVNFRRDPGRFFFYLLANVTSTFTMSHFFRLIGSMSSTLPQALVPG |
| | HIVLLGLSMFVGFTIPVNYMLGWCRWINYINPLAYAFEALMANEFHGLRYACSAFLPD |
| | NPDNHPDWPAKSWICNAVGAVAGEATVSGDAYLDAAYSYSNSHKWRNWAITFAFCI |
| | FFLATYMIFAEYNESAKQKGEILLFQRSTLKKLKKEHKAAKNDIEGGKLRDITEQDHDEES |
| | EQHVDAIQAGKDIFHWRDVHYTVKIKSEYREILSGVDGWVKPGTLTALMGASGAGKTT |
| | LLDVLANRVTMGIVTGNMFVNGRLRDSSFQRSTGYVQQQDLHLPTATVREALRFSAYL |
| | RQPAEVSKAEKDDYVEEVIKILDMQKYADAVVGVAGEGLNVEQRKRLTIGVELAAKPKL |
| | LLFFDEPTSGLDSQTAWSICQLMRKLANHGQAILCTIHQPSAILMQEFDRLLFLAKGGRT |
| | VYFGDLGEGCQTLIDYFEKYGAPKCPPEANPAEWMLHVIGAAPGSHANQDYHQVWL |
| | DSAERRDVLSELDRMEKELVNIPVDDSVSHSEFAAPFWVQLTVVTARVFQQFWRTPSY |
| | IWAKMFLSVVSSLFIGFIFFRSKNSIQGLQNQMFALFMFLTIFNPLLQQILPTFVSQRDLY |
| | ETRERPAKTFSWKAFIIAQFIAEAPWNAFVGTVGFFCFYYPAGFYRNAEPYDEVNGRGA |
| | YGWFFAVLFFIYIGSMAHMLIAPLQIADSAGNLGSLLFTMCLTFCGVLVTKDALPGFWV |
| | FMYRVSPFTYFIEGYLTNALAHNKIVCSEEEFRVLSPPDGLTCQDYLGDYISKAGTGYLQD |
| | PEATGSCQFCPMSKTDDFLAQVQLDYGNRWRDVGIFIAFIFINLFFAVLFYWLARVPKK |
| | SDRVSTEQPEGAVNMGAELEKKAALHRTATNAASQAASQGYAPQVYNEKVGSEEGSL |
| | DKVDNSDSSR |
| SEQ ID NO: | ATGTCCGACAACTCCGAAGTCGAATCTTTCAATGCTAAACATGTTGAAGCT |
| 1037 | TCCGGTGGTACTAAGGATCCATATACTGGTGTTCCAGATACCAACTCTGT |
| | TCAATCTTTGGCTAGAACTTTCACCCATATGTCTTTGGCTTCATCCTCTAA |
| | CGATAACGAAATCTATGGTGTTCCTGTTGATGGTCCAGAAGGTGTTGAAT |
| | CTTATAACGCTAAGATCGACCCAAACTCTGAAGAATTTGATGCTCATGCTT |
| | GGATGAGAAACTTGAACAGATTGAGATCTGCTGATCCAGACTACTACAAG |
| | AACATCTCTTTAGGTATGGCTTACAAGAACTTGTCTGCTTTCGGTGATTCT |
| | TCCGATGTTGTTTATCAACCTACCGTCTTGAACGTGTTCCAAAAATCCATT |
| | GAGGACATCTACAGAAAGGTTAGAAAAGCTAGACCATCCGATAAGTTCAC |
| | CATTTTGAAACCTATGGACGGTATTTTGAAGCCAGGTTCTTTGAATGTTGT |
| | TTTGGGTAAACCAGGTTCTGGTTGTTCTACTTTGTTGAAAACCTTGTCCTC |
| | TTCTACCTTCGGTTTCGAAGTTACCAAGGATTCCGTTATTTCCTACGATGG |
| | TATTACCCCAAAAGAGATCGAAAACAACTACAGAGGTGATGTTGTATACC |
| | AAGGTGAAGTTGATATTCACTTCCCACATTTGACTGTGTTCGAAACCTTGA |
| | ACAATGTTGCTTTGTTGACTACTCCAAAGAACAGGATTAAGGGTTTGTCCA |
| | GAGAAGAATTCTCCAAACATATGGCTGAAGCTACTATGGCTATGTATGGT |
| | TTGTCTCATACCAAGAACACTAAGGTCGGTAACGAATTGGTTAGAGGTGT |
| | TTCTGGTGGTGAAAGAAAAAGAGTTTCCATCTGCGAAATCTCATTGGTCA |
| | ATGGTAAGATCGTTTGCTACGATAACTCTACCAGAGGTTTGGATTCTGCTT |
| | CTACATTGTCCTTCATCAAGTCCTTGAAAACCCAATCTAAAGCTTCTGATA |
| | CCACTTCCGTTGTTGCTATCTATCAATGTTCTCAAGATGCCTACGATTTGT |
| | TCGATAACGTCATCGTTTTGGATGAAGGTTACCAGTTGTATAACGGTCCAT |
| | CTAACAAAGCCAAGGACTACTTTATTAAGATGGGTTACGTTTGCCCAGAA |
| | AGACAAACTACTGCTGATTTCTTGACTGCTGTTACTTCTCCAACTGAAAGA |
| | ATCAAGAATACCGAGATGATGGAAAAGGGTATCAAGATTCCAGAAACCTC |
| | CTTGGAAATGTACGAATACTGGAATGCTTCCGAAGAATGCCAAGAATTGA |
| | AGTCCGAAATCGACTACTACTTGTCCCATATCGATTCCTCATTGAAGGATC |
| | AATTCCATCAAGCTCATACTGCTTCTCAAGCTAAAAGAGCTAGACCTTCTT |
| | CTTCTTACTTGTTGACCTTTCAATTGCAGGTCAAGTATTTGTTGCAGAGAA |
| | ACTTCACTAGGATCAAGAACGATATCGGCTTGTCTATGTTTCAAGTCTTGG |
| | GTAATTCTTTCATGGCCTTGATTATTGCCTCCATGTTCTACAAGGTTATGT |
| | ACTACACTAACACCTCCACGTTCTTCTATAGAGGTGGTACTTTGTTTTACG |
| | CCGTCTTGTTTAACTCCTTCTCCTCCTTGTTGGAAATCATGACATTATACG |
| | AAGCCAGGCCAATTATCGAGAAGCAAAAGAATTTGGCAATGTACCATCCA |
| | TCTGCTGAAGCCGTTTCTTCTATTTTGTCTGAAATGCCAGCCAAGATTATT |
| | ACCGCTATTGCCTTCAATATGTTCTACTACTGGATGACCAATTTGAAGAGA |
| | GATGCTGGCGCTTTCTTTTTCTACTTGCTGATGAATTTCGTTTGCCTGTTG |
| | GCTATGTCTCACATCTTTAGATTCATTGGTTCTGCCACTAAGTCTTTTCCA |
| | GGTGCTATGGTTCCAGCTTCTGTTATTTTGTTGGGTATTTCTATGTACGCC |
| | GGTTTCGCTATTCCAAAGACTTCTATGTTAGGTTGGTCTAAGTGGATCTAC |
| | TGGATTAACCCAATTCAATACGGTTTCGAGTCCTTGATGATCAACGAATTT |
| | CATGGTGTCGAATACCCATGCTCATCTTATATTCCATCTGGTACTGGTTAC |
| | TCCGATTTTGATTCAGCTTACAAAACCTGCTCTGTTGTTGGTGCTGTTCCA |
| | GGTTCAGATATTGTCTCTGGTGATTTGTTCCTGAAGTTGTCTTATGGTTAC |
| | GAACATTCTCATAAGTGGCGTGGTTTTGGTGTTGTTTTAGCTTACGCTATT |
| | TTCTTCTTCGGTGTCTACTTGACTTTCACCGAGTATAATGAATCCGCTAAG |
| | CAAAAGGGTGAGATCATTGTCTTTCCACAAGCTGTTGTTCGTAAGATCAA |
| | GAAGATGTCTAAGTCTACCCACGATTTGGAATCAGCTTCAGCATCTGATG |
| | AATCTTACACCGACAAAAAGTTGGTTTCCGATGATTACGATGAATCCCAAG |
| | ATTCCTACAACGATGTTGGTTTGAGTGAATCCGAAGCTACTTTACATTGGA |
| | GAGATTTGTGTTACGACGTCCAAATCAAAGGTGAAACTAGAAGGATCTTG |
| | AACAACGTAGATGGTTGGGTTGCTAAGAACTCTATTACTGCTTTGATGGG |
| | TTCTTCTGGTGCTGGTAAAACTACTTTGATGGATTGCTTGGCTTCTAGAGT |
| | TACCATGGGTGTTATTACCGGTGATATTTTGGTTAACGGTAGATTGAGAG |
| | ATGAGGGTTTCCCAAGATCTATTGGTTACTGTCAACAACAAGACTTGCATT |
| | TGGCTACTGCTACTGTTAGAGAATCCTTGAAATTCTCCGCTTACTTGAGAC |
| | AACCAGCCTCTGTTTCTAAAGAAGAGAAAGACGCTTACGTCGAATCCGTC |
| | ATTAAGATCTTGGATATGCAAAGATACGCTGATGCTGTTGTAGGTGTAATG |
| | GGTGAAGGTTTGAACGTTGAACAAAGAAAACGTTTGACCATCGGTGTAGA |
| | ATTGGCTGCTAAACCTAAGCTGTTGATGTTCTTAGATGAACCTACTTCTGG |
| | TTTGGACTCTCAAACTGCTTGGTCTATTTGTCAGTTGATGAGAAAGTTGGC |
| | TAACCATGGTCAAGCTATTTTGTGCACTATTCATCAACCATCCGCCATGTT |
| | GATTGATCAGTTTGATAGGTTGCTGTTCTTGCAGAAAGGTGGTAAGACTG |
| | CTTATTTTGGTGACTTAGGTGAGGGTTGTAAGACCATGATTGATTACTTTG |
| | AATCTAAGGGTGCTCCAAAGTGTCCACCAAATGCTAATCCAGCTGAATGG |
| | ATGTTGGATATTGTCCAAGCTAGAGATTATCATGAAGCCTGGAAATCTTCA |
| | GACGAGTACAAACAAGTTCATGCTACTTTGGACGAAATGGAAAGAGAATT |
| | GCCAAACATCGTTATCTCCAACGCTGATGATACTTCTGCTTTTGCTGCTTC |
| | ATTTCCAGTCCAACTGTTTTACGTTTACAAGAGAGTCGTCCAACAATATTG |
| | GAGAACTCCAATCTATTTGTGGGCCAAGTTTTTCGTTACTGGTGCTTCTGA |
| | GTTGTTCATCGGTTTTACTTTCTTTAAGGCCAACCACACATTGCAGGGTTT |
| | GAAGAATCAAATGTTGGCCATCTTCATGTTCGTCGTCATTTTTAACCCATT |
| | CTTGCAACAGTACTTGCCAATCTTTACTCAACAGAGAGACTTGTACGAAG |
| | CTAGGGAAAGACCATCTAGAACATTTTCTTGGTACGCTTTCTTGATCGGTC |
| | AAATGTTAGCTGAAATCTTCCCAAACATTTTGTGTGCCATCTTGGGCTTTT |
| | TCTGTTTTTACTACCCAATTGGTTTCGCCTCTAACGCTTCTTATTCTGGTC |
| | AATTGGTTGAGAAGTCTGGCCTGTTTTTCTTCTACTCCTTGATTTTCTTCA |
| | CCTGGATTGGTTCTACAGCATTGATGGTTGCAGCTCCATTTGAAGATCCT |
| | CAAGCTGGTGGTCATTTGGCCAATTTGATGTTTACAATGGCCTTGTCTTTC |
| | AACGGTGTTTTTGTTGGTCCAGGTAAATTGCCAGGTTTTTGGAAGTTTATG |
| | TACCGTGTTTCTCCATTGACCTACTTCGTTGATGGTGCTTTGTCTATAGGT |
| | TTGTCCGATAACAAGGTTGAATGCTCTCAATACGAGTACACTGATGTTCAA |
| | TTGCCAGCTGGTATGACTTGTGGTGAATATTTGTCTCCTTACATCGAGAA |
| | GGTTGGTACAGGTTACATTTTGGATAAGAACGCTACCTCTGAATGCAAGA |
| | TGTGTCAAATTTCTACTACCAACGCCTTCTTGACCACTGTTTCATCTAAAT |
| | ATTCTAGGCGTTGGCGTAACCTGGGTATTTTCATTGCTTACATTGCTTTCA |
| | ACTACGTGATGGCCATGTTCTTATATTGGTGGGCTAGAGTTCCAAAGAAG |
| | GCTAACAGAGTTTCTGACGAAAAAGACTCTTCCAAGGATAAGAAGGACGA |
| | GAAGAAGTAG |
| SEQ ID NO: | MSDNSEVESFNAKHVEASGGTKDPYTGVPDTNSVQSLARTFTHMSLASSSNDNEIYGV |
| 1038 | PVDGPEGVESYNAKIDPNSEEFDAHAWMRNLNRLRSADPDYYKNISLGMAYKNLSAF |
| | GDSSDVVYQPTVLNVFQKSIEDIYRKVRKARPSDKFTILKPMDGILKPGSLNVVLGKPGS |
| | GCSTLLKTLSSSTFGFEVTKDSVISYDGITPKEIENNYRGDVVYQGEVDIHFPHLTVFETLN |
| | NVALLTTPKNRIKGLSREEFSKHMAEATMAMYGLSHTKNTKVGNELVRGVSGGERKRV |
| | SICEISLVNGKIVCYDNSTRGLDSASTLSFIKSLKTQSKASDTTSVVAIYQCSQDAYDLFDN |
| | VIVLDEGYQLYNGPSNKAKDYFIKMGYVCPERQTTADFLTAVTSPTERIKNTEMMEKGI |
| | KIPETSLEMYEYWNASEECQELKSEIDYYLSHIDSSLKDQFHQAHTASQAKRARPSSSYLL |
| | TFQLQVKYLLQRNFTRIKNDIGLSMFQVLGNSFMALIIASMFYKVMYYTNTSTFFYRGG |
| | TLFYAVLFNSFSSLLEIMTLYEARPIIEKQKNLAMYHPSAEAVSSILSEMPAKIITAIAFNMF |
| | YYWMTNLKRDAGAFFFYLLMNFVCLLAMSHIFRFIGSATKSFPGAMVPASVILLGISMY |
| | AGFAIPKTSMLGWSKWIYWINPIQYGFESLMINEFHGVEYPCSSYIPSGTGYSDFDSAYK |
| | TCSVVGAVPGSDIVSGDLFLKLSYGYEHSHKWRGFGVVLAYAIFFFGVYLTFTEYNESAK |
| | QKGEIIVFPQAVVRKIKKMSKSTHDLESASASDESYTDKKLVSDDYDESQDSYNDVGLSE |
| | SEATLHWRDLCYDVQIKGETRRILNNVDGWVAKNSITALMGSSGAGKTTLMDCLASR |
| | VTMGVITGDILVNGRLRDEGFPRSIGYCQQQDLHLATATVRESLKFSAYLRQPASVSKEE |
| | KDAYVESVIKILDMQRYADAVVGVMGEGLNVEQRKRLTIGVELAAKPKLLMFLDEPTS |
| | GLDSQTAWSICQLMRKLANHGQAILCTIHQPSAMLIDQFDRLLFLQKGGKTAYFGDLG |
| | EGCKTMIDYFESKGAPKCPPNANPAEWMLDIVQARDYHEAWKSSDEYKQVHATLDE |
| | MERELPNIVISNADDTSAFAASFPVQLFYVYKRVVQQYWRTPIYLWAKFFVTGASELFIG |
| | FTFFKANHTLQGLKNQMLAIFMFVVIFNPFLQQYLPIFTQQRDLYEARERPSRTFSWYA |
| | FLIGQMLAEIFPNILCAILGFFCFYYPIGFASNASYSGQLVEKSGLFFFYSLIFFTWIGSTAL |
| | MVAAPFEDPQAGGHLANLMFTMALSFNGVFVGPGKLPGFWKFMYRVSPLTYFVDGA |
| | LSIGLSDNKVECSQYEYTDVQLPAGMTCGEYLSPYIEKVGTGYILDKNATSECKMCQIST |
| | TNAFLTTVSSKYSRRWRNLGIFIAYIAFNYVMAMFLYWWARVPKKANRVSDEKDSSKD |
| | KKDEKK- |
| SEQ ID NO: | ATGACCTCTCCATCTGCCTTGGAATCCAATCAATTGTCTACTGAAAGACAGAAGAGG |
| 1039 | CTGTTGTCTTTCTTGATGTCTAAAAAGGTTCCACCAGTGCCAACTCAAGAAGAAAGA |
| | AAACCATATCCAGGTTACAGAGCCAACATTATCAAGCAGTTGTTTTTCTGGTGGTTG |
| | TCCCCAGTTATGAAGGTTGGTTATCAAAGAACATTGCAACCAGACGACATGTTCTAC |
| | TTGACCGATGATATTAAGGTTCAGAAAATGGCCGAAGATTTCTACAGGTACATGTC |
| | CCATGATATTGATAGAGCTAGACAAAAGCACATTGCTGAAAAGTGTGCTAAAAGAG |
| | GTGAAGCTCCAGAAACTACTTCTGTTTCTCCTGAAGAAGATATGAAGGACTTCGAG |
| | ATGTCTAAGTTCTTGACTGTTTGGGCTTTAGCTAAGACTTTTAAGTGGCAGTATACTT |
| | GGGCTTGTGTTTGTTTGGCTTTGTCTAATGCTGGTCAAACTACTATGCCTCTGCTGTC |
| | CAAAAAGTTGATCCAATACGTTGAATTGAAGGCCTATGGTAGAGAACCAGGTGTTG |
| | GTAAAGGTTTGGGTTATTCTTTTGGTACTACTGCCATGGTTTTCATCGTTGGTGTTTT |
| | GATCAACCACTTCTTCTACAGATCTATGATTACTGGTGCTCAAGCTAAAGCTGTTTTG |
| | ACTAAGGCTTTGTTGGACAAGTCTTTCAAGTTGTCTGCTGAAGCTAAACACAAGTAC |
| | TCCGTTGGTAAGATTACTTCCATGTTGGGTACTGACTTGTCTAGAATTGATTTCGCTT |
| | TGGGTTTCCAGCCATTCTTGATCGTTTTTCCAATTCCAATTGCTATCGCCATCGTTATC |
| | TTGGTCATCAATATTGGTGTTGCCTCTTTGGTTGGTGTCGGTATTTTGTTGGTTTTTA |
| | TGATTGGTATCGCCTTCTCTACCGGTAAGTTGTTTGCTTATAGAAAGAAGGCTAACA |
| | AGTACACCGATTCCAGAGTTAACTACATGAAGGAAGCTCTGAACAACCTGAAGATC |
| | ATCAAGTTTTATTCTTGGGAACCACCATACCACAAGAACATTTCCGATATCAGAAAG |
| | AAAGAGATGAGGATCATCTACAGAATGCAGGTCTTGAGAAATATCGTTACCTCTTTC |
| | GCTATGTCCTTGACTTTGTTCGCTTCTATGACTGCATTTTTGGTCTTGTACGCTATCTC |
| | TAACGGTAGAAGAGATCCAGCTTCTATCTTCTCTTCCTTGTCCTTGTATAACGTCTTG |
| | ACCCAACAAGTTTTCTTGTTGCCAATGGCTTTGGCTACAGGTGCTGATGCTTTTATG |
| | GGTATTTCTAGAGTTGGTGATTTCATGGCCCAATCTGAAATCAATCCAGAAGAAAAC |
| | GCTATTGAAGCTCCTCCAGATGTACAAGAGTGGATGGACAAAGAATCTTTGGCTAT |
| | TGATATCGAGAAGGCCTCTTTTGAATGGGAAGTTTTTGAAGATGAGGACGAGAAA |
| | GAAGAGAAGTCCTCTAAAAAGTCTAAGAAGGGAAACAAGAGGAACTCCGAAGAAT |
| | CTGCTATTTTGGATGTTCAAGACTCCGATGTCTCTAGCTCAAAAGGTTTAGATAGAT |
| | CCTCTATTACCGACGGTTCTTCCGAAGAAGATGCACATTTTGAAGGTTTGAAGAACA |
| | TCGACTTCAAGATCAAGAAGGGTGAATTCGTTGTTATCACTGGTATGATCGGTTCCG |
| | GTAAATCTTCTTTGTTATCTGCTATGTCCGGTTTCATGAAGAGAAACTCTGGTTCTGT |
| | TTACGTCAACGGTTCTTTGTTGTTGTGTGGTTATCCATGGGTTCAAAACTCCACTGTT |
| | AAGGACAACATCATCTTCGGTTCTGAATACGACGAAGAAAAGTACAAGAGAGTTAT |
| | CTACGCTTGCTCTTTGGAAGCCGATTTGGAATTATTGCCAGCTGGTGATAGAACCGA |
| | AATTGGTGAAAGAGGAATTACTTTGTCTGGTGGTCAAAAGGCTAGAATCAATTTGG |
| | CTAGAGCTGTTTACGCTAACAAGGACATTATCTTGTTGGATGATGTTTTGTCCGCTG |
| | TTGATGCAAGAGTTGGTAAACATATTATGAACAACTGCCTGCTGGACCTGTTGAAA |
| | GAAAAGACTAGAGTTTTGGCTACCCACCAGTTGTCTTTGATTGGTTCTGCTGATAGA |
| | GTCATCTTCTTGAATGGTGATGGTTCCATTGATGTCGGTAAGTTCGAAGAATTGAAA |
| | GAGAGAAATCCAGGTTTCGGTAAATTGATGGCTTTCAACTCCGAATCCAAAGAAGA |
| | AAAAGAAGGCAACGAAGAAGAATTGGAACAATCCGCTGAACAAGAATTGATCGAA |
| | GAGGATAAGAGAAACATCGAAAGACAGTTGACTAGAAGATCCACTAGAACTGTTA |
| | CTACTGTTGGTGCTTTTCCAGAAGATGTCGACGAAGAGGAAGAAGAGGACGAAGA |
| | AGGTAGACATAGGGAGTATAATTTGGATGAAGAAGCTGACGGCAAGTTGATTACT |
| | GATGAAGAAAGAGCAGTTAACGCCATCTCTAAGAAAATCTACGCTAGATACTTCCA |
| | GCTAGGTTCTGGTAAATTCACTCCATGGGCTATGTTGCCTTTGTTATTGACTTTTATT |
| | GTCCTGGCTACCTTCTCTCAAATTTTCACTAATACCTGGTTGTCCTTCTGGACTGAGT |
| | ACAAATTTGATAAGCCTAACAAGTTCTACATCGGCATCTACATCATGTTCACGTTCTT |
| | GTCCTTCATTTTGTTGACCTGCGAATTCATCATCTTGGTCTATATTACCAATACCGCC |
| | TCCGTTCAAATGAACGTTTTAGCAGTTCAAAAGGTCTTGCATGCTCCAATGTCTTTTA |
| | TGGATACAACTCCAATGGGCAGAATCTTGAACAGATTCACTAAGGATACCGATGTC |
| | TTGGATAACGAAATCGGTGATCAATTGAGGTTCTTCTTGTTTGTTTTCGCCAACATCA |
| | TTGGTGTTATCGTCTTGTGCATTATCTACTTGCCTTGGTTTGCTATTGCTGTTCCATTT |
| | TTGGGTATGCTGTTCGTTTCAATTGCCGATTATTACCAAGCTTCTGCCAGAGAAGTT |
| | AAGAGATTGGAAGCTGTTCAAAGGTCCTTGGTTTACAACAACTTCAACGAAACATT |
| | GTCTGGTATGGCTACCATTAAGGCTTACAAAGCTACCGAAAGATTCATCGACAAGA |
| | ACAACTACCTGATCAACAGAATGAACGAAGCCTACTACATTACCATTGCTAATCAAA |
| | GATGGTTGGCCATCCACATGGATTTTGTTGCTACTTTATTCGCCTTGCTGATTGCTTT |
| | GTTGTGCGTTTTTAGAGTGTTCAACGTTTCCGCTTCTACTGTCGGTTTGATTTTGTCT |
| | TACGTCTTGCAAATAGCCGGTCAGTTGTCTATGTTGATTAGAACTTTCACCCAGGTC |
| | GAAAACGAAATGAATTCTGCTGAAAGGTTGAACTCTTACGCTACTTCTTTGCCAACA |
| | GAAGCCCCTTACATTATTACTGAAAATGCTCCACCACCAAACTGGCCATCTCAAGGT |
| | ACTATAGTTTTTGATTACGCTTCCTTGGCTTACAGACCAGGTTTGCCATTGGTTTTGA |
| | AGAATTTGAACTTCTCCGTTAAGCCCATCGAAAAGATTGGTATTTGTGGTAGAACTG |
| | GTGCTGGTAAGTCATCTATTATGACTGCCTTGTACAGGTTGTCCGAATTGGATTCAG |
| | GTAAGATTGAAATCGATGGCATCGACATCTCTAAATTGGGCTTGAAGGATTTGAGG |
| | TCCAAGTTGTCAATTATCCCACAAGATCCAGTCTTGTTTAGAGGCACTATCAGATCT |
| | AACTTGGATCCATTCAATGAACACGACGACGAAAGATTGTGGGATGCTTTGAGAAG |
| | AACTGGTTTGATTGAAGGTTCCAGATTAGAAGCCGTTAAGAGACAAGCTAAGTCCA |
| | AGTCATCAAGCAACAACATGTCTGAAAAATCCGCCGAAAAGTCTACTACTACTCCAG |
| | ATTCTTTAGCCTTGCATAAGTTCCATTTGGATCAAACTGTTGAGGATGACGGTTCTA |
| | ATTTCAGCTTAGGTGAAAGGCAATTGATCGCTTTTGCTAGAGCTTTGGTTAGAGACT |
| | CCAAGATTTTGATCTTAGATGAAGCCACCTCCTCCGTTGATTACGAAACTGATTCTA |
| | AGATCCAAGAAACCATCATCAGGGAATTCAAGGATTGCACCATTTTGTGCATTGCCC |
| | ATAGGTTGAAAACTATCATCAACTACGATAGGATCCTGGTTTTGGATAAGGGTGAA |
| | GTTAGAGAATTTGATACCCCATGGAACTTGTTCAACTCTAAGGGTTCTATTTTCAAG |
| | CAGATGTGCGAAAGATCTAACGTTACCGAACAAGATTTCGGTCAGTGA |
| SEQ ID NO: | MTSPSALESNQLSTERQKRLLSFLMSKKVPPVPTQEERKPYPGYRANIIKQLFFWWLSP |
| 1040 | VMKVGYQRTLQPDDMFYLTDDIKVQKMAEDFYRYMSHDIDRARQKHIAEKCAKRGE |
| | APETTSVSPEEDMKDFEMSKFLTVWALAKTFKWQYTWACVCLALSNAGQTTMPLLSK |
| | KLIQYVELKAYGREPGVGKGLGYSFGTTAMVFIVGVLINHFFYRSMITGAQAKAVLTKAL |
| | LDKSFKLSAEAKHKYSVGKITSMLGTDLSRIDFALGFQPFLIVFPIPIAIAIVILVINIGVASLV |
| | GVGILLVFMIGIAFSTGKLFAYRKKANKYTDSRVNYMKEALNNLKIIKFYSWEPPYHKNIS |
| | DIRKKEMRIIYRMQVLRNIVTSFAMSLTLFASMTAFLVLYAISNGRRDPASIFSSLSLYNVL |
| | TQQVFLLPMALATGADAFMGISRVGDFMAQSEINPEENAIEAPPDVQEWMDKESLAI |
| | DIEKASFEWEVFEDEDEKEEKSSKKSKKGNKRNSEESAILDVQDSDVSSSKGLDRSSITDG |
| | SSEEDAHFEGLKNIDFKIKKGEFVVITGMIGSGKSSLLSAMSGFMKRNSGSVYVNGSLLL |
| | CGYPWVQNSTVKDNIIFGSEYDEEKYKRVIYACSLEADLELLPAGDRTEIGERGITLSGGQ |
| | KARINLARAVYANKDIILLDDVLSAVDARVGKHIMNNCLLDLLKEKTRVLATHQLSLIGSA |
| | DRVIFLNGDGSIDVGKFEELKERNPGFGKLMAFNSESKEEKEGNEEELEQSAEQELIEED |
| | KRNIERQLTRRSTRTVTTVGAFPEDVDEEEEEDEEGRHREYNLDEEADGKLITDEERAVN |
| | AISKKIYARYFQLGSGKFTPWAMLPLLLTFIVLATFSQIFTNTWLSFWTEYKFDKPNKFYI |
| | GIYIMFTFLSFILLTCEFIILVYITNTASVQMNVLAVQKVLHAPMSFMDTTPMGRILNRFT |
| | KDTDVLDNEIGDQLRFFLFVFANIIGVIVLCIIYLPWFAIAVPFLGMLFVSIADYYQASARE |
| | VKRLEAVQRSLVYNNFNETLSGMATIKAYKATERFIDKNNYLINRMNEAYYITIANQRW |
| | LAIHMDFVATLFALLIALLCVFRVFNVSASTVGLILSYVLQIAGQLSMLIRTFTQVENEMN |
| | SAERLNSYATSLPTEAPYIITENAPPPNWPSQGTIVFDYASLAYRPGLPLVLKNLNFSVKPI |
| | EKIGICGRTGAGKSSIMTALYRLSELDSGKIEIDGIDISKLGLKDLRSKLSIIPQDPVLFRGTI |
| | RSNLDPFNEHDDERLWDALRRTGLIEGSRLEAVKRQAKSKSSSNNMSEKSAEKSTTTPD |
| | SLALHKFHLDQTVEDDGSNFSLGERQLIAFARALVRDSKILILDEATSSVDYETDSKIQETII |
| | REFKDCTILCIAHRLKTIINYDRILVLDKGEVREFDTPWNLFNSKGSIFKQMCERSNVTEQ |
| | DFGQ* |

### EXAMPLES

### Materials and Methods

Chemicals used in the examples herein, e.g. for buffers and substrates, are commercial products of at least reagent grade. Water utilized in the examples was de-ionized, MilliQ water.

Promoters and plasmids used throughout the examples were, unless otherwise characterized, standard promoters and plasmids abundantly known to the skilled person.

### Example 1: Analytical Methods for Thebaine, Oripavine, Nororipavine, and Glucosylated Nororipavine and Oripavine

### HPLC Analysis of Thebaine/Oripavine Samples

Stock solutions of oripavine and nororipavine were prepared in 0.1% (v/v) formic acid in H₂O at a concentration of 5 mM. Standard solutions were prepared at concentrations of 50 μM, 100 μM, 250 μM and 500 μM from the stock solutions. Samples were injected into an Agilent 1290 Infinity I UHPLC with a binary pump (Agilent Technologies, Palo Alto, CA, USA). Separation was achieved on a Kinetex F5 column (100×2.1 mm, 1.7 μm, 100 Å, Phenomenex, Torrance, CA, USA) using 0.05% (v/v) formic acid in H₂O and 0.05% (v/v) formic acid in acetonitrile as mobile phases A and B, respectively using the time-gradient as shown in Table 3.

TABLE 3

| Time (min) | % B |
|---|---|
| 0.0-1.0 | 2% |
| 1.0-4.8 | 2-30% |
| 4.8-5.0 | 30-100% |
| 5.0-6.0 | 100% |
| 6.0-6.2 | 100-2% |
| 6.2-6.5 | 2% |
| 1.5 min postrun | 2% |

Stock solutions of O-glycosyl nororipavine, O-glycosyl oripavine, nororipavine and oripavine eluted at 2.13, 2.40, 2.8 and 3 min, respectively.

The injection volume was 1 μL and the flow rate was 600 μL/min. The column temperature was maintained at 30° C. The liquid chromatography system was coupled to an Agilent 1290 diode array detector (Agilent Technologies, Palo Alto, CA, USA). UV-spectra were acquired at 220, 254 and 285 nm. 285 nm used for the quantification of nororipavine, oripavine and O-glycosyl-compounds.

### Example 2—Microtiter-Based Screening of Strains Overexpressing Eflux Transporters

sOD655 is a Saccharomyces cerevisiae yeast strain comprising recombinant polynucleotide sequences expressing P450 N-demethylases (SEQ ID No. 772 and SEQ ID No. 876), CPR (SEQ ID No. 292), PUP uptake transporters (SEQ ID Nos. 613, 473 and 537), ScUGP1 (SEQ ID No. 900), and a UDP-glucose glycosyltransferase (UGT) (SEQ ID No. 878) which enables it to convert oripavine to nororipavine and nororipavine glucoside. The background of S. cerevisiae sOD655 is similar to the commonly available strain S288C (genotype MATa his3Δ0leu2Δ0 ura3Δ0) (see the Saccharomyces Genome Database (SGD)).

Different putative efflux transporters were overexpressed in sOD655 using plasmid RPB15, an empty control plasmid that is the negative control for the data in this example. RPB15 is a derivative of vector p416TEF (Mumberg, 1995). YOR1 (SEQ ID No. 872) is used throughout the microtiter-based screening as a positive control, as it was discovered that it has an effect on glucosylated nororipavine excretion. Thus, a large number of the transporters are also ABC transporters with homology to YOR1 (see example 5). It is expected that upregulation of the native YOR1 via overexpression of transcription factors PDR1, PDR3, YRR1, and PDR8 (SEQ ID Nos. 902, 904, 906 and 908 respectively) would also improve excretion of nororipavine-gly, since these have been shown experimentally to increase YOR1 transcription in yeast (see https://www.yeastgenome.org/locus/S000002981, https://www.yeastgenome.org/locus/S000003513, and https://www.yeastgenome.org/locus/S000000101, Katzmann et al 1995). All experiments were run in at least triplicates. The strains were grown aerobically in deep well culture at 30° C. for 24 h in Delft media pH 5.5 or 4.5, followed by addition of 3 mM oripavine. After 72 hours supernatant and total broth samples were prepared. For the total broth sample an aliquot of the total cell culture was taken and analyzed. For the supernatant sample, first the cells were removed from the cell culture by centrifugation, and then an aliquot of the supernatant was taken and analyzed. Opioids were quantified as described in Example 1. The opioid outside concentration reported is the opioid measurement from the supernatant sample. The concentrations (per working volume) of a certain opioid contributed by the opioid retained inside the cell were calculated as [Opioid in total broth]−[Opioid in supernatant].

Overexpression of certain efflux transporters results in a higher ratio of extracellular to intracellular amounts glucosylated nororipavine as compared to the control. This indicates that these transporters are involved in transporting glucosylated noripavine out of the cells, which in some cases improves the amount glucosylated nororipavine produced as well.

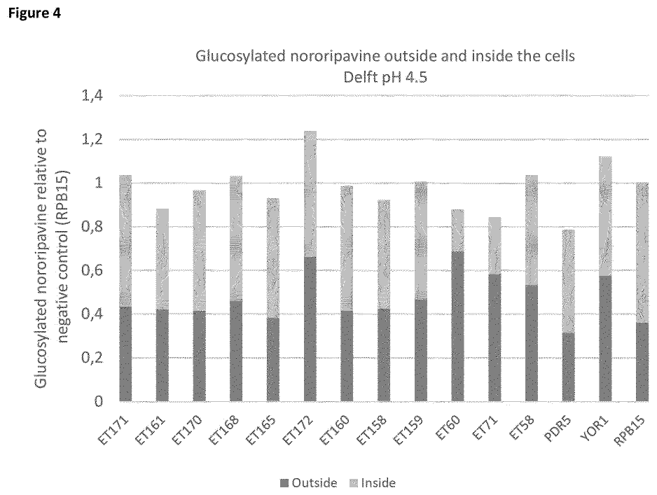

FIG. 4 shows the ratio of extracellular to intracellular concentrations (outside the cell versus inside the cell) of glucosylated nororipavine, and the sum of total product relative to the PRB15 negative control (with no exogenous transporter). Cultures were grown at pH 4.5.

One can see that several ABC transporters showed positive results for improved excretion at pH 4.5, in particular ET60, ET71, ET58, and YOR1 (SEQ ID Nos. 910, 912, 914 and 872 respectively). Similarly, when ratios of nororipavine-glucoside outside versus inside of the cell were compared, all of the transporters showed a higher percent excretion as compared to the no-transporter control, with PDR5 being only slightly above the negative control (40% extracellular nororipavine-gly as compared to 36% for the control).

A similar experiment was conducted at pH 5.5. The following table 4 shows the percent of extracellular glucosylated nororipavine for the best-performing efflux transporters.

TABLE 4

| Transporter gene | Percent excretion |
|---|---|
| No exogenous transporter (RPB15) | 41.5-43.0% |
| ET60 | 80.8-80.9% |
| ET161 | 53.5% |
| ET58 | 56.7-58.1% |
| YOR1 | 54.1-55.9% |
| ET63 | 56.3% |
| ET71 | 69.4-71.4% |
| ET170 | 52.7% |
| ET97 | 51.0% |
| ET82 | 52.1% |
| ET81 | 52.1% |

Thirty-two other screened efflux transporters displayed higher than the negative control but at or lower than 50% excretion, whereas three transporters appeared to be lower than the negative control. At pH 5.5, the best performing efflux transporter was ET60.

Eight additional transporters were tested at pH 4.5 and 5.5 and compared to YOR1 and the negative control (RBP15). At pH 5.5 in this experiment, the negative control had 40.1% glucosylated nororipavine outside of the cell whereas the best efflux transporters appeared to be ET83 (59.7%) and ET72 (58.5%) (SEQ ID Nos. 930 and 932 respectively) where the YOR1 positive control excreted 48.7 percent, and six of the new transporters did not appear to perform better than the control. At pH 4.5 the efflux percentages of the same transporters were 58.0% for ET72, 46.4% for YOR1, 56.7% for ET83, and 36.0% for the negative control; and all 8 of the new transporter proteins tested appeared to be higher than the negative control.

Fourteen additional transporters were tested for nororipavine-gly (nororipavine glycoside) efflux in microtiter plates as described above. At pH 5.5, all fourteen showed higher activity than the control (vector only) with the best performer being ET47 (61.75% excretion) (SEQ ID No. 934). At pH 4.5, thirteen out of the fourteen showed higher activity than the negative control, with ET120 (SEQ ID No. 936) having the highest excretion at 57.24%.

Twenty-seven additional transporters were tested in a similar manner as above, in microtiter plates. At pH 5.5, RPB15 showed 38.8% excretion whereas five of the transporters were similar to or below the negative control efflux percentage. The best transporters under these conditions were ET212 (70.0%), ET193 (74.7%), and ET208 (62.4%) (SEQ ID Nos. 938, 942 and 940 respectively).

At pH 4.5, the no-transporter control excreted 35.5% whereas ET212 was 67.2%, ET208 excreted 57.4%, and ET193 excreted 72.6% of the glucosylated nororipavine. ET60 was only slightly higher in excretion than ET193 in the conditions tested, in regards to percent excretion. ET212 also produced a high total bioconversion of oripavine to nororipavine and glucosylated nororipavine as compared to other transporters, again comparable to ET60. It was noted that the transporters with the lowest activity showed very low homology to YOR1 (25% or lower), whereas high activity transporters ET212 and ET193 show 42.5 and 43.5% identity to YOR1, and 47.9 and 53.8% identity to ET60. Overall, the majority of the ABC transporters tested, especially those with homology to YOR1, worked under the conditions tested for efflux of glycosylated-nororipavine.

Overall, under the conditions tested, ET60 resulted in the highest nor-gly (nororipavine glycoside) efflux, but many other ABC transporters showed activity as well, in particular those that show homology to YOR1. Total bioconversion to nororipavine and glucosylated nororipavine increased by 10.5% percent at pH 4.5 and 22.6-26.4% at pH 5.5 as compared to the no transporter control, when using ET60. ET212 increased total bioconversion at pH 5.5 by approximately 25% as well.

One skilled in the art would recognize that the efflux transporters described herein would work in other cellular fermentations (from sugar) or bioconversion systems, e.g. from thebaine, as described in WO 2021/069714 A1, WO2020078837, and WO2018229306. Enzymes and fermentation conditions as well as required substrates are described in the referenced articles. As an example, if one were converting thebaine to nororipavine-glycoside, then thebaine specific uptake transporters would be used rather than oripavine uptake transporters; and O-demethylases and suitable CPR partners for the O-demethylase would be required. Suitable thebaine uptake transporters include, but are not limited to: PupL (T105), T161, or those improving thebaine to northebaine bioconversion in Tables 6 and 8 of WO2020078837 such as SEQ ID Nos. 307, 317, 317, 311, 733, 735 and 461. Suitable O-demethylase enzymes (and accompanying CPRs) for conversion of thebaine to oripavine or northebaine to nororipavine include but are not limited to: SEQ ID Nos. 222, 224 and 236 when individually expressed in a yeast strain that contains demethylase-CPR Ce_CPR as described in WO 2021/069714 A1, and SEQ ID Nos. 198 and 874 or variants thereof, or additional enzymes that have both N- and O-demethylase activity such as those described in paragraphs 0124-0127 of WO 2021/069714 A1.

### Example 3 Construction of a Stable Yeast Strain for Glucosylated Nororipavine Production

Strain sOD569 was produced by genomic integration using the Saccharomyces cerevisiae gene integration and expression system developed by Mikkelsen, M D et al. (2012). The following genes were integrated into stable loci using amino acid auxotrophic markers and hygMX markers: T193 AanPUP3, T109 GfPUP3, T149 AcPUP3, HaCPR E0A3A7, A0A2A4JAM9, UGP1, and KAF3968553. Multiple copies of genes A0A2A4JAM9, T193 AanPUP3, and KAF3968553 were introduced into the genome by Ty integration. The method of Ty genomic integration was modified based on system developed by Maury, J et al. 2016. Common promoters such as Saccharomyces cerevisiae TDH3, TEF1, pALD4, pFBA1, pPGK1, and TEF2 were used to drive expression of these genes. Strain sOD668 is a similar strain except that it expresses an extra (heterologous) copy of the native yeast ABC transporter YOR1 compared to sOD569. Strain sOD918 is similar to sOD569 but contains a copy of the heterologous efflux transporter ET60.

To test if inclusion of an efflux transporter improved conversion of oripavine into nororipavine compounds, or excretion of nororipavine and nororipavine glycoside in bioconversion reactions, fermentations were done in 2 L bioreactor vessels as described in Example 4 using the strains above.

One skilled in the art will recognize that other suitable polypeptides for oripavine conversion to glucosylated nororipavine may be used in combination with the efflux transporters such as those described in WO2021069714 and WO2020078837A1.

Preferred N-demethylases include but are not limited to SEQ ID Nos.: 140, 152, 843, 198, 250, 252, 771, 875 or insect demethylases disclosed in WO2021069714 as SEQ ID NO: 140, 142, 144, 146, 148, 150, 152, 154, 156, 158, 160, 162, 164, 166, 168, 170, 172, 174, 176, 178, 180, 182, 184, 186, 188, 190, 192, 194, 196, 827, 829, 831, 833, 835, 837, 839, 841, 843, 845, 847, 849, 851, 853, 855, 857, 859, 861, 863, 865, 867 and 869.

Preferred CPRs include but are not limited to SEQ ID Nos.: 292, 305 or sequences described in WO2021069714 as SEQ ID NO: 292, 294, 296, 298, 300 or 302.

Preferred PUP transporters for oripavine uptake include but are not limited to SEQ ID Nos. 473, 479, 481, 487, 493, 495, 503, 507, 509, 513, 517, 523, 525, 527, 529, 537, 539, 541, 543, 547, 551, 559, 561, 567, 571, 573, 575, 579, 589, 591, 595, 597, 599, 611, 613, and 617.

Preferred UGTs (data not shown) include but are not limited to SEQ ID Nos. 879, 881, 883, 885, 887, 889, 891, 893, 895, 897.

### Example 4: Fermentation Process Using Stable Production Strain

One skilled in the art will appreciate that many different fermentation conditions, media, and carbon sources would be acceptable for nororpavine-glucose production using strains expressing ABC efflux transporters described in this patent. Non-limiting examples of other bioconversions utilizing oripavine include WO2021069714 and WO2020078837A1.

In this particular example a 2-stage seed culture process was used from frozen glycerol stocks using minimal medium containing 20 g/L of glucose. Cells were grown at 30° C. on an orbital shaker (180 rpm) for c.a. 24 hours at each stage in order to reach a final OD₆₀₀ suitable for inoculation. Approximately 12% inoculum is used, one skilled in the art will know that typical inoculation volumes can range from approximately 3-20%.

### Process Parameters for Batch and Fed-Batch Phases of Cultivation

The fermentation process is a fed-batch process operated with multiple feed rates. During all phases, temperature was kept at 28° C. with stirring at 1100 rpm. pH was controlled at 5.5 using ammonium hydroxide 17% (w/w). Aeration was continuously increased to keep 1.5 vvm.

The first (batch) phase is typically less than 12 hours uses minimal media with 10 g/L glucose, and is typically a fill volume that is one-third to one-half of the total final working volume. Oripavine can be added during the feed phase or optionally during the batch phase. Fed-batch phases include both a glucose-limited exponential growth phase targeting a growth rate of approximately 0.1 h⁻¹ followed by constant feed rates supplying the carbon source. Feed solutions used are made in minimal medium, typically containing 620 g/L glucose. Fermentation length ranges from ca 80 hours to 140 hours. In this particular example the following media compositions were used:
Batch Media: glucose 10 g/L, (NH₄)₂SO₄ 5 g/L, KH₂PO₄ 3 g/L, MgSO₄·7 H₂O 0.5 g/L, trace elements solution 10 mL/L, vitamin solution 12 mL/L.
Fed batch Media: glucose 620 g/L, (NH₄)₂SO₄ 5 g/L, KH₂PO₄ 11.2 g/L, MgSO₄·7 H₂O 6.3 g/L, K₂SO₄ 4.3 g/L, Na₂SO₄ 0.35 g/L, elements solution 10 mL/L, vitamin solution 12 mL/L.
Vitamin solution: d-biotin 0.1 g/L, Ca-pantothenate 2 g/L, Nicotinic acid 2 g/L, Thiamine-HCl 2 g/L, Pyridoxine-HCl 2 g/L, 4 aminobenzoic acid 0.2 g/L, Myo-inositol 25 g/L.
Trace elements solution: Na₂-EDTA·2 H₂O 15 g/L, FeSO₄·7 H₂O 3 g/L, ZnSO₄·7 H₂O 4.5 g/L, MnCl₂·4 H₂O 5.1 g/L, CoCl₂·6 H₂O 0.32 g/L, CuSO₄·5 H₂O 0.3 g/L, Na₂MoO₄·2 H₂O 0.4 g/L, CaCl₂) 2.27 g/L, HaBO₃ 1 g/L, KI 0.1 g/L

### Results of Fed-Batch Fermentation

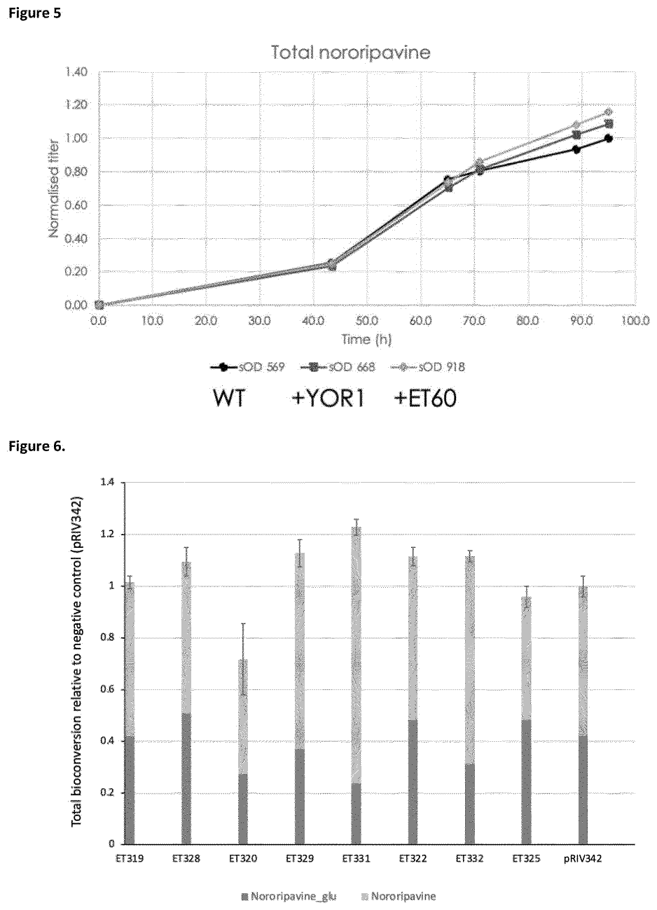

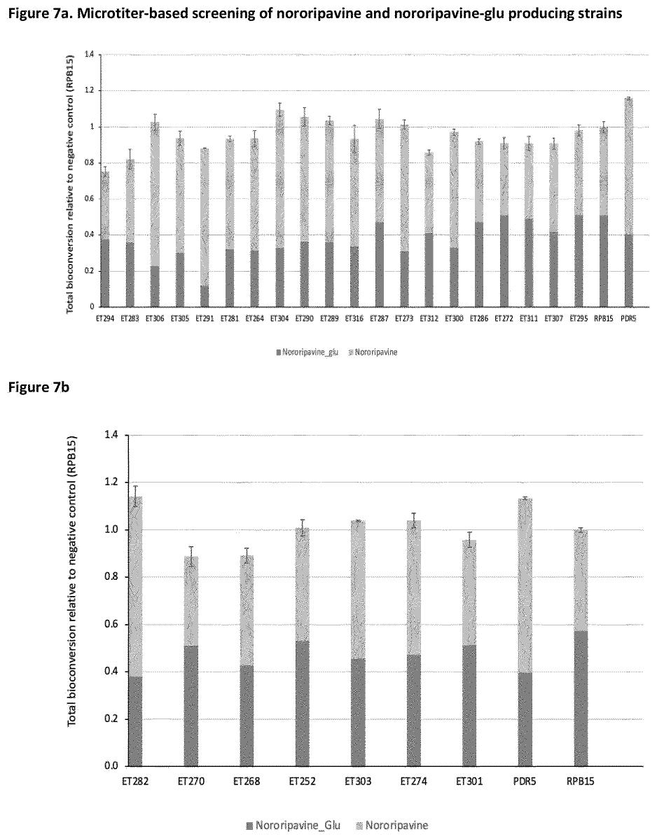

The results of the fermentations of production strains described in Example 3 are shown in FIG. 5. Here it is demonstrated that once oripavine is fed to the fermentation and the strain starts converting the oripavine to nororipavine and glucosylated nororipavine, the expression of YOR1 and YOR1 homolog ET60 ABC efflux transporters improve bioconversion of oripavine to nororipavine and nororipavine-glucoside as evidenced by increased titers and lower residual oripavine (latter not shown). All strains expressing heterologous efflux transporters (ET60, YOR1, ET71, ET58) appeared to grow better than wild type, 9-27% higher biomass by the end of fermentation with similar inoculation and early biomass levels. Additionally, it was observed in similar strains and fermentations that the total glycosylated nororipavine titer at the final timepoint was higher in strains expressing YOR1 (34%) and ET58 (9%) heterologously as compared to strains with no heterologous efflux transporter expressed.

It was also observed that ET71 and YOR1 expressing strains produced the highest percentage of glycosylated nororipavine product as compared to total nororipavine-containing product (glycosylated and non-glycosylated, data not shown).

### Example 5 Analysis of YOR1 Homologs Showing Efflux Activity on Glucosylated Nororipavine

It was noted that many of the highly active transporters identified in the microtiter plate assays had homology to the endogenous yeast transporter, YOR1, given the systematic name of YGR281W by the Saccharomyces Genome Database (https://www.yeastgenome.org).

YOR1 homologs are defined for the purposes of this example as proteins which when blasted against Saccharomyces cerevisiae S288C, result in the top hit being YOR1. More specifically, blastp (https://blast.ncbi.nlm.nih.gov/Blast.cgi?PAGE=Proteins) searches were conducted using default scoring parameters (BLOSUM 62, Existence 11, Extension 1) and selecting organism Saccharomyces cerevisiae S288c (taxid:559292) rather than using the nr (non-redundant) default database. The top hit is defined as the one with the lowest E value (displayed first in the blast output results). With the results presented here we show that fungal transporters, having homology to Saccharoyces cerevisiae S288C YOR1, as described here, are generally very likely to be useful for excreting glucosylated nororipavine.

The efflux transporters showing activity on glucosylated nororipavine, including the transporters found to have the highest specificity for glucosylated nororipavine, ET58, ET83, ET47, ET71, ET60, ET193, ET208, ET212, ET72, ET63, YOR1, and ET120, were compared to one another by pairwise sequence homology. See Table 5 below showing pairwise identity between each of the listed proteins:

TABLE 5

| | ET58 | ET83 | ET47 | ET71 | ET60 | ET193 | ET208 | ET212 | ET72 | ET63 | YOR1 | ET120 |
|---|---|---|---|---|---|---|---|---|---|---|---|---|
| ET58 | 100 | 75.95 | 64.73 | 50.88 | 46.33 | 49.43 | 48.69 | 45.13 | 47.66 | 45.68 | 45.61 | 46.96 |
| ET83 | 75.95 | 100 | 65.4 | 50.98 | 45.96 | 48.58 | 49.86 | 45.11 | 48.65 | 46.78 | 45.14 | 47.02 |
| ET47 | 64.73 | 65.4 | 100 | 50.92 | 48.14 | 48.92 | 48.3 | 44.45 | 49.68 | 48.33 | 46.23 | 47.81 |
| ET71 | 50.88 | 50.98 | 50.92 | 100 | 45.07 | 43.99 | 44.17 | 43.06 | 43.48 | 43.04 | 42.18 | 43.13 |
| ET60 | 46.33 | 45.96 | 48.14 | 45.07 | 100 | 55.53 | 52.25 | 47.66 | 47.73 | 44.79 | 43.34 | 43.57 |
| ET193 | 49.43 | 48.58 | 48.92 | 43.99 | 55.53 | 100 | 53.31 | 50.37 | 48.74 | 45.7 | 43.94 | 45.6 |
| ET208 | 48.69 | 49.86 | 48.3 | 44.17 | 52.25 | 53.31 | 100 | 48.01 | 50.68 | 50.18 | 44.67 | 45.08 |
| ET212 | 45.13 | 45.11 | 44.45 | 43.06 | 47.66 | 50.37 | 48.01 | 100 | 46.12 | 44.35 | 41.99 | 43.93 |
| ET72 | 47.66 | 48.65 | 49.68 | 43.48 | 47.73 | 48.74 | 50.68 | 46.12 | 100 | 68.56 | 44.32 | 44.3 |
| ET63 | 45.68 | 46.78 | 48.33 | 43.04 | 44.79 | 45.7 | 50.18 | 44.35 | 68.56 | 100 | 42.31 | 43.26 |
| YOR1 | 45.61 | 45.14 | 46.23 | 42.18 | 43.34 | 43.94 | 44.67 | 41.99 | 44.32 | 42.31 | 100 | 62.68 |
| ET120 | 46.96 | 47.02 | 47.81 | 43.13 | 43.57 | 45.6 | 45.08 | 43.93 | 44.3 | 43.26 | 62.68 | 100 |

The proteins described above, were further analyzed by doing a full length alignment ClustalW version 2.1 software available on genome.jp as shown in Table 6 below. Asterisks indicate absolute conservation amongst proteins, and colons or periods indicate conservative substitutions, with colons being more conservative substitutions than periods. Known motifs for transporters are designated in bold below the amino acid residues.

TABLE 6
Multiple sequence alignment of most active YOR1 homologs
CLUSTAL 2.1 multiple sequence alignment

| | |
|---|---|
| ET72 | ------------------------------------------------------------ |
| ET63 | ------------------------------------------------------------ |
| ET208 | ------------------------------------------------------------ |
| ET60 | ------------------------------------------------------------ |
| ET193 | ------------------------------------------------------------ |
| ET212 | ------------------------------------------------------------ |
| YOR1 | MTITVGDAVSETELENKSQNVVLSPKASASSDISTDVDKDTSSSWDDKSLLPTGEYIVDR |
| ET120 | ---------------MPTIRQELRHSSSGSENEKAESLYVKNEGKLDKVATQNSYYEVDR |
| ET58 | ----------------------------------------------------------MA |
| ET83 | ------------------------------------------------------------ |
| ET47 | ----------------------------------------------------------MT |
| ET71 | ------------------------------------------------------MSSNTS |
| ET72 | ---------MSVEETAGTNLALQKRSLTFLFG-KKVPPLPLEEERKVFPHYHTNIIYRAF |
| ET63 | ---------MNVEAANEKELLLQKRSLTFLYG-KNVPPLPLEEERKVEPHRHTNIISRAL |
| ET208 | ----------------MSEPPRQKRILSWALS-KKVPPITQEEDRLEYPEKRANILSKIE |
| ET60 | -MVENKANKLDSSSLEDNGLERQQRLLSFLWP-KTVPPLPNEDERLLYGEKRAGIFSKAF |
| ET193 | ---------MSDYDLEENHLVRQNRLLSSLFS-KELPPIPEDDERPEHPERDANFFSKIE |
| ET212 | --------MAELEKGDEAQPALQHRLCTPLLS-KKVPPVPRDEDRPVHP-KAINPFSWEE |
| YOR1 | NKPQTYLNSDDIEKVTESDIEPQKRLESELHSKKIPEVPQTDDERKIYPLFHTNIISNME |
| ET120 | NRPETEMNSDDLEKVTESEIYPQKRMESFLHSKKIPPIPTDE-ERPVYPLFHANWISRIE |
| ET58 | KDGIVISTEAPLKDAESGQLVLERRLLTPLLS-KKVPPIPTDEERKFYPEKKANPISKVE |
| ET83 | MAEDLGLSSPKTDPEDSSKLVLERRIMTPFLS-KKVPPVPTEAERKYFPKNKN-PLSLIE |
| ET47 | SPGSEKCIPRSDEDLERSEPQLQRRLLTPFLLSKKVPPIPKEDERKPYPYLKINPLSQIL |
| ET71 | MKDEDDYQDLEKSNAHIQQPRPVKRLLTPLLT-KYVPPIPQESERTRFPFYHTNIFSKAL |
| | .* : * : :* . : : |
| ET72 | FWYLTPLMRVGYKRTLQPEDMYLLDEEQTIDYMYKKFIASVDSDLEKQKAKHILKKCKER |
| ET63 | FYYLNPMLRVGYKRTLQPQDMYVLDERDSVDAMYDKERNYLDVELDKARAKHIQKKREAR |
| ET208 | FSWLDPLLHKGYRRTLEPEDLWYLIDELKLEHYYSVFLAQFEPDLAARREAHLEAKCKAR |
| ET60 | FWWMIPVMNPGYMRTLQPEDLFTLIDDISVEQMSARENKLFKKKVDKAKRKHIIQKFKNR |
| ET193 | FWWMIPVMNTGYKRTLTPKDLFTLSDDIKVETMAAREMAIFTSDVERAKLKHVKKKCKKR |
| ET212 | FTWLTPVLLRGYKRTLLPEDMFKLHDEMTVEHLAGKFQAIFDRRLAADKKKYLKRRRQLA |
| YOR1 | FWWVLPILRVGYKRTIQPNDLFKMDPRMSIETLYDDFEKNMIYYFEKTRKKYRKRHPEA |
| ET120 | FWWVFPILRVGYKRTLQPGDLWKMDDRMSIETLYADEERYLEVYREKARVQYRKEHPNA-- |
| ET58 | FWWLNPIMNVGYKRTLTPQDLFKLTPDMTIDHTYEKFDRYLTKIVEKDRAAALKKDPSL- |
| ET83 | YWWLNPIMKVGYKRILTPNDLYKLTPEMKIDHTYDKFEKILMKIVEKDRAKALAEDPSL- |
| ET47 | FWWLNPLLRVGYKRTLDPNDFYYLEHSQDIETTYSNYEMHLARILEKDRAKARAKDPTL- |
| ET71 | FAWLLPLLEKGYKRTLQQEDLWKLDEHTSIDHVYIKFEKHLNEEWLKEDAKHDPIK---- |
| | : :: *:: ** **: *:: : :: : . |
| ET72 | GETPES--SSVDPETDLEDFELHYVYLVKGLIRVFGWQYGWATFIKAFADLSSALLPLVL |
| ET63 | AELGNT--STVDEDTDLEDFELPYIVIVKGLFHLFGWQYMWGSLLKVFTDLFYTLMPLVQ |
| ET208 | GETFET--STVTEDEDLADFVYPWPKFGLILLKTFFRQYVGACVLKTIGDLASTTAPLLQ |
| ET60 | NEKVEI-SDIDQYKDDLEDFTPPQFLPWFVIIETFKWEYFAAVIFLALMYGTSSCIALVT |
| ET193 | GETLES-SSVD-FDTDVEDFKVSPIMFFFTIWKTYKYQYFAASVCLAIANSAQAVNPLLF |
| ET212 | LKKGDSDVSARTDEDLMLEYEPSKSLCFLSLYETFLWQYSMALLFGMLGLVGQACNPLLS |
| YOR1 | -----------TEEEVMENAKLPKHTVLRALLFTFKKQYFMSIVFAILANCTSGFNPMIT |
| ET120 | -----------TEEEIIENAVMPKHTLVKVLLYTFKWQYFLAFAAMALSNAASAFLPMVT |
| ET58 | -----------TPEDLERREYP-KFAIIKALFLTFKWEYSTAIMFKVFADVCGVCNPLLS |
| ET83 | -----------TEEDLIRRPYP-KYALPKALFLIFKWKYILALFFKVLADVCGVCNPLLS |
| ET47 | -----------TDEDLKNREYP-KNAVIKALFLTFKWKYLWSIFLKLLSDIVLVLNPLLS |
| ET71 | ----------------NPDAFP-RFAIFMALMKTFKYEYTVAIVTKIISNALSAFTPLVS |
| | : : :* . : .:: |
| ET72 | KRLINFVERKAYGLEPHVGKGIGYAFGVSLMVYFSGFAFNHFFYNSTTVGAKVKAVLTKA |
| ET63 | KRLVNFVEESAYGFHPTLGKGVGYSIGVGLMVYFAGLCVNHFVYNSITVGAKCKAVLTKL |
| ET208 | KALINYVTKRAKGLEPNVGTGVGYAIGCALFVTLEGLMVNHYFYHAMVTGSQVKAILTKE |
| ET60 | KELIKYVEYKAVGVELGIGKGLGYAFGTVGMVVFTGEMGNHYFYRAMLIGAKTKAVLIKS |
| ET193 | KKLITYVGLKAYGIEQGVGKGVGYAIGSCLIEFLGAVLENHFFYKAMMTGAETKGVLTKA |
| ET212 | RKLINFVELEALGIPIKIGTGIGYAFGVAILMEVSDVLHNQGVYEAMLIGAQIRAIFTKA |
| YOR1 | KRLIEFVEEKAIFHSMHVNKGIGYAIGACLMMFVNGLTENHFFHTSQLTGVQAKSILTKA |
| ET120 | KRLIDFVSEKSFYPGLKVNAGVGYAIGSCVMMLLNGVLENHFFHNSQLTGVQAKSVLIKA |
| ET58 | KELIKFVSRKILNADIAVNDGVGYAFGCTLLLAFSGIFINQFLHLSITTGAHCKGILTTA |
| ET83 | KKLIAFVERKTSDPSLAVNDGIGYALGCTFLVLESGIMINQSLLHSLTTGAHCKGILTTA |
| ET47 | KALINFVDEKMYNPDMSVGRGVGYAIGVTFMLGTSGILINHFLYLSLTVGAHCKAVLTTA |
| ET71 | KKLISFISEKALVPDTPINKGIGYAFGITFMLMESAIFMNQSLLHSKFVGGHSRTILTKA |
| | : *: :: :. *:**::* : . *: . : * . : :: . |
| ET72 | LLEKSFTLDARGKHKFPIGKINSIMGTDLTRVDLALGFFPELLGFPIPLIVIIVMLLVNI |
| ET63 | LLEKSFRLDARGKHKFPVGKINSIMGTDLTRVDLAIGEFPIIFEFPFSIILCIILLLVNI |
| ET208 | MLEKSFRQTGRSRHDFPTGKVNSIMGTDLARIDFAIGELPFLPCPPVPAIVSIVLLIINI |
| ET60 | ILDKSFILSPKSKLNFPHAKITSMMSTDTARIDLGLGLQPLLLIIPIPIIVSIAILIVNI |
| ET193 | LLEKSFRLSAESKHKFPVGKITSMMGTDLSRIDLALGLQPFIFVFPIPIVISIAILIVNI |
| ET212 | LLDKSFKLNTRSRKKFPPSKITSIMSTDVSRVDIGTGFSIYGFVLIFPVGVSIGILIYNI |
| YOR1 | AMKKMFNASNYARHCFPNGKVTSFVTIDLARIEFALSFQPFLAGPPAILAICIVLLIVNL |
| ET120 | ILTKSMKLSGFSRHRFPSGKITSIMSTDLSRLELAIIFQPLLGAFFVAVAICIVILIINL |
| ET58 | LLKKSFRADAETRHKFISGRITSLMSTDLARIDLAIGLQPFGWTFPIPVIIAIALLIVNI |
| ET83 | LLRKSFKADAETRHKYTSGRITSIMSTDLARIDLAIGFQPFGLTFPIPVIIAIALLIVNI |
| ET47 | IMNKSFRASAKSKHEYPSGRVTSLMSTDLARIDLAIGFQPFAITVPVPIGVAIALLIVNI |
| ET71 | LIQKSLIANAETRFHYPSGRIISFMSADLQRIDESLFELPTGFTTLEPIIIAIVLLIVNI |
| | : * : : :. .:: *:: :* *:: . : * :*: *: |
| ET72 | GVSALAGIGVFVMSIFLTGFIVRELFRLRVIANVFTDERVNLVKELLKNEKMIKMYGWEN |
| ET63 | GVSALAGISLFVVILLFTSYVVRVLYKMRVRANVYTDQRVNLVKELLKNFKMVKMYGWEN |
| ET208 | GPSSLVGIAIFFLALIALGSTIKRLMEERLRANKFTDGRVNLVKELLKNEKMIKYYSWEP |
| ET60 | GVSALTGIAVIILVLVLIMGVGYFLFKERKKANISTDQRISSIREVLYNLKIIKFYSWES |
| ET193 | GAVALIGIGVMLLEMAVIGGTTAKLYSYRTKANKYTDIRVSYMKEVINNLKMIKFYSWEP |
| ET212 | KAPAMVGVGLMLAFVFVSGGLSTLLFSERKTAQKATDSRVGYMKEVINNLKMIKFYSWEK |
| YOR1 | GPIALVGIGIFFGGFFISLFAFKLILGERIAANIFTDARVTMMREVLNNIKMIKYYTWED |
| ET120 | GPIALVGVGIFVVAMFESAYAFKRLISVRKKINIFTDARVIMMREILNSMKMIKFYAWED |
| ET58 | GVASLAGIAVFIISILVIGGSAKALLKMRRGANKFTDKRISLMREILQSMKMIKYYSWED |
| ET83 | GVSSLAGIAVFIISIVAIGGSARFLMKMRRGANKYTDKRISLMREILQSMKMIKYYSWED |
| ET47 | GVSALAGIAVELVCIVVISASSKSLLKMRKGANQYTDARISYMREILQNMRIIKFYSWED |
| ET71 | GVSALAGIAIFFLTLILMGVPAGSLFKIREAANVFTDQRVGKMREVIQSMKMIKFYSWED |
| | :: *:.::. . : * :: ** *: ::*:: .::::* * ** |
| ET72 | SYFKEFMDIRQKEMTTVLRMQVARNVLIAVAIWLPIVSSMVAFLVLHKIDS---NRTVGD |
| ET63 | SYEKQFVDTRQKEMTIVLRMQHIRNFLDALSFWLPIITSMVSFLVLYHLRN---NRTVGD |
| ET208 | SYVKNIEETRTAEMHNVELMQIMRNIMVAFAIALPTVCSMISFLVLYGINS---SRSVAD |
| ET60 | AYLKKISGIRNEETKWILRMQVLRNLIISIAISVNLICSMVSFLVLYAIDS--DRHDPAS |
| ET193 | PYYENISSTRIKEMDIIYNMQTLRSIVTALAMSLTGFASLVAFLVLEAVDN--DRKNPAS |
| ET212 | PYHALITKIRRREMAYLLRMEITRMIIITLAASLALVSSLVSFLTLYAIASP-SSRNPAE |
| YOR1 | AYEKNIQDIRTKEISKVRKMQLSRNFLIAMAMSLPSIASLVTFLAMYKVNKGG--RQPGN |
| ET120 | AYEASVHDQRSKEISKTRIMQFTRNFVTALAVCLINISSMVTFLALYKVRNHG--RIPAN |
| ET58 | AYESSVVEQRNSEVGVILKMQSIRNELLAFSISLPSFTSMIAFLVLYGISSN---RNPAN |
| ET83 | AYEKSVIQQRTKEVGIILKMQSIRNGLLAFSIALPAFTSMIAFLVLYGVSSN---KNPAN |
| ET47 | AYEKSVVTERNSEMSIILKMQSIRNFLLALSLSLPAIISMVAFLVLYGVSND---KNPGN |
| ET71 | AYENLITGIRSKESSLVLKFQLTINVMITIAINASSITSMGAFLVLYAVKSH---GNPAN |
| | .* . * * :: : :.: . *: :**.:* : . .. |
| ET72 | IFSSLSLFQELTTQFLMVPAALAMSTDMVIAFKRISQLLSCPDGQE-----------LAT |
| ET63 | IFSSLTLFQELTGQFAMVTPSLSMATDMVVGFKRVAQLISCPDAPA-----------LEE |
| ET208 | IFSSLTLFQVLAMQLIMVPLALASGSDALIGIRRVLEFVCSGDIDEE-DSQVE----LSL |
| ET60 | IFSSLTLFGILSEQVIMLPLALATTTDAHVGLQRVGQFLASEESDQTYRKIEA----SGK |
| ET193 | IFSSISLFNVLLTQVEMLPMALATSADAFAGVGRVSTFLTTGEVDPKELETDI----SAD |
| ET212 | IFSSVSLFNLLASQFLVLPLSIAGSTDAFLGMNRVAAVLAADEIDPEDADTIL----SER |
| YOR1 | IFASLSLFQVLSLQMFFLPIAIGTGIDMIIGLGRLQSLLEAPEDDPNQMIEMK------- |
| ET120 | IFSSLSLFQVLSIQMFELPMALGTAVDGSIALNRCQELFEATEEEHDIDVDFP------- |
| ET58 | IFPSISLEFGSLAQQTMMLPMALATGTDAMIGLNRVREFLQSGVDLEDPEAPQGNDQDSQD |
| ET83 | IFPSVSLFGTLAQQTMMLPMALATGADAMIGLGRVREFLESGVDLKDPEEFDG-HAPETD |
| ET47 | IFSSISLFSVLAQQTMMLPMALATGADAKIGLERLRQYLQSGDIEKEYEDHEK-----PG |
| ET71 | VFSSLSLFGILSQQVIELPMVESSAAEGLLSLDRITKYLRSPVETFDVENFYD------- |
| | :*.*::** * * :. :. : .. * . |
| ET72 | FFDTLDDPKLALQLKNASFQWFTFEDEKPEDSKSE------------------------- |
| ET63 | FHDLLDDEKLALKLAHASFKWHTFEESATTEVVIV------------------------- |
| ET208 | IKEKMESSGSVLRVVNASFEWETFDADE-EDIAST------------------------- |
| ET60 | TLGRMQENNIAVEVNNATFIWETFDVSDE----------------------------DSK |
| ET193 | VLQRMDKEDVVIEVNNASFEWEIFEDIEEKDPKKEKEEKKKAKKAAKETKKLAKQAKNSQ |
| ET212 | TQALLEEKKLAITVQDGEFEWELFDFDDEKSEEKE-------------------EHKDES |
| YOR1 | -PSPGFDPKLALKMTHCSFEWEDYELNDAIEEAKGEAKDEGKK-----------NKKKRK |
| ET120 | -PCD--DPDLALKVVNGSFEWQDFEAEENRLATLMEIEEKKKK-----------KTKSKK |
| ET58 | ANVEKLPEDVALSVKNATFIWETFDDEEDEGADKPKADTATEK----------KDSDIAT |
| ET83 | EEVKELPSDVAIEAKDATFIWEKFAEVDSE-----------------------ETSDASK |
| ET47 | DRDVVLPDNVAVELNNASFIWEKFDDADDN------------------------DGNSEK |
| ET71 | SELIKNDEIAVQIENGEFEWELFTEIKEDDEETKKQKKKDEKQRKKEL-----KKSQGGN |
| | .: . * * : |
| ET72 | -----------DEPESSDQKSEKSAGSEVVKTEFPGLLNLNLSIAKGEFVVVTGSIGSGK |
| ET63 | -----------PESKTSSSK-------DASRTEFPGLHDLTLSISRGEFIVVTGAIGSGK |
| ET208 | -----------NESVSENERKPDPSLEGLESTSFPGLNNINLDIRKGEFVVVTGLIGSGK |
| ET60 | ISDENSDESKNSSTTNSTSERNLDEEDKDNETPFKGLIDVNLTVNKGEFVVITGVIGSGK |
| ET193 | TITPSEEELSKIDSPKFTEKELSTESKSVEEKVFAGLNNINLSIKKNEFVVITGMIGSGK |
| ET212 | KKKKKKESKKKVKKSAKDVSDTSSASSNEKERKSFKLHNVNLDIRQGAFVVITGSIGSGK |
| YOR1 | DTWGKPSASTNKAKRLDNMLKDRDGPEDLEKTSFRGFKDLNFDIKKGEFIMITGPIGTGK |
| ET120 | DKAPEP--------KHEAASIKPGHLSDTERESFKGFHNLNFEVKKGELIIITGSIGTGK |
| ET58 | PATSTKDTHSDSELKNTASSTEEEGHESYTKSVFEGFHNINLDVKKGEFVIVTGAIGSGK |
| ET83 | SDKETSSTLSEGELKKTLSSEEEEGNEQYTNSVFEGFHNINLEIKKNEFIIVTGAIGSGK |
| ET47 | TKEVVVTSKSSLTDSSHIDKSTDSADGEYIKSVFEGFNNINLTIKKGEFVIITGPIGSGK |
| ET71 | GWFNKKTKTTEDSSNDEINKESSTDEDNTNQQKPFKLSNINLKISKGEFIVVTGPIGSGK |
| | : ::.: : :. ::::** **:** |
| | Walker A |
| ET72 | SSLLNAFSGFMPKTSGSVAKNGSLMLCGYPWVQNATIKENIVFGEEYDQEKYDTIVKVCS |
| ET63 | SSLLSAISGFMPKTGGSVAKNGSLLLCGYPWVQNATVRENILFGQPFDQTKYDEIVRVCS |
| ET208 | SSLLYALSGFMHRTQGHVATIGDLLLCGNPWIQNATVKDNISFGMPFDQQKYDNVIHACS |
| ET60 | SSLLSAISGLMTRTSGEVNVCGSLISCGEPWIQNETFKENILFGSDFDPDFYKEVVHACS |
| ET193 | TSLLNALSGFMKKTSGEVLVSSSLLLCGYPWIQNTTVRENIVFGSEWDEEKYNRVIFACS |
| ET212 | SSLLHALDGAMKKLSGDVYVNGSLLMCGTPWIQSASLRENILFGSTYDEKWYKEVIRACS |
| YOR1 | SSLLNAMAGSMRKTDGKVEVNGDLLMCGYPWIQNASVRDNIIFGSPFNKEKYDEVVRVCS |
| ET120 | TSLLNALAGFMRKTEGDVYKNGSLLLCGYPWVQNATVRDNILFGSPYDKARYKEVIRVCS |
| ET58 | SSLLIALAGFMKQTGGTLTAAEDVLLCGAPWVQNTTVRENITFGLPYEEERYERVIDACA |
| ET83 | SSLLTALAGFMKRTTGSLSVGGSVLLCGTPWVQNATVRENITFGLEYDEERYERVLDACA |
| ET47 | SSLLVALAGFMKKTSGTLGVNGTMLLCGQPWVQNCTVRDNILFGLEYDEARYDRVVEVCA |
| ET71 | SSLLSAISAFMTKIDGKIAINGSNLLCGAPWVQNTTIRENVLFGSKFDHVKYKKVLEVCS |
| | :*** *: . * : * : : ** **:*. :.::*: ** :: *. :: .*: |
| ET72 | LTGDFEQFSAADKTEVGERGITLSGGQKARINLARAVYSDKDIILLDDVLSAVDAKVGKS |
| ET63 | LDTDFELFSAGDMTEVGERGITLSGGQKARINLARAVYSDRDIILLDDVLSAVDAKVGKH |
| ET208 | LEADLDLLPAGDHTEVGERGITLSGGQKARLNLARAVYADRDIILLDDILSAVDARVGKH |
| ET60 | LESDMEILPAGDKTEIGERGITLSGGQKARLNLARAVYTNKDIILLDDVLSAVDARVGKH |
| ET193 | LESDIEILPGGDLTEIGERGITLSGGQKARINLARAVYGGREIILMDDVLSAVDARVGKH |
| ET212 | LESDEDILPAGDLTEIGERGITLSGGQKARVCLARTVYANSSIILLDDVLSAVDAKVGKH |
| YOR1 | LKADLDILPAGDMTEIGERGITLSGGQKARINLARSVYKKKDIYLFDDVLSAVDSRVGKH |
| ET120 | LQADLDILPANDKTEIGERGITLSGGQKARINLARSVYKSMDTYLFDDVLSAVDARVGKH |
| ET58 | LRDDLKLFAGGDLTEIGERGITLSGGQKARINLARAVYADKSIVLFDDVLSAVDARVGKH |
| ET83 | LRDDLKLFTGGDLTEIGERGITLSGGQKARINLARAVYADKEIVLFDDVLSAVDARVGKH |
| ET47 | LGDDLKMFTAGDQTEIGERGITLSGGQKARINLARAVYANKDIILLDDVLSAVDARVGKL |
| ET71 | LEHDLKSLLAGDMTEIGERGVTLSGGQKARVNLARAVYADKEVYLFDDILSAVDANVGKN |
| | * *:. : . * **:****:*********: ***:** . *:**:*****:.*** |
| | Linker Walker B |
| ET72 | IMKDCIMGYLKNKTRVLATHQLSLIDSADKIMFLNGDGTVDYGTLDEVKSRNPEFVRLME |
| ET63 | IMEQCILGYLKGKTRILATHQLSLINAADKVIFLNGNGSIDYGTLHEVRSRNSAFIRLME |
| ET208 | IMDECLLGLLKDKTRLLATHQLSLISAADRVIFLNGDGSIDVGTTAELLARNEGFTKLME |
| ET60 | IMNNCILGTLSSKTRILATHQLSLIGSADKVIFMNGDGSLEIGKFDELIQNSSGFKDLMS |
| ET193 | IMNNCILDLLKDSTRILATHQLSLIDSADRVIFLNGDGSISVGINEELQKSNPGFAALMA |
| ET212 | IMSECIMGILKGKTRVLATHQLSLISEAEHVIFLNGDGTISRGTFEELKSTNSAFKALME |
| YOR1 | IMDECLTGMLANKTRILATHQLSLIERASRVIVLGTDGQVDIGTVDELKARNQTLINLLQ |
| ET120 | IMDECMLGRLGNKTRILATHQLSLIDRASRVIFLGTDGSFDFGSVTELKKRNAGENKLME |
| ET58 | IIDDCFGEYMKGKTRVLATHQLSLVDKADRVVFLNGDGTLHIGTVEELLTSNEGFIKLME |
| ET83 | IVDDCLCDEMGHKTRILATHQLSLIDKADRVIFLNGDGTIHIGTVNELLQSNEGFVKLME |
| ET47 | IVDDCLTSFLGDKTRILATHQLSLIEAADRVIYLNGDGTIHIGTVQELLESNEGFLKLME |
| ET71 | ITENCLLGLLSSKTIIIATHQLSLISKADRVVFLNGDGLIDVGTESELRSKNKDFVKLME |
| | * .:*: : .* ::*******: *.::: :. :* . *. *: . : *: |
| ET72 | FSHNVDDDDEDENEPEQEKND-----------DFGADD---------------------- |
| ET63 | FSHDPEEEQRPD-EADEKKED-----------ELKAEK---------------------- |
| ET208 | FSTQEKNDTTTESGEAAHSGP-----------ELEDEKELIRIQTLIKSLAEAESNSDYQ |
| ET60 | LNAQEVVRDVINNVENDSKFAG----VEDEKQYIEEQLMRRITTTSYIEDEKSGRNGVNL |
| ET193 | HNAKTEEDDEDEKIDVDLDKQ-----KFEEHHEVEKELIQRQVTRASAVDEEAIRKDYNK |
| ET212 | HNRKSEENDEDESEP------------ASEVEASEKELIKRQLTKQTTTQVSS---DSVE |
| YOR1 | FSSQNSEKEDEEQEAVVAG----------ELGQLKYESEVKELTELKKKATEMSQTANSG |
| ET120 | FANKSSDKEEGELDSTEASGDDVSTAEELEHFRDDDGQREMDASRLKKELSKRSYESSVD |
| ET58 | FSKKSSEDDEEEDED-------------------IDEEEQEIIALQKSQSLAVIQSKKNN |
| ET83 | ESKKSEEDEKNEEE----------------------EEEADVLKLQKSQSIAASQ----N |
| ET47 | FSRKSESED----------------------------EEDVEAANEKDVSLQKAVSVVQE |
| ET71 | YNKELEQKNTDDEE------------------------------QINDKITKVTSIADKP |
| | . |
| ET72 | --DEDGRLIRAEEKAVNAISWDVYRTYIKAGSG-KLGYFYPVVVVLAFSVSTFCLLFVNN |
| ET63 | --EEDGKLMRDEERAVNSISKNVYSTYILSGSG-KLGYLFPILILFACAVSTESDLFTNN |
| ET208 | HKDADGVLMQLEDRAVNAIELGVYGKYLKLGAG-AFGIGIIPLLLGLVACSVFCSLFTNT |
| ET60 | DRIDDGKLFLAEERAVNRIEFKVYKNYVKYGSGIESSFCIIFLFLLFTVLATYFELFTNT |
| ET193 | NVEEDGHLIEDEDRGVNAIALDVYLTYVKLGSGKYTAWGIVPPMLVFMALATFCQIFTNT |
| ET212 | KVELDGKLYDEEEKSVNAIGWDVYGRYILTGVQGFKFNWLLFVILCLCILGTFMSLFTNN |
| YOR1 | KIVADGHTSSKEERAVNSISLKIYREYIKAAVG-KWGFIALPLYAILVVGTTFCSLFSSV |
| ET120 | ENEAAGRLMAKEERAVNSIGFDVYKNYISAGVG-KKGFVLLPEYVILLAVTTFSLLFSSV |
| ET58 | NDAAAGVLVNEEERAKNKISSKVYTEYLREGGG-ILGKFAAPIAILLLILDVFTTIFINV |
| ET83 | NDAVAGILVGEEEKAKNGISFSVYTNYLKEGGG-IFGKFAGPLTLLFLTFDVFTSIFINV |
| ET47 | QDAHAGVLIGQEERAVNGIEWDIYKEYLHEGRG-KLGIFAIPTIIMLLVLDVFTSIFVNV |
| ET71 | SGPIDGTLFGEEERAFDSIPLSLYKQYVKAGQG-MFGFTAFPLTIICIILSVFVNLFTNV |
| | * *::. : * :* *: . .: :* . |
| ET72 | WLSFWESGKFH-EPGSFYEGIYIMFGFLCLIFLIVEFLFLVYFCNMAARRFNILAFKRLL |
| ET63 | WLSFWQDKKER-KPPGEYQGIYILLGFSTLLLLTFYFALIVQFCNKAANQENTSAFQNLL |
| ET208 | WLTFWTEKKFD-RSNGFFIGIYVMFTMLTIVFMVLEFSLLVYLTNTASRLLNIYAIRRLM |
| ET60 | WLSFWISKKFPGRLDNFYIGLYVTFTELAFIFLTLEFFVLAYVTTIASRTLNLMAVKKIL |
| ET193 | WLSFWTENKFSGKDDNFYIGIYVMFTVLSFVFLALEFMSLVYMINTAAVKLNIAAVQKVL |
| ET212 | WLSFWISRKFDQPAG-FYIGFYAAFTGLAVILMVEQFCSIIFVMNRASRILNIKALGKIL |
| YOR1 | WLSYWTENKFKNRPPSFYMGLYSFFVEAAFIFMNGQFTILCAMGIMASKWLNLRAVKRIL |
| ET120 | WLSFWTEDKFKRQAG-FYMGMYIFFVFFNYFCTTGQFTLLCYLGLTASKMLNLKAVKRIL |
| ET58 | WLSFWITYKWKNRSDGFYIGFYVMFVVLNICFIASCFVLLGYISTTSARELNLKAMRRIL |
| ET83 | WLTFWITDKWSYRSQGFYIGIYVMLVLMNIVVIACCLILMGYISTTSAKSLNLKAMKRIL |
| ET47 | WLSFWISHKFKARSDGFYIGLYVMFVILSVIWITAEFVVMGYESSTAARRLNLKAMKRVL |
| ET71 | WLSFWVAQKFKNLSNGQYIGLYVMFTVLSVLFVVVELAIMGYVFTEASKTLNLKAMQKVL |
| | **::* *: : *:* : : : . :: :* *. .:: |
| ET72 | HTPMSFLDTTPMGRVLNRFTKDTDAMDNEIQDQFRQFFQPLATIVGTLILCIIYLPWFAI |
| ET63 | HAPMSFIDTTPMGRVLNRFTKDSDVLDNEIQSQFRMFIQEFSVVVGTLILCIIYLPWFAI |
| ET208 | HVPMSFMDTTPMGRILNRFTKDTDVLDNELPEQIRLLVHFIGTITGILVLCIIYLPWFAI |
| ET60 | FVPMSFMDTTPMGRIFNRFTKDTDALDNEIVEQLTVLFYFIANITGVLILCICYLPWFAI |
| ET193 | KVPMAFMDTTPMGRILNRFTKDTDVLDNEIGEQINFALFMLSNVVGIIILCIIYLPWFAI |
| ET212 | HVPMSFMDTTPMGRILNRFTKDTDILDNEIGDRVGMVVNFTFEIWGVIIMCIIYMPWFAI |
| YOR1 | HTPMSYIDTTPLGRILNRFTKDIDSLDNELTESLRLMTSQFANIVGVCVMCIVYLPWFAI |
| ET120 | HTPMSFIDTTPIGRILNRFTKDTDILDTELTESVRLFVYQTANIIGVVIMCIIYLPWFAI |
| ET58 | HAPMAYLDVTPMGRILNRFTKDTDVLDNELGEQLRLFLHPTAEVIGVIILCITYLPWFAL |
| ET83 | HSPMSFMDVTPMGRILNRFTKDTDVLDNEIGEQVRMELHPAGEVVGVVILCIIYLPWFAL |
| ET47 | HTPMHFLDVTPMGRILNRFTKDTDVLDNEIGEQARMELHPAAYVIGVLILCIIYIPWFAI |
| ET71 | HSPMSFIDTTPVGRIINRFSKDTNSLDNEIGMQLKLELHESSTIIGIIILAIIYLPWFAI |
| | ** ::*.**:**::***:**:: :*.*: : * ::.* *:****: |
| ET72 | AIPIVYALFYLISNFYLASSREIKRLEALKRSFVYSHFNESLSGMDTIKAHNSETRFLNT |
| ET63 | AIPVMIVAYYLIANFYLASSREIKRLEAVKRSEVYSHFNEALSGLDTIKAHSSSERFMET |
| ET208 | SVPILAFCYIACASYYQASAREVKRIEALQRSFVYSNFNETLQGMEVITAYKAEKRFIAR |
| ET60 | AVPPLLFLFVAIANYYQASAREIKRLEAVQRSFVYDNFNETLSGMGTIVAYKSKHRFLNK |
| ET193 | AVPFLGFMFIAVSNYYQASAREIKRLEAVSRSFVYNNFNEVLNGINTINAYKAESREVAK |
| ET212 | AVPEIVAVFIIMANFYQASGREVKRLEAVQRSHVYNNFNESLTGMPTIKAFKSIQRFLNK |
| YOR1 | AIPELLVIEVLIADHYQSSGREIKRLEAVQRSFVYNNLNEVIGGMDTIKAYRSQERFLAK |
| ET120 | AVPFLVIIFALVANHYQSSSREIKRLEAIQRSHVENNFNEVLGGIDTIRAYRGQERFLMK |
| ET58 | VIPPLLVVESCVTSYYQSSSREVKRLEAVQRSFVYNNFNEVLNGMSTLKAYRATSRFLKK |
| ET83 | VIPPVVIVSTLTASFYQASSREIKRLEAVQRSFVYNNFNEILNGMTTLKAYRSTSRFLAK |
| ET47 | AIPPLAILFTFITNEYIASSREVKRIEAIQRSLVYNNFNEVLNGLQTLKAYNATSRFMEK |
| ET71 | AVPFLAIFFLCATNFYQASSREVKRLEAINRSFVYNNFNEVMNGMNTIKAYGAGERFIRK |
| | :* : :..* :*.**:**:**:.** *:.::** : *: .: *. . **: |
| ET72 | NAHLINDMNESYYTFIAVQRWLASNLELLASLVCLLICLLCVFRVFNISGAYTGLVLTYV |
| ET63 | NSRLIDQMNESYFTEVAVQRWLACNLDIMVSEMCLFICLLCSFRIFHIRGAYAGLLLTCV |
| ET208 | NDALIDKMNEAYYLTFANMRWLSIRIDVLAAVLVLIVSLLCVMRVFHISPASVGLLLSYT |
| ET60 | NSFLIDKMNEAYYLTIANQRWLTISLDMVGAVFVLLVAMLCVNRVFHINSSSVGLLMSYI |
| ET193 | NDRLINGMNESYYLTIGNQRWLGIQMNIIAVLESLLIALLCVNRVFKISPASVGLLLSYV |
| ET212 | NVATINKMDEAYFVTVANQRWLDTYLSLLATLFALLIALLCVCRVFDIGASAVGLLVSYV |
| YOR1 | SDFLINKMNEAGYLVVVLQRWVGIFLDMVAIAFALIITLLCVTRAFPISAASVGVLLTYV |
| ET120 | NDFLTNKMNEAGYLVVAVQRWVSIALDMIAMAFALIIALLCVTRQFHISPSSVGVLLTYV |
| ET58 | NNVSVDRMNEAYFVVIANQRWISIHMDMVAVCLLFVVAMLAVTRQFSISAASAGLVVTYV |
| ET83 | NEVFVDSMNEAYFIVIANQRWISIHMDLVAVCLLAVVSLLTVTRQFNISAASTGLVVTNV |
| ET47 | NKRLLNRMNEAYLLVIANQRWISVNLDLVSCCFVFLISMLSVFRVFDINASSVGLVVTSV |
| ET71 | NDIFGDQLNEVYFVVVSNQRWIAVNLDIMATAVVFIVAMLSVTGQFSINASSVGLLTYYM |
| | . : ::* . **: *.:: . .: :* * * : *:: |
| ET72 | VNIVGLVSFMLRSMTEVENQMNSVERLKFYAIDLQQEAAYDIPERDPEPTWPASGSISFQ |
| ET63 | FNIVGMLSYMLRAMTEIENQMNSVERLKFYAVDLEQEAPFDIPERNPSPLWPQRGAICFS |
| ET208 | LNIAGMMSMLLNVSTQIENEMNSVERLEYYGFRVVQEAPFKISEKTPPPEWPHDGRIQFE |
| ET60 | LQIVGQLSFLLKTLTQVENEMNSVERICHYAFDLPEEAPYVITENSPPPSWPEKGQISFN |
| ET193 | ESIGGTLSMLIRTFTQVENEMNSVERISYYSFSLPQEAPSYITENSPPPEWPAKGEIHFK |
| ET212 | LQISGLISMLVVVFTQVEQDMNSAERVMDYVYKIPQERPYEISETRPPPEWPQNGEIRFI |
| YOR1 | LQLPGLLNTILRAMTQTENDMNSAERLVTYATELPLEASYRKPEMTPPESWPSMGEIIFE |
| ET120 | LQLPGLLNTLMRAMTQGENDMNSAERLIAYATDLPLEANYRKPEMTPAEPWPSHGEIVFD |
| ET58 | MQIGGLMSLIMRAYTTVENEMNSVERLCQYANDLVQEKPYRINETKPSPSWPESGSIEFE |
| ET83 | LQIGGLMSLIMRAYTTVENEMNSVERLYQYANTLVQEKPYRVNETVPAPSWPETGSIKFE |
| ET47 | LQIGGLMSLIMRAYTTVENEMNSVERLCHYANKLEQEAPYIMNETKPRPTWPEHGAIEFK |
| ET71 | IELSQMLSFLMQTYSEVENEMNSVERVCHYANDLEQESAYRILDYQPRPTWPEEGGIKFD |
| | ..: :. :: : *::***.**: * : * : * ** * * * |
| ET72 | DVSMRYREGLPYAVKGLSLDVAGSEKIGICGRTGAGKSSVMYSLFRLAEFEG--KITIDG |
| ET63 | NVTMSYREGLPPAVRNLSLDVAGGEKIGVCGRTGAGKSSVMYSLFRLAEFDG--RITIDD |
| ET208 | NVTLCYRQGLPAVLKNLNMDVKGAEKIGICGRTGAGKSSIMTALYRLAEMESGGRILIDD |
| ET60 | HASMAYRPELPLVLKDLDVNIKPMEKIGVCGRTGAGKSSIMMALYRLVELNSG-SVEIDG |
| ET193 | DISLAYRPGLPLVLKNLNESIKGSEKIGICGRTGAGKSSIMTALYRLSELDGG-SIVIDD |
| ET212 | NVDFAYREGLPLTLRNINADIKPHEKIGICGRTGAGKSSIMVALFRIAELTSG-SIMIDG |
| YOR1 | NVDFAYRPGLPIVLKNLNLNIKSGEKIGICGRTGAGKSTIMSALYRLNELTAG-KILIDN |
| ET120 | DVSLAYRPGLPLVLKNVSIDIGSGEKIGICGRTGAGKSTIMTALYRICELHSG-TVSIDG |
| ET58 | GVSLRYRDGLPLVLRNLTLAVAGGEKIGICGRTGAGKSSIMTALYRLSELAEG-RILIDG |
| ET83 | NVSLRYREGLPMVLKNLSMSVKGSEKIGICGRTGAGKSSIMTALYRLSELAEG-AILIDD |
| ET47 | HASMRYREGLPLVLKDLTISVKGGEKIGICGRTGAGKSTIMNALYRLTELAEG-SITIDG |
| ET71 | NLSLRYRDGLPLVLKNLSIDIKGGEKIGICGRTGAGKSSLMIALYRIAEFAEG-GIFIDG |
| | : ** ** .::.: : ****:*********::* :*:*: *: : **. |
| | Walker A |
| ET72 | VDISQIGLHKLRTKISIIPQDPVLFSGNVRSNLDPFNDRTDEELWSALEKAGLIDGSILE |
| ET63 | VDISKIGLHKLRTSLSIIPQDPVLFSGNIRSNLDPFQEHSDDNLWDALSKAGLVETDALD |
| ET208 | IDISTLGLHDLRSRLSIIPQDPVLFRGSIRGNLDPFHEHKDELLWDALRRSGLIEGSKLD |
| ET60 | IDISTLGLNNLRSRLSIIPQDPILFSGTIRINLDPFDEYTDTELWDALKRSGLIDESKIS |
| ET193 | IDISTLGLHDLRSKLSIIPQDPVMFRGTIRKNLDPFDQSTDDQLWGALVRTGLVEADRLD |
| ET212 | IDVSTLGLHELRSNLSIIPQDPVLFKGTIRSNLDPFETKTDDELWDTLRRADIIDAASLE |
| YOR1 | VDISQLGLFDLRRKLAIIPQDPVLFRGTIRKNLDPFNERTDDELWDALVRGGAIAKDDLP |
| ET120 | VDISKIGLYDLRSKLSIIPQDPVLFKGSIRRNLDPFNERTDEQLWDALVRSGAVEASEIA |
| ET58 | LDISKMGLFELRSKLSIIPQDPVLFQGTIRRNLDPFGESDDQHLWDSLRRAGLIDSSVLA |
| ET83 | VDISKLGMFELRSKLSIIPQDPVLFQGSIRKNLDPFGESDDEHLWDALRRSGLTDASILT |
| ET47 | VEISQLGLYDLRSKLAIIPQDPVLERGTIRKNLDPFGQNDDETLWDALRRSGLVEGSILN |
| ET71 | TDISKLGLHDLRSKLSIIPQDPVLFQGTIKSNLDPFNESTESELWDALRRSGLITPEEMI |
| | ::* :*: .** ::******::* *.:: ***** : **.:* : . : |
| ET72 | QVKKQQKTDAN----------------------LHKFHLDRVVEDDGSNFSLGERQLLAL |
| ET63 | LVKHQTKSDRN----------------------LHKFHLARLVEDDGSNFSLGERQLLAL |
| ET208 | QVKHQTLDDEN----------------------LHKFHLGQNVEDDGTNFSLGERQLLAL |
| ET60 | SVQSQDPKSED----------------------LNKFHLFKQVQENGTNFSLGERQLIAF |
| ET193 | VVKAQVKVQKEDKSDHGDNNNGADKKGAEEGSILHKFHLDQMVEDEGVNFSLGERQLIAF |
| ET212 | HVKTQRVGDDD----------------------FHKFHLDNEVDDEGFNFSLGEKQLVAF |
| YOR1 | EVKLQKPDENGTHGK------------------MHKFHLDQAVEEEGSNFSLGERQLLAL |
| ET120 | EVKAQSPETSGAYAN------------------MHKFHLRQEVEDDGSNFSLGERQLLAL |
| ET58 | TIKAQ----GKEDKN------------------FHKFHLDQAVEDDGSNFSLGERQLLAL |
| ET83 | TIKAQ----TKDDPN------------------FHKFHLDQIVEDEGSNFSLGERQLLAL |
| ET47 | TIKSQ----SKDDPN------------------FHKFHLDQTVEDEGANFSLGERQLIAL |
| ET71 | KMKDE----NENE--------------------YSKFHLNSVVEDEGSNFSLGERQLLAL |
| | :: : **** *:::* ******:**:*: |
| | Linker |
| ET72 | ARALVRGARILVLDEATSSVDYETDAKVQKTITEEFAQCTVLCIAHRLKTIVKYDRILVL |
| ET63 | ARALVRGSKILVLDEATSSVDYETDAKVQKTITNEFADCTVLCIAHRLKTIVKYDRIMVL |
| ET208 | ARALVRNSKILILDEATSSVDYETDSKIQTTISTEFAGCTIMCIAHRLKTIVNYDRILVL |
| ET60 | ARALVKRTKILILDEATSSVDYETDNKIQKTILKEFGTCTILCIAHRLKTIINYDRILVL |
| ET193 | ARALVRNSKILILDEATSSVDYETDAKIQNSIVNEFADCTILCIAHRLKTIINYDKILVL |
| ET212 | ARALVRDTKIIVLDEATSSVDYATDSKLQKAIVREESDRTILCIAHRLKTILHYDRVIVM |
| YOR1 | TRALVRQSKILILDEATSSVDYETDGKIQTRIVEEFGDCTILCIAHRLKTIVNYDRILVL |
| ET120 | TRALVRQSKILILDEATSSVDYETDAKIQAKIVQEESSCTILCIAHRLNTILDYDRILVL |
| ET58 | ARALVRNSRILILDEATSSVDYETDAKIQSTIKSEFSECTILCIAHRLKTILDYDKILVL |
| ET83 | ARALVRNSRILILDEATSSVDYETDAKIQNTITNEESNCTILCIAHRLKTILNYDRILVL |
| ET47 | ARALVRNSKILILDEATSSVDYETDSKIQKTISTEFSHCTILCIAHRLKTILTYDRILVL |
| ET71 | ARALVRRSKILIMDEATSSVDYKTDSLVQETIAREFSDCTILCVAHRLKTIIKYDRILVL |
| | :****: ::*:::********* ** :* * **. *::*:****:**: **:::*: |
| | Walker B |
| ET72 | DKGEIAELDTPRNLYE-QNGIFRSMCDKSGIVEDDF------ |
| ET63 | DKGEIVELGKPIELYQ-HDGIFRSMCEKSGITRADFDI---- |
| ET208 | DKGEISEEDKPWALFQDESTIFRQMCNKSGVVAEDFEKQN-- |
| ET60 | EKGEVKEFDTPWNLENTKDSIFEQMCRKSKITSDDFTIKTI- |
| ET193 | DKGEIKEFNTPWNLEKTKDSIFQQMCIKSNIVEEDFHRVSKF |
| ET212 | EQGEIKEEDTPSHLYNSTGTIFRQMCDKSGISKEDFYEW--- |
| YOR1 | EKGEVAEFDTPWTLESQEDSIFRSMCSRSGIVENDFENRS-- |
| ET120 | EQGSVAEEDTPKALFRAG-GIFTEMCQRSGITSADFKEN--- |
| ET58 | EAGEIEEFGTPMILYEND-GIFRQMCDRSDITREDFVHDL-- |
| ET83 | EQGEIEEEDTPIRLYEND-GIFKQMCERSDITREDFHIQK-- |
| ET47 | EKGEVEEEDTPRVLYSKN-GVFRQMCERSEITSADFV----- |
| ET71 | EKGELEEFDKPLDLEKKQ-GIFRDMCKISNIGVEDF------ |
| | : *.: *:..* *: :* .** * : ** |

Walker A, B and the linker regions (Katzmann, 1995; Walker, 1982) for transporters are shown in bold. Note that a high degree of conservation and conservative substitutions are present between all of the active YOR1 homologs analyzed here, near the Walker regions presumed to be involved in substrate binding and linker regions. The predicted linkers are absolutely conserved in the YOR1 homologs studied, and are regions 710-714 and 1366-1371 of the S. cerevisiae YOR1 sequence, corresponding to amino acid sequences LSGGQ and NFSLGE, respectively. The Walker A sequences are present in YOR1 at 621-627 and 1247-1253 corresponding to amino acid sequences G (S/A/L/V/M/P) IG (T/S) GK and GRTGAGK, the latter being conserved in all the YOR1 homologs shown above. Walker B sequences are found in YOR1 at 730-734 and 1387-1391, highly homologous regions with conservative substitutions amongst the YOR1 homologs above. Another observation is that the N-terminal region of >50 amino acids is not necessary for activity when these transporters are expressed in yeast.

### Example 6 Additional YOR1 Homologs Showing Efflux Activity on Glucosylated Nororipavine

ET319, ET320, ET328, ET329, ET331, ET332, ET325, and ET322 are additional YOR1 homologs and members of the ABCC/multi-drug resistance associated protein (MRP) subfamily that were tested in microtiter cultures for activity in strains producing glycosylated nororipavine, as described in Example 2. As can be seen below, ET331 has the highest total nororipavine under the conditions tested, though ET319, ET328, ET329, ET322, and ET332 all had higher production than the negative control (RPB15 plasmid only), whereas ET325 was approximately the same or slightly below the negative control. ET322 and ET328 were particularly effective at excreting glucosylated nororipavine, while ET331 appears to excrete unglycosylated nororipavine more effectively. Both types of transport are beneficial to overall production of nororipavine products. Of the 9 new YOR1 homologs tested, 8 showed similar or higher bioconversion of oripavine than the negative control; ET320 did not perform well under the conditions tested.

Table 7 below shows pairwise identity between each of the listed proteins. Similar to what has been demonstrated in Example 5, the YOR1 homologs showed sequence homology to each other as well as conserved Walker sequences.

TABLE 7
Pairwise comparison

| | ET329 | ET328 | ET332 | ET331 | ET322 | YOR1 |
|---|---|---|---|---|---|---|
| ET329 | | 83.87 | 73.80 | 58.47 | 46.02 | 42.04 |
| ET328 | 83.87 | | 73.38 | 57.13 | 46.05 | 41.75 |
| ET332 | 73.80 | 73.38 | | 57.86 | 45.19 | 41.25 |
| ET331 | 58.47 | 57.13 | 57.86 | | 44.97 | 41.09 |
| ET322 | 46.02 | 46.05 | 45.19 | 44.97 | | 41.31 |
| YOR1 | 42.04 | 41.75 | 41.25 | 41.09 | 41.31 | |

The proteins described above, were further analyzed by doing a full length multiple sequence alignment using CLC Genomics Workbench software, shown in Table 8 below. Known motifs for transporters, as described in Example 6, are designated in bold.

TABLE 8
Multiple sequence alignment of most active YOR1 homologs

| | |
|---|---|
| ET329 | M----------------------------------------------------------- |
| ET328 | M----------------------------------------------------------- |
| ET332 | M----------------------------------------------------------- |
| ET331 | M----------------------------------------------------------- |
| ET322 | M----------------------------------------------------------- |
| YOR1 | MTITVGDAVSETELENKSQN--------------------------------VVLSPKAS |
| ET329 | -----------------------------------------TELEKA--DP--PVLQK-- |
| ET328 | -----------------------------------------VELEKG--SE--PELQH-- |
| ET332 | -----------------------------------------AELEKG--DDAQPALQH-- |
| ET331 | -----------------------------------------DHESAAFSSRAPPLRQN-- |
| ET322 | -----------------------------------------DDSSLEIGNDLYKP-QK-- |
| YOR1 | ASSDISTDVDKDTSSSWDDKSLLPIGEYIVDRNKPQTYLNSDDIEKVIESDIEP--QK-- |
| ET329 | --------------------------------------RLLTPFLSKKVP---------- |
| ET328 | --------------------------------------RLLTPFLSKKVP---------- |
| ET332 | --------------------------------------RLCTPLLSKKVP---------- |
| ET331 | --------------------------------------RLLSPLFTKKVP---------- |
| ET322 | --------------------------------------RILTFLFRNKFY---------- |
| YOR1 | --------------------------------------RLFSFLHSKKIP---------- |
| ET329 | ---------------PVPLE-----D-ERPYHPKWRNPFSF-LFFTWLTPVLRRGYKRTL |
| ET328 | ---------------PVPHD-----E-DRPYHPKWRNPFSF-LEFTWLTPVLLVGYKRTL |
| ET332 | ---------------PVPRD-----E-DRPVHPKAINPFSW-FFFTWLTPVLLRGYKRTL |
| ET331 | ---------------PVPQD-----H-ERHTYPLYGNPISW-FFFTWLWPVMITGYKRIL |
| ET322 | ---------------PIPKD-----DSERKIYPEQSSNILYRVFFWWVSPLMTIGYTRIL |
| YOR1 | ---------------EVPQT-----DDERKIYPLFHTNIISNMFFWWVLPILRVGYKRTI |
| ET329 | QPEDMFKLHDQMTAEYLAGKFERIFYRRLAADKER-HLLQK---AESRGETLETSSVDSD |
| ET328 | LPEDMFKLHEGITAEHLAEKFQRIFDRRLAQDKQR-HLKEK---AKARGETLETSSVESV |
| ET332 | LPEDMFKLHDEMTVEHLAGKFQAIFDRRLAADKRK-YLKKRRQLALKKGDS-DVSARTDE |
| ET331 | EPDDLYKLNDKLKADALAARFEAIFARRLAEDKRR-HLDQTLDSSKISNSSKNSSNSPDL |
| ET322 | QPDDLWILTEDMKVEH----FYNYFVTYLQVETERAHLAHIANKCKERNESVEDSSKSRE |
| YOR1 | QPNDLFKMDPRMSIETLYDDFEKNMIYYFEKTRKKYR--------KRHPEATEEEVMENA |
| ET329 | DDFAD---YQLPKSLCFLSLYETFAWQYSLALFFGVLGMSCSTCIPLLSKELINFVSAKA |
| ET328 | DDMAD---YELSKSLCFLTLYETFARQYSLALVFATLGMSCSICIPLLSRKLINFVSEKA |
| ET332 | DLMLE---YEPSKSLCFLSLYETFLWQYSMALLFGMLGLVGQACNPLLSRKLINFVELEA |
| ET331 | DDLADLADYVPSDTLCLWSLFETFKWQYLTACFLCALAQVGWICNPLLSKKLIAYVQRKA |
| ET322 | EDLQD---FVLSPMNIATVLELTFKRQILVGLILAIFSLSGIACSPLLTKELIKFVEKRS |
| YOR1 | ---------KLPKHTVLRALLFTFKKQYFMSIVFAILANCTSGFNPMITKRLIEFVEEKA |
| ET329 | FGMDVNMGRGVGYAIGVSILIFTGDILINQGIYLSMLTGAQIRAIFTKLLLDKSFKLNTK |
| ET328 | YGFNLNMGTGVGYAIGVAILIFTGDVLINQGVYLSMITGAQIRAVFTKLLLDKSFKLNAK |
| ET332 | LGIPTKIGTGIGYAFGVAILMFVSDVLHNQGVYFAMLIGAQIRAIFTKALLDKSFKLNTR |
| ET331 | LGIESDTGKGVGYALGVSLVVFCSDILFNQMYYLSSLTGAESKAIFTKVMLDKSFRLNAR |
| ET322 | LGVHTNIGQGIGYSLGVVFMMLFSNLLFNHEMYIGQSMGALIKALLTKAVINKAFKFNAE |
| YOR1 | IFHSMHVNKGIGYAIGACLMMFVNGLTFNHFFHTSQLTGVQAKSILTKAAMKKMFNASNY |
| ET329 | SRKQFPASKITSIMSTDVSRV-DLGTGFSIYGFIFIFPVGISIGILVYNIRAPAM-VGVG |
| ET328 | SRKQFPASKITAIMSTDVSRV-DLGTGFSIYGFVFVFPVGISIGILVYNIKAPAM-VGVG |
| ET332 | SRKKEPPSKITSIMSTDVSRV-DLGTGFSIYGFVLIVPVGVSIGILIYNIKAPAM-VGVG |
| ET331 | SRRVYPVSKITSIMSTDVSRI-DLGLATAPMIIVAPVPLAISIGILIHNLKAPAL-LGIG |
| ET322 | SRHKFPQSKLISIITTDLSRV-EIAVMFQPLLLCLPIPIAIAIVILVVNIRVSAV-IGIV |
| YOR1 | ARHCFPNGKVTSFVTIDLARI-EFALSFQPFLAGFPAILAICIVLLIVNLGPIAL-VGIG |
| ET329 | LMIAFLFVAGFLSFLLFSFRQTAQKSTDARVSYMKEILNNLKMIKFYSWEIPYFKLISKI |
| ET328 | LMIAFLFVAGILGAMLFSFRKTAQKSTDARVSYMKEVLNNLKMIKFYSWEKPYFSLISKI |
| ET332 | LMLAFLFVSGGLSILLFSFRKTAQKATDSRVGYMKEVINNLKMIKFYSWEKPYHALITKI |
| ET331 | IMILFLGFAGFLGSLLFKYRKLATTQTDARVSYMKEVLNNLKMIKFYSWEKPYMAMIKAV |
| ET322 | IFIIFLGFISIGAKKLFAYRDAVSKITDKRVNEMKEILNNLKMIKFYSWEHPYHENVCRV |
| YOR1 | IFFGGFFISLFAFKLILGFRIAANIFTDARVTMMREVLNNIKMIKYYTWEDAYFKNIQDI |
| ET329 | RRR-EMAYLLRMEITRMIIITLASSLTLISSLASFLTLYGIASPSARNPADIFSSVALFN |
| ET328 | RRR-EMAYLLRMEITRMIIITLASSLTLISSLASFLTLYAIASPSSRNPADIFSSVALFN |
| ET332 | RRR-EMAYLLRMEITRMIIITLAASLALVSSLVSFLTLYAIASPSSRNPAEIFSSVSLFN |
| ET331 | REK-EMTFLLKMQVTRSIIISVAVSLSLVASFASFMLLYGTASVSKRNPASIFSSVALFN |
| ET322 | RGE-EVDMILKIQTLRNVIFSLAMTLIGICSMIAFVILYAIQG-STSSPAKMFSSVSTFE |
| YOR1 | RIK-EISKVRKMQLSRNFLIAMAMSLPSIASLVTFLAMYKV-NKGGRQPGNIFASLSLFQ |
| ET329 | MLAGQFVVLPLSLAGSTDAFLGMNRVAAVLAADEIDPNDSVHMITDSEITSMQEKKLAIS |
| ET328 | MLAGQFVVLPLSIAGSTDAFLGMNRVAAVLAADEIDPKDSVRLITDDERTAMQENKLAVS |
| ET332 | LLASQFLVLPLSIAGSTDAFLGMNRVAAVLAADEIDPEDADTILSERAQALLEEKKLAIT |
| ET331 | ILASVFINLPLAIAGATDAYIGMRRVGQYLASDE-HVEDKKRVTSETDRQLMEEKNLAIT |
| ET322 | ILGLMVFFIPQALSTTADMINGFKRIGAVLSADEEEPYEGYRELNDAS------DKRAIA |
| YOR1 | VLSLQMFFLPIAIGTGIDMIIGLGRLQSLLEAPEDDPNQMIEMKPSPGF----DPKLALK |
| ET329 | VRDCDFEWEVFN----FKEEKSEDQTKDTEELKKEKKELKQKKKEEKKANKK--SKG-SK |
| ET328 | VRDCDFEWEIFD----LKEEKTEDQTKDNKELKKEKKEMKKKKKEEKKAQKA--SK--SN |
| ET332 | VQDGEFEWELFD----FDDEKSEE----TEEHKDESKKKKMKKESKKKVKKS--TKDISD |
| ET331 | VSNANFEWEIFD----IPDE---------EKIKEEKKKQKDKEKNDKKNKKKKLSSDESS |
| ET322 | LKDASFSWDVFDDEEGEEDEADGDGDGDEEEEIEDKKDKKKAKKEKKRKEKGKDTK--SS |
| YOR1 | MTHCSFEWEDYELNDAIEEAKG--------EAKDEGKKNKKKRKDTWGKPSASTNKAKRL |
| ET329 | SPTPASEE-------------------KEAEVASFK-LHDINLDVRDGEFMVITGSIGSG |
| ET328 | SPSPEVDE-------------------KTGEVSSFK-LNNINLDVKDGEFVVITGSIGSG |
| ET332 | TSSASSNE-------------------KERK--SFK-LHNVNLDIRQGAFVVITGSIGSG |
| ET331 | HEAVTQSE-------------------KPTSAATFK-LRNIDLTIMKGEFVVVTGSIGSG |
| ET322 | FPSTSSNDIELSTIPKTSPNSKANNKGEQEEATSFPGLKNIDLTIHKGEFIVITGLVGSG |
| YOR1 | DNMLKDRD-----------------GPEDLEKTSFRGFKDLNFDIKKGEFIMITGPIGTG |
| | Walker A |
| ET329 | KSSLLYALDGTMKKNAGKLLINGSLLMCGA-PWIQNSTLRENITFGSPYDEKWYNKVVNA |
| ET328 | KSSLLHALDGTMKKNSGKLLLNGSLLMCGV-PWIQNNTLRENILFGSPYDEAWYNKVVEA |
| ET332 | KSSLLHALDGAMKKLSGDVYVNGSLLMCGT-PWIQSASLRENILFGSTYDETWYKEVIRA |
| ET331 | KSSLLLALEGSMKRNSGQVKINGSLLMCGA-PWIQSSTIRENVIFNNPYNKSWYEQVIDV |
| ET322 | KSSLLSAIAGFMSCDSGEVDINGPLLLCGA-PWIQNNTIRENIVFGKPEDQEYYDKVIYA |
| YOR1 | KSSLLNAMAGSMRKIDGKVEVNGDLLMCGY-PWIQNASVRDNIIFGSPENKEKYDEVVRV |
| ET329 | CSLDSDFDLLPAGDRTEIGERGITLSGGQKARVCLARTVYADSSIILLDDVLSAVDAKVG |
| ET328 | CSLNSDFDLLPAGDRTEIGERGITLSGGQKARVCLARTVYEDSSIILLDDVLSAVDAKVG |
| ET332 | CFLESDFDILPAGDLTEIGERGITLSGGQKARVCLARTVYANSSIILLDDVLSAVDAKVG |
| ET331 | CCMDSDLEILPAGDQTEIGERGITLSGGQKARLSLARAVYARSDIILLDDVLSAVDAKVG |
| ET322 | CALNIDLDSLEGGDYTEVGEKGITLSGGQKARINLARAVYANKEIILMDDVLSAVDARVG |
| YOR1 | CSLKADLDILPAGDMTEIGERGITLSGGQKARINLARSVYKKKDIYLFDDVLSAVDSRVG |
| | Linker Walker B |
| ET329 | RHIMSECI--LGLLKDKTVVLATHQLSLISEAESVVFLNGDGTISR-GTFDELKRINSAF |
| ET328 | KHIMNECL--LGLLKNKTRILATHQLSLISEAESVVFLNGDGSISR-GSFEELKRSNPAF |
| ET332 | KHIMSECI--MGILKGKTRVLATHQLSLISEAEHVIFLNGDGTISR-GTFEELKSTNSAF |
| ET331 | KRIVDECI--LGVLRKKTVVLATHQLSLIESADKIVFLNGDGTVDV-GTSESLRRSNEAF |
| ET322 | KHILNNCF--LGLLGSKTRVLATHQLSLIGSADRIVFLNGDGSIDV-GKMDELIARNNDF |
| YOR1 | KHIMDECL--TGMLANKTRILATHQLSLIERASRVIVLGTDGQVDI-GTVDELKARNQTL |
| ET329 | ATLM-----EHSQNNEDTEE---------DSNE-QGP--TNEKELIN------------- |
| ET328 | NTLM-----EHSRKNEDSDD---------EEEDLKGA--PDEKELIN------------- |
| ET332 | KALM-----EHNRKSEENDE---------DESEPASELEVSEKELIK------------- |
| ET331 | QKLL-----SHSTTEKYAEE---------ESSISSQTDESIKKVVVE------------- |
| ET322 | NQLM-----KFSKLEDIEEENLDVEVEEIVDEIDIGNKESKDSGEIVSVFSQNQSGIDTS |
| YOR1 | INLL-----QFSSQNSEKED--------------------EEQEAVVAGELGQLKYESEV |
| ET329 | RQL-TRQTTTQVS------EETDEKNFTE----------SDGRLIMDEERSVNAIGWDVY |
| ET328 | RQL-TRQTTTQIS------DDSNESGLPE----------GDGKLIGEEERSINAIGWDVY |
| ET332 | RQL-TKQTTTQVS------SDSVEK--VE----------LDGKLYDEEEKSVNAIGWDVY |
| ET331 | AQI-SRLTSVSST------NEKTDLQ-KQ----------NEGKLIMEEEKSVNAINADVY |
| ET322 | QEL-TRRRTRIFSKSDSDNNDSMEDNDVEYKDYNHNKDATKGKIITEEERAVNSIKFDVY |
| YOR1 | KEL-TELKKKATEMSQTANSGKI---------------VADGHTSSKEERAVNSISLKIY |
| ET329 | GKYILTGVEGFKANWLIYVVFAIT--V-LTTFLTLFTNNWLSFWISMKF----DRSDGFY |
| ET328 | GRYVLTGVDGFKLNWPVYLVFGAT--V-FTTFLTLFTNNWLSFWIQMKW----DYSDGYY |
| ET332 | GRYILTGVQGFKFNWLLFVILCLC--I-LGTFMSLFTNNWLSFWISRKF----DQSAGFY |
| ET331 | VRYIFAGIPGVKGAMIFAAVIIFS--I-LSVFFNLFTSTWLSFWVEYKWR---NRSDGFY |
| ET322 | HKYLKYGA-GKLTPWGFFTIFAVL--LTLATFCDIFTNIWLSFWIEQKFD---GKSNGFY |
| YOR1 | REYIKAAVG--KWGFIALPLYAIL--VVGTTFCSLFSSVWLSYWTENKFK---NRPPSFY |
| ET329 | GKYILTGVEGFKANWLIYVVFAIT--V-LTTFLTLFINNWLSFWISMKE----DRSDGFY |
| ET328 | GRYVLTGVDGFKLNWPVYLVFGAT--V-FTTFLILFINNWLSFWIQMKW----DYSDGYY |
| ET332 | GRYILTGVQGFKFNWLLFVILCLC--I-LGTFMSLETNNWLSFWISRKF----DQSAGFY |
| ET331 | VRYIFAGIPGVKGAMIFAAVIIES--I-LSVFFNLFTSTWLSFWVEYKWR---NRSDGFY |
| ET322 | HKYLKYGA-GKLTPWGFFTIEAVL--LILATFCDIFINIWLSFWIEQKFD---GKSNGFY |
| YOR1 | REYIKAAVG--KWGFIALPLYAIL--VVGTTFCSLFSSVWLSYWTENKFK---NRPPSFY |
| ET329 | IGLYAMFTVLAVLFMVSQFCGV-IFILNRASRILNIKAIERILHVPMSFMDTTPMGRVIN |
| ET328 | IGLYAMFTALAVMFMITQFCGV-IYILNRASRILNIKALERILHVPMAFMDTTPMGRVIN |
| ET332 | IGFYATFTGLAVILMVFQFCSI-IFVMNRASRILNIKALGKILHVPMSFMDTTPMGRILN |
| ET331 | IGFYAAFTVLALVILTFGFSGV-IYVMNLSSRTLNIRAAERILYVPMSYMNVTPMGRIIN |
| ET322 | IGFYVMFNVLWVIFLTYTFVFF-IHGTTVSSKHLNLMAIKRILHAPMSFMDTTPMGRILN |
| YOR1 | MGLYSFFVFAAFIFMNGQFTIL-CAMGIMASKWLNLRAVKRILHTPMSYIDTTPLGRILN |
| ET329 | RFTKDTDTLDNEIGDRVSMVNYFLSDLIGIIILCIIYMPWFAIAVPFIIGLFIIAATFYQ |
| ET328 | RFTKDIDTLDNEIGDRVSMVVYELSDIVGIIILCIIYMPWFAIAVPFIIGEFIILATFYQ |
| ET332 | RFTKDTDTLDNEIGDRVGMVVNETFEIWGVIIMCIIYMPWFAIAVPFIVAVFIIMANFYQ |
| ET331 | RFTKDTDVLDNEMGDRMGMIIYEASIIGGVLILCIIYLPWFAIAVPFLIVVFFGFANFYQ |
| ET322 | RFTKDTDALDNEISDNLRLFFTAIAKMIGVFILIIIYLPWFACAIPGIFVLFFLIANFYQ |
| YOR1 | RFTKDTDSLDNELTESLRLMTSQFANIVGVCVMCIVYLPWFAIAIPFLLVIFVLIADHYQ |
| ET329 | ASGREVKRLEAIQRSHVYNNFNESLSGMPTIKGFGSIGRFLQKNVSTINKMSEAYFITVA |
| ET328 | ASGREVKRLEAIQRSHVYNNFNESLIGMPTIKAFKSIGRFLEKNVKTINKMNEAYYITVA |
| ET332 | ASGREVKRLEAVQRSHVYNNFNESLTGMPTIKAFKSIQRFLNKNIATINKMDEAYFVTVA |
| ET331 | ASGREIKRLEAVQRSLVYNNFNETLTGLDTIRGYDKTDVFLSKNIRLIDKMNEAYFITVA |
| ET322 | ASNREVKRLEAILRSFVYNNVNEVLSGMNTIKAYKDESRFADLGDLLLNKANEASFVVNA |
| YOR1 | SSGREIKRLEAVQRSFVYNNLNEVLGGMDTIKAYRSQERFLAKSDFLINKMNEAGYLVVV |
| ET329 | NQRWLDVHLSMLASSFAFLISMLCVFRVF---DIGASSVGLLLSYVLQISSMISMLVVVF |
| ET328 | NQRWLDVHLSMLASSFAFLIAMLCVFRVF---NINPASVGLLLSYVLQISSTVSMLVVVF |
| ET332 | NQRWLDTYLSLLATLFALLIALLCACRVF---DIGASAVGLLVSYVLQISGLISMLVVVF |
| ET331 | NQRWLDVAVSFLATIFAIIISFLCVFRVF---KINASSVGLLLSNTLQISGIITTLVVVY |
| ET322 | NQKWIGIQLDLLAELIVLIVSLLCVNRVF---SINAAAVGLIMTYTLQVANELLNLVRTF |
| YOR1 | LQRWVGIFLDMVAIAFALIITLLCVTRAF---PISAASVGVLLTYVLQLPGLLNTILRAM |
| ET329 | TQVEQDMNSAERVIEYVYKIPQENAYQISETKPSPEWPQNGEIRFLNVDFAYREGLPLTL |
| ET328 | TQVEQDMNSAERVIEYVYKIPQEKAYEISETKPAPEWPAHGEIKFINVGFAYREGLPLTL |
| ET332 | TQVEQDMNSAERVLDYVYKIPQERPYEISETRPPPEWPQNGEIQFINVDFAYREGLPLTL |
| ET331 | TRVEQDMNSAERIIEYVDDLPQEAPYTISETTPNPSWPQQGQIDFNHVNLAYRPGLPMVL |
| ET322 | TLVENDMNSAERIIHYALKVEQEAPYKIDSSQPPPDWPQYGAVDFKNVNMKYRPGLPLVL |
| YOR1 | TQTENDMNSAERLVTYATELPLEASYRKPEMTPPESWPSMGEIIFENVDFAYRPGLPIVL |
| ET329 | KNFNADIRPHEKIGICGRTGAGKSSIMVALFRIAELTSGTIEIDGVDVRTLGLHDLRSKL |
| ET328 | KNFNVDIKPHEKIGICGRTGAGKSSIMVALFRIAELSAGSIVIDGVDISTLGLHDLRSRL |
| ET332 | RNINADIKPHEKIGICGRTGAGKSSIMVALFRIAELTSGSIMIDGIDVSTLGLHELRSNL |
| ET331 | KDFTVHIDPNEKIGICGRTGAGKSSIMVALYRMVELTSGNITIDGIDIRTLGLNNLRSKL |
| ET322 | KDFSLKIAPMEKIGICGRTGAGKSSIMTALYRISELDSGSIRIDDVDIATIGLKDLRSHL |
| YOR1 | KNLNLNIKSGEKIGICGRTGAGKSTIMSALYRLNELTAGKILIDNVDISQLGLFDLRRKL |
| | Walker A |
| ET329 | SIIPQDPVLFKGTIRKNLDPFGTKSDDELWDTLRRSDIISADKL---------------- |
| ET328 | SIIPQDPVLFKGTIRKNLDPFGTKTDEELWDTLRRADIISAETL---------------- |
| ET332 | SIIPQDPVLFKGTIRSNLDPFETKTDDELWDTLRRADIIDATSL---------------- |
| ET331 | SIIPQDPVLFQGTIRKNLDPFGSATDEQLWETLRRARIIKSEDL---------------- |
| ET322 | SIIPQDPVLFNGTIRSNLDPFGEQPDDVLWESLRRSGILTTEEVAKARAISKDSITAASG |
| YOR1 | AIIPQDPVLFRGTIRKNLDPFNERTDDELWDALVRGGAIAKDDLP--------------- |
| ET329 | ----EAVKAQKVGDD----DYNKFHLDSEVDDEGENFSLGEKQLVAFARALVRNSKILVL |
| ET328 | ----EEVKAQKPGDD----DFNKFHLDGEVDDEGENFSLGERQLVAFARALVRNTKILVL |
| ET332 | ----EHVKTQRVGDD----DFHKFHLDNEVDDEGENFSLGEKQLVAFARALVRDTKIIVL |
| ET331 | ----NEVKSQ-TDPN----KMHKFHLDRDVDVDGENFSLGEKQLIAFARALVRGSKILIL |
| ET322 | GKEGSEVESQEV-------ELPKFHLYQPVEDEGENFSLGERQLISFARALVRNAKIIIL |
| YOR1 | -----EVKLQKPDENGTHGKMHKFHLDQAVEEEGSNFSLGERQLLALTRALVRQSKILIL |
| | Linker Walker B |
| ET329 | DEATSSVDYATDSKLQKAIAREFADCTILCIAHRLKTILNYDRVMVMDQGEIKEFDTPRN |
| ET328 | DEATSSVDYATDSKLQKAIAREFSGCTILCIAHRLKTILNYDRIMVMDQGSISEFDTPTN |
| ET332 | DEATSSVDYATDSKLQKAIVREFSDRTILCIAHRLKTILHYDRVIVMEQGEIKEFDTPSH |
| ET331 | DEATSSVDYATDKILQEAIVEEFSDCTILCIAHRLKTILNYDRVMVMDQGQVVEFDKPIN |
| ET322 | DEATSSVDYGTDDKIQTTIAQEFKSCTILCIAHRLKTIINYDKILVMDKGSVREFDTPWN |
| YOR1 | DEATSSVDYETDGKIQTRIVEEFGDCTILCIAHRLKTIVNYDRILVLEKGEVAEFDTPWT |
| ET329 | LFNSRNTIFRQMCDKSGISTSDFGA--- |
| ET328 | LFNSTSSLFRQMCDKSGISQFDFEE--- |
| ET332 | LYNSTGTIFRQMCDKSGISKEDFYEW-- |
| ET331 | LFKKQGTFF-QMCEKAGINEKEFGH--- |
| ET322 | LFNSNGSVFREMCEKSNIVAEDFKRR-- |
| YOR1 | LFSQEDSIFRSMCSRSGIVENDFENRS* |

In this case, the first part of the Walker A sequences have some conservative substitutions at the third and fifth amino acid residues in addition to the previous wobble allowed at position 2 resulting in a motif of: G(X)(I/V)G(S/T)GK (where X=P, L, S, A, V, M).

The second Walker A sequence is absolutely conserved as before, as GRTGAGK. The first linker region is still absolutely conserved as LSGGQ. The second linker region is also absolutely conserved as NFSLGE. The Walker B sequences observed with the new YOR1 homologs in Table 8 and the previous ones from example 5 are (I/V/T)(I/Y/V)L(M/F/L)D and I(I/L)(I/V)(L/M)D with conservative hydrophobic substitutions at the residues in parentheses.

### Example 7 Another Class of ABC Transporters for Efflux of Nororipavine and Glucosylated Nororipavine

PDR5, like YOR1, is an ABC transporter involved in drug/xenobiotic efflux. PDR5 belongs to ABCG/pleiotropic drug resistance (PDR) subfamily of ABC transporters and YOR1 belongs to ABCC/multi-drug resistance associated protein (MRP) subfamily (Kumari 2021).

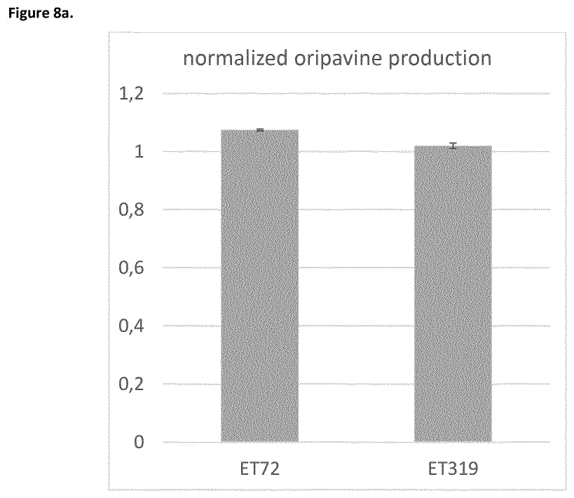

Overexpression of S. cerevisiae PDR5, was observed to increase overall conversion of oripavine into nororipavine products, although excretion of glucosylated nororipavine did not appear to be improved by expression of PDR5 as compared to a plasmid only control. Not being bound by theory, it was hypothesized that PDR5 benefits production of BIAs such as nororipavine and glucosylated nororipavine by excreting the BIA (in this example nororipavine aglycone) more efficiently and not taking it back into the cell very efficiently. Additional PDR5 homologs that are members of the ABCG/pleiotropic drug resistance (PDR) subfamily of ABC transporters, were also tested in microtiter plates as described in Example 2, at pH 5.5. As can be seen below in FIGS. 7a and b, in some cases the concentration of glucosylated nororipavine is actually lower than the no transporter (plasmid RPB15) control, which is consistent with nororipavine excretion being efficient and lower levels of nororipavine being available inside the cell for glucosylation. For example see the ET291 data below. ET291 appears to be an efficient oripavine excreter as well, which makes the total bioconversion lower than the control (plasmid only). PDR5 and ET304 performed the best, under the conditions tested, though several other transporters also appeared to have bioconversion activity above that of the control such as ET289, ET290, and ET287.

Similar assays in microtiter plates were conducted again using strains transformed with empty plasmid RPB15 as a negative control, and several more PDR5 homologs (in addition to PDR5 as a positive control). (see FIG. 7b). Under the conditions tested, ET282 and PDR5 had the highest level of bioconversion, with an improvement of over 10 percent compared to the no transporter control. ET303, ET252 and ET274 had activity at the same level or slightly higher than the negative control; and ET270, ET301 and ET268 appeared to have lower activity than the negative control under the conditions assayed, for production of nororipavine and nororipavine-glu.

A third set of assays was conducted in a similar manner as above, in which ET265 and ET299 transporters appeared to have higher activity than PDR5 for bioconversion of oripavine to nororipavine-containing products, and ET293 had higher activity than YOR1, but lower activity than PDR5, for total bioconversion or oripavine to nororipavine-containing products.

### Example 8 Sequence Alignment PDR 5 Homologs

PDR5 and homologs that showed higher bioconversion of oripavine to nororipavine+nororipavine-gly and/or better excretion of product were compared by NCBI's blastp program choosing the ‘align two or more sequences’ function. PDR homologs ET306, ET304, ET299, ET293, ET289, ET287, ET291, ET290, ET282, and ET265 were between 56.48-75.48% identical when compared to PDR5, over >97% of sequence of the PDR5 sequence length. See Table 9 below, a pairwise comparison of the full length proteins. The PDR5 homologs with desirable enzymatic properties are all >50% identical.

TABLE 9

| | ET287 | ET289 | ET290 | ET291 | ET293 | ET265 | PDR5 | ET282 | ET299 | ET304 | ET306 |
|---|---|---|---|---|---|---|---|---|---|---|---|
| ET287 | | 83.70 | 78.91 | 75.82 | 70.52 | 61.86 | 62.85 | 61.47 | 62.66 | 54.67 | 52.65 |
| ET289 | 83.70 | | 79.64 | 77.98 | 71.33 | 63.03 | 63.81 | 61.76 | 63.18 | 54.85 | 52.61 |
| ET290 | 78.91 | 79.64 | | 79.12 | 72.38 | 62.65 | 64.12 | 61.97 | 63.01 | 56.15 | 53.21 |
| ET291 | 75.82 | 77.98 | 79.12 | | 71.85 | 61.77 | 63.48 | 60.97 | 62.49 | 54.39 | 52.25 |
| ET293 | 70.52 | 71.33 | 72.38 | 71.85 | | 64.90 | 64.99 | 62.11 | 63.99 | 55.65 | 53.92 |
| ET265 | 61.86 | 63.03 | 62.65 | 61.77 | 64.90 | | 75.33 | 69.52 | 61.52 | 54.83 | 54.30 |
| PDR5 | 62.85 | 63.81 | 64.12 | 63.48 | 64.99 | 75.33 | | 69.70 | 62.34 | 54.96 | 52.64 |
| ET282 | 61.47 | 61.76 | 61.97 | 60.97 | 62.11 | 69.52 | 69.70 | | 59.23 | 53.18 | 51.39 |
| ET299 | 62.66 | 63.18 | 63.01 | 62.49 | 63.99 | 61.52 | 62.34 | 59.23 | | 55.94 | 53.90 |
| ET304 | 54.67 | 54.85 | 56.15 | 54.39 | 55.65 | 54.83 | 54.96 | 53.18 | 55.94 | | 68.77 |
| ET306 | 52.65 | 52.61 | 53.21 | 52.25 | 53.92 | 54.30 | 52.64 | 51.39 | 53.90 | 68.77 | |

Multiple sequence analysis using Clustal O v 1.2.4 (ebi.ac.uk) was performed as well, to look for conserved functional regions of the proteins with desirable properties. Table 10 shows the conserved regions of the sequence (asterisk is absolute conservation, two dots indicates highly conservative substitutions, and one dot indicates a conservative substitution).

TABLE 10
CLUSTAL O(1.2.4) multiple sequence alignment

| | | |
|---|---|---|
| ET306 | -------------------------MSNSAYDIEDHPEEVNKYDGYNNAVDSEVQRLARQ | 35 |
| ET304 | -----------MTEN----------IHLGDQVSKTSAQSVEEYKGFDSNVDDNIQHLARK | 39 |
| ET299 | -------------------------------MDNESTDSLVEYQG-DNKVENQIRDLART | 29 |
| ET293 | --------------------------------MSQEHSDQSL---YDGPNKKEIRDLART | 25 |
| ET289 | ----MSLGTTHLPEALAAN----HEAAAATSNNSEPDSFHPEYRGFEDQAQDHVRNLART | 52 |
| ET287 | ----MSLGTSHVPEALAAE----LEAVR---NRDESSSTAEKYEGLDSHAKDSVRDLARS | 49 |
| ET291 | ----MKANKSNMA---GDP----HGSCDSAVLTRNTSPELPEYDGLDCSARASVRELART | 49 |
| ET290 | MVDNVAPNGASYQ---NNN----IGNEMAADDISEQASSPGVYRGFEgAQQSVRNLARS | 53 |
| ET282 | -----------------------MVVG-SVEQHPGGYSDENAYGGFDEAINGSVKELARS | 36 |
| PDR5 | -----------MPEAKLNNNVNDVTSYSSASSSTENAADLHNYNGFDEHTEARIQKLART | 49 |
| ET265 | -----------MSEK-----KDIIDSGAMSQVDESSSLNLQSYDGFDQNAQDKIRQLARS | 44 |
| | : :: *** | |
| ET306 | ITQNSQLSFQDDG------FKLAPGESNIDGLSRVSTIAPGVNPMQNVEELDPRLDPNSE | 89 |
| ET304 | LTNASQSSLPAAAGEDQQYYQNNNDLEAQQSLSRVSTIAPGVVAINN-PDIDPRLDPNSD | 98 |
| ET299 | LSRASLKEQGSNH---DLVSGAADDDTRSIFSTKYEGVNP-VFSDANAPGYDARLDPNSD | 85 |
| ET293 | LTAASVAV----------------------SGNSDAAVNP-LAAQPGEPGYNARLDPNSD | 62 |
| ET289 | LTNASAPG----------------------SVASMSPINP-VFSTPDAPHYDARLDPNSD | 89 |
| ET287 | LTHEGAAA----------------------ADGSESPVNP-VFSDPNAPDYDARLDPNSD | 86 |
| ET291 | FTNGSVASNSKAAAVGVHATTALPASRITTEYGDVNTTNP-VFSSSELPSYNSRLDPNSD | 108 |
| ET290 | LTNRSGD--------------------AVSMAADWNTINP-VFSTVESPSYDPRLDPNSD | 92 |
| ET282 | LIEENKDSTNSF-------------SERENTVENNTGINP-VGLQPSDAEYKPELDPSSN | 82 |
| PDR5 | LTAQSMQNSTQSAPNKS--------DAQSIFSSGVEGVNP-IFSDPEAPGYDPKLDPNSE | 100 |
| ET265 | LTNQSSTTH------NN--------DAA-SLFSNVKGVNP-VFTDPSNPEYDERLDPESE | 88 |
| | : . * : . .***.*: | |
| ET306 | EFQSRYWIKNFKALMDKDPDHYKNYSLGVTEKNLRASGEASDADYQTTIINAPFKIAKQY | 149 |
| ET304 | QFNSRFWVKNFKNLMDKDPEHYKSYSLGIAYKNLRATGEAAGADYQTTVMNAPLKYANLA | 158 |
| ET299 | NFSSAAWIRNMVALAGCDPEYYKHYTISCCWKDLRAFGDPTDVAYQSTMVNMPQKIESQV | 145 |
| ET293 | EFSSEAWVRNLSHLTAENPDYYKPFSLGCAWKNLRAYGDSTDVAYQSTVANLPWKLLQFG | 122 |
| ET289 | EFSSAFWVHNVSQLVARDPDHYKPYSLGCSWRNLRAYGNATDVAYQSTVANLPLQLAETV | 149 |
| ET287 | EFSSAFWVRNLSELVRQDPDHYKPYSLGCSWRNLRAYGNATDVAYQSTVVNIPVQLVETV | 146 |
| ET291 | EFSSALWVKNLSQLIASDPDHYKPYSLGCTWKNLRAYGNATDVAYQSTFANLPLQLLESG | 168 |
| ET290 | EFSSSLWVQNLSQLVSSNPDHYKPYSLGCSWRNLRAYGNSTDVAYQSTVVNLPLQLVEYV | 152 |
| ET282 | DFSSKAWIGNLAKVVVSDPDYYKPYTVGCGWRNLSALGASADVAYQTTVDNMPWKILSWL | 142 |
| PDR5 | NFSSAAWVKNMAHLSAADPDFYKPYSLGCAWKNLSASGASADVAYQSTVVNIPYKILKSG | 160 |
| ET265 | NFSSTAWVKNMANLTAADPDYYKPYSLGCVWKDLTASGDSSDVVYQSTVFNMPTKLLKTA | 148 |
| | :*.* *: *. : :*:.** :::. :::* * * :.. **:*. : : : . | |
| ET306 | AKAVFSTRSAKQANRFDILKSLDGIVRPGEVLVVLGRPGSGCTTYLKSIASNTHGFKIGQ | 209 |
| ET304 | KKAFFTSKAKKEAGRFDILKSMDALVRPGEVVVVLGRPGSGCSTLLKTIASNTHGFAIGE | 218 |
| ET299 | KR---SLCKPKDEQVFDILKPMDGLLKAGELLVVLGRPGSGCSTLLKTISANVQGYHLDE | 202 |
| ET293 | YR---CVRKSRPSDKFDILKSMDGILNPGELLVVLGRPGSGCSTLLKSISSNTHGFKVAP | 179 |
| ET289 | LR---MARKARPEDTFDILKPMDGLVKPRELLVVLGRPGSGCSTLLKTVSANTHGFHVDP | 206 |
| ET287 | YR---MARKARPEDTFNILKPMDGLVKPRELLVVLGRPGSGCSTLLKTVSANTHGFHVDP | 203 |
| ET291 | YR---AARKARPEDSFDILKPMDGIVKPSELLVVLGRPGSGCSTLLKSISANTHGFHIDS | 225 |
| ET290 | YR---AARKARPEDTFDILKPMDGMVKPGELLVVLGRPGSGCSTLLKSISSNTHGFHIDK | 209 |
| ET282 | YR---MARPAKESDTFEILKPMDGLVKPGELLVVLGRPGSGCTTLLKSISSNTHGFKIPK | 199 |
| PDR5 | LR---KFQRSKETNTFQILKPMDGCLNPGELLVVLGRPGSGCTILLKSISSNTHGFDLGA | 217 |
| ET265 | FR---KARPAKESDTFQILKPMEGCINPGELLVVLGRPGSGCTILLKSISSNTHGFNVGK | 205 |
| | : : *:*** ::. :. *::**********:* **::::*.:*: : | |
| | Walker A | |
| ET306 | ESEMSYEGLTQKEIKKHFRGEVVYNAESDIHFPHLTVWQTLTTAAKFRTPENRIPGISRE | 269 |
| ET304 | EAEISYEGLSPKDIRKHYRGEVVYNAESDIHFPHLTVWQTLSTVAKFRTPQNRIPGISRE | 278 |
| ET299 | KSIVSYNGLDAKTIGKHYRGEVVYNAESDVHFPHLSVFETLYNIALLVTPSNRVKGVSRE | 262 |
| ET293 | ESEIRYDGLTPKEIAKHYRGQVVYNAESDVHFPHLSVFDTLKTIALLSTPANRIEGMDRE | 239 |
| ET289 | KSQISYDGLTPKDVARHYRGEVVYNAESDVHFPHLTVFDTLKIVARLSCPSNRIHGVDRE | 266 |
| ET287 | ESRISYDGLSPKEVAKHYRGEVVYNAESDVHFPHLTVFQTLKTVARLACPSNRIHGVDRE | 263 |
| ET291 | DSEIFYDGMDPKEIAKHYRGEVVYNAESDVHFPHLTVFDTLKTVARLSCPSNRFHGVDRE | 285 |
| ET290 | ESEISYDGLSPKEISRHYRGEVVYNAEADVHFPHLSVFDTLKTVARLACPINRIQGVDRE | 269 |
| ET282 | DATISYSGLSPKDIINHFRGEVVYCPEADIHLPHLTVFQTLLTVARLKTPRNRIRGVSRE | 259 |
| PDR5 | DTKISYSGYSGDDIKKHFRGEVVYNAEADVHLPHLTVFETLVTVARLKTPQNRIKGVDRE | 277 |
| ET265 | DSTISYNGLIPKAINRHYRGEVVYNAESDVHLPHLTVFETLYTVARLKTPSNRVQGVDRD | 265 |
| | .: : *.* . : .*:**:*** *:*:*:***:*::** . * : * **. *:.*: | |
| ET306 | DYANALTEVFMATYGLSHTKNTKVGSELVRGVSGGERKRVSIAEVSLAGARLQCWDNATR | 329 |
| ET304 | DYANHLTEVYMATYGLSHTKNTKVGNENVRGVSGGERKRVSIAEVSLSGARLQCWDNATR | 338 |
| ET299 | DFAKHVTEVAMATYGLSHTKDTKVGNELVRGVSGGERKRVSIAEVTICGSRFQCWDNATR | 322 |
| ET293 | TYAKHVTEVYMATYGLSHTRNTKVGNDLVRGVSGGERKRVSIAEVAICGSKLQCWDNATR | 299 |
| ET289 | TFSTHITEVAMATYGLSHTRNTKVGNELVRGVSGGERKRVSIAEVSICGSKFQCWDNATR | 326 |
| ET287 | AFSTHITEVAMATYGLSHTRNTKVGNELVRGVSGGERKRVSIAEVSICGSKFQCWDNATR | 323 |
| ET291 | TFATHITEVAMATYGLSHTRNTKVGSELVRGVSGGERKRVSIAEVSICGSKFQCWDNATR | 345 |
| ET290 | TFATHITEVAMATYGLSHTRNTKVGNELVRGVSGGERKRVSIAEVSICGSKFQCWDNATR | 329 |
| ET282 | AWARHVTEVIMATYGLSHTRNTKVGNELVRGVSGGERKRVSIAEVTICGSKFQCWDNATR | 319 |
| PDR5 | SYANHLAEVAMATYGLSHTRNTKVGNDIVRGVSGGERKRVSIAEVSICGSKFQCWDNATR | 337 |
| ET265 | TYAKHLTDVTMATYGLSHTRNTKVGNDLVRGVSGGERKRVSIAEVTICGSKFQCWDNATR | 325 |
| | :: :::* *********::****.: *****************::.*:::******** | |
| | S-region Walker B | |
| ET306 | GLDAATALEFIRALRTSADVLDTTALIAIYQCSQEAYDLFDKVSVLYEGYQIFFGRADKA | 389 |
| ET304 | GLDAATALEFIRALRTQADVLDTTAFVAIYQCSQDAYDLFDKVTVLYEGHQIYFGRGDEA | 398 |
| ET299 | GLDSATALEFIRALKTSTAISGSTGVIAIYQCSQDAYDLFDKVCVLHEGYQIYYGSAKEA | 382 |
| ET293 | GLDSATALEFIKALRVNAQMINTSGVIAIYQCSQDAFDLFDKVCVLHEGYQIYYGPASEA | 359 |
| ET289 | GLDSATALEFIRALRTEAKLTHSAAVIAIYQCSQDAYDLFDKVSVLHEGYQIYFGPANQA | 386 |
| ET287 | GLDSATALEFIRALRTQAKLTASAAVIAIYQCSQDAYDLFDKVSVLHEGYQIYFGPAKDA | 383 |
| ET291 | GLDSATALEFIRALRTTAKLNNSAGVIAIYQCSQDAYDLFDKVCVLHEGYQIYFGPANEA | 405 |
| ET290 | GLDSATALEFIRALRTQAKMTRSSAVIAIYQCSQDAYDLFDKVSVLHEGYQIYFGRAKEA | 389 |
| ET282 | GLDAATALEFVRALKTQTEIVHSAGCVAIYQCSQDAYDLFDKVCVLHGGYQIFFGPATKA | 379 |
| PDR5 | GLDSATALEFIRALKTQADISNTSATVAIYQCSQDAYDLFNKVCVLDDGYQIYYGPADKA | 397 |
| ET265 | GLDSATALEFIRALKTQATLINTAATIAIYQCSQDAYDLFDKVCVLYGGYQIFYGSAQKA | 385 |
| | ***:******::**:. : : ::. :*******:*:***:** ** *:**::* . .* | |
| ET306 | KEYFINMGWECPPRQTTADFLTSVTSPRERVPRAGFE---KKVPRTPSEFATYWKASPEY | 446 |
| ET304 | REYFIKMGWYCPQRQTTADFLTSVTSPRERVPQEGFE---NKVPKTPQEFETYWKNSPEY | 455 |
| ET299 | KGYFERMGYESPSRQTTADFLTAVINPAERIPNEAFVKEGRYIPSTAKEMEEYWRNSPEY | 442 |
| ET293 | KQYFEDMGYVSPERQTTADFLTAVINPAERIMNQEFIKQNKFIPRTAEEMEKHWRNSSNY | 419 |
| ET289 | KQYFETMGYVCPSRQTTADFLTAVINPAERIV-----REGCRPPATAVEMEKYWKQSPDY | 441 |
| ET287 | KAYFERMGYVCAPRQTTADFVTAVINPAERQV-----RAGARPPASAVDMETYWKQSPEY | 438 |
| ET291 | KQYFLDMGYVSPDRQTTADFLTAVTNPAERIVNQEMVQAGKVVPSSASEMEAHWKQSENY | 465 |
| ET290 | KQYFQDMGYVCPDRQTTADFLTAVINPAERILNEDMVKAGKKIPLTAAEMEAHWKQSEAY | 449 |
| ET282 | KSYFERMGYYCPSRQTTADFLTSVISPAERIINREFTEKGIAVPQTAEEMSDYWRNSPEY | 439 |
| PDR5 | KKYFEDMGYVCPSRQTTADFLTSVTSPSERTLNKDMLKKGIHIPQTPKEMNDYWVKSPNY | 457 |
| ET265 | KKYFETMGYQCPERQTTADFLTSVTSPAERVINPDFIGRGIQVPQTPEDMNNYWRNSPEY | 445 |
| | : ** **: . *******:*:**.* ** * : :: :* * * | |
| ET306 | KALIAEIDESLAANQKSELKDLIYDAKASRQSKRMRKTDPYTVSISLQTKYLLEREVYRI | 506 |
| ET304 | AKLIKDIDSEFKHQHEQNSKGLVKEAHNKKQAKHIRPTSSYTVSFWMQTRYLLTRDFQRI | 515 |
| ET299 | AALRQEIEAELSKDSTE-ARQELLDAHVARQSKRQRKSSPYIVNEGMQVKYLTMRNFLRI | 501 |
| ET293 | KRLIGQIDECFARDSDK-AKQELQDAHTAKQSKRSRPSSPYTVSYGMQVKYLLRRNIQRI | 478 |
| ET289 | KRLLGEIDEYAAHEQAD-NMRNLAENHVAKQSRRARPKSPYTVSYGLQVKYLLIRNMQRI | 500 |
| ET287 | AQLLQDIDEYEASGG-N-SKEQLAANHVAKQSRRARAASPYTASFWLQVKYLLIRNMQRI | 496 |
| ET291 | KRLINEIDHYTTHDQTG-NREQLRNAHIAKQSKRARHSSSYTVSYGLQVKYLLIRNMQRI | 524 |
| ET290 | KGLLQEIDYYSTHDQAN-NRQQLKEAHVAKQSKKARAQSPYTVSYGLQVKYLLIRNMQRI | 508 |
| ET282 | QELIAEVDETISQDHEK-SLEVIQESHNARQSKRARRAEPYTVSYFMQVKYLMIRNFWRI | 498 |
| PDR5 | KELMKEVDQRLLNDDEA-SREAIKEAHIAKQSKRARPSSPYTVSYMMQVKYLLIRNMWRL | 516 |
| ET265 | KELINEIDTHLANNQDE-SRNSIKEAHIAKQSNRARPGSPYTVNYGMQVKYLLTRNVWRI | 504 |
| | * ::: : : :*:.: * . * .. :*.:** *:. *: | |
| ET306 | KNNFGFHGFSAIANSLMALVLASIFYNMSK--TTESFYSRGAAMFFACLFNGFQSFLEIL | 564 |
| ET304 | WNDFGENSFQVFANSFMALILSSIFYNLPK--TIDSFYYRGAAMFFAVLFNGFSSFLEIM | 573 |
| ET299 | KKSYGITVGTIAGNTAMALVLGSIFYKSMQDTTTATFFYRGAAMFIAVLFNAFASMLEIF | 561 |
| ET293 | RSDAGVTIFQVVGNAAMAFILGSMFYKILKHDDTAGFYSRSGALFFAVLFNAFSCLLEIF | 538 |
| ET289 | RSNMGVTVFQVIGNGSMAFILGSMFYKVLKHDTTEGFYSRAGALFFAVLFNAFSCLLEIF | 560 |
| ET287 | RSNMGVTAFQVIGNGSMAFILGSMFYKILKHDNTAGFYSRAGALFFAVLFNAFSCMLEIL | 556 |
| ET291 | RSSMGVTLFQVIGNGGMAFILGSMFYKILKHDTTAGFYSRAGALFFAVLFNAFSCLLEIL | 584 |
| ET290 | RNNMGITLFQVIGNGGMSFILGSMFYKALKHDDTAGFYSRAGALFFAVLFNAFSCMLEIL | 568 |
| ET282 | INSSSITVFQIIGNSVMALLLGSMFYKVLKKSSTGTFYYRGAAMFFAILFNAFSSLLEIF | 558 |
| PDR5 | RNNIGFTLFMILGNCSMALILGSMFFKIMKKGDTSTFYFRGSAMFFAILFNAFSSLLEIF | 576 |
| ET265 | KNNSSVQLFMIFGNCGMAFILGSMFYKVMKHDSTSTFYYRGAAMFFAILFNAFSCLLEIF | 564 |
| | .. .. .* *:::*.*:*:: : * *: *..*:*:* ***.* .:***: | |
| ET306 | SLFEARPIIEKHKQYALYHPAAEALASVISQLPFKAFSSLMFNLIYYFMVNFRRDPGREF | 624 |
| ET304 | TLFEARPIIEKHKQYSLYHPSANALSSVLSQLPAKIFTSIAFNLVFYFMVNFRRNPGRFF | 633 |
| ET299 | SLYEARPIIEKHRRYSLYHPSADALASMLSELPSKIVTAICFNLILYFMVNFRREPGPFF | 621 |
| ET293 | ALYEARPISEKHKRYSLYHPSADALASVISEIPAKIVTAICFNIALYFLCNLRQSAGAFF | 598 |
| ET289 | ALYEARPVSEKHKRYSLYHPSADAMASVISEVPAKLLTSVSFNLALYFLCNFKREAGAFF | 620 |
| ET287 | ALYEARPISEKHKRYSLYHPAADAMASVISEIPAKLVTSVAFNLALYFLCNFKREAGAFF | 616 |
| ET291 | ALYEARPISEKHKRYSLYHPSADALASVISEIPSKLVTSVVFNLALYFLCNFKREAGAFF | 644 |
| ET290 | ALYEARPISEKHKRYSLYHPSADAMASVISEIPAKLLTSLTFNLAMYFLCNFKREAGAFF | 628 |
| ET282 | SLYEARPVTEKHKTYSLYRPSADAFASVLSEIPAKVLTAVCFNIAFYFLVNFRRDAGRFF | 618 |
| PDR5 | SLYEARPITEKHRTYSLYHPSADAFASVLSEIPSKLIIAVCFNIIFYFLVDFRRNGGVFF | 636 |
| ET265 | SLYEARPITEKHRSYSLYHPSADAFASIFSEIPTKIIIAIGFNIIYYFLVNFERNGGVFF | 624 |
| | :*:****: ***: *:**:*:*:*::*::*::* * . :: **: **: ::.:. * ** | |
| ET306 | FYLLANVISTFTMSHFFRLIGSMSSTLPQALVPGHIVLLGLSMFVGFTIPVNYMLGWCRW | 684 |
| ET304 | FYYLVNLTATFSMSHLFRLVGSAATSLPEALVPAQVLLLALTIFVGFTIPVNYMLGWSRW | 693 |
| ET299 | FYFLMNFLATLVMSAIFRCVGSATKTLSEAMVPASCLLLAISLYVGFSIPKKDLLGWSRW | 681 |
| ET293 | FYFLMNMVATFAMSHIFRCLGAATKTESEAMVPSSLLLLSMAIYTGFAIPKTKMLGWSKW | 658 |
| ET289 | FYFLMTMVATFLMSHIFRCLGASTKTYAESMVPASVLLLALSIYTGFAIPKTKILGWSKW | 680 |
| ET287 | FYFLMTMVATFLMSHIFRCLGASTKTYAESMVPASVLLLGLAIYTGFAIPKTKILGWSKW | 676 |
| ET291 | FYFLMTIVATFLMSHIFRCLGAATKTYAESMVSASLILLAQALYTGFAIPKTNILGWSKW | 704 |
| ET290 | FYFLMTMVGTFAMSHIFRCLGASTKTYAESMVPASLLLLALAIYTGFAIPKTKILGWAKW | 688 |
| ET282 | FYFLINIIAVFVMSHMYRCVGSLINTLTEAMVPASILLLAMAMYTGFAIPKTKMLGWSKW | 678 |
| PDR5 | FYLLINIVAVFSMSHLFRCVGSLIKTLSEAMVPASMLLLALSMYTGFAIPKKKILRWSKW | 696 |
| ET265 | FYWLINIVAVFAMSHLFRTVGSLTKTLSEAMIPASMLLLAMSMETGFAIPKTKMLGWSKW | 684 |
| | ** * .. ..: ** ::* :*: :.: :::: . :**. :::.**:** . :* *.:* | |
| ET306 | INYINPLAYAFEALMANEFHGLRYACSAFLPDNPDNHPDWPAKSWICNAVGAVAGEATVS | 744 |
| ET304 | INYLDPLAYAFEALMANEFAGVTYDCSSFVPGDPRSIPNIPSDGFICNAVGAQTGEFTVD | 753 |
| ET299 | IWYINPLSYIFESLMINEFVGRDFPCATFVPSGAGY-EDIGSLERVCNTVGAVPGNPRVS | 740 |
| ET293 | ISYIDPLSYIFESLMVNEFHGRKFQCSVYVPTGPAY-ANATGTERVCSAVGAVPGQDYVL | 717 |
| ET289 | IWYINPLSYVFESLMVNEFHDRRFDCSAYIPTGPAY-ESISGTERVCSAVGAVPGQDYVN | 739 |
| ET287 | IWYINPLSYVFESLMVNEFHGRRFACYAYIPTGPGY-LDVTGTEHVCSAVGAVPGQNYVD | 735 |
| ET291 | IWYINPLSYIFESLMVNEFHGRNFSCSQYIPAGSGY-EILSGTERVCSAVGAVPGQDFVS | 763 |
| ET290 | IWYINPLSYIFESLMVNEFHGRQFKCSQFVPAGPGY-ENVSGTQRVCSAVGALPGEDYVS | 747 |
| ET282 | IWWINPLSYLFESLMGNEFHDQKFPCTTFVPRGGDY-DHVTGTERVCSVVGSKAGQDYVL | 737 |
| PDR5 | IWYINPLAYLFESLLINEFHGIKFPCAEYVPRGPAY-ANISSTESVCTVVGAVPGQDYVL | 755 |
| ET265 | IWYINPIAYLFESLMINEFHGRRFECAAFIPSGPAY-SNITATERVCAVSGSVAGQSYVL | 743 |
| | * :::*::* **:*: *** . : * ::* . . :* . *: *: * | |
| ET306 | GDAYLDAAYSYSNSHKWRNWAITFAFCIFFLATYMIFAEYNESAKQKGEILLFQRSTLKK | 804 |
| ET304 | GTTYLEVAYKYKNSHRWRNWGITLAFALFFLAIYLVESEYNESAMQKGEVLLFQRSTLRK | 813 |
| ET299 | GLAFIEQTYGYSASHRWRSLGIGIAFFIFFTAFYLLFCEFNESAVQKGEILLFPKSVLKS | 800 |
| ET293 | GDDYLRLSYNYLHKHKWRGFGIGMAYVVFFLGVYLAFTEFNESARQRGEVLVFTRESLKK | 777 |
| ET289 | GETYINVAYGYYHSHKWRGLGIGLAYAIVFLGVYLAVTEFNESAKQRGEILVFPQAIMRR | 799 |
| ET287 | GETYINVAYGYYHAHKWRGLGIGLAYAIFFLGVYLAVVEFNESAKQRGEILVFPHWAMAR | 795 |
| ET291 | GETYINVAYGYYHAHKWRGLGIGLAYAIVFLAVYLAVTEFNESAKQRGEILVFPQSVMRR | 823 |
| ET290 | GERYINVAYNYYHSHKWRGLGIGLAYAIFFLGVYLAVTELNESAKQRGEILVFPQAVMRR | 807 |
| ET282 | GDDYLKESYGYLIKHKWRGFGVGMAYLIFFFFLYLFLCEVNEGAKQKGEILVFPRSIVRK | 797 |
| PDR5 | GDDFIRGTYQYYHKDKWRGFGIGMAYVVFFFFVYLFLCEYNEGAKQKGEILVFPRSIVKR | 815 |
| ET265 | GDDYIRVSYDYLHKHKWRGFGIGMAYAIFFLFAYLVVCEYNEGAKQKGEMLVFPQSVLRK | 803 |
| | * :: :* * .:**. .: :*: :.* *: . * **.* *:**:*:* : : | |
| ET306 | LKKEHKAAKNDIEGGK------------------------LRDITEQDHDEESE------ | 834 |
| ET304 | LKKEKAASQNELESGN------------------------EKGVVPN--GEDVD------ | 841 |
| ET299 | KRRQLSKSKND----------------------IETADDPEGGVTDQKLLQDSLEESNVS | 838 |
| ET293 | MKRAKKLESARGD--------------------AENSAGMETGINEKKLLEESGESS--- | 814 |
| ET289 | MKKQRHRSEKNNGGGARGSETTGGGGGGGSAADLESSAG-VVPVNEKKLLEDSTDSA--- | 855 |
| ET287 | MKKQRRLRAAG---ADPADEEHGGASS---------------GTTEKKMLEDSAEDD--- | 834 |
| ET291 | MKKERKLRNSSDY----------------SGTDVENSAG-SAPLNEKKMLDESSVSA--- | 863 |
| ET290 | MKKQRKLRAGTNA----------------NGSDIENTAG-VATLNEKKMLEESSNSV--- | 847 |
| ET282 | MRKQNKLKEEDR-DP----------------EDVEKIAG-SSGSTDKMLLKDSSESI--- | 836 |
| PDR5 | MKKRGVLTEKNANDP----------------ENVGERS--DLSSDRKMLQESSEE-E--- | 853 |
| ET265 | LRKEGQLKKDS--------------------EDIENGS--NSSTTEKQLLEDSDE-G--- | 837 |
| | :: : .. | |
| ET306 | -------QHVDAIQAGKDIFHWRDVHYTVKIKSEYREILSGVDGWVKPGILTALMGASGA | 887 |
| ET304 | -------KDVDVIHAGTQTFHWRDVHYTVKIKKEDREILSGVDGWVKPGILTALMGASGA | 894 |
| ET299 | SSSEKSANANVGLSKSEAIFHWRNVCYDVQIKKETRRILSNVDGWVEPGTLTALMGSSGA | 898 |
| ET293 | TSS----FQDVKLSQTEAIFHWRNVCFDVKIKKEDRRILNNVDGWVKPGILTALMGSSGA | 870 |
| ET289 | NSTNSSMGE-AGLTQSEAIYHWRNVCFDVPIKKETRRILDHVDGWVKPGTLTALMGASGA | 914 |
| ET287 | DASAGASADAGLISSSNAIFHWRNVCFDVAIKKETRRILDHVDGWVKPGTLTALMGASGA | 894 |
| ET291 | GST-SSMGD-AKLSKSEAIYHWRNVCFEVNIKKETRRILNNVDGWVKPGILTALMGASGA | 921 |
| ET290 | ASS-SSMGD-VKLSSSEATFHWKNVCFEVPIKKETRRILDNVDGWVKPGTLTALMGASGA | 905 |
| ET282 | DEE----NEPSALGGSQAIFHWRNLCYEVQIKGDTRRILNNVDGWVKPGTLTALMGASGA | 892 |
| PDR5 | SDT----YGEIGLSKSEAIFHWRNLCYEVQIKAETRRILNNVDGWVKPGILTALMGASGA | 909 |
| ET265 | SSN----GDSTGLVKSEAIFHWRNLCYDVQIKDETRRILNNVDGWVKPGTLTALMGSSGA | 893 |
| | : :**::: : * ** : *.**. *****:*********:*** | |
| | Walker A | |
| ET306 | GKTTLLDVLANRVTMGIVIGNMFVNGRLRDSSFQRSTGYVQQQDLHLPTATVREALRFSA | 947 |
| ET304 | GKTTLLDVLANRVTMGVVTGDMFVNGHLRDNSFQRSTGYVQQQDLHLRTATVREALKFSA | 954 |
| ET299 | GKTTLLDCLASRVTMGVITGDMFVNGHLRDNSFPRSIGYCQQQDLHLATATVRESLRFSA | 958 |
| ET293 | GKTTLLDCLASRVTTGVVTGDMFVNGHLRDASFARSIGYCQQQDLHLQTATVRESLRFAA | 930 |
| ET289 | GKTTLLDCLASRVTTGVITGDMEINGFLRDSSFARSIGYCQQQDLHLETSTVRESLRFSA | 974 |
| ET287 | GKTTLLDCLASRVTTGVITGDMFINGFLRDASFARSIGYCQQQDLHLETATVRESLRFSA | 954 |
| ET291 | GKTTLLDCLASRVTTGTITGDMFINGFLRDASFARSIGYCQQQDLHLESATVRESLRFAA | 981 |
| ET290 | GKTTLLDCLASRVTTGTITGDMFINGFLRDASFARSIGYCQQQDLHLQTATVRESLRFSA | 965 |
| ET282 | GKTTLLDCLAERVTMGVITGDIFVNGKIRDESFPRSIGYCQQQDLHLKTATVRESLIFSA | 952 |
| PDR5 | GKTTLLDCLAERVTMGVITGDILVNGIPRDKSFPRSIGYCQQQDLHLKTATVRESLRFSA | 969 |
| ET265 | GKTTLLDCLAERVTMGVITGDVLVDGRPRDESFPRSIGYCQQQDLHLKTSTVRESLRFSA | 953 |
| | ******* **.*** * :**:::::* ** ** ** ** ******* ::****:* *:* | |
| ET306 | YLRQPAEVSKAEKDDYVEEVIKILDMQKYADAVVGVAGEGLNVEQRKRLTIGVELAAKPK | 1007 |
| ET304 | YLRQPASVSTAEKDQYVEEVISILDMEKYADAVVGVAGEGLNVEQRKRLTIGVELAAKPK | 1014 |
| ET299 | YLRQSSEVSIEEKNSYVEDVLRILEMEPYADAVVGIAGEGLNVEQRKRLTIGVELAAKPK | 1018 |
| ET293 | YLRQPASVSTEEKNDYVEEIIRILDMEKYADAVVGVAGEGLNVEQRKRLTIGVELSAKPK | 990 |
| ET289 | YLRQPSHISKQEKDRYVEEVIRILEMLPYADAVVGVAGEGLNVEQRKRLTIGVELAAKPK | 1034 |
| ET287 | YLRQPDHVSIQDKNSYVDDVIRILEMEKYADAVVGVAGEGLNVEQRKRLTIGVELAAKPK | 1014 |
| ET291 | YLRQPATVSEDEKNKYVEDVIKILEMETYANAVVGVAGEGLNVEQRKRLTIGVELAAKPK | 1041 |
| ET290 | YLRQPSSVSKSEKEKYVEDVIKILEMETYADAVVGVAGEGLNVEQRKRLTIGVELAAKPK | 1025 |
| ET282 | MLRQPKSVPVSEKKKYVDDVIRILEMEQYADAVVGVPGEGLNVEQRKRLTIGVELVAKPK | 1012 |
| PDR5 | YLRQPAEVSIEEKNRYVEEVIKILEMEKYADAVVGVAGEGLNVEQRKRLTIGVELTAKPK | 1029 |
| ET265 | YLRQPAEVSVEEKDAYVEEVIKILEMEKYADAVVGVAGEGLNVEQRKRLTIGVELAAKPK | 1013 |
| | *** : :*. **:::: **:* **:****: ****************** **** | |
| | S-region | |
| ET306 | LLLFFDEPTSGLDSQTAWSICQLMRKLANHGQAILCTIHQPSAILMQEFDRLLFLAKGGR | 1067 |
| ET304 | LLLFLDEPTSGLDSQTAWSICQLMRKLANHGQAILCTIHQPSAILMQEFDRLLFLARGGK | 1074 |
| ET299 | LLLFLDEPTSGLDSQTAWSICQLMRKLADHGQAILCTIHQPSALLMQEFDRLLFLQKGGK | 1078 |
| ET293 | LLLFLDEPTSGLDSQTAWAICQLMRKLANHGQAILCTIHQPSALLMQEFDRLLFLKRGGR | 1050 |
| ET289 | LLLFLDEPTSGLDSQTAWSICQLMRKLANHGQAILCTIHQPSALLMQEFDRLLFLQRGGK | 1094 |
| ET287 | LLLFLDEPTSGLDSQTAWSICQLMRKLANHGQAILCTIHQPSALLMQEFDRLLELQRGGR | 1074 |
| ET291 | LLLFLDEPTSGLDSQTAWSICQLMRKLANHGQAILCTIHQPSALLMQEFDRLLFLQRGGQ | 1101 |
| ET290 | LLLFLDEPTSGLDSQTAWSICQLMRKLANHGQAILCTIHQPSALLMQEFDRLLFLQRGGK | 1085 |
| ET282 | LLVFLDEPTSGLDSQTAWSICQLMKKLSKHGQAILCTIHQPSAMLMQEFDRLLFLQKGGN | 1072 |
| PDR5 | LLVFLDEPTSGLDSQTAWSICQLMKKLANHGQAILCTIHQPSAILMQEFDRLLFMQRGGK | 1089 |
| ET265 | LLVFLDEPTSGLDSQTAWSICQLMRKLASHGQAILCTIHQPSAILMQEFDRLLFLQKGGK | 1073 |
| | **:*:*************:*****:**:.**************:**********: :**. | |
| | Walker B | |
| ET306 | TVYFGDLGEGCQTLIDYFEKYGAPKCPPEANPAEWMLHVIGAAPGSHANQDYHQVWLDSA | 1127 |
| ET304 | TVYFGDLGKNCQTLIDYFEKYGAPKCPPEANPAEWMLHVIGAAPGSHANQDYYQVWLNST | 1134 |
| ET299 | TIYFGDLGPGCETMIDYFESHGADKCPEGANPAEWMLEVIGAAPGSHANQDYHEVWRNSE | 1138 |
| ET293 | TVYFGDLGDGCSKMIDYFESQGAPKCPPGANPAEWMLEVIGAAPGSHTDKDYGEVWRNSD | 1110 |
| ET289 | TVYFGDLGPGCQTMIDYFESHGAHKCPDGANPAEWMLEVIGAAPGTHASQNYHDVWRNSD | 1154 |
| ET287 | TVYFGELGAGCQTMIDYFESHGSHKCPSGANPAEWMLEVIGAAPGTHAAQDYNEVWRNSD | 1134 |
| ET291 | TVYFGELGKGCHKMIDYFESNGAPRCPDGANPAEWMLAVIGAAPGTHANQDYHEVWRNSP | 1161 |
| ET290 | TVYFGDLGKGCQTMIDYFEKNGAHPCPAGANPAEWMLEVIGAAPGSHANQNYSEVWRNSS | 1145 |
| ET282 | TVYFGDLGKDCKTMIDYFESNGADPCPPDANPAEWMLEVVGAAPGTHANRDYHEAWRNSP | 1132 |
| PDR5 | TVYFGDLGEGCKTMIDYFESHGAHKCPADANPAEWMLEVVGAAPGSHANQDYYEVWRNSE | 1149 |
| ET265 | TVYFGELGEGCQVMIDYFERNGSHKCPPDANPAEWMLEVVGAAPGSHANQDYHEVWRNSE | 1133 |
| | *:***:** .* :***** *: ** ******** *:*****:*: ::* :.* :* | |
| ET306 | ERRDVLSELDRMEKELVNIP-VDDSVSHSEFAAPFWVQLTVVTARVFQQFWRTPSYIWAK | 1186 |
| ET304 | ERQEVKQELDRMERELSQLP-RDDSIDHNEYAAPEWKQYGIVTQRVFQQYWRSPIYIYSK | 1193 |
| ET299 | EYNAVQQKLDWMEVELAKKP-LDNSSEQSEEGTSIFYQCKIVTLRLFQQYYRTPSYIWSK | 1197 |
| ET293 | EYRAVQEELDWMERELPKRP-LDTAAEQTEFATSLETQYKLVTQRLFQQYWRTPSYLWSK | 1169 |
| ET289 | EYRAVQEELEWMERELPKKP-IDTSNEQSEESTSLFYQYCLVTKRLLEQYWRTPSYLWSK | 1213 |
| ET287 | EYRAVQEELEWMERELPNRPAVDTSSEQSEFSTSLVYQYSLVTHRLLQQYWRTPSYLWSK | 1194 |
| ET291 | EYRAVQEELEWMEQELPKKP-IDTSNEQTEFAASLLYQYYLVTKRLAEQYWRTPSYLWSK | 1220 |
| ET290 | EYKAVQEELEWMEKELPKRP-EDNSSEQTEFATSLVYQYTLVTKRLVEQYWRTPSYLWSK | 1204 |
| ET282 | EYQAVQQELDRLENELQSLDEEDGVEKHKSEATDVFTQIKFVSFRLQQQYWRSPQYLWSK | 1192 |
| PDR5 | EYRAVQSELDWMERELPKKGSITAAEDKHEFSQSIIYQTKLVSIRLFQQYWRSPDYLWSK | 1209 |
| ET265 | EFRIVHEELDLMERELPAKSAG-VDTDHQEFATGLFYQTKLVSVRLFQQYWRSPEYLWAK | 1192 |
| | * . * .:*: :* ** .: .:. . * .*: *: :*::*:* *:::* | |
| ET306 | MFLSVVSSLFIGFIFFRSKNSIQGLQNQMFALFMFLTIFNPLLQQILPTFVSQRDLYETR | 1246 |
| ET304 | LFLAISSSMFIGFAFFKAKNTRQGLQNQMFALFMFLVIFNALIQQTLPEYVRQRELYEVR | 1253 |
| ET299 | LFLTIFSQLFIGFTFFKAKLDMQGLQNQLEAVFTFTVIFNPACQQYLPLFVSQRDLYEAR | 1257 |
| ET293 | IILTLISQIFIGFTFFKSDSTLQGLQNQMLSIFMFTVVFNPTLQQYLPSFVSQRDLYEAR | 1229 |
| ET289 | IGLTIISQIFIGFTFFKADSSMQGLQNQMLSIFMFTVIFNPTLQQYLPTFVSQRDLYEAR | 1273 |
| ET287 | VGLTIISQLFIGFTFFKSDSSMQGLQNQMLSIFMFAVIFNPTLQQYLPTFVSQRDLYEAR | 1254 |
| ET291 | LILSVISQIFIGFTFFKADSSLQGLQNQMLSIFMFTLVFNPTLQQYLPTFVSQRGLYEAR | 1280 |
| ET290 | IGLTIISQIFIGFTFFKADNSMQGLQNQMLSIFMFSVIFNPTLQQYLPTFVAQRDLYEAR | 1264 |
| ET282 | FFLTVISELFIGFTFFKADRSMQGLQNQMLAVFMFTVIFNAILEQYLPNYVEQRDLYEAR | 1252 |
| PDR5 | FILTIFNQLFIGFTFFKAGTSLQGLQNQMLAVFMFTVIFNPILQQYLPSFVQQRDLYEAR | 1269 |
| ET265 | FVLTIFNELFIGFTFFKAGTSLQGLQNQMLAAFMFTVIFNPLLQQYLPSFVQQRDLYEAR | 1252 |
| | . *:: ..:**** **:: ******::: * * :** :* ** :* ** ***.* | |
| ET306 | ERPAKTFSWKAFIIAQFIAEAPWNAFVGTVGFFCFYYPAGFYRNAEPY--DEVNGRGAYG | 1304 |
| ET304 | ERPSKTFSWKAFITAQITSEVPWNALVGTIAFLVFYYPVGFYNNAAPNGSAEVHDRGAYA | 1313 |
| ET299 | ERPSRTFSWLAFIYSQIVVEIPFNVVIGTIGFFVFYYPIGFYNNASYS--DQLNERGVLF | 1315 |
| ET293 | ERPSRTFSWKAFILSQITVEAPWNEAVGTLGFLIYYYPVSFYRNASYA--HQLHERGALF | 1287 |
| ET289 | ERPSRTFSWLAFILSQISVEIPWNILAGTVGFFVYYFPVGFYNNASFA--DQLHERGALF | 1331 |
| ET287 | ERPSRIFSWVAFILSQITVEIPWNIFAGTIGFLVYYYPVGFYSNASYA--GQLHERGALF | 1312 |
| ET291 | ERPSKTFSWVSEMLSQITVEIPWNILAGTIGFIIYYYPVGFYNNASKA--GQLHERGALF | 1338 |
| ET290 | ERPSRIFSWVAFILSQITVEIPWNIIAGTIGFLVYYYPVGLYSTASKA--GQLHERGALF | 1322 |
| ET282 | ERPSRTFSWFAFIVSQILVEAPWNFLAGTIAYFIYYYPIGFYQNASAA--GQLHERGALF | 1310 |
| PDR5 | ERPSRTFSWISFIFAQIFVEVPWNILAGTIAYFIYYYPIGFYSNASAA--GQLHERGALF | 1327 |
| ET265 | ERPSRTFSWKAFIVSQILVEAPWNFLAGTLAYFIYYYPIGFYENASYA--GQLHERGALF | 1310 |
| | ***::**** :*: :*: * *:* **:.:: :*:* .:* .* ::: **. | |
| ET306 | WFFAVLFFIYIGSMAHMLIAPLQIADSAGNLGSLLFTMCLTFCGVLVTKDALPGFWVFMY | 1364 |
| ET304 | WFLTVLFFVYTGSFAHLVIAPLELADAAGNLASLIFTLCLTFCGVLVTSEGLPGFWIFMY | 1373 |
| ET299 | WLFSVAFYVFISSMGQLCIAGLEYAEAAGNLASLCFTMSLNFCGVEGGPGVLPGFWVFMY | 1375 |
| ET293 | WLFCTAFYVYVGSMGQLCIAGIEVAESAGHIASLMFTLSLSFCGVMVTPQNMPGFWKFMY | 1347 |
| ET289 | WLYATAFYVFTGSMAQLVIASQEVAQSAGQISSLLFTMCLSFCGVMVQPNNMPRFWIFMY | 1391 |
| ET287 | WLYATAFYVETGSMAQLVVAGQEVAQAAGQIASLLFTLSLSFCGVMVQPYNMPGFWIFMY | 1372 |
| ET291 | WLYCTAFYVETGSMAQVCIAGLDVAEAAGELGSLLYTLALSFCGVMVTPSNMPRFWLFMY | 1398 |
| ET290 | WLYATAYYVFTGSMAQLCIAGQEVAEPAGQMASLLFTLSLSFCGVLVGPSSMPGFWKFMY | 1382 |
| ET282 | FLWSTAFYVWVGSMALLANSFIEHDVSAANLANLCFTLALSFCGVMTTPDAMPHFWIFMY | 1370 |
| PDR5 | WLFSCAFYVYVGSMGLIVISFNQVAESAANLASLLFTMSLSFCGVMITPSAMPRFWIFMY | 1387 |
| ET265 | WLFSTAFYVYVGSMGELTVSFNEIAENAANLASLMFTMALSFCGVMTTPSAMPRFWIFMY | 1370 |
| | :: :::: .*:. : : : *..:..* :*:.*.****: :* ** *** | |
| ET306 | RVSPETYFIEGYLINALAHNKIVCSEEEFRVLSPPDGLTCQDYLGDYISKAGTGYLQDPE | 1424 |
| ET304 | RVSPETYFIDGELSNAVAHNVVKCSDSELVHFSPPQGATCGDYMKEYLEKAGTGYVEDPS | 1433 |
| ET299 | RVSPLSYFIDGVLSTALANNPVTCADYEYLSFVPKSGETCGEYMSTYIATYG-GYILDPD | 1434 |
| ET293 | RASPLTYFIDGLMSTGLANAPAHCSHYELVSFTPPAGQTCGEYMAPYIKMAGTGYLTSPS | 1407 |
| ET289 | RVSPLTYFIDGTLSTGLANADVHCSDYEMVSFTPPQGQTCGQYMQSYISSAGTGYLADPD | 1451 |
| ET287 | RVSPLTYFIDGALSTGIANAKVHCADYEMVRFTPPQGQTCGQYMQPYITAAGTGYLKDSG | 1432 |
| ET291 | RVSPITYFIDGVLSTGVANADVHCADYEMVRFTPPAGQTCGQYMSRYIETTGTGYLDDPS | 1458 |
| ET290 | RVSPLTYFVDGTLSTGIANGKVQCSDYELVKFKPANGMTCGEYMEPYIKLVGTGYLSDPS | 1442 |
| ET282 | RVSPLTYFIDAVLAVGIANVDIECSNYEFVQFTPPQGRTCGEYMQAYLKSAGTGYLKDAN | 1430 |
| PDR5 | RVSPLTYFIQALLAVGVANVDVKCADYELLEFTPPSGMTCGQYMEPYLQLAKTGYLTDEN | 1447 |
| ET265 | RVSPLTYFVQGILAVGLANTKIECSSSEFLQFEAPSGMTCGNYMEAYLDYAGTGYLKDES | 1430 |
| | *.**::**::. :: .:*: *: * : * ** :*: *: **: . | |
| ET306 | ATGSCQFCPMSKTDDFLAQVQLDYGNRWRDVGIFIAFIFINLFFAVLFYWLARVPKKSDR | 1484 |
| ET304 | STSECGFCSISSTDAFLKVVKLDYGRRWRNVGIFIAFIVINWILAVFFYWLARVPKKNDR | 1493 |
| ET299 | ATDECSFCRISSTNAFLSSFQSSYHRRWRNEGIFIVFIVENWAGCIFFYWLARVPKKNNR | 1494 |
| ET293 | ATDKCSFCPVSTTNDYLAQVSSHYKDRWRNWGIFICYIFIDFGFAIFFYWLARVPKKKNR | 1467 |
| ET289 | ATDECRFCSVSSTNDYLKAVSSEYTHRWRNYGIFLAFIMFNFAAAVFFYWLARVPKKRNR | 1511 |
| ET287 | ATDECQFCSVSTTNDYLKAVSSSYSHRWRNYGIFLAFIMFNFAAAVFFYWLARVPKKRNR | 1492 |
| ET291 | AMDECKFCSVSDTNEFLKAVISSYDHRWRNYGIFLVFIFVNFALASFLYWLMRVPKKRNR | 1518 |
| ET290 | ATDECRFCSVSTTNDYLSAVSSSYSRRWRNYGIFLVYIFFNFAMAVFIYWLARVPKKRNR | 1502 |
| ET282 | ATAQCLLCPLSRTNDYLSQVNSHYSHRWRNYGIFICYIVFNYVAAVFLYWLARVPKKDTF | 1490 |
| PDR5 | ATDTCSFCQISTTNDYLANVNSFYSERWRNYGIFICYIAFNYIAGVFFYWLARVPKKNGK | 1507 |
| ET265 | ATGTCEFCEYSYTNDYLSSINSYYSQRWRNWGIFICYIAINYIGGIAFYYLARVPKKSKV | 1490 |
| | : * :* * *: :* . * ***: ***: :* .: :*:* ***** | |
| ET306 | VSTEQPEGAVNMGAELEKKAALHRT-ATNAASQAASQGYAPQVYNEKVGSEEGSLDKVDN | 1543 |
| ET304 | VKSGAEAESTNKTVQKQETASIIEKEESSSNSKA-------------------------- | 1527 |
| ET299 | VANERNPDRETTKQISTHGE-------KS-KPQQIE-----QV----------------- | 1524 |
| ET293 | VADERDPDAPKKSVAGTKN----------------------------------------- | 1486 |
| ET289 | VADERKPEAQKIQEK--------------------------------------------- | 1526 |
| ET287 | VADERKSDAQKLKEK--------------------------------------------- | 1507 |
| ET291 | VVDERKPEAQKLASK--------------------------------------------- | 1533 |
| ET290 | VADERKTEAQKLVEK--------------------------------------------- | 1517 |
| ET282 | FSKIESKK---------------------------------------------------- | 1498 |
| PDR5 | LSKK-------------------------------------------------------- | 1511 |
| ET265 | AKK--------------------------------------------------------- | 1493 |
| ET306 | SDSSR | 1548 |
| ET304 | ----- | 1527 |
| ET299 | ----- | 1524 |
| ET293 | ----- | 1486 |
| ET289 | ----- | 1526 |
| ET287 | ----- | 1507 |
| ET291 | ----- | 1533 |
| ET290 | ----- | 1517 |
| ET282 | ----- | 1498 |
| PDR5 | ----- | 1511 |
| ET265 | ----- | 1493 |

As can be seen by the consensus shown with asterisks, there is a high degree of conservation in certain regions of the PDR5 homologs that show activity for exporting nororipavine products. Studies of the Saccharomyces cerevisiae PDR5 have predicted the structure of the transmembrane protein and identified areas important for activity and binding (E. Balzi et al, JBC Vol. 269, pp 2206-2214, 1994). These residues are shown in bold in Table 10. The consensus region of PDR5 and its homologs are predicted as follows: Walker A sequences corresponding to amino acid residues of S. cerevisiae PDR5 are 193-200 GRPGSGC(S/T), and residues 905-912 G(A/S)SGAGKT. The Walker B conserved regions are (F/L)QCWD at residues 329-333 of S. cerevisiae PDR5 and 1030-1035 LL(V/L)F(L/F)D. The ABC transporter signature(S) in PDR5 and its homologs is VSGGERKRVSIA at 309-320 and LNVEQRKRLTIG at residues 1010-1021.

### Example 9 Effect of ABC Transporters on Oripavine Production from Thebaine

The effect of a subset of the ABC transporters from previous examples was tested in bioconversion reactions where an O-demethylase was used to convert thebaine to oripavine.

sOD1133 is a Saccharomyces cerevisiae yeast strain comprising recombinant polynucleotide sequences expressing a P450 O-demethylase (modified version of SEQ ID NO: 236), CPR (SEQ ID NO: 305, encoded by SEQ ID NO: 306) and an uptake transporter (SEQ ID NO: 623, encoded by SEQ ID NO: 624), which enable it to import and convert thebaine to oripavine. The background of S. cerevisiae sOD1133 is similar to the commonly available strain S288C (genotype MATa his340 leu2A0 ura3A0) (see the Saccharomyces Genome Database (SGD)). Different putative efflux transporters were overexpressed in sOD1133 using plasmid RPB15. An empty RPB15 control plasmid is the negative control for the data in this example. RPB15 is a derivative of vector p416TEF (Mumberg, 1995). One of skill in the art will know that other uptake transporters, O-demethylases, or CPRs may be used to achieve similar results. For example, T193_AanPUP3_55 (SEQ ID NO: 613), T198_AcoT97_GA (SEQ ID NO: 623), T149_AcoPUP3_59 (SEQ ID NO: 537) and/or T122_PsoPUP3_17 (SEQ ID NO: 487) have shown particularly effective for thebaine uptake. Further suitable transporter proteins are disclosed in WO2020/078837. Suitable demethylase enzymes (and accompanying CPRs) for conversion of thebaine to oripavine or northebaine to nororipavine include but are not limited to: SEQ ID Nos. 222, 224 and 236 when individually expressed in a yeast strain that contains demethylase-CPR Ce_CPR as described in WO 2021/069714 A1, and SEQ ID Nos. 198 and 874 or variants thereof, or additional enzymes that have both N- and O-demethylase activity such as those described in paragraphs 0124-0127 of WO 2021/069714 A1.

All experiments were run in triplicates. At first the strains were grown aerobically as 96-deep well cultures at 30° C. for 24 h in Delft media pH 5.5. This pre-culturing was followed by 10× dilution of the cultures with fresh Delft media pH 5.5 including the addition of 1 mM thebaine. Again, strains were grown aerobically as a 96-deep well culture at 30° C. After 72 hours total broth samples were prepared. For the total broth samples an aliquot of the total cell culture was taken and mixed 1:1 with 0.2% formic acid in water and heated to 80° C. for 10 minutes. After heating cells were pelleted by centrifugation and supernatants were diluted 1:1 in 0.1% formic acid in water and analyzed. Opioids were quantified as described in Example 1. Analysis of thebaine/oripavine samples was performed as described in Example 1, with separation achieved on a Kinetex F5 column (100×2.1 mm, 1.7 μm, 100 Å, Phenomenex, Torrance, CA, USA) using 0.05% (v/v) formic acid in H₂O and 0.05% (v/v) formic acid in acetonitrile as mobile phases A and B, respectively using the time-gradient as shown in Table 11.

TABLE 11

| Time [min] | B % |
|---|---|
| 0.0-1.0 | 2% |
| 1.0-5.2 | 2-33% |
| 5.2-5.5 | 33-100% |
| 5.5-6.5 | 100% |
| 6.5-6.7 | 100-2% |
| 6.7-7 | 2% |

1.5 min postrun

The injection volume was 1 μL and the flow rate was 600 L/min. The column temperature was maintained at 30° C. The liquid chromatography system was coupled to an Agilent 1290 diode array detector (Agilent Technologies, Palo Alto, CA, USA). UV-spectra were acquired at 220, 254 and 285 nm.

Co-expression of efflux transporters ET72 and ET319 increased the total conversion of thebaine to oripavine in sOD1133 as compared to no expression of heterologous exporters. Without being bound by theory, these transporters transport oripavine out of the cells, thus resulting in higher oripavine production by creating a product sink of end material out of the yeast cytosol.

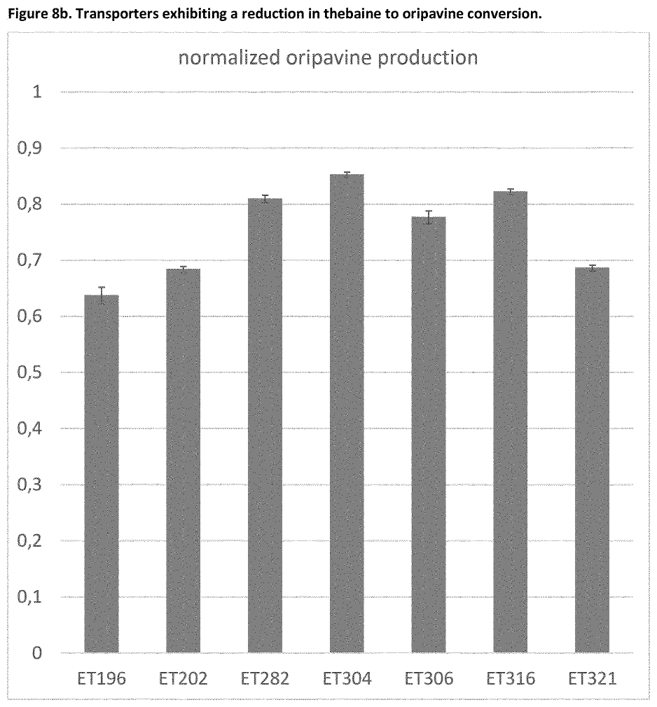

Several transporters had activity that was similar to the negative control (empty plasmid), meaning no activity for oripavine transport. However, transporters ET196, ET202, ET282, ET304, ET306, ET316 and ET321 actually reduced the production of oripavine as shown in FIG. 8b. These transporters must transport the substrate thebaine out of cells thus resulting in a lower yield due to less thebaine available for bioconversion as well as a futile cycle of import/export of thebaine.

While thebaine excretion is disadvantageous in the conversion of thebaine to oripavine, it is expected that these transporters would improve production of thebaine in strains where thebaine is the end product, i.e. in strains producing thebaine e.g. from glucose or tyrosine derivatives such as those described by Han, J., Wu, Y., Zhou, Y. et al. (Engineering Saccharomyces cerevisiae to produce plant benzylisoquinoline alkaloids. aBIOTECH 2, 264-275 (2021). https://doi.org/10.1007/s42994-021-00055-0). It has been shown previously that efflux of end product molecules from genetically engineered strains improves their production (Z. M. Belew, M. Poborsky, H. H. Nour-Eldin, B. A. Halkier, Transport engineering in microbial cell factories producing plant specialized metabolites, Current Opinion in Green and Sustainable Chemistry, https://doi.org/10.1016/j.cogsc.2021.100576).)

### REFERENCES

Bateman et al., Nucl. Acids Res., 27:260-262 (1999).
Bitter B G A, Egan K M, Koski R A, Jones M O, Elliott S G, Giffin J C. Expression and secretion vectors for yeast. Methods Enzymol. 153:516-44. pmid: 2828848 (1987)
Boswell-Casteel, Rebba C; Franklin A; Hays; Equilibrative Nucleoside Transporters-A Review; Nucleosides Nucleotides Nucleic Acids; 2017 Jan. 2; 36 (1): 7-30.
Brohée et al. (“YTPdb: A wiki database of yeast membrane transporters”; Biochimica et Biophysica Acta 1798 (2010) 1908-1912)
Carroll, R. J. et al, J. Org. Chem. 2009, 74, 2, 747-752 Dec. 11, 2008;
Dastmalchi et al. (“Purine permease-type benzylisquinoline alkaloid transporters in opium poppy”; Plant Physiology Preview. DOI: 10.1104/pp. 19.00565 (2019))
Dasgupta, A., Chapter 2—Prescription Opioids: An Overview, Editor(s): Amitava Dasgupta, Fighting the Opioid Epidemic, Elsevier, 2020, Pages 17-41
Dias P J, et al. (2010) Evolution of the 12-spanner drug: H+ antiporter DHA1 family in hemi-ascomycetous yeasts. OMICS 14 (6): 701-10
Fossati, E. et al. Synthesis of Morphinan Alkaloids in Saccharomyces cerevisiae. PLOS One. 2015 Apr. 23; 10 (4): e0124459.→Codeine and morphine from thebaine
Galanie et al. (“Complete biosynthesis of opioids in yeast”; Science. 2015 Sep. 4; 349 (6252): 1095-1100)
Hansen B. G. et al (2011); Versatile enzyme expression and characterization system for Aspergillus nidulans, with the Penicillium brevicompactum polyketide synthase gene from the mycophenolic acid gene cluster as a test case; App. and Environmental Microbiology; 77 (9): 3044-3051.
Hudlicky, T., 2015; Recent advances in process development for opiate-derived pharmaceutical agents; Canadian Journal of Chemistry. 93 (5): 492-501.
Jørgensen et al. (“Origin and evolution of transporter substrate specificity within the NPF family”; eLife 2017; 6: e19466. DOI: 10.7554/eLife.19466)
Jørgensen et al. (“A Functional EXXEK Motif is Essential for Proton Coupling and Active Glu-cosinolate Transport by NPF2.11”; Plant Cell Physiol. 56 (12): 2340-2350 (2015))
Katzmann, D J, T C Hallstrom, M Voet, W. Wysock, J. Golin, J. Volckaert, and W. S. Moye-Rowley. 1995. Expression of an ATP-Binding Cassette Transporter-Encoding Gene (YOR1) Is Required for Oligomycin Resistance in Saccharomyces cerevisiae. MOLECULAR AND CELLULAR BIOLOGY, December: 6875-6883
Kawahara T, et al.; 1997; Endoplasmic reticulum stress-induced mRNA splicing permits synthesis of transcription factor Hac1p/Ern4p that activates the unfolded protein response; Molecular Biology Cell; 8 (10): 1845-1862
Krainer, F. W., et al.; 2015; Optimizing cofactor availability for the production of recombinant heme peroxidase in Pichia pastoris; Microbial cell factories; 14, 4.
Krishnamurthy P, Xie T, Schuetz J D.; 2007; The role of transporters in cellular heme and porphyrin homeostasis; Pharmacol Ther.; 114, 345-358.
Maury J, et al. EasyCloneMulti: A Set of Vectors for Simultaneous and Multiple Genomic Integrations in Saccharomyces cerevisiae. PLOS One 11 (3): e0150394 (2016)
Michener, J. K., et al.; 2012; Identification and treatment of heme depletion attributed to overexpression of a lineage of evolved P450 monooxygenases. Proceedings of the National Academy of Sciences. 109; 19504-19509.
Mikkelsen et al.; Microbial production of indolylglucosinolate through engineering of a multi-gene pathway in a versatile yeast expression platform; Metabolic Engineering; Volume 14; Issue 2; Pages 104-111; March 2012.
Mumberg D, Müller R, Funk M.; Yeast Vectors for the Controlled Expression of Heterologous Proteins in Different Genetic Backgrounds; Gene; 156; 1995.
Narcross L, Fossati E, Bourgeois L, Dueber J E, Martin V J J. Microbial Factories for the Production of Benzylisoquinoline Alkaloids. Trends Biotechnol. 2016 March; 34 (3): 228-241.
Nielsen, J. B., M. L. Nielsen, and U. H. Mortensen; (2008); Transient disruption of nonhomologous end-joining facilitates targeted genome manipulation in the filamentous fungus Aspergillus nidulans; Fungal Genet. Biol.; 45:165-170.
Nour-Eldin H. H., Hansen B. G., Nørholm M. H., Jensen J. K., Halkier B. A. (2006); Advancing uracil-excision based cloning towards an ideal technique for cloning PCR fragments; Nucleic Acids Res; 34: e122.
Osmani et al., 2009, Phytochemistry 70:325-347
Protchenko, O., and C. C. Philpott; 2003; Regulation of intracellular heme levels by HMX1, a homologue of heme oxygenase, in Saccharomyces cerevisiae; J. Biol. Chem.; 278:36582-36587.
Pyne M E, Kevvai K, Grewal P S, Narcross L, Choi B, Bourgeois L, Dueber J E, & Martin V J J; A yeast platform for high-level synthesis of natural and unnatural tetrahydroisoquinoline alkaloids; BioRxiv preprint doi: https://doi.org/10.1101/863506, posted Dec. 5, 2019.
Ramanathan, V. S. and Chandra, P; Bulletin on Narcotics-1980, Issue 2.
Santella, M., 2021; Preparation Of Buprenorphine; WO/2021/144362
Sipos et al. (“First Synthesis and Utilization of Oripavidine—a concise and Efficient Route to Important Morphinans and Apomorphines”; Helvetica Chimica Acta. Vol. 92:1359-1365 (2009))
Sonnhammer et al., Nucl. Acids Res., 26:320-322 (1998).
Sonnhammer et al., Proteins, 28:405-420 (1997).
Stincone A, et al.; 2015; The return of metabolism: biochemistry and physiology of the pentose phosphate pathway; Biol Rev Camb Philos Soc.; August; 90 (3): 927-963.
Thorton, John D; Science and practice of liquid-liquid extraction Oxford: Clarendon Press, 1992-Chapter by Michael S. Verrall
Tolkachev, O. N., Shemeryankin, B. V. & Pronina, N. V. Isolation and purification of alkaloids. Chem Nat Compd 19, 387-400 (1983).
Van Wiltenburg, J., Santella, M., Roussel, P.; 2018; Preparation Of Buprenorphine; WO/2018/211331
Walker, J. E., M. Saraste, M. J. Runswick, and N. Gay. 1982. Distantly related sequences in the alpha and beta subunits of ATP synthase, myosin, kinases and other ATP-requiring enzymes and a common nucleotide binding fold. EMBO J. 8:945-951.
Yu X W, et al.; 2017; Identification of novel factors enhancing recombinant protein production in multi-copy Komagataella phaffii based on transcriptomic analysis of overexpression effects; Sci. Rep. November; 24; 7 (1): 16249

### ITEMS OF THE INVENTION

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention. In some aspects, the present invention may be presented in the following itemized embodiments.

Item 1. A recombinant microbial host cell capable of producing one or more BIA, BIA-glycoside, oripavine or glycosylated oripavine or glucosylated oripavine, thebaine, northebaine, nororipavine or glycosylated nororipavine or glucosylated nororipavine, wherein the host cell comprises a recombinant polynucleotide comprising a promoter operably linked to an ABC transporter effluxing one or more BIA or BIA-glycoside products.

Item 2. The recombinant microbial host cell of item 1, wherein one or more of the ABC transporters are one or more selected from:
a. present in the host cell at a gene copy number greater than in the wild type of the microbial host cell,
b. operably linked to a constitutive promoter or an inducible promoter that induces expression during cell exponential phase and BIA production, or
c. are under regulatory control such that a higher level of gene expression is induced by growth medium, growth conditions, the presence of an activator or transcription factor or absence of a repressor for an inducible promoter governing expression of the one or more endogenous ABC transporters, or any combination thereof.

Item 3. The recombinant microbial host cell of item 1 or 2, wherein the increased expression of one or more endogenous ABC transporters results from elevated levels of one or more transcription factors, wherein the one or more transcription factors are selected from:
a. polypeptide sequence having at least 75%, such as at least 80%, such as at least 85%, such as at least 90%, such as at least 95%, such as at least 99%, or 100% identity to SEQ ID No. 902, 904, 906, 908, or
b. encoded by a nucleic acid sequence having at least 75%, such as at least 80%, such as at least 85%, such as at least 90%, such as at least 95%, such as at least 99%, or 100% identity to SEQ ID No. 901, 903, 905, 907, or genomic DNA thereof.

Item 4. The recombinant microbial host cell of any preceding item, wherein the transcription factor is PDR 1, PDR3, PDR8 and/or YRR1.

Item 5. A recombinant microbial host cell according to any preceding item, wherein the recombinant microbial host cell excretes the BIA, BIA-glycoside, oripavine, glycosylated oripavine or glucosylated oripavine, thebaine, northebaine, nororipavine, glycosylated nororipavine or glucosylated nororipavine produced by the recombinant microbial host cell, at greater than 2%, preferably greater than 5%, preferably greater than 10%, preferably greater than 20% more excretion compared to a negative control recombinant microbial host cell not expressing the ABC transporter during cell exponential phase and BIA production.

Item 6. A recombinant microbial host cell according to any preceding item, wherein the recombinant microbial host cell produces the one or more of the BIAs at greater than 2%, preferably more than 5%, preferably more than 10%, preferably more than 20%, preferably more than 50% more than a negative control recombinant microbial host cell not expressing the ABC transporter during cell exponential phase and BIA production comprising no heterologous ABC transporter effluxing the one or more BIA or BIA-glycoside products.

Item 7. A recombinant microbial host cell according to any preceding item, wherein the one or more excreted BIAs are thebaine, nororipavine, oripavine, glucosylated oripavine or glucosylated nororipavine.

Item 8. The recombinant microbial host cell of any preceding item, wherein the ABC transporter is an ABC transporter involved in drug efflux or xenobiotic efflux.

Item 9. The recombinant microbial host cell of any preceding item, wherein the ABC transporter comprises a Walker A sequence G(A/S/R)(S/T)GAGK(S/T), a linker sequence (L/V)SGG(E/Q), and a Walker B sequence comprising four hydrophobic residues, an optional additional fifth hydrophobic residue and a D such that (I/L)(I/L)(I/V/L)(F/L/M)XD where X represents the optional additional hydrophobic residue or no additional residue.

Item 10. The recombinant microbial host cell of any preceding item, wherein the ABC transporter is:
a. an ABCC/multi-drug resistance associated protein (MRP) ABC transporters, or
b. an ABCG/pleiotropic drug resistance (PDR) ABC transporters.

Item 11. The recombinant microbial host cell of any preceding item, wherein the ABC transporter is not native to a BIA-producing plant.

Item 12. The recombinant microbial host cell of any preceding item, wherein the ABCC/multi-drug resistance associated protein (MRP) ABC transporter is:
a. a polypeptide comprising a sequence having at least 45%, such as at least 60%, such as at least 70%, such as at least 80%, such as at least 85%, such as at least 90%, such as at least 95%, such as at least 99%, such as 100% identity to SEQ ID No. 872, 910, 912, 914, 918, 920, 922, 924, 926, 928, 930, 932, 934, 936, 938, 940, 942, 956, 960, 962, 964, 966, 970, 1032, 1034, 1038 or 1040 or
b. encoded by a nucleic acid sequence having at least 45%, such as at least 60%, such as at least 70%, such as at least 80%, such as at least 85%, such as at least 90%, such as at least 95%, such as at least 99%, such as 100% identity to SEQ ID No. 871, 909, 911, 913, 917, 919, 921, 923, 925, 927, 929, 931, 933, 935, 937, 939, 941, 955, 959, 961, 963, 965, 969, 1031, 1033, 1037 or 1039 or genomic DNA thereof.

Item 13. The recombinant microbial host cell of any of items 1 to 12, wherein the ABC transporter comprises Walker A sequences G(X)(I/V)G(S/T)GK where X is a residue selected from P, L, S, A, V or M and GRTGAGK, two linker sequences comprising LSGGQ and NFSLGE, and Walker B sequences (I/V/T)(I/Y/V)L(M/F/L)D and I(I/L)(I/V)(L/M)D.

Item 14. The recombinant microbial host cell of any of items 1 to 11, wherein the ABCG/pleiotropic drug resistance (PDR) ABC transporter is:
a. a polypeptide comprising a sequence having at least 45%, such as at least 60%, such as at least 75%, such as at least 80%, such as at least 85%, such as at least 90%, such as at least 95%, such as at least 99%, such as 100% identity to SEQ ID No. 916, 976, 980, 986, 988, 990, 994, 996, 1010, 1012, 1018, 1020, 1022, 1026, 1028 or 1030, or
b. encoded by a nucleic acid sequence having at least 70%, such as at least 80%, such as at least 85%, such as at least 90%, such as at least 95%, such as at least 99%, such as 100% identity to SEQ ID No. 915, 975, 979, 985, 987, 989, 993, 995, 1009, 1011, 1017, 1019, 1021, 1025, 1027 or 1029 or genomic DNA thereof.

Item 15. The recombinant microbial host cell of any of items 1 to 11 or item 14, wherein the ABC transporter comprises Walker A sequences GRPGSGC(S/T) and G(A/S)SGAGKT, linker sequences VSGGERKRVSIA and LNVEQRKRLTIG, and Walker B sequences (F/L)QCWD and LL(V/L)F(L/F)D.

Item 16. The recombinant microbial host cell of any preceding item, further comprising:
a) one or more heterologous CYP demethylases capable of converting thebaine into northebaine, thebaine into oripavine, northebaine into nororipavine and/or oripavine into nororipavine, and one or more demethylase cytochrome P450 reductase (demethylase-CPR), and/or
b) heterologous sequences encoding:
   i. a tyrosine hydroxylase (TH) converting L-tyrosine into L-dopa, and
   ii. optionally, a TH-CPR capable of reducing the TH of i), and
   iii. a L-dopa decarboxylase (DODC) converting L-dopa into dopamine, or a tyrosine decarboxylase (TYDC) converting L-dopa into dopamine, and
   iv. a monoamine oxidase converting dopamine into 3,4-DHPAA, or a N-methyl-coclaurine hydroxylase (NMCH) converting (S)-Coclaurine into (S)-3′-hydroxycoclaurine and/or (S)—N-Methylcoclaurine into (S)-3′-Hydroxy-N-Methylcoclaurine; and
   v. a norcoclaurine synthase (NCS) converting Dopamine and 4-HPAA into (S)-norcoclaurine and/or 3,4-DHPAA and dopamine to NLDS, and
   vi. a 6-O-methyltransferase (6-OMT) converting (S)-norcoclaurine into (S)-Coclaurine and/or norlaudanosoline into (S)-3′-Hydroxy-coclaurine, and
   vii. a coclaurine-N-methyltransferase (CNMT) converting (S)-Coclaurine into (S)—N-Methylcoclaurine and/or (S)-3′-hydroxycoclaurine into (S)-3′-hydroxy-N-methyl-coclaurine, and
   viii. a 3′-hydroxy-N-methyl-(S)-coclaurine 4′-O-methyltransferase (4′-OMT) converting (S)-3′-Hydroxy-N-Methylcoclaurine into (S)-reticuline, and
   ix. a 1,2-dehydroreticuline synthase-1,2-dehydroreticuline reductase (DRS-DRR) converting (S)-reticuline into (R)-reticuline comprised of one or more proteins, and
   x. a salutaridine synthase (SAS) converting (R)-reticuline into Salutaridine, and
   xi. a salutaridine reductase (SAR) converting Salutaridine to Salutaridinol, and
   xii. a salutaridinol 7-O-acetyltransferase (SAT) converting Salutaridinol into 7-O-acetylsalutaridinol, and
   xiii. a thebaine synthase (THS) converting 7-O-acetylsalutaridinol or 7-O-acetylsalutaridinol acetate into thebaine;
c) and optionally, one or more glycosyl transferases capable of transferring a glycosyl moiety to a BIA, oripavine or nororipavine.

Item 17. The recombinant microbial host cell of item 16, wherein the one or more demethylases is:
a. an N-demethylase comprising a polypeptide sequence having at least 75%, such as at least 85%, such as at least 90% or at least 95% identity to SEQ ID No. 140, 152, 198, 250, 252, 843, or
b. an N-demethylase encoded by a nucleic acid sequence having at least 75%, such as at least 85%, such as at least 90% or at least 95% identity to 141, 153, 199, 251, 253, 844, or genomic DNA thereof, or
c. an O-demethylase comprising a polypeptide sequence having at least 75%, such as at least 85%, such as at least 90% or at least 95% identity to SEQ ID No. 198, 222, 224, 236, or
d. an O-demethylase encoded by a nucleic acid sequence having at least 75%, such as at least 85%, such as at least 90% or at least 95% identity to SEQ ID No. 199, 223, 225, or 237, or genomic DNA thereof.

Item 18. The recombinant microbial host cell of item 16 or 17, wherein the one or more CPRs:
a. comprises a polypeptide sequence having 75%, such as at least 85%, such as at least 90% or at least 95% identity to SEQ ID No. 292 or 305, or
b. is encoded by a nucleic acid sequence having at least 75%, such as at least 85%, such as at least 90% or at least 95% identity to SEQ ID No. 293 or 306, or genomic DNA thereof.

Item 19. The recombinant microbial host cell of any preceding item, wherein the one or more glycosyltransferases (UGT) is an aglycone O-UGT or an aglycone O-glucosyltransferase.

Item 20. The recombinant microbial host cell of any preceding item, wherein the one or more glycosyltransferases (UGT):
a. comprises an amino acid sequence having at least 60%, such as at least 70%, such at least 75%, such as at least 80%, such as at least 90%, such as at least 95%, such as at least 99%, such as 100% identity to a UGT comprised in any one of SEQ ID NO: 880, 882, 878, 884, 886, 888, 890, 892, 894, 896, or 898; or
b. is encoded by a nucleic acid sequence having at least 60%, such as at least 70%, such at least 75%, such as at least 80%, such as at least 90%, such as at least 95%, such as at least 99%, such as 100% identity to SEQ ID No. 879, 881, 877, 883, 885, 887, 889, 891, 893, 895 or 897, or genomic DNA thereof.

Item 21. The recombinant microbial host cell of any preceding item, wherein the recombinant microbial host cell is a yeast.

Item 22. The recombinant microbial host cell of any preceding item, wherein the recombinant microbial host cell is Saccharomyces cerevisiae.

Item 23. The recombinant microbial host cell of any preceding item, further comprising an uptake transporter capable of transporting an opioid, such as oripavine, into the recombinant host cell.

Item 24. The recombinant microbial host cell of item 23, wherein the uptake transporter is a polypeptide:
a. comprising an amino acid sequence having at least 60%, such as at least 70%, such at least 75%, such as at least 80%, such as at least 90%, such as at least 95%, such as at least 99%, such as 100% identity to an uptake transporter comprised in any one of SEQ ID NO: 307, 311, 317, 461, 473, 733, or 735.
b. encoded by a nucleic acid sequence comprising at least 60%, such as at least 70%, such at least 75%, such as at least 80%, such as at least 90%, such as at least 95%, such as at least 99%, such as 100% identity to 308, 312, 318, 462, 474, 734, 736, or genomic DNA thereof.

Item 25. The recombinant microbial host cell of any preceding item, further comprising an operative biosynthetic pathway capable of producing the thebaine, northebaine, oripavine and/or nororipavine, wherein the pathway comprises one or more polypeptides selected from:
a) a 3-deoxy-D-arabino-2-heptulosonic acid 7-phosphate synthase (DAHP synthase) converting PEP and E4P into DAHP;
b) a 5-enolpyruvylshikimate-3-phosphate (EPSP) synthase (aro1) converting 3-phosphoshikimate and PEP into EPSP;
c) an aro1 polypeptide converting DHAP and PEP into EPSP;
d) a 276horismite synthase converting EPSP into Chorismate;
e) a 276horismite mutase converting Chorismate into prephenate;
f) a prephenate dehydrogenase (Tyr1) converting prephenate into 4-HPP;
g) an aromatic aminotransferase converting 4-HPP into L-Tyrosine;
h) a tyrosine hydroxylase (TH) converting L-tyrosine into L-dopa
i) a TH-CPR capable of reducing the TH of h);
j) a L-dopa decarboxylase (DODC) converting L-dopa into dopamine;
k) a Tyrosine decarboxylase (TYDC) converting L-dopa into dopamine;
l) a hydroxyphenylpyruvate decarboxylase (HPPDC) converting 4-HPP into 4-HPPA;
m) a monoamine oxidase converting dopamine into 3,4-DHPAA;
n) a norcoclaurine synthase (NCS) converting Dopamine and 4-HPAA into (S)-norcoclaurine;
o) a 6-O-methyltransferase (6-OMT) converting (S)-norcoclaurine into (S)-Coclaurine and/or norlaudanosoline into (S)-3′-Hydroxy-coclaurine;
p) a coclaurine-N-methyltransferase (CNMT) converting (S)-Coclaurine into (S)—N-Methylcoclaurine and/or (S)-3′-hydroxycoclaurine into (S)-3′-hydroxy-N-methyl-coclaurine;
q) a N-methyl-coclaurine hydroxylase (NMCH) converting (S)-Coclaurine into (S)-3′-hydroxycoclaurine and/or (S)—N-Methylcoclaurine into (S)-3′-Hydroxy-N-Methylcoclaurine;
r) a 3′-hydroxy-N-methyl-(S)-coclaurine 4′-O-methyltransferase (4′-OMT) converting (S)-3′-Hydroxy-N-Methylcoclaurine into (S)-reticuline;
s) a 1,2-dehydroreticuline synthase-1,2-dehydroreticuline reductase (DRS-DRR) converting (S)-Reticuline into ®-reticuline;
t) a salutaridine synthase (SAS) converting ®-reticuline into Salutaridine;
u) a salutaridine reductase (SAR) converting Salutaridine to Salutaridinol;
v) a salutaridinol 7-O-acetyltransferase (SAT) converting Salutaridinol into 7-O-acetylsalutaridinol;
w) a thebaine synthase (THS) converting 7-O-acetylsalutaridinol or 7-O-acetylsalutaridinol acetate into thebaine;
x) a demethylase converting thebaine into oripavine, thebaine into northebaine, oripavine into nororipavine and/or northebaine into nororipavine; and/or
y) a demethylase-CPR capable of reducing the demethylase of x).

Item 26. The host cell of the item 25, wherein the corresponding:
a) DAHP synthase has at least 70% identity to the DAHP synthase comprised in SEQ ID NO: 121;
b) chorismate mutase has at least 70% identity to the chorismate synthase comprised in SEQ ID NO: 123;
c) prephenate dehydrogenase (Tyr1) has at least 70% identity to the DAHP synthase comprised in SEQ ID NO: 125;
d) Tyrosine Hydroxylase (TH) has at least 70% identity to the TH comprised in SEQ ID NO: 127;
e) TH-CPR has at least 70% identity to the TH-CPR comprised in SEQ ID NO: 129;
f) DODC has at least 70% identity to the DODC comprised in SEQ ID NO: 131;
g) Norcoclaurine synthase (NCS) has at least 70% identity to the NCS comprised in SEQ ID NO: 133;
h) 6-OMT has at least 70% identity to the 6-OMT comprised in SEQ ID NO: 135;
i) CNMT has at least 70% identity to the CNMT comprised in SEQ ID NO: 137;
j) NMCH has at least 70% identity to the NMCH comprised in SEQ ID NO: 139;
k) 4′-OMT has at least 70% identity to the 4′-OMT comprised in SEQ ID NO: 141;
l) DRS-DRR has at least 70% identity to the VRS_DDR comprised in SEQ ID NO:143;
m) SAS has at least 70% identity to the SAS comprised in SEQ ID NO: 145;
n) SAT has at least 70% identity to the SAR comprised in SEQ ID NO: 147;
o) SAR has at least 70% identity to the SAT comprised in SEQ ID NO: 149;
p) THS has at least 70% identity to the THS comprised in SEQ ID NO: 151;
q) Demethylase has at least 70% identity to the demethylase comprised in anyone of SEQ ID NO: 153, 155, 157, 256, or 258; and
r) Demethylase-CPR has at least 70% identity to the demethylase-CPR comprised in anyone of SEQ ID NO: 159, 161, or 260.

Item 27. A cell culture comprising the recombinant microbial host cell of any preceding item plus cell growth medium.

Item 28. A method of producing one or more BIA, BIA-glycoside, oripavine or glycosylated oripavine or glucosylated oripavine, thebaine, northebaine, nororipavine, glycosylated nororipavine or glucosylated nororipavine, comprising:
(a) culturing the cell culture of item 27 at conditions allowing the cell to produce the BIA; and
(b) optionally recovering and/or isolating the BIA.

Item 29. The method of item 28, wherein step (a) comprises culturing in the pH range pH 3 to pH 6.5, such as pH 4 to 6, such as pH 4.5 or pH 5.5, for 5 minutes or longer, such as for 20 minutes or longer, such as for 30 minutes or longer, such as for 40 minutes or longer, such as for 60 minutes or longer, such as for 90 minutes, such as 1 day or longer.

Item 30. The method of item 28 or 29, further comprising contacting the nororipavine glycoside or oripavine glycoside with a glycosidase, at conditions allowing the glycosidase to catalyze separation of a glycosyl moiety from the nororipavine glycoside or oripavine glycoside to thereby obtain nororipavine or oripavine.

Item 31. The method of item 30, wherein the glycosidase is a β-glycosidase, such as β-glucosidase.

Item 32. The method of any of items 28 to 31, wherein the recovered and/or isolated BIA, BIA-glycoside, oripavine or glycosylated oripavine or glucosylated oripavine, thebaine, northebaine, nororipavine, glycosylated nororipavine or glucosylated nororipavine is converted into bis-benzyl nororipavine, nalbuphine, morphine, hydromorphone, codeine, hydrocodone, oxycodone, oxymorphone noroxymorphone, noroxymorphinone, buprenorphine, naloxone, naltrexone, or nalmefene.

Item 33. Use of the cell culture of item 27, or the one or more BIA, BIA-glycoside, oripavine or glycosylated oripavine or glucosylated oripavine, thebaine, northebaine, nororipavine or glycosylated nororipavine, glucosylated nororipavine, bis-benzyl nororipavine, nalbuphine, morphine, hydromorphone, codeine, hydrocodone, oxycodone, oxymorphone noroxymorphone, noroxymorphinone, buprenorphine, naloxone, naltrexone, or nalmefene, produced according to the method of any of items 28 to 32, in the manufacture of a medicament for the relief of pain, opioid use disorder (OUD), opioid overdose, and alcohol use disorder.

Item 34. The use of the BIA-glycoside of item 33, wherein the BIA-glycoside is gly-nororipavine or gly-oripavine.

Item 35. A pharmaceutical composition comprising the one or more BIA, BIA-glycoside, oripavine, thebaine, northebaine, nororipavine or glycosylated nororipavine, glucosylated nororipavine, bis-benzyl nororipavine, nalbuphine, morphine, hydromorphone, codeine, hydrocodone, oxycodone, oxymorphone noroxymorphone, noroxymorphinone, buprenorphine, naloxone, naltrexone, or nalmefene, produced according to the method of any of items 28 to 32, and one or more agents, additives and/or excipients.

Item 36. A pharmaceutical composition comprising one or more active pharmaceutical ingredients manufactured from one or more of the BIAs produced according to the method of any of items 28 to 32, in the manufacture of a medicament for the relief of pain, opioid use disorder (OUD), opioid overdose, and alcohol use disorder.

1. A recombinant microbial host cell capable of producing one or more BIA, BIA-glycoside, oripavine or glycosylated oripavine or glucosylated oripavine, thebaine, northebaine, nororipavine, glycosylated nororipavine or glucosylated nororipavine, wherein the host cell comprises a recombinant polynucleotide comprising a promoter operably linked to an ABC transporter effluxing one or more BIA or BIA-glycoside products.

2. The recombinant microbial host cell of claim 1, wherein one or more of the ABC transporters are one or more selected from:
a. present in the host cell at a gene copy number greater than in the wild type of the microbial host cell,
b. operably linked to a constitutive promoter or an inducible promoter that induces expression during cell exponential phase and BIA production, or
c. are under regulatory control such that a higher level of gene expression is induced by growth medium, growth conditions, the presence of an activator or transcription factor or absence of a repressor for an inducible promoter governing expression of the one or more endogenous ABC transporters, or any combination thereof.

3. The recombinant microbial host cell of claim 1 or 2, wherein the increased expression of one or more endogenous ABC transporters results from elevated levels of one or more transcription factors, wherein the one or more transcription factors are selected from:
a. polypeptide sequence having at least 75%, such as at least 80%, such as at least 85%, such as at least 90%, such as at least 95%, such as at least 99%, or 100% identity to SEQ ID No. 902, 904, 906, 908, or
b. encoded by a nucleic acid sequence having at least 75%, such as at least 80%, such as at least 85%, such as at least 90%, such as at least 95%, such as at least 99%, or 100% identity to SEQ ID No. 901, 903, 905, 907, or genomic DNA thereof.

4. The recombinant microbial host cell of any preceding claim, wherein the transcription factor is PDR 1, PDR3, PDR8 and/or YRR1.

5. A recombinant microbial host cell according to any preceding claim, wherein the recombinant microbial host cell excretes the BIA, BIA-glycoside, oripavine, glycosylated oripavine or glucosylated oripavine, thebaine, northebaine, nororipavine, glycosylated nororipavine or glucosylated nororipavine produced by the recombinant microbial host cell, at greater than 2%, preferably greater than 5%, preferably greater than 10%, preferably greater than 20% more excretion compared to a negative control recombinant microbial host cell not expressing the ABC transporter during cell exponential phase and BIA production.

6. A recombinant microbial host cell according to any preceding claim, wherein the recombinant microbial host cell produces the one or more of the BIAs at greater than 2%, preferably more than 5%, preferably more than 10%, preferably more than 20%, preferably more than 50% more than a negative control recombinant microbial host cell not expressing the ABC transporter during cell exponential phase and BIA production.

7. A recombinant microbial host cell according to any preceding claim, wherein the one or more excreted BIAs are thebaine, nororipavine, oripavine, glucosylated oripavine or glucosylated nororipavine.

8. The recombinant microbial host cell of any preceeding claim, wherein the ABC transporter is an ABC transporter involved in drug efflux or xenobiotic efflux.

9. The recombinant microbial host cell of any preceeding claim, wherein the ABC transporter comprises a Walker A sequence G(A/S/R)(S/T)GAGK(S/T), a linker sequence (L/V)SGG(E/Q), and a Walker B sequence comprising four hydrophobic residues, an optional additional fifth hydrophobic residue and a D such that (I/L)(I/L)(I/V/L)(F/L/M)XD where X represents the optional additional hydrophobic residue or no additional residue.

10. The recombinant microbial host cell of any preceding claim, wherein the ABC transporter is:
a. an ABCC/multi-drug resistance associated protein (MRP) ABC transporters, or
b. an ABCG/pleiotropic drug resistance (PDR) ABC transporters.

11. The recombinant microbial host cell of any preceding claim, wherein the ABC transporter is not native to a BIA-producing plant.

12. The recombinant microbial host cell of any preceding claim, wherein the ABCC/multi-drug resistance associated protein (MRP) ABC transporter is:
a. a polypeptide comprising a sequence having at least 45%, such as at least 60%, such as at least 70%, such as at least 80%, such as at least 85%, such as at least 90%, such as at least 95%, such as at least 99%, such as 100% identity to SEQ ID No. 872, 910, 912, 914, 918, 920, 922, 924, 926, 928, 930, 932, 934, 936, 938, 940, 942, 956, 960, 962, 964, 966, 970, 1032, 1034, 1038 or 1040 or
b. encoded by a nucleic acid sequence having at least 45%, such as at least 60%, such as at least 70%, such as at least 80%, such as at least 85%, such as at least 90%, such as at least 95%, such as at least 99%, such as 100% identity to SEQ ID No. 871, 909, 911, 913, 917, 919, 921, 923, 925, 927, 929, 931, 933, 935, 937, 939, 941, 955, 959, 961, 963, 965, 969, 1031, 1033, 1037 or 1039 or genomic DNA thereof.

13. The recombinant microbial host cell of any of claims 1 to 11, wherein the ABC transporter comprises Walker A sequences G(X)(I/V)G(S/T)GK where X is a residue selected from P, L, S, A, V or M and GRTGAGK, two linker sequences comprising LSGGQ and NFSLGE, and Walker B sequences (I/V/T)(I/Y/V)L(M/F/L)D and I(I/L)(I/V)(L/M)D.

14. The recombinant microbial host cell of any of claims 1 to 11, wherein the ABCG/pleiotropic drug resistance (PDR) ABC transporter is:
a. a polypeptide comprising a sequence having at least 45%, such as at least 60%, such as at least 75%, such as at least 80%, such as at least 85%, such as at least 90%, such as at least 95%, such as at least 99%, such as 100% identity to SEQ ID No. 916, 976, 980, 986, 988, 990, 994, 996, 1010, 1012, 1018, 1020, 1022, 1026, 1028 or 1030, or
b. encoded by a nucleic acid sequence having at least 70%, such as at least 80%, such as at least 85%, such as at least 90%, such as at least 95%, such as at least 99%, such as 100% identity to SEQ ID No. 915, 975, 979, 985, 987, 989, 993, 995, 1009, 1011, 1017, 1019, 1021, 1025, 1027 or 1029 or genomic DNA thereof.

15. The recombinant microbial host cell of any of claims 1 to 11 or claim 14, wherein the ABC transporter comprises Walker A sequences GRPGSGC(S/T) and G(A/S)SGAGKT, linker sequences VSGGERKRVSIA and LNVEQRKRLTIG, and Walker B sequences (F/L)QCWD and LL(V/L)F(L/F)D.

16. The recombinant microbial host cell of any preceding claim, further comprising:
a) one or more heterologous CYP demethylases capable of converting thebaine into northebaine, thebaine into oripavine, northebaine into nororipavine and/or oripavine into nororipavine, and one or more demethylase cytochrome P450 reductase (demethylase-CPR), and/or
b) heterologous sequences encoding:
i. a tyrosine hydroxylase (TH) converting L-tyrosine into L-dopa, and
ii. optionally, a TH-CPR capable of reducing the TH of i), and
iii. a L-dopa decarboxylase (DODC) converting L-dopa into dopamine, or a tyrosine decarboxylase (TYDC) converting L-dopa into dopamine, and
iv. a monoamine oxidase converting dopamine into 3,4-DHPAA, or a N-methyl-coclaurine hydroxylase (NMCH) converting (S)-Coclaurine into (S)-3′-hydroxycoclaurine and/or (S)—N-Methylcoclaurine into (S)-3′-Hydroxy-N-Methylcoclaurine; and
v. a norcoclaurine synthase (NCS) converting Dopamine and 4-HPAA into (S)-norcoclaurine and/or 3,4-DHPAA and dopamine to NLDS, and
vi. a 6-O-methyltransferase (6-OMT) converting (S)-norcoclaurine into (S)-Coclaurine and/or norlaudanosoline into (S)-3′-Hydroxy-coclaurine, and
vii. a coclaurine-N-methyltransferase (CNMT) converting (S)-Coclaurine into (S)—N-Methylcoclaurine and/or (S)-3′-hydroxycoclaurine into (S)-3′-hydroxy-N-methyl-coclaurine, and
viii. a 3′-hydroxy-N-methyl-(S)-coclaurine 4′-O-methyltransferase (4′-OMT) converting (S)-3′-Hydroxy-N-Methylcoclaurine into (S)-reticuline, and
ix. a 1,2-dehydroreticuline synthase-1,2-dehydroreticuline reductase (DRS-DRR) converting (S)-reticuline into (R)-reticuline comprised of one or more proteins, and
x. a salutaridine synthase (SAS) converting (R)-reticuline into Salutaridine, and
xi. a salutaridine reductase (SAR) converting Salutaridine to Salutaridinol, and
xii. a salutaridinol 7-O-acetyltransferase (SAT) converting Salutaridinol into 7-O-acetylsalutaridinol, and
xiii. a thebaine synthase (THS) converting 7-O-acetylsalutaridinol or 7-O-acetylsalutaridinol acetate into thebaine;
c) and optionally, one or more glycosyl transferases capable of transferring a glycosyl moiety to a BIA, oripavine or nororipavine.

17. The recombinant microbial host cell of claim 16, wherein the one or more demethylases is:
a. an N-demethylase comprising a polypeptide sequence having at least 75%, such as at least 85%, such as at least 90% or at least 95% identity to SEQ ID No. 140, 152, 198, 250, 252, 843, or
b. an N-demethylase encoded by a nucleic acid sequence having at least 75%, such as at least 85%, such as at least 90% or at least 95% identity to 141, 153, 199, 251, 253, 844, or genomic DNA thereof, or
c. an O-demethylase comprising a polypeptide sequence having at least 75%, such as at least 85%, such as at least 90% or at least 95% identity to SEQ ID No. 198, 222, 224, 236, or
d. an O-demethylase encoded by a nucleic acid sequence having at least 75%, such as at least 85%, such as at least 90% or at least 95% identity to SEQ ID No. 199, 223, 225, or 237, or genomic DNA thereof.

18. The recombinant microbial host cell of claim 16 or 17, wherein the one or more CPRs:
a. comprises a polypeptide sequence having 75%, such as at least 85%, such as at least 90% or at least 95% identity to SEQ ID No. 292 or 305, or
b. is encoded by a nucleic acid sequence having at least 75%, such as at least 85%, such as at least 90% or at least 95% identity to SEQ ID No. 293 or 306, or genomic DNA thereof.

19. The recombinant microbial host cell of any preceding claim, wherein the one or more glycosyltransferases (UGT) is an aglycone O-UGT or an aglycone O-glucosyltransferase.

20. The recombinant microbial host cell of any preceding claim, wherein the one or more glycosyltransferases (UGT):
a. comprises an amino acid sequence having at least 60%, such as at least 70%, such at least 75%, such as at least 80%, such as at least 90%, such as at least 95%, such as at least 99%, such as 100% identity to a UGT comprised in any one of SEQ ID NO: 880, 882, 878, 884, 886, 888, 890, 892, 894, 896, or 898; or
b. is encoded by a nucleic acid sequence having at least 60%, such as at least 70%, such at least 75%, such as at least 80%, such as at least 90%, such as at least 95%, such as at least 99%, such as 100% identity to SEQ ID No. 879, 881, 877, 883, 885, 887, 889, 891, 893, 895 or 897, or genomic DNA thereof.

21. The recombinant microbial host cell of any preceding claim, wherein the recombinant microbial host cell is a yeast.

22. The recombinant microbial host cell of any preceding claim, wherein the recombinant microbial host cell is Saccharomyces cerevisiae.

23. The recombinant microbial host cell of any preceding claim, further comprising an uptake transporter capable of transporting an opioid, such as oripavine, into the recombinant host cell.

24. The recombinant microbial host cell of claim 23, wherein the uptake transporter is a polypeptide:
a. comprising an amino acid sequence having at least 60%, such as at least 70%, such at least 75%, such as at least 80%, such as at least 90%, such as at least 95%, such as at least 99%, such as 100% identity to an uptake transporter comprised in any one of SEQ ID NO: 307, 311, 317, 461, 473, 733, or 735.
b. encoded by a nucleic acid sequence comprising at least 60%, such as at least 70%, such at least 75%, such as at least 80%, such as at least 90%, such as at least 95%, such as at least 99%, such as 100% identity to 308, 312, 318, 462, 474, 734, 736, or genomic DNA thereof.

25. The recombinant microbial host cell of any preceding claim, further comprising an operative biosynthetic pathway capable of producing the thebaine, northebaine, oripavine and/or nororipavine, wherein the pathway comprises one or more polypeptides selected from:
a) a 3-deoxy-D-arabino-2-heptulosonic acid 7-phosphate synthase (DAHP synthase) converting PEP and E4P into DAHP;
b) a 5-enolpyruvylshikimate-3-phosphate (EPSP) synthase (aro1) converting 3-phosphoshikimate and PEP into EPSP;
c) an aro1 polypeptide converting DHAP and PEP into EPSP;
d) a chorismate synthase converting EPSP into Chorismate;
e) a chorismate mutase converting Chorismate into prephenate;
f) a prephenate dehydrogenase (Tyr1) converting prephenate into 4-HPP;
g) an aromatic aminotransferase converting 4-HPP into L-Tyrosine;
h) a tyrosine hydroxylase (TH) converting L-tyrosine into L-dopa
i) a TH-CPR capable of reducing the TH of h);
j) a L-dopa decarboxylase (DODC) converting L-dopa into dopamine;
k) a Tyrosine decarboxylase (TYDC) converting L-dopa into dopamine;
l) a hydroxyphenylpyruvate decarboxylase (HPPDC) converting 4-HPP into 4-HPPA;
m) a monoamine oxidase converting dopamine into 3,4-DHPAA;
n) a norcoclaurine synthase (NCS) converting Dopamine and 4-HPAA into (S)-norcoclaurine;
o) a 6-O-methyltransferase (6-OMT) converting (S)-norcoclaurine into (S)-Coclaurine and/or norlaudanosoline into (S)-3′-Hydroxy-coclaurine;
p) a coclaurine-N-methyltransferase (CNMT) converting (S)-Coclaurine into (S)—N-Methylcoclaurine and/or (S)-3′-hydroxycoclaurine into (S)-3′-hydroxy-N-methyl-coclaurine;
q) a N-methyl-coclaurine hydroxylase (NMCH) converting (S)-Coclaurine into (S)-3′-hydroxycoclaurine and/or (S)—N-Methylcoclaurine into (S)-3′-Hydroxy-N-Methylcoclaurine;
r) a 3′-hydroxy-N-methyl-(S)-coclaurine 4′-O-methyltransferase (4′-OMT) converting (S)-3′-Hydroxy-N-Methylcoclaurine into (S)-reticuline;
s) a 1,2-dehydroreticuline synthase-1,2-dehydroreticuline reductase (DRS-DRR) converting (S)-Reticuline into (R)-reticuline;
t) a salutaridine synthase (SAS) converting (R)-reticuline into Salutaridine;
u) a salutaridine reductase (SAR) converting Salutaridine to Salutaridinol;
v) a salutaridinol 7-O-acetyltransferase (SAT) converting Salutaridinol into 7-O-acetylsalutaridinol;
w) a thebaine synthase (THS) converting 7-O-acetylsalutaridinol or 7-O-acetylsalutaridinol acetate into thebaine;
x) a demethylase converting thebaine into oripavine, thebaine into northebaine, oripavine into nororipavine and/or northebaine into nororipavine; and/or
y) a demethylase-CPR capable of reducing the demethylase of x).

26. The host cell of the claim 25, wherein the corresponding:
a) DAHP synthase has at least 70% identity to the DAHP synthase comprised in SEQ ID NO: 121;
b) chorismate mutase has at least 70% identity to the chorismate synthase comprised in SEQ ID NO: 123;
c) prephenate dehydrogenase (Tyr1) has at least 70% identity to the DAHP synthase comprised in SEQ ID NO: 125;
d) Tyrosine Hydroxylase (TH) has at least 70% identity to the TH comprised in SEQ ID NO: 127;
e) TH-CPR has at least 70% identity to the TH-CPR comprised in SEQ ID NO: 129;
f) DODC has at least 70% identity to the DODC comprised in SEQ ID NO: 131;
g) Norcoclaurine synthase (NCS) has at least 70% identity to the NCS comprised in SEQ ID NO: 133;
h) 6-OMT has at least 70% identity to the 6-OMT comprised in SEQ ID NO: 135;
i) CNMT has at least 70% identity to the CNMT comprised in SEQ ID NO: 137;
j) NMCH has at least 70% identity to the NMCH comprised in SEQ ID NO: 139;
k) 4′-OMT has at least 70% identity to the 4′-OMT comprised in SEQ ID NO: 141;
l) DRS-DRR has at least 70% identity to the VRS_DDR comprised in SEQ ID NO:143;
m) SAS has at least 70% identity to the SAS comprised in SEQ ID NO: 145;
n) SAT has at least 70% identity to the SAR comprised in SEQ ID NO: 147;
o. SAR has at least 70% identity to the SAT comprised in SEQ ID NO: 149;
p) THS has at least 70% identity to the THS comprised in SEQ ID NO: 151;
q) Demethylase has at least 70% identity to the demethylase comprised in anyone of SEQ ID NO: 153, 155, 157, 256, or 258; and
r) Demethylase-CPR has at least 70% identity to the demethylase-CPR comprised in anyone of SEQ ID NO: 159, 161, or 260.

27. A cell culture comprising the recombinant microbial host cell of any preceding claim plus cell growth medium.

28. A method of producing one or more BIA, BIA-glycoside, oripavine or glycosylated oripavine or glucosylated oripavine, thebaine, northebaine, nororipavine, glycosylated nororipavine or glucosylated nororipavine, comprising:
(a) culturing the cell culture of claim 27 at conditions allowing the cell to produce the BIA; and
(b) optionally recovering and/or isolating the BIA.

29. The method of claim 28, wherein step (a) comprises culturing in the pH range pH 3 to pH 6.5, such as pH 4 to 6, such as pH 4.5 or pH 5.5, for 5 minutes or longer, such as for 20 minutes or longer, such as for 30 minutes or longer, such as for 40 minutes or longer, such as for 60 minutes or longer, such as for 90 minutes, such as 1 day or longer.

30. The method of claim 28 or 29, further comprising contacting the nororipavine glycoside or oripavine glycoside with a glycosidase, at conditions allowing the glycosidase to catalyze separation of a glycosyl moiety from the nororipavine glycoside or oripavine glycoside to thereby obtain nororipavine or oripavine.

31. The method of claim 30, wherein the glycosidase is a β-glycosidase, such as β-glucosidase.

32. The method of any of claims 28 to 31, wherein the recovered and/or isolated BIA, BIA-glycoside, oripavine or glycosylated oripavine or glucosylated oripavine, thebaine, northebaine, nororipavine, glycosylated nororipavine or glucosylated nororipavine is converted into bis-benzyl nororipavine, nalbuphine, morphine, hydromorphone, codeine, hydrocodone, oxycodone, oxymorphone noroxymorphone, noroxymorphinone, buprenorphine, naloxone, naltrexone, or nalmefene.

33. Use of the cell culture of claim 27, or the one or more BIA, BIA-glycoside, oripavine or glycosylated oripavine or glucosylated oripavine, thebaine, northebaine, nororipavine or glycosylated nororipavine, glucosylated nororipavine, bis-benzyl nororipavine, nalbuphine, morphine, hydromorphone, codeine, hydrocodone, oxycodone, oxymorphone noroxymorphone, noroxymorphinone, buprenorphine, naloxone, naltrexone, or nalmefene, produced according to the method of any of claims 28 to 32, in the manufacture of a medicament for the relief of pain, opioid use disorder (OUD), opioid overdose, and alcohol use disorder.

34. The use of the BIA-glycoside of claim 33, wherein the BIA-glycoside is gly-nororipavine or gly-oripavine.

35. A pharmaceutical composition comprising the one or more BIA, BIA-glycoside, oripavine, thebaine, northebaine, nororipavine or glycosylated nororipavine, glucosylated nororipavine, bis-benzyl nororipavine, nalbuphine, morphine, hydromorphone, codeine, hydrocodone, oxycodone, oxymorphone noroxymorphone, noroxymorphinone, buprenorphine, naloxone, naltrexone, or nalmefene, produced according to the method of any of claims 28 to 32, and one or more agents, additives and/or excipients.

36. A pharmaceutical composition comprising one or more active pharmaceutical ingredients manufactured from one or more of the BIAs produced according to the method of any of claims 28 to 32, in the manufacture of a medicament for the relief of pain, opioid use disorder (OUD), opioid overdose, and alcohol use disorder.
